(19) United States
(12) Patent Application Publication
(10) Pub. No.: US 2026/0217697 A1
(43) Pub. Date: Jul. 30, 2026

(54) COMPOUNDS COMPRISING HETEROARYL RINGS AND COMPOSITIONS AND METHODS THEREOF

(71) Applicant: lonGen Therapeutics Co., Ltd., Nanjing (CN)

(72) Inventors: Xiaoguang Lei, Beijing (CN); Daohong Liao, Ningbo (CN); Yong Yang, Nanjing (CN); Linghan Hu, Nanjing (CN); Shun Su, San Diego, CA (US)

(21) Appl. No.: 19/143,360

(22) PCT Filed: Dec. 22, 2023
(86) PCT No.: PCT/CN2023/140832

§ 371 Date: Jun. 25, 2025

(30) Foreign Application Priority Data
Dec. 26, 2022 (WO) PCT/CN2022/141788

(51) Int. Cl. C07D 417/12; A61K 31/426; A61K 31/437; A61K 33/04; C07D 513/04; C07D 513/14; C07D 513/20; C07D 517/20

(52) U.S. Cl. CPC C07D 417/12; A61K 31/426; A61K 31/437; A61K 33/04; C07D 513/04; C07D 513/14; C07D 513/20; C07D 517/20

(57) ABSTRACT

Among other things, the present disclosure provides compounds of formula I or salts thereof. In some embodiments, provided compounds are useful as TRPV3 inhibitors. In some embodiments, the present disclosure provides methods for preventing or treating TRPV3-associated conditions, disorders or diseases.

### CROSS-REFERENCE TO RELATED APPLICATIONS

This application claims priority to PCT Application No. PCT/CN2022/141788, filed on Dec. 26, 2022, the entirey of which is incorporated herein by reference.

### TECHNICAL FIELD

In some embodiments, the present disclosure provides compounds and compositions, among other things, useful for modulating Transient Receptor Potential Vanilloid 3 (TRPV3) activities. In some embodiments, provided compounds and compositions are useful for preventing or treating TRPV3 associated conditions, disorders or diseases, e.g., pain, itch, skin inflammation, hair loss, etc. In some embodiments, the present disclosure provides technologies for preparing provided compounds and compositions.

### BACKGROUND

Compounds are useful for many purposes including modulating biological functions and activities. It has been reported that transient receptor potential (TRP) channels have a number of biological functions and can be associated with various conditions, disorders or diseases, e.g., pain, itch, and hair loss. TRPV3 has been reported to be a member of the vanilloid TRP (TRPV) subfamily.

### SUMMARY

In some embodiments, the present disclosure provides various compounds, e.g., compounds having the structure of formula I or salts thereof, and compositions and methods thereof. In some embodiments, the present disclosure provides a compound, wherein the compound has the structure of formula I:

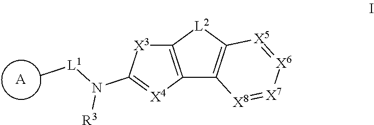

or a salt thereof, wherein:
Ring A is an optionally substituted 5-6 membered aromatic ring having 0-4 heteroatoms, or is

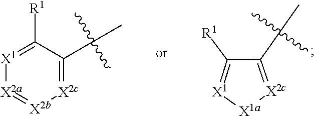

each of X¹, X^{2a}, X^{2b}, and X^{2c} is independently —N═, C(R²)═ or optionally substituted —CH═;
X^{1a} is —O—, —S—, —N(R′), C(R′)₂—, or optionally substituted —CH₂— or —NH—;
X³ is —O—, —S—, —Se—, —N(R′)— or optionally substituted —NH—;
each of X⁴, X⁵, X⁶, X⁷ and X⁸ is independently —N═, C(R⁶)═ or optionally substituted —CH═;
each of R¹, R², R^{s} and R⁶ is independently halogen, CN, —NO₂, -L-R′, —OR′, —N(R′)₂, —N(R′)C(O)OR′, —C(O)R′, —C(O)OR′, —C(O)N(R′)₂, or —OC(O)N(R′)₂;
L′ is a covalent bond, —C(O)—, —C(S)—, —S(O)₂—, —N(R′)C(O)—, —N(R′)S(O)₂—, —N(R′)C(S)— or

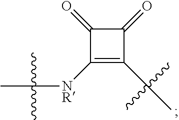

each of R³, R⁴ and R⁵ is independently R′;
each of L and L² is independently optionally substituted C₁₋₃ alkylene, wherein one or more methylene unit of the alkylene is optionally and independently replaced with —O—, —S—, —C(R⁴)(R⁵)—, —N(R⁴)—, optionally substituted —CH═N—, optionally substituted —CH═CH— or -Cy-;
each -Cy- is independently

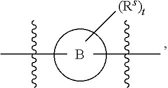

wherein Ring B is an optionally substituted saturated or partially saturated 3-10 membered ring having 0-4 heteroatoms;
t is 0, 1, 2, 3 or 4;
each R′ is independently R, —OR, —C(O)R, —C(O)OR, or —S(O)₂R;
each R is independently hydrogen or an optionally substituted group selected from C₁-C₁₀ aliphatic, C₁-C₁₀ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₁₀ aliphatic, and 5-10 membered heteroaryl having 1-6 heteroatoms-C₁-C₁₀ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

In some embodiments, the present disclosure provides a pharmaceutical composition of a provided compound. In some embodiments, the present disclosure provides a pharmaceutical composition comprising a provided compound or a pharmaceutically acceptable salt thereof, and a pharmaceutically acceptable carrier. In some embodiments, the present disclosure provides a pharmaceutical composition delivering a provided compound or a pharmaceutically acceptable salt thereof, and a pharmaceutically acceptable carrier.

Provided technologies are useful for many purposes. In some embodiments, provided compounds are useful as TRPV3 modulators. In some embodiments, provided technologies (e.g., compounds, compositions, methods, etc.) are useful for preventing or treating various conditions, disorders or diseases. In some embodiments, a condition, disorder or disease is a TRPV3-associated condition, disorder or disease. In some embodiments, a condition, disorder or disease is or comprises itch. In some embodiments, a condition, disorder or disease is or comprises pain. In some embodiments, a condition, disorder or disease is or comprises skin inflammation. In some embodiments, a condition, disorder or disease is or comprises hair loss. In some embodiments, a condition, disorder or disease is associated with administration of another therapeutic agent.

In some embodiments, the present disclosure provides a method for preventing a condition, disorder or disease, comprising administering or delivering to a subject susceptible thereto an effective amount of a provided compound, e.g., a compound of formula I or a salt thereof. In some embodiments, the present disclosure provides a method for treating a condition, disorder or disease, comprising administering or delivering to a subject suffering therefrom an effective amount a provided compound, e.g., a compound of formula I or a salt thereof. In some embodiments, a condition, disorder or disease is a TRPV3-associated condition, disorder or disease. In some embodiments, a condition, disorder or disease is associated with TRPV3 activation. In some embodiments, a condition, disorder or disease is or comprises itch. In some embodiments, a condition, disorder or disease is or comprises pain. In some embodiments, a condition, disorder or disease is or comprises hair loss.

In some embodiments, the present disclosure provides a method for preventing a condition, disorder or disease, comprising administering or delivering to a subject susceptible thereto an effective amount a provided compound and another agent. In some embodiments, the present disclosure provides a method for treating a condition, disorder or disease, comprising administering or delivering to a subject suffering therefrom an effective amount a provided compound and another agent.

In some embodiments, the present disclosure provides technologies, e.g., methods, reagents, etc., for preparing a compound of formula I or a pharmaceutical acceptable salt thereof.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise clear from context, isomers of compounds are included. As appreciated by those skilled in the art, compounds may be provided, administered, or delivered in various forms, e.g., salts (e.g., pharmaceutically acceptable salts), solvates, hydrates, esters, prodrugs, tautomers, etc.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1~4 carbon atoms (e.g., C₁-C₄ for straight chain lower alkyls).

Alkynyl: As used herein, the term “alkynyl” refers to an aliphatic group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non aromatic rings, such as indanyl, phthalimidyl, naphthimidyl, or tetrahydronaphthyl, and the like.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 x electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents include halogen; —(CH₂)₀₋₄R^{○}; —(CH₂)₀₋₄OR^{○}; O(CH₂)₀₋₄R^{○}, —O—(CH₂)₀₋₄C(O)OR^{○}; —(CH₂)₀₋₄CH(OR^{○})₂; —(CH₂)₀₋₄Ph, which may be substituted with R^{○}; —(CH₂)₀₋₄O(CH₂)₀₋₁Ph which may be substituted with R^{○}; —CH═CHPh, which may be substituted with R^{○}; —(CH₂)₀₋₄O(CH₂)₀₋₁-pyridyl which may be substituted with R^{○}; —NO₂; —CN; —N₃; —(CH₂)₀₋₄N(R^{○})₂; —(CH₂)₀₋₄N(R^{○})C(O)R^{○}; —N(R^{○})C(S)R^{○}; —(CH₂)₀₋₄N(R^{○})C(O)NR^{○}₂; —N(R^{○})C(S)NR^{○}₂; —(CH₂)₀₋₄N(R^{○})C(O)OR^{○}; —N(R^{○})N(R^{○})C(O)R^{○}; —N(R^{○})N(R^{○})C(O)NR^{○}₂; —N(R^{○})N(R^{○})C(O)OR^{○}; —(CH₂)₀₋₄C(O)R^{○}; —C(S)R^{○}; —(CH₂)₀₋₄C(O)OR^{○}; —(CH₂)₀₋₄C(O)SR^{○}; —(CH₂)₀₋₄C(O)OSiR^{○}₃; —(CH₂)₀₋₄OC(O)R^{○}; —OC(O) (CH₂)₀₋₄SR, SC(S)SR^{○}; —(CH₂)₀₋₄SC(O)R^{○}; —(CH₂)₀₋₄C(O)NR^{○}₂; C(S)NR^{○}₂; C(S)SR^{○}; —SC(S)SR^{○}, —(CH₂)₀₋₄OC(O)NR^{○}₂; —C(O)N(OR^{○})R^{○}; —C(O)C(O)R^{○}; —C(O)CH₂C(O)R^{○}; —C(NOR^{○})R^{○}; —(CH₂)₀₋₄SSR^{○}; —(CH₂)₀₋₄S(O)₂R^{○}; —(CH₂)₀₋₄S(O)₂OR^{○}; —(CH₂)₀₋₄OS(O)₂R^{○}; —S(O)₂NR^{○}₂; —(CH₂)₀₋₄S(O)R^{○}; —N(R^{○})S(O)₂NR^{○}₂; —N(R^{○})S(O)₂R^{○}; —N(OR^{○})R^{○}; —C(NH)NR^{○}₂; —P(O)₂R^{○}; —P(O)R^{○}₂; —OP(O)R^{○}₂; —OP(O)(OR^{○})₂; —SiR^{○}₃; —OSiR^{○}₃; —(C₁₋₄ straight or branched alkylene)O—N(R^{○})₂; or —(C₁₋₄ straight or branched) alkylene)C(O)O—N(R^{○})₂, wherein each R^{○} may be substituted as defined below and is independently hydrogen, C₁₋₁₀ (e.g., C₁₋₉, C₁₋₆, C₁₋₅, C₁₋₄, etc.) aliphatic, C₁₋₁₀ (e.g., C₁₋₉, C₁₋₆, C₁₋₅, C₁₋₄, etc.) heteroaliphatic having 1-5 heteroatoms independently selected from nitrogen, oxygen, sulfur, silicon and phosphorus, —CH₂—(C₆₋₁₄ (e.g., C₆₋₁₀, C₆, etc.) aryl), —O(CH₂)₀₋₁ (C₆₋₁₄ (e.g., C₆₋₁₀, C₆, etc.) aryl), —CH₂-(5-14 (e.g., 5-10, 5-6, 5, 6, 9, 10, 14, etc.) membered heteroaryl ring having 1-5 heteroatoms independently selected from nitrogen, oxygen, and sulfur), a 3-10 (e.g., 3-9, 3-7, 3-6, 5-10, 5-6, etc.) membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{○}, taken together with their intervening atom(s), form a 3-10 (e.g., 3-9, 3-7, 3-6, 5-10, 5-6, etc.) membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{○} (or the ring formed by taking two independent occurrences of R^{○} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur. Suitable divalent substituents on a saturated carbon atom of R^{○} include ═O and ═S.

Suitable divalent substituents include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3-6 (e.g., 4-6, 5-6, etc.) membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 3-6 (e.g., 4-6, 5-6, etc.) membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur.

In some embodiments, suitable substituents on a substitutable nitrogen include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6 (e.g., 4-6, 5-6, etc.) membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12 (e.g., 3-10, 3-7, 3-6, 5-10, 5-7, 5-6, etc.) membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises two or more acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl) methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2 dimethoxycarbonylvinyl carbamate, o-(N,N-dimethylcarboxamido) benzyl carbamate, 1,1-dimethyl-3-(N,N-dimethylcarboxamido)propyl carbamate, 1,1-dimethylpropynyl carbamate, di(2-pyridyl) methyl carbamate, 2-furanylmethyl carbamate, 2-iodoethyl carbamate, isoborynl carbamate, isobutyl carbamate, isonicotinyl carbamate, p-(p′ methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo) benzyl carbamate, 2,4,6-tri-t-butylphenyl carbamate, 4-(trimethylammonium) benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, 0-nitrophenoxyacetamide, acetoacetamide, (N′-dithiobenzyloxycarbonylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4 chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro 4 pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyrolin-3-yl)amine, quaternary ammonium salts, N-benzylamine, N-di(4 methoxyphenyl)methylamine, N-5-dibenzosuberylamine, N-triphenylmethylamine (Tr), N-[(4 methoxyphenyl)diphenylmethyl]amine (MMTr), N-9-phenylfluorenylamine (PhF), N-2,7-dichloro-9-fluorenylmethyleneamine, N-ferrocenylmethylamine (Fcm), N-2-picolylamine N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten) carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4 methoxybenzenesulfenamide, triphenylmethylsulfenamide, 3-nitropyridinesulfenamide (Npys), p toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6 dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6 sulfonamide (Pmc), methanesulfonamide (Ms), B trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, o-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy) methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2 (trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4 methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl) bis(4′,4″-dimethoxyphenyl)methyl, 1,1-bis(4-methoxyphenyl)-1′-pyrenylmethyl, 9-anthryl, 9-(9 phenyl) xanthenyl, 9 (9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4 (ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2 (phenylsulfonyl)ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4 dimethoxybenzyl carbonate, alkyl o-nitrobenzyl carbonate, alkyl p-nitrobenzyl carbonate, alkyl S benzyl thiocarbonate, 4 ethoxy-1-napththyl carbonate, methyl dithiocarbonate, 2-iodobenzoate, 4 azidobutyrate, 4 nitro 4 methylpentanoate, o-(dibromomethyl)benzoate, 2-formylbenzenesulfonate, 2 (methylthiomethoxy)ethyl, 4 (methylthiomethoxy) butyrate, 2-(methylthiomethoxymethyl)benzoate, 2,6-dichloro-4-methylphenoxyacetate, 2,6 dichloro 4 (1,1,3,3-tetramethylbutyl)phenoxyacetate, 2,4 bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene acetal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, a (N,N′-dimethylamino)benzylidene derivative, 2-oxacyclopentylidene ortho ester, di-t-butylsilylene group (DTBS), 1,3 (1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl(trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl (DMTr) and 4,4′,4″-trimethoxytrityl (TMTr), 2-cyanoethyl (CE or Cne), 2-(trimethylsilyl) ethyl (TSE), 2-(2-nitrophenyl)ethyl, 2-(4-cyanophenyl) ethyl 2-(4-nitrophenyl) ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy) trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl) xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl) ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl) aminoethyl, or 4-[N-methyl-N-(2,2,2-trifluoroacetyl)amino]butyl.

Subject: As used herein, the term “subject” or “test subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

As those skilled in the art will appreciate, methods and compositions described herein relating to provided compounds generally also apply to pharmaceutically acceptable salts of such compounds.

### CERTAIN EMBODIMENTS OF COMPOUNDS

Among other things, the present disclosure provides compounds that are useful for various purposes. In some embodiments, a provided compound has the structure of formula I:

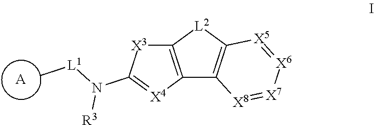

or a salt thereof, wherein:
Ring A is an optionally substituted 5-6 membered aromatic ring having 0-4 heteroatoms, or is

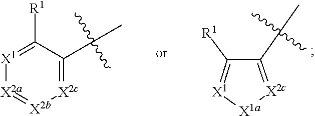

each of X¹, X^{2a}, X^{2b}, and X^{2c} is independently —N═, —C(R²)═ or optionally substituted —CH═;
X^{1a} is —O—, —S—, —N(R′)—, —C(R′)₂—, or optionally substituted —CH₂— or —NH—;
X³ is —O—, —S—, —Se—, —N(R′)— or optionally substituted —NH—;
each of X⁴, X⁵, X⁶, X⁷ and X⁸ is independently —N═, C(R⁶)═ or optionally substituted —CH═;
each of R¹, R², R^{s} and R⁶ is independently halogen, —CN, —NO₂, -L-R′, —OR′, —N(R′)₂, —N(R′)C(O)OR′, —C(O)R′, —C(O)OR′, —C(O)N(R′)₂, or —OC(O)N(R′)₂;
L¹ is a covalent bond, —C(O)—, —C(S)—, —S(O)₂—, —N(R′)C(O)—, —N(R′)S(O)₂—, —N(R′)C(S)— or

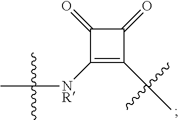

each of R³, R⁴ and R⁵ is independently R′;
each of L and L² is independently optionally substituted C₁₋₃ alkylene, wherein one or more methylene unit of the alkylene is optionally and independently replaced with —O—, —S—, —C(R⁴)(R⁵)—, —N(R⁴)—, optionally substituted —CH═N—, optionally substituted —CH═CH— or -Cy-;
each -Cy- is independently

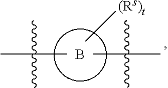

wherein Ring B is an optionally substituted saturated or partially saturated 3-10 membered ring having 0-4 heteroatoms;
t is 0, 1, 2, 3 or 4;
each R′ is independently R, OR, —C(O)R, C(O)OR, or —S(O)₂R;
each R is independently hydrogen or an optionally substituted group selected from C₁-C₁₀ aliphatic, C₁-C₁₀ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₁₀ aliphatic, and 5-10 membered heteroaryl having 1-6 heteroatoms-C₁-C₁₀ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

Certain embodiments for various variables in various formulae (e.g., formula I) are described herein as examples. Those skilled in the art reading the present disclosure will be able to select an embodiment for each variable and combine them; such combinations are within the scope the present disclosure. Those skilled in the art also appreciate that embodiments described for one variable (e.g., R) may be utilized for other variables that can be such variable (e.g., R¹, R′, R², R³, R⁴, R⁵, R^{s}, etc. that can be R).

### Ring A

In some embodiments, Ring A is an optionally substituted phenyl ring. In some embodiments, Ring A is phenyl.

In some embodiments, Ring A is an optionally substituted 5-6 membered heteroaryl ring having 1-4 heteroatoms. In some embodiments, Ring A is an optionally substituted 6-membered heteroaryl ring having 1-4 heteroatoms. In some embodiments, Ring A is an optionally substituted 6-membered heteroaryl ring having 1-3 heteroatoms. In some embodiments, Ring A is an optionally substituted 6-membered heteroaryl ring having 1-2 heteroatoms. In some embodiments, Ring A is an optionally substituted 6-membered heteroaryl ring having 1-2 heteroatoms independently selected from O, N, and S. In some embodiments, Ring A is an optionally substituted 6-membered heteroaryl ring having 2 heteroatoms. In some embodiments, Ring A is an optionally substituted 6-membered heteroaryl ring having a heteroatom selected from O, N, and S. In some embodiments, Ring A is an optionally substituted 6-membered heteroaryl ring having a nitrogen atom. In some embodiments, Ring A is an optionally substituted 6-membered heteroaryl ring having 2 heteroatoms independently selected from O, N, and S. In some embodiments, Ring A is an optionally substituted 6-membered heteroaryl ring having 2 heteroatoms each of which is N. In some embodiments, Ring A is an optionally substituted 6-membered heteroaryl ring having 3 heteroatoms. In some embodiments, Ring A is an optionally substituted 6-membered heteroaryl ring having 3 heteroatoms independently selected from O, N, and S. In some embodiments, Ring A is an optionally substituted 6-membered heteroaryl ring having 3 heteroatoms wherein one is N.

In some embodiments, Ring A is an optionally substituted 5-membered heteroaryl ring having 1-4 heteroatoms. In some embodiments, Ring A is an optionally substituted 5-membered heteroaryl ring having 1-3 heteroatoms. In some embodiments, Ring A is an optionally substituted 5-membered heteroaryl ring having 1-2 heteroatoms. In some embodiments, Ring A is an optionally substituted 5-membered heteroaryl ring having 1-2 heteroatoms independently selected from O, N, and S. In some embodiments, Ring A is an optionally substituted 5-membered heteroaryl ring having 2 heteroatoms. In some embodiments, Ring A is an optionally substituted 5-membered heteroaryl ring having 1 heteroatom. In some embodiments, Ring A is an optionally substituted 5-membered heteroaryl ring having an oxygen atom. In some embodiments, Ring A is an optionally substituted 5-membered heteroaryl ring having a nitrogen atom. In some embodiments, Ring A is an optionally substituted 5-membered heteroaryl ring having a sulfur atom. In some embodiments, Ring A is an optionally substituted 5-membered heteroaryl ring having 2 heteroatoms independently selected from O, N, and S. In some embodiments, Ring A is an optionally substituted 5-membered heteroaryl ring having 2 heteroatoms one of which is N. In some embodiments, Ring A is an optionally substituted 5-membered heteroaryl ring having 2 heteroatoms independently selected from N and S. In some embodiments, Ring A is an optionally substituted 5-membered heteroaryl ring having 2 heteroatoms independently selected from N and O. In some embodiments, Ring A is an optionally substituted 5-membered heteroaryl ring having 2 heteroatoms independently selected from O and S. In some embodiments, Ring A is an optionally substituted 5-membered heteroaryl ring having 3 heteroatoms. In some embodiments, Ring A is an optionally substituted 5-membered heteroaryl ring having 3 heteroatoms independently selected from O, N, and S. In some embodiments, a ring has a single heteroatom. In some embodiments, a ring has two or more heteroatoms at least one of which is nitrogen. In some embodiments, each is nitrogen. In some embodiments, all heteroatoms are the same. In some embodiments, at least one heteroatom is different from the other heteroatom(s).

In some embodiments, Ring A is

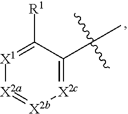

wherein each variable is independently as described herein. In some embodiments, Ring A is

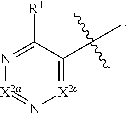

wherein each variable is independently as described herein. In some embodiments, Ring A is

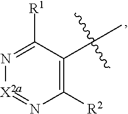

wherein each variable is independently as described herein. In some embodiments, Ring A is

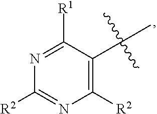

wherein each variable is independently as described herein. In some embodiments, Ring A is

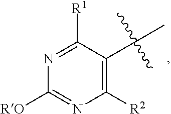

wherein each variable is independently as described herein. In some embodiments, Ring A is

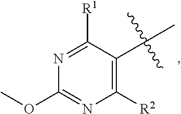

wherein each variable is independently as described herein. In some embodiments, Ring A is

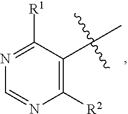

wherein each variable is independently as described herein. In some embodiments, R¹ is —OR′ wherein R′ is as described herein. In some embodiments, R² is —OR′ wherein R′ is as described herein. In some embodiments, each of R¹ and R² is independently —OR′ wherein R′ is as described herein. In some embodiments, R¹ and R² are the same. In some embodiments, R¹ and R² are different. In some embodiments, R′ is optionally substituted C₁₋₆ aliphatic. In some embodiments, R′ is optionally substituted C₁₋₆ alkyl. In some embodiments, R′ is optionally substituted methyl. In some embodiments, R′ is methyl. In some embodiments, Ring A is

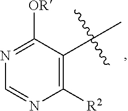

wherein each variable is independently as described herein. In some embodiments, Ring A is

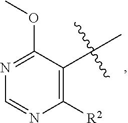

wherein each variable is independently as described herein. In some embodiments, Ring A is

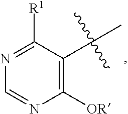

wherein each variable is independently as described herein. In some embodiments, Ring A is

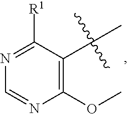

wherein each variable is independently as described herein. In some embodiments, Ring A is

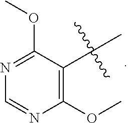 [SMILES: *c1c(OC)ncnc1OC]

In some embodiments, Ring A is optionally substituted pyrimidinyl. In some embodiments, Ring A is optionally substituted 2′-pyrimidinyl. In some embodiments, Ring A is optionally substituted 4′-pyrimidinyl. In some embodiments, Ring A is optionally substituted

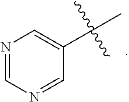 [SMILES: *c1cncnc1]

In some embodiments, Ring A is

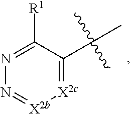

wherein each variable is independently as described herein. In some embodiments, Ring A is

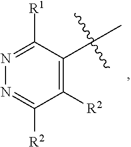

wherein each variable is independently as described herein. In some embodiments, Ring A is

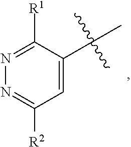

wherein each variable is independently as described herein. In some embodiments, Ring A is

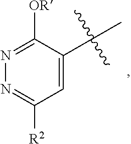

wherein each variable is independently as described herein. In some embodiments, Ring A is

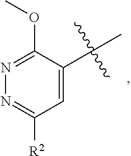

wherein each variable is independently as described herein. In some embodiments, Ring A is

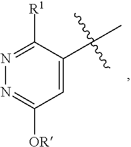

wherein each variable is independently as described herein. In some embodiments, Ring A is

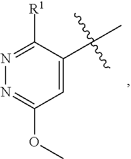

wherein each variable is independently as described herein. In some embodiments, Ring A is

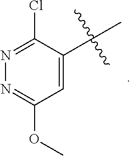 [SMILES: *c1cc(OC)nnc1Cl]

In some embodiments, Ring A is

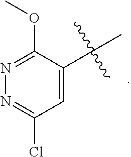 [SMILES: *c1cc(Cl)nnc1OC]

In some embodiments, Ring A is optionally substituted pyridazinyl. In some embodiments, Ring A is optionally substituted 3′-pyridazinyl. In some embodiments, Ring A is optionally substituted

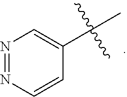 [SMILES: *c1ccnnc1]

In some embodiments, Ring A is

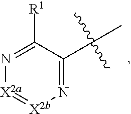

wherein each variable is independently as described herein. In some embodiments, Ring A is

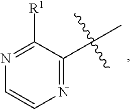

wherein each variable is independently as described herein. In some embodiments, Ring A is

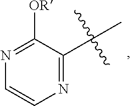

wherein each variable is independently as described herein. In some embodiments, Ring A is

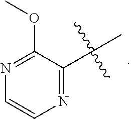 [SMILES: *c1nccnc1OC]

In some embodiments, Ring A is optionally substituted

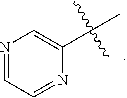 [SMILES: *c1cnccn1]

In some embodiments, Ring A is

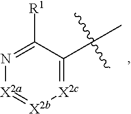

wherein each variable is independently as described herein. In some embodiments, Ring A is

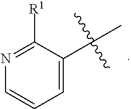

wherein each variable is independently as described herein. In some embodiments, Ring A is

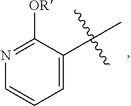

wherein each variable is independently as described herein. In some embodiments, Ring A is

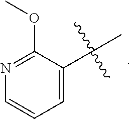 [SMILES: *c1cccnc1OC]

In some embodiments, Ring A is optionally substituted pyridinyl. In some embodiments, Ring A is optionally substituted 2′-pyridinyl. In some embodiments, Ring A is optionally substituted 3′-pyridinyl. In some embodiments, Ring A is optionally substituted 4′-pyridinyl.

In some embodiments, Ring A is

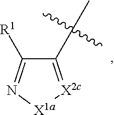

wherein each variable is independently as described herein. In some embodiments, Ring A is

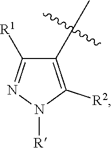

wherein each variable is independently as described herein. In some embodiments, Ring A is

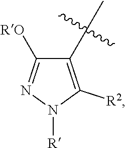

wherein each variable is independently as described herein. In some embodiments, Ring A is

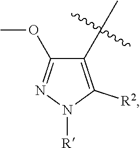

wherein each variable is independently as described herein. In some embodiments, Ring A is

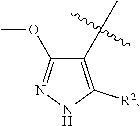

wherein each variable is independently as described herein. In some embodiments, Ring A is

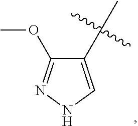 [SMILES: *c1c[nH]nc1OC]

wherein each variable is independently as described herein. In some embodiments, Ring A is

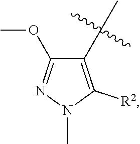

wherein R² is as described herein. In some embodiments, Ring A is

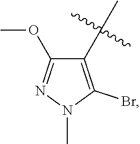 [SMILES: *c1c(OC)nn(C)c1Br]

In some embodiments, Ring A is

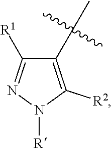

wherein each of R¹ and R² is independently optionally substituted C₁-C₆ alkyl and R is as described herein. In some embodiments, Ring A is

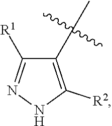

wherein each of R¹ and R² is independently optionally substituted C₁-C₆ alkyl. In some embodiments, Ring A is

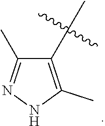

In some embodiments, Ring A is optionally substituted

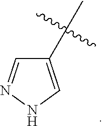 [SMILES: *c1cn[nH]c1]

### X¹

In some embodiments, X¹ is —N═, —C(R²)═ or optionally substituted —CH═ wherein each variable is independently as described herein. In some embodiments, X¹ is —N═. In some embodiments, X¹ is —C(R²)═ wherein R² is as described herein. In some embodiments, R² is R¹ described herein. In some embodiments, R² is R as described herein. In some embodiments, X¹ is optionally substituted —CH═. In some embodiments, X¹ is —CH═.

### X^{2a}

In some embodiments, X^{2a} is —N═, —C(R²)═ or optionally substituted —CH═ wherein each variable is independently as described herein. In some embodiments, X^{2a} is —N═. In some embodiments, X^{2a} is —C(R²)═ wherein R² is as described herein. In some embodiments, R² is R¹ described herein. In some embodiments, R² is R described herein. In some embodiments, X^{2a} is optionally substituted —C(H)═. In some embodiments, X^{2a} is —C(H)═. In some embodiments, X^{2a} is —C(OR′)═ wherein R′ is as described herein. In some embodiments, X^{2a} is —C(OH)═. In some embodiments, X^{2a} is —C(OR′)═ wherein R′ is optionally substituted C₁-C₆ alkyl. In some embodiments, X^{2a} is —C(OMe)=. In some embodiments, X^{2a} is —C(N(R′)₂)═. In some embodiments, X^{2a} is —C(NH₂)═. In some embodiments, X^{2a} is —C(N(R′)₂)═ wherein each R′ is independently optionally substituted C₁-C₆ alkyl. In some embodiments, X^{2a} is —C(R²)═ wherein R² is optionally substituted C₁-C₆ alkyl. In some embodiments, X^{2a} is —C(Me)═. In some embodiments, X^{2b} is —C(halogen)═. In some embodiments, X^{2b} is —C(F)═. In some embodiments, X^{2b} is —C(Cl)═. In some embodiments, X^{2b} is —C(Br)═. In some embodiments, X^{2b} is —C(I)═.

### X^{2b}

In some embodiments, X^{2b} is —N═, —C(R²)═ or optionally substituted CH═ wherein each variable is independently as described herein. In some embodiments, X^{2b} is —N═. In some embodiments, X^{2b} is —C(R²)═ wherein R² is as described herein. In some embodiments, R² is R′ described herein. In some embodiments, R² is R described herein. In some embodiments, X^{2b} is optionally substituted —C(H)═. In some embodiments, X^{2b} is —C(H)═. In some embodiments, X^{2b} is —C(OR′)═ wherein R′ is as described herein. In some embodiments, X^{2b} is —C(OH)═. In some embodiments, X^{2b} is —C(OR′)═ wherein R′ is optionally substituted C₁-C₆ alkyl. In some embodiments, X^{2b} is —C(OMe)=. In some embodiments, X^{2b} is —C(N(R′)₂)═. In some embodiments, X^{2b} is —C(NH₂)═. In some embodiments, X^{2b} is —C(N(R′)₂)═ wherein each R′ is independently optionally substituted C₁-C₆ alkyl. In some embodiments, X^{2b} is —C(R²)═ wherein R² is optionally substituted C₁-C₆ alkyl. In some embodiments, X^{2b} is —C(Me)═. In some embodiments, X^{2b} is —C(halogen)═. In some embodiments, X^{2b} is —C(F)═. In some embodiments, X^{2b} is —C(Cl)═. In some embodiments, X^{2b} is —C(Br)═. In some embodiments, X^{2b} is —C(I)═.

### X^{2c}

In some embodiments, X^{2c} is —N═, —C(R²)═ or optionally substituted —CH═ wherein each variable is independently as described herein. In some embodiments, X^{2c} is —N═. In some embodiments, X^{2c} is —C(R²)═ wherein R² is as described herein. In some embodiments, R² is R¹ described herein. In some embodiments, R² is R described herein. In some embodiments, X^{2c} is optionally substituted —C(H)═. In some embodiments, X^{2c} is —C(H)═. In some embodiments, X^{2c} is —C(OR′)═ wherein R′ is as described herein. In some embodiments, X^{2c} is —C(OH)═. In some embodiments, X^{2c} is —C(OR′)═ wherein R′ is optionally substituted C₁-C₆ aliphatic. In some embodiments, X^{2c} is —C(OR′)═ wherein R′ is C₁-C₆ aliphatic. In some embodiments, X^{2c} is —C(OR′)═ wherein R′ is optionally substituted C₁-C₆ alkyl. In some embodiments, X^{2b} is —C(OMe)═. In some embodiments, X^{2c} is

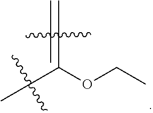 [SMILES: *C(=C)OCC]

In some embodiments, X^{2c} is

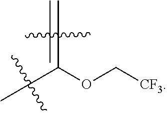 [SMILES: *C(=C)OCC(F)(F)F]

In some embodiments, X^{2c} is

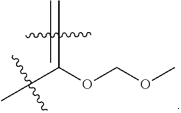 [SMILES: *C(=C)OCOC]

In some embodiments, X^{2c} is

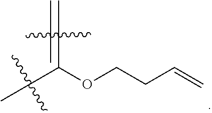 [SMILES: *C(=C)OCCC=C]

In some embodiments, X^{2c} is

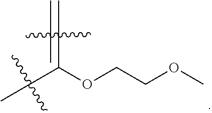 [SMILES: *C(=C)OCCOC]

In some embodiments, X^{2c} is

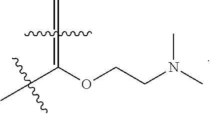 [SMILES: *C(=*)OCCN(C)C]

In some embodiments, X^{2c} is —C(N(R′)₂)═. In some embodiments, X^{2c} is —C(N(R′)₂)═ wherein each R′ is independently optionally substituted C₁-C₆ alkyl. In some embodiments, X^{2c} is

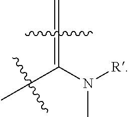

In some embodiments, X^{2c} is —C(N(R′)₂)═ wherein one R′ is taken together with another R group (e.g., R³ when it is R) to form an optionally substituted ring as described herein. In some embodiments, X^{2c} is

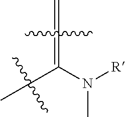

wherein R′ and another R group on another atom (e.g., R³ which can be R) are taken together with their intervening atoms to form an optionally substituted 3-10 membered ring having, in addition to the intervening atoms, 0-4 heteroatoms as described herein. In some embodiments, the present disclosure provides a compound having a structure of

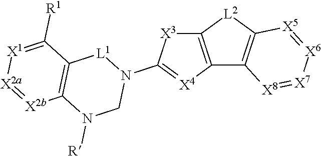

or a salt thereof, wherein each variable is independently as described herein. In some embodiments, the present disclosure provides a compound having a structure of

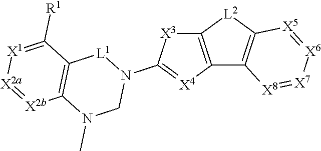

or a salt thereof, wherein each variable is independently as described herein. In some embodiments, X^{2c} is —C(NH₂)═.

In some embodiments, X^{2c} is —C(R²)═ wherein R² is optionally substituted C₁-C₆ aliphatic. In some embodiments, X^{2c} is —C(R²)═ wherein R² is C₁-C₆ aliphatic. In some embodiments, X^{2c} is —C(R²)═ wherein R² is optionally substituted C₁-C₆ alkyl. In some embodiments, X^{2c} is —C(Me)═. In some embodiments, X^{2c} is

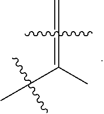 [SMILES: *C(=*)C]

In some embodiments, X^{2c} is

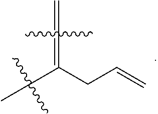 [SMILES: *C(=*)CC=C]

In some embodiments, X^{2c} is —C(halogen)=. In some embodiments, X^{2c} is —C(F)═. In some embodiments, X^{2c} is —C(Cl)═. In some embodiments, X^{2c} is —C(Br)═. In some embodiments, X^{2c} is —C(I)═.

### X^{1a}

In some embodiments, X^{1a} is —O—, —S—, —N(R′)—, —C(R′)₂—, or optionally substituted —CH₂— or —NH—, wherein R′ is as described herein. In some embodiments, X^{1a} is —O—. In some embodiments, X^{1a} is —S—. In some embodiments, X^{1a} is —N(R′)— wherein R′ is as described herein. In some embodiments, X^{1a} is —N(H)—. In some embodiments, X^{1a} is —N(R′)— wherein R′ is optionally substituted C₁-C₆ aliphatic. In some embodiments, X^{1a} is —N(R′)— wherein R′ is C₁-C₆ aliphatic. In some embodiments, X^{1a} is —N(R′)— wherein R′ is optionally substituted C₁-C₆ alkyl. In some embodiments, X^{1a} is —N(Me)-. In some embodiments, X^{1a} is —C(R′)₂— and each R′ is independently as described herein. In some embodiments, X^{1a} is optionally substituted —CH₂—. In some embodiments, X^{1a} is —CH₂—. In some embodiments, X^{1a} is optionally substituted —NH—. In some embodiments, X^{1a} is —NH—.

### X³

In some embodiments, X³ is —O—, —S—, —Se—, —N(R′)— or optionally substituted —NH— wherein R′ is as described herein. In some embodiments, X³ is —O—. In some embodiments, X³ is —S—. In some embodiments, X³ is —Se—. In some embodiments, X³ is —N(R′)— wherein R′ is as described herein. In some embodiments, X³ is —N(R′)— wherein R′ is optionally substituted C₁-C₆ aliphatic. In some embodiments, X³ is —N(R′)— wherein R′ is C₁-C₆ aliphatic. In some embodiments, X³ is —N(R′)— wherein R′ is optionally substituted C₁-C₆ alkyl. In some embodiments, X³ is —N(Me)-. In some embodiments, X³ is optionally substituted —NH—. In some embodiments, X³ is —NH—.

### X⁴

In some embodiments, X⁴ is —N═, —C(R⁶)═ or optionally substituted —CH═ wherein R⁶ is as described herein. In some embodiments, X⁴ is —N═. In some embodiments, X⁴ is —C(R⁶)═ wherein R⁶ is as described herein. In some embodiments, R⁶ is R¹ as described herein. In some embodiments, R⁶ is R as described herein. In some embodiments, X⁴ is optionally substituted —CH═. In some embodiments, X⁴ is —CH═.

### X⁵

In some embodiments, X⁵ is —N═, —C(R⁶)═ or optionally substituted —CH═ wherein R⁶ is as described herein. In some embodiments, X⁵ is —N═. In some embodiments, X⁵ is —C(R⁶)═ wherein R⁶ is as described herein. In some embodiments, R⁶ is R¹ as described herein. In some embodiments, R⁶ is R as described herein. In some embodiments, X⁵ is optionally substituted —CH═. In some embodiments, X⁵ is —CH═. In some embodiments, X⁵ is —C(R⁶)═ wherein R⁶ is optionally substituted C₁-C₆ aliphatic. In some embodiments, X⁵ is —C(R⁶)═ wherein R⁶ is C₁-C₆ aliphatic. In some embodiments, X⁵ is —C(R⁶)═ wherein R⁶ is optionally substituted C₁-C₆ alkyl. In some embodiments, X⁵ is —C(CF₃)═.

### X⁶

In some embodiments, X⁶ is —N═, —C(R⁶)═ or optionally substituted —CH═ wherein R⁶ is as described herein. In some embodiments, X⁶ is —N═. In some embodiments, X⁶ is —C(R⁶)═ wherein R⁶ is as described herein. In some embodiments, R⁶ is R¹ as described herein. In some embodiments, R⁶ is R as described herein. In some embodiments, X⁶ is optionally substituted —CH═. In some embodiments, X⁶ is —CH═. In some embodiments, X⁶ is —C(OH)═. In some embodiments, X⁶ is —C(OR′)═ wherein R′ is optionally substituted C₁-C₆ aliphatic. In some embodiments, X⁶ is —C(OR′)═ wherein R′ is C₁-C₆ aliphatic. In some embodiments, X⁶ is —C(OR′)═ wherein R′ is optionally substituted C₁-C₆ alkyl. In some embodiments, X⁶ is —C(OR′)═ wherein R′ is halogen substituted C₁-C₆ alkyl. In some embodiments, X⁶ is —C(OR′)═ wherein R′ is substituted C₁-C₆ alkyl and each substituent is —F. In some embodiments, X⁶ is —C(OMe)═. In some embodiments, X⁶ is —C(OCF₃)═. In some embodiments, X⁶ is —C(OCHF₂)═. In some embodiments, X⁶ is —C(OCH₂F)═. In some embodiments, X⁶ is —C(OCF₂Cl)═. In some embodiments, X⁶ is —C(OR′)═ wherein R′ is optionally substituted C₁-C₆ alkyl and R′ is taken together with another R′ group on another atom and their intervening atoms to form an optionally substituted 3-10 membered ring having, in addition to the intervening atoms, 0-4 heteroatoms. In some embodiments, both X⁶ and X⁷ are —C(OR′)— and the two R′ are taken together with their intervening atoms to form an optionally substituted 4-10 (e.g., 5-10, 5-9, 5-6, etc.) membered ring having, in addition to the intervening atoms, 0-3 heteroatoms. In some embodiments, both X⁶ and X⁷ are —C(R⁶)— and the two R⁶ are taken together with their intervening atoms to form an optionally substituted 4-10 (e.g., 5-10, 5-9, 5-6, etc.) membered ring having, in addition to the intervening atoms, 0-4 (e.g., 0, 1, 2, 3 or 4) heteroatoms. In some embodiments, the present disclosure provides a compound having a structure of

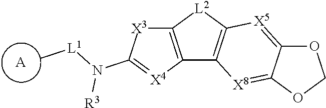

or a salt thereof.

In some embodiments, X⁶ is —C(OR′)═ wherein R′ is —C(O)R and R is as described herein. In some embodiments, X⁶ is —C(OR′)═ wherein R′ is —C(O)R and R is optionally substituted C₁-C₆ aliphatic. In some embodiments, X⁶ is

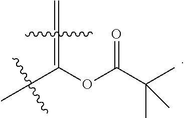 [SMILES: *C(=*)OC(=O)C(C)(C)C]

In some embodiments, X⁶ is —C(R⁶)═ wherein R⁶ is —C(O)OR′ and R′ is as described herein. In some embodiments, X⁶ is —C(R⁶)═ wherein R⁶ is —C(O)OR′ and R′ is optionally substituted C₁-C₆ aliphatic. In some embodiments, X⁶ is —C(R⁶)═ wherein R⁶ is —C(O)OCH₃. In some embodiments, X⁶ is —C(R⁶)═ wherein R⁶ is —C(O)OCH₂CH₃.

In some embodiments, X⁶ is —C(R⁶)═ wherein R⁶ is —C(O)R and R is as described herein. In some embodiments, X⁶ is —C(R⁶)═ wherein R⁶ is —C(O)R and R is optionally substituted C₁-C₆ aliphatic. In some embodiments, X⁶ is —C(R⁶)═ wherein R⁶ is —C(O)CH₃.

In some embodiments, X⁶ is —C(R⁶)═ wherein R⁶ is —CN. In some embodiments, X⁶ is —C(R⁶)═ wherein R⁶ is —NO₂. In some embodiments, X⁶ is —C(R⁶)═ wherein R⁶ is —N(R′)₂ and R′ is as described herein. In some embodiments, X⁶ is —C(R⁶)═ wherein R⁶ is —N(R′)₂ and each R′ is independently optionally substituted C₁-C₆ aliphatic. In some embodiments, X⁶ is —C(R⁶)═ wherein R⁶ is —N(R′)₂ and each R′ is independently C₁-C₆ aliphatic. In some embodiments, X⁶ is —C(R⁶)═ wherein R⁶ is —N(R′)₂ and each R′ is independently optionally substituted C₁-C₆ alkyl. In some embodiments, X⁶ is —C(R⁶)═ wherein R⁶ is —N(R′)₂ and two R′ are each independently as described herein and are taken together with the intervening nitrogen atom to form an optionally substituted 3-10 membered ring having, in addition to the nitrogen atom, 0-4 heteroatoms. In some embodiments, X⁶ is —C(R⁶)═ wherein R⁶ is —N(R′)₂ and two R′ are each independently as described herein are taken together with the intervening nitrogen atom to form an optionally substituted 3-6 membered ring having, in addition to the nitrogen atom, 0 heteroatoms. In some embodiments, X⁶ is —C(R⁶)═ wherein R⁶ is —N(R′)₂ and two R′ are each independently as described herein are taken together with the intervening nitrogen atom to form an optionally substituted 3-6 membered ring having, in addition to the nitrogen atom, 1 heteroatom. In some embodiments, X⁶ is —C(R⁶)═ wherein R⁶ is —N(R′)₂ and two R′ are each independently as described herein are taken together with the intervening nitrogen atom to form an optionally substituted 3-6 membered ring having, in addition to the nitrogen atom, 2 heteroatoms. In some embodiments, X⁶ is —C(R⁶)═ wherein R⁶ is

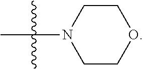 [SMILES: *N1CCOCC1]

In some embodiments, X⁶ is —C(R⁶)═ wherein R⁶ is optionally substituted C₁-C₁₀ aliphatic. In some embodiments, X⁶ is —C(R⁶)═ wherein R⁶ is optionally substituted C₁-C₆ aliphatic. In some embodiments, X⁶ is —C(R⁶)═ wherein R⁶ is C₁-C₆ aliphatic. In some embodiments, X⁶ is —C(R⁶)═ wherein R⁶ is optionally substituted C₁-C₆ alkyl. In some embodiments, X⁶ is —C(R⁶)═ wherein R⁶ is halogen substituted C₁-C₆ alkyl. In some embodiments, X⁶ is —C(R⁶)═ wherein R⁶ is substituted C₁-C₆ alkyl and each substituent is —F. In some embodiments, X⁶ is —C(R⁶)═ wherein R⁶ is C₁-C₆ alkyl. In some embodiments, X⁶ is —C(R⁶)═ wherein R⁶ is optionally substituted C₂-C₆ alkenyl. In some embodiments, X⁶ is —C(R⁶)═ wherein R⁶ is C₂-C₆ alkenyl. In some embodiments, X⁶ is —C(R⁶)═ wherein R⁶ is optionally substituted C₂-C₆ alkynyl. In some embodiments, X⁶ is —C(R⁶)═ wherein R⁶ is C₂-C₆ alkynyl. In some embodiments, X⁶ is —C(R⁶)═ wherein R⁶ is methyl. In some embodiments, X⁶ is —C(R⁶)═ wherein R⁶ is ethyl. In some embodiments, X⁶ is —C(R⁶)═ wherein R⁶ is —CF₃. In some embodiments, X⁶ is —C(R⁶)═ wherein R⁶ is —CH₂F. In some embodiments, X⁶ is —C(R⁶)═ wherein R⁶ is

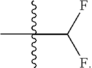 [SMILES: *C(F)F]

In some embodiments, X⁶ is —C(R⁶)═ wherein R⁶ is

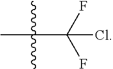 [SMILES: *C(F)(F)Cl]

In some embodiments, X⁶ is —C(R⁶)═ wherein R⁶ is

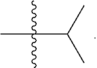 [SMILES: *C(C)C]

In some embodiments, X⁶ is —C(R⁶)═ wherein R⁶ is

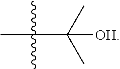 [SMILES: *C(C)(C)O]

In some embodiments, X⁶ is —C(R⁶)═ wherein R⁶ is

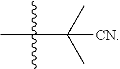 [SMILES: *C(C)(C)C#N]

In some embodiments, X⁶ is —C(R⁶)═ wherein R⁶ is

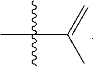 [SMILES: *C(=C)C]

In some embodiments, X⁶ is —C(R⁶)═ wherein R⁶ is

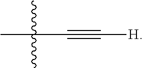

In some embodiments, X⁶ is —C(R⁶)═ wherein R⁶ is optionally substituted 3-10 membered cycloaliphatic. In some embodiments, X⁶ is —C(R⁶)═ wherein R⁶ is optionally substituted 3-6 membered cycloaliphatic. In some embodiments, X⁶ is —C(R⁶)═ wherein R⁶ is 3-6 membered cycloaliphatic. In some embodiments, X⁶ is —C(R⁶)═ wherein R⁶ is

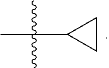 [SMILES: *C1CC1]

In some embodiments, X⁶ is —C(R⁶)═ wherein R⁶ is

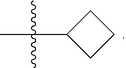 [SMILES: *C1CCC1]

In some embodiments, X⁶ is —C(R⁶)═ wherein R⁶ is

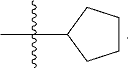 [SMILES: *C1CCCC1]

In some embodiments, X⁶ is —C(R⁶)═ wherein R⁶ is

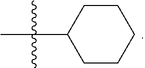 [SMILES: *C1CCCCC1]

In some embodiments, X⁶ is —C(R⁶)═ wherein R⁶ is halogen. In some embodiments, X⁶ is —C(F)═. In some embodiments, X⁶ is —C(Cl)═. In some embodiments, X⁶ is —C(Br)═. In some embodiments, X⁶ is —C(I)═.

### X⁷

In some embodiments, X⁷ is —N═, —C(R⁶)═ or optionally substituted —CH═ wherein R⁶ is as described herein. In some embodiments, X⁷ is —N═. In some embodiments, X⁷ is —C(R⁶)═ wherein R⁶ is as described herein. In some embodiments, R⁶ is R¹ as described herein. In some embodiments, R⁶ is R as described herein. In some embodiments, X⁷ is optionally substituted —CH═. In some embodiments, X⁷ is —CH═. In some embodiments, X⁷ is —C(OH)═. In some embodiments, X⁷ is —C(OR′)═ wherein R′ is optionally substituted C₁-C₆ aliphatic. In some embodiments, X⁷ is —C(OR′)═ wherein R′ is C₁-C₆ aliphatic. In some embodiments, X⁷ is —C(OR′)═ wherein R′ is optionally substituted C₁-C₆ alkyl. In some embodiments, X⁷ is —C(OR′)═ wherein R′ is halogen substituted C₁-C₆ alkyl. In some embodiments, X⁷ is —C(OR′)═ wherein R′ is substituted C₁-C₆ alkyl and each substituent is —F. In some embodiments, X⁷ is —C(OMe)═. In some embodiments, X⁷ is —C(OCF₃)═. In some embodiments, X⁷ is —C(OCHF₂)═. In some embodiments, X⁷ is —C(OCH₂F)═. In some embodiments, X⁷ is —C(OCF₂Cl)═. In some embodiments, X⁷ is —C(OR′)═ wherein R′ is optionally substituted C₁-C₆ alkyl and R′ is taken together with another R′ group on another atom and their intervening atoms to form an optionally substituted 3-10 membered ring having, in addition to the intervening atoms, 0-4 heteroatoms.

In some embodiments, X⁷ is —C(R⁶)═ wherein R⁶ is optionally substituted C₁-C₁₀ aliphatic. In some embodiments, X⁷ is —C(R⁶)═ wherein R⁶ is optionally substituted C₁-C₆ aliphatic. In some embodiments, X⁷ is —C(R⁶)═ wherein R⁶ is C₁-C₆ aliphatic. In some embodiments, X⁷ is —C(R⁶)═ wherein R⁶ is optionally substituted C₁-C₆ alkyl. In some embodiments, X⁷—C(R⁶)═ wherein R⁶ is halogen substituted C₁-C₆ alkyl. In some embodiments, X⁷ is —C(R⁶)═ wherein R⁶ is substituted C₁-C₆ alkyl and each substituent is —F. In some embodiments, X⁷ is —C(R⁶)═ wherein R⁶ s C₁-C₆ alkyl. In some embodiments, X⁷ is —C(R⁶)═ wherein R⁶ is optionally substituted C₂-C₆ alkenyl. In some embodiments, X⁷ is —C(R⁶)═ wherein R⁶ is C₂-C₆ alkenyl. In some embodiments, X⁷ is —C(R⁶)═ wherein R⁶ is optionally substituted C₂-C₆ alkynyl. In some embodiments, X⁷ is —C(R⁶)═ wherein R⁶ is C₂-C₆ alkynyl. In some embodiments, X⁷ is —C(R⁶)═ wherein R⁶ is methyl. In some embodiments, X⁷ is —C(R⁶)═ wherein R⁶ is ethyl. In some embodiments, X⁷ is —C(R⁶)═ wherein R⁶ is —CF₃. In some embodiments, X⁷ is —C(R⁶)═ wherein R⁶ is —CH₂F. In some embodiments, X⁷ is —C(R⁶)═ wherein R⁶ is —CHF₂. In some embodiments, X⁷ is —C(R⁶)═ wherein R⁶ is —CF₂Cl.

In some embodiments, X⁷ is —C(R⁶)═ wherein R⁶ is halogen. In some embodiments, X⁷ is —C(F)═. In some embodiments, X⁷ is —C(Cl)═. In some embodiments, X⁷ is —C(Br)═. In some embodiments, X⁷ is —C(I)═.

In some embodiments, X⁸ is —N═, —C(R⁶)═ or optionally substituted —CH═ wherein R⁶ is as described herein. In some embodiments, X⁸ is —N═. In some embodiments, X⁸ is —C(R⁶)═ wherein R⁶ is as described herein. In some embodiments, R⁶ is R¹ as described herein. In some embodiments, R⁶ is R as described herein. In some embodiments, X⁸ is optionally substituted —CH═. In some embodiments, X⁸ is —CH═. In some embodiments, X³ is —C(OH)═. In some embodiments, X⁸ is —C(OR′)═ wherein R′ is optionally substituted C₁-C₆ aliphatic. In some embodiments, X⁸ is —C(OR′)═ wherein R′ is C₁-C₆ aliphatic. In some embodiments, X⁸ is —C(OR′)═ wherein R′ is optionally substituted C₁-C₆ alkyl. In some embodiments, X⁸ is —C(OR′)═ wherein R′ is halogen substituted C₁-C₆ alkyl. In some embodiments, X⁸ is —C(OR′)═ wherein R′ is substituted C₁-C₆ alkyl and each substituent is —F. In some embodiments, X⁸ is —C(OMe)═. In some embodiments, X⁸ is —C(OCF₃)═. In some embodiments, X⁸ is —C(OCHF₂)═. In some embodiments, X⁸ is —C(OCH₂F)═. In some embodiments, X⁸ is —C(OCF₂Cl)═. In some embodiments, X⁸ is —C(OR′)═ wherein R′ is optionally substituted C₁-C₆ alkyl and R′ is taken together with another R′ group on another atom and their intervening atoms to form an optionally substituted 3-10 membered ring having, in addition to the intervening atoms, 0-4 heteroatoms. In some embodiments, both X⁷ and X⁸ are —C(OR′)— and the two R′ are taken together with their intervening atoms to form an optionally substituted 4-10 (e.g., 5-10, 5-9, 5-6, etc.) membered ring having, in addition to the intervening atoms, 0-3 heteroatoms. In some embodiments, both X⁷ and X⁸ are —C(R⁶)— and the two R⁶ are taken together with their intervening atoms to form an optionally substituted 4-10 (e.g., 5-10, 5-9, 5-6, etc.) membered ring having, in addition to the intervening atoms, 0-3 heteroatoms. In some embodiments, the present disclosure provides a compound having a structure of

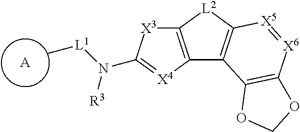

or a salt thereof.

In some embodiments, X⁸ is —C(R⁶)═ wherein R⁶ is optionally substituted C₁-C₁₀ aliphatic. In some embodiments, X⁸ is —C(R⁶)═ wherein R⁶ is optionally substituted C₁-C₆ aliphatic. In some embodiments, X⁸ is —C(R⁶)═ wherein R⁶ is C₁-C₆ aliphatic. In some embodiments, X⁸ is —C(R⁶)═ wherein R⁶ is optionally substituted C₁-C₆ alkyl. In some embodiments, X⁸ is —C(R⁶)═ wherein R⁶ is halogen substituted C₁-C₆ alkyl. In some embodiments, X⁸ is —C(R⁶)═ wherein R⁶ is substituted C₁-C₆ alkyl and each substituent is —F. In some embodiments, X⁸ is —C(R⁶)═ wherein R⁶ is C₁-C₆ alkyl. In some embodiments, X⁸ is —C(R⁶)═ wherein R⁶ is optionally substituted C₂-C₆ alkenyl. In some embodiments, X⁸ is —C(R⁶)═ wherein R⁶ is C₂-C₆ alkenyl. In some embodiments, X⁸ is —C(R⁶)═ wherein R⁶ is optionally substituted C₂-C₆ alkynyl. In some embodiments, X³ is —C(R⁶)═ wherein R⁶ is C₂-C₆ alkynyl. In some embodiments, X⁸ is —C(R⁶)═ wherein R⁶ is methyl. In some embodiments, X⁸ is —C(R⁶)═ wherein R⁶ is ethyl. In some embodiments, X⁸ is —C(R⁶)═ wherein R⁶ is —CF₃. In some embodiments, X⁸ is —C(R⁶)═ wherein R⁶ is —CH₂F. In some embodiments, X⁸ is —C(R⁶)═ wherein R⁶ is

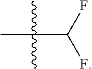 [SMILES: *C(F)F]

In some embodiments, X⁸ is —C(R⁶)═ wherein R⁶ is

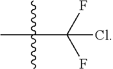 [SMILES: *C(F)(F)Cl]

In some embodiments, X⁸ is —C(R⁶)═ wherein R⁶ is halogen. In some embodiments, X⁸ is —C(F)═. In some embodiments, X⁸ is —C(Cl)═. In some embodiments, X⁸ is —C(Br)═. In some embodiments, X⁸ is —C(I)═.

### R¹

In some embodiments, R¹ is halogen, —CN, —NO₂, R′, —OR′, —N(R′)₂, —N(R′)C(O)OR′, —C(O)R′, —C(O)OR′, —C(O)N(R′)₂, or —OC(O)N(R′)₂, wherein R′ is independently as described herein. In some embodiments, R¹ is —H. In some embodiments, R¹ is not —H. In some embodiments, R¹ is —CN. In some embodiments, R¹ is —NO₂. In some embodiments, R¹ is R′ as described herein. In some embodiments, R¹ is -L-R′ wherein each L and R′ is independently as described herein. In some embodiments, L is optionally substituted C₁₋₃ alkylene. In some embodiments, R¹ is —CH₂R′ wherein R′ is as described herein. In some embodiments, R′ is —CH₂C(O)OR wherein R is as described herein. In some embodiments, R¹ is R as described herein. In some embodiments, R¹ is —N(R′)₂ wherein R′ is independently as described herein. In some embodiments, R¹ is —N(R′)C(O)OR′ wherein each R′ is independently as described herein. In some embodiments, R¹ is —C(O)R′ wherein R′ is as described herein. In some embodiments, R′ is —C(O)OR′ wherein R′ is as described herein. In some embodiments, R′ is —C(O)N(R′)₂ wherein R′ is independently as described herein. In some embodiments, R′ is —OC(O)N(R′)₂ wherein R′ is independently as described herein.

In some embodiments, R′ is OR′ and R′ is as described herein. In some embodiments, R′ is —OH. In some embodiments, R¹ is —OR′ wherein R′ is optionally substituted C₁-C₆ aliphatic. In some embodiments, R¹ is —OR′ wherein R′ is C₁-C₆ aliphatic. In some embodiments, R¹ is —OR′ wherein R′ is optionally substituted C₁-C₆ alkyl. In some embodiments, R¹ is —OR′ wherein R′ is halogen substituted C₁-C₆ alkyl. In some embodiments, R¹ is —OR′ wherein R′ is substituted C₁-C₆ alkyl and each substituent is —F. In some embodiments, R¹ is —OMe. In some embodiments, R¹ is —OCF₃. In some embodiments, R¹ is —OCHF₂. In some embodiments, R¹ is —OCH₂F. In some embodiments, R¹ is —OCF₂Cl. In some embodiments, R¹ is

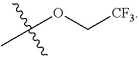 [SMILES: *OCC(F)(F)F]

In some embodiments, R¹ is optionally substituted C₁-C₁₀ aliphatic. In some embodiments, R¹ is optionally substituted C₁-C₆ aliphatic. In some embodiments, R¹ is C₁-C₆ aliphatic. In some embodiments, R¹ is optionally substituted C₁-C₆ alkyl. In some embodiments, R¹ is halogen substituted C₁-C₆ alkyl. In some embodiments, R¹ is substituted C₁-C₆ alkyl and each substituent is —F. In some embodiments, R¹ is C₁-C₆ alkyl. In some embodiments, R¹ is optionally substituted C₂-C₆ alkenyl. In some embodiments, R¹ is C₂-C₆ alkenyl. In some embodiments, R¹ is optionally substituted C₂-C₆ alkynyl. In some embodiments, R¹ is C₂-C₆ alkynyl. In some embodiments, R¹ is methyl. In some embodiments, R¹ is ethyl. In some embodiments, R¹ is —CF₃. In some embodiments, R¹ is —CH₂F. In some embodiments, R¹ is

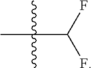 [SMILES: *C(F)F]

In some embodiments, R¹ is

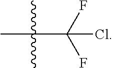 [SMILES: *C(F)(F)Cl]

In some embodiments, R¹ is halogen. In some embodiments, R¹ is F. In some embodiments, R¹ is Cl. In some embodiments, R¹ is Br. In some embodiments, R¹ is I.

### R²

In some embodiments, R² is R¹ as described herein. In some embodiments, R² is R′ as described herein. In some embodiments, R² is R as described herein. In some embodiments, R² is —H. In some embodiments, R² is not —H. In some embodiments, R² is optionally substituted C₁₋₆ aliphatic. In some embodiments, R² is optionally substituted C₁₋₆ alkyl. In some embodiments, R² is optionally substituted C₁-C₁₀ (e.g., C₁, C₂, C₃, C₄, C₅, C₆, C₇, C₈, C₉, or C₁₀) heteroaliphatic having 1-3 (e.g., 1, 2, or 3) heteroatoms. In some embodiments, there is one heteroatom. In some embodiments, there are two heteroatoms. In some embodiments, there are three heteroatoms. In some embodiments, a heteroatom is an oxygen atom. In some embodiments, at least two heteroatoms are different. In some embodiments, all hetereoatoms are the same.

In some embodiments, R² is —OR′ wherein R′ is as described herein. In some embodiments, R² is —OH. In some embodiments, R² is —OR′ wherein R′ is optionally substituted C₁₋₆ aliphatic.

In some embodiments, R² and another R group (e.g., another variable such as R², R³, etc. when it is R) are taken together with their intervening atom(s) to form an optionally substituted ring as described herein. In some embodiments, a formed ring is an optionally substituted 3-10, 3-9, 3, 4, 5, 6, 7, 8, 9, or 10 membered ring having, in addition to the intervening atoms, 0-4 heteroatoms.

### R³

In some embodiments, R³ is R′ wherein R′ is as described herein. In some embodiments, R³ is R as described herein. In some embodiments, R³ is H. In some embodiments, R³ is optionally substituted C₁-C₆ alkyl. In some embodiments, R³ is C₁-C₆ alkyl. In some embodiments, R³ is methyl. In some embodiments, R³ is ethyl.

### R⁴

In some embodiments, R⁴ is R′ wherein R′ is as described herein. In some embodiments, R⁴ is R as described herein. In some embodiments, R⁴ is H. In some embodiments, R⁴ is optionally substituted C₁-C₆ aliphatic. In some embodiments, R⁴ is optionally substituted C₁-C₆ alkyl. In some embodiments, R⁴ is methyl. In some embodiments, R⁴ is —CH₂F. In some embodiments, R⁴ is —CH₂OH. In some embodiments, R⁴ is ethyl. In some embodiments, R⁴ is n-hexyl. In some embodiments, R⁴ is —(CH₂)₂CH═CH₂. In some embodiments, R⁴ is —(CH₂)₃COOH. In some embodiments, R⁴ is optionally substituted C₁-C₁₀ (e.g., C₁, C₂, C₃, C₄, C₅, C₆, C₇, C₈, Co, or C₁₀) heteroaliphatic having 1-3 (e.g., 1, 2, or 3) heteroatoms. In some embodiments, R⁴ is —CH₂OCH₃. In some embodiments, R⁴ is —CH₂OCH₂OCH₃. In some embodiments, R⁴ is optionally substituted C₆-C₁₀ aryl-C₁₋₁₀ aliphatic. For example, in some embodiments, R⁴ is —(CH₂)₂Ph. In some embodiments, R⁴ and R⁵ are both R and are taken together with their intervening atom(s) to form an optionally substituted ring as described herein.

### R⁵

In some embodiments, R⁵ is R′ wherein R′ is as described herein. In some embodiments, R⁵ is R as described herein. In some embodiments, R⁵ is H. In some embodiments, R⁵ is optionally substituted C₁-C₆ aliphatic. In some embodiments, R⁵ is optionally substituted C₁-C₆ alkyl. In some embodiments, R⁵ is methyl. In some embodiments, R⁵ is —CH₂F. In some embodiments, R⁵ is —CH₂OH. In some embodiments, R⁵ is ethyl. In some embodiments, R⁵ is n-hexyl. In some embodiments, R⁵ is —(CH₂)₂CH═CH₂. In some embodiments, R⁵ is —(CH₂)₃COOH. In some embodiments, R⁵ is optionally substituted C₁-C₁₀ (e.g., C₁, C₂, C₃, C₄, C₅, C₆, C₇, C₈, C₉, or C₁₀) heteroaliphatic having 1-3 (e.g., 1, 2, or 3) heteroatoms. In some embodiments, R⁵ is —CH₂OCH₃. In some embodiments, R⁵ is —CH₂OCH₂OCH₃. In some embodiments, R⁵ is optionally substituted C₆-C₁₀ aryl-C₁₋₁₀ aliphatic. For example, in some embodiments, R⁵ is —(CH₂)₂Ph. In some embodiments, R⁴ and R⁵ are both R and are taken together with their intervening atom(s) to form an optionally substituted ring as described herein.

### R⁶

In some embodiments, R⁶ is R¹ as described herein. In some embodiments, R⁶ is R′ as described herein. In some embodiments, R⁶ is R as described herein. In some embodiments, R⁶ and another group that can be R are taken together with their intervening atom(s) to form an optionally substituted ring as described herein. In some embodiments, two R⁶ (e.g., of X⁶ and X⁷ when each of them is independently —C(R⁶)═) are R, and are taken together with their intervening atom(s) to form an optionally substituted ring as described herein. In some embodiments, R⁶ is —H. In some embodiments, R⁶ is not —H.

In some embodiments, R⁶ is halogen. In some embodiments, R⁶ is —F. In some embodiments, R⁶ is —Cl. In some embodiments, R⁶ is —Br. In some embodiments, R⁶ is —I. In some embodiments, R⁶ is —CN. In some embodiments, R⁶ is —NO₂. In some embodiments, R⁶ is R′ as described herein. In some embodiments, R⁶ is —OR′ as described herein. In some embodiments, R⁶ is —OH. In some embodiments, R⁶ is —OR′ wherein R′ is optionally substituted C₁₋₆ aliphatic. In some embodiments, R⁶ is —OR′ wherein R′ is C₁₋₆ aliphatic. In some embodiments, R⁶ is —OR′ wherein R′ is optionally substituted C₁₋₆ alkyl. In some embodiments, R⁶ is —OR′ wherein R′ is C₁₋₆ alkyl. In some embodiments, R⁶ is —OR′ wherein R′ is C₁₋₆ haloalkyl. In some embodiments, R⁶ is —OR′ wherein R′ is substituted C₁₋₆ alkyl wherein each substituent is —F. In some embodiments, R⁶ is —OMe. In some embodiments, R⁶ is —OCF₃. In some embodiments, R⁶ is —OCHF₂. In some embodiments, R⁶ is —OCH₂F. In some embodiments, R⁶ is —OCF₂Cl. In some embodiments, X⁶ is —OR′ wherein R′ is taken together with another R′ group on another atom and their intervening atoms to form an optionally substituted 3-10 membered ring having, in addition to the intervening atoms, 0-4 heteroatoms. In some embodiments, a provided compound, e.g., a compound having the structure of formula I or a salt thereof has the structure of

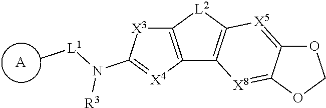

or a salt thereof. In some embodiments, R⁶ is —OR′ wherein R′ is —C(O)R wherein R is as described herein.

In some embodiments, R⁶ is —N(R′)₂ wherein each R′ is independently as described herein. In some embodiments, the two R′ are taken together with the nitrogen to which they are attached to form an optionally substituted ring as described herein. In some embodiments, one of the R′ and another R attached to another atom are taken together with their intervening atoms to form an optionally substituted ring as described herein. In some embodiments, R⁶ is —N(R′)C(O)OR′ wherein each R′ is independently as described herein. In some embodiments, R⁶ is —C(O)R′ wherein R′ is as described herein. In some embodiments, R⁶ is —C(O)R′ wherein R′ is optionally substituted C₁₋₆ aliphatic. In some embodiments, R⁶ is —C(O)CH₃. In some embodiments, R⁶ is —C(O)OR′ wherein R′ is as described herein. In some embodiments, R⁶ is —C(O)OR′ wherein R′ is optionally substituted C₁₋₆ aliphatic. In some embodiments, R⁶ is —C(O)OCH₃. In some embodiments, R⁶ is —C(O)OCH₂CH₃. In some embodiments, R⁶ is —C(O)N(R′)₂ wherein each R′ is independently as described herein. In some embodiments, R⁶ is —OC(O)N(R′)₂ wherein each R′ is independently as described herein.

In some embodiments, R⁶ is optionally substituted C₁-C₁₀ aliphatic. In some embodiments, R⁶ is optionally substituted C₁₋₆ aliphatic (as those skilled in the art appreciate, C₁₋₆ (e.g., C₁₋₆) may alternatively be presented as C₁-C₆ (e.g., C₁-C₆). In some embodiments, R⁶ is C₁₋₆ aliphatic. In some embodiments, R⁶ is optionally substituted C₁₋₆ alkyl. In some embodiments, R⁶ is C₁₋₆ alkyl. In some embodiments, R⁶ is C₁-C₆ haloalkyl. In some embodiments, R⁶ is C₁-C₆ aliphatic substituted with one or more-F. In some embodiments, R⁶ is C₁-C₆ alkyl substituted with one or more-F. In some embodiments, R⁶ is optionally substituted C₂-C₆ alkenyl. In some embodiments, R⁶ is optionally substituted C₂-C₆ alkynyl. In some embodiments, R⁶ is methyl. In some embodiments, R⁶ is ethyl. In some embodiments, R⁶ is —CF₃. In some embodiments, R⁶ is —CH₂F. In some embodiments, cr6 is —CHF₂. In some embodiments, R⁶ is —CF₂Cl. In some embodiments, R⁶ is —CH(CH₃)₂. In some embodiments, R⁶ is —C(CH₃)₂OH. In some embodiments, R⁶ is —C(CH₃)₂CN. In some embodiments, R⁶ is —C(CH₃)—CH₂. In some embodiments, R⁶ is —C═CH. In some embodiments, R⁶ is optionally substituted 3-10 (e.g., 3, 4, 5, 6, 7, 8, 9, or 10) membered cycloaliphatic. In some embodiments, R⁶ is optionally substituted 3-10 (e.g., 3, 4, 5, 6, 7, 8, 9, or 10) membered cycloalkyl. In some embodiments, R⁶ is optionally substituted cyclopropyl. In some embodiments, R⁶ is optionally substituted cyclobutyl. In some embodiments, R⁶ is optionally substituted cyclopentyl. In some embodiments, R⁶ is optionally substituted cyclohexyl.

### R⁵

In some embodiments, R^{s} is R¹ as described herein. In some embodiments, R⁵ is -L-R′ wherein each of L and R′ is independently as described herein. In some embodiments, L is optionally substituted —CH₂—. In some embodiments, L is —CH₂—. In some embodiments, R^{s} is R′ as described herein. In some embodiments, R^{s} is R as described herein. In some embodiments, R^{s} is —C(O)R′ wherein R′ is as described herein. In some embodiments, R^{s} is —C(O)R wherein R is as described herein. In some embodiments, R^{s} is —C(O)OR wherein R is as described herein. In some embodiments, R^{s} is —C(O)OtBu. In some embodiments, R^{s} is —CH₂C(O)OR wherein R is as described herein. In some embodiments, R^{s} is —CH₂C(O)OCH₃.

In some embodiments, two R^{s} are R, and are taken together with their intervening atom(s) to form an optionally substituted ring as described herein.

### R′

In some embodiments, R′ is R as described herein. In some embodiments, R′ is —OR wherein R is as described herein. In some embodiments, R′ is —C(O)R wherein R is as described herein. In some embodiments, R′ is —C(O)OR wherein R is as described herein. In some embodiments, R′ is —S(O)₂R wherein R is as described herein.

In some embodiments, R′ is —H. In some embodiments, R′ is not —H. In some embodiments, two R′ are R and are taken together with their intervening atom(s) to form a ring as described herein.

### R

In some embodiments, R is —H. In some embodiments, R is not —H.

In some embodiments, R is optionally substituted C₁-C₁₀ (e.g., C₁, C₂, C₃, C₄, C₅, C₆, C₇, C₈, C₉, or C₁₀) aliphatic. In some embodiments, R is optionally substituted C₁-C₁₀ (e.g., C₁, C₂, C₃, C₄, C₅, C₆, C₇, C₈, C₉, or C₁₀) alkyl. In some embodiments, R is optionally substituted C₁₋₆ aliphatic. In some embodiments, R is optionally substituted C₁₋₆ alkyl. For example, in some embodiments, R is optionally substituted hexyl. In some embodiments, R is hexyl. In some embodiments, R is optionally substituted pentyl. In some embodiments, R is pentyl. In some embodiments, R is optionally substituted butyl. In some embodiments, R is butyl. In some embodiments, R is optionally substituted propyl. In some embodiments, R is propyl. In some embodiments, R is isopropyl. In some embodiments, R is optionally substituted ethyl. In some embodiments, R is ethyl. In some embodiments, R is optionally substituted methyl. In some embodiments, R is methyl. In some embodiments, R is —CF₃.

In some embodiments, R is optionally substituted C₁-C₁₀ (e.g., C₁, C₂, C₃, C₄, C₅, C₆, C₇, C₈, C₉, or C₁₀) heteroaliphatic having 1-3 (e.g., 1, 2, or 3) heteroatoms. In some embodiments, R is optionally substituted C₁-C₁₀ (e.g., C₁, C₂, C₃, C₄, C₅, C₆, C₇, C₈, Co, or C₁₀) heteroalkyl having 1-3 (e.g., 1, 2, or 3) heteroatoms. In some embodiments, a heteroatom is oxygen. In some embodiments, a heteroatom is nitrogen. In some embodiments, a nitrogen atom is substituted. In some embodiments, a heteroatom is sulfur. In some embodiments, there is one heteroatom. In some embodiments, there are two heteroatoms. In some embodiments, there are three heteroatoms.

In some embodiments, R is optionally substituted 3-10 membered (e.g., 3, 4, 5, 6, 7, 8, 9, or 10) cycloaliphatic. In some embodiments, R is optionally substituted 3-10 membered (e.g., 3, 4, 5, 6, 7, 8, 9, or 10) cycloalkyl. In some embodiments, it is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, a ring is monocyclic. In some embodiments, a ring is bicyclic. In some embodiments, a ring is polycyclic. In some embodiments, R is optionally substituted cyclohexyl. In some embodiments, R is cyclohexyl. In some embodiments, R is optionally substituted cyclopentyl. In some embodiments, R is pentyl. In some embodiments, R is optionally substituted butyl. In some embodiments, R is butyl. In some embodiments, R is optionally substituted cyclopropyl. In some embodiments, R is cyclopropyl. In some embodiments, R is optionally substituted adamantyl. In some embodiments, R is adamantyl.

In some embodiments, R is optionally substituted 3-10 membered (e.g., 3, 4, 5, 6, 7, 8, 9, or 10) heterocyclyl having 1-4 (e.g., 1, 2, 3, or 4) heteroatoms. In some embodiments, a heterocyclyl ring is saturated. In some embodiments, it is partially unsaturated. In some embodiments, it is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, a ring is monocyclic. In some embodiments, a ring is bicyclic. In some embodiments, a ring is polycyclic. In some embodiments, a heteroatom is oxygen. In some embodiments, a heteroatom is nitrogen. In some embodiments, a nitrogen atom is substituted. In some embodiments, a heteroatom is sulfur. In some embodiments, there is one heteroatom. In some embodiments, there are two heteroatoms. In some embodiments, there are three heteroatoms.

In some embodiments, R is optionally substituted 6-10 membered aryl. In some embodiments, R is optionally substituted aryl. In some embodiments, R is optionally substituted phenyl. In some embodiments, R is phenyl. In some embodiments, R is optionally substituted 10-membered aryl. In some embodiments, R is optionally substituted naphthyl. In some embodiments, R is naphthyl.

In some embodiments, R is optionally substituted 5-10 (e.g., 5, 9 or 10) membered heteroaryl having 1-6 (e.g., 1, 2, 3, 4, 5, or 6) heteroatoms. In some embodiments, R is optionally substituted 5-membered heteroaryl having 1-4 (e.g., 1, 2, 3, or 4) heteroatoms. In some embodiments, R is optionally substituted 6-membered heteroaryl having 1-4 (e.g., 1, 2, 3, or 4) heteroatoms. In some embodiments, R is optionally substituted bicyclic 9-membered heteroaryl having 1-4 (e.g., 1, 2, 3, or 4) heteroatoms. In some embodiments, R is optionally substituted bicyclic 10-membered heteroaryl having 1-6 (e.g., 1, 2, 3, 4, 5 or 6) heteroatoms. In some embodiments, there is one heteroatom. In some embodiments, there are two heteroatoms. In some embodiments, there are three heteroatoms. In some embodiments, there are four heteroatoms. In some embodiments, a heteroatom is nitrogen. In some embodiments, a nitrogen atom is substituted. In some embodiments, a heteroatom is oxygen. In some embodiments, a heteroatom is sulfur. In some embodiments, each heteroatom is the same. In some embodiments, at least one heteroatom is different from at least one another.

In some embodiments, R is optionally substituted 6-10 (e.g., 6, 10, etc.) membered aryl-C₁-C₁₀ (e.g., C₁, C₂, C₃, C₄, C₅, C₆, C₇, C₈, C₉, or C₁₀) aliphatic. Certain embodiments of optionally substituted 6-10 membered aryl and optionally substituted C₁-C₁₀ aliphatic are described supra.

In some embodiments, R is optionally substituted 5-10 (e.g., 5, 6, 9, 10, etc.) membered heteroaryl having 1-6 (e.g., 1, 2, 3, 4, 5, or 6) heteroatoms-C₁-C₁₀ (e.g., C₁, C₂, C₃, C₄, C₅, C₆, C₇, C₈, C₉, or C₁₀) aliphatic. Certain embodiments of optionally substituted 5-10 membered heteroaryl having 1-6 heteroatoms and optionally substituted C₁-C₁₀ aliphatic are described supra.

In some embodiments, two R groups (in some embodiments, two groups that can be R, e.g., two groups each independently selected from R¹, R², R³, R⁴, R⁵, R⁶ and R^{s}) on the same atom are optionally and independently taken together with the atom to form an optionally substituted 3-10 (e.g., 3, 4, 5, 6, 7, 8, 9, or 10) membered ring having, in addition to the atom, 0-4 (0, 1, 2, 3, or 4) heteroatoms. In some embodiments, two R groups (in some embodiments, two groups that can be R, e.g., two groups each independently selected from R¹, R², R³, R⁴, R⁵, R⁶ and R^{s}) on two atoms are optionally and independently taken together with their intervening atoms to form an optionally substituted 3-10 (3, 4, 5, 6, 7, 8, 9, or 10) membered ring having, in addition to the intervening atoms, 0-4 (e.g., 1, 2, 3, or 4) heteroatoms.

In some embodiments, a ring of the present disclosure, e.g., formed by two groups taken together with their intervening atom(s), a cycloaliphatic ring, a heterocyclyl ring, an aryl ring, a heteroaryl ring, etc., is substituted. In some embodiments, it is unsubstituted. In some embodiments, it is saturated. In some embodiments, it is partially substituted. In some embodiments, it is aromatic. In some embodiments, there is a heteroatom in a ring. In some embodiments, there are two heteroatoms in a ring. In some embodiments, there are three heteroatoms in a ring. In some embodiments, there are four atoms in a ring. In some embodiments, there are no heteroatoms. In some embodiments, a ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is monocyclic. In some embodiments, it is bicyclic. In some embodiments, it is polycyclic. In some embodiments, each monocyclic ring unit is independently an optionally substituted 3-10, 3-9, 3-8, 3-7, 3-6, 3, 4, 5, 6, 7, 8, 9, or 10-membered saturated, partially unsaturated or aromatic ring independently having 0-4 (e.g., 0, 1, 2, 3, or 4) heteroatoms. In some embodiments, each monocyclic ring unit is independently an optionally substituted 3-7, 3, 4, 5, 6, or 7-membered saturated, partially unsaturated or aromatic ring independently having 0-4 (e.g., 0, 1, 2, 3, or 4) heteroatoms. In some embodiments, each monocyclic ring unit is independently an optionally substituted 3, 4, 5, or 6-membered saturated, partially unsaturated or aromatic ring independently having 0-4 (e.g., 0, 1, 2, 3, or 4) heteroatoms.

### L¹

In some embodiments, L¹ is a covalent bond, —C(O)—, —C(S)—, —S(O)₂—, —N(R′)C(O)—, —N(R′)S(O)₂—, —N(R′)C(S)— or

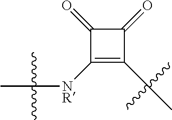

wherein each R′ is independently as described herein. In some embodiments, L¹ is covalent bond. In some embodiments, L¹ is —C(O)—. In some embodiments, L¹ is —C(S)—. In some embodiments, L¹ is —S(O)₂—. In some embodiments, L¹ is —N(R′)C(O)— wherein R′ is as described herein. In some embodiments, L¹ is —N(R′)C(O)— wherein R′ is optionally substituted C₁-C₆ alkyl. In some embodiments, L¹ is —N(H) C(O)—. In some embodiments, L¹ is —N(R′)S(O)₂— wherein R′ is as described herein. In some embodiments, L¹ is —N(R′)S(O)₂— wherein R′ is optionally substituted C₁-C₆ alkyl. In some embodiments, L¹ is —N(H)S(O)₂—. In some embodiments, L¹ is —N(R′)C(S)— wherein R′ is as described herein. In some embodiments, L¹ is —N(R′)C(S)— wherein R′ is optionally substituted C₁-C₆ alkyl. In some embodiments, L¹ is —N(H) C(S)—. In some embodiments, L¹ is

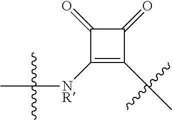

wherein R′ is as described herein. In some embodiments, R′ is —H. In some embodiments, R′ is optionally substituted C₁₋₆ alkyl.

### L

In some embodiments, L is optionally substituted C₁₋₃ alkylene, wherein one or more methylene unit of the alkylene is optionally and independently replaced with —O—, —S—, —C(R⁴)(R⁵)—, —N(R⁴)—, optionally substituted —CH═N—, optionally substituted —CH═CH— or -Cy-; and each of R⁴ and R⁵ is independently R′ as described herein; and each -Cy- is independently

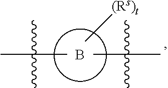

wherein Ring B is an optionally substituted saturated or partially saturated 3-10 membered ring having 0-4 heteroatoms; and t is 0, 1, 2, 3 or 4.

In some embodiments, L is optionally substituted C₁₋₃ alkylene. In some embodiments, L is optionally substituted —CH₂—. In some embodiments, L is —CH₂—. In some embodiments, L is optionally substituted —(CH₂)₂—. In some embodiments, L is —(CH₂)₂—. In some embodiments, L is optionally substituted —(CH₂)₃—. In some embodiments, L is —(CH₂)₃—.

In some embodiments, L is L² as described herein.

### L²

In some embodiments, L² is optionally substituted C₁₋₃ alkylene, wherein one or more methylene unit of the alkylene is optionally and independently replaced with —O—, —S—, —C(R⁴)(R⁵)—, —N(R⁴)—, optionally substituted —CH═N—, optionally substituted —CH═CH— or -Cy-; and each of R⁴ and R⁵ is independently R′ as described herein; and each -Cy- is independently

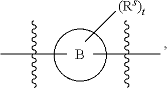

wherein Ring B is an optionally substituted saturated or partially saturated 3-10 membered ring having 0-4 heteroatoms; and t is 0, 1, 2, 3 or 4.

In some embodiments, L² is optionally substituted C₁₋₃ alkylene. In some embodiments, L² is C₁₋₃ alkylene. In some embodiments, L² is optionally substituted —CH₂—. In some embodiments, L² is optionally substituted —(CH₂)₂—. In some embodiments, L² is optionally substituted —(CH₂)₃—. In some embodiments, L² is —CH₂—. In some embodiments, L² is —(CH₂)₂—. In some embodiments, L² is —(CH₂)₃—.

In some embodiments, L² is optionally substituted C₁₋₃ alkylene, wherein one or more methylene unit of the alkylene is independently replaced with —O—. In some embodiments, L² is optionally substituted C₁₋₃ alkylene, wherein one unit of the alkylene is replaced with —O—. In some embodiments, L² is optionally substituted

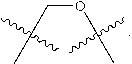 [SMILES: *CO*]

In some embodiments, L² is

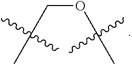 [SMILES: *CO*]

In some embodiments, L² is substituted

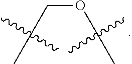 [SMILES: *CO*]

In some embodiments, L² is optionally substituted

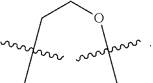 [SMILES: *CCO*]

In some embodiments, L² is

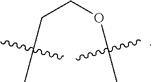 [SMILES: *CCO*]

In some embodiments, L² is substituted

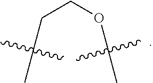 [SMILES: *CCO*]

In some embodiments, L² is an optionally substituted C₂₋₃ alkylene, wherein one unit of the alkylene is replaced with —O— and another unit of the alkylene is replaced with —C(R⁴)(R⁵)— and R⁴ and R⁵ are independently as described herein. In some embodiments, L² is

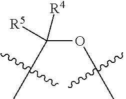

wherein R⁴ and R⁵ are independently as described herein. In some embodiments, L² is

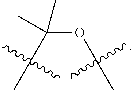 [SMILES: *OC(*)(C)C]

In some embodiments, L² is

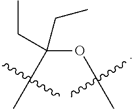 [SMILES: *OC(*)(CC)CC]

In some embodiments, L² is

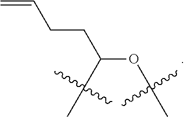 [SMILES: *OC(*)CCC=C]

In some embodiments, L² is

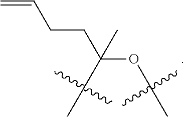 [SMILES: *OC(*)(C)CCC=C]

In some embodiments, L² is

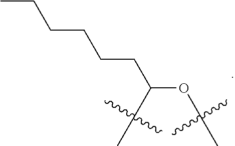 [SMILES: *OC(*)CCCCCC]

In some embodiments, L² is

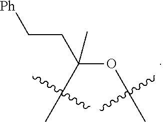 [SMILES: *OC(*)(C)CCc1ccccc1]

In some embodiments, L² is

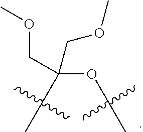 [SMILES: *OC(*)(COC)COC]

In some embodiments, L² is

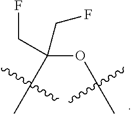 [SMILES: *OC(*)(CF)CF]

In some embodiments, L² is

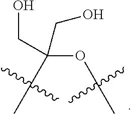 [SMILES: *OC(*)(CO)CO]

In some embodiments, L² is

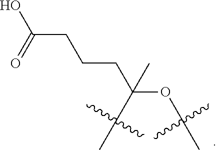 [SMILES: *OC(*)(C)CCCC(=O)O]

In some embodiments, L² is

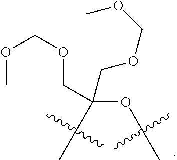 [SMILES: *OC(*)(COCOC)COCOC]

In some embodiments, L² is

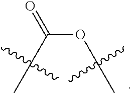 [SMILES: *OC(*)=O]

In some embodiments, R⁴ and R⁵ are taken together with the atom to which they are attached to form an optionally substituted ring as described herein.

In some embodiments, L² is optionally substituted C₁₋₃ alkylene, wherein one or more methylene unit of the alkylene is independently replaced with —S—. In some embodiments, L² is optionally substituted C₁₋₃ alkylene, wherein one methylene unit of the alkylene is replaced with —S—. In some embodiments, L² is optionally substituted

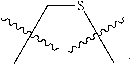 [SMILES: *CS*]

In some embodiments, L² is substituted

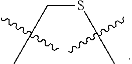 [SMILES: *CS*]

In some embodiments, L² is

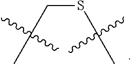 [SMILES: *CS*]

In some embodiments, L² is

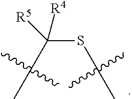

wherein R⁴ and R⁵ are independently as described herein. In some embodiments, each of R⁴ and R⁵ are independently optionally substituted C₁₋₆ aliphatic. For example, in some embodiments, each of R⁴ and R⁵ is methyl.

In some embodiments, L² is optionally substituted C₁₋₃ alkylene, wherein one or more methylene unit of the alkylene is independently replaced with —C(R⁴)(R⁵)— and R⁴ and R⁵ are independently as described herein. In some embodiments, R⁴ and R⁵ are taken together with the carbon atom to which they are attached to form an optionally substituted ring as described herein. In some embodiments, L² is optionally substituted C₁₋₃ alkylene, wherein one methylene unit of the alkylene is replaced with —C(R⁴)(R⁵)— and R⁴ and R⁵ are independently as described herein. In some embodiments, L² is

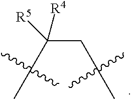

In some embodiments, L² is optionally substituted C₁₋₃ alkylene, wherein one or more methylene unit of the alkylene is independently replaced with —N(R⁴)— and R⁴ is as described herein. In some embodiments, L² is n optionally substituted C₁₋₃ alkylene, wherein one methylene unit of the alkylene is replaced with —N(R⁴)— and and R⁴ is as described herein. In some embodiments, L² is optionally substituted

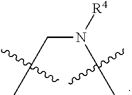

In some embodiments, L² is substituted

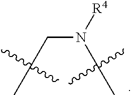

In some embodiments, L² is

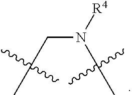

In some embodiments, L² is

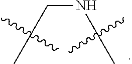 [SMILES: *CN*]

In some embodiments, L² is

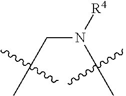

and R⁴ is C₁₋₆ alkyl. In some embodiments, L² is

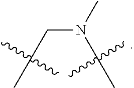 [SMILES: *CN(*)C]

In some embodiments, L² is

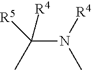

and each variable is independently as described herein. In some embodiments, L² is

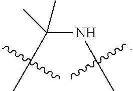 [SMILES: *NC(*)(C)C]

In some embodiments, L² is

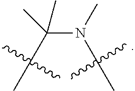 [SMILES: *N(C)C(*)(C)C]

In some embodiments, L² is

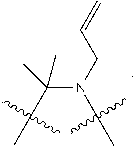 [SMILES: *N(CC=C)C(*)(C)C]

In some embodiments, L² is optionally substituted C₁₋₃ alkylene, wherein one or more methylene unit of the alkylene is independently replaced with optionally substituted —CH═N—. In some embodiments, L² is optionally substituted C₁₋₃ alkylene, wherein one methylene unit of the alkylene is replaced with optionally substituted —CH═N—. In some embodiments, L² is optionally substituted C₁₋₃ alkylene, wherein one methylene unit of the alkylene is replaced with —CH═N—. In some embodiments, L² is optionally substituted —CH═N—. In some embodiments, L² is —CH═N—. In some embodiments, L² is optionally substituted C₁₋₃ alkylene, wherein one or more methylene unit of the alkylene is independently replaced with optionally substituted —CH═CH—. In some embodiments, L² is optionally substituted C₁₋₃ alkylene, wherein one methylene unit of the alkylene is replaced with optionally substituted —CH═CH—. In some embodiments, L² is optionally substituted C₁₋₃ alkylene, wherein one methylene unit of the alkylene is replaced with —CH═CH—. In some embodiments, L² is optionally substituted —CH═CH—. In some embodiments, L² is —CH═CH—.

In some embodiments, L² is optionally substituted C₁₋₃ alkylene, wherein one or more methylene units of the alkylene are independently replaced with -Cy-, wherein -Cy- is as described herein. In some embodiments, L² is optionally substituted C₁₋₃ alkylene, wherein one methylene unit of the alkylene is replaced with -Cy-, wherein -Cy- is as described herein. In some embodiments, L² is —CH₂-Cy-, wherein the —CH₂— is optionally substituted and -Cy- is as described herein. In some embodiments, two methylene units are independently replaced wherein one of which with is replaced with -Cy-. In some embodiments, L² is -Cy-O— wherein -Cy- as described herein.

### -Cy-

As described herein, each -Cy- is independently

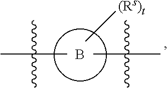

wherein Ring B is an optionally substituted (as those skilled in the art will appreciate, in addition to (R^{s}):) saturated or partially saturated 3-10 (e.g., 3, 4, 5, 6, 7, 8, 9, or 10) membered ring having 0-4 (e.g., 0, 1, 2, 3, or 4) heteroatoms.

### Ring B

Ring B can be various ring moieties as described herein. In some embodiments, Ring B is substituted in addition to (R^{s}) t. In some embodiments, Ring B is unsubstituted except (R^{s}) t. In some embodiments, Ring B is saturated. In some embodiments, Ring B is partially unsaturated. In some embodiments, Ring B is 3-7 membered. In some embodiments, Ring B is 3-membered. In some embodiments, Ring B is 4-membered. In some embodiments, Ring B is 5-membered. In some embodiments, Ring B is 6-membered. In some embodiments, Ring B is 7-membered. In some embodiments, Ring B is 8-membered. In some embodiments, Ring B is 9-membered. In some embodiments, Ring B is 10-membered. In some embodiments, Ring B is monocyclic. In some embodiments, Ring B is bicyclic. In some embodiments, Ring B is polycyclic. In some embodiments, each monocyclic unit is independently 3-7 membered, e.g., 3, 4, 5, 6, or 7-membered. In some embodiments, a ring has no heteroatoms. In some embodiments, there is one heteroatom in a ring. In some embodiments, there are two heteroatoms in a ring. In some embodiments, there are three heteroatoms in a ring. In some embodiments, there are four heteroatoms in a ring. In some embodiments, Ring B is an optionally substituted saturated 3-10 (e.g., 3, 4, 5, 6, 7, 8, 9, or 10) membered ring having 0-4 (e.g., 0, 1, 2, 3, or 4) heteroatoms. In some embodiments, Ring B is an optionally substituted partially unsaturated 3-10 (e.g., 3, 4, 5, 6, 7, 8, 9, or 10) membered ring having 0-4 (e.g., 0, 1, 2, 3, or 4) heteroatoms. In some embodiments, Ring B is an optionally substituted saturated 3-7 membered ring having 0-4 heteroatoms. In some embodiments, Ring B is an optionally substituted saturated 3-membered ring having no heteroatoms. In some embodiments, Ring B is an optionally substituted saturated 3 membered ring having 1 heteroatom. In some embodiments, Ring B is an optionally substituted saturated 4-membered ring having no heteroatoms. In some embodiments, Ring B is an optionally substituted saturated 4-membered ring having 1 heteroatom. In some embodiments, Ring B is an optionally substituted saturated 5-membered ring having no heteroatoms. In some embodiments, Ring B is an optionally substituted saturated 5-membered ring having 1 heteroatom. In some embodiments, Ring B is an optionally substituted saturated 5-membered ring having 2 heteroatoms. In some embodiments, Ring B is an optionally substituted saturated 6-membered ring having no heteroatoms. In some embodiments, Ring B is an optionally substituted saturated 6-membered ring having 1 heteroatom. In some embodiments, Ring B is an optionally substituted saturated 6-membered ring having 2 heteroatoms and each variable is independently as described herein. In some embodiments, Ring B is an optionally substituted saturated 7-membered ring having no heteroatoms. In some embodiments, Ring B is an optionally substituted saturated 7-membered ring having 1 heteroatom. In some embodiments, Ring B is an optionally substituted saturated 7-membered ring having 2 heteroatoms and each variable is independently as described herein.

In some embodiments, a heteroatom is nitrogen. In some embodiments, a nitrogen atom is substituted. In some embodiments, a nitrogen atom is bonded to R^{s} which is as described herein. In some embodiments, a heteroatom is oxygen. In some embodiments, a heteroatom is sulfur. In some embodiments, a sulfur atom is substituted (e.g., —S(O)₂—).

In some embodiments, Ring B is optionally substituted

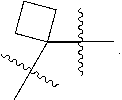 [SMILES: *C1(*)CCC1]

In some embodiments, Ring B is optionally substituted

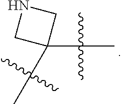 [SMILES: *C1(*)CNC1]

In some embodiments, Ring B is optionally substituted

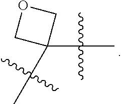 [SMILES: *C1(*)COC1]

In some embodiments, Ring B is optionally substituted

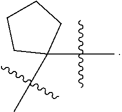 [SMILES: *C1(*)CCCC1]

In some embodiments, Ring B is optionally substituted

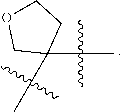 [SMILES: *C1(*)CCOC1]

In some embodiments, Ring B is optionally substituted

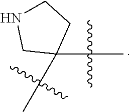 [SMILES: *C1(*)CCNC1]

In some embodiments, Ring B is optionally substituted

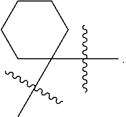 [SMILES: *C1(*)CCCCC1]

In some embodiments, Ring B is optionally substituted

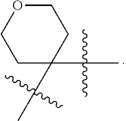 [SMILES: *C1(*)CCOCC1]

In some embodiments, Ring B is optionally substituted

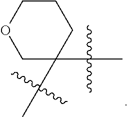 [SMILES: *C1(*)CCCOC1]

In some embodiments, Ring B is optionally substituted

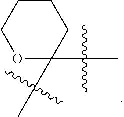 [SMILES: *C1(*)CCCCO1]

In some embodiments, Ring B is optionally substituted

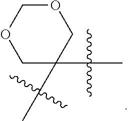 [SMILES: *C1(*)COCOC1]

In some embodiments, Ring B is optionally substituted

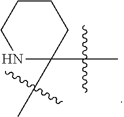 [SMILES: *C1(*)CCCCN1]

In some embodiments, Ring B is optionally substituted

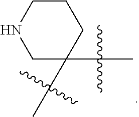 [SMILES: *C1(*)CCCNC1]

In some embodiments, Ring B is optionally substituted

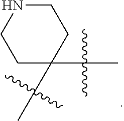 [SMILES: *C1(*)CCNCC1]

In some embodiments, Ring B is optionally substituted

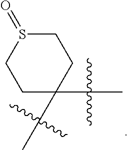 [SMILES: *C1(*)CCS(=O)CC1]

In some embodiments, Ring B is optionally substituted

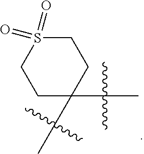 [SMILES: *C1(*)CCS(=O)(=O)CC1]

In some embodiments, Ring B is optionally substituted

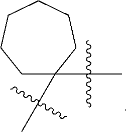 [SMILES: *C1(*)CCCCCC1]

In some embodiments, Ring B is optionally substituted

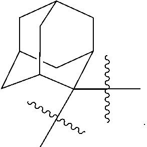 [SMILES: *C1(*)C2CC3CC(C2)CC1C3]

t

In some embodiments, t is 0. In some embodiments, t is 1. In some embodiments, t is 2. In some embodiments, t is 3. In some embodiments, t is 4.

In some embodiments, -Cy- is bonded to the rest of a molecule at a single atom. In some embodiments, -Cy- is spirocyclic. In some embodiments, -Cy- is

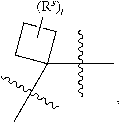

wherein each of R^{s} and t is independently as described herein. In some embodiments, -Cy- is

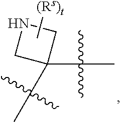

wherein each of R^{s} and t is independently as described herein. In some embodiments, -Cy- is

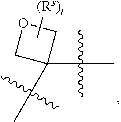

wherein each of R^{s} and t is independently as described herein.

In some embodiments, -Cy- is

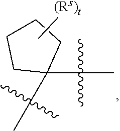

wherein each of R^{s} and t is independently as described herein. In some embodiments, -Cy- is

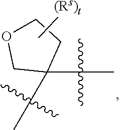

wherein each of R^{s} and t is independently as described herein. In some embodiments, -Cy- is

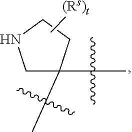

wherein each of R^{s} and t is independently as described herein.

In some embodiments, -Cy- is

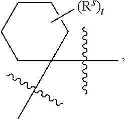

wherein each of R^{s} and t is independently as described herein. In some embodiments, Cy- is

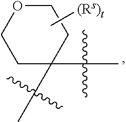

wherein each of R^{s} and t is independently as described herein. In some embodiments, Cy- is

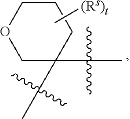

wherein each of R^{s} and t is independently as described herein. In some embodiments, -Cy- is

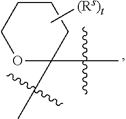

wherein each of R^{s} and t is independently as described herein. In some embodiments, -Cy- is

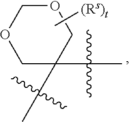

wherein each of R^{s} and t is independently as described herein. In some embodiments, -Cy- is

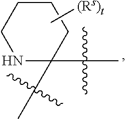

wherein each of R^{s} and t is independently as described herein. In some embodiments, -Cy- is

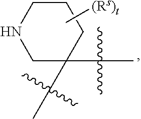

wherein each of R^{s} and t is independently as described herein. In some embodiments, -Cy- is

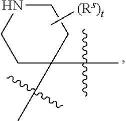

wherein each of R^{s} and t is independently as described herein. In some embodiments, -Cy- is

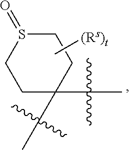

wherein each of R^{s} and t is independently as described herein. In some embodiments, -Cy- is

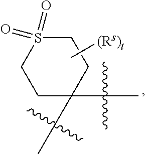

wherein each of R^{s} and t is independently as described herein. In some embodiments, -Cy- is

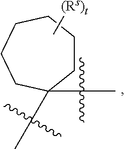

wherein each of R^{s} and t is independently as described herein. In some embodiments, Cy- is

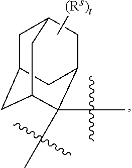

wherein each of R^{s} and t is independently as described herein.

In some embodiments, the present disclosure provides a compound having a structure of

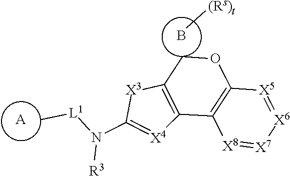

or a salt thereof.

In some embodiments, L² is

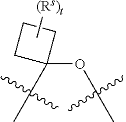

wherein each variable is as described herein. In some embodiments, L² is

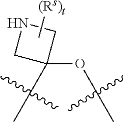

wherein each variable is as described herein. In some embodiments, L² is

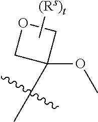

wherein each variable is as described herein. In some embodiments, L² is

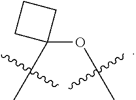 [SMILES: *OC1(*)CCC1]

In some embodiments, L² is

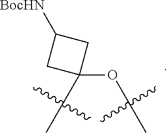 [SMILES: *OC1(*)CC(NC(=O)OC(C)(C)C)C1]

In some embodiments, L² is

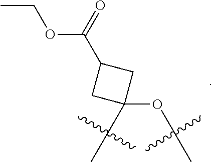 [SMILES: *OC1(*)CC(C(=O)OCC)C1]

In some embodiments, L² is

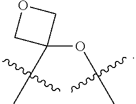 [SMILES: *OC1(*)COC1]

In some embodiments, L² is

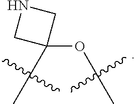 [SMILES: *OC1(*)CNC1]

In some embodiments, L² is

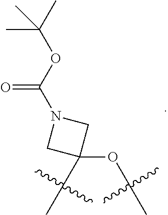 [SMILES: *OC1(*)CN(C(=O)OC(C)(C)C)C1]

In some embodiments. L² is

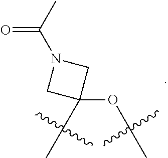 [SMILES: *OC1(*)CN(C(C)=O)C1]

In some embodiments, L² is

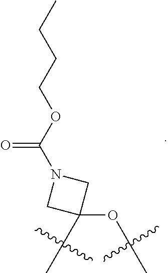 [SMILES: *OC1(*)CN(C(=O)OCCCC)C1]

In some embodiments, L² is

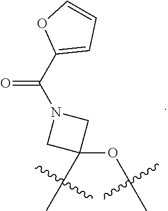 [SMILES: *OC1(*)CN(C(=O)c2ccco2)C1]

In some embodiments, L² is

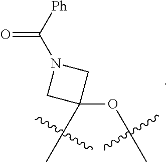 [SMILES: *OC1(*)CN(C(=O)c2ccccc2)C1]

In some embodiments, L² is

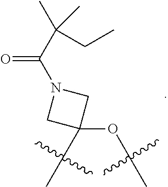 [SMILES: *OC1(*)CN(C(=O)C(C)(C)CC)C1]

In some embodiments, L² is

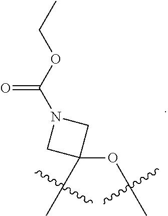 [SMILES: *OC1(*)CN(C(=O)OCC)C1]

In some embodiments, L² is

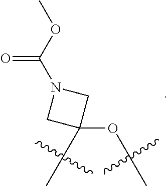 [SMILES: *OC1(*)CN(C(=O)OC)C1]

In some embodiments, L² is

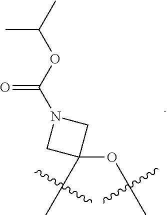 [SMILES: *OC1(*)CN(C(=O)OC(C)C)C1]

In some embodiments, L² is

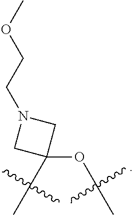 [SMILES: *OC1(*)CN(CCOC)C1]

In some embodiments, L² is

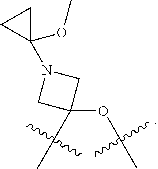 [SMILES: *OC1(*)CN(C2(OC)CC2)C1]

In some embodiments, L² is

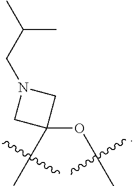 [SMILES: *OC1(*)CN(CC(C)C)C1]

In some embodiments, L² is

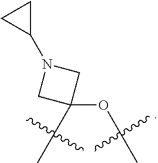 [SMILES: *OC1(*)CN(C2CC2)C1]

In some embodiments, L² is

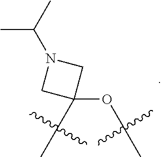 [SMILES: *OC1(*)CN(C(C)C)C1]

In some embodiments, L² is

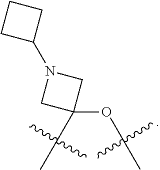 [SMILES: *OC1(*)CN(C2CCC2)C1]

In some embodiments, L² is

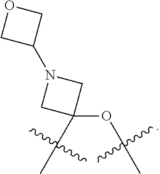 [SMILES: *OC1(*)CN(C2COC2)C1]

In some embodiments, L² is

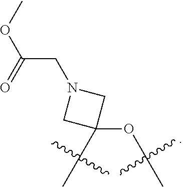 [SMILES: *OC1(*)CN(CC(=O)OC)C1]

In some embodiments, L² is

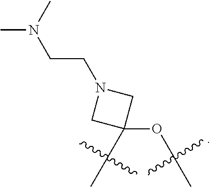 [SMILES: *OC1(*)CN(CCN(C)C)C1]

In some embodiments, L² is

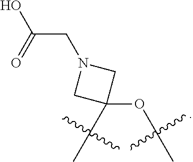 [SMILES: *OC1(*)CN(CC(=O)O)C1]

In some embodiments, L² is

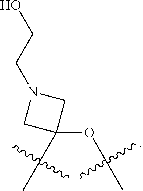 [SMILES: *OC1(*)CN(CCO)C1]

In some embodiments, L² is

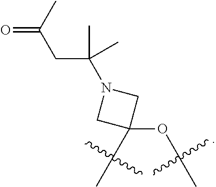 [SMILES: *OC1(*)CN(C(C)(C)CC(C)=O)C1]

In some embodiments, L² is

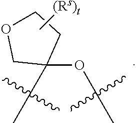

wherein each variable is as described herein. In some embodiments, L² is

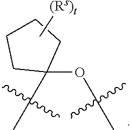

wherein each variable is as described herein. In some embodiments, L² is

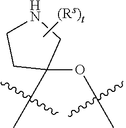

wherein each variable is as described herein. In some embodiments, L² is

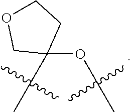 [SMILES: *OC1(*)CCOC1]

In some embodiments, L² is

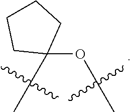 [SMILES: *OC1(*)CCCC1]

In some embodiments, L² is

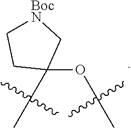 [SMILES: *OC1(*)CCN(C(=O)OC(C)(C)C)C1]

In some embodiments, L² is

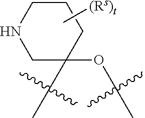

wherein each variable is as described herein. In some embodiments, L² is

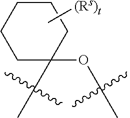

wherein each variable is as described herein. In some embodiments, L² is

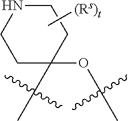

wherein each variable is as described herein. In some embodiments. L² is

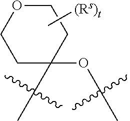

wherein each variable is as described herein. In some embodiments, L² is

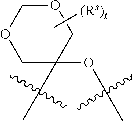

wherein each variable is as described herein. In some embodiments, L² is

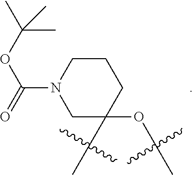 [SMILES: *OC1(*)CCCN(C(=O)OC(C)(C)C)C1]

In some embodiments, L² is

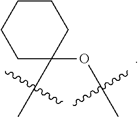 [SMILES: *OC1(*)CCCCC1]

In some embodiments, L² is

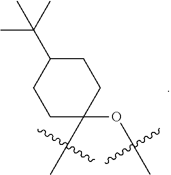 [SMILES: *OC1(*)CCC(C(C)(C)C)CC1]

In some embodiments, L² is

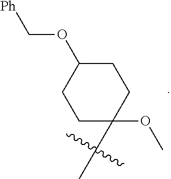 [SMILES: *C1(OC)CCC(OCc2ccccc2)CC1]

In some embodiments, L² is

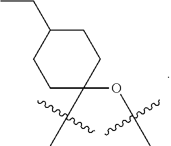 [SMILES: *OC1(*)CCC(CC)CC1]

In some embodiments, L² is

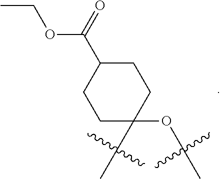 [SMILES: *OC1(*)CCC(C(=O)OCC)CC1]

In some embodiments, L² is

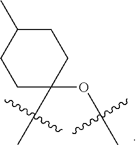 [SMILES: *OC1(*)CCC(C)CC1]

In some embodiments, L² is

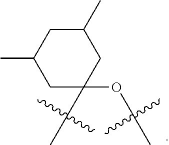 [SMILES: *OC1(*)CC(C)CC(C)C1]

In some embodiments, L² is

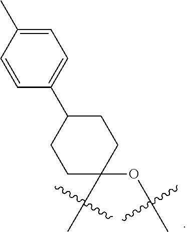 [SMILES: *OC1(*)CCC(c2ccc(C)cc2)CC1]

In some embodiments, L² is

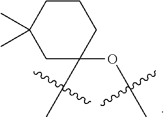 [SMILES: *OC1(*)CCCC(C)(C)C1]

In some embodiments, L² is

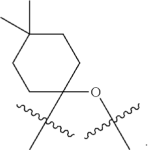 [SMILES: *OC1(*)CCC(C)(C)CC1]

In some embodiments, L² is

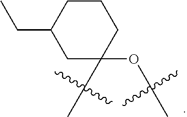 [SMILES: *OC1(*)CCCC(CC)C1]

In some embodiments, L² is

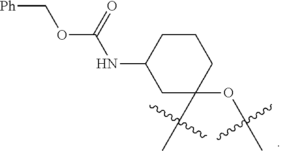 [SMILES: *OC1(*)CCCC(NC(=O)OCc2ccccc2)C1]

In some embodiments, L² is

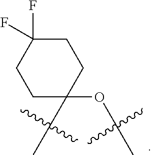 [SMILES: *OC1(*)CCC(F)(F)CC1]

In some embodiments, L² is

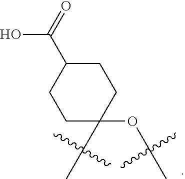 [SMILES: *OC1(*)CCC(C(=O)O)CC1]

In some embodiments, L² is

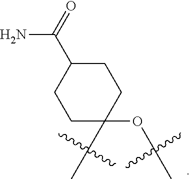 [SMILES: *OC1(*)CCC(C(N)=O)CC1]

In some embodiments, L² is

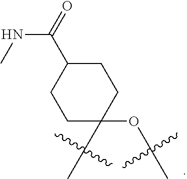 [SMILES: *OC1(*)CCC(C(=O)NC)CC1]

In some embodiments, L² is

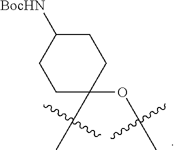 [SMILES: *OC1(*)CCC(NC(=O)OC(C)(C)C)CC1]

In some embodiments, L² is

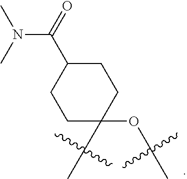 [SMILES: *OC1(*)CCC(C(=O)N(C)C)CC1]

In some embodiments, L² is

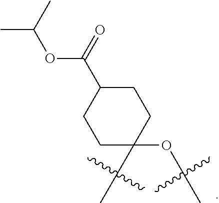 [SMILES: *OC1(*)CCC(C(=O)OC(C)C)CC1]

In some embodiments, L² is

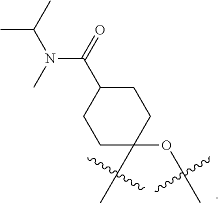 [SMILES: *OC1(*)CCC(C(=O)N(C)C(C)C)CC1]

In some embodiments, L² is

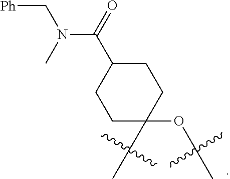 [SMILES: *OC1(*)CCC(C(=O)N(C)Cc2ccccc2)CC1]

In some embodiments, L² is

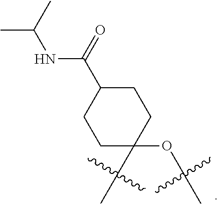 [SMILES: *OC1(*)CCC(C(=O)NC(C)C)CC1]

In some embodiments, L² is

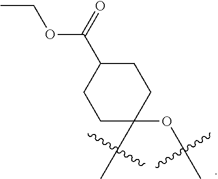 [SMILES: *OC1(*)CCC(C(=O)OCC)CC1]

In some embodiments, L² is

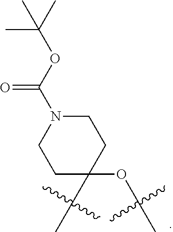 [SMILES: *OC1(*)CCN(C(=O)OC(C)(C)C)CC1]

In some embodiments, L² is

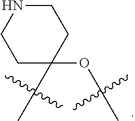 [SMILES: *OC1(*)CCNCC1]

In some embodiments, L² is

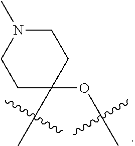 [SMILES: *OC1(*)CCN(C)CC1]

In some embodiments, L² is

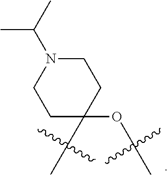 [SMILES: *OC1(*)CCN(C(C)C)CC1]

In some embodiments, L² is

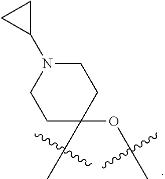 [SMILES: *OC1(*)CCN(C2CC2)CC1]

In some embodiments, L² is

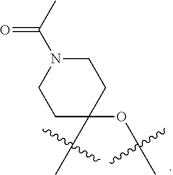 [SMILES: *OC1(*)CCN(C(C)=O)CC1]

In some embodiments, L² is

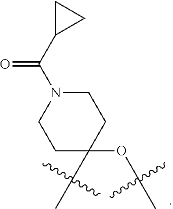 [SMILES: *OC1(*)CCN(C(=O)C2CC2)CC1]

In some embodiments, L² is

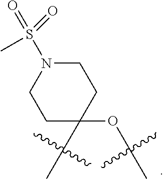 [SMILES: *OC1(*)CCN(S(C)(=O)=O)CC1]

In some embodiments, L² is

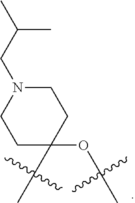 [SMILES: *OC1(*)CCN(CC(C)C)CC1]

In some embodiments, L² is

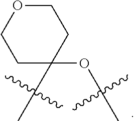 [SMILES: *OC1(*)CCOCC1]

In some embodiments, L² is

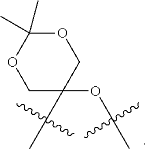 [SMILES: *OC1(*)COC(C)(C)OC1]

In some embodiments, L² is

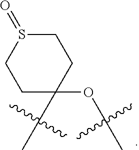 [SMILES: *OC1(*)CCS(=O)CC1]

In some embodiments, L² is

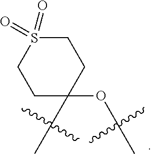 [SMILES: *OC1(*)CCS(=O)(=O)CC1]

In some embodiments, L² is

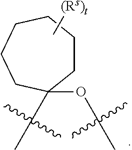

wherein each variable is as described herein. In some embodiments, L² is

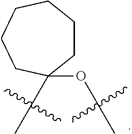 [SMILES: *OC1(*)CCCCCC1]

In some embodiments, L² is

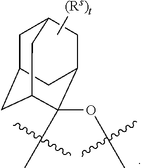

wherein each variable is as described herein.

In some embodiments, the present disclosure provides a compound having a structure of

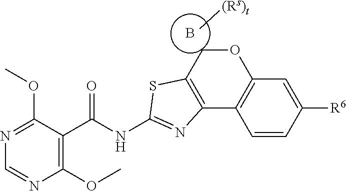

or a salt thereof, wherein each variable is independently as described herein.

In some embodiments, the present disclosure provides a compound having a structure of

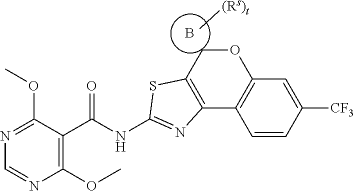

or a salt thereof, wherein each variable is independently as described herein.

In some embodiments, the present disclosure provides a compound having a structure of

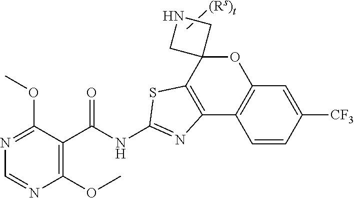

or a salt thereof, wherein each variable is independently as described herein.

In some embodiments, the present disclosure provides a compound having a structure of

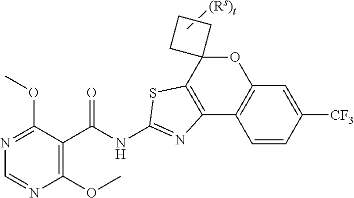

or a salt thereof, wherein each variable is independently as described herein.

In some embodiments, the present disclosure provides a compound having a structure of

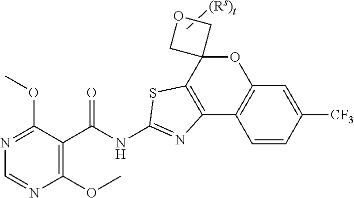

or a salt thereof, wherein each variable is independently as described herein.

In some embodiments, the present disclosure provides a compound having a structure of

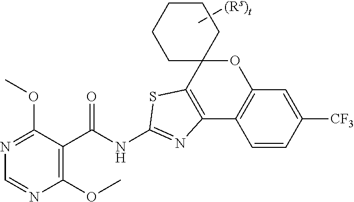

or a salt thereof, wherein each variable is independently as described herein.

In some embodiments, the present disclosure provides a compound having a structure of

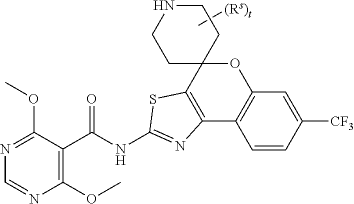

or a salt thereof, wherein each variable is independently as described herein.

In some embodiments, the present disclosure provides a compound having a structure selected below or a salt thereof. In some embodiments, a salt is a pharmaceutically acceptable salt. In some embodiments, a compound may optionally in a solvate form.

| Compound | Structure |
|---|---|
| 1 | 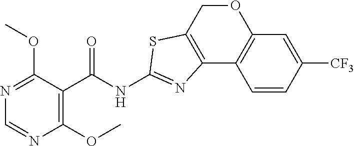 [SMILES: COc1ncnc(OC)c1C(=O)Nc1nc2c(s1)COc1cc(C(F)(F)F)ccc1-2] |
| 2 | 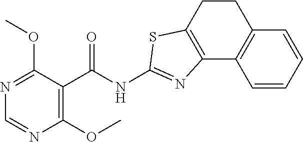 [SMILES: COc1ncnc(OC)c1C(=O)Nc1nc2c(s1)CCc1ccccc1-2] |
| 3 | 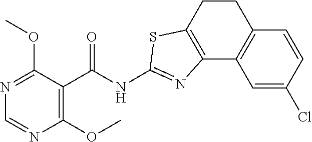 [SMILES: COc1ncnc(OC)c1C(=O)Nc1nc2c(s1)CCc1ccc(Cl)cc1-2] |
| 4 | 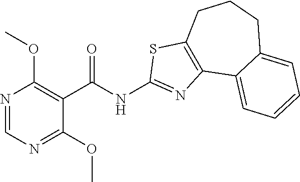 [SMILES: COc1ncnc(OC)c1C(=O)Nc1nc2c(s1)CCCc1ccccc1-2] |
| 5 | 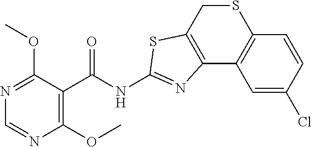 [SMILES: COc1ncnc(OC)c1C(=O)Nc1nc2c(s1)CSc1ccc(Cl)cc1-2] |
| 6 | 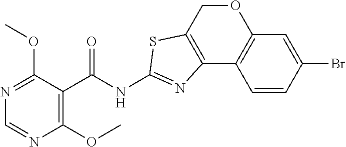 [SMILES: COc1ncnc(OC)c1C(=O)Nc1nc2c(s1)COc1cc(Br)ccc1-2] |
| 7 | 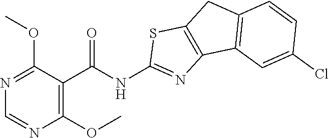 [SMILES: COc1ncnc(OC)c1C(=O)Nc1nc2c(s1)Cc1ccc(Cl)cc1-2] |
| 8 | 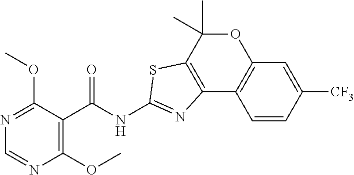 [SMILES: COc1ncnc(OC)c1C(=O)Nc1nc2c(s1)C(C)(C)Oc1cc(C(F)(F)F)ccc1-2] |
| 9 | 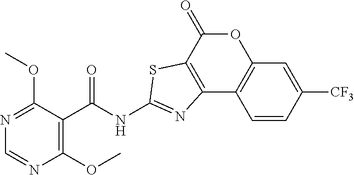 [SMILES: COc1ncnc(OC)c1C(=O)Nc1nc2c(s1)c(=O)oc1cc(C(F)(F)F)ccc12] |
| 10 | 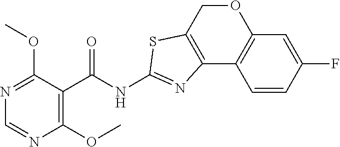 [SMILES: COc1ncnc(OC)c1C(=O)Nc1nc2c(s1)COc1cc(F)ccc1-2] |
| 11 | 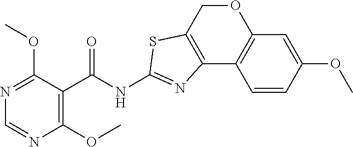 |
| 12 | 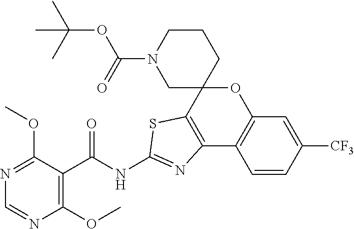 [SMILES: COc1ncnc(OC)c1C(=O)Nc1nc2c(s1)C1(CCCN(C(=O)OC(C)(C)C)C1)Oc1cc(C(F)(F)F)ccc1-2] |
| 13 | 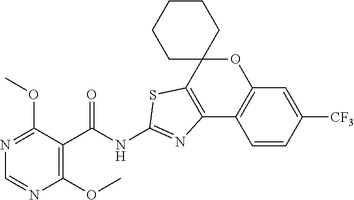 [SMILES: COc1ncnc(OC)c1C(=O)Nc1nc2c(s1)C1(CCCCC1)Oc1cc(C(F)(F)F)ccc1-2] |
| 14 | 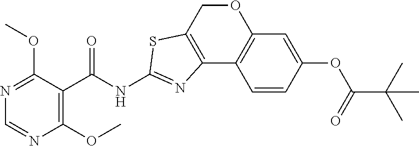 [SMILES: COc1ncnc(OC)c1C(=O)Nc1nc2c(s1)COc1cc(OC(=O)C(C)(C)C)ccc1-2] |
| 15 | 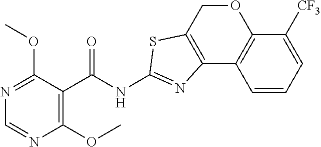 [SMILES: COc1ncnc(OC)c1C(=O)Nc1nc2c(s1)COc1c-2cccc1C(F)(F)F] |
| 16 | 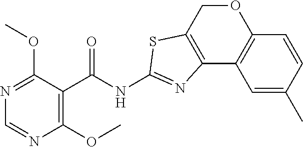 |
| 17 | 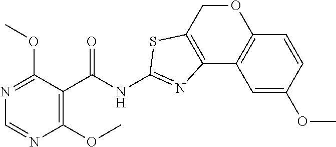 |
| 18 | 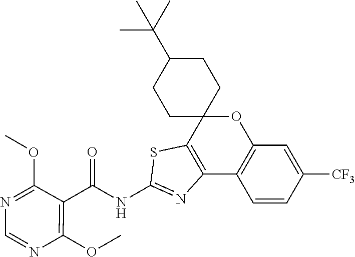 [SMILES: COc1ncnc(OC)c1C(=O)Nc1nc2c(s1)C1(CCC(C(C)(C)C)CC1)Oc1cc(C(F)(F)F)ccc1-2] |
| 19 | 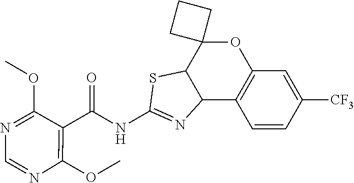 [SMILES: COc1ncnc(OC)c1C(=O)NC1=NC2c3ccc(C(F)(F)F)cc3OC3(CCC3)C2S1] |
| 20 | 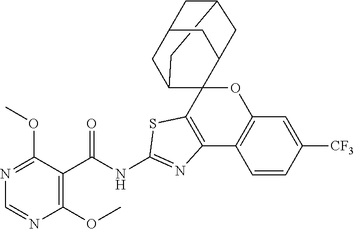 [SMILES: COc1ncnc(OC)c1C(=O)Nc1nc2c(s1)C1(Oc3cc(C(F)(F)F)ccc3-2)C2CC3CC(C2)CC1C3] |
| 21 | 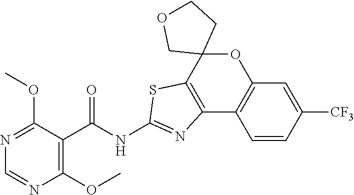 [SMILES: COc1ncnc(OC)c1C(=O)Nc1nc2c(s1)C1(CCOC1)Oc1cc(C(F)(F)F)ccc1-2] |
| 22 | 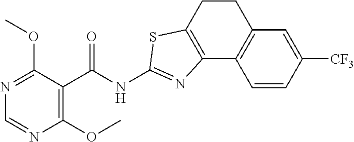 [SMILES: COc1ncnc(OC)c1C(=O)Nc1nc2c(s1)CCc1cc(C(F)(F)F)ccc1-2] |
| 23 | 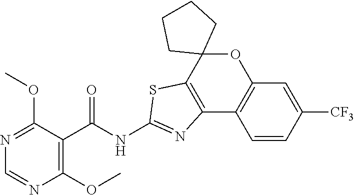 [SMILES: COc1ncnc(OC)c1C(=O)Nc1nc2c(s1)C1(CCCC1)Oc1cc(C(F)(F)F)ccc1-2] |
| 24 | 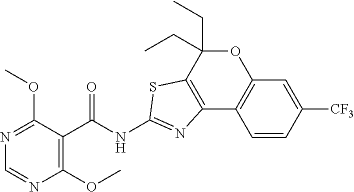 [SMILES: CCC1(CC)Oc2cc(C(F)(F)F)ccc2-c2nc(NC(=O)c3c(OC)ncnc3OC)sc21] |
| 25 | 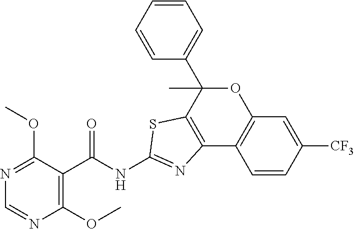 [SMILES: COc1ncnc(OC)c1C(=O)Nc1nc2c(s1)C(C)(c1ccccc1)Oc1cc(C(F)(F)F)ccc1-2] |
| 26 | 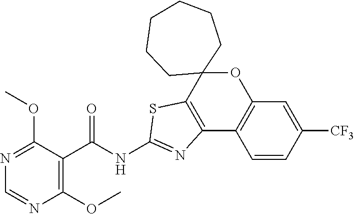 [SMILES: COc1ncnc(OC)c1C(=O)Nc1nc2c(s1)C1(CCCCCC1)Oc1cc(C(F)(F)F)ccc1-2] |
| 27 | 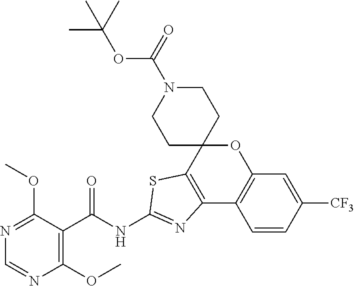 [SMILES: COc1ncnc(OC)c1C(=O)Nc1nc2c(s1)C1(CCN(C(=O)OC(C)(C)C)CC1)Oc1cc(C(F)(F)F)ccc1-2] |
| 28 | 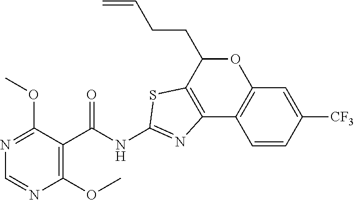 [SMILES: C=CCCC1Oc2cc(C(F)(F)F)ccc2-c2nc(NC(=O)c3c(OC)ncnc3OC)sc21] |
| 29 | 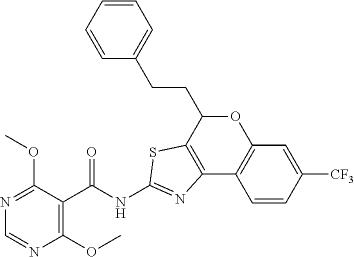 [SMILES: COc1ncnc(OC)c1C(=O)Nc1nc2c(s1)C(CCc1ccccc1)Oc1cc(C(F)(F)F)ccc1-2] |
| 30 | 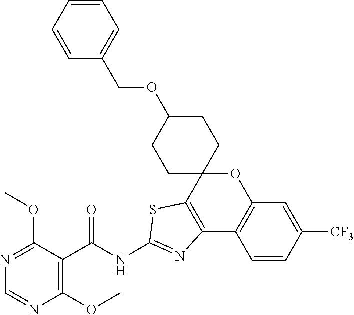 [SMILES: COc1ncnc(OC)c1C(=O)Nc1nc2c(s1)C1(CCC(OCc3ccccc3)CC1)Oc1cc(C(F)(F)F)ccc1-2] |
| 31 | 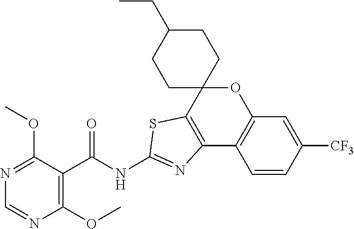 [SMILES: CCC1CCC2(CC1)Oc1cc(C(F)(F)F)ccc1-c1nc(NC(=O)c3c(OC)ncnc3OC)sc12] |
| 32 | 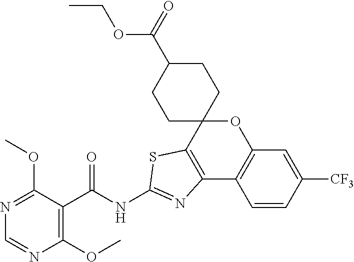 [SMILES: CCOC(=O)C1CCC2(CC1)Oc1cc(C(F)(F)F)ccc1-c1nc(NC(=O)c3c(OC)ncnc3OC)sc12] |
| 33 | 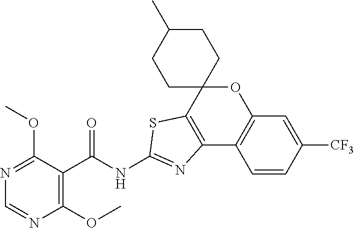 [SMILES: COc1ncnc(OC)c1C(=O)Nc1nc2c(s1)C1(CCC(C)CC1)Oc1cc(C(F)(F)F)ccc1-2] |
| 34 | 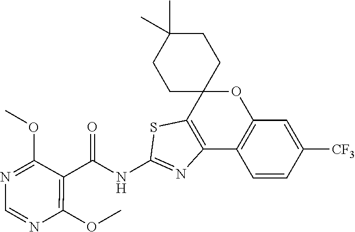 [SMILES: COc1ncnc(OC)c1C(=O)Nc1nc2c(s1)C1(CCC(C)(C)CC1)Oc1cc(C(F)(F)F)ccc1-2] |
| 35 | 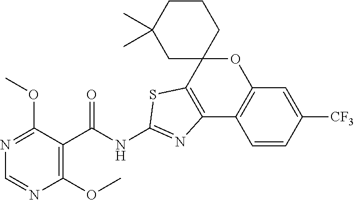 [SMILES: COc1ncnc(OC)c1C(=O)Nc1nc2c(s1)C1(CCCC(C)(C)C1)Oc1cc(C(F)(F)F)ccc1-2] |
| 36 | 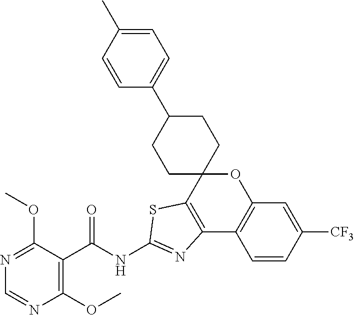 [SMILES: COc1ncnc(OC)c1C(=O)Nc1nc2c(s1)C1(CCC(c3ccc(C)cc3)CC1)Oc1cc(C(F)(F)F)ccc1-2] |
| 37 | 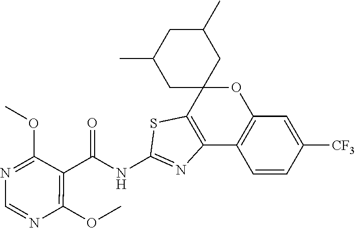 [SMILES: COc1ncnc(OC)c1C(=O)Nc1nc2c(s1)C1(CC(C)CC(C)C1)Oc1cc(C(F)(F)F)ccc1-2] |
| 38 | 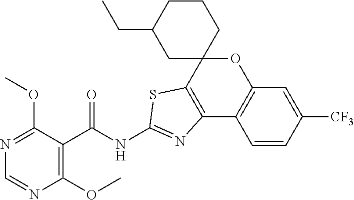 [SMILES: CCC1CCCC2(C1)Oc1cc(C(F)(F)F)ccc1-c1nc(NC(=O)c3c(OC)ncnc3OC)sc12] |
| 39 | 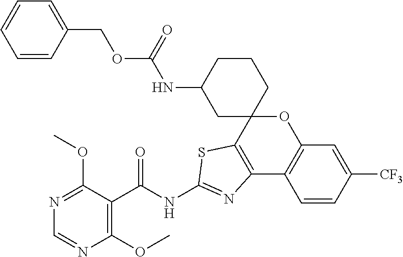 [SMILES: COc1ncnc(OC)c1C(=O)Nc1nc2c(s1)C1(CCCC(NC(=O)OCc3ccccc3)C1)Oc1cc(C(F)(F)F)ccc1-2] |
| 40 | 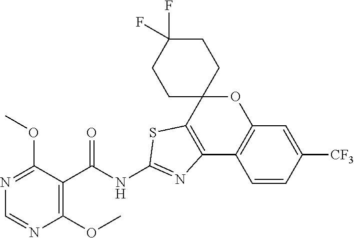 [SMILES: COc1ncnc(OC)c1C(=O)Nc1nc2c(s1)C1(CCC(F)(F)CC1)Oc1cc(C(F)(F)F)ccc1-2] |
| 41 | 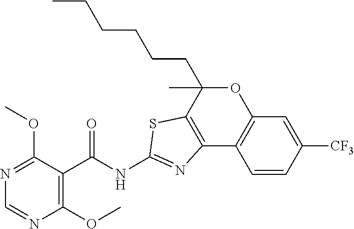 [SMILES: CCCCCCC1(C)Oc2cc(C(F)(F)F)ccc2-c2nc(NC(=O)c3c(OC)ncnc3OC)sc21] |
| 42 | 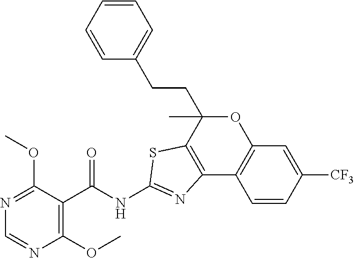 [SMILES: COc1ncnc(OC)c1C(=O)Nc1nc2c(s1)C(C)(CCc1ccccc1)Oc1cc(C(F)(F)F)ccc1-2] |
| 43 | 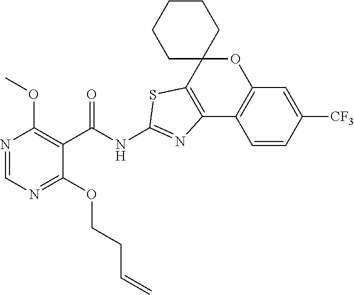 [SMILES: C=CCCOc1ncnc(OC)c1C(=O)Nc1nc2c(s1)C1(CCCCC1)Oc1cc(C(F)(F)F)ccc1-2] |
| 44 | 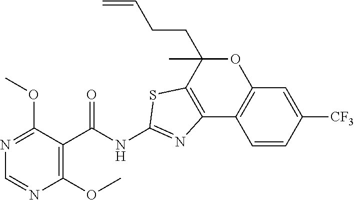 [SMILES: C=CCCC1(C)Oc2cc(C(F)(F)F)ccc2-c2nc(NC(=O)c3c(OC)ncnc3OC)sc21] |
| 45 | 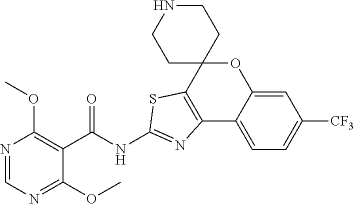 [SMILES: COc1ncnc(OC)c1C(=O)Nc1nc2c(s1)C1(CCNCC1)Oc1cc(C(F)(F)F)ccc1-2] |
| 46 | 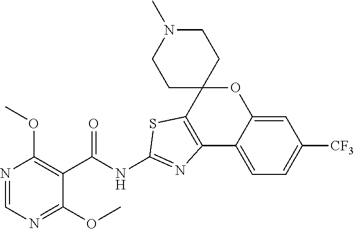 [SMILES: COc1ncnc(OC)c1C(=O)Nc1nc2c(s1)C1(CCN(C)CC1)Oc1cc(C(F)(F)F)ccc1-2] |
| 47 | 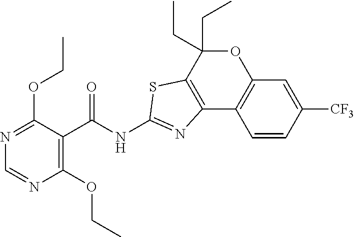 [SMILES: CCOc1ncnc(OCC)c1C(=O)Nc1nc2c(s1)C(CC)(CC)Oc1cc(C(F)(F)F)ccc1-2] |
| 48 | 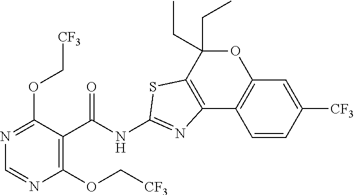 [SMILES: CCC1(CC)Oc2cc(C(F)(F)F)ccc2-c2nc(NC(=O)c3c(OCC(F)(F)F)ncnc3OCC(F)(F)F)sc21] |
| 49 | 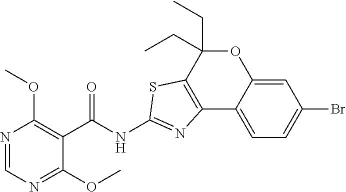 [SMILES: CCC1(CC)Oc2cc(Br)ccc2-c2nc(NC(=O)c3c(OC)ncnc3OC)sc21] |
| 50 | 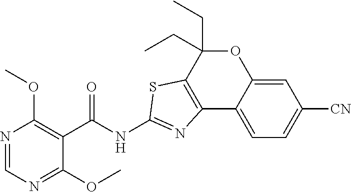 [SMILES: CCC1(CC)Oc2cc(C#N)ccc2-c2nc(NC(=O)c3c(OC)ncnc3OC)sc21] |
| 51 | 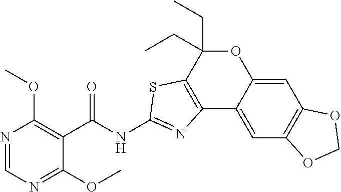 [SMILES: CCC1(CC)Oc2cc3c(cc2-c2nc(NC(=O)c4c(OC)ncnc4OC)sc21)OCO3] |
| 52 | 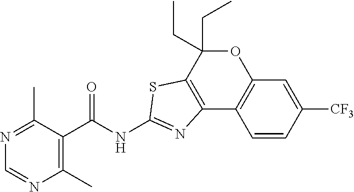 [SMILES: CCC1(CC)Oc2cc(C(F)(F)F)ccc2-c2nc(NC(=O)c3c(C)ncnc3C)sc21] |
| 53 | 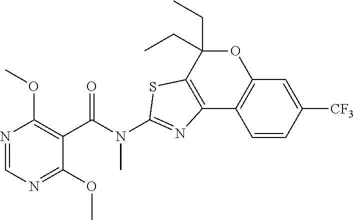 [SMILES: CCC1(CC)Oc2cc(C(F)(F)F)ccc2-c2nc(N(C)C(=O)c3c(OC)ncnc3OC)sc21] |
| 54 | 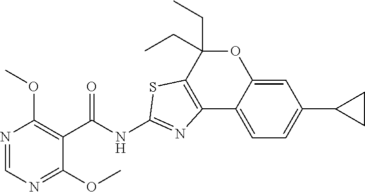 [SMILES: CCC1(CC)Oc2cc(C3CC3)ccc2-c2nc(NC(=O)c3c(OC)ncnc3OC)sc21] |
| 55 | 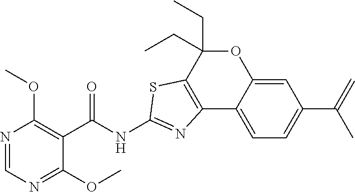 [SMILES: C=C(C)c1ccc2c(c1)OC(CC)(CC)c1sc(NC(=O)c3c(OC)ncnc3OC)nc1-2] |
| 56 | 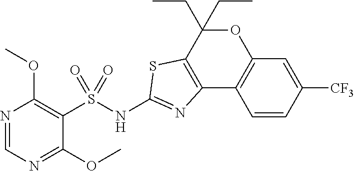 [SMILES: CCC1(CC)Oc2cc(C(F)(F)F)ccc2-c2nc(NS(=O)(=O)c3c(OC)ncnc3OC)sc21] |
| 57 | 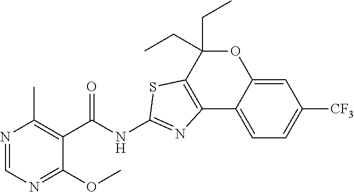 [SMILES: CCC1(CC)Oc2cc(C(F)(F)F)ccc2-c2nc(NC(=O)c3c(C)ncnc3OC)sc21] |
| 58 | 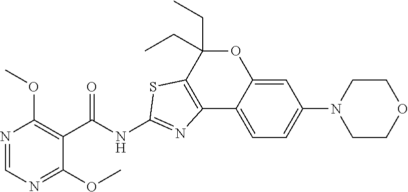 [SMILES: CCC1(CC)Oc2cc(N3CCOCC3)ccc2-c2nc(NC(=O)c3c(OC)ncnc3OC)sc21] |
| 59 | 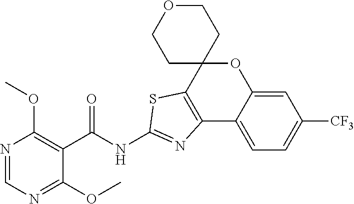 [SMILES: COc1ncnc(OC)c1C(=O)Nc1nc2c(s1)C1(CCOCC1)Oc1cc(C(F)(F)F)ccc1-2] |
| 60 | 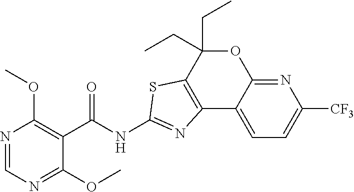 [SMILES: CCC1(CC)Oc2nc(C(F)(F)F)ccc2-c2nc(NC(=O)c3c(OC)ncnc3OC)sc21] |
| 61 | 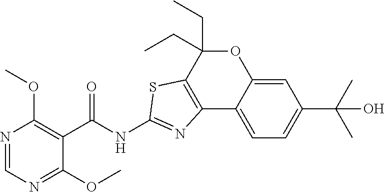 [SMILES: CCC1(CC)Oc2cc(C(C)(C)O)ccc2-c2nc(NC(=O)c3c(OC)ncnc3OC)sc21] |
| 62 | 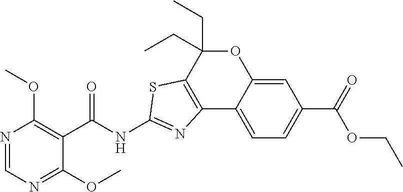 [SMILES: CCOC(=O)c1ccc2c(c1)OC(CC)(CC)c1sc(NC(=O)c3c(OC)ncnc3OC)nc1-2] |
| 63 | 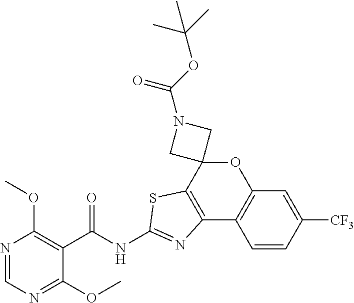 [SMILES: COc1ncnc(OC)c1C(=O)Nc1nc2c(s1)C1(CN(C(=O)OC(C)(C)C)C1)Oc1cc(C(F)(F)F)ccc1-2] |
| 64 | 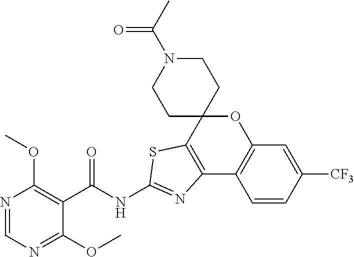 [SMILES: COc1ncnc(OC)c1C(=O)Nc1nc2c(s1)C1(CCN(C(C)=O)CC1)Oc1cc(C(F)(F)F)ccc1-2] |
| 65 | 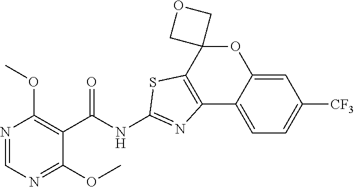 [SMILES: COc1ncnc(OC)c1C(=O)Nc1nc2c(s1)C1(COC1)Oc1cc(C(F)(F)F)ccc1-2] |
| 66 | 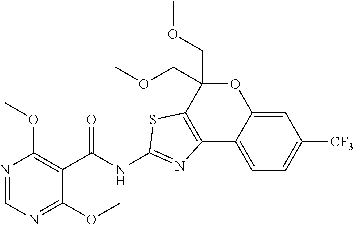 [SMILES: COCC1(COC)Oc2cc(C(F)(F)F)ccc2-c2nc(NC(=O)c3c(OC)ncnc3OC)sc21] |
| 67 | 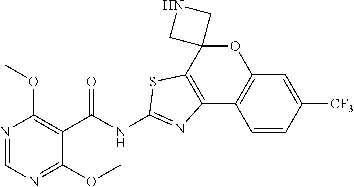 [SMILES: COc1ncnc(OC)c1C(=O)Nc1nc2c(s1)C1(CNC1)Oc1cc(C(F)(F)F)ccc1-2] |
| 68 | 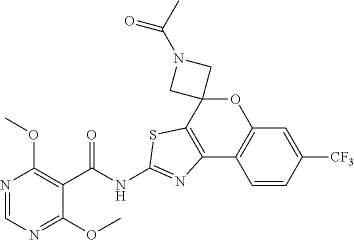 [SMILES: COc1ncnc(OC)c1C(=O)Nc1nc2c(s1)C1(CN(C(C)=O)C1)Oc1cc(C(F)(F)F)ccc1-2] |
| 69 | 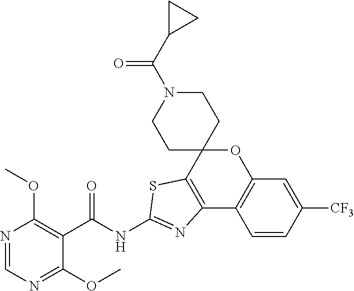 [SMILES: COc1ncnc(OC)c1C(=O)Nc1nc2c(s1)C1(CCN(C(=O)C3CC3)CC1)Oc1cc(C(F)(F)F)ccc1-2] |
| 70 | 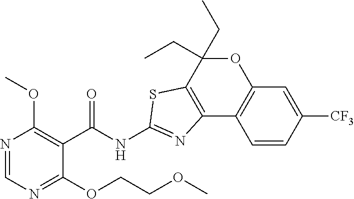 [SMILES: CCC1(CC)Oc2cc(C(F)(F)F)ccc2-c2nc(NC(=O)c3c(OC)ncnc3OCCOC)sc21] |
| 71 | 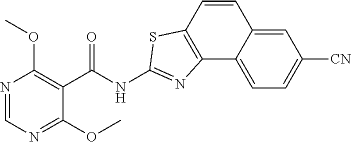 [SMILES: COc1ncnc(OC)c1C(=O)Nc1nc2c(ccc3cc(C#N)ccc32)s1] |
| 72 | 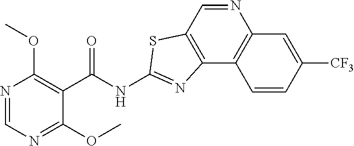 [SMILES: COc1ncnc(OC)c1C(=O)Nc1nc2c(cnc3cc(C(F)(F)F)ccc32)s1] |
| 73 | 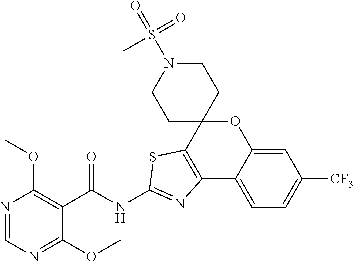 [SMILES: COc1ncnc(OC)c1C(=O)Nc1nc2c(s1)C1(CCN(S(C)(=O)=O)CC1)Oc1cc(C(F)(F)F)ccc1-2] |
| 74 | 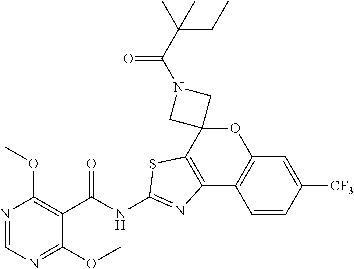 [SMILES: CCC(C)(C)C(=O)N1CC2(C1)Oc1cc(C(F)(F)F)ccc1-c1nc(NC(=O)c3c(OC)ncnc3OC)sc12] |
| 75 | 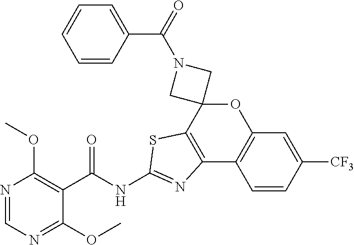 [SMILES: COc1ncnc(OC)c1C(=O)Nc1nc2c(s1)C1(CN(C(=O)c3ccccc3)C1)Oc1cc(C(F)(F)F)ccc1-2] |
| 76 | 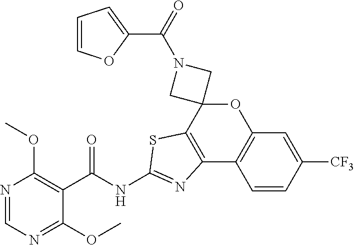 [SMILES: COc1ncnc(OC)c1C(=O)Nc1nc2c(s1)C1(CN(C(=O)c3ccco3)C1)Oc1cc(C(F)(F)F)ccc1-2] |
| 77 | 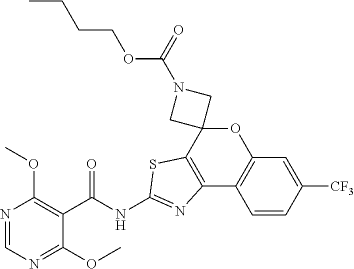 [SMILES: CCCCOC(=O)N1CC2(C1)Oc1cc(C(F)(F)F)ccc1-c1nc(NC(=O)c3c(OC)ncnc3OC)sc12] |
| 78 | 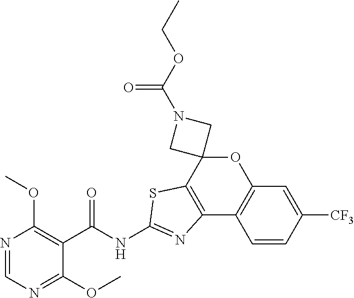 [SMILES: CCOC(=O)N1CC2(C1)Oc1cc(C(F)(F)F)ccc1-c1nc(NC(=O)c3c(OC)ncnc3OC)sc12] |
| 79 | 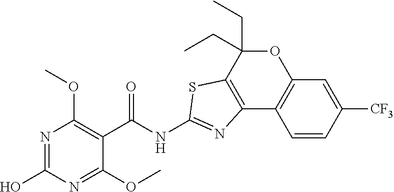 [SMILES: CCC1(CC)Oc2cc(C(F)(F)F)ccc2-c2nc(NC(=O)c3c(OC)nc(O)nc3OC)sc21] |
| 80 | 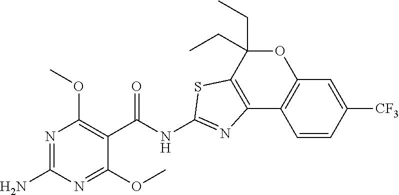 [SMILES: CCC1(CC)Oc2cc(C(F)(F)F)ccc2-c2nc(NC(=O)c3c(OC)nc(N)nc3OC)sc21] |
| 81 | 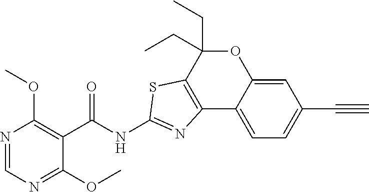 [SMILES: C#Cc1ccc2c(c1)OC(CC)(CC)c1sc(NC(=O)c3c(OC)ncnc3OC)nc1-2] |
| 82 | 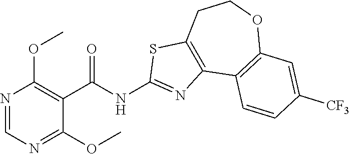 [SMILES: COc1ncnc(OC)c1C(=O)Nc1nc2c(s1)CCOc1cc(C(F)(F)F)ccc1-2] |
| 83 | 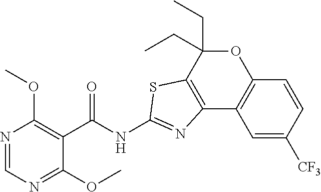 [SMILES: CCC1(CC)Oc2ccc(C(F)(F)F)cc2-c2nc(NC(=O)c3c(OC)ncnc3OC)sc21] |
| 84 | 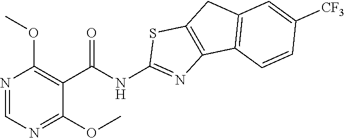 [SMILES: COc1ncnc(OC)c1C(=O)Nc1nc2c(s1)Cc1cc(C(F)(F)F)ccc1-2] |
| 85 | 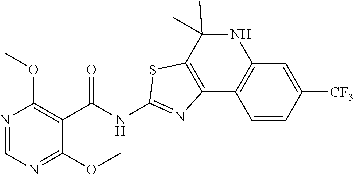 [SMILES: COc1ncnc(OC)c1C(=O)Nc1nc2c(s1)C(C)(C)Nc1cc(C(F)(F)F)ccc1-2] |
| 86 | 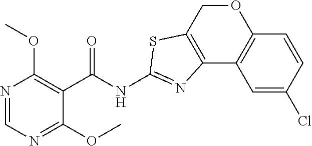 [SMILES: COc1ncnc(OC)c1C(=O)Nc1nc2c(s1)COc1ccc(Cl)cc1-2] |
| 87 | 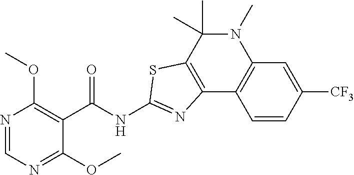 [SMILES: COc1ncnc(OC)c1C(=O)Nc1nc2c(s1)C(C)(C)N(C)c1cc(C(F)(F)F)ccc1-2] |
| 88 | 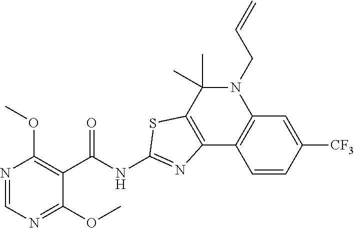 [SMILES: C=CCN1c2cc(C(F)(F)F)ccc2-c2nc(NC(=O)c3c(OC)ncnc3OC)sc2C1(C)C] |
| 89 | 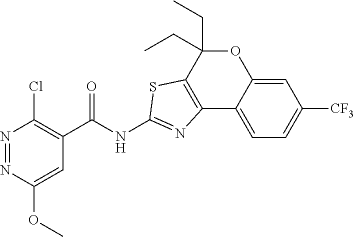 [SMILES: CCC1(CC)Oc2cc(C(F)(F)F)ccc2-c2nc(NC(=O)c3cc(OC)nnc3Cl)sc21] |
| 90 | 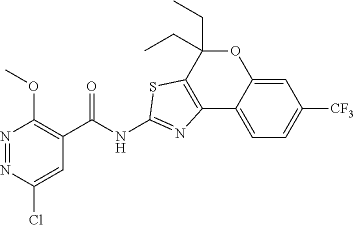 [SMILES: CCC1(CC)Oc2cc(C(F)(F)F)ccc2-c2nc(NC(=O)c3cc(Cl)nnc3OC)sc21] |
| 91 | 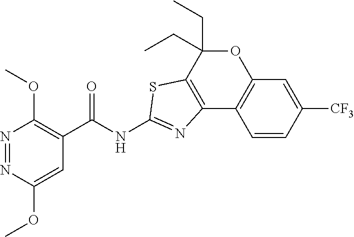 [SMILES: CCC1(CC)Oc2cc(C(F)(F)F)ccc2-c2nc(NC(=O)c3cc(OC)nnc3OC)sc21] |
| 92 | 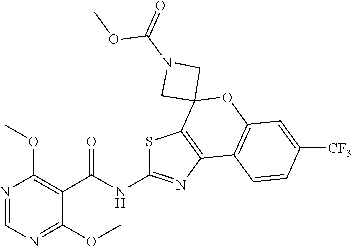 [SMILES: COC(=O)N1CC2(C1)Oc1cc(C(F)(F)F)ccc1-c1nc(NC(=O)c3c(OC)ncnc3OC)sc12] |
| 93 | 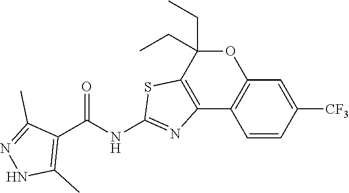 [SMILES: CCC1(CC)Oc2cc(C(F)(F)F)ccc2-c2nc(NC(=O)c3c(C)n[nH]c3C)sc21] |
| 94 | 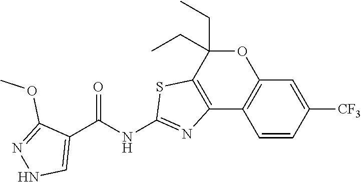 [SMILES: CCC1(CC)Oc2cc(C(F)(F)F)ccc2-c2nc(NC(=O)c3c[nH]nc3OC)sc21] |
| 95 | 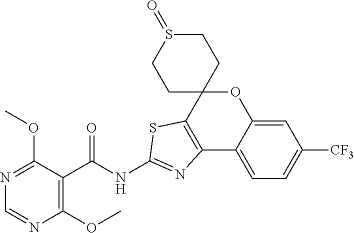 [SMILES: COc1ncnc(OC)c1C(=O)Nc1nc2c(s1)C1(CCS(=O)CC1)Oc1cc(C(F)(F)F)ccc1-2] |
| 96 | 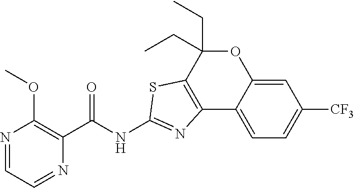 [SMILES: CCC1(CC)Oc2cc(C(F)(F)F)ccc2-c2nc(NC(=O)c3nccnc3OC)sc21] |
| 97 | 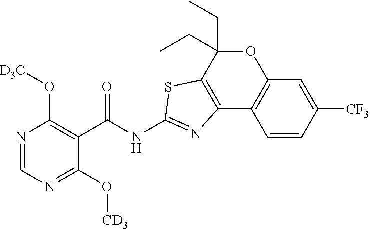 [SMILES: [2H]C([2H])([2H])Oc1ncnc(OC([2H])([2H])[2H])c1C(=O)Nc1nc2c(s1)C(CC)(CC)Oc1cc(C(F)(F)F)ccc1-2] |
| 98 | 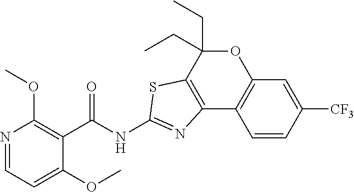 [SMILES: CCC1(CC)Oc2cc(C(F)(F)F)ccc2-c2nc(NC(=O)c3c(OC)ccnc3OC)sc21] |
| 99 | 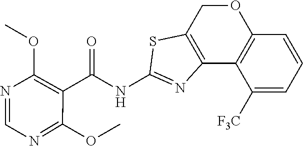 [SMILES: COc1ncnc(OC)c1C(=O)Nc1nc2c(s1)COc1cccc(C(F)(F)F)c1-2] |
| 100 | 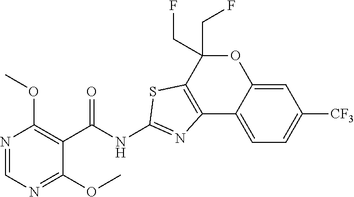 [SMILES: COc1ncnc(OC)c1C(=O)Nc1nc2c(s1)C(CF)(CF)Oc1cc(C(F)(F)F)ccc1-2] |
| 101 | 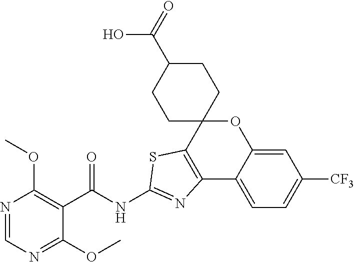 [SMILES: COc1ncnc(OC)c1C(=O)Nc1nc2c(s1)C1(CCC(C(=O)O)CC1)Oc1cc(C(F)(F)F)ccc1-2] |
| 102 | 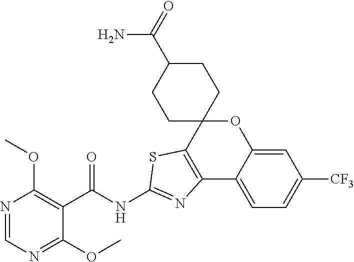 [SMILES: COc1ncnc(OC)c1C(=O)Nc1nc2c(s1)C1(CCC(C(N)=O)CC1)Oc1cc(C(F)(F)F)ccc1-2] |
| 103 | 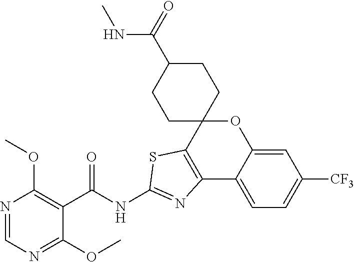 [SMILES: CNC(=O)C1CCC2(CC1)Oc1cc(C(F)(F)F)ccc1-c1nc(NC(=O)c3c(OC)ncnc3OC)sc12] |
| 104 | 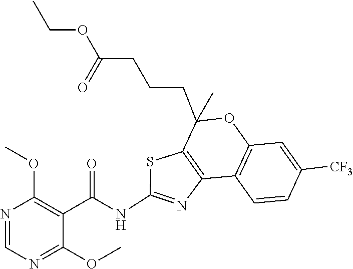 [SMILES: CCOC(=O)CCCC1(C)Oc2cc(C(F)(F)F)ccc2-c2nc(NC(=O)c3c(OC)ncnc3OC)sc21] |
| 105 | 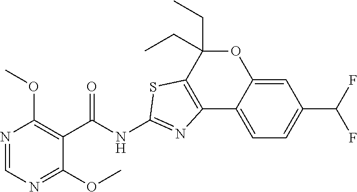 [SMILES: CCC1(CC)Oc2cc(C(F)F)ccc2-c2nc(NC(=O)c3c(OC)ncnc3OC)sc21] |
| 106 | 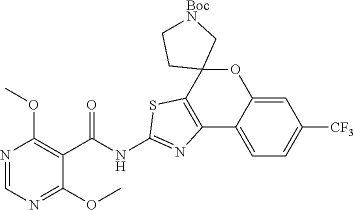 [SMILES: COc1ncnc(OC)c1C(=O)Nc1nc2c(s1)C1(CCN(C(=O)OC(C)(C)C)C1)Oc1cc(C(F)(F)F)ccc1-2] |
| 107 | 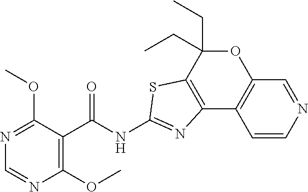 [SMILES: CCC1(CC)Oc2cnccc2-c2nc(NC(=O)c3c(OC)ncnc3OC)sc21] |
| 108 | 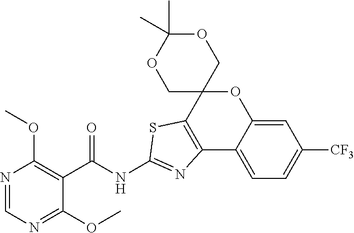 [SMILES: COc1ncnc(OC)c1C(=O)Nc1nc2c(s1)C1(COC(C)(C)OC1)Oc1cc(C(F)(F)F)ccc1-2] |
| 109 | 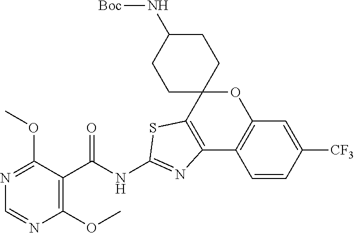 [SMILES: COc1ncnc(OC)c1C(=O)Nc1nc2c(s1)C1(CCC(NC(=O)OC(C)(C)C)CC1)Oc1cc(C(F)(F)F)ccc1-2] |
| 110 | 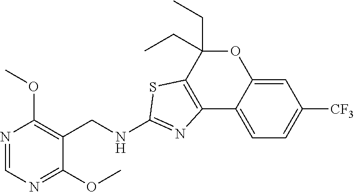 [SMILES: CCC1(CC)Oc2cc(C(F)(F)F)ccc2-c2nc(NCc3c(OC)ncnc3OC)sc21] |
| 111 | 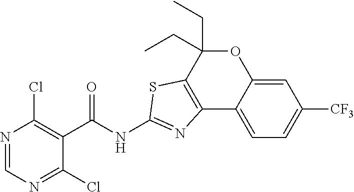 [SMILES: CCC1(CC)Oc2cc(C(F)(F)F)ccc2-c2nc(NC(=O)c3c(Cl)ncnc3Cl)sc21] |
| 112 | 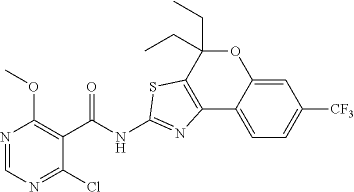 [SMILES: CCC1(CC)Oc2cc(C(F)(F)F)ccc2-c2nc(NC(=O)c3c(Cl)ncnc3OC)sc21] |
| 113 | 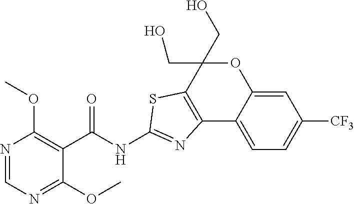 [SMILES: COc1ncnc(OC)c1C(=O)Nc1nc2c(s1)C(CO)(CO)Oc1cc(C(F)(F)F)ccc1-2] |
| 114 | 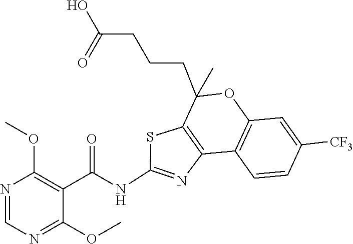 [SMILES: COc1ncnc(OC)c1C(=O)Nc1nc2c(s1)C(C)(CCCC(=O)O)Oc1cc(C(F)(F)F)ccc1-2] |
| 115 | 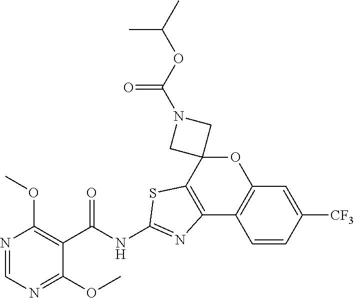 [SMILES: COc1ncnc(OC)c1C(=O)Nc1nc2c(s1)C1(CN(C(=O)OC(C)C)C1)Oc1cc(C(F)(F)F)ccc1-2] |
| 116 | 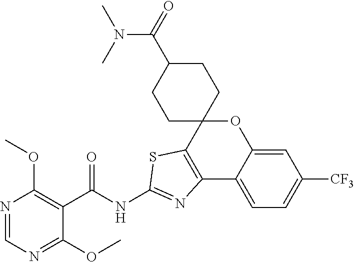 [SMILES: COc1ncnc(OC)c1C(=O)Nc1nc2c(s1)C1(CCC(C(=O)N(C)C)CC1)Oc1cc(C(F)(F)F)ccc1-2] |
| 117 | 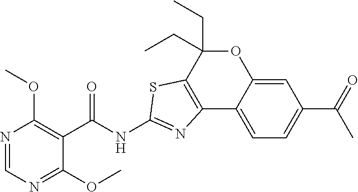 [SMILES: CCC1(CC)Oc2cc(C(C)=O)ccc2-c2nc(NC(=O)c3c(OC)ncnc3OC)sc21] |
| 118 | 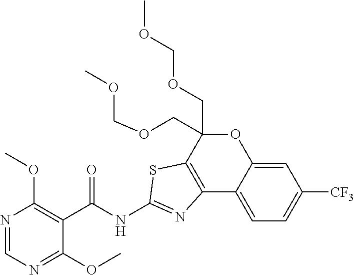 [SMILES: COCOCC1(COCOC)Oc2cc(C(F)(F)F)ccc2-c2nc(NC(=O)c3c(OC)ncnc3OC)sc21] |
| 119 | 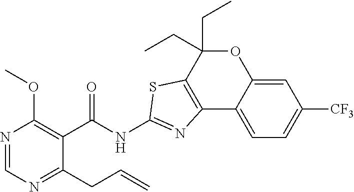 [SMILES: C=CCc1ncnc(OC)c1C(=O)Nc1nc2c(s1)C(CC)(CC)Oc1cc(C(F)(F)F)ccc1-2] |
| 120 | 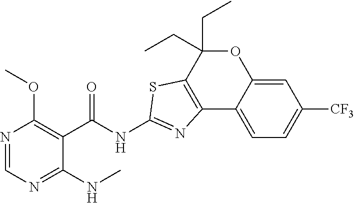 [SMILES: CCC1(CC)Oc2cc(C(F)(F)F)ccc2-c2nc(NC(=O)c3c(NC)ncnc3OC)sc21] |
| 121 | 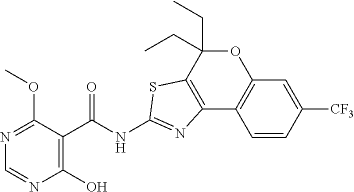 [SMILES: CCC1(CC)Oc2cc(C(F)(F)F)ccc2-c2nc(NC(=O)c3c(O)ncnc3OC)sc21] |
| 122 | 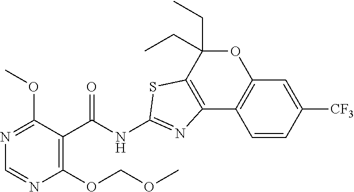 [SMILES: CCC1(CC)Oc2cc(C(F)(F)F)ccc2-c2nc(NC(=O)c3c(OC)ncnc3OCOC)sc21] |
| 123 | 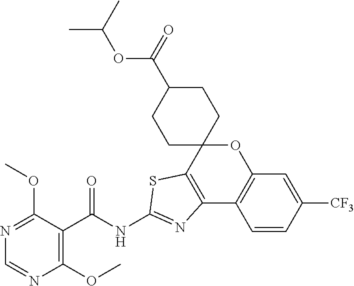 [SMILES: COc1ncnc(OC)c1C(=O)Nc1nc2c(s1)C1(CCC(C(=O)OC(C)C)CC1)Oc1cc(C(F)(F)F)ccc1-2] |
| 124 | 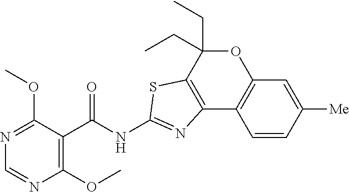 [SMILES: CCC1(CC)Oc2cc(C)ccc2-c2nc(NC(=O)c3c(OC)ncnc3OC)sc21] |
| 125 | 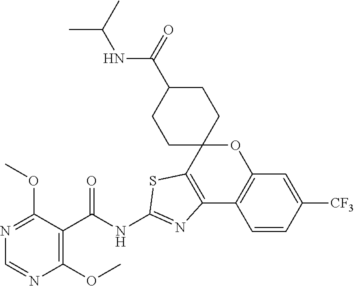 [SMILES: COc1ncnc(OC)c1C(=O)Nc1nc2c(s1)C1(CCC(C(=O)NC(C)C)CC1)Oc1cc(C(F)(F)F)ccc1-2] |
| 126 | 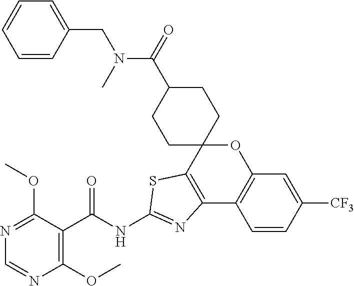 [SMILES: COc1ncnc(OC)c1C(=O)Nc1nc2c(s1)C1(CCC(C(=O)N(C)Cc3ccccc3)CC1)Oc1cc(C(F)(F)F)ccc1-2] |
| 127 | 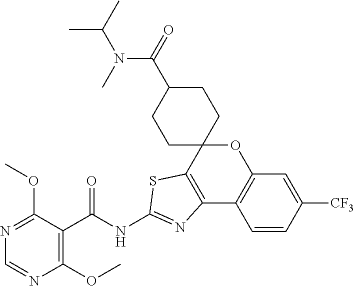 [SMILES: COc1ncnc(OC)c1C(=O)Nc1nc2c(s1)C1(CCC(C(=O)N(C)C(C)C)CC1)Oc1cc(C(F)(F)F)ccc1-2] |
| 128 | 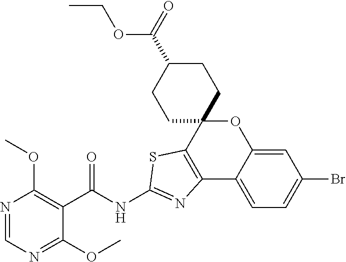 |
| 129 | 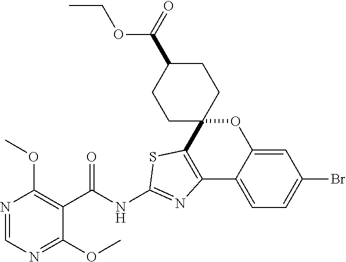 |
| 130 | 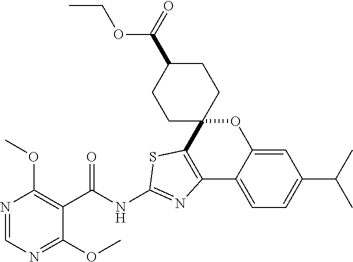 |
| 131 | 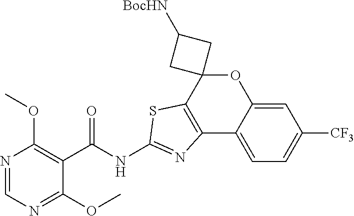 [SMILES: COc1ncnc(OC)c1C(=O)Nc1nc2c(s1)C1(CC(NC(=O)OC(C)(C)C)C1)Oc1cc(C(F)(F)F)ccc1-2] |
| 132 | 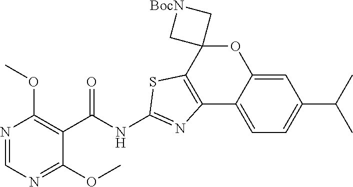 [SMILES: COc1ncnc(OC)c1C(=O)Nc1nc2c(s1)C1(CN(C(=O)OC(C)(C)C)C1)Oc1cc(C(C)C)ccc1-2] |
| 133 | 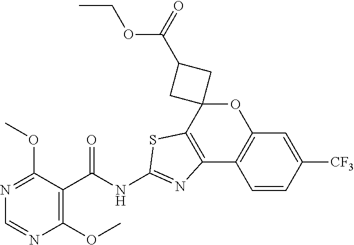 [SMILES: CCOC(=O)C1CC2(C1)Oc1cc(C(F)(F)F)ccc1-c1nc(NC(=O)c3c(OC)ncnc3OC)sc12] |
| 134 | 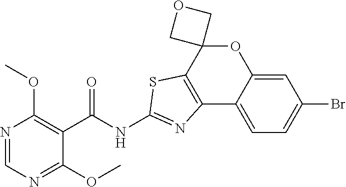 [SMILES: COc1ncnc(OC)c1C(=O)Nc1nc2c(s1)C1(COC1)Oc1cc(Br)ccc1-2] |
| 135 | 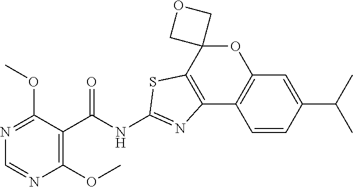 [SMILES: COc1ncnc(OC)c1C(=O)Nc1nc2c(s1)C1(COC1)Oc1cc(C(C)C)ccc1-2] |
| 136 | 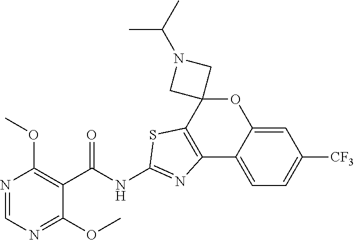 [SMILES: COc1ncnc(OC)c1C(=O)Nc1nc2c(s1)C1(CN(C(C)C)C1)Oc1cc(C(F)(F)F)ccc1-2] |
| 137 | 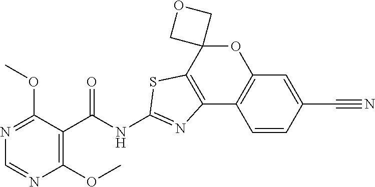 [SMILES: COc1ncnc(OC)c1C(=O)Nc1nc2c(s1)C1(COC1)Oc1cc(C#N)ccc1-2] |
| 138 | 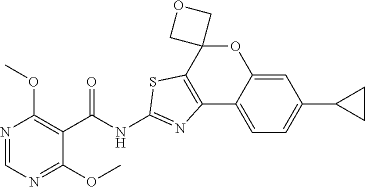 [SMILES: COc1ncnc(OC)c1C(=O)Nc1nc2c(s1)C1(COC1)Oc1cc(C3CC3)ccc1-2] |
| 139 | 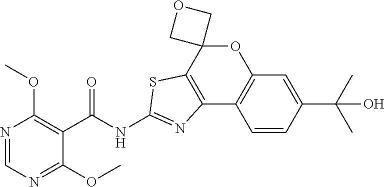 [SMILES: COc1ncnc(OC)c1C(=O)Nc1nc2c(s1)C1(COC1)Oc1cc(C(C)(C)O)ccc1-2] |
| 140 | 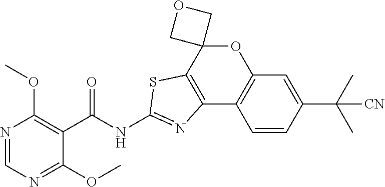 [SMILES: COc1ncnc(OC)c1C(=O)Nc1nc2c(s1)C1(COC1)Oc1cc(C(C)(C)C#N)ccc1-2] |
| 141 | 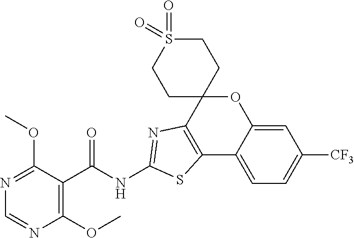 [SMILES: COc1ncnc(OC)c1C(=O)Nc1nc2c(s1)-c1ccc(C(F)(F)F)cc1OC21CCS(=O)(=O)CC1] |
| 142 | 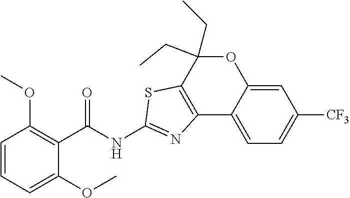 [SMILES: CCC1(CC)Oc2cc(C(F)(F)F)ccc2-c2nc(NC(=O)c3c(OC)cccc3OC)sc21] |
| 143 | 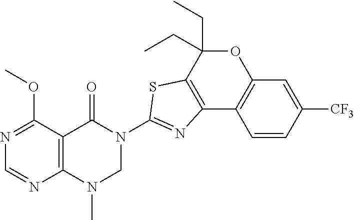 [SMILES: CCC1(CC)Oc2cc(C(F)(F)F)ccc2-c2nc(N3CN(C)c4ncnc(OC)c4C3=O)sc21] |
| 144 | 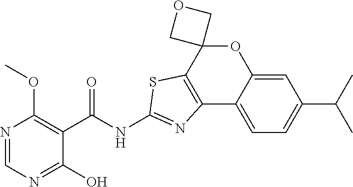 [SMILES: COc1ncnc(O)c1C(=O)Nc1nc2c(s1)C1(COC1)Oc1cc(C(C)C)ccc1-2] |
| 145 | 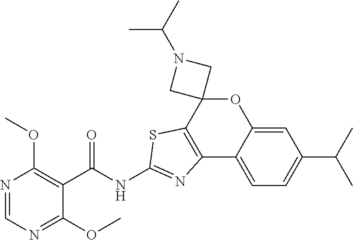 [SMILES: COc1ncnc(OC)c1C(=O)Nc1nc2c(s1)C1(CN(C(C)C)C1)Oc1cc(C(C)C)ccc1-2] |
| 146 | 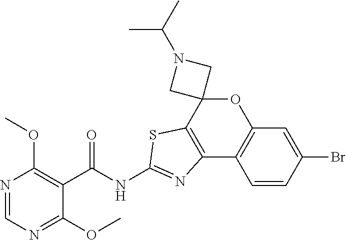 [SMILES: COc1ncnc(OC)c1C(=O)Nc1nc2c(s1)C1(CN(C(C)C)C1)Oc1cc(Br)ccc1-2] |
| 147 | 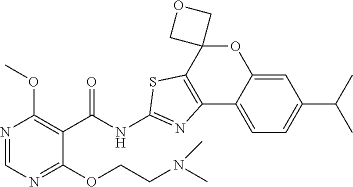 [SMILES: COc1ncnc(OCCN(C)C)c1C(=O)Nc1nc2c(s1)C1(COC1)Oc1cc(C(C)C)ccc1-2] |
| 148 | 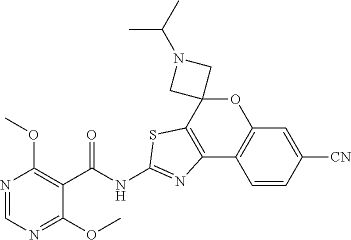 [SMILES: COc1ncnc(OC)c1C(=O)Nc1nc2c(s1)C1(CN(C(C)C)C1)Oc1cc(C#N)ccc1-2] |
| 149 | 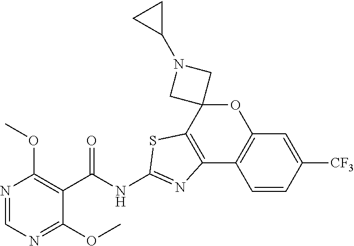 [SMILES: COc1ncnc(OC)c1C(=O)Nc1nc2c(s1)C1(CN(C3CC3)C1)Oc1cc(C(F)(F)F)ccc1-2] |
| 150 | 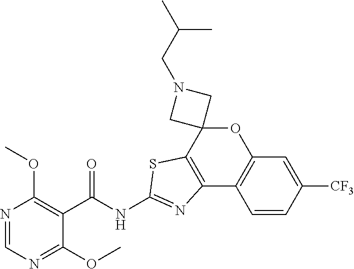 [SMILES: COc1ncnc(OC)c1C(=O)Nc1nc2c(s1)C1(CN(CC(C)C)C1)Oc1cc(C(F)(F)F)ccc1-2] |
| 151 | 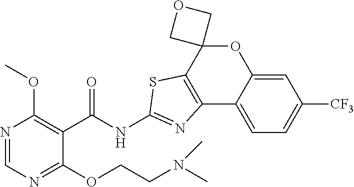 [SMILES: COc1ncnc(OCCN(C)C)c1C(=O)Nc1nc2c(s1)C1(COC1)Oc1cc(C(F)(F)F)ccc1-2] |
| 152 | 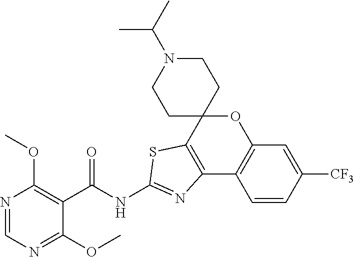 [SMILES: COc1ncnc(OC)c1C(=O)Nc1nc2c(s1)C1(CCN(C(C)C)CC1)Oc1cc(C(F)(F)F)ccc1-2] |
| 153 | 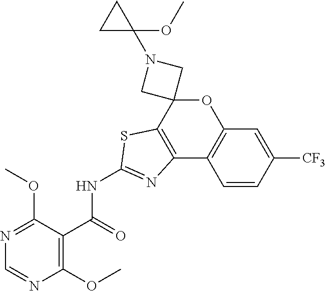 [SMILES: COc1ncnc(OC)c1C(=O)Nc1nc2c(s1)C1(CN(C3(OC)CC3)C1)Oc1cc(C(F)(F)F)ccc1-2] |
| 154 | 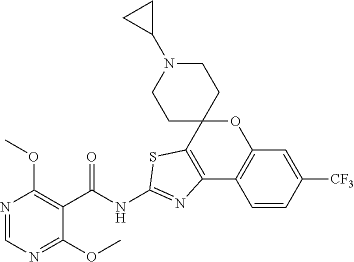 [SMILES: COc1ncnc(OC)c1C(=O)Nc1nc2c(s1)C1(CCN(C3CC3)CC1)Oc1cc(C(F)(F)F)ccc1-2] |
| 155 | 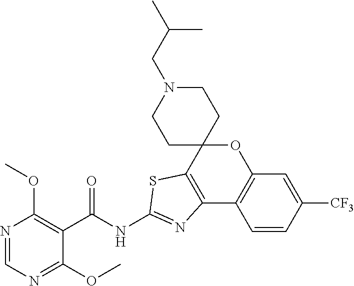 [SMILES: COc1ncnc(OC)c1C(=O)Nc1nc2c(s1)C1(CCN(CC(C)C)CC1)Oc1cc(C(F)(F)F)ccc1-2] |
| 156 | 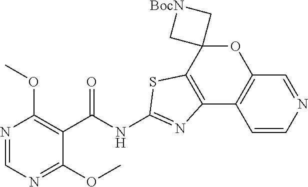 [SMILES: COc1ncnc(OC)c1C(=O)Nc1nc2c(s1)C1(CN(C(=O)OC(C)(C)C)C1)Oc1cnccc1-2] |
| 157 | 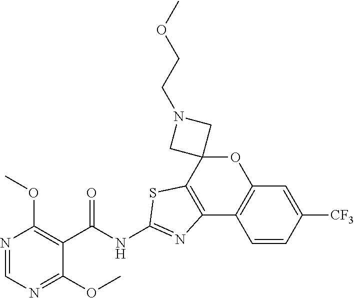 [SMILES: COCCN1CC2(C1)Oc1cc(C(F)(F)F)ccc1-c1nc(NC(=O)c3c(OC)ncnc3OC)sc12] |
| 158 | 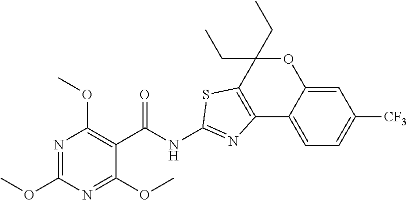 [SMILES: CCC1(CC)Oc2cc(C(F)(F)F)ccc2-c2nc(NC(=O)c3c(OC)nc(OC)nc3OC)sc21] |
| 159 | 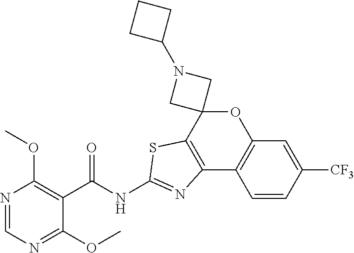 [SMILES: COc1ncnc(OC)c1C(=O)Nc1nc2c(s1)C1(CN(C3CCC3)C1)Oc1cc(C(F)(F)F)ccc1-2] |
| 160 | 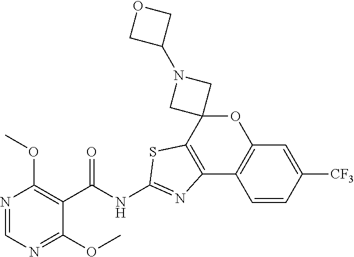 [SMILES: COc1ncnc(OC)c1C(=O)Nc1nc2c(s1)C1(CN(C3COC3)C1)Oc1cc(C(F)(F)F)ccc1-2] |
| 161 | 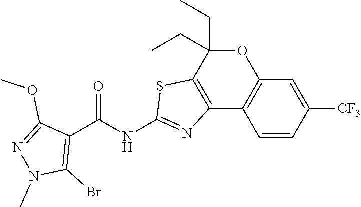 [SMILES: CCC1(CC)Oc2cc(C(F)(F)F)ccc2-c2nc(NC(=O)c3c(OC)nn(C)c3Br)sc21] |
| 162 | 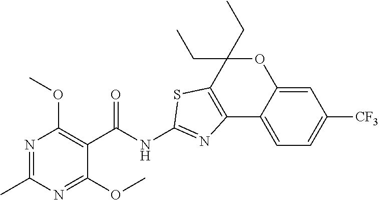 [SMILES: CCC1(CC)Oc2cc(C(F)(F)F)ccc2-c2nc(NC(=O)c3c(OC)nc(C)nc3OC)sc21] |
| 163 | 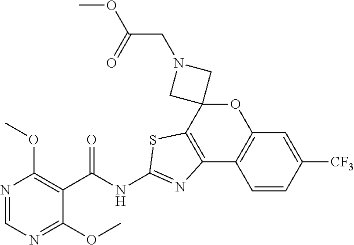 [SMILES: COC(=O)CN1CC2(C1)Oc1cc(C(F)(F)F)ccc1-c1nc(NC(=O)c3c(OC)ncnc3OC)sc12] |
| 164 | 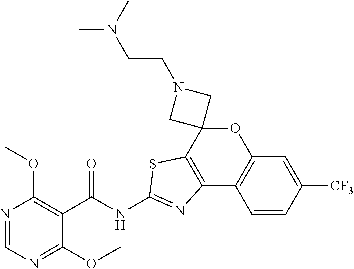 [SMILES: COc1ncnc(OC)c1C(=O)Nc1nc2c(s1)C1(CN(CCN(C)C)C1)Oc1cc(C(F)(F)F)ccc1-2] |
| 165 | 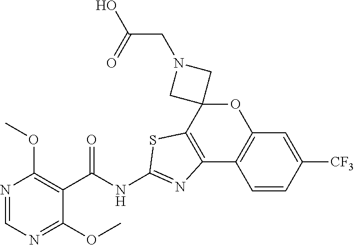 [SMILES: COc1ncnc(OC)c1C(=O)Nc1nc2c(s1)C1(CN(CC(=O)O)C1)Oc1cc(C(F)(F)F)ccc1-2] |
| 166 | 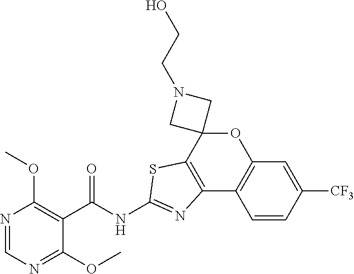 [SMILES: COc1ncnc(OC)c1C(=O)Nc1nc2c(s1)C1(CN(CCO)C1)Oc1cc(C(F)(F)F)ccc1-2] |
| 167 | 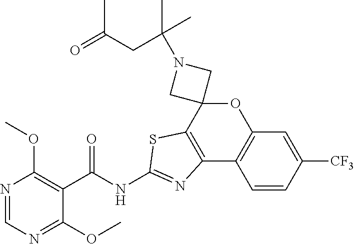 [SMILES: COc1ncnc(OC)c1C(=O)Nc1nc2c(s1)C1(CN(C(C)(C)CC(C)=O)C1)Oc1cc(C(F)(F)F)ccc1-2] |
| 168 | 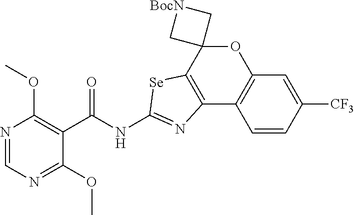 [SMILES: COc1ncnc(OC)c1C(=O)Nc1nc2c([se]1)C1(CN(C(=O)OC(C)(C)C)C1)Oc1cc(C(F)(F)F)ccc1-2] |

### Manufacturing

In some embodiments, the present disclosure provides various technologies, e.g., reagents, intermediates, conditions, etc. for preparing compounds and compositions as described herein. Those skilled in the art appreciate that many technologies are available and can be utilized in accordance with the present disclosure.

As appreciated by those skilled in the art, in chemical reactions various groups, e.g., hydroxyl, amino, carboxyl, etc. may be protected to avoid undesired reactions. Many technologies for protection/deprotection are available to those skilled in the art and may be utilized in accordance with the present disclosure. Certain such technologies are described herein including exemplified in the Examples.

Various chemical reactions are typically performed in a solvent. In some embodiments, a reaction is performed in a single solvent, e.g., DCM, THF, Et₂O, EtOH, toluene, etc. In some embodiments, a reaction is performed in a mixture of two or more solvents. In some embodiments, a solvent is polar. In some embodiments, a solvent is non-polar. In some embodiments, a solvent is protic. In some embodiments, a solvent is non-protic. In some embodiments, a solvent is polar but is not protic. Suitable solvents for various reactions are available to those skilled in the art and can be utilized in accordance with the present disclosure.

In some embodiments, a reaction is conducted under an inert atmosphere, e.g., N₂, Ar, etc. In some embodiments, a reaction is conducted with exposure to air. In some embodiments, a reaction is conducted under anhydrous conditions, e.g., with reagents, solvents, vessels, etc., properly dried. In some embodiments, a reaction is conducted in the presence of significant of water (e.g., about or more than about 0.1, 0.5, or 1 equivalent).

In some embodiments, reactions are performed, or are performed for periods of time, at temperatures that are higher or lower than or about a standard ambient temperature (25° C.). In some embodiments, a reaction temperature is lower than a standard ambient temperature. In some embodiments, a temperature is about or no more than about −78, −60, −50, −40, −30, −20, −10, 0 or 10° C. In some embodiments, a temperature is about or no more than about 10° C. In some embodiments, a temperature is about or no more than about 15° C. In some embodiments, a temperature is about or no more than about 20° C. In some embodiments, a reaction temperature is about a standard ambient temperature. In some embodiments, a reaction temperature is higher than a standard ambient temperature. In some embodiments, a reaction temperature is about or at least about 35, 40, 50, 60, 70, 80, 90, 100, or 100° C. In some embodiments, a reaction comprises refluxing in a boiling solvent system, e.g., in ether, toluene, etc. In some embodiments, temperature changes during a reaction process, e.g., increasing from a lower temperature to a higher temperature, decreasing from a higher temperature to a lower temperature, or both.

In some embodiments, a product is selectively produced over another potential product. In some embodiments, a product is produced with chemoselectivity, stereoselectivity and/or regioselectivity. In some embodiments, a selectivity is presented as a ratio, e.g., of one product over another. In some embodiments, a ratio is about or at least about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 40:1, 50:1, 60:1, 70:1, 80:1, 90:1, 100:1, 200:1, 500:1 or more.

Reactions may be performed for a variety of time lengths. In some embodiments, reactions complete instantly. In some embodiments, reaction times varies from minutes to hours to days, e.g., 5, 10, 15, 20, 30, 45 minutes, or 1, 1.5, 2, 3, 4, 5, 6, 7, 8, 9, 10, 12, 15, 18, 20 or 22 hours, or one or two days or longer. Those skilled in the art can use various technologies to determine when to terminate reactions, e.g., based on consumption of starting materials, products formation, by-products formation, etc.

In some embodiments, the present disclosure provides compounds of high purity. In some embodiments, purity of a compound is or greater than about 85%, 90%, 91%, 92%, 93%, 94%, 95%, 96%, 97%, 98%, 99%, 99.7%, or 99.9%. In some embodiments, purity of a compound is or greater than about 80%. In some embodiments, purity of a compound is or greater than about 85%. In some embodiments, purity of a compound is or greater than about 90%. In some embodiments, purity of a compound is or greater than about 95%. In some embodiments, purity of a compound is or greater than about 96%. In some embodiments, purity of a compound is or greater than about 97%. In some embodiments, purity of a compound is or greater than about 98%. In some embodiments, purity of a compound is or greater than about 99%. In some embodiments, purity of a compound is or greater than about 99.5%. In some embodiments, purity of a compound is or greater than about 99.7%. In some embodiments, purity of a compound is or greater than about 99.9%.

In some embodiments, the present disclosure provides compounds of high stereochemical purity. In some embodiments, stereochemical purity of a compound is or greater than about 85%, 90%, 91%, 92%, 93%, 94%, 95%, 96%, 97%, 98%, 99%, 99.7%, or 99.9%. In some embodiments, stereochemical purity of a compound is or greater than about 80%. In some embodiments, stereochemical purity of a compound is or greater than about 85%. In some embodiments, stereochemical purity of a compound is or greater than about 90%. In some embodiments, stereochemical purity of a compound is or greater than about 95%. In some embodiments, stereochemical purity of a compound is or greater than about 96%. In some embodiments, stereochemical purity of a compound is or greater than about 97%. In some embodiments, stereochemical purity of a compound is or greater than about 98%. In some embodiments, stereochemical purity of a compound is or greater than about 99%. In some embodiments, stereochemical purity of a compound is or greater than about 99.5%. In some embodiments, stereochemical purity of a compound is or greater than about 99.7%. In some embodiments, stereochemical purity of a compound is or greater than about 99.9%.

In some embodiments, the present disclosure provides compounds of high enantiomeric purity. In some embodiments, enantiomeric purity of a compound is or greater than about 85%, 90%, 91%, 92%, 93%, 94%, 95%, 96%, 97%, 98%, 99%, 99.7%, or 99.9%. In some embodiments, enantiomeric purity of a compound is or greater than about 80%. In some embodiments, enantiomeric purity of a compound is or greater than about 85%. In some embodiments, enantiomeric purity of a compound is or greater than about 90%. In some embodiments, enantiomeric purity of a compound is or greater than about 95%. In some embodiments, enantiomeric purity of a compound is or greater than about 96%. In some embodiments, enantiomeric purity of a compound is or greater than about 97%. In some embodiments, enantiomeric purity of a compound is or greater than about 98%. In some embodiments, enantiomeric purity of a compound is or greater than about 99%. In some embodiments, enantiomeric purity of a compound is or greater than about 99.5%. In some embodiments, enantiomeric purity of a compound is or greater than about 99.7%. In some embodiments, enantiomeric purity of a compound is or greater than about 99.9%.

Stereochemically pure, e.g., enantiomerically pure, compounds and compositions can be prepared utilizing various technologies in accordance with the present disclosure. For example, in some embodiments, they can be prepared through separation including chiral separation; in some embodiments, they can be prepared through stereoselective synthesis.

In some embodiments, a method comprises one or more steps described below, wherein each variable is independently as described herein (“halo” is a halogen). In some embodiments, a method is described below as an example. In some embodiments, the present disclosure provides a compound having a structure of a formula selected below or a salt thereof.

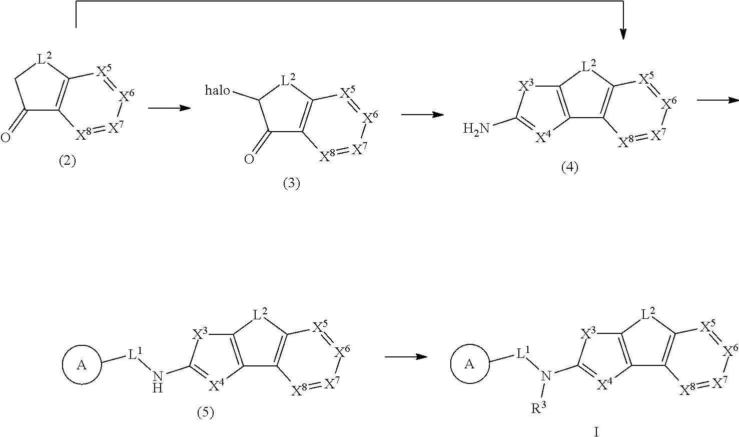

In some embodiments, the present disclosure provides a method for prepare compounds, e.g., compounds of formula I or salts thereof such as compounds of formula C or salts thereof, comprising one or more or all steps illustrated above as an example, wherein each variable is independently as described herein. In some embodiments, contacting a compound having the structure of formula (2) or a salt thereof with halogen provides a compound having the structure of formula (3) or a salt thereof. In some embodiments, a halogen is Cl. In some embodiments, a halogen is Br. In some embodiments, a halogen is I. In some embodiments, a compound having the structure of (3) or a salt thereof reacts with a suitable compound, e.g., a thiourea or a salt thereof, a selenourea or a salt thereof, etc., to provide a compound having the structure of formula (4) or a salt thereof. In some embodiments, a reaction from (3) to (4) is carried out in a solvent system comprising a protic solvent, e.g., an alcohol. In some embodiments, it is carried out in methanol. In some embodiments, a reaction from (3) to (4) is carried out in ethanol. In some embodiments, a reaction from (3) to (4) is carried out or comprises reacting in a temperature of about ambient temperature. In some embodiments, a reaction from (3) to (4) is carried out or comprises reacting in a temperature of about 20-25° C. In some embodiments, a reaction from (3) to (4) is carried out or comprises reacting in a temperature higher than about 25° C. In some embodiments, a reaction from (3) to (4) is carried out or comprises reacting in a temperature of about 40° C. or higher. In some embodiments, it is carried out or comprises reacting at about 40° C. In some embodiments, a reaction from (3) to (4) is carried out or comprises reacting between about 60° C. and about 70° C., optionally with stirring. In some embodiments, it is carried out or comprises reacting at about 60° C. In some embodiments, it is carried out or comprises reacting at about 70° C. In some embodiments, it is carried out or comprises reacting at about 100° C. In some embodiments, from a compound of formula (2) or a salt thereof to a compound of formula (4) or a salt thereof is a one-pot reaction. In some embodiments, a method comprises contacting a compound of formula (2) or a salt thereof with a thiourea or a salt thereof and a halogenating reagent (e.g., a halogen agent such as Br₂, I₂, etc.). In some embodiments, a method comprises contacting a compound of formula (2) or a salt thereof with a selenourea or a salt thereof and a halogenating reagent (e.g., a halogen agent such as Br₂, I₂, etc.). As appreciated by those skilled in the art, many reactions are stirred or otherwise mixed. In some embodiments, a compound having the structure of formula (4) or a salt thereof is utilized to provide a compound having the structure of formula (5) or a salt thereof. In some embodiments, a reaction from (4) to (5) is carried out in DCM. In some embodiments, a reaction from (4) to (5) is carried out in DMF. In some embodiments, a reaction from (4) to (5) is carried out or comprises reacting in a temperature of about ambient temperature. In some embodiments, a reaction from (4) to (5) is carried out or comprises reacting in a temperature of about 20-25° C. In some embodiments, a reaction from (4) to (5) is carried out or comprises reacting in a temperature higher than about 25° C. In some embodiments, a reaction from (4) to (5) is carried out or comprises reacting at above 40° C. or higher. In some embodiments, it is carried out or comprises reacting at about 40° C. In some embodiments, it is carried out or comprises reacting at about 60° C. In some embodiments, it is carried out or comprises reacting at about 70° C. In some embodiments, a reaction from (3) to (4) is carried out or comprises reacting between about 60° C. and about 70° C., optionally with stirring. In some embodiments, R³ is —H, and a compound having the structure of formula (5) or a salt thereof is a compound having the structure of formula I or a salt thereof. In some embodiments, a compound having the structure of formula (5) or a salt thereof is reacted with a compound of formula R³-LG or a salt thereof, wherein LG is a leaving group, to provide a compound having the structure of formula I or a salt thereof. Various leaving groups may be utilized in accordance with the present disclosure. For example, in some embodiments, a leaving group is a halogen. In some embodiments, LG is Cl. In some embodiments, LG is Br. In some embodiments, LG is I. In some embodiments, LG is —S(O)₂R wherein R is as described herein and is not H. In some embodiments, R is optionally substituted C₁₋₆ aliphatic. In some embodiments, R is optionally substituted phenyl. Many suitable conditions may be utilized in accordance with the present disclosure. For example, in some embodiments, a condition is an alkylation condition. In some embodiments, a reaction is performed in the presence of a base. In some embodiments, a base is NaH. In some embodiments, a reaction from (5) to I is carried out in DMF. In some embodiments, a reaction from (5) to I is carried out in DMSO. In some embodiments, a reaction from (5) to I is carried out in THF.

In some embodiments, a compound having the structure of formula (1) or a salt thereof is contacted with a ketone or aldehyde to provide a compound having the structure of formula (2) or a salt thereof:

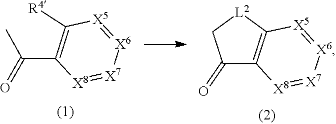

wherein R⁴′ is R⁴ as described herein, and each other variable is independently as described herein. In some embodiments, R⁴′ is —OH. In some embodiments, R⁴′ is —SH.

In some embodiments, the present disclosure provides a method, comprising:
reacting a compound having the structure of formula (5):

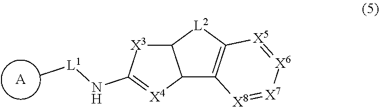

or a salt thereof to provide a compound having the structure of formula I:

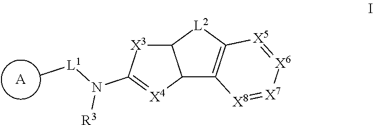

or a salt thereof, wherein each variable is independently as described herein.

In some embodiments, a method comprises contacting a compound having the structure of formula (5) or a salt thereof with a compound having the structure of R³-LG or a salt thereof to provide a compound having the structure of formula I or a salt thereof, wherein each variable is independently as described herein. In some embodiments, contacting is performed under an alkylation condition. In some embodiments, contacting is performed in the presence of a base.

In some embodiments, L¹ in formula (5) is —C(O)— and L¹ in formula I is —CH₂—. In some embodiments, a method comprises contacting a compound having the structure of formula (5) or a salt thereof with a reducing agent to provide a compound having the structure of formula I or a salt thereof, wherein each variable is independently as described herein. In some embodiments, a reducing agent is LiAlH₄.

In some embodiments, the present disclosure provides a method, comprising:
reacting a compound having the structure of formula (4):

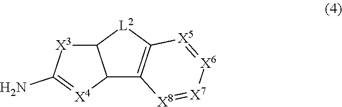

or a salt thereof to provide a compound having the structure of formula (5):

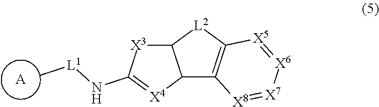

or a salt thereof, wherein each variable is independently as described herein.

In some embodiments, a method comprises contacting a compound having the structure of formula (4) or a salt thereof with a compound having the structure of

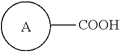

or a salt or an activated derivative thereof, to provide a compound having the structure of formula (5) or a salt thereof, wherein each variable is independently as described herein. In some embodiments, a method comprises contacting a compound having the structure of formula (4) or a salt thereof with a compound having the structure of

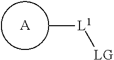

or a salt thereof, to provide a compound having the structure of formula (5) or a salt thereof, wherein each variable is independently as described herein. In some embodiments, L¹ is —C(O)—. In some embodiments, L¹ is —S(O)₂—. In some embodiments, -L¹-LG is an activated-COOH, e.g., —C(O)C₁. In some embodiments, LG is halogen. In some embodiments, LG is —F. In some embodiments, LG is —Cl. In some embodiments, LG is —Br. In some embodiments, LG is or comprises optionally substituted heteroaryl. In some embodiments, a reaction from (4) to (5) is an amidation reaction. Various amidation technologies including activated carboxylic acid derivatives can be utilized in accordance with the present disclosure. In some embodiments, contacting is performed in the presence of a condensing agent, e.g., EFC. In some embodiments, a reagent can promote a reaction, e.g., DMAP is present. In some embodiments, contacting is performed in the presence of a base, e.g., TEA, to neutralize an acid.

In some embodiments, the present disclosure provides a method, comprising:
reacting a compound having the structure of formula (3):

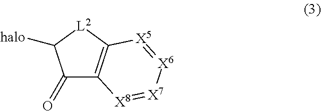

or a salt thereof to provide a compound having the structure of formula (4):

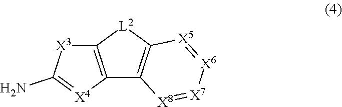

or a salt thereof, wherein each variable is independently as described herein.

In some embodiments, a method comprises contacting a compound having the structure of formula (3) or a salt thereof with a thiourea or a salt thereof to provide a compound having the structure of formula (4) or a salt thereof. In some embodiments, a method comprises contacting a compound having the structure of formula (3) or a salt thereof with a selenourea or a salt thereof to provide a compound having the structure of formula (4) or a salt thereof. In some embodiments, contacting is performed at about room temperature, e.g., at about 25° C. In some embodiments, contacting is performed at an elevated temperature, e.g., at about 100° C. In some embodiments, contacting is performed at two different temperatures, e.g., first at about room temperature for a period of time followed by an elevated temperature, e.g. about 100° C., for a period of time.

In some embodiments, the present disclosure provides a method, comprising:
reacting a compound having the structure of formula (2):

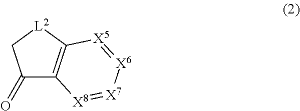

or a salt thereof to provide a compound having the structure of formula (3):

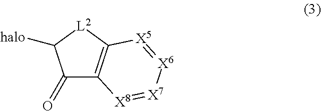

or a salt thereof, wherein each variable is independently as described herein.

In some embodiments, a method comprises contacting a compound having the structure of formula (2) or a salt thereof with a halogenating reagent (e.g., a halogen agent such as Br₂, I₂, etc.).

In some embodiments, the present disclosure provides a method, comprising:
reacting a compound having the structure of formula (2):

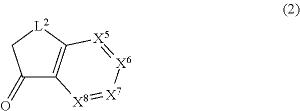

or a salt thereof to provide a compound having the structure of formula (4):

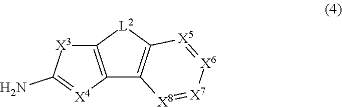

or a salt thereof, wherein each variable is independently as described herein.

In some embodiments, a method comprises contacting a compound having the structure of formula (2) or a salt thereof with a thiourea or a salt thereof and a halogenating agent, e.g., iodine, to provide a compound of formula (4) or a salt thereof. In some embodiments, a method comprises contacting a compound having the structure of formula (2) or a salt thereof with a selenourea or a salt thereof and a halogenating agent, e.g., iodine, to provide a compound of formula (4) or a salt thereof.

In some embodiments, the present disclosure provides a method, comprising:
reacting a compound having the structure of formula (1):

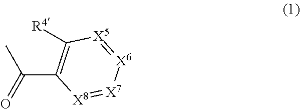

or a salt thereof to provide a compound having the structure of formula (2):

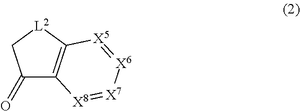

or a salt thereof, wherein each variable is independently as described herein.

In some embodiments, a method comprises contacting a compound having the structure of formula (I) or a salt thereof with a compound having the structure of R⁴C(O)R⁵ or a salt thereof, wherein each of R⁴ and R⁵ are independently as described herein. In some embodiments, one of R⁴ and R⁵ is —H. In some embodiments, R⁴ is R as described herein. In some embodiments, R⁴ is —H. In some embodiments, R⁴ is not —H. In some embodiments, R⁵ is R as described herein. In some embodiments, R⁵ is —H. In some embodiments, R⁵ is not —H. In some embodiments, R⁴ is not —H and R⁵ is not —H. In some embodiments, R⁴ and R⁵ are taken together with the carbon atom to which they are attached to form an optionally substituted ring as described herein. In some embodiments, a method comprises contacting a compound having the structure of formula (I) or a salt thereof with a compound having the structure of

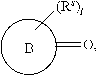

wherein each variable is independently as described herein. In some embodiments, the ═O is part of a ketone group. In some embodiments, contacting is performed in the presence of a base, e.g., pyrrolidine.

### TRPV3

The transient receptor potential vanilloid 3 (TRPV3) has been reported to be a non-selective cation channel, displaying relatively high permeability to calcium. In addition to calcium ions, TRPV3 channels have been reported to be permeable to other cations, e.g., sodium. There are various reports that TRPV3 channels modulate membrane potential by modulating the flux of cations such as calcium and sodium ions. It has been reported that, in some cases, although non-selective cation channels such as TRPV3 modulate, among other things, calcium ion flux, they are mechanistically distinct from voltage-gated calcium channels. For example, there are reports that voltage-gated calcium channels respond to membrane depolarization and open to permit an influx of calcium from the extracellular medium that, in some cases, results in an increase in intracellular calcium levels or concentrations. TRP channels that are non-selective cation channels, according to certain reports, are at least in some cases signal transduction gated, long lasting, and produce more prolonged changes in ion concentration. In some cases, it is reported that these mechanistic differences are accompanied by structural differences among voltage-gated and TRP channels. Many diverse channels have been reported to act to regulate ion flux and membrane potential in various cell types and in response to numerous stimuli. Significant structural, functional, and/or mechanistic differences among different classes of ion channels have been reported.

### Use

It has been reported that as a symptom commonly associated with various forms of dermatitis, such as senile xerosis and atopic dermatitis (AD), chronic itching or pruritus affects millions of people worldwide and has a major impact on their quality of life. In some embodiments, transient receptor potential (TRP) channels are reported or suggested to have key roles in pain and itch perception. In some embodiments, the present disclosure provides technologies for preventing or treating itch. In some embodiments, the present disclosure provides technologies for preventing or treating pruritus. In some embodiments, the present disclosure provides technologies for preventing or treating pain.

According to certain reports, TRP channels are a family of nonselective cation channels that can serve as sensors for various physical and chemical stimuli. Among them, the vanilloid TRP (TRPV) subfamily is reported to comprise six members (TRPV1-TRPV6), four of which (TRPV1-TRPV4) are reported to be activated by heat and therefore in some cases are classified as thermo-TRP channels. TRPV3 has been reported to be a calcium-permeable nonselective cation channel that responds to moderate temperature (~33° C.) and chemical stimuli such as synthetic small molecules (e.g., 2-aminoethoxydiphenyl borate, 2-APB) and certain natural compounds (e.g., camphor). There are reports that TRPV3 is predominantly expressed in epidermal and hair follicle keratinocytes and plays important roles in many aspects of skin functions, including pain and itch sensation, skin barrier formation, and hair growth. Localization of TRPV3 in non-neuronal tissues, e.g., skin, has been reported. In some embodiments, the present disclosure provides technologies for pharmacological modulation of TRPV3 which can provide a therapy to treat diseases impairing skin barrier and optionally have additional benefit for disease states beyond pain. For example, TRPV3 has been reported to play a role in development of dry skin itch, various forms of dermatitis, e.g., atopic dermatitis, rosacea and hair growth disorders. In some embodiments, it is reported that a series of pathogenic mutations of TRPV3, including G573S, G573C, W692G, and G568V, are linked to Olmsted syndrome (OS), a rare congenital disorder characterized by alopecia, keratotic plaque formation and severe itching, which can be due to overactivity of the mutant channels in response to different stimuli. According to certain reports, in contrast to certain other thermo-TRP channels, TRPV3 in some cases can become sensitized rather than desensitized after repetitive stimulation. In some embodiments, the present disclosure provides technologies for inhibition of TRPV3 activity or alleviation of TRPV3-assocaited condition, disorder or disease. In some embodiments, provided compounds that can modulate one or more functions of TRPV3 provide therapeutic benefits in the treatment of various conditions, disorders or diseases including skin conditions, disorders or diseases.

In certain embodiments, the present disclosure provides a method for preventing a condition, disorder or disease, comprising administering to a subject susceptible thereto an effective amount of a provided compound or composition. In certain embodiments, the present disclosure provides a method for treating a condition, disorder or disease, comprising administering to a subject suffering therefrom an effective amount of a provided compound or composition. In certain embodiments, the present disclosure provides a method for preventing a condition, disorder or disease, comprising delivering to a subject susceptible thereto an effective amount of a provided compound or composition. In certain embodiments, the present disclosure provides a method for treating a condition, disorder or disease, comprising delivering to a subject suffering therefrom an effective amount of a provided compound or composition. In some embodiments, a technology comprises administering or delivering to a subject suffering from a condition, disorder or disease, e.g., itch, pain, etc., an effective amount of a provided compound or a pharmaceutical composition as described herein. In some embodiments, a subject suffers from itch. In some embodiments, a subject suffers from chronic itch. In some embodiments, a subject suffers from pain. In some embodiments, a subject suffers from or is diagnosed with dermatitis. In some embodiments, a dermatitis is or comprises senile xerosis. In some embodiments, a dermatitis is or comprises atopic dermatitis. In some embodiments, a condition, disorder or disease impairs skin barrier formation. In some embodiments, a condition, disorder or disease is or comprises slowed or abnormal hair growth. In some embodiments, a condition, disorder or disease is or comprises a hair growth disorder. In some embodiments, a condition, disorder or disease is or comprises dry skin itch. In some embodiments, a condition, disorder or disease is or comprises rosacea. In some embodiments, a condition, disorder or disease is or comprises Olmsted syndrome. In some embodiments, a condition, disorder or disease is or comprises a congenital condition, disorder or disease. In some embodiments, a condition, disorder or disease is or comprises alopecia. In some embodiments, a condition, disorder or disease is or comprises keratotic plaque formation. In some embodiments, a condition, disorder or disease is or comprises severe itching. In some embodiments, a condition, disorder or disease is or comprises a skin condition, disorder or disease. In some embodiments, a condition, disorder or disease is or comprises migraine, arthralgia, cardiac pain arising from an ischemic myocardium, acute pain, chronic pain, nociceptive pain, neuropathic pain, post-operative pain, pain due to neuralgia (e.g., post-herpetic neuralgia, traumatic neuralgia, fibromyalgia, trigeminal neuralgia), pain due to diabetic neuropathy, dental pain, cancer pain, or inflammatory pain conditions (e.g. arthritis and osteoarthritis).

In some embodiments, a condition, disorder or disease is or comprises neuropathic pain, nociceptive pain, dental pain, HIV pain, cardiac pain arising from an ischemic myocardium, pain due to migraine, arthralgia, neuropathies, neurodegeneration, retinopathy, neurotic skin disorder, stroke, urinary bladder hypersensitiveness, urinary incontinence, vulvodynia, gastrointestinal disorders such as irritable bowel syndrome, gastro-esophageal reflux disease, enteritis, ileitis, stomach-duodenal ulcer, inflammatory bowel disease, Crohn's disease, celiac disease, an inflammatory disease such as pancreatitis, a respiratory disorder such as allergic and non-allergic rhinitis, asthma or chronic obstructive pulmonary disease, irritation of skin, eye or mucous membrane, atopic dermatitis, eczema itch, fervescence, muscle spasms, emesis, dyskinesias, depression, Huntington's disease, memory deficits, restricted brain function, amyotrophic lateral sclerosis (ALS), dementia, arthritis, osteoarthritis, diabetes, obesity, urticaria, actinic keratosis, keratocanthoma, alopecia, Meniere's disease, tinnitus, hyperacusis, anxiety disorders, or benign prostate hyperplasia.

In some embodiments, a condition, disorder or disease is or comprises hair loss. In some embodiments, a condition, disorder or disease is hair loss.

In some embodiments, a condition, disorder or disease is or comprises keratoderma. In some embodiments, a condition, disorder or disease is keratoderma

In some embodiments, a condition, disorder or disease is or comprises atopic dermatitis, eczema, sebhorreic eczema, itch, skin inflammation, or psoriasis.

In some embodiments, a condition, disorder or disease is pain. In some embodiments, a condition, disorder or disease is itch. In some embodiments, a condition, disorder or disease is skin inflammation.

In some embodiments, a condition, disorder or disease is or comprises acne. In some embodiments, a condition, disorder or disease is or comprises rosacea.

In some embodiments, a condition, disorder or disease is a TRPV3-associated condition, disorder or disease. In some embodiments, a condition, disorder or disease is associated with TRPV3. In some embodiments, a condition, disorder or disease is associated with TRPV3 activation. In some embodiments, a condition, disorder or disease is modulated by TRPV3.

In certain embodiments, a compound as described herein can be used together with another therapeutic agent as a combination therapy to prevent or treat a condition, disorder or disease. In some embodiments, a condition, disorder or disease is associated with TRPV3 activation. In some embodiments, an another therapeutic agent administered or delivered to a subject can activate TRPV3. In some embodiments, a provided compound can reduce a condition, disorder or disease associated with TRPV3 activation. In some embodiments, a condition, disorder or disease is or comprises concurring TRPV3 related pruritus.

In certain embodiments, the present disclosure provides a method for modulating TRPV3 activity comprising contacting TRPV3 with an effective amount of a compound or a pharmaceutical composition as described herein.

In certain embodiments, the present disclosure provides a method for modulating TRPV3 activity in a system comprising TRPV3, comprising administering or delivering to the system an effective amount of a compound or a pharmaceutical composition as described herein. In certain embodiments, the present disclosure provides a method for modulating a skin function in a system comprising TRPV3, comprising administering or delivering to the system an effective amount of a compound or a pharmaceutical composition as described herein. In some embodiments, a skin function is or comprises pain sensation. In some embodiments, a skin function is or comprises itch sensation. In some embodiments, a skin function is or comprises skin barrier formation. In some embodiments, a skin function is or comprises hair growth.

In some embodiments, a system is or comprises a cell. In some embodiments, a system is or comprises a tissue. In some embodiments, a system is or comprises an organ. In some embodiments, a system is or comprises an organism. In some embodiments, a system is a subject. In some embodiments, a system is an animal. In some embodiments, a system is a human. In some embodiments, a system expresses TRPV3. In some embodiments, a system is or comprises skin. In some embodiments, a system is or comprises non-neuronal cells or tissues. In some embodiments, a system is or comprises epidermal keratinocytes. In some embodiments, a system is or comprises hair follicles. In some embodiments, a method reduces TRPV3 activity level compared absence of a provided compound. In some embodiments, a reduction is about or at least about 20%, 25%, 30%, 40%, 50%, 60%, 70%, 80%, 90%, or 95%.

In some embodiments, a method increases TRPV3 activity level compared absence of a provided compound. In some embodiments, an increase is about or at least about 20%, 25%, 30%, 40%, 50%, 60%, 70%, 80%, 90%, or 95%.

In some embodiments, a compound is administered or delivered as a pharmaceutically acceptable salt form. In some embodiments, a composition is a pharmaceutical composition. In some embodiments, to deliver a provided compound, a prodrug thereof may be administered.

In some embodiments, a compound is utilized in a racemic form. In some embodiments, a composition is a stereorandom mixture of multiple stereoisomers. For example, in some embodiments, a composition is a stereorandom mixture of two enantiomers. In some embodiments, a compound is utilized in a stereochemically pure form as described herein. In some embodiments, a compound is utilized in an enantiomerically pure form. In some embodiments, a composition is enriched for one or more stereoisomers over the others as described herein. In some embodiments, a composition is enriched for an enantiomer as described herein. In some embodiments, a composition is stereochemically pure. In some embodiments, a composition is enantiomerically pure.

In some embodiments, a provided compound is administered or delivered concurrently with another therapeutic agent. In some embodiments, a provided compound is administered or delivered in a single composition with another therapeutic agent. In some embodiments, a provided compound is administered or delivered concurrently with another therapeutic agent but in different compositions. In some embodiments, a provided compound is administered or delivered prior to another therapeutic agent (e.g., about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months prior to another therapeutic agent). In some embodiments, a provided compound is administered or delivered after another therapeutic agent (e.g., about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months after another therapeutic agent). In some embodiments, a provided compound is administered or delivered when a subject is under the therapeutic effect of another therapeutic agent.

In some embodiments, a subject is an adult patient. In some embodiments, a subject is a pediatric patient.

Certain conditions, disorders or diseases, methods and uses are described in WO 2016/160938.

### Pharmaceutical Compositions

In some embodiments, the present disclosure provides a pharmaceutical composition that comprise a provided compound or a pharmaceutically acceptable salt thereof, and a pharmaceutically acceptable carrier. In some embodiments, the present disclosure provides a pharmaceutical composition that can deliver a provided compound or a pharmaceutically acceptable salt thereof, and a pharmaceutically acceptable carrier.

Various technologies, e.g., routes, modes, dosage regimens, etc. may be utilized to administer and/or deliver provided compounds and compositions in accordance with the present disclosure. In some embodiments, a route and/or mode of administration can vary depending upon desired results. One with skill in the art, i.e., a physician, is aware that dosage regimens can be adjusted to provide a desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. In some embodiments, a mode of administration is left to discretion of a practitioner. For example, in some embodiments, a compound or composition is administered or delivered orally. In some embodiments, a compound or composition is administered or delivered topically.

In some embodiments, compounds can be incorporated into and administered as pharmaceutical compositions. Such pharmaceutical compositions are useful for, among other things, administration and delivery to a subject in vivo or ex vivo. In some embodiments, pharmaceutical compositions also contain a pharmaceutically acceptable carrier. In some embodiments, a pharmaceutically acceptable carrier is a pharmaceutical agent that does not itself induce an immune response harmful to the individual receiving a composition, and which may be administered without undue toxicity. Pharmaceutically acceptable carriers (or excipients) include, but are not limited to, liquids such as water, saline, glycerol, sugars and ethanol. Additionally, auxiliary substances, such as wetting or emulsifying agents, pH buffering substances, and the like, may be present in such vehicles.

Compounds in pharmaceutical compositions may be provided as pharmaceutically acceptable salts. In some embodiments, salts can be formed with acids such as hydrochloric acid, sulfuric acid, acetic acid, lactic acid, tartaric acid, malic acid, succinic acid, benzenesulfonic acid, etc. In some embodiments, salts can be formed with bases. In some embodiments, salts are alkali, alkaline earth metal, or ammonium salts, e.g., sodium, calcium, diethanolamine, ethanolamine, trialkylamine salts, etc.

In some embodiments, salts are more soluble in aqueous or other protonic solvents than corresponding, free acid or base forms. In some embodiments, a pharmaceutical composition may be a lyophilized powder. In some embodiments, a pharmaceutical composition comprises a provided compound, e.g., a compound of formula I or a pharmaceutically acceptable salt thereof dissolved in a pharmaceutically acceptable buffer. In some embodiments, a buffer is a saline buffer. In some embodiments, a buffer has a pH around 7.4.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. In some embodiments, pharmaceutical compositions or formulations are tablets (coated or uncoated), capsules (hard or soft), microbeads, powder, granules and/or crystals. Supplementary active compounds (e.g., preservatives, antibacterial, antiviral and antifungal agents) can also be incorporated into pharmaceutical compositions.

Pharmaceutical compositions can be formulated to be compatible with a particular route of administration or delivery as set forth herein or known to one of skill in the art.

In some embodiments, provided compositions are suitable for parenteral administration. In some embodiments, such compositions comprise aqueous and non-aqueous solutions, suspensions or emulsions of active compounds, which preparations are typically sterile and can be isotonic with blood of intended recipients. Examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, a suspension may also contain suitable stabilizers or agents which increase solubility to allow for the preparation of highly concentrated solutions.

Co-solvents and adjuvants may be added to compositions and formulations. Non-limiting examples of co-solvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Various pharmaceutical composition technologies and delivery systems appropriate for compositions, methods and uses of the present disclosure are available (see, e.g., Remington: The Science and Practice of Pharmacy. 23^{rd} Edition., Academic Press, 2020) and can be utilized in accordance with the present disclosure.

In some embodiments, the present disclosure provides methods for delivering provided compounds and compositions into cells, animals or subjects. In some embodiments, such methods include contacting a subject (e.g., a cell or tissue of a subject) with, or administering or delivering to a subject (e.g., a subject such as a mammal or human) a provided compound, e.g., a compound of formula I or a salt thereof, or a composition thereof.

A compound or composition described herein can be administered in a sufficient or effective amount to a subject (or a cell, tissue or organ thereof) in need thereof. Doses can vary and may depend upon the type, onset, progression, severity, frequency, duration, or probability of a condition, disorder or disease to which treatment is directed, a clinical endpoint desired, previous or simultaneous treatments, general health, age, gender, race or immunological competency of a subject and other factors that will be appreciated by a skilled artisan. Dose amount, number, frequency or duration may be proportionally increased or reduced, as indicated by efficacy, any adverse side effects, complications or other risk factors of a treatment or therapy and the status of a subject. A skilled artisan will appreciate factors that may influence dosage and timing required to provide an amount sufficient for providing a therapeutic or prophylactic benefit.

A dose to achieve a therapeutic effect will vary based on several factors including route of administration, amount to achieve a therapeutic effect, specific condition, disorder or disease treated, any host immune response to administered compound or composition, stability of administered compound or composition, etc.

An effective amount or a sufficient amount can be provided in a single administration, may require multiple administrations, and, can be, administered alone or in combination with another composition (e.g., comprising or delivering another therapeutic agent). For example, an amount may be proportionally increased as indicated by the need of a subject, type, status and severity of a condition, disorder or disease treated and/or side effects (if any) of treatment. Amounts considered effective also include amounts that result in a reduction of the use of another treatment, therapeutic regimen or protocol.

In some embodiments, pharmaceutical compositions comprise or deliver active ingredients, e.g., compounds of formula I or pharmaceutically acceptable salts thereof, in effective amounts to achieve intended purposes e.g., therapeutic purposes. Various technologies may be utilized to determine therapeutically effective amounts in accordance with the present disclosure. Therapeutic doses can depend on, among other factors, ages and general conditions of subjects, severity of conditions, disorders or diseases, etc. In some embodiments, therapeutically effective amounts in humans may fall in a relatively broad range that may be determined by medical practitioners based on responses of individual patients.

In some embodiments, methods and uses of the present disclosure include delivery and administration systemically, regionally or locally, or by any route, for example, by injection or infusion or orally. In some embodiments, delivery of a pharmaceutical composition in vivo may generally be accomplished via injection using a conventional syringe, although other delivery methods such as convection-enhanced delivery can also be used In some embodiments, compounds and compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. In some embodiments, modes of administration include oral and pulmonary administration, suppositories, and transdermal applications. Clinicians specializing in treating patients may determine optimal routes for administration of compounds and compositions as described herein. Among other things, the present disclosure provides the following Embodiments:

1. A compound, wherein the compound has the structure of formula I:

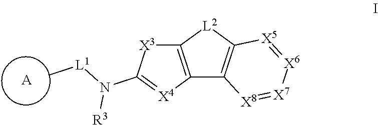

or a salt thereof, wherein:
Ring A is an optionally substituted 5-6 membered aromatic ring having 0-4 heteroatoms, or is

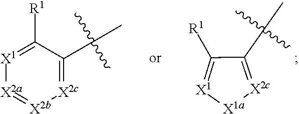

each of X¹, X^{2a}, X^{2b}, and X^{2c} is independently —N═, —C(R²)═ or optionally substituted —CH═;
X^{1a} is —O—, —S—, —N(R′)—, —C(R′)₂″, or optionally substituted —CH₂— or —NH—;
X³ is —O—, —S—, —Se—, —N(R′)— or optionally substituted —NH—;
each of X⁴, X⁵, X⁶, X⁷ and X⁸ is independently —N═, —C(R⁶)═ or optionally substituted —CH═;
each of R¹, R², R^{s} and R⁶ is independently halogen, —CN, —NO₂, -L-R′, —OR′, —N(R′)₂, —N(R′)C(O)OR′, —C(O)R′, —C(O)OR′, —C(O)N(R′)₂, or —OC(O)N(R′)₂;
L¹ is a covalent bond, —C(O)—, —C(S)—, —S(O)₂—, —N(R′)C(O)—, —N(R′)S(O)₂—, —N(R′)C(S)— or

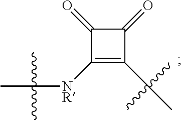

each of R³, R⁴ and R⁵ is independently R′;
each of L and L² is independently optionally substituted C₁₋₃ alkylene, wherein one or more methylene units of the alkylene are optionally and independently replaced with —O—, —S—, C(R⁴)(R⁵)—, —N(R⁴)—, optionally substituted —CH═N—, optionally substituted —CH═CH— or -Cy-;
each -Cy- is independently

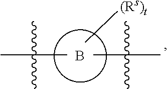

wherein Ring B is an optionally substituted saturated or partially saturated 3-10 membered ring having 0-4 heteroatoms;
t is 0, 1, 2, 3 or 4;
each R′ is independently R, —OR, —C(O)R, —C(O)OR, or —S(O)₂R;
each R is independently hydrogen or an optionally substituted group selected from C₁-C₁₀ aliphatic, C₁-C₁₀ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₁₀ aliphatic, and 5-10 membered heteroaryl having 1-6 heteroatoms-C₁-C₁₀ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

2. A compound, wherein the compound has the structure of

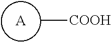

derivative thereof, wherein Ring A is as defined in embodiment 1.

3. The compound of embodiment 2, wherein the compound has the structure of

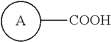

or a salt thereof.

4. A compound, wherein the compound has the structure of

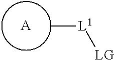

or a salt thereof, wherein Ring A and L¹ are independently as defined in embodiment 1, and LG is a leaving group.

5. The compound of any one of embodiments 1-4, wherein Ring A is optionally substituted phenyl.

6. The compound of any one of embodiments 1-4, wherein Ring A is phenyl.

7. The compound of any one of embodiments 1-4, wherein Ring A is optionally substituted 5-6 membered heteroaryl ring having 1, 2, 3, or 4 heteroatoms.

8. The compound of any one of embodiments 1-4, wherein Ring A is optionally substituted 5 membered heteroaryl ring having 1, 2, 3, or 4 heteroatoms.

9. The compound of any one of embodiments 1-4, wherein Ring A is optionally substituted 5 membered heteroaryl ring having 1 or 2 heteroatoms.

10. The compound of any one of embodiments 1-4, wherein Ring A is optionally substituted 5 membered heteroaryl ring having 2 heteroatoms.

11. The compound of any one of embodiments 1-4, wherein Ring A is optionally substituted 5 membered heteroaryl ring having 2 nitrogen atoms.

12. The compound of any one of embodiments 1-4, wherein Ring A is optionally substituted 6-membered heteroaryl ring having 1, 2, 3, or 4 heteroatoms.

13. The compound of any one of embodiments 1-4, wherein Ring A is optionally substituted 6 membered heteroaryl ring having 1, 2, or 3 heteroatoms.

14. The compound of any one of embodiments 1-4, wherein Ring A is optionally substituted 6 membered heteroaryl ring having 1 or 2 heteroatoms.

15. The compound of any one of embodiments 1-4, wherein Ring A is optionally substituted 6 membered heteroaryl ring having 2 heteroatoms.

16. The compound of any one of embodiments 1-4, wherein Ring A is optionally substituted 6 membered heteroaryl ring having 2 nitrogen atoms.

17. The compound of any one of embodiments 1-4, wherein Ring A is

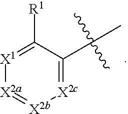

18. The compound of embodiment 17, wherein Ring A is

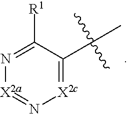

19. The compound of embodiment 17 or 18, wherein Ring A is

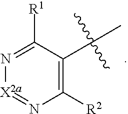

20. The compound of any one of embodiments 17-19, wherein Ring A is

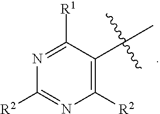

21. The compound of any one of embodiments 17-20, wherein Ring A is

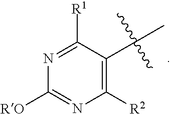

22. The compound of any one of embodiments 17-21, wherein Ring A is

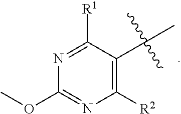

23. The compound of any one of embodiments 17-20, wherein Ring A is

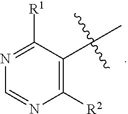

24. The compound of any one of embodiments 17-20, wherein Ring A is

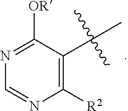

25. The compound of any one of embodiments 17-20, wherein Ring A is

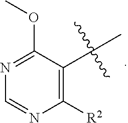

26. The compound of any one of embodiments 17-20, wherein Ring A is

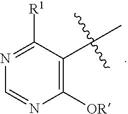

27. The compound of any one of embodiments 17-20, wherein Ring A is

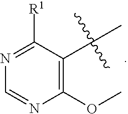

28. The compound of any one of embodiments 17-20, wherein Ring A is

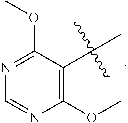 [SMILES: *c1c(OC)ncnc1OC]

29. The compound of embodiment 17, wherein Ring A is

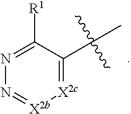

30. The compound of embodiment 29, wherein Ring A is

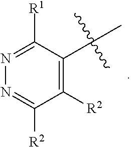

31. The compound of any one of embodiments 29-30, wherein Ring A is

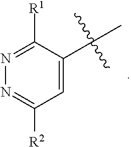

32. The compound of any one of embodiments 29-31, wherein Ring A is

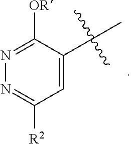

33. The compound of any one of embodiments 29-32, wherein Ring A is

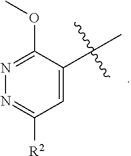

34. The compound of any one of embodiments 29-31, wherein Ring A is

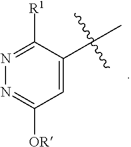

35. The compound of any one of embodiments 29-31, wherein Ring A is

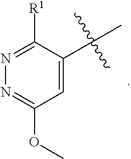

36. The compound of any one of embodiments 29-31, wherein Ring A is

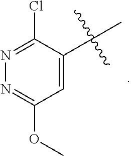 [SMILES: *c1cc(OC)nnc1Cl]

37. The compound of any one of embodiments 29-31, wherein Ring A is

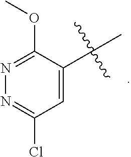 [SMILES: *c1cc(Cl)nnc1OC]

38. The compound of embodiment 17, wherein Ring A is

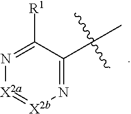

39. The compound of embodiment 38, wherein Ring A is

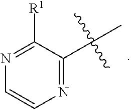

40. The compound of any one of embodiments 38-39, wherein Ring A is

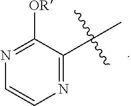

41. The compound of any one of embodiments 38-40, wherein Ring A is

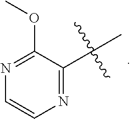 [SMILES: *c1nccnc1OC]

42. The compound of embodiment 17, wherein Ring A is

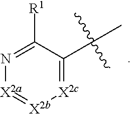

43. The compound of embodiment 42, wherein Ring A is

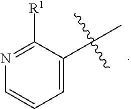

44. The compound of any one of embodiments 42-43, wherein Ring A is

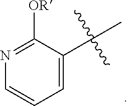

45. The compound of any one of embodiments 42-44, wherein Ring A is

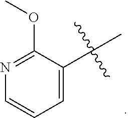 [SMILES: *c1cccnc1OC]

46. The compound of any one of embodiments 1-4, wherein Ring A is

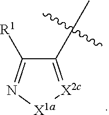

47. The compound of embodiment 46, wherein Ring A is

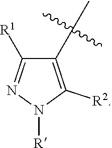

48. The compound of any one of embodiments 46-47, wherein Ring A is

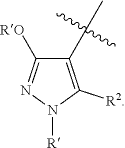

49. The compound of any one of embodiments 46-48, wherein Ring A is

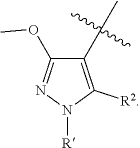

50. The compound of any one of embodiments 46-49, wherein Ring A is

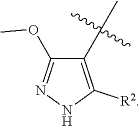

51. The compound of any one of embodiments 46-50, wherein Ring A is

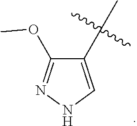 [SMILES: *c1c[nH]nc1OC]

52. The compound of any one of embodiments 46-49, wherein Ring A is

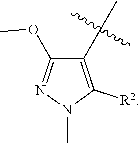

53. The compound of any one of embodiments 46-52, wherein Ring A is

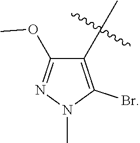 [SMILES: *c1c(OC)nn(C)c1Br]

54. The compound of any one of embodiments 46-47, wherein Ring A is

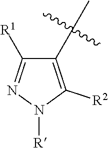

and each of R¹ and R² is independently optionally substituted C₁-C₆ alkyl.

55. The compound of any one of embodiments 46-47, wherein Ring A is

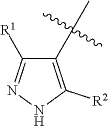

and each of R¹ and R² is independently optionally substituted C₁-C₆ alkyl.

56. The compound of any one of embodiments 46-47, wherein Ring A is

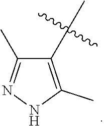

57. A compound, wherein the compound has the structure of

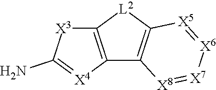

or a salt thereof, wherein each variable is independently as described in any one of the preceding embodiments.

58. A compound, wherein the compound has the structure of

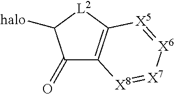

or a salt thereof, wherein halo is a halogen, and each other variable is independently described in any one of the preceding embodiments.

59. A compound, wherein the compound has the structure of

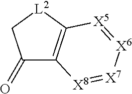

or a salt thereof, wherein each variable is independently as described in any one of the preceding embodiments.

60. The compound of embodiment 58, wherein halo is —Cl.

61. The compound of embodiment 58, wherein halo is —Br.

62. The compound of embodiment 58, wherein halo is —I.

63. The compound of any one of embodiments 1-4, 17 and 46, wherein X¹ is —N═.

64. The compound of any one of embodiments 1-4, 17 and 46, wherein X¹ is —C(R²)—.

65. The compound of any one of embodiments 1-4, 17 and 46, wherein X¹ is optionally substituted —CH═.

66. The compound of any one of embodiments 1-4, 17 and 46, wherein X¹ is —CH═.

67. The compound of any one of embodiments 1-4, 17 and 63-66, wherein X^{2a} is —N═.

68. The compound of any one of embodiments 1-4, 17 and 63-66, wherein X^{2a} is —C(R²)═.

69. The compound of any one of embodiments 1-4, 17 and 63-66, wherein X^{2a} is —C(OR′)═.

70. The compound of any one of embodiments 1-4, 17 and 63-66, wherein X^{2a} is —C(OH)═.

71. The compound of any one of embodiments 1-4, 17 and 63-66, wherein X^{2a} is —C(OR′)═ wherein R′ is optionally substituted C₁-C₆ alkyl.

72. The compound of any one of embodiments 1-4, 17 and 63-66, wherein X^{2a} is —C(OMe)═.

73. The compound of any one of embodiments 1-4, 17 and 63-66, wherein X^{2a} is —C(N(R′)₂)═.

74. The compound of any one of embodiments 1-4, 17 and 63-66, wherein X^{2a} is —C(NH₂)═.

75. The compound of any one of embodiments 1-4, 17 and 63-66, wherein X^{2a} is —C(N(R′)₂)═ wherein each R′ is independently optionally substituted C₁-C₆ alkyl.

76. The compound of any one of embodiments 1-4, 17 and 63-66, wherein X^{2a} is —C(R²)═ wherein R² is optionally substituted C₁-C₆ alkyl. 77. The compound of any one of embodiments 1-4, 17 and 63-66, wherein X^{2a} is optionally substituted —CH═.

78. The compound of any one of embodiments 1-4, 17 and 63-66, wherein X^{2a} is —CH═.

79. The compound of any one of embodiments 1-4, 17 and 63-66, wherein X^{2a} is —C(Me)═.

80. The compound of any one of embodiments 1-4, 17 and 63-79, wherein X^{2b} is —N═.

81. The compound of any one of embodiments 1-4, 17 and 63-79, wherein X^{2b} is —C(R²)═.

82. The compound of any one of embodiments 1-4, 17 and 63-79, wherein X^{2b} is —C(OR′)═.

83. The compound of any one of embodiments 1-4, 17 and 63-79, wherein X^{2b} is —C(OR′)═ wherein R′ is optionally substituted C₁-C₆ alkyl.

84. The compound of any one of embodiments 1-4, 17 and 63-79, wherein X²⁰ is —C(OMe)═.

85. The compound of any one of embodiments 1-4, 17 and 63-79, wherein X^{2b} is optionally substituted CH═.

86. The compound of any one of embodiments 1-4, 17 and 63-79, wherein X^{2b} is —CH═.

87. The compound of any one of embodiments 1-4, 17 and 63-79, wherein X^{2b} is —C(halogen)=.

88. The compound of any one of embodiments 1-4, 17 and 63-79, wherein X^{2b} is —C(Cl)═.

89. The compound of any one of embodiments 1-4, 17, 46 and 63-88, wherein X^{2c} is —N═.

90. The compound of any one of embodiments 1-4, 17, 46 and 63-88, wherein X^{2c} is —C(R²)—.

91. The compound of any one of embodiments 1-4, 17, 46 and 63-88, wherein X^{2c} is optionally substituted —CH═.

92. The compound of any one of embodiments 1-4, 17, 46 and 63-88, wherein X^{2c} is —CH═.

93. The compound of any one of embodiments 1-4, 17, 46 and 63-88, wherein X^{2c} is —C(OR′)═.

94. The compound of any one of embodiments 1-4, 17, 46 and 63-88, wherein X^{2c} is —C(OH)═.

95. The compound of any one of embodiments 1-4, 17, 46 and 63-88, wherein X^{2c} is —C(OR′)═ wherein R′ is optionally substituted C₁-C₆ aliphatic.

96. The compound of any one of embodiments 1-4, 17, 46 and 63-88, wherein X^{2c} is —C(OR′)═ wherein R′ is C₁-C₆ aliphatic.

97. The compound of any one of embodiments 1-4, 17, 46 and 63-88, wherein X^{2c} is —C(OR′)═ wherein R′ is optionally substituted C₁-C₆ alkyl.

98. The compound of any one of embodiments 1-4, 17, 46 and 63-88, wherein X^{2c} is —C(OMe)═.

99. The compound of any one of embodiments 1-4, 17, 46 and 63-88, wherein X^{2c} is

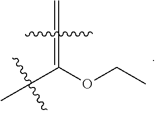 [SMILES: *C(=*)OCC]

100. The compound of any one of embodiments 1-4, 17, 46 and 63-88, wherein X^{2c} is

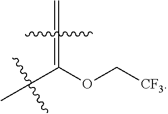 [SMILES: *C(=*)OCC(F)(F)F]

101. The compound of any one of embodiments 1-4, 17, 46 and 63-88, wherein X^{2c} is

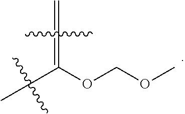 [SMILES: *C(=*)OCOC]

102. The compound of any one of embodiments 1-4, 17, 46 and 63-88, wherein X^{2c} is

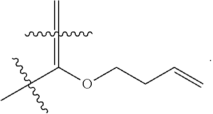 [SMILES: *C(=*)OCCC=C]

103. The compound of any one of embodiments 1-4, 17, 46 and 63-88, wherein X^{2c} is

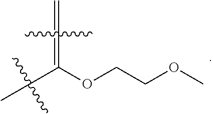 [SMILES: *C(=*)OCCOC]

104. The compound of any one of embodiments 1-4, 17, 46 and 63-88, wherein X^{2c} is

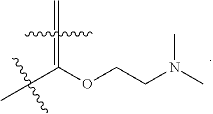 [SMILES: *C(=*)OCCN(C)C]

105. The compound of any one of embodiments 1-4, 17, 46 and 63-88, wherein X^{2c} is —C(R²)═ wherein R² is optionally substituted C₁-C₆ aliphatic.

106. The compound of any one of embodiments 1-4, 17, 46 and 63-88, wherein X^{2c} is —C(R²)═ wherein R² is C₁-C₆ aliphatic.

107. The compound of any one of embodiments 1-4, 17, 46 and 63-88, wherein X^{2c} is —C(R²)═ wherein R² is optionally substituted C₁-C₆ alkyl.

108. The compound of any one of embodiments 1-4, 17, 46 and 63-88, wherein X^{2c} is

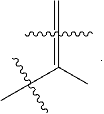 [SMILES: *C(=*)C]

109 The compound of any one of embodiments 1-4, 17, 46 and 63-88, wherein X^{2c} is

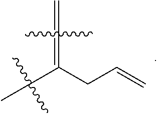 [SMILES: *C(=*)CC=C]

110. The compound of any one of embodiments 1-4, 17, 46 and 63-88, wherein X^{2c} is —C(N(R′)₂)—.

111. The compound of any one of embodiments 1-4, 17, 46 and 63-88, wherein X^{2c} is —C(N(R′)₂)═ wherein each R′ is independently optionally substituted C₁-C₆ alkyl.

112. The compound of any one of embodiments 1-4, 17, 46 and 63-88, wherein X^{2c} is

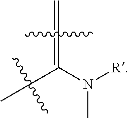

113. The compound of embodiment 110 or 112, wherein one R′ and another R group on another atom are taken together with their intervening atoms to form an optionally substituted 3-10 membered ring having, in addition to the intervening atoms, 0-4 heteroatoms.

114. The compound of embodiment 113, wherein one R′ and R³ on another atom are taken together with their intervening atoms to form an optionally substituted 3-10 membered ring having, in addition to the intervening atoms, 0-4 heteroatoms.

115. The compound of any one of embodiments 1 and 63-79, wherein the compound has a structure of

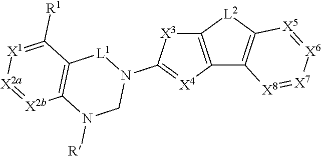

or a salt thereof.

116. The compound of any one of embodiments 1 and 63-79, wherein the compound has a structure of

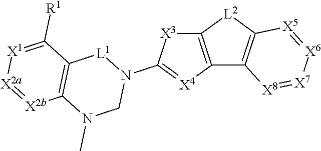

or a salt thereof.

117. The compound of any one of embodiments 1-4, 17, 46 and 63-88, wherein X^{2c} is —C(halogen)=.

118. The compound of any one of embodiments 1-4, 17, 46 and 63-88, wherein X^{2c} is —C(Cl)═.

119. The compound of any one of embodiments 1-4, 17, 46 and 63-88, wherein X^{2c} is —C(Br)═.

120. The compound of any one of embodiments 1-4, 17, 46 and 63-119, wherein X^{1a} is —N(R′)—.

121. The compound of any one of embodiments 1-4, 46 and 63-119, wherein X^{1a} is —N(R′)— wherein R′ is optionally substituted C₁-C₆ aliphatic.

122. The compound of any one of embodiments 1-4, 46 and 63-119, wherein X^{1a} is —N(R′)— wherein R′ is C₁-C₆ aliphatic.

123. The compound of any one of embodiments 1-4, 46 and 63-119, wherein X^{1a} is —N(R′)— wherein R′ is optionally substituted C₁-C₆ alkyl.

124. The compound of any one of embodiments 1-4, 46 and 63-119, wherein X^{1a} is —N(Me)-.

125. The compound of any one of embodiments 1-4, 46 and 63-119, wherein X^{1a} is —O—.

126. The compound of any one of embodiments 1-4, 46 and 63-119, wherein X^{1a} is —S—.

127. The compound of any one of embodiments 1-4, 46 and 63-119, wherein X^{1a} is optionally substituted —CH₂—.

128. The compound of any one of embodiments 1-4, 46 and 63-119, wherein X^{1a} is —C(R′)₂-129. The compound of any one of embodiments 1-4, 46 and 63-119, wherein X^{1a} is optionally substituted —NH—.

130. The compound of any one of embodiments 1 and 5-129, wherein X³ is —S—.

131. The compound of any one of embodiments 1 and 5-129, wherein X³ is —Se—.

132. The compound of any one of embodiments 1 and 5-129, wherein X³ is —O—.

133. The compound of any one of embodiments 1 and 5-129, wherein X³ is —N(R′)—.

134. The compound of any one of embodiments 1 and 5-129, wherein X³ is —N(R′)— wherein R′ is optionally substituted C₁-C₆ aliphatic.

135. The compound of any one of embodiments 1 and 5-129, wherein X³ is —N(R′)— wherein R′ is C₁-C₆ aliphatic.

136. The compound of any one of embodiments 1 and 5-129, wherein X³ is —N(R′)— wherein R′ is optionally substituted C₁-C₆ alkyl.

137. The compound of any one of embodiments 1 and 5-129, wherein X³ is optionally substituted —NH—.

138. The compound of any one of embodiments 1 and 5-129, wherein X³ is —N(Me)-.

139. The compound of any one of embodiments 1 and 5-138, wherein X⁴ is —N═.

140. The compound of any one of embodiments 1 and 5-138, wherein X⁴ is optionally substituted —CH═.

141. The compound of any one of embodiments 1 and 5-138, wherein X⁴ is —C(R⁶)═.

142. The compound of any one of embodiments 1 and 5-141, wherein X⁵ is —N═.

143. The compound of any one of embodiments 1 and 5-141, wherein X⁵ is optionally substituted —CH═.

144. The compound of any one of embodiments 1 and 5-141, wherein X⁵ is —CH═.

145. The compound of any one of embodiments 1 and 5-141, wherein X⁵ is —C(R⁶)═.

146. The compound of any one of embodiments 1 and 5-141, wherein X⁵ is —C(R⁶)═ wherein R⁶ is optionally substituted C₁-C₆ aliphatic.

147. The compound of any one of embodiments 1 and 5-141, wherein X⁵ is —C(R⁶)═ wherein R⁶ is C₁-C₆ aliphatic.

148. The compound of any one of embodiments 1 and 5-141, wherein X⁵ is —C(R⁶)═ wherein R⁶ is optionally substituted C₁-C₆ alkyl.

149. The compound of any one of embodiments 1 and 5-141, wherein X⁵ is —C(CF₃)═.

150. The compound of any one of embodiments 1 and 5-149, wherein X⁶ is —N═.

151. The compound of any one of embodiments 1 and 5-149, wherein X⁶ is optionally substituted —CH═.

152. The compound of any one of embodiments 1 and 5-149, wherein X⁶ is —CH═.

153. The compound of any one of embodiments 1 and 5-149, wherein X⁶ is —C(R⁶)═.

154. The compound of any one of embodiments 1 and 5-149, wherein X⁶ is —C(OR′)═.

155. The compound of any one of embodiments 1 and 5-149, wherein X⁶ is —C(OH)═.

156. The compound of any one of embodiments 1 and 5-149, wherein X⁶ is —C(OR′)═ wherein R′ is optionally substituted C₁-C₆ aliphatic.

157. The compound of any one of embodiments 1 and 5-149, wherein X⁶ is —C(OR′)═ wherein R′ is C₁-C₆ aliphatic.

158. The compound of any one of embodiments 1 and 5-149, wherein X⁶ is —C(OR′)═ wherein R′ is optionally substituted C₁-C₆ alkyl.

159. The compound of any one of embodiments 1 and 5-149, wherein X⁶ is —C(OR′)═ wherein R′ is C₁-C₆ alkyl.

160. The compound of any one of embodiments 1 and 5-149, wherein X⁶ is —C(OR′)═ wherein R′ is substituted C₁-C₆ alkyl and each substituent is —F.

161. The compound of any one of embodiments 1 and 5-149, wherein X⁶ is —C(OMe)═.

162. The compound of any one of embodiments 1 and 5-149, wherein X⁶ is —C(OCF₃)═.

163. The compound of any one of embodiments 1 and 5-149, wherein X⁶ is —C(OCHF₂)═.

164. The compound of any one of embodiments 1 and 5-149, wherein X⁶ is —C(OCH₂F)═.

165. The compound of any one of embodiments 1 and 5-149, wherein X⁶ is —C(OCF₂Cl)═.

166. The compound of any one of embodiments 1 and 5-149, wherein X⁶ is —C(OR′)═ wherein R′ is taken together with another R′ group on another atom and their intervening atoms to form an optionally substituted 3-10 membered ring having, in addition to the intervening atoms, 0-4 heteroatoms.

167. The compound of any one of embodiments 1-149, wherein the compound has a structure of

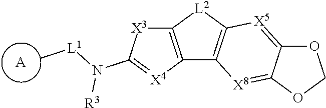

or a salt thereof.

168. The compound of any one of embodiments 1 and 5-149, wherein X⁶ is —C(OR′)═ wherein R′ is —C(O)R.

169. The compound of any one of embodiments 1 and 5-149, wherein X⁶ is —C(OR′)═ wherein R′ is —C(O)R and R is optionally substituted C₁-C₆ aliphatic.

170. The compound of any one of embodiments 1 and 5-149, wherein X⁶ is

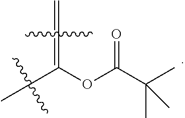 [SMILES: *C(=*)OC(=O)C(C)(C)C]

171. The compound of any one of embodiments 1 and 5-149, wherein X⁶ is —C(R⁶)═ wherein R⁶ is —C(O)OR′.

172. The compound of any one of embodiments 1 and 5-149, wherein X⁶ is —C(R⁶)═ wherein R⁶ is —C(O)OR′ and R′ is optionally substituted C₁-C₆ aliphatic.

173. The compound of any one of embodiments 1 and 5-149, wherein X⁶ is —C(R⁶)═ wherein R⁶ is —C(O)OCH₃.

174. The compound of any one of embodiments 1 and 5-149, wherein X⁶ is —C(R⁶)═ wherein R⁶ is —C(O)OCH₂CH₃.

175. The compound of any one of embodiments 1 and 5-149, wherein X⁶ is —C(R⁶)═ wherein R⁶ is —C(O)R.

176. The compound of any one of embodiments 1 and 5-149, wherein X⁶ is —C(R⁶)═ wherein R⁶ is —C(O)R and R is optionally substituted C₁-C₆ aliphatic.

177. The compound of any one of embodiments 1 and 5-149, wherein X⁶ is —C(R⁶)═ wherein R⁶ is —C(O)CH₃.

178. The compound of any one of embodiments 1 and 5-149, wherein X⁶ is —C(R⁶)═ wherein R⁶ is —CN.

179. The compound of any one of embodiments 1 and 5-149, wherein X⁶ is —C(R⁶)═ wherein R⁶ is —NO₂.

180. The compound of any one of embodiments 1 and 5-149, wherein X⁶ is —C(R⁶)═ wherein R⁶ is —N(R′)₂.

181. The compound of any one of embodiments 1 and 5-149, wherein X⁶ is —C(R⁶)═ wherein R⁶ is —N(R′)₂ and each R′ is independently optionally substituted C₁-C₆ aliphatic.

182. The compound of any one of embodiments 1 and 5-149, wherein X⁶ is —C(R⁶)═ wherein R⁶ is —N(R′)₂ and each R′ is independently C₁-C₆ aliphatic.

183. The compound of any one of embodiments 1 and 5-149, wherein X⁶ is —C(R⁶)═ wherein R⁶ is —N(R′)₂ and each R′ is independently optionally substituted C₁-C₆ alkyl.

184. The compound of any one of embodiments 1 and 5-149, wherein X⁶ is —C(R⁶)═ wherein R⁶ is —N(R′)₂ and two R′ are taken together with the intervening nitrogen atom to form an optionally substituted 3-10 membered ring having, in addition to the nitrogen atom, 0-4 heteroatoms.

185. The compound of any one of embodiments 1 and 5-149, wherein X⁶ is —C(R⁶)═ wherein R⁶ is —N(R′)₂ and two R′ are taken together with the intervening nitrogen atom to form an optionally substituted 3-6 membered ring having, in addition to the nitrogen atom, 0 heteroatoms.

186. The compound of any one of embodiments 1 and 5-149, wherein X⁶ is —C(R⁶)═ wherein R⁶ is —N(R′)₂ and two R′ are taken together with the intervening nitrogen atom to form an optionally substituted 3-6 membered ring having, in addition to the nitrogen atom, 1 heteroatom.

187. The compound of any one of embodiments 1 and 5-149, wherein X⁶ is —C(R⁶)═ wherein R⁶ is —N(R′)₂ and two R′ are taken together with the intervening nitrogen atom to form an optionally substituted 3-6 membered ring having, in addition to the nitrogen atom, 2 heteroatoms.

188. The compound of any one of embodiments 1 and 5-149, wherein X⁶ is —C(R⁶)═ wherein R⁶ is

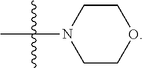 [SMILES: *N1CCOCC1]

189. The compound of any one of embodiments 1 and 5-149, wherein X⁶ is —C(R⁶)═ wherein R⁶ is optionally substituted C₁-C₁₀ aliphatic.

190. The compound of any one of embodiments 1 and 5-149, wherein X⁶ is —C(R⁶)═ wherein R⁶ is optionally substituted C₁-C₆ aliphatic.

191. The compound of any one of embodiments 1 and 5-149, wherein X⁶ is —C(R⁶)═ wherein R⁶ is C₁-C₆ aliphatic.

192. The compound of any one of embodiments 1 and 5-149, wherein X⁶ is —C(R⁶)═ wherein R⁶ is optionally substituted C₁-C₆ alkyl.

193. The compound of any one of embodiments 1 and 5-149, wherein X⁶ is —C(R⁶)═ wherein R⁶ is C₁-C₆ haloalkyl.

194. The compound of any one of embodiments 1 and 5-149, wherein X⁶ is —C(R⁶)═ wherein R⁶ is substituted C₁-C₆ alkyl and each substituent is —F.

195. The compound of any one of embodiments 1 and 5-149, wherein X⁶ is —C(R⁶)═ wherein R⁶ is C₁-C₆ alkyl.

196. The compound of any one of embodiments 1 and 5-149, wherein X⁶ is —C(R⁶)═ wherein R⁶ is optionally substituted C₂-C₆ alkenyl.

197. The compound of any one of embodiments 1 and 5-149, wherein X⁶ is —C(R⁶)═ wherein R⁶ is C₂—C₆ alkenyl.

198. The compound of any one of embodiments 1 and 5-149, wherein X⁶ is —C(R⁶)═ wherein R⁶ is optionally substituted C₂-C₆ alkynyl.

199. The compound of any one of embodiments 1 and 5-149, wherein X⁶ is —C(R⁶)═ wherein R⁶ is C₂-C₆ alkynyl.

200. The compound of any one of embodiments 1 and 5-149, wherein X⁶ is —C(R⁶)═ wherein R⁶ is methyl.

201. The compound of any one of embodiments 1 and 5-149, wherein X⁶ is —C(R⁶)═ wherein R⁶ is ethyl.

202. The compound of any one of embodiments 1 and 5-149, wherein X⁶ is —C(R⁶)═ wherein R⁶ is —CF₃.

203. The compound of any one of embodiments 1 and 5-149, wherein X⁶ is —C(R⁶)═ wherein R⁶ is —CH₂F.

204. The compound of any one of embodiments 1 and 5-149, wherein X⁶ is —C(R⁶)═ wherein R⁶ is —CHF₂.

205. The compound of any one of embodiments 1 and 5-149, wherein X⁶ is —C(R⁶)═ wherein R⁶ is —CF₂Cl.

206. The compound of any one of embodiments 1 and 5-149, wherein X⁶ is —C(R⁶)═ wherein R⁶ is —CH(CH₃)₂.

207. The compound of any one of embodiments 1 and 5-149, wherein X⁶ is —C(R⁶)═ wherein R⁶ is —C(CH₃)₂OH.

208. The compound of any one of embodiments 1 and 5-149, wherein X⁶ is —C(R⁶)═ wherein R⁶ is —C(CH₃)₂CN.

209. The compound of any one of embodiments 1 and 5-149, wherein X⁶ is —C(R⁶)═ wherein R⁶ is —C(CH₃)═CH₂.

210. The compound of any one of embodiments 1 and 5-149, wherein X⁶ is —C(R⁶)═ wherein R⁶ is —C ═CH.

211. The compound of any one of embodiments 1 and 5-149, wherein X⁶ is —C(R⁶)═ wherein R⁶ is optionally substituted 3-10 membered cycloaliphatic.

212. The compound of any one of embodiments 1 and 5-149, wherein X⁶ is —C(R⁶)═ wherein R⁶ is optionally substituted 3-6 membered cycloaliphatic.

213. The compound of any one of embodiments 1 and 5-149, wherein X⁶ is —C(R⁶)═ wherein R⁶ is 3-6 membered cycloaliphatic.

214. The compound of any one of embodiments 1 and 5-149, wherein X⁶ is —C(R⁶)═ wherein R⁶ is cyclopropyl.

215. The compound of any one of embodiments 1 and 5-149, wherein X⁶ is —C(R⁶)═ wherein R⁶ is cyclobutyl.

216. The compound of any one of embodiments 1 and 5-149, wherein X⁶ is —C(R⁶)═ wherein R⁶ is cyclopentyl.

217. The compound of any one of embodiments 1 and 5-149, wherein X⁶ is —C(R⁶)═ wherein R⁶ is cyclohexyl.

218. The compound of any one of embodiments 1 and 5-149, wherein X⁶ is —C(R⁶)═ wherein R⁶ is halogen.

219. The compound of any one of embodiments 1 and 5-149, wherein X⁶ is —C(F)═.

220. The compound of any one of embodiments 1 and 5-149, wherein X⁶ is —C(Cl)═.

221. The compound of any one of embodiments 1 and 5-149, wherein X⁶ is —C(Br)═.

222. The compound of any one of embodiments 1 and 5-149, wherein X⁶ is —C(I)═.

223. The compound of any one of embodiments 1 and 5-222, wherein X⁷ is —N═.

224. The compound of any one of embodiments 1 and 5-222, wherein X⁷ is optionally substituted —CH═.

225. The compound of any one of embodiments 1 and 5-222, wherein X⁷ is —C(R⁶)═.

226. The compound of any one of embodiments 1 and 5-222, wherein X⁷ is —CH═.

227. The compound of any one of embodiments 1 and 5-222, wherein X⁷ is —C(OH)═.

228. The compound of any one of embodiments 1 and 5-222, wherein X⁷ is —C(OR′)═ wherein R′ is optionally substituted C₁-C₆ aliphatic.

229. The compound of any one of embodiments 1 and 5-222, wherein X⁷ is —C(OR′)═ wherein R′ is C₁-C₆ aliphatic.

230. The compound of any one of embodiments 1 and 5-222, wherein X⁷ is —C(OR′)═ wherein R′ is optionally substituted C₁-C₆ alkyl.

231. The compound of any one of embodiments 1 and 5-222, wherein X⁷ is —C(OR′)═ wherein R′ is C₁-C₆ haloalkyl.

232. The compound of any one of embodiments 1 and 5-222, wherein X⁷ is —C(OR′)═ wherein R′ is substituted C₁-C₆ alkyl and each substituent is —F.

233. The compound of any one of embodiments 1 and 5-222, wherein X⁷ is —C(OMe)═.

234. The compound of any one of embodiments 1 and 5-222, wherein X⁷ is —C(OCF₃)═.

235. The compound of any one of embodiments 1 and 5-222, wherein X⁷ is —C(OCHF₂)═.

236. The compound of any one of embodiments 1 and 5-222, wherein X⁷ is —C(OCH₂F)═.

237. The compound of any one of embodiments 1 and 5-222, wherein X⁷ is —C(OCF₂Cl)═.

238. The compound of any one of embodiments 1 and 5-222, wherein X⁷ is —C(OR′)═ wherein R′ is taken together with another R′ group on another atom and their intervening atoms to form an optionally substituted 3-10 membered ring having, in addition to the intervening atoms, 0-4 heteroatoms.

239. The compound of any one of embodiments 1 and 5-222, wherein X⁷ is —C(R⁶)═ wherein R⁶ is optionally substituted C₁-C₁₀ aliphatic.

240. The compound of any one of embodiments 1 and 5-222, wherein X⁷ is —C(R⁶)═ wherein R⁶ is optionally substituted C₁-C₆ aliphatic.

241. The compound of any one of embodiments 1 and 5-222, wherein X⁷ is —C(R⁶)═ wherein R⁶ is C₁-C₆ aliphatic.

242. The compound of any one of embodiments 1 and 5-222, wherein X⁷ is —C(R⁶)═ wherein R⁶ is optionally substituted C₁-C₆ alkyl.

243. The compound of any one of embodiments 1 and 5-222, wherein X⁷—C(R⁶)═ wherein R⁶ is halogen substituted C₁-C₆ alkyl.

244. The compound of any one of embodiments 1 and 5-222, wherein X⁷ is —C(R⁶)═ wherein R⁶ is substituted C₁-C₆ alkyl and each substituent is —F.

245. The compound of any one of embodiments 1 and 5-222, wherein X⁷ is —C(R⁶)═ wherein R⁶ s C₁-C₆ alkyl.

246. The compound of any one of embodiments 1 and 5-222, wherein X⁷ is —C(R⁶)═ wherein R⁶ is optionally substituted C₂-C₆ alkenyl.

247. The compound of any one of embodiments 1 and 5-222, wherein X⁷ is —C(R⁶)═ wherein R⁶ is C₂-C₆ alkenyl.

248. The compound of any one of embodiments 1 and 5-222, wherein X⁷ is —C(R⁶)═ wherein R⁶ is optionally substituted C₂-C₆ alkynyl.

249. The compound of any one of embodiments 1 and 5-222, wherein X⁷ is —C(R⁶)═ wherein R⁶ is C₂-C₆ alkynyl.

250. The compound of any one of embodiments 1 and 5-222, wherein X⁷ is —C(R⁶)═ wherein R⁶ is methyl.

251. The compound of any one of embodiments 1 and 5-222, wherein X⁷ is —C(R⁶)═ wherein R⁶ is ethyl. 252. The compound of any one of embodiments 1 and 5-222, wherein X⁷ is —C(R⁶)═ wherein R⁶ is —CF₃.

253. The compound of any one of embodiments 1 and 5-222, wherein X⁷ is —C(R⁶)═ wherein R⁶ is —CH₂F.

254. The compound of any one of embodiments 1 and 5-222, wherein X⁷ is —C(R⁶)═ wherein R⁶ is —CHF₂.

255. The compound of any one of embodiments 1 and 5-222, wherein X⁷ is —C(R⁶)═ wherein R⁶ is —CF₂Cl.

256. The compound of any one of embodiments 1 and 5-222, wherein X⁷ is —C(R⁶)═ wherein R⁶ is halogen.

257. The compound of any one of embodiments 1 and 5-222, wherein X⁷ is —C(F)═.

258. The compound of any one of embodiments 1 and 5-222, wherein X⁷ is —C(Cl)═.

259. The compound of any one of embodiments 1 and 5-222, wherein X⁷ is —C(Br)═.

260. The compound of any one of embodiments 1 and 5-222, wherein X⁷ is —C(I)═.

261. The compound of any one of embodiments 1 and 5-260, wherein X⁸ is —N═.

262. The compound of any one of embodiments 1 and 5-260, wherein X⁸ is optionally substituted —CH═.

263. The compound of any one of embodiments 1 and 5-260, wherein X⁸ is —C(R⁶)═.

264. The compound of any one of embodiments 1 and 5-260, wherein X⁸ is —CH═.

265. The compound of any one of embodiments 1 and 5-260, wherein X⁸ is —C(OH)—.

266. The compound of any one of embodiments 1 and 5-260, wherein X⁸ is —C(OR′)═ wherein R′ is optionally substituted C₁-C₆ aliphatic.

267. The compound of any one of embodiments 1 and 5-260, wherein X⁸ is —C(OR′)═ wherein R′ is C₁-C₆ aliphatic.

268. The compound of any one of embodiments 1 and 5-260, wherein X⁸ is —C(OR′)═ wherein R′ is optionally substituted C₁-C₆ alkyl.

269. The compound of any one of embodiments 1 and 5-260, wherein X⁸ is —C(OR′)═ wherein R′ is halogen substituted C₁-C₆ alkyl.

270. The compound of any one of embodiments 1 and 5-260, wherein X⁸ is —C(OR′)═ wherein R′ is substituted C₁-C₆ alkyl and each substituent is —F.

271. The compound of any one of embodiments 1 and 5-260, wherein X⁸ is —C(OMe)═.

272. The compound of any one of embodiments 1 and 5-260, wherein X⁸ is —C(OCF₃)═.

273. The compound of any one of embodiments 1 and 5-260, wherein X⁸ is —C(OCHF₂)═.

274. The compound of any one of embodiments 1 and 5-260, wherein X⁸ is —C(OCH₂F)═.

275. The compound of any one of embodiments 1 and 5-260, wherein X⁸ is —C(OCF₂Cl)═.

276. The compound of any one of embodiments 1 and 5-260, wherein X⁸ is —C(OR′)═ wherein R′ is taken together with another R′ group on another atom and their intervening atoms to form an optionally substituted 3-10 membered ring having, in addition to the intervening atoms, 0-4 heteroatoms.

277. The compound of any one of embodiments 1 and 5-260, wherein the compound has a structure of

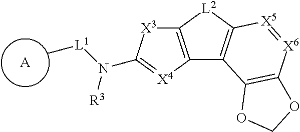

or a salt thereof.

278. The compound of any one of embodiments 1 and 5-260, wherein X⁸ is —C(R⁶)═ wherein R⁶ is optionally substituted C₁-C₁₀ aliphatic.

279. The compound of any one of embodiments 1 and 5-260, wherein X⁸ is —C(R⁶)═ wherein R⁶ is optionally substituted C₁-C₆ aliphatic.

280. The compound of any one of embodiments 1 and 5-260, wherein X⁸ is —C(R⁶)═ wherein R⁶ is C₁-C₆ aliphatic.

281. The compound of any one of embodiments 1 and 5-260, wherein X⁸ is —C(R⁶)═ wherein R⁶ is optionally substituted C₁-C₆ alkyl.

282. The compound of any one of embodiments 1 and 5-260, wherein X⁸ is —C(R⁶)═ wherein R⁶ is halogen substituted C₁-C₆ alkyl.

283. The compound of any one of embodiments 1 and 5-260, wherein X⁸ is —C(R⁶)═ wherein R⁶ is substituted C₁-C₆ alkyl and each substituent is —F.

284. The compound of any one of embodiments 1 and 5-260, wherein X⁸ is —C(R⁶)═ wherein R⁶ is C₁-C₆ alkyl.

285. The compound of any one of embodiments 1 and 5-260, wherein X⁸ is —C(R⁶)═ wherein R⁶ is optionally substituted C₂-C₆ alkenyl.

286. The compound of any one of embodiments 1 and 5-260, wherein X⁸ is —C(R⁶)═ wherein R⁶ is C₂-C₆ alkenyl.

287. The compound of any one of embodiments 1 and 5-260, wherein X⁸ is —C(R⁶)═ wherein R⁶ is optionally substituted C₂-C₆ alkynyl.

288. The compound of any one of embodiments 1 and 5-260, wherein X⁸ is —C(R⁶)═ wherein R⁶ is C₂-C₆ alkynyl.

289. The compound of any one of embodiments 1 and 5-260, wherein X⁸ is —C(R⁶)═ wherein R⁶ is methyl.

290. The compound of any one of embodiments 1 and 5-260, wherein X⁸ is —C(R⁶)═ wherein R⁶ is ethyl.

291. The compound of any one of embodiments 1 and 5-260, wherein X⁸ is —C(R⁶)═ wherein R⁶ is —CF₃.

292. The compound of any one of embodiments 1 and 5-260, wherein X⁸ is —C(R⁶)═ wherein R⁶ is —CH₂F.

293. The compound of any one of embodiments 1 and 5-260, wherein X⁸ is —C(R⁶)═ wherein R⁶ is CHF₂.

294. The compound of any one of embodiments 1 and 5-260, wherein X⁸ is —C(R⁶)═ wherein R⁶ is —CF₂Cl.

295. The compound of any one of embodiments 1 and 5-260, wherein X⁸ is —C(R⁶)═ wherein R⁶ is halogen.

296. The compound of any one of embodiments 1 and 5-260, wherein X⁸ is —C(F)═.

297. The compound of any one of embodiments 1 and 5-260, wherein X⁸ is —C(Cl)═.

298. The compound of any one of embodiments 1 and 5-260, wherein X⁸ is —C(Br)═.

299. The compound of any one of embodiments 1 and 5-260, wherein X⁸ is —C(I)═.

300. The compound of any one of embodiments 1~4 and 17-299, wherein R¹ is —OR′.

301. The compound of any one of embodiments 1~4 and 17-299, wherein R¹ is —OH.

302. The compound of embodiment 300, wherein R′ is —OR′ wherein R′ is optionally substituted C₁-C₆ aliphatic.

303. The compound of embodiment 300, wherein R¹ is —OR′ wherein R′ is C₁-C₆ aliphatic.

304. The compound of embodiment 300, wherein R¹ is —OR′ wherein R′ is optionally substituted C₁-C₆ alkyl.

305. The compound of embodiment 300, wherein R¹ is —OR′ wherein R′ is C₁-C₆ alkyl.

306. The compound of embodiment 300, wherein R¹ is —OR′ wherein R′ is halogen substituted C₁-C₆ alkyl.

307. The compound of embodiment 300, wherein R¹ is —OR′ wherein R′ is substituted C₁-C₆ alkyl and each substituent is —F.

308. The compound of embodiment 300, wherein R¹ is —OMe.

309. The compound of embodiment 300, wherein R¹ is —OCF₃.

310. The compound of embodiment 300, wherein R¹ is —OCHF₂.

311. The compound of embodiment 300, wherein R¹ is —OCH₂F.

312. The compound of embodiment 300, wherein R¹ is —OCF₂Cl.

313. The compound of embodiment 300, wherein R¹ is —OCH₂CF₃.

314. The compound of any one of embodiments 1~4 and 17-299, wherein R¹ is optionally substituted C₁-C₁₀ aliphatic.

315. The compound of embodiment 314, wherein R¹ is optionally substituted C₁-C₆ aliphatic.

316. The compound of embodiment 314, wherein R¹ is C₁-C₆ aliphatic.

317. The compound of embodiment 314, wherein R¹ is optionally substituted C₁-C₆ alkyl.

318. The compound of embodiment 314, wherein R¹ is halogen substituted C₁-C₆ alkyl.

319. The compound of embodiment 314, wherein R¹ is substituted C₁-C₆ alkyl and each substituent is —F.

320. The compound of embodiment 314, wherein R¹ is C₁-C₆ alkyl.

321. The compound of embodiment 314, wherein R¹ is optionally substituted C₂-C₆ alkenyl.

322. The compound of embodiment 314, wherein R¹ is C₂-C₆ alkenyl.

323. The compound of embodiment 314, wherein R¹ is optionally substituted C₂-C₆ alkynyl.

324. The compound of embodiment 314, wherein R¹ is C₂-C₆ alkynyl.

325. The compound of embodiment 314, wherein R¹ is methyl.

326. The compound of embodiment 314, wherein R¹ is ethyl.

327. The compound of embodiment 314, wherein R¹ is —CF₃.

328. The compound of embodiment 314, wherein R′ is —CH₂F.

329. The compound of embodiment 314, wherein R¹ is —CHF₂.

330. The compound of embodiment 314, wherein R¹ is —CF₂Cl.

331. The compound of any one of embodiments 1~4 and 17-299, wherein R¹ is halogen.

332. The compound of embodiment 331, wherein R¹ is —F.

333. The compound of embodiment 331, wherein R¹ is —Cl.

334. The compound of embodiment 331, wherein R¹ is —Br.

335. The compound of embodiment 331, wherein R¹ is —I.

336. The compound of any one of embodiments 1 and 4-335, wherein L¹ is covalent bond.

337. The compound of any one of embodiments 1 and 4-335, wherein L¹ is —C(O)—.

338. The compound of any one of embodiments 1 and 4-335, wherein L¹ is —C(S)—.

339. The compound of any one of embodiments 1 and 4-335, wherein L¹ is —S(O)₂—.

340. The compound of any one of embodiments 1 and 4-335, wherein L¹ is optionally substituted —CH₂—.

341. The compound of any one of embodiments 1 and 4-335, wherein L¹ is —CH₂—.

342. The compound of any one of embodiments 1 and 4-335, wherein L¹ is —N(R′)C(O)—.

343. The compound of any one of embodiments 1 and 4-335, wherein L¹ is —N(R′)C(O)— wherein R′ is C₁-C₆ alkyl.

344. The compound of any one of embodiments 1 and 4-335, wherein L¹ is —N(H) C(O)—.

345. The compound of any one of embodiments 1 and 4-335, wherein L¹ is —N(R′)S(O)₂—.

346. The compound of any one of embodiments 1 and 4-335, wherein L¹ is —N(R′)S(O)₂— wherein R′ is C₁-C₆ alkyl.

347. The compound of any one of embodiments 1 and 4-335, wherein L¹ is —N(H)S(O)₂—.

348. The compound of any one of embodiments 1 and 4-335, wherein L¹ is —N(R′)C(S)—.

349. The compound of any one of embodiments 1 and 4-335, wherein L¹ is —N(R′)C(S)— wherein R′ is C₁-C₆ alkyl.

350. The compound of any one of embodiments 1 and 4-335, wherein L¹ is —N(H) C(S)—.

351. The compound of any one of embodiments 1 and 4-335, wherein L¹ is

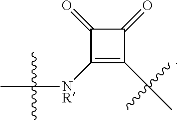

352. The compound of any one of embodiments 1 and 5-351, wherein R³ is H.

353. The compound of any one of embodiments 1 and 5-351, wherein R³ is optionally substituted C₁-C₆ aliphatic.

354. The compound of embodiment 353, wherein R³ is optionally substituted C₁-C₆ alkyl.

355. The compound of embodiment 353, wherein R³ is C₁-C₆ alkyl.

356 The compound of embodiment 353, wherein R³ is methyl.

357. The compound of embodiment 353, wherein R³ is ethyl.

358. The compound of any one of embodiments 1 and 5-357, wherein L² is optionally substituted C₁₋₃ alkylene.

359. The compound of embodiment 358, wherein L² is C₁₋₃ alkylene.

360. The compound of embodiment 358, wherein L² is optionally substituted —CH₂—.

361. The compound of embodiment 358, wherein L² is optionally substituted —CH₂CH₂—.

362. The compound of embodiment 358, wherein L² is optionally substituted —CH₂CH₂CH₂—.

363. The compound of embodiment 358, wherein L² is —CH₂—.

364. The compound of embodiment 358, wherein L² is —CH₂CH₂—.

365. The compound of embodiment 358, wherein L² is —CH₂CH₂CH₂—.

366. The compound of any one of embodiments 1 and 5-357, wherein L² is optionally substituted C₁₋₃ alkylene, wherein one or more methylene units of the alkylene are independently replaced with —O—.

367. The compound of any one of embodiments 1 and 5-357, wherein L² is optionally substituted C₁₋₃ alkylene, wherein one unit of the alkylene is replaced with —O—.

368. The compound of embodiment 366, wherein L² is optionally substituted —CH₂O—.

369 The compound of embodiment 366, wherein L² is —CH₂O—.

370. The compound of embodiment 366, wherein L² is substituted —CH₂O—.

371. The compound of embodiment 366, wherein L² is optionally substituted —CH₂CH₂O—.

372. The compound of embodiment 366, wherein L² is —CH₂CH₂O—.

373. The compound of embodiment 366, wherein L² is substituted —CH₂CH₂O—.

374. The compound of any one of embodiments 1 and 5-357, wherein L² is an optionally substituted C₂₋₃ alkylene, wherein one unit of the alkylene is replaced with —O— and another unit of the alkylene is replaced with —C(R⁴)(R⁵)—.

375. The compound of any one of embodiments 1 and 5-357, wherein L² is

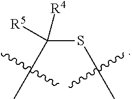

376. The compound of any one of embodiments 1 and 5-357, wherein L² is optionally substituted C₁₋₃ alkylene, wherein one or more methylene units of the alkylene are independently replaced with —C(R⁴)(R⁵)—.

377. The compound of any one of embodiments 1 and 5-357, wherein L² is optionally substituted C₁₋₃ alkylene, wherein one methylene unit of the alkylene is replaced with —C(R⁴)(R⁵)—.

378. The compound of any one of embodiments 1 and 5-357, wherein L² is

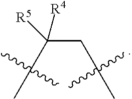

379. The compound of any one of embodiments 1 and 5-357, wherein L² is optionally substituted C₁₋₃ alkylene, wherein one or more methylene units of the alkylene are independently replaced with —N(R⁴)—.

380. The compound of any one of embodiments 1 and 5-357, wherein L² is n optionally substituted C₁₋₃ alkylene, wherein one methylene unit of the alkylene is replaced with —N(R⁴)—.

381. The compound of any one of embodiments 1 and 5-357, wherein L² is —CH₂—N(R⁴)—, wherein the —CH₂— is optionally substituted.

382. The compound of any one of embodiments 1 and 5-357, wherein L² is —CH₂—N(R⁴)—, wherein the —CH₂— is substituted.

383. The compound of any one of embodiments 1 and 5-357, wherein L² is —CH₂—N(R⁴)—, wherein the —CH₂— is unsubstituted.

384. The compound of any one of embodiments 1 and 5-357, wherein L² is —C(R⁴)(R⁵)—N(R⁴)—.

385. The compound of any one of embodiments 1 and 5-357, wherein L² is —C(R⁴)(R⁵)—NH—.

386. The compound of embodiment 374, wherein L² is —C(R⁴)(R⁵)—O—.

387. The compound of any one of embodiments 1 and 5-386, wherein R⁴ is R.

388. The compound of embodiment 387, wherein R⁴ is —H.

389. The compound of embodiment 387, wherein R⁴ is not —H.

390. The compound of embodiment 387, wherein R⁴ is optionally substituted C₁-C₁₀ aliphatic.

391. The compound of embodiment 387, wherein R⁴ is optionally substituted C₁₋₆ aliphatic.

392. The compound of embodiment 387, wherein R⁴ is optionally substituted C₁₋₆ alkyl.

393. The compound of embodiment 387, wherein R⁴ is C₁₋₆ alkyl.

394. The compound of embodiment 387, wherein R⁴ is methyl.

395. The compound of embodiment 387, wherein R⁴ is —CH₂F.

396. The compound of embodiment 387, wherein R⁴ is ethyl.

397. The compound of embodiment 387, wherein R⁴ is —CH₂CH₂CH═CH₂.

398. The compound of embodiment 387, wherein R⁴ is optionally substituted phenyl.

399. The compound of embodiment 387, wherein R⁴ and R⁵ are taken together with the atom to which they are attached to form an optionally substituted 3-10 membered ring having 0-4 heteroatoms.

400. The compound of embodiment 387, wherein R⁴ and R⁵ are taken together with the atom to which they are attached to form an optionally substituted 3-10, e.g., 3, 4, 5, 6, 7, 8, 9, or 10, membered ring having no heteroatoms.

401. The compound of embodiment 387, wherein R⁴ and R⁵ are taken together with the atom to which they are attached to form an optionally substituted 3-membered ring having no heteroatoms.

402. The compound of embodiment 387, wherein R⁴ and R⁵ are taken together with the atom to which they are attached to form an optionally substituted 4-membered ring having no heteroatoms.

403. The compound of embodiment 387, wherein R⁴ and R⁵ are taken together with the atom to which they are attached to form an optionally substituted 6-membered ring having no heteroatoms.

404. The compound of embodiment 387, wherein R⁴ and R⁵ are taken together with the atom to which they are attached to form an optionally substituted cyclohexyl ring.

405. The compound of embodiment 387, wherein R⁴ and R⁵ are taken together with the atom to which they are attached to form an optionally substituted 3-10 membered ring having 1-4 heteroatoms.

406. The compound of embodiment 387, wherein R⁴ and R⁵ are taken together with the atom to which they are attached to form an optionally substituted 3-10 membered ring one heteroatom.

407. The compound of embodiment 387, wherein R⁴ and R⁵ are taken together with the atom to which they are attached to form an optionally substituted 3-membered ring one heteroatom.

408. The compound of embodiment 387, wherein R⁴ and R⁵ are taken together with the atom to which they are attached to form an optionally substituted 4-membered ring one heteroatom.

409. The compound of embodiment 387, wherein R⁴ and R⁵ are taken together with the atom to which they are attached to form an optionally substituted 6-membered ring one heteroatom.

410. The compound of any one of embodiments 405-409, wherein the formed ring has a nitrogen atom.

411. The compound of any one of embodiments 405-409, wherein the formed ring has an oxygen atom.

412. The compound of any one of embodiments 1 and 5-398, wherein R⁵ is R.

413. The compound of embodiment 412, wherein R⁵ is —H.

414. The compound of embodiment 412, wherein R⁵ is not —H.

415. The compound of embodiment 412, wherein R⁵ is optionally substituted C₁-C₁₀ aliphatic.

416. The compound of embodiment 412, wherein R⁵ is optionally substituted C₁₋₆ aliphatic.

417. The compound of embodiment 412, wherein R⁵ is methyl.

418. The compound of embodiment 412, wherein R⁵ is —CH₂F.

419 The compound of embodiment 412, wherein R⁵ is ethyl.

420. The compound of embodiment 412, wherein R⁵ is —CH₂CH₂CH═CH₂.

421. The compound of embodiment 412, wherein R⁵ is optionally substituted phenyl.

422. The compound of any one of embodiments 1 and 5-357, wherein L² is

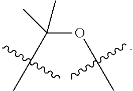 [SMILES: *OC(*)(C)C]

423. The compound of any one of embodiments 1 and 5-357, wherein L² is

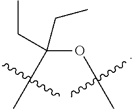 [SMILES: *OC(*)(CC)CC]

424. The compound of any one of embodiments 1 and 5-357, wherein L² is

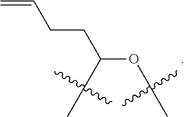 [SMILES: *OC(*)CCC=C]

425. The compound of any one of embodiments 1 and 5-357, wherein L² is

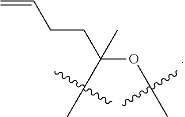 [SMILES: *OC(*)(C)CCC=C]

426. The compound of any one of embodiments 1 and 5-357, wherein L² is

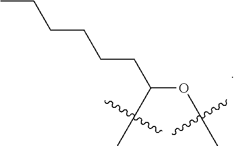 [SMILES: *OC(*)CCCCCC]

427. The compound of any one of embodiments 1 and 5-357, wherein L² is

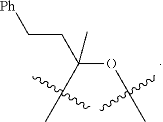 [SMILES: *OC(*)(C)CCc1ccccc1]

428. The compound of any one of embodiments 1 and 5-357, wherein L² is

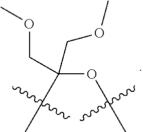 [SMILES: *OC(*)(COC)COC]

429. The compound of any one of embodiments 1 and 5-357, wherein L² is

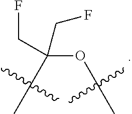 [SMILES: *OC(*)(CF)CF]

430. The compound of any one of embodiments 1 and 5-357, wherein L² is

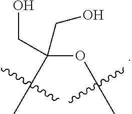 [SMILES: *OC(*)(CO)CO]

431. The compound of any one of embodiments 1 and 5-357, wherein L² is

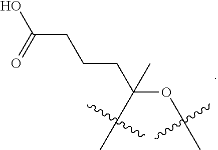 [SMILES: *OC(*)(C)CCCC(=O)O]

432. The compound of any one of embodiments 1 and 5-357, wherein L² is

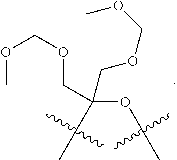 [SMILES: *OC(*)(COCOC)COCOC]

433. The compound of any one of embodiments 1 and 5-357, wherein L² is

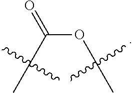 [SMILES: *OC(*)=O]

434. The compound of any one of embodiments 1 and 5-357, wherein L² is optionally substituted C₁₋₃ alkylene, wherein one or more methylene units of the alkylene are independently replaced with —S—.

435. The compound of any one of embodiments 1 and 5-357, wherein L² is optionally substituted C₁₋₃ alkylene, wherein one methylene unit of the alkylene is replaced with —S—.

436. The compound of any one of embodiments 1 and 5-357, wherein L² is optionally substituted —CH₂S—.

437. The compound of any one of embodiments 1 and 5-357, wherein L² is —CH₂S—.

438. The compound of any one of embodiments 1 and 5-357, wherein L² is substituted —CH₂S—.

439. The compound of any one of embodiments 1 and 5-357, wherein L² is —CH₂—NH—.

440. The compound of any one of embodiments 1 and 5-357, wherein L² is

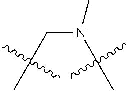 [SMILES: *CN(*)C]

441. The compound of any one of embodiments 1 and 5-357, wherein L² is

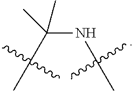 [SMILES: *NC(*)(C)C]

442. The compound of any one of embodiments 1 and 5-357, wherein L² is

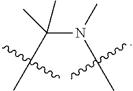 [SMILES: *N(C)C(*)(C)C]

443. The compound of any one of embodiments 1 and 5-357, wherein L² is

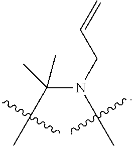 [SMILES: *N(CC=C)C(*)(C)C]

444. The compound of any one of embodiments 1 and 5-357, wherein L² is optionally substituted C₁₋₃ alkylene, wherein one or more methylene units of the alkylene are independently replaced with optionally substituted —CH═N—.

445. The compound of any one of embodiments 1 and 5-357, wherein L² is optionally substituted C₁₋₃ alkylene, wherein one methylene unit of the alkylene is replaced with optionally substituted —CH═N—.

446. The compound of any one of embodiments 1 and 5-357, wherein L² is optionally substituted C₁₋₃ alkylene, wherein one methylene unit of the alkylene is replaced with —CH═N—.

447. The compound of any one of embodiments 1 and 5-357, wherein L² is optionally substituted —CH═N—.

448. The compound of any one of embodiments 1 and 5-357, wherein L² is —CH═N—.

449. The compound of any one of embodiments 1 and 5-357, wherein L² is optionally substituted C₁₋₃ alkylene, wherein one or more methylene units of the alkylene are independently replaced with optionally substituted —CH═CH—.

450. The compound of any one of embodiments 1 and 5-357, wherein L² is optionally substituted C₁₋₃ alkylene, wherein one methylene unit of the alkylene is replaced with optionally substituted —CH═CH—.

451. The compound of any one of embodiments 1 and 5-357, wherein L² is optionally substituted C₁₋₃ alkylene, wherein one methylene unit of the alkylene is replaced with —CH═CH—.

452. The compound of any one of embodiments 1 and 5-357, wherein L² is optionally substituted —CH═CH—.

453. The compound of any one of embodiments 1 and 5-357, wherein L² is —CH═CH—.

454. The compound of any one of embodiments 1 and 5-357, wherein L² is optionally substituted C₁₋₃ alkylene, wherein one or more methylene units of the alkylene are independently replaced with optionally substituted -Cy-.

455. The compound of any one of embodiments 1 and 5-357, wherein L² is optionally substituted C₁₋₃ alkylene, wherein one methylene unit of the alkylene is replaced with -Cy-.

456. The compound of any one of embodiments 1 and 5-357, wherein L² is optionally substituted —CH₂—CH₂—, wherein one methylene unit of —CH₂—CH₂— is replaced with -Cy-.

457. The compound of any one of embodiments 1 and 5-357, wherein L² is -Cy-O—.

458. The compound of embodiment 1, wherein the compound have the structure of

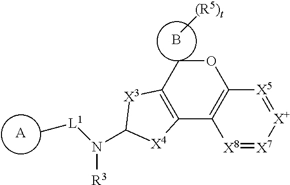

or a salt thereof.

459. The compound of embodiment 1, wherein the compound has the structure of

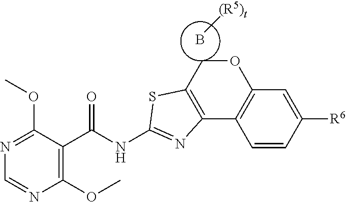

or a salt thereof.

460. The compound of embodiment 1, wherein the compound has the structure of

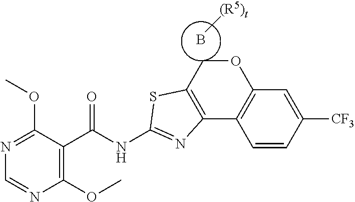

or a salt thereof.

461. The compound of any one of embodiments 454-460, wherein Ring B in the -Cy- is an optionally substituted saturated 3-10 membered ring having 0-4 heteroatoms.

462. The compound of any one of embodiments 454-460, wherein Ring B in the -Cy- is an optionally substituted partially unsaturated 3-10 membered ring having 0-4 heteroatoms.

463. The compound of any one of embodiments 461-462, wherein Ring B is 3-7 membered.

464. The compound of any one of embodiments 461-462, wherein Ring B is 3-membered.

465. The compound of any one of embodiments 461-462, wherein Ring B is 4-membered.

466. The compound of any one of embodiments 461-462, wherein Ring B is 5-membered.

467. The compound of any one of embodiments 461-462, wherein Ring B is 6-membered.

468. The compound of any one of embodiments 461-467, wherein there is no heteroatom in Ring B.

469. The compound of any one of embodiments 461-467, wherein there is one heteroatom in Ring B.

470. The compound of any one of embodiments 461-467, wherein there are two heteroatoms in Ring B.

471. The compound of any one of embodiments 461-462, wherein Ring B is optionally substituted

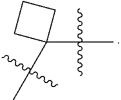 [SMILES: *C1(*)CCC1]

472. The compound of any one of embodiments 461-462, wherein Ring B is optionally substituted

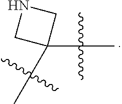 [SMILES: *C1(*)CNC1]

473. The compound of any one of embodiments 461-462, wherein Ring B is optionally substituted

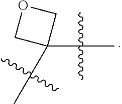 [SMILES: *C1(*)COC1]

474. The compound of any one of embodiments 461-462, wherein Ring B is optionally substituted

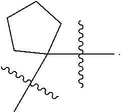 [SMILES: *C1(*)CCCC1]

475. The compound of any one of embodiments 461-462, wherein Ring B is optionally substituted

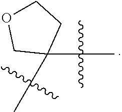 [SMILES: *C1(*)CCOC1]

476. The compound of any one of embodiments 461-462, wherein Ring B is optionally substituted

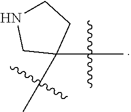 [SMILES: *C1(*)CCNC1]

477. The compound of any one of embodiments 461-462, wherein Ring B is optionally substituted

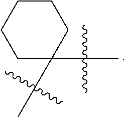 [SMILES: *C1(*)CCCCC1]

478. The compound of any one of embodiments 461-462, wherein Ring B is optionally substituted

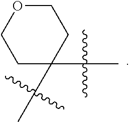 [SMILES: *C1(*)CCOCC1]

479. The compound of any one of embodiments 461-462, wherein Ring B is optionally substituted

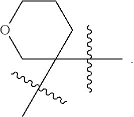 [SMILES: *C1(*)CCCOC1]

480. The compound of any one of embodiments 461-462, wherein Ring B is optionally substituted

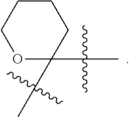 [SMILES: *C1(*)CCCCO1]

481. The compound of any one of embodiments 461-462, wherein Ring B is optionally substituted

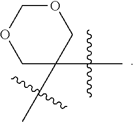 [SMILES: *C1(*)COCOC1]

482. The compound of any one of embodiments 461-462, wherein Ring B is optionally substituted

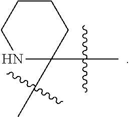 [SMILES: *C1(*)CCCCN1]

483. The compound of any one of embodiments 461-462, wherein Ring B is optionally substituted

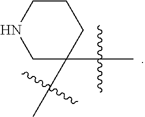 [SMILES: *C1(*)CCCNC1]

484. The compound of any one of embodiments 461-462, wherein Ring B is optionally substituted

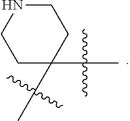 [SMILES: *C1(*)CCNCC1]

485. The compound of any one of embodiments 461-462, wherein Ring B is optionally substituted

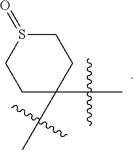 [SMILES: *C1(*)CCS(=O)CC1]

486. The compound of any one of embodiments 461-462, wherein Ring B is optionally substituted

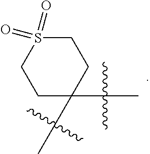 [SMILES: *C1(*)CCS(=O)(=O)CC1]

487. The compound of any one of embodiments 461-462, wherein Ring B is optionally substituted

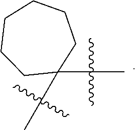 [SMILES: *C1(*)CCCCCC1]

488. The compound of any one of embodiments 461-462, wherein Ring B is optionally substituted

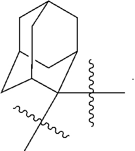 [SMILES: *C1(*)C2CC3CC(C2)CC1C3]

489. The compound of any one of embodiments 454-488, wherein -Cy- is a spirocyclic ring.

490. The compound of any one of embodiments 1 and 5-357, wherein L² is

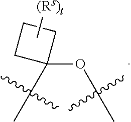

491. The compound of any one of embodiments 1 and 5-357, wherein L² is

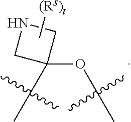

492. The compound of any one of embodiments 1 and 5-357, wherein L² is

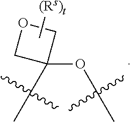

493. The compound of any one of embodiments 1 and 5-357, wherein L² is

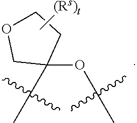

494. The compound of any one of embodiments 1 and 5-357, wherein L² is

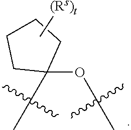

495. The compound of any one of embodiments 1 and 5-357, wherein L² is

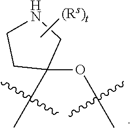

496. The compound of any one of embodiments 1 and 5-357, wherein L² is

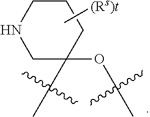

497. The compound of any one of embodiments 1 and 5-357, wherein L² is

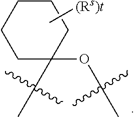

498. The compound of any one of embodiments 1 and 5-357, wherein L² is

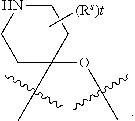

499. The compound of any one of embodiments 1 and 5-357, wherein L² is

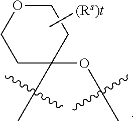

500. The compound of any one of embodiments 1 and 5-357, wherein L² is

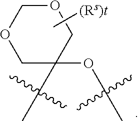

501. The compound of any one of embodiments 1 and 5-357, wherein L² is

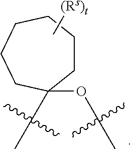

502. The compound of any one of embodiments 1 and 5-357, wherein L² is

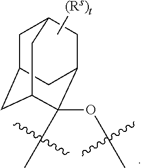

503. The compound of embodiment 1, wherein the compound has the structure of

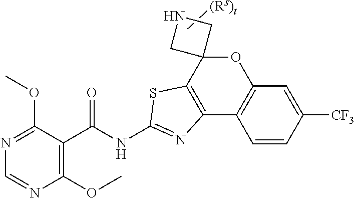

or a salt thereof.

504. The compound of embodiment 1, wherein the compound has a structure of

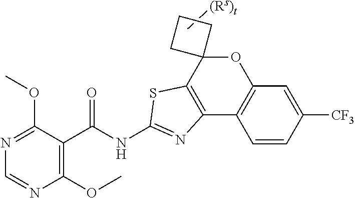

or a salt thereof.

505. The compound of embodiment 1, wherein the compound has a structure of

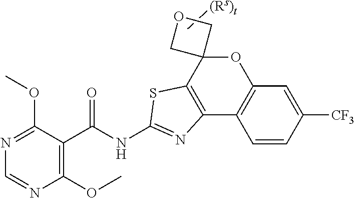

or a salt thereof.

506. The compound of embodiment 1, wherein the compound has a structure of

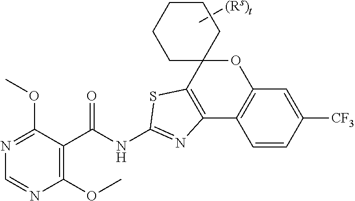

or a salt thereof.

507. The compound of embodiment 1, wherein the compound has a structure of

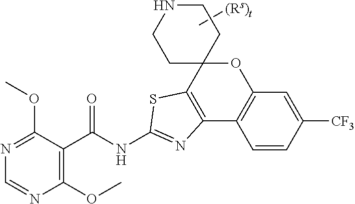

or a salt thereof.

508. The compound of any one of embodiments 1 and 5-507, wherein t is 0.

509. The compound of any one of embodiments 1 and 5-507, wherein t is 1.

510. The compound of any one of embodiments 1 and 5-507, wherein t is 2.

511. The compound of any one of embodiments 1 and 5-507, wherein t is 3.

512 The compound of any one of embodiments 1 and 5-507, wherein t is 4.

513. The compound of any one of embodiments 1 and 5-512, wherein an occurrence of R^{s} is -L-R′.

514. The compound of any one of embodiments 1 and 5-512, wherein an occurrence of R^{s} is -L-R′, wherein L is optionally substituted —CH₂—.

515. The compound of any one of embodiments 1 and 5-512, wherein an occurrence of R^{s} is -L-R′, wherein L is optionally substituted —CH₂—, and R′ is —C(O)OR.

516. The compound of any one of embodiments 1 and 5-512, wherein an occurrence of R^{s} is —CH₂—R′.

517. The compound of any one of embodiments 1 and 5-512, wherein an occurrence of R^{s} is —R′.

518. The compound of any one of embodiments 1 and 5-512, wherein an occurrence of R^{s} is —H.

519. The compound of any one of embodiments 1 and 5-512, wherein an occurrence of R^{s} is optionally substituted C₁₋₆ aliphatic.

520. The compound of any one of embodiments 1 and 5-512, wherein an occurrence of R^{s} is optionally substituted C₁₋₆ alkyl.

521. The compound of any one of embodiments 1 and 5-512, wherein an occurrence of R^{s} is optionally substituted C₃₋₆ cycloalkyl.

522. The compound of any one of embodiments 1 and 5-512, wherein an occurrence of R^{s} is —C(O)OR′.

523. The compound of any one of embodiments 1 and 5-512, wherein an occurrence of R^{s} is —C(O)OR′, wherein R′ is optionally substituted C₁₋₆ aliphatic.

524. The compound of any one of embodiments 1 and 5-512, wherein an occurrence of R^{s} is —C(O)OR′, wherein R′ is optionally substituted C₁₋₆ alkyl.

525. The compound of any one of embodiments 1 and 5-357, wherein L² is

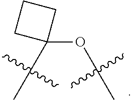 [SMILES: *OC1(*)CCC1]

526. The compound of any one of embodiments 1 and 5-357, wherein L² is

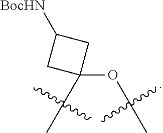 [SMILES: *OC1(*)CC(NC(=O)OC(C)(C)C)C1]

527. The compound of any one of embodiments 1 and 5-357, wherein L² is

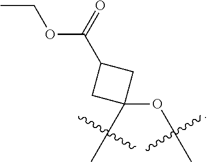 [SMILES: *OC1(*)CC(C(=O)OCC)C1]

528. The compound of any one of embodiments 1 and 5-357, wherein L² is

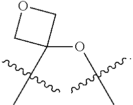 [SMILES: *OC1(*)COC1]

529. The compound of any one of embodiments 1 and 5-357, wherein L² is

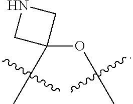 [SMILES: *OC1(*)CNC1]

530. The compound of any one of embodiments 1 and 5-357, wherein L² is

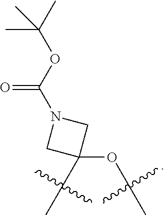 [SMILES: *OC1(*)CN(C(=O)OC(C)(C)C)C1]

531. The compound of any one of embodiments 1 and 5-357, wherein L² is

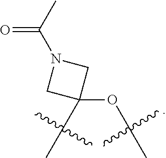 [SMILES: *OC1(*)CN(C(C)=O)C1]

532. The compound of any one of embodiments 1 and 5-357, wherein L² is

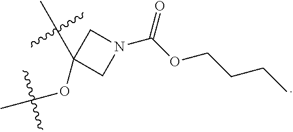 [SMILES: *OC1(*)CN(C(=O)OCCCC)C1]

533. The compound of any one of embodiments 1 and 5-357, wherein L² is

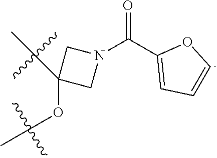 [SMILES: *OC1(*)CN(C(=O)c2ccco2)C1]

534. The compound of any one of embodiments 1 and 5-357, wherein L² is

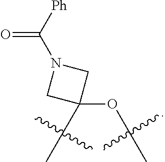 [SMILES: *OC1(*)CN(C(=O)c2ccccc2)C1]

535. The compound of any one of embodiments 1 and 5-357, wherein L² is

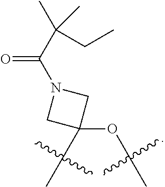 [SMILES: *OC1(*)CN(C(=O)C(C)(C)CC)C1]

536. The compound of any one of embodiments 1 and 5-357, wherein L² is

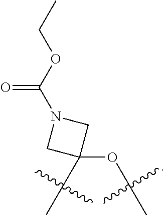 [SMILES: *OC1(*)CN(C(=O)OCC)C1]

537. The compound of any one of embodiments 1 and 5-357, wherein L² is

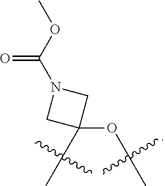 [SMILES: *OC1(*)CN(C(=O)OC)C1]

538. The compound of any one of embodiments 1 and 5-357, wherein L² is

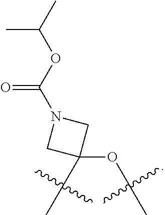 [SMILES: *OC1(*)CN(C(=O)OC(C)C)C1]

539. The compound of any one of embodiments 1 and 5-357, wherein L² is

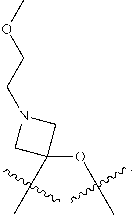 [SMILES: *OC1(*)CN(CCOC)C1]

540. The compound of any one of embodiments 1 and 5-357, wherein L² is

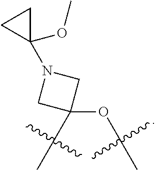 [SMILES: *OC1(*)CN(C2(OC)CC2)C1]

541. The compound of any one of embodiments 1 and 5-357, wherein L² is

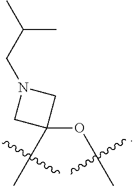 [SMILES: *OC1(*)CN(CC(C)C)C1]

542. The compound of any one of embodiments 1 and 5-357, wherein L² is

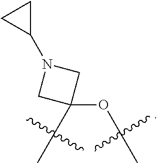 [SMILES: *OC1(*)CN(C2CC2)C1]

543. The compound of any one of embodiments 1 and 5-357, wherein L² is

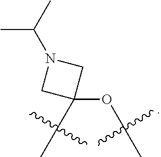 [SMILES: *OC1(*)CN(C(C)C)C1]

544. The compound of any one of embodiments 1 and 5-357, wherein L² is

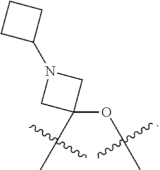 [SMILES: *OC1(*)CN(C2CCC2)C1]

545. The compound of any one of embodiments 1 and 5-357, wherein L² is

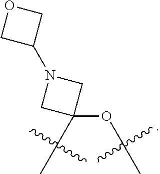 [SMILES: *OC1(*)CN(C2COC2)C1]

546. The compound of any one of embodiments 1 and 5-357, wherein L² is

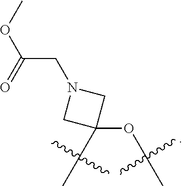 [SMILES: *OC1(*)CN(CC(=O)OC)C1]

547. The compound of any one of embodiments 1 and 5-357, wherein L² is

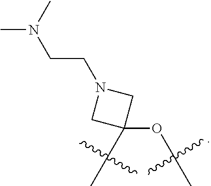 [SMILES: *OC1(*)CN(CCN(C)C)C1]

548. The compound of any one of embodiments 1 and 5-357, wherein L² is

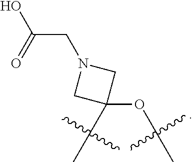 [SMILES: *OC1(*)CN(CC(=O)O)C1]

549. The compound of any one of embodiments 1 and 5-357, wherein L² is

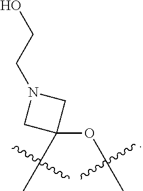 [SMILES: *OC1(*)CN(CCO)C1]

550. The compound of any one of embodiments 1 and 5-357, wherein L² is

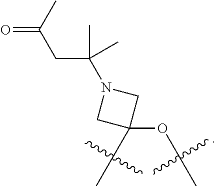 [SMILES: *OC1(*)CN(C(C)(C)CC(C)=O)C1]

551. The compound of any one of embodiments 1 and 5-357, wherein L² is

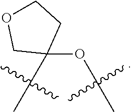 [SMILES: *OC1(*)CCOC1]

552. The compound of any one of embodiments 1 and 5-357, wherein L² is

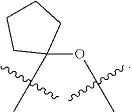 [SMILES: *OC1(*)CCCC1]

553. The compound of any one of embodiments 1 and 5-357, wherein L² is

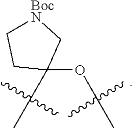 [SMILES: *OC1(*)CCN(C(=O)OC(C)(C)C)C1]

554. The compound of any one of embodiments 1 and 5-357, wherein L² is

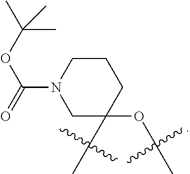 [SMILES: *OC1(*)CCCN(C(=O)OC(C)(C)C)C1]

555. The compound of any one of embodiments 1 and 5-357, wherein L² is

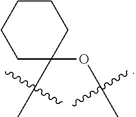 [SMILES: *OC1(*)CCCCC1]

556. The compound of any one of embodiments 1 and 5-357, wherein L² is

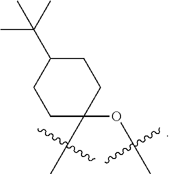 [SMILES: *OC1(*)CCC(C(C)(C)C)CC1]

557. The compound of any one of embodiments 1 and 5-357, wherein L² is

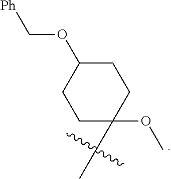 [SMILES: *C1(OC)CCC(OCc2ccccc2)CC1]

558. The compound of any one of embodiments 1 and 5-357, wherein L² is

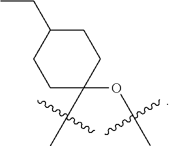 [SMILES: *OC1(*)CCC(CC)CC1]

559. The compound of any one of embodiments 1 and 5-357, wherein L² is

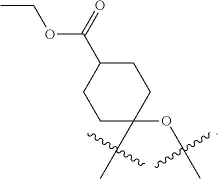 [SMILES: *OC1(*)CCC(C(=O)OCC)CC1]

560. The compound of any one of embodiments 1 and 5-357, wherein L² is

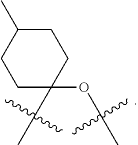 [SMILES: *OC1(*)CCC(C)CC1]

561. The compound of any one of embodiments 1 and 5-357, wherein L² is

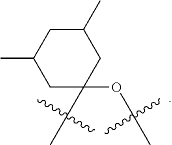 [SMILES: *OC1(*)CC(C)CC(C)C1]

562. The compound of any one of embodiments 1 and 5-357, wherein L² is

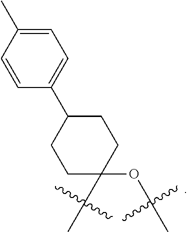 [SMILES: *OC1(*)CCC(c2ccc(C)cc2)CC1]

563. The compound of any one of embodiments 1 and 5-357, wherein L² is

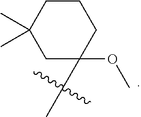

564. The compound of any one of embodiments 1 and 5-357, wherein L² is

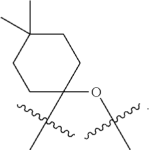 [SMILES: *OC1(*)CCC(C)(C)CC1]

565. The compound of any one of embodiments 1 and 5-357, wherein L² is

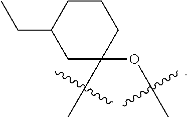 [SMILES: *OC1(*)CCCC(CC)C1]

566. The compound of any one of embodiments 1 and 5-357, wherein L² is

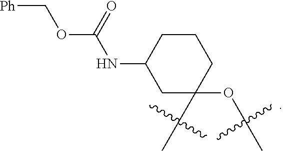 [SMILES: *OC1(*)CCCC(NC(=O)OCc2ccccc2)C1]

567. The compound of any one of embodiments 1 and 5-357, wherein L² is

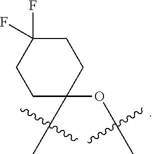 [SMILES: *OC1(*)CCC(F)(F)CC1]

568. The compound of any one of embodiments 1 and 5-357, wherein L² is

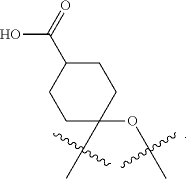 [SMILES: *OC1(*)CCC(C(=O)O)CC1]

569. The compound of any one of embodiments 1 and 5-357, wherein L² is

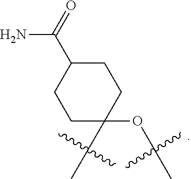 [SMILES: *OC1(*)CCC(C(N)=O)CC1]

570. The compound of any one of embodiments 1 and 5-357, wherein L² is

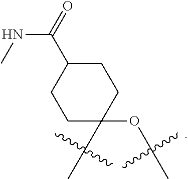 [SMILES: *OC1(*)CCC(C(=O)NC)CC1]

571. The compound of any one of embodiments 1 and 5-357, wherein L² is

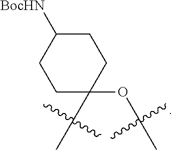 [SMILES: *OC1(*)CCC(NC(=O)OC(C)(C)C)CC1]

572. The compound of any one of embodiments 1 and 5-357, wherein L² is

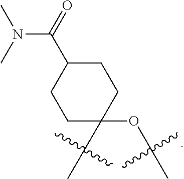 [SMILES: *OC1(*)CCC(C(=O)N(C)C)CC1]

573. The compound of any one of embodiments 1 and 5-357, wherein L² is

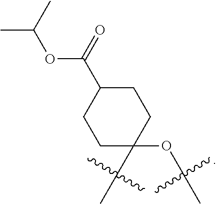 [SMILES: *OC1(*)CCC(C(=O)OC(C)C)CC1]

574. The compound of any one of embodiments 1 and 5-357, wherein L² is

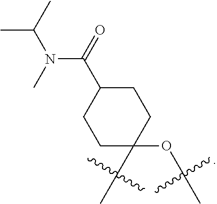 [SMILES: *OC1(*)CCC(C(=O)N(C)C(C)C)CC1]

575. The compound of any one of embodiments 1 and 5-357, wherein L² is

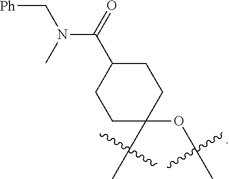 [SMILES: *OC1(*)CCC(C(=O)N(C)Cc2ccccc2)CC1]

576. The compound of any one of embodiments 1 and 5-357, wherein L² is

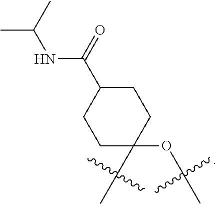 [SMILES: *OC1(*)CCC(C(=O)NC(C)C)CC1]

577. The compound of any one of embodiments 1 and 5-357, wherein L² is

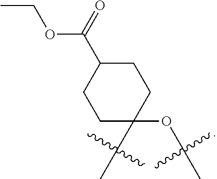 [SMILES: *OC1(*)CCC(C(=O)OCC)CC1]

578. The compound of any one of embodiments 1 and 5-357, wherein L² is

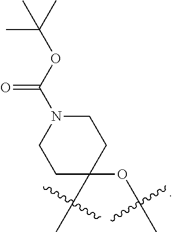 [SMILES: *OC1(*)CCN(C(=O)OC(C)(C)C)CC1]

579. The compound of any one of embodiments 1 and 5-357, wherein L² is

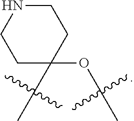 [SMILES: *OC1(*)CCNCC1]

580. The compound of any one of embodiments 1 and 5-357, wherein L² is

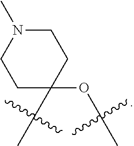 [SMILES: *OC1(*)CCN(C)CC1]

581. The compound of any one of embodiments 1 and 5-357, wherein L² is

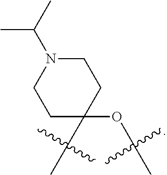 [SMILES: *OC1(*)CCN(C(C)C)CC1]

582. The compound of any one of embodiments 1 and 5-357, wherein L² is

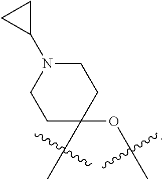 [SMILES: *OC1(*)CCN(C2CC2)CC1]

583. The compound of any one of embodiments 1 and 5-357, wherein L² is

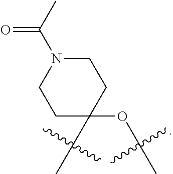 [SMILES: *OC1(*)CCN(C(C)=O)CC1]

584. The compound of any one of embodiments 1 and 5-357, wherein L² is

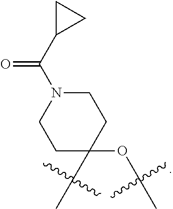 [SMILES: *OC1(*)CCN(C(=O)C2CC2)CC1]

585. The compound of any one of embodiments 1 and 5-357, wherein L² is

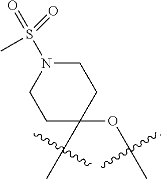 [SMILES: *OC1(*)CCN(S(C)(=O)=O)CC1]

586. The compound of any one of embodiments 1 and 5-357, wherein L² is

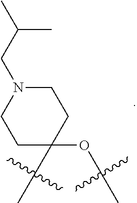 [SMILES: *OC1(*)CCN(CC(C)C)CC1]

587. The compound of any one of embodiments 1 and 5-357, wherein L² is

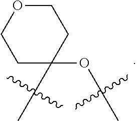 [SMILES: *OC1(*)CCOCC1]

588. The compound of any one of embodiments 1 and 5-357, wherein L² is

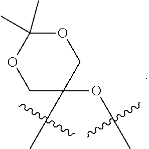 [SMILES: *OC1(*)COC(C)(C)OC1]

589. The compound of any one of embodiments 1 and 5-357, wherein L² is

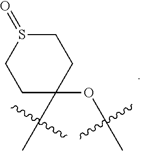 [SMILES: *OC1(*)CCS(=O)CC1]

590. The compound of any one of embodiments 1 and 5-357, wherein L² is

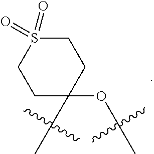 [SMILES: *OC1(*)CCS(=O)(=O)CC1]

591. The compound of any one of embodiments 1 and 5-357, wherein L² is

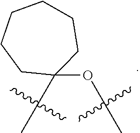 [SMILES: *OC1(*)CCCCCC1]

592. The compound of any one of embodiments 1 and 5-357, wherein L² is

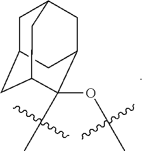 [SMILES: *OC1(*)C2CC3CC(C2)CC1C3]

593. A compound, wherein the compound is selected from:

| Compound | Structure |
|---|---|
| 1 | 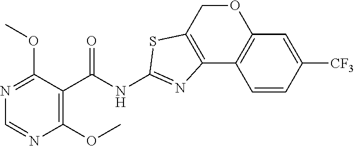 [SMILES: COc1ncnc(OC)c1C(=O)Nc1nc2c(s1)COc1cc(C(F)(F)F)ccc1-2] |
| 2 | 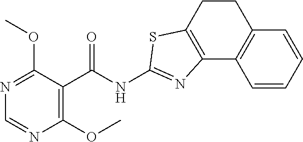 [SMILES: COc1ncnc(OC)c1C(=O)Nc1nc2c(s1)CCc1ccccc1-2] |
| 3 | 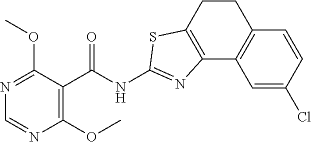 [SMILES: COc1ncnc(OC)c1C(=O)Nc1nc2c(s1)CCc1ccc(Cl)cc1-2] |
| 4 | 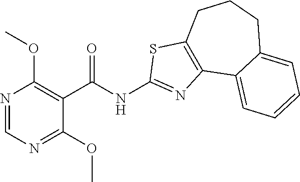 [SMILES: COc1ncnc(OC)c1C(=O)Nc1nc2c(s1)CCCc1ccccc1-2] |
| 5 | 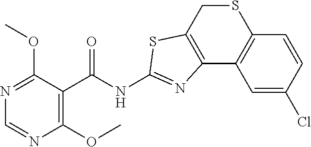 [SMILES: COc1ncnc(OC)c1C(=O)Nc1nc2c(s1)CSc1ccc(Cl)cc1-2] |
| 6 | 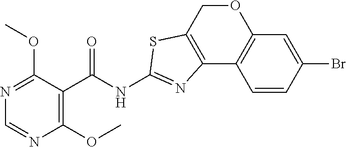 [SMILES: COc1ncnc(OC)c1C(=O)Nc1nc2c(s1)COc1cc(Br)ccc1-2] |
| 7 | 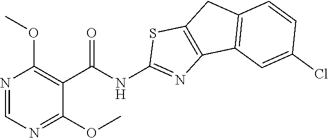 [SMILES: COc1ncnc(OC)c1C(=O)Nc1nc2c(s1)Cc1ccc(Cl)cc1-2] |
| 8 | 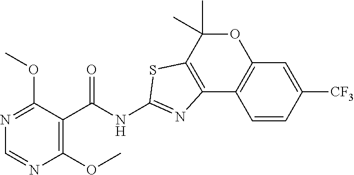 [SMILES: COc1ncnc(OC)c1C(=O)Nc1nc2c(s1)C(C)(C)Oc1cc(C(F)(F)F)ccc1-2] |
| 9 | 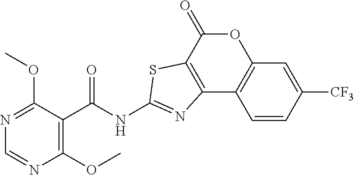 [SMILES: COc1ncnc(OC)c1C(=O)Nc1nc2c(s1)c(=O)oc1cc(C(F)(F)F)ccc12] |
| 10 | 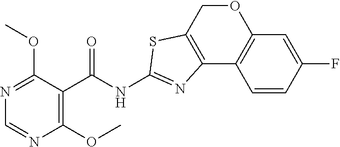 [SMILES: COc1ncnc(OC)c1C(=O)Nc1nc2c(s1)COc1cc(F)ccc1-2] |
| 11 | 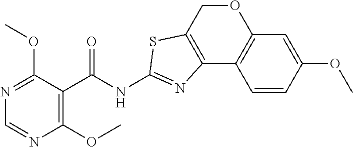 |
| 12 | 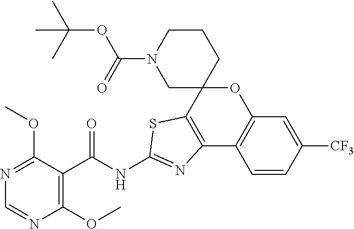 [SMILES: COc1ncnc(OC)c1C(=O)Nc1nc2c(s1)C1(CCCN(C(=O)OC(C)(C)C)C1)Oc1cc(C(F)(F)F)ccc1-2] |
| 13 | 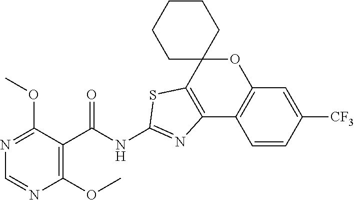 [SMILES: COc1ncnc(OC)c1C(=O)Nc1nc2c(s1)C1(CCCCC1)Oc1cc(C(F)(F)F)ccc1-2] |
| 14 | 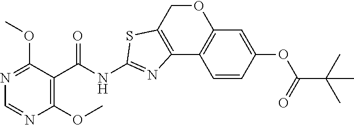 [SMILES: COc1ncnc(OC)c1C(=O)Nc1nc2c(s1)COc1cc(OC(=O)C(C)(C)C)ccc1-2] |
| 15 | 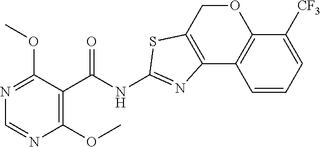 [SMILES: COc1ncnc(OC)c1C(=O)Nc1nc2c(s1)COc1c-2cccc1C(F)(F)F] |
| 16 | 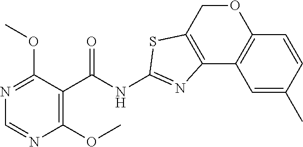 |
| 17 | 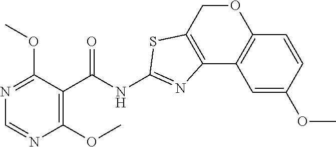 |
| 18 | 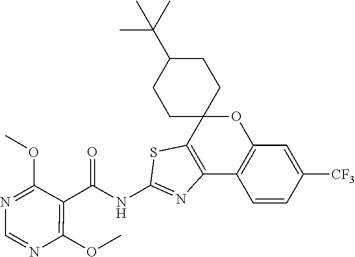 [SMILES: COc1ncnc(OC)c1C(=O)Nc1nc2c(s1)C1(CCC(C(C)(C)C)CC1)Oc1cc(C(F)(F)F)ccc1-2] |
| 19 | 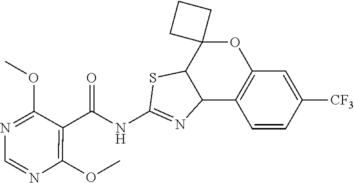 [SMILES: COc1ncnc(OC)c1C(=O)NC1=NC2c3ccc(C(F)(F)F)cc3OC3(CCC3)C2S1] |
| 20 | 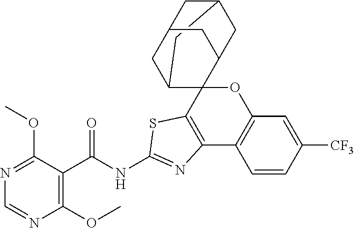 [SMILES: COc1ncnc(OC)c1C(=O)Nc1nc2c(s1)C1(Oc3cc(C(F)(F)F)ccc3-2)C2CC3CC(C2)CC1C3] |
| 21 | 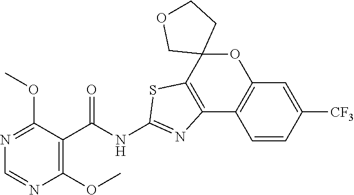 [SMILES: COc1ncnc(OC)c1C(=O)Nc1nc2c(s1)C1(CCOC1)Oc1cc(C(F)(F)F)ccc1-2] |
| 22 | 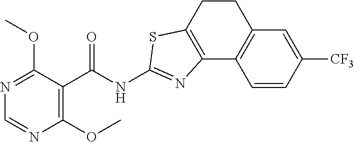 [SMILES: COc1ncnc(OC)c1C(=O)Nc1nc2c(s1)CCc1cc(C(F)(F)F)ccc1-2] |
| 23 | 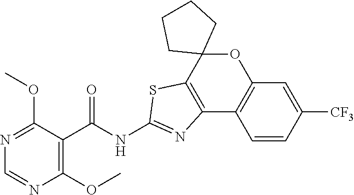 [SMILES: COc1ncnc(OC)c1C(=O)Nc1nc2c(s1)C1(CCCC1)Oc1cc(C(F)(F)F)ccc1-2] |
| 24 | 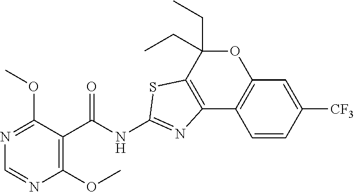 [SMILES: CCC1(CC)Oc2cc(C(F)(F)F)ccc2-c2nc(NC(=O)c3c(OC)ncnc3OC)sc21] |
| 25 | 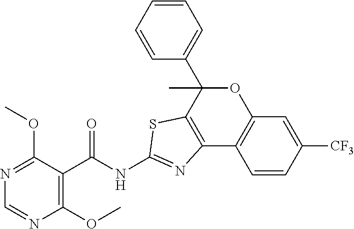 [SMILES: COc1ncnc(OC)c1C(=O)Nc1nc2c(s1)C(C)(c1ccccc1)Oc1cc(C(F)(F)F)ccc1-2] |
| 26 | 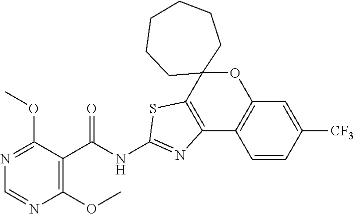 [SMILES: COc1ncnc(OC)c1C(=O)Nc1nc2c(s1)C1(CCCCCC1)Oc1cc(C(F)(F)F)ccc1-2] |
| 27 | 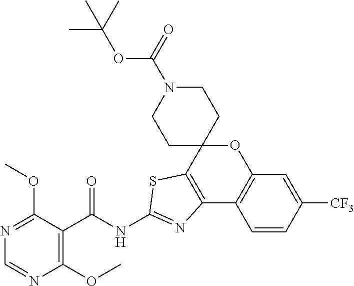 [SMILES: COc1ncnc(OC)c1C(=O)Nc1nc2c(s1)C1(CCN(C(=O)OC(C)(C)C)CC1)Oc1cc(C(F)(F)F)ccc1-2] |
| 28 | 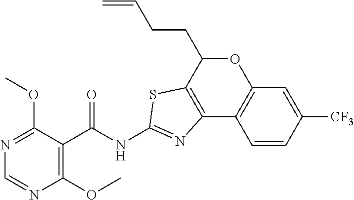 [SMILES: C=CCCC1Oc2cc(C(F)(F)F)ccc2-c2nc(NC(=O)c3c(OC)ncnc3OC)sc21] |
| 29 | 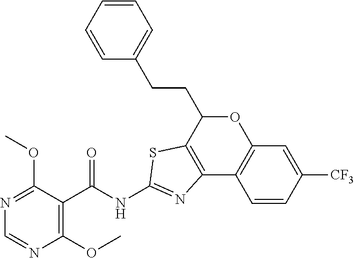 [SMILES: COc1ncnc(OC)c1C(=O)Nc1nc2c(s1)C(CCc1ccccc1)Oc1cc(C(F)(F)F)ccc1-2] |
| 30 | 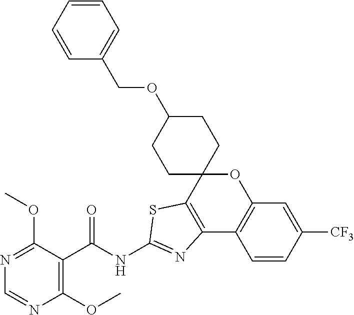 [SMILES: COc1ncnc(OC)c1C(=O)Nc1nc2c(s1)C1(CCC(OCc3ccccc3)CC1)Oc1cc(C(F)(F)F)ccc1-2] |
| 31 | 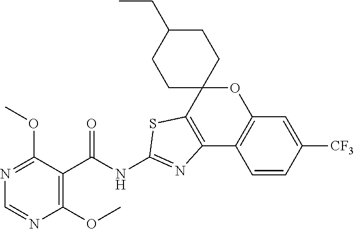 [SMILES: CCC1CCC2(CC1)Oc1cc(C(F)(F)F)ccc1-c1nc(NC(=O)c3c(OC)ncnc3OC)sc12] |
| 32 | 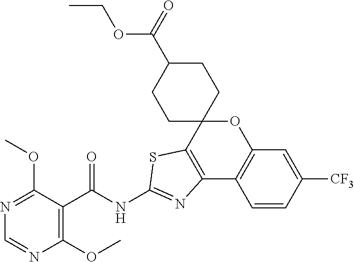 [SMILES: CCOC(=O)C1CCC2(CC1)Oc1cc(C(F)(F)F)ccc1-c1nc(NC(=O)c3c(OC)ncnc3OC)sc12] |
| 33 | 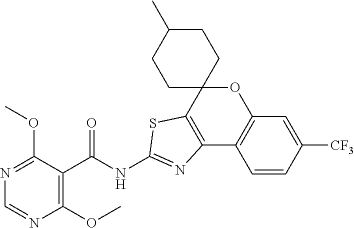 [SMILES: COc1ncnc(OC)c1C(=O)Nc1nc2c(s1)C1(CCC(C)CC1)Oc1cc(C(F)(F)F)ccc1-2] |
| 34 | 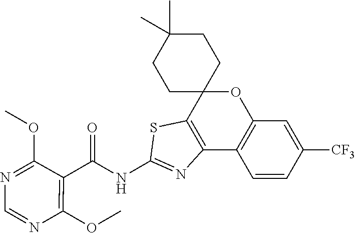 [SMILES: COc1ncnc(OC)c1C(=O)Nc1nc2c(s1)C1(CCC(C)(C)CC1)Oc1cc(C(F)(F)F)ccc1-2] |
| 35 | 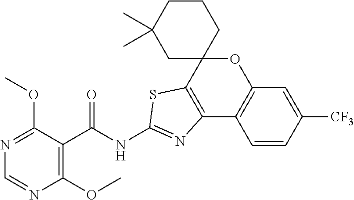 [SMILES: COc1ncnc(OC)c1C(=O)Nc1nc2c(s1)C1(CCCC(C)(C)C1)Oc1cc(C(F)(F)F)ccc1-2] |
| 36 | 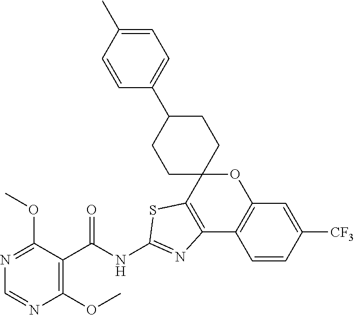 [SMILES: COc1ncnc(OC)c1C(=O)Nc1nc2c(s1)C1(CCC(c3ccc(C)cc3)CC1)Oc1cc(C(F)(F)F)ccc1-2] |
| 37 | 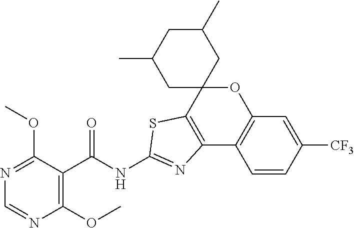 [SMILES: COc1ncnc(OC)c1C(=O)Nc1nc2c(s1)C1(CC(C)CC(C)C1)Oc1cc(C(F)(F)F)ccc1-2] |
| 38 | 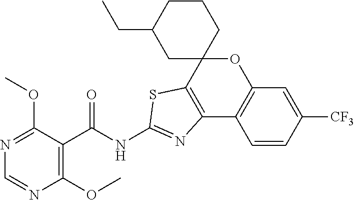 [SMILES: CCC1CCCC2(C1)Oc1cc(C(F)(F)F)ccc1-c1nc(NC(=O)c3c(OC)ncnc3OC)sc12] |
| 39 | 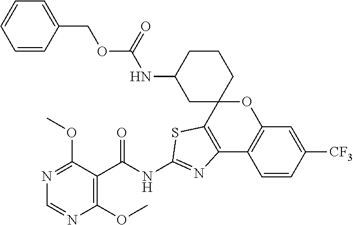 [SMILES: COc1ncnc(OC)c1C(=O)Nc1nc2c(s1)C1(CCCC(NC(=O)OCc3ccccc3)C1)Oc1cc(C(F)(F)F)ccc1-2] |
| 40 | 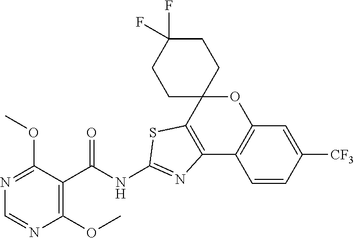 [SMILES: COc1ncnc(OC)c1C(=O)Nc1nc2c(s1)C1(CCC(F)(F)CC1)Oc1cc(C(F)(F)F)ccc1-2] |
| 41 | 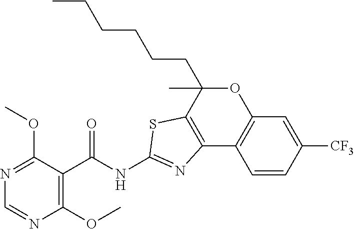 [SMILES: CCCCCCC1(C)Oc2cc(C(F)(F)F)ccc2-c2nc(NC(=O)c3c(OC)ncnc3OC)sc21] |
| 42 | 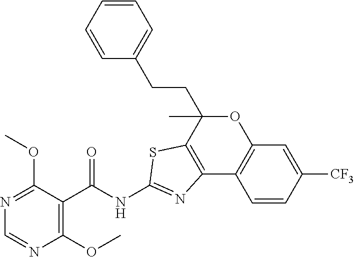 [SMILES: COc1ncnc(OC)c1C(=O)Nc1nc2c(s1)C(C)(CCc1ccccc1)Oc1cc(C(F)(F)F)ccc1-2] |
| 43 | 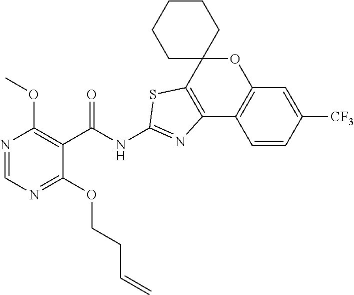 [SMILES: C=CCCOc1ncnc(OC)c1C(=O)Nc1nc2c(s1)C1(CCCCC1)Oc1cc(C(F)(F)F)ccc1-2] |
| 44 | 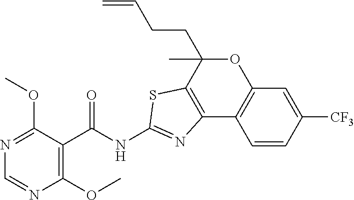 [SMILES: C=CCCC1(C)Oc2cc(C(F)(F)F)ccc2-c2nc(NC(=O)c3c(OC)ncnc3OC)sc21] |
| 45 | 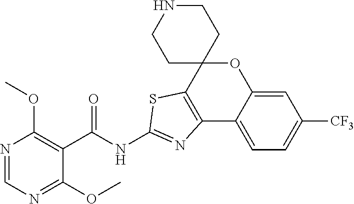 [SMILES: COc1ncnc(OC)c1C(=O)Nc1nc2c(s1)C1(CCNCC1)Oc1cc(C(F)(F)F)ccc1-2] |
| 46 | 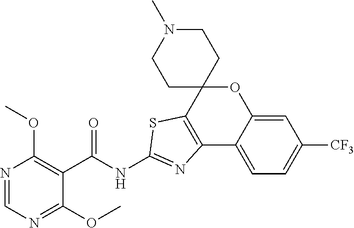 [SMILES: COc1ncnc(OC)c1C(=O)Nc1nc2c(s1)C1(CCN(C)CC1)Oc1cc(C(F)(F)F)ccc1-2] |
| 47 | 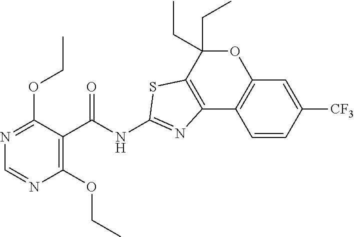 [SMILES: CCOc1ncnc(OCC)c1C(=O)Nc1nc2c(s1)C(CC)(CC)Oc1cc(C(F)(F)F)ccc1-2] |
| 48 | 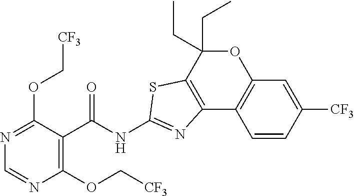 [SMILES: CCC1(CC)Oc2cc(C(F)(F)F)ccc2-c2nc(NC(=O)c3c(OCC(F)(F)F)ncnc3OCC(F)(F)F)sc21] |
| 49 | 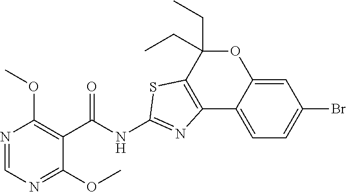 [SMILES: CCC1(CC)Oc2cc(Br)ccc2-c2nc(NC(=O)c3c(OC)ncnc3OC)sc21] |
| 50 | 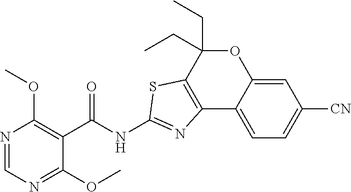 [SMILES: CCC1(CC)Oc2cc(C#N)ccc2-c2nc(NC(=O)c3c(OC)ncnc3OC)sc21] |
| 51 | 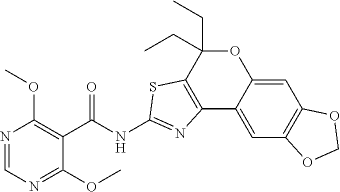 [SMILES: CCC1(CC)Oc2cc3c(cc2-c2nc(NC(=O)c4c(OC)ncnc4OC)sc21)OCO3] |
| 52 | 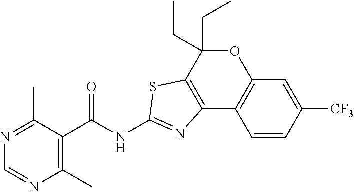 [SMILES: CCC1(CC)Oc2cc(C(F)(F)F)ccc2-c2nc(NC(=O)c3c(C)ncnc3C)sc21] |
| 53 | 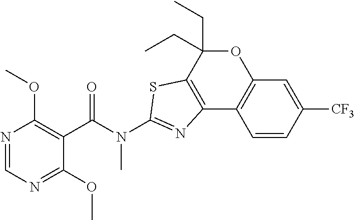 [SMILES: CCC1(CC)Oc2cc(C(F)(F)F)ccc2-c2nc(N(C)C(=O)c3c(OC)ncnc3OC)sc21] |
| 54 | 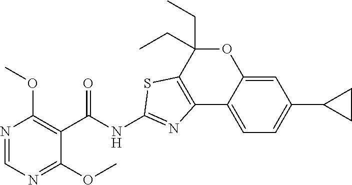 [SMILES: CCC1(CC)Oc2cc(C3CC3)ccc2-c2nc(NC(=O)c3c(OC)ncnc3OC)sc21] |
| 55 | 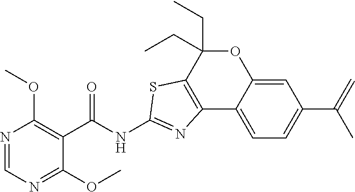 [SMILES: C=C(C)c1ccc2c(c1)OC(CC)(CC)c1sc(NC(=O)c3c(OC)ncnc3OC)nc1-2] |
| 56 | 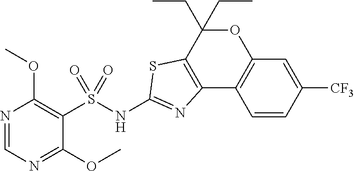 [SMILES: CCC1(CC)Oc2cc(C(F)(F)F)ccc2-c2nc(NS(=O)(=O)c3c(OC)ncnc3OC)sc21] |
| 57 | 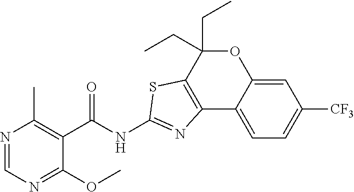 [SMILES: CCC1(CC)Oc2cc(C(F)(F)F)ccc2-c2nc(NC(=O)c3c(C)ncnc3OC)sc21] |
| 58 | 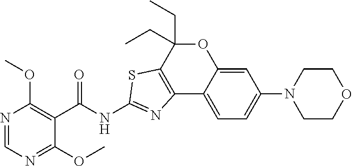 [SMILES: CCC1(CC)Oc2cc(N3CCOCC3)ccc2-c2nc(NC(=O)c3c(OC)ncnc3OC)sc21] |
| 59 | 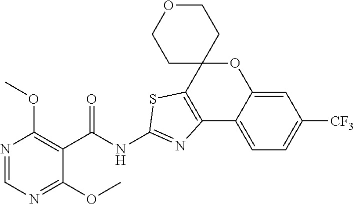 [SMILES: COc1ncnc(OC)c1C(=O)Nc1nc2c(s1)C1(CCOCC1)Oc1cc(C(F)(F)F)ccc1-2] |
| 60 | 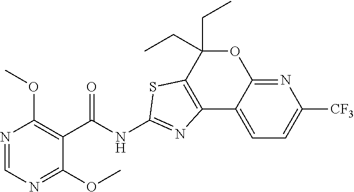 [SMILES: CCC1(CC)Oc2nc(C(F)(F)F)ccc2-c2nc(NC(=O)c3c(OC)ncnc3OC)sc21] |
| 61 | 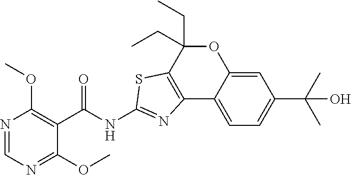 [SMILES: CCC1(CC)Oc2cc(C(C)(C)O)ccc2-c2nc(NC(=O)c3c(OC)ncnc3OC)sc21] |
| 62 | 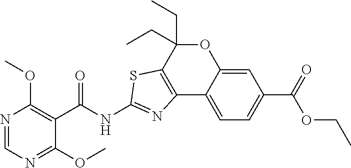 [SMILES: CCOC(=O)c1ccc2c(c1)OC(CC)(CC)c1sc(NC(=O)c3c(OC)ncnc3OC)nc1-2] |
| 63 | 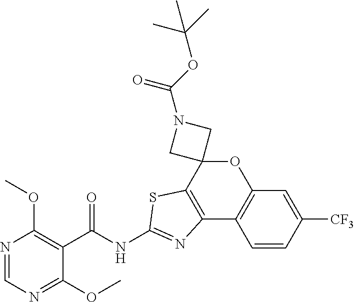 [SMILES: COc1ncnc(OC)c1C(=O)Nc1nc2c(s1)C1(CN(C(=O)OC(C)(C)C)C1)Oc1cc(C(F)(F)F)ccc1-2] |
| 64 | 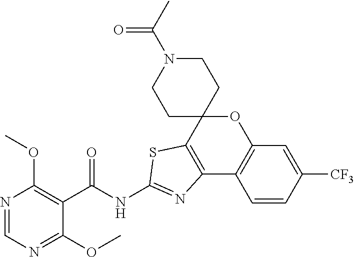 [SMILES: COc1ncnc(OC)c1C(=O)Nc1nc2c(s1)C1(CCN(C(C)=O)CC1)Oc1cc(C(F)(F)F)ccc1-2] |
| 65 | 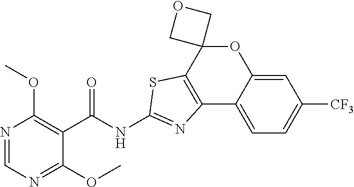 [SMILES: COc1ncnc(OC)c1C(=O)Nc1nc2c(s1)C1(COC1)Oc1cc(C(F)(F)F)ccc1-2] |
| 66 | 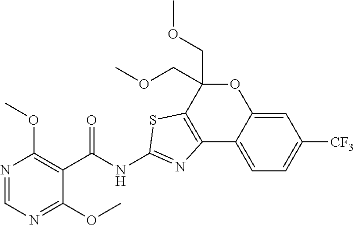 [SMILES: COCC1(COC)Oc2cc(C(F)(F)F)ccc2-c2nc(NC(=O)c3c(OC)ncnc3OC)sc21] |
| 67 | 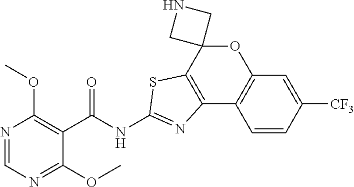 [SMILES: COc1ncnc(OC)c1C(=O)Nc1nc2c(s1)C1(CNC1)Oc1cc(C(F)(F)F)ccc1-2] |
| 68 | 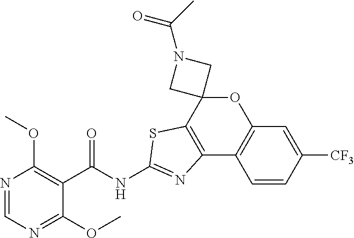 [SMILES: COc1ncnc(OC)c1C(=O)Nc1nc2c(s1)C1(CN(C(C)=O)C1)Oc1cc(C(F)(F)F)ccc1-2] |
| 69 | 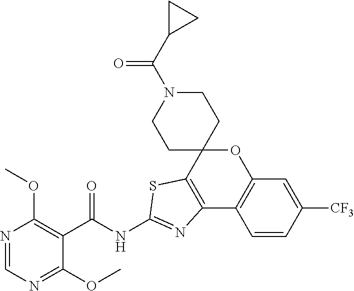 [SMILES: COc1ncnc(OC)c1C(=O)Nc1nc2c(s1)C1(CCN(C(=O)C3CC3)CC1)Oc1cc(C(F)(F)F)ccc1-2] |
| 70 | 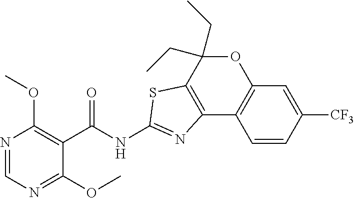 [SMILES: CCC1(CC)Oc2cc(C(F)(F)F)ccc2-c2nc(NC(=O)c3c(OC)ncnc3OC)sc21] |
| 71 | 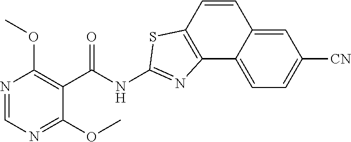 [SMILES: COc1ncnc(OC)c1C(=O)Nc1nc2c(ccc3cc(C#N)ccc32)s1] |
| 72 | 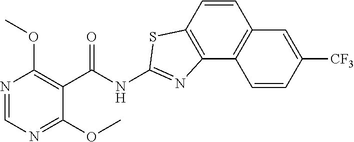 [SMILES: COc1ncnc(OC)c1C(=O)Nc1nc2c(ccc3cc(C(F)(F)F)ccc32)s1] |
| 73 | 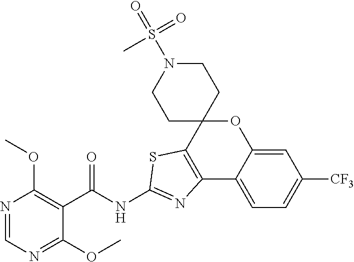 [SMILES: COc1ncnc(OC)c1C(=O)Nc1nc2c(s1)C1(CCN(S(C)(=O)=O)CC1)Oc1cc(C(F)(F)F)ccc1-2] |
| 74 | 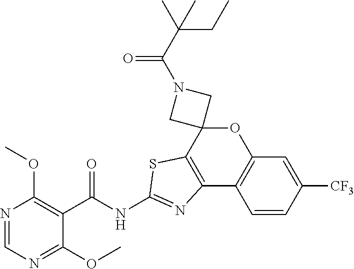 [SMILES: CCC(C)(C)C(=O)N1CC2(C1)Oc1cc(C(F)(F)F)ccc1-c1nc(NC(=O)c3c(OC)ncnc3OC)sc12] |
| 75 | 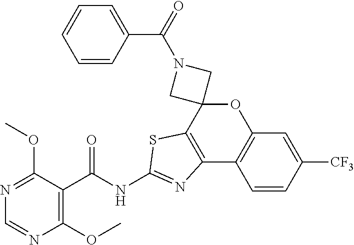 [SMILES: COc1ncnc(OC)c1C(=O)Nc1nc2c(s1)C1(CN(C(=O)c3ccccc3)C1)Oc1cc(C(F)(F)F)ccc1-2] |
| 76 | 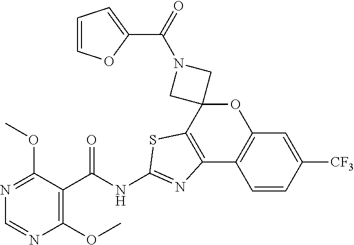 [SMILES: COc1ncnc(OC)c1C(=O)Nc1nc2c(s1)C1(CN(C(=O)c3ccco3)C1)Oc1cc(C(F)(F)F)ccc1-2] |
| 77 | 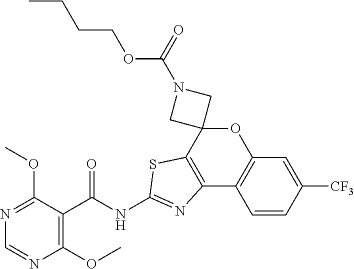 [SMILES: CCCCOC(=O)N1CC2(C1)Oc1cc(C(F)(F)F)ccc1-c1nc(NC(=O)c3c(OC)ncnc3OC)sc12] |
| 78 | 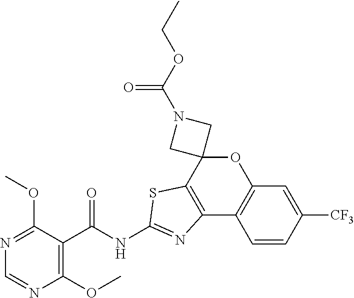 [SMILES: CCOC(=O)N1CC2(C1)Oc1cc(C(F)(F)F)ccc1-c1nc(NC(=O)c3c(OC)ncnc3OC)sc12] |
| 79 | 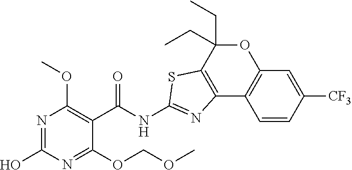 [SMILES: CCC1(CC)Oc2cc(C(F)(F)F)ccc2-c2nc(NC(=O)c3c(OC)nc(O)nc3OCOC)sc21] |
| 80 | 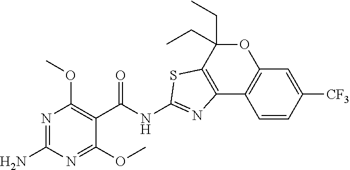 [SMILES: CCC1(CC)Oc2cc(C(F)(F)F)ccc2-c2nc(NC(=O)c3c(OC)nc(N)nc3OC)sc21] |
| 81 | 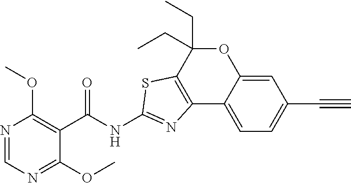 [SMILES: C#Cc1ccc2c(c1)OC(CC)(CC)c1sc(NC(=O)c3c(OC)ncnc3OC)nc1-2] |
| 82 | 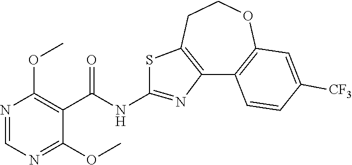 [SMILES: COc1ncnc(OC)c1C(=O)Nc1nc2c(s1)CCOc1cc(C(F)(F)F)ccc1-2] |
| 83 | 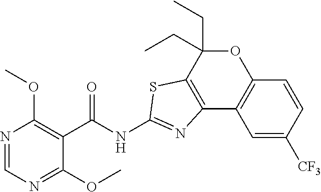 [SMILES: CCC1(CC)Oc2ccc(C(F)(F)F)cc2-c2nc(NC(=O)c3c(OC)ncnc3OC)sc21] |
| 84 | 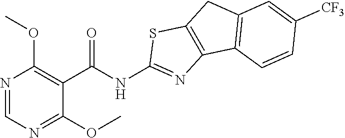 [SMILES: COc1ncnc(OC)c1C(=O)Nc1nc2c(s1)Cc1cc(C(F)(F)F)ccc1-2] |
| 85 | 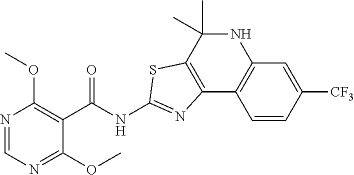 [SMILES: COc1ncnc(OC)c1C(=O)Nc1nc2c(s1)C(C)(C)Nc1cc(C(F)(F)F)ccc1-2] |
| 86 | 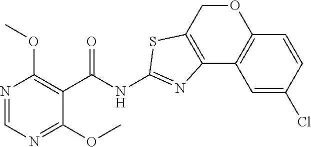 [SMILES: COc1ncnc(OC)c1C(=O)Nc1nc2c(s1)COc1ccc(Cl)cc1-2] |
| 87 | 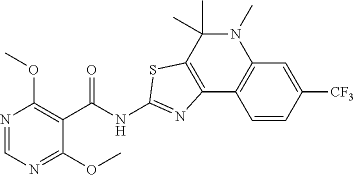 [SMILES: COc1ncnc(OC)c1C(=O)Nc1nc2c(s1)C(C)(C)N(C)c1cc(C(F)(F)F)ccc1-2] |
| 88 | 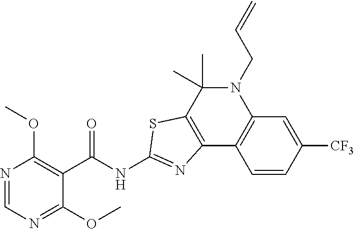 [SMILES: C=CCN1c2cc(C(F)(F)F)ccc2-c2nc(NC(=O)c3c(OC)ncnc3OC)sc2C1(C)C] |
| 89 | 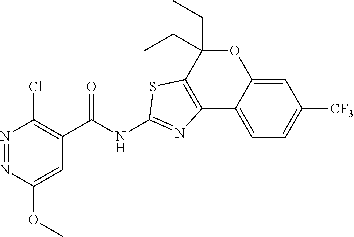 [SMILES: CCC1(CC)Oc2cc(C(F)(F)F)ccc2-c2nc(NC(=O)c3cc(OC)nnc3Cl)sc21] |
| 90 | 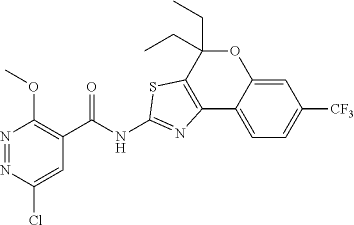 [SMILES: CCC1(CC)Oc2cc(C(F)(F)F)ccc2-c2nc(NC(=O)c3cc(Cl)nnc3OC)sc21] |
| 91 | 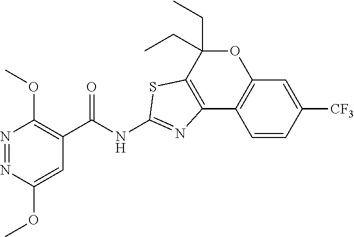 [SMILES: CCC1(CC)Oc2cc(C(F)(F)F)ccc2-c2nc(NC(=O)c3cc(OC)nnc3OC)sc21] |
| 92 | 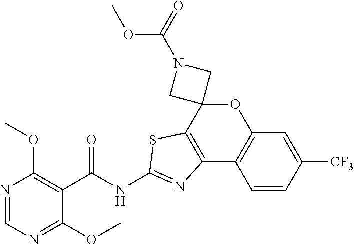 [SMILES: COC(=O)N1CC2(C1)Oc1cc(C(F)(F)F)ccc1-c1nc(NC(=O)c3c(OC)ncnc3OC)sc12] |
| 93 | 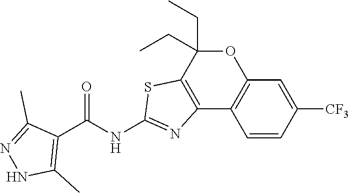 [SMILES: CCC1(CC)Oc2cc(C(F)(F)F)ccc2-c2nc(NC(=O)c3c(C)n[nH]c3C)sc21] |
| 94 | 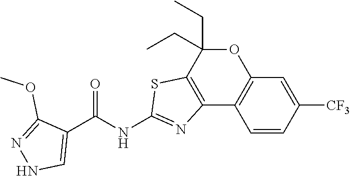 [SMILES: CCC1(CC)Oc2cc(C(F)(F)F)ccc2-c2nc(NC(=O)c3c[nH]nc3OC)sc21] |
| 95 | 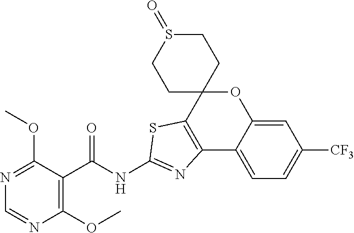 [SMILES: COc1ncnc(OC)c1C(=O)Nc1nc2c(s1)C1(CCS(=O)CC1)Oc1cc(C(F)(F)F)ccc1-2] |
| 96 | 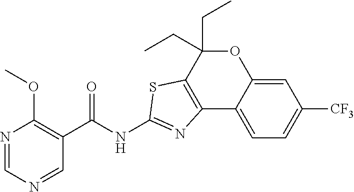 [SMILES: CCC1(CC)Oc2cc(C(F)(F)F)ccc2-c2nc(NC(=O)c3cncnc3OC)sc21] |
| 97 | 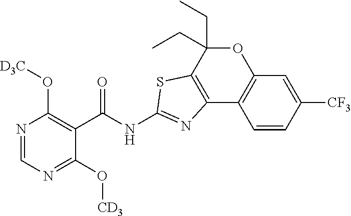 [SMILES: [2H]C([2H])([2H])Oc1ncnc(OC([2H])([2H])[2H])c1C(=O)Nc1nc2c(s1)C(CC)(CC)Oc1cc(C(F)(F)F)ccc1-2] |
| 98 | 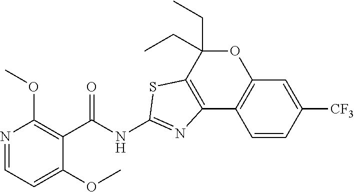 [SMILES: CCC1(CC)Oc2cc(C(F)(F)F)ccc2-c2nc(NC(=O)c3c(OC)ccnc3OC)sc21] |
| 99 | 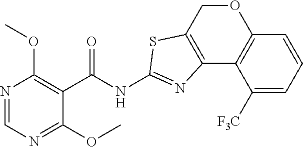 [SMILES: COc1ncnc(OC)c1C(=O)Nc1nc2c(s1)COc1cccc(C(F)(F)F)c1-2] |
| 100 | 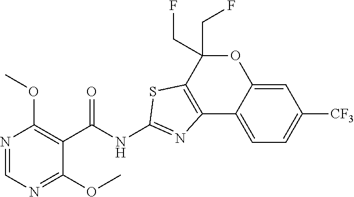 [SMILES: COc1ncnc(OC)c1C(=O)Nc1nc2c(s1)C(CF)(CF)Oc1cc(C(F)(F)F)ccc1-2] |
| 101 | 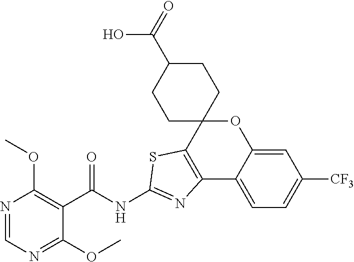 [SMILES: COc1ncnc(OC)c1C(=O)Nc1nc2c(s1)C1(CCC(C(=O)O)CC1)Oc1cc(C(F)(F)F)ccc1-2] |
| 102 | 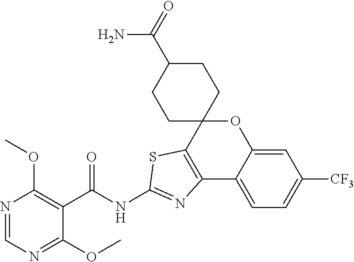 [SMILES: COc1ncnc(OC)c1C(=O)Nc1nc2c(s1)C1(CCC(C(N)=O)CC1)Oc1cc(C(F)(F)F)ccc1-2] |
| 103 | 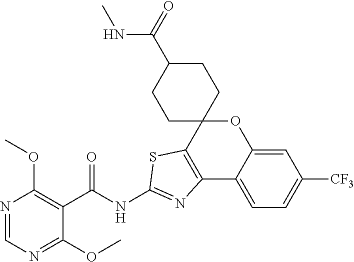 [SMILES: CNC(=O)C1CCC2(CC1)Oc1cc(C(F)(F)F)ccc1-c1nc(NC(=O)c3c(OC)ncnc3OC)sc12] |
| 104 | 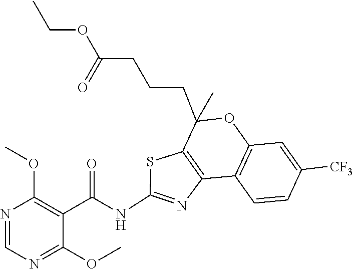 [SMILES: CCOC(=O)CCCC1(C)Oc2cc(C(F)(F)F)ccc2-c2nc(NC(=O)c3c(OC)ncnc3OC)sc21] |
| 105 | 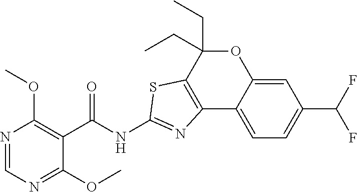 [SMILES: CCC1(CC)Oc2cc(C(F)F)ccc2-c2nc(NC(=O)c3c(OC)ncnc3OC)sc21] |
| 106 | 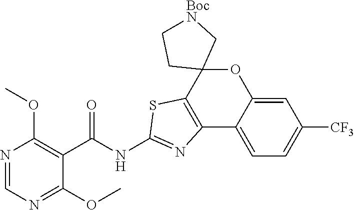 [SMILES: COc1ncnc(OC)c1C(=O)Nc1nc2c(s1)C1(CCN(C(=O)OC(C)(C)C)C1)Oc1cc(C(F)(F)F)ccc1-2] |
| 107 | 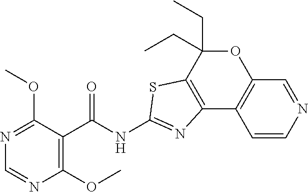 [SMILES: CCC1(CC)Oc2cnccc2-c2nc(NC(=O)c3c(OC)ncnc3OC)sc21] |
| 108 | 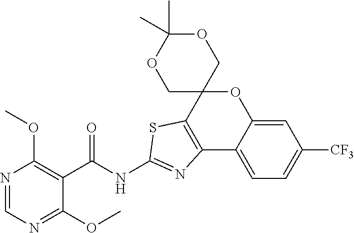 [SMILES: COc1ncnc(OC)c1C(=O)Nc1nc2c(s1)C1(COC(C)(C)OC1)Oc1cc(C(F)(F)F)ccc1-2] |
| 109 | 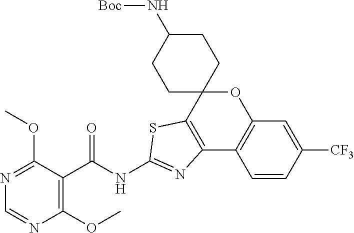 [SMILES: COc1ncnc(OC)c1C(=O)Nc1nc2c(s1)C1(CCC(NC(=O)OC(C)(C)C)CC1)Oc1cc(C(F)(F)F)ccc1-2] |
| 110 | 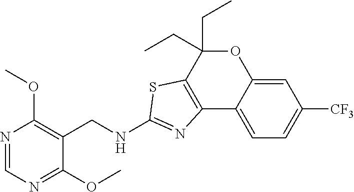 [SMILES: CCC1(CC)Oc2cc(C(F)(F)F)ccc2-c2nc(NCc3c(OC)ncnc3OC)sc21] |
| 111 | 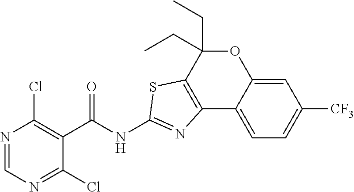 [SMILES: CCC1(CC)Oc2cc(C(F)(F)F)ccc2-c2nc(NC(=O)c3c(Cl)ncnc3Cl)sc21] |
| 112 | 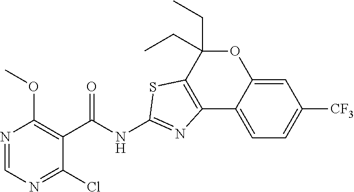 [SMILES: CCC1(CC)Oc2cc(C(F)(F)F)ccc2-c2nc(NC(=O)c3c(Cl)ncnc3OC)sc21] |
| 113 | 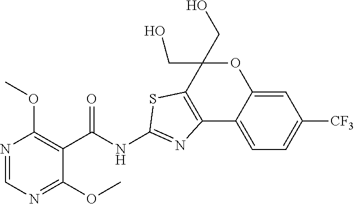 [SMILES: COc1ncnc(OC)c1C(=O)Nc1nc2c(s1)C(CO)(CO)Oc1cc(C(F)(F)F)ccc1-2] |
| 114 | 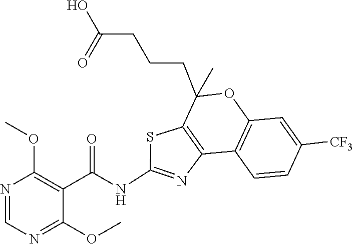 [SMILES: COc1ncnc(OC)c1C(=O)Nc1nc2c(s1)C(C)(CCCC(=O)O)Oc1cc(C(F)(F)F)ccc1-2] |
| 115 | 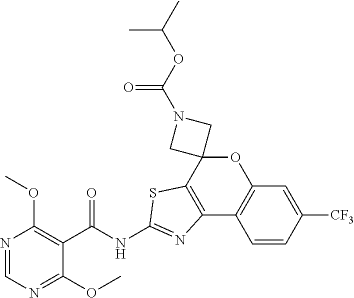 [SMILES: COc1ncnc(OC)c1C(=O)Nc1nc2c(s1)C1(CN(C(=O)OC(C)C)C1)Oc1cc(C(F)(F)F)ccc1-2] |
| 116 | 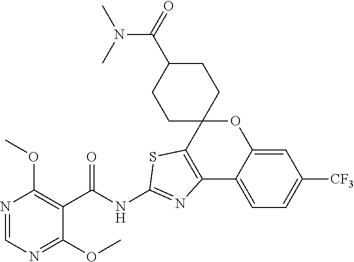 [SMILES: COc1ncnc(OC)c1C(=O)Nc1nc2c(s1)C1(CCC(C(=O)N(C)C)CC1)Oc1cc(C(F)(F)F)ccc1-2] |
| 117 | 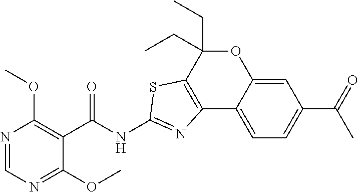 [SMILES: CCC1(CC)Oc2cc(C(C)=O)ccc2-c2nc(NC(=O)c3c(OC)ncnc3OC)sc21] |
| 118 | 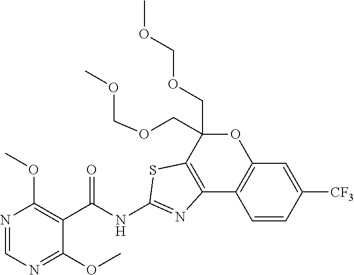 [SMILES: COCOCC1(COCOC)Oc2cc(C(F)(F)F)ccc2-c2nc(NC(=O)c3c(OC)ncnc3OC)sc21] |
| 119 | 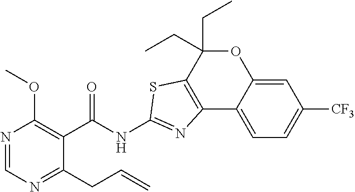 [SMILES: C=CCc1ncnc(OC)c1C(=O)Nc1nc2c(s1)C(CC)(CC)Oc1cc(C(F)(F)F)ccc1-2] |
| 120 | 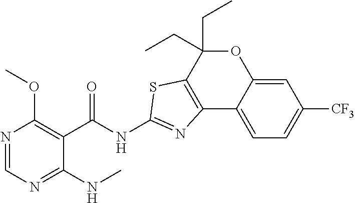 [SMILES: CCC1(CC)Oc2cc(C(F)(F)F)ccc2-c2nc(NC(=O)c3c(NC)ncnc3OC)sc21] |
| 121 | 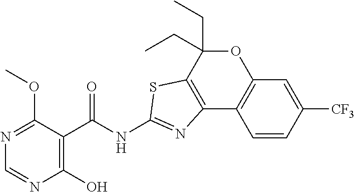 [SMILES: CCC1(CC)Oc2cc(C(F)(F)F)ccc2-c2nc(NC(=O)c3c(O)ncnc3OC)sc21] |
| 122 | 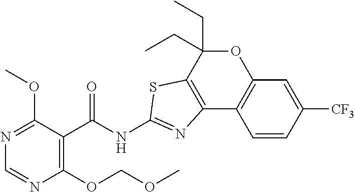 [SMILES: CCC1(CC)Oc2cc(C(F)(F)F)ccc2-c2nc(NC(=O)c3c(OC)ncnc3OCOC)sc21] |
| 123 | 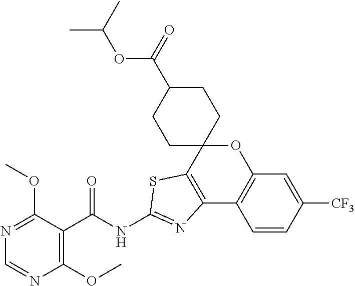 [SMILES: COc1ncnc(OC)c1C(=O)Nc1nc2c(s1)C1(CCC(C(=O)OC(C)C)CC1)Oc1cc(C(F)(F)F)ccc1-2] |
| 124 | 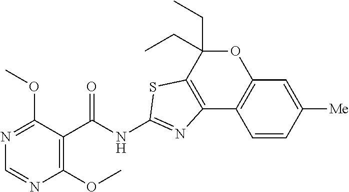 [SMILES: CCC1(CC)Oc2cc(C)ccc2-c2nc(NC(=O)c3c(OC)ncnc3OC)sc21] |
| 125 | 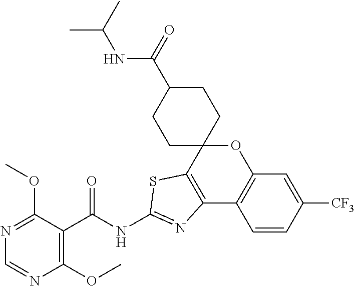 [SMILES: COc1ncnc(OC)c1C(=O)Nc1nc2c(s1)C1(CCC(C(=O)NC(C)C)CC1)Oc1cc(C(F)(F)F)ccc1-2] |
| 126 | 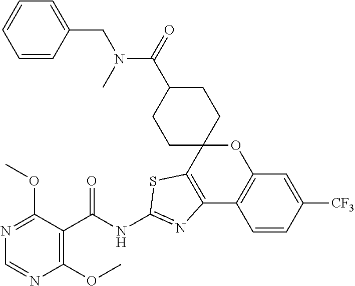 [SMILES: COc1ncnc(OC)c1C(=O)Nc1nc2c(s1)C1(CCC(C(=O)N(C)Cc3ccccc3)CC1)Oc1cc(C(F)(F)F)ccc1-2] |
| 127 | 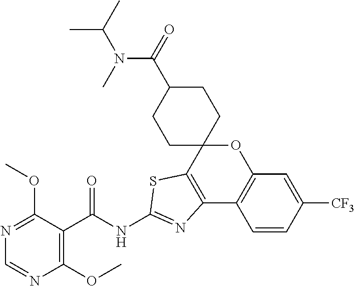 [SMILES: COc1ncnc(OC)c1C(=O)Nc1nc2c(s1)C1(CCC(C(=O)N(C)C(C)C)CC1)Oc1cc(C(F)(F)F)ccc1-2] |
| 128 | 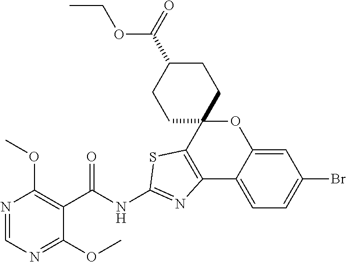 |
| 129 | 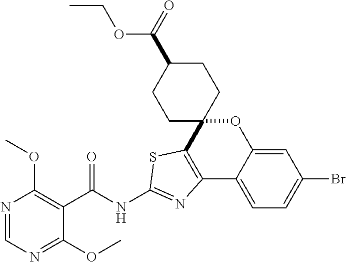 |
| 130 | 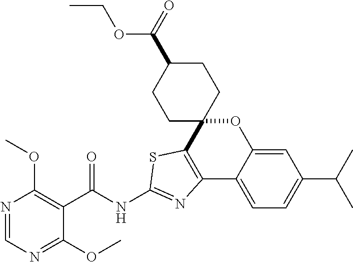 |
| 131 | 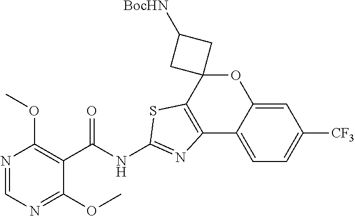 [SMILES: COc1ncnc(OC)c1C(=O)Nc1nc2c(s1)C1(CC(NC(=O)OC(C)(C)C)C1)Oc1cc(C(F)(F)F)ccc1-2] |
| 132 | 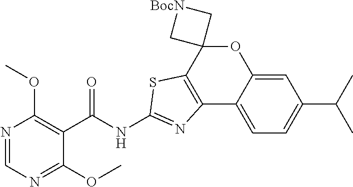 [SMILES: COc1ncnc(OC)c1C(=O)Nc1nc2c(s1)C1(CN(C(=O)OC(C)(C)C)C1)Oc1cc(C(C)C)ccc1-2] |
| 133 | 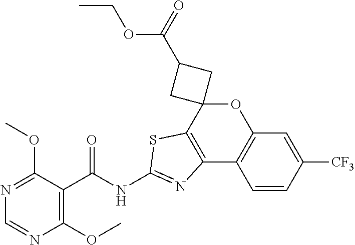 [SMILES: CCOC(=O)C1CC2(C1)Oc1cc(C(F)(F)F)ccc1-c1nc(NC(=O)c3c(OC)ncnc3OC)sc12] |
| 134 | 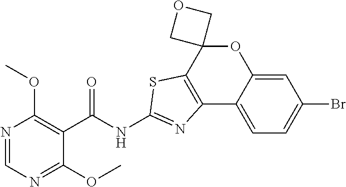 [SMILES: COc1ncnc(OC)c1C(=O)Nc1nc2c(s1)C1(COC1)Oc1cc(Br)ccc1-2] |
| 135 | 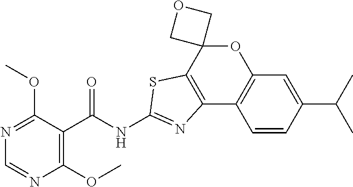 [SMILES: COc1ncnc(OC)c1C(=O)Nc1nc2c(s1)C1(COC1)Oc1cc(C(C)C)ccc1-2] |
| 136 | 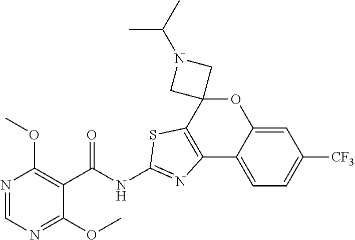 [SMILES: COc1ncnc(OC)c1C(=O)Nc1nc2c(s1)C1(CN(C(C)C)C1)Oc1cc(C(F)(F)F)ccc1-2] |
| 137 | 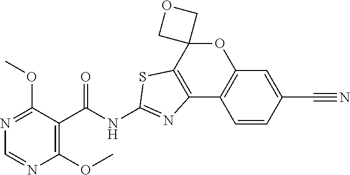 [SMILES: COc1ncnc(OC)c1C(=O)Nc1nc2c(s1)C1(COC1)Oc1cc(C#N)ccc1-2] |
| 138 | 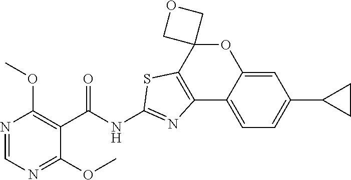 [SMILES: COc1ncnc(OC)c1C(=O)Nc1nc2c(s1)C1(COC1)Oc1cc(C3CC3)ccc1-2] |
| 139 | 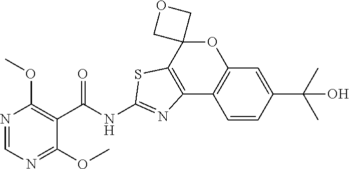 [SMILES: COc1ncnc(OC)c1C(=O)Nc1nc2c(s1)C1(COC1)Oc1cc(C(C)(C)O)ccc1-2] |
| 140 | 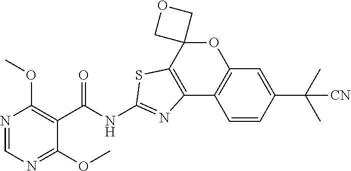 [SMILES: COc1ncnc(OC)c1C(=O)Nc1nc2c(s1)C1(COC1)Oc1cc(C(C)(C)C#N)ccc1-2] |
| 141 | 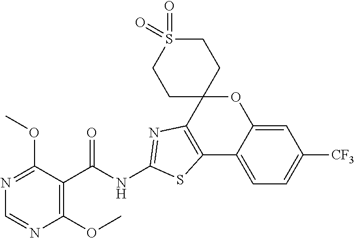 [SMILES: COc1ncnc(OC)c1C(=O)Nc1nc2c(s1)-c1ccc(C(F)(F)F)cc1OC21CCS(=O)(=O)CC1] |
| 142 | 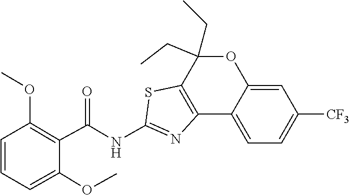 [SMILES: CCC1(CC)Oc2cc(C(F)(F)F)ccc2-c2nc(NC(=O)c3c(OC)cccc3OC)sc21] |
| 143 | 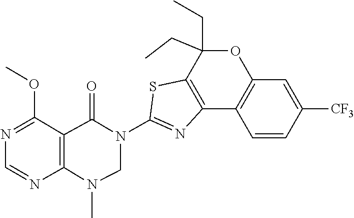 [SMILES: CCC1(CC)Oc2cc(C(F)(F)F)ccc2-c2nc(N3CN(C)c4ncnc(OC)c4C3=O)sc21] |
| 144 | 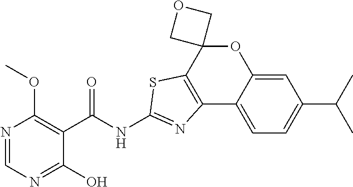 [SMILES: COc1ncnc(O)c1C(=O)Nc1nc2c(s1)C1(COC1)Oc1cc(C(C)C)ccc1-2] |
| 145 | 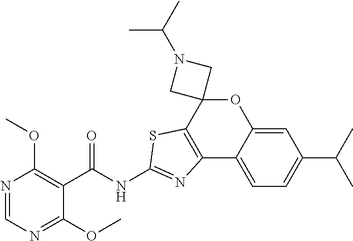 [SMILES: COc1ncnc(OC)c1C(=O)Nc1nc2c(s1)C1(CN(C(C)C)C1)Oc1cc(C(C)C)ccc1-2] |
| 146 | 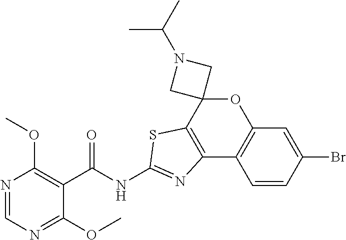 [SMILES: COc1ncnc(OC)c1C(=O)Nc1nc2c(s1)C1(CN(C(C)C)C1)Oc1cc(Br)ccc1-2] |
| 147 | 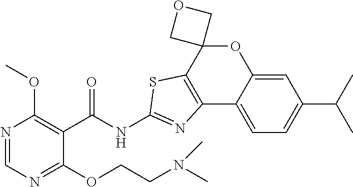 [SMILES: COc1ncnc(OCCN(C)C)c1C(=O)Nc1nc2c(s1)C1(COC1)Oc1cc(C(C)C)ccc1-2] |
| 148 | 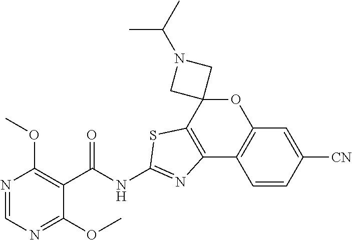 [SMILES: COc1ncnc(OC)c1C(=O)Nc1nc2c(s1)C1(CN(C(C)C)C1)Oc1cc(C#N)ccc1-2] |
| 149 | 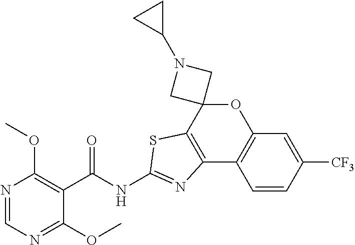 [SMILES: COc1ncnc(OC)c1C(=O)Nc1nc2c(s1)C1(CN(C3CC3)C1)Oc1cc(C(F)(F)F)ccc1-2] |
| 150 | 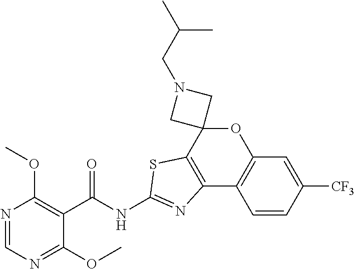 [SMILES: COc1ncnc(OC)c1C(=O)Nc1nc2c(s1)C1(CN(CC(C)C)C1)Oc1cc(C(F)(F)F)ccc1-2] |
| 151 | 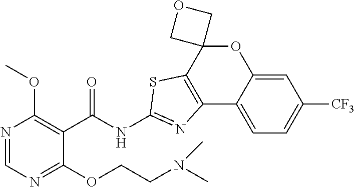 [SMILES: COc1ncnc(OCCN(C)C)c1C(=O)Nc1nc2c(s1)C1(COC1)Oc1cc(C(F)(F)F)ccc1-2] |
| 152 | 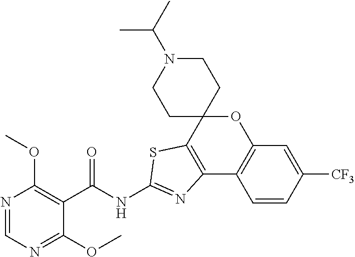 [SMILES: COc1ncnc(OC)c1C(=O)Nc1nc2c(s1)C1(CCN(C(C)C)CC1)Oc1cc(C(F)(F)F)ccc1-2] |
| 153 | 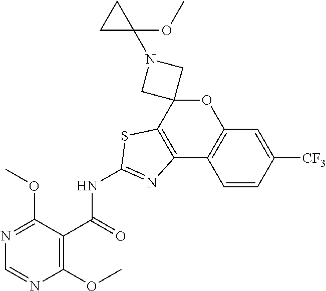 [SMILES: COc1ncnc(OC)c1C(=O)Nc1nc2c(s1)C1(CN(C3(OC)CC3)C1)Oc1cc(C(F)(F)F)ccc1-2] |
| 154 | 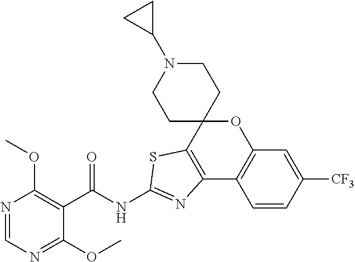 [SMILES: COc1ncnc(OC)c1C(=O)Nc1nc2c(s1)C1(CCN(C3CC3)CC1)Oc1cc(C(F)(F)F)ccc1-2] |
| 155 | 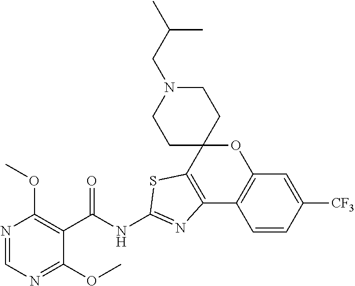 [SMILES: COc1ncnc(OC)c1C(=O)Nc1nc2c(s1)C1(CCN(CC(C)C)CC1)Oc1cc(C(F)(F)F)ccc1-2] |
| 156 | 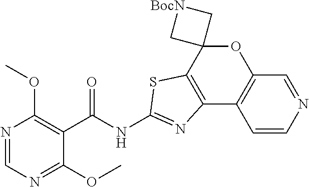 [SMILES: COc1ncnc(OC)c1C(=O)Nc1nc2c(s1)C1(CN(C(=O)OC(C)(C)C)C1)Oc1cnccc1-2] |
| 157 | 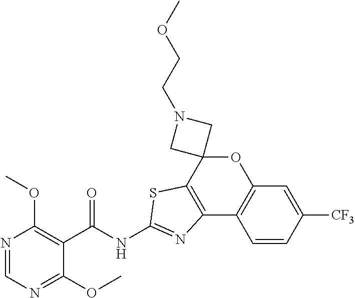 [SMILES: COCCN1CC2(C1)Oc1cc(C(F)(F)F)ccc1-c1nc(NC(=O)c3c(OC)ncnc3OC)sc12] |
| 158 | 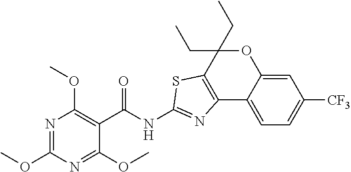 [SMILES: CCC1(CC)Oc2cc(C(F)(F)F)ccc2-c2nc(NC(=O)c3c(OC)nc(OC)nc3OC)sc21] |
| 159 | 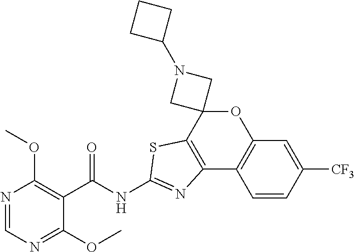 [SMILES: COc1ncnc(OC)c1C(=O)Nc1nc2c(s1)C1(CN(C3CCC3)C1)Oc1cc(C(F)(F)F)ccc1-2] |
| 160 | 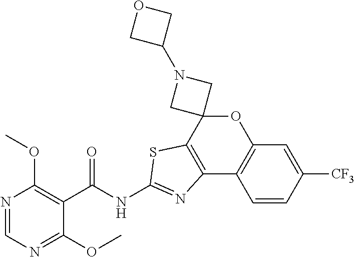 [SMILES: COc1ncnc(OC)c1C(=O)Nc1nc2c(s1)C1(CN(C3COC3)C1)Oc1cc(C(F)(F)F)ccc1-2] |
| 161 | 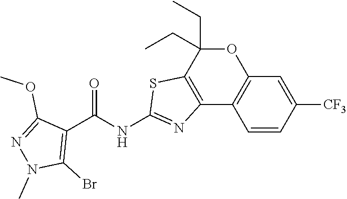 [SMILES: CCC1(CC)Oc2cc(C(F)(F)F)ccc2-c2nc(NC(=O)c3c(OC)nn(C)c3Br)sc21] |
| 162 | 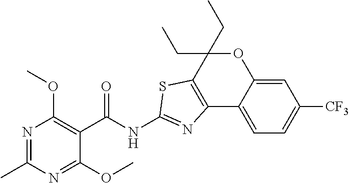 [SMILES: CCC1(CC)Oc2cc(C(F)(F)F)ccc2-c2nc(NC(=O)c3c(OC)nc(C)nc3OC)sc21] |
| 163 | 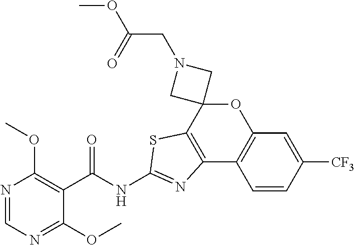 [SMILES: COC(=O)CN1CC2(C1)Oc1cc(C(F)(F)F)ccc1-c1nc(NC(=O)c3c(OC)ncnc3OC)sc12] |
| 164 | 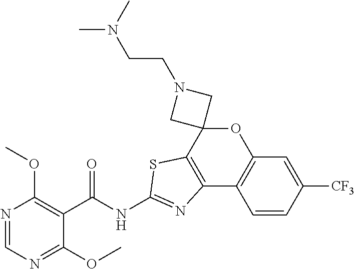 [SMILES: COc1ncnc(OC)c1C(=O)Nc1nc2c(s1)C1(CN(CCN(C)C)C1)Oc1cc(C(F)(F)F)ccc1-2] |
| 165 | 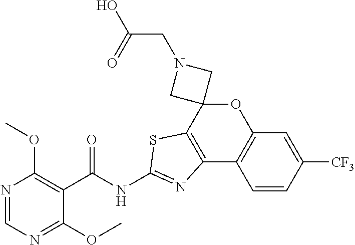 [SMILES: COc1ncnc(OC)c1C(=O)Nc1nc2c(s1)C1(CN(CC(=O)O)C1)Oc1cc(C(F)(F)F)ccc1-2] |
| 166 | 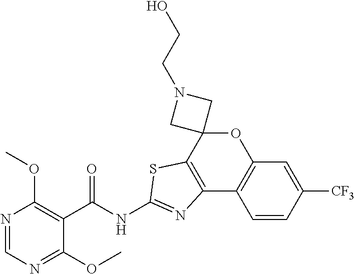 [SMILES: COc1ncnc(OC)c1C(=O)Nc1nc2c(s1)C1(CN(CCO)C1)Oc1cc(C(F)(F)F)ccc1-2] |
| 167 | 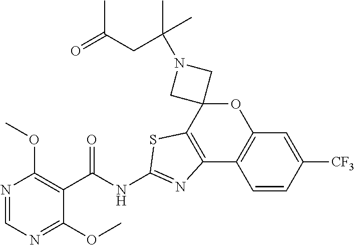 [SMILES: COc1ncnc(OC)c1C(=O)Nc1nc2c(s1)C1(CN(C(C)(C)CC(C)=O)C1)Oc1cc(C(F)(F)F)ccc1-2] |
| 168 | 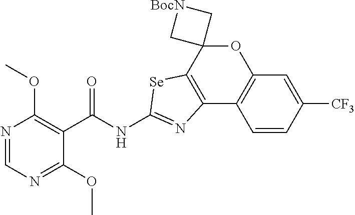 [SMILES: COc1ncnc(OC)c1C(=O)Nc1nc2c([se]1)C1(CN(C(=O)OC(C)(C)C)C1)Oc1cc(C(F)(F)F)ccc1-2] |

or a salt thereof.

594. The compound of any one of the preceding embodiments, wherein the compound is a pharmaceutically acceptable salt.

595. The compound of any one of the preceding embodiments, wherein the compound has an enantiomeric purity of about or at least about 80%.

596. The compound of any one of the preceding embodiments, wherein the compound has an enantiomeric purity of about or at least about 90%.

597. The compound of any one of the preceding embodiments, wherein the compound has an enantiomeric purity of about or at least about 95%.

598. The compound of any one of the preceding embodiments, wherein the compound has an enantiomeric purity of about or at least about 98%.

599. The compound of any one of the preceding embodiments, wherein the compound has a purity of about or at least about 90%.

600. The compound of any one of the preceding embodiments, wherein the compound has a purity of about or at least about 95%.

601. The compound of any one of the preceding embodiments, wherein the compound has a purity of about or at least about 98%.

602. The compound of any one of the preceding embodiments, wherein the compound has a purity of about or at least about 98%.

603. A pharmaceutical composition comprising a compound of any one of the preceding embodiments and a pharmaceutically acceptable carrier.

604. A method for treating a condition, disorder or disease, comprising administering to a subject suffering therefrom an effective amount of the compound or pharmaceutical composition of any one of embodiments 1-603.

605. A method for treating a condition, disorder or disease, comprising delivering to a subject suffering therefrom an effective amount of the compound or pharmaceutical composition of any one of embodiments 1-603.

606. A method for preventing a condition, disorder or disease, comprising administering to a subject susceptible thereto an effective amount of the compound or pharmaceutical composition of any one of embodiments 1-603.

607. A method for preventing a condition, disorder or disease, comprising delivering to a subject susceptible thereto an effective amount of the compound or pharmaceutical composition of any one of embodiments 1-603.

608. The method of any one of embodiments 604-607, wherein the condition, disorder or disease is associated with TRPV3.

609. The method of any one of embodiments 604-607, wherein the condition, disorder or disease is modulated by TRPV3.

610. The method of any one of embodiments 604-607, wherein the compound or pharmaceutical composition is administered in combination with one or more additional compounds or agents.

611. The method of any one of embodiments 604-607, wherein the compound or pharmaceutical composition is delivered in combination with one or more additional compounds or agents.

612. The method of any one of embodiments 604-611, wherein the condition, disorder or disease is or comprises migraine, arthralgia, cardiac pain arising from an ischemic myocardium, acute pain, chronic pain, nociceptive pain, neuropathic pain, post-operative pain, pain due to neuralgia (e.g., post-herpetic neuralgia, traumatic neuralgia, fibromyalgia, trigeminal neuralgia), pain due to diabetic neuropathy, dental pain, cancer pain, or inflammatory pain conditions (e.g. arthritis and osteoarthritis).

613. The method of any one of embodiments 604-611, wherein the condition, disorder or disease is or comprises neuropathic pain, nociceptive pain, dental pain, HIV pain, cardiac pain arising from an ischemic myocardium, pain due to migraine, arthralgia, neuropathies, neurodegeneration, retinopathy, neurotic skin disorder, stroke, urinary bladder hypersensitiveness, urinary incontinence, vulvodynia, gastrointestinal disorders such as irritable bowel syndrome, gastro-esophageal reflux disease, enteritis, ileitis, stomach-duodenal ulcer, inflammatory bowel disease, Crohn's disease, celiac disease, an inflammatory disease such as pancreatitis, a respiratory disorder such as allergic and non-allergic rhinitis, asthma or chronic obstructive pulmonary disease, irritation of skin, eye or mucous membrane, atopic dermatitis, eczema itch, fervescence, muscle spasms, emesis, dyskinesias, depression, Huntington's disease, memory deficits, restricted brain function, amyotrophic lateral sclerosis (ALS), dementia, arthritis, osteoarthritis, diabetes, obesity, urticaria, actinic keratosis, keratocanthoma, alopecia, Meniere's disease, tinnitus, hyperacusis, anxiety disorders, or benign prostate hyperplasia.

614. The method of any one of embodiments 604-611, wherein the condition, disorder or disease is or comprises atopic dermatitis, eczema, sebhorreic eczema, itch, skin inflammation, or psoriasis.

615. The method of any one of embodiments 604-611, wherein the condition, disorder or disease is acne.

616. The method of any one of embodiments 604-611, wherein the condition, disorder or disease is rosacea.

617. The method of any one of embodiments 604-611, wherein the condition, disorder or disease is pain.

618. The method of any one of embodiments 604-611, wherein the condition, disorder or disease is itch.

619. The method of any one of embodiments 604-611, wherein the condition, disorder or disease is skin inflammation.

620. The method of any one of embodiments 604-611, wherein the condition, disorder or disease comprises or is hair loss.

621. The method of any one of embodiments 604-611, wherein the condition, disorder or disease comprises or is keratoderma.

622. The method of any one of embodiments 604-621, wherein a compound or composition is administered or delivered orally.

623. The method of any one of embodiments 604-621, wherein a compound or composition is administered or delivered topically.

624. A method, comprising:
reacting a compound having the structure of formula (5):

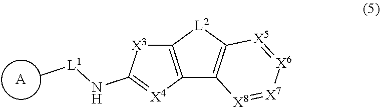

or a salt thereof to provide a compound having the structure of formula I:

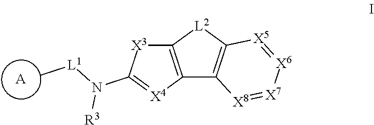

or a salt thereof, wherein each variable is independently as described in any one of the preceding embodiments.

625. A method, comprising:
reacting a compound having the structure of formula (5):

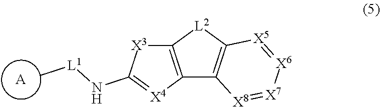

or a salt thereof with a compound having the structure of R³-LG or a salt thereof to provide a compound having the structure of formula I:

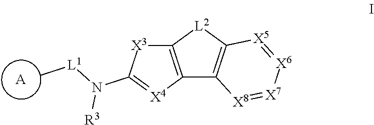

or a salt thereof, wherein each variable is independently as described in any one of the preceding embodiments.

626. A method, comprising:
reacting a compound having the structure of formula (4):

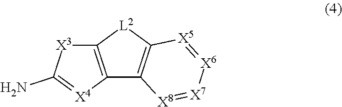

or a salt thereof to provide a compound having the structure of formula (5):

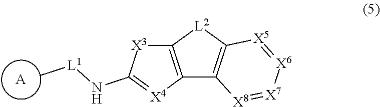

or a salt thereof, wherein each variable is independently as described in any one of the preceding embodiments.

627. A method, comprising:
reacting a compound having the structure of formula (4):

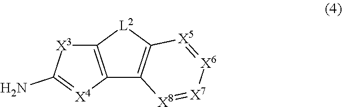

or a salt thereof with a compound having the structure of

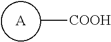

or a salt or an activated derivative thereof, or a compound having the structure of

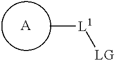

or a salt thereof, to provide a compound having the structure of formula (5):

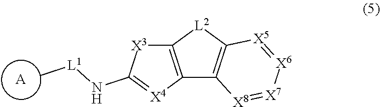

or a salt thereof, wherein each variable is independently as described in any one of the preceding embodiments.

628. The compound or method of any one of the preceding embodiments, wherein LG is halogen.

629. The compound or method of any one of the preceding embodiments, wherein LG is —Cl.

630. The compound or method of any one of the preceding embodiments, wherein LG is —Br.

631. The compound or method of any one of the preceding embodiments, wherein LG is —I.

632. The method of any one of embodiments 624-625, comprising a method of any one of embodiments 626-631.

633. A method, comprising:
reacting a compound having the structure of formula (3):

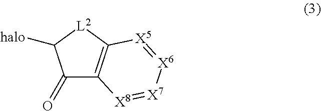

or a salt thereof to provide a compound having the structure of formula (4):

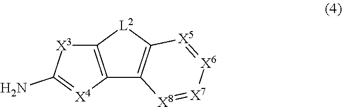

or a salt thereof, wherein each variable is independently as described in any one of the preceding embodiments.

634. A method, comprising:
reacting a compound having the structure of formula (3):

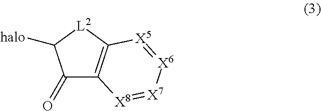

or a salt thereof with a thiourea or a salt thereof, or a selenourea or a salt thereof, to provide a compound having the structure of formula (4):

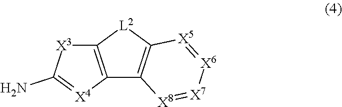

or a salt thereof, wherein each variable is independently as described in any one of the preceding embodiments.

635. The method of any one of embodiments 624-634, comprising a method of embodiment 633 or 634.

636. A method, comprising:
reacting a compound having the structure of formula (2):

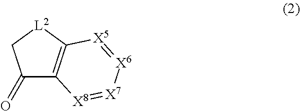

or a salt thereof to provide a compound having the structure of formula (3):

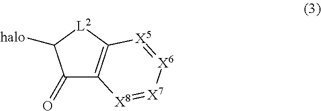

or a salt thereof, wherein each variable is independently as described in any one of the preceding embodiments.

637. The method of any one of embodiments 624-635, comprising a method of embodiment 636.

638. A method, comprising:
reacting a compound having the structure of formula (2):

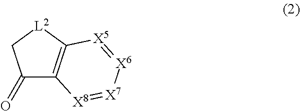

or a salt thereof to provide a compound having the structure of formula (4):

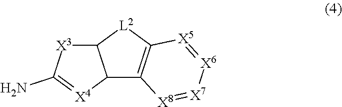

or a salt thereof, wherein each variable is independently as described in any one of the preceding embodiments.

639. The method of any one of embodiments 624-630, comprising a method of embodiment 638.

640. A method, comprising:
reacting a compound having the structure of formula (1):

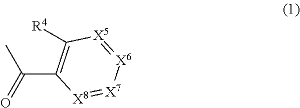

or a salt thereof to provide a compound having the structure of formula (2):

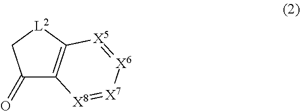

or a salt thereof, wherein each variable is independently as described in any one of the preceding embodiments.

641. A method, comprising:
reacting a compound having the structure of formula (1):

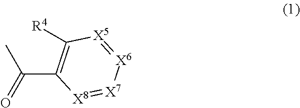

or a salt thereof with a compound having the structure of R⁴C(O)R⁵ or a salt thereof to provide a compound having the structure of formula (2):

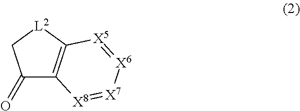

or a salt thereof, wherein each variable is independently as described in any one of the preceding embodiments.

642. The method of any one of embodiments 624-639, comprising a method of embodiment 640 or 641.

643. A method for preparing a compound of any one of embodiments 1-602, comprising a method of any one of embodiments 624-642.

644. A compound or composition prepared by a method of any one of embodiments 624-642.

645. A compound, composition, or method described in the specification.

### EXEMPLIFICATION

Certain examples of provided technologies (e.g., compounds, compositions, methods (methods of preparation, use, assessment, etc.), etc.) are described herein. Those skilled in the art reading the present disclosure appreciate that various technologies, including those described below and modifications, variants and derivatives thereof, are available for manufacturing, characterizing and/or assessing provided technologies in accordance with the present disclosure.

Certain abbreviations utilized in the Examples below:
EA=ethyl acetate;
DMF=N,N-dimethylformamide;
Dess-Martin periodinane=1,1-dihydro-1,1,1-triacetoxy-1,2-benzoiodooxol-3(1H)-one;
DCM=Dichloromethane;
THF=Tetrahydrofuran;
LAH=Lithium aluminium hydride;
DME=1,2-Dimethoxyethane;
TEA=Triethylamine;
EIPEA-Ethyldiisopropylamine;
DEAD=Diethylazodicarboxylate;
DIAD=Diisopropyl azodicarboxylate;
HATU=2-(7-Aza-1H-benzotriazole-1-yl)-1,1,3,3-tetramethyluronium hexafluorophosphate;
EDCI=N-(3-dimethylaminopropyl)-N′-ethylcarbodiimide hydrochloride;
HOBt=1-Hydroxybenzotriazole;
DIBAL-H=Diisobutylaluminium hydride;
Bn=Benzyl; DMSO=Dimethyl sulfoxide;
RT=room temperature;
DBU=1,8-Diazabicyclo[5.4.0]undec-7-ene;
NCS=N-Chlorosuccinimide;
LiHMDS=Lithium bis(trimethylsilyl) amide;
DMAP=4-Dimethylaminopyridine;
n-BuLi=n-Butyllithium;
ACN=acetonitrile;
TCFH=Chloro-N,N,N′,N′-tetramethylformamidinium Hexafluorophosphate;
NMI=1-Methylimidazole;

Certain useful materials and experimentations: Various reagents were reported and/or purchased from commercial suppliers (Sigma-Aldrich, Alfa, Across etc.) and can be used without further purification unless otherwise stated. THF can be continuously refluxed and freshly distilled from sodium and benzophenone under nitrogen, and DCM can be continuously refluxed and freshly distilled from CaH₂ under nitrogen. In some embodiments, reactions were monitored via TLC, e.g., on silica gel 60 HSGF254 percolated plates (0.15-0.2 mm SiO₂) and visualized using UV light and/or staining with a solution of DNP (12 g 2,4-dinitrofenylhydrazin, 60 mL H₂S0₄ con., 80 mL H₂O, 200 mL EtOH) and subsequent heating. In some embodiments, reactions were monitored via LCMS (e.g., Chromolith SpeedROD, RP-18e, 50×4.6 mm, mobile phase: Solvent A: CH₃CN/H₂O/HCOOH=10/90/0.05, Solvent B: CH₃CN/H₂O/HCOOH=90/10/0.05, 0.8 min @ 10% B, 2.7 min gradient (10-95% B), then 0.8 min @95% B, flow rate: 3 mL/min, temperature: 40° C.; and Chromolith SpeedROD, RP-18e, 50×4.6 mm, mobile phase: Solvent A: CH₃CN/H₂O/HCOOH=10/90/0.05, Solvent B: CH₃CN/H₂O/HCOOH=90/10/0.05, 0.8 min @ 10% B, 2.7 min gradient (10-95% B), then 0.8 min @ 95% B, flow rate: 3 mL/min, temperature: 40° C.). In some embodiments, separations were accomplished by Prep-HPLC (Column: Xbridge BEH C18 OBD column, 5 μM, 10 mm*250 mm; Mobile phase: [water (0.05% FA)-ACN]; B %: 10%- 90% over 20 min). In some embodiments, separations were accomplished by Prep-TLC (precoated silica gel 60 F 254 plates supplied by NuoTai®; visualization was accomplished under ultraviolet light (λ254 nm, λ366 nm)). In some embodiments, 1H spectra were recorded on JEOL® 400 MHz, Chemical shifts (8) are reported in ppm relative to tetramethylsilane (8=0.000 ppm) and the spectra were calibrated to the residual solvent signal of chloroform (δ=7.26 for 1H). Data for ¹H NMR spectra are reported as follows: chemical shift (multiplicity, number of hydrogens). Abbreviations are as follows: s (singlet), d (doublet), t (triplet), q (quartet), quant (quintet), m (multiple), br (broad).

A useful procedure: In some embodiments, compounds of formula (I) can be prepared using one or more steps illustrated below, wherein each variable is independently as described herein:

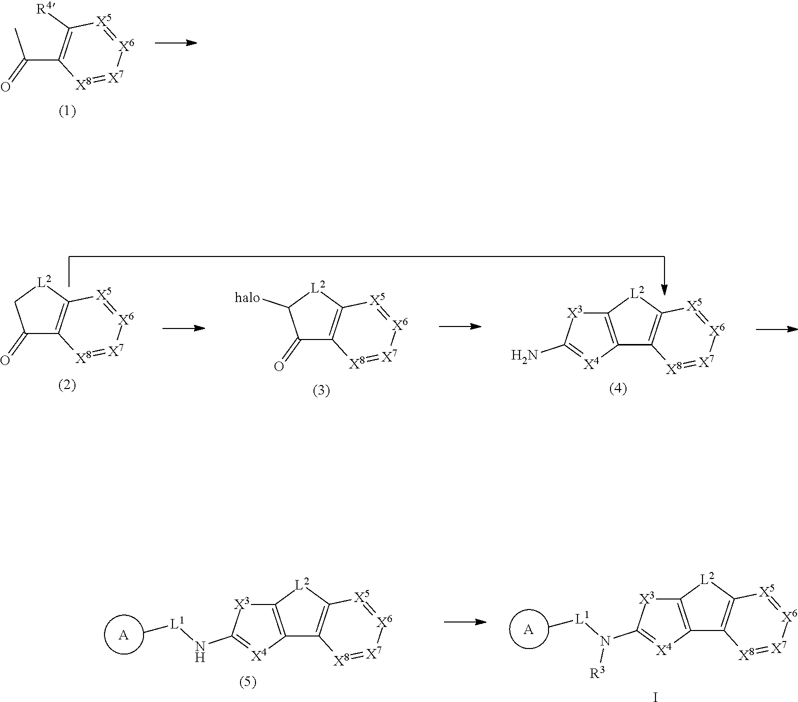

In some embodiments, a ketone compound (1) may be contacted with a ketone or aldehyde to prepare ketone (2). follow by treating with halogen (Br₂, I₂, etc.) to provide halo-ketone (3). A halo-ketone compound (3) may be contacted with a thiourea or selenourea reactant to prepare amine (4). A ketone compound (2) may be contacted with a halogen (e.g., Br₂, I₂, etc.) reactant to prepare amine (4) in a one-pot reaction. In some embodiments, halo is chloro, bromo, or iodo. A cyclization reaction may be carried out in a solvent such as an alcohol (e.g., methanol, ethanol, etc.), at room temperature or at an elevated temperature (e.g., about 40° C. or above, such as between about 60° C. and about 70° C.), optionally with stirring.

An amine (4) may be contacted with a suitable reactant (e.g., as described in an Example) to provide compound (5). In some embodiments, a coupling reaction may be carried out in a solvent such as methylene chloride, and/or THF, and/or DMF, at room temperature or at an elevated temperature (e.g., about 40° C. or above, such as between about 60° C. and about 70° C.) optionally with stirring. In some embodiments, N-alkylation of a compound (5) provides a compound having the structure of formula (I) or a salt thereof, e.g., by treating with R³I or R³Br in a suitable solvent, e.g., DMF, DMSO or THF.

Certain preparations of certain compounds are presented below as examples.

### Example 1: 4,6-dimethoxy-N—(7-(trifluoromethyl)-4H-chromeno[4,3-d]thiazol-2-yl)pyrimidine-5-carboxamide

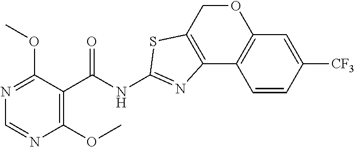 [SMILES: COc1ncnc(OC)c1C(=O)Nc1nc2c(s1)COc1cc(C(F)(F)F)ccc1-2]

### Example 1A: 7-(trifluoromethyl)-4H-chromeno[4,3-d]thiazol-2-amine

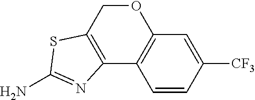 [SMILES: Nc1nc2c(s1)COc1cc(C(F)(F)F)ccc1-2]

To a stirred solution of 7-(trifluoromethyl) chroman-4-one (50 mg, 0.23 mmol, 1.00 equiv.) in EtOH (1 mL) was added thiourea (53 mg, 0.69 mmol, 3.00 equiv.) and iodine (71 mg, 0.28 mmol, 1.20 equiv.) at room temperature. The resulting mixture was stirred for overnight at 100° C. Then it was cooled to room temperature and the resulting mixture was extracted with EA (2×10 mL). The combined organic layers were washed with 1N NaOH (1×3 mL) and brine (1×10 mL), dried over anhydrous Na₂SO₄. After filtration, the filtrate was concentrated under reduced pressure. The crude product was purified by prep-TLC (PE:EA=4:1) to afford the title compound (13 mg, 21%) as a yellow solid. ESI-MS m/z=272.93 [M+H]⁺; Calculated MW: 272.02.

### Example 1B: 4,6-dimethoxy-N—(7-(trifluoromethyl)-4H-chromeno[4,3-d]thiazol-2-yl)pyrimidine-5-carboxamide

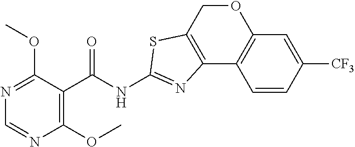 [SMILES: COc1ncnc(OC)c1C(=O)Nc1nc2c(s1)COc1cc(C(F)(F)F)ccc1-2]

To a suspension of 4,6-dimethoxypyrimidine-5-carboxylic acid (11 mg, 0.057 mmol, 1.20 equiv.) in CH₂Cl₂ (0.5 mL) and catalytic amount of DMF, oxalyl chloride (12 mg, 0.095 mmol, 2.0 equiv.) was added dropwise at 0° C., and the mixture was stirred for 3 h at room temperature. Then the reaction mixture was concentrated in vacuo and dissolved in CH₂Cl₂ (0.5 mL), 7-(trifluoromethyl)-4H-chromeno[4,3-d]thiazol-2-amine (13 mg, 0.048 mmol, 1.0 equiv.), TEA (84 mg, 0.191 mmol, 4.0 equiv.) and DMAP (0.58 mg, 0.0048 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=3:1) to afford the title compound (10 mg, 62%) as a yellow solid. ESI-MS m/z=438.98 [M+H]⁺; Calculated MW: 438.06. ¹H NMR (400 MHz, Chloroform-d) δ 11.09 (s, 1H), 8.32 (s, 1H), 7.58 (dd, J=8.2, 1.1 Hz, 1H), 7.16 (d, J=6.6 Hz, 2H), 5.50 (s, 2H), 3.99 (s, 6H).

### Example 2: N—(4,5-dihydronaphtho[1,2-d]thiazol-2-yl)-4,6-dimethoxypyrimidine-5-carboxamide

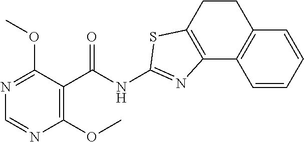 [SMILES: COc1ncnc(OC)c1C(=O)Nc1nc2c(s1)CCc1ccccc1-2]

### Example 2A: 4,5-dihydronaphtho[1,2-d]thiazol-2-amine

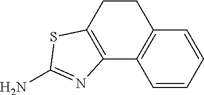 [SMILES: Nc1nc2c(s1)CCc1ccccc1-2]

To a stirred solution of 3,4-dihydronaphthalen-1 (2H)-one (100 mg, 0.68 mmol, 1.00 equiv.) in EtOH (1 mL) was added thiourea (156 mg, 2.05 mmol, 3.00 equiv.) and iodine (209 mg, 0.82 mmol, 1.20 equiv.) at room temperature. The resulting mixture was stirred for overnight at 100° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (100 mg, 72%) as a purple solid. ¹H NMR (400 MHz, Chloroform-d) δ 7.69-7.64 (m, 1H), 7.24-7.21 (m, 1H), 7.18-7.10 (m, 2H), 4.92 (s, 2H), 3.01 (dd, J=8.8, 6.8 Hz, 2H), 2.91-2.77 (m, 2H).

### Example 2B: N—(4,5-dihydronaphtho[1,2-d]thiazol-2-yl)-4,6-dimethoxypyrimidine-5-carboxamide

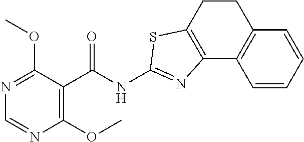 [SMILES: COc1ncnc(OC)c1C(=O)Nc1nc2c(s1)CCc1ccccc1-2]

To a suspension of 4,6-dimethoxypyrimidine-5-carboxylic acid (27 mg, 0.148 mmol, 1.20 equiv.) in CH₂Cl₂ (0.5 mL) and catalytic amount of DMF, oxalyl chloride (32 mg, 0.248 mmol, 2.0 equiv.) was added dropwise at 0° C., and the mixture was stirred for 3 h at room temperature. Then the reaction mixture was concentrated in vacuo and dissolved in CH₂Cl₂ (0.5 mL), 4,5-dihydronaphtho[1,2-d]thiazol-2-amine (25 mg, 0.124 mmol, 1.0 equiv.), TEA (50 mg, 0.495 mmol, 4.0 equiv.) and DMAP (1.5 mg, 0.012 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (4.8 mg, 11%) as a yellow solid. ESI-MS m/z=369.06 [M+H]⁺; Calculated MW: 368.09. ¹H NMR (400 MHz, Chloroform-d) δ 11.80 (s, 1H), 8.13 (s, 1H), 7.57-7.48 (m, 1H), 7.19-7.12 (m, 3H), 3.86 (s, 6H), 3.08-2.90 (m, 4H).

### Example 3: N—(8-chloro-4,5-dihydronaphtho[1,2-d]thiazol-2-yl)-4,6-dimethoxypyrimidine-5-carboxamide

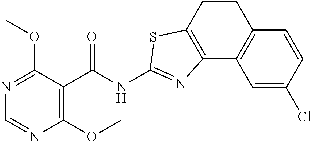 [SMILES: COc1ncnc(OC)c1C(=O)Nc1nc2c(s1)CCc1ccc(Cl)cc1-2]

### Example 3A: 8-chloro-4,5-dihydronaphtho[1,2-d]thiazol-2-amine

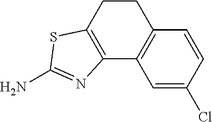 [SMILES: Nc1nc2c(s1)CCc1ccc(Cl)cc1-2]

To a stirred solution of 7-chloro-3,4-dihydronaphthalen-1 (2H)-one (100 mg, 0.56 mmol, 1.00 equiv.) in EtOH (1 mL) was added thiourea (127 mg, 1.67 mmol, 3.00 equiv.) and iodine (169 mg, 0.67 mmol, 1.20 equiv.) at room temperature. The resulting mixture was stirred for overnight at 100° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (100 mg, 31%) as a yellow solid. ¹H NMR (400 MHz, Chloroform-d) δ 7.66 (d, J=1.8 Hz, 1H), 7.13-7.04 (m, 2H), 4.87 (s, 2H), 2.97 (dd, J=9.1, 6.9 Hz, 2H), 2.88-2.79 (m, 2H).

### Example 3B: N—(8-chloro-4,5-dihydronaphtho[1,2-d]thiazol-2-yl)-4,6-dimethoxypyrimidine-5-carboxamide

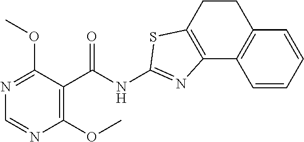 [SMILES: COc1ncnc(OC)c1C(=O)Nc1nc2c(s1)CCc1ccccc1-2]

To a suspension of 4,6-dimethoxypyrimidine-5-carboxylic acid (24 mg, 0.127 mmol, 1.20 equiv.) in CH₂Cl₂ (0.5 mL) and catalytic amount of DMF, oxalyl chloride (27 mg, 0.212 mmol, 2.0 equiv.) was added dropwise at 0° C., and the mixture was stirred for 3 h at room temperature. Then the reaction mixture was concentrated in vacuo and dissolved in CH₂Cl₂ (0.5 mL), 8-chloro-4,5-dihydronaphtho[1,2-d]thiazol-2-amine (25 mg, 0.106 mmol, 1.0 equiv.), TEA (43 mg, 0.424 mmol, 4.0 equiv.) and DMAP (1.3 mg, 0.011 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (7.7 mg, 18%) as a yellow solid. ESI-MS m/z=403.04 [M+H]⁺; Calculated MW: 402.05. ¹H NMR (400 MHz, Chloroform-d) § 12.00 (s, 1H), 8.09 (s, 1H), 7.45 (d, J=1.8 Hz, 1H), 7.11 (t, J=1.6 Hz, 2H), 3.83 (s, 6H), 3.03-2.91 (m, 4H).

### Example 4: N—(5,6-dihydro-4H-benzo[6,7] cyclohepta[1,2-d]thiazol-2-yl)-4,6-dimethoxypyrimidine-5-carboxamide

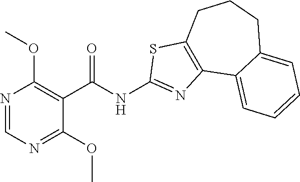 [SMILES: COc1ncnc(OC)c1C(=O)Nc1nc2c(s1)CCCc1ccccc1-2]

### Example 4A: 5,6-dihydro-4H-benzo[6,7] cyclohepta[1,2-d]thiazol-2-amine

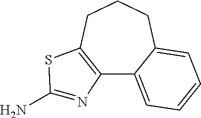 [SMILES: Nc1nc2c(s1)CCCc1ccccc1-2]

To a stirred solution of 6,7,8,9-tetrahydro-5H-benzo[7]annulen-5-one (100 mg, 0.625 mmol, 1.00 equiv.) in EtOH (1 mL) was added thiourea (143 mg, 1.875 mmol, 3.00 equiv.) and iodine (191 mg, 0.75 mmol, 1.20 equiv.) at room temperature. The resulting mixture was stirred for overnight at 100° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (47 mg, 35%) as a yellow solid. ¹H NMR (400 MHz, Chloroform-d) δ 7.89 (dd, J=7.6, 1.3 Hz, 1H), 7.25-7.22 (m, 1H), 7.19-7.11 (m, 2H), 4.85 (s, 2H), 2.84-2.78 (m, 4H), 2.12 (dtd, J=11.7, 7.0, 6.4, 2.7 Hz, 2H).

### Example 4B: N—(5,6-dihydro-4H-benzo[6,7] cyclohepta[1,2-d]thiazol-2-yl)-4,6-dimethoxypyrimidine-5-carboxamide

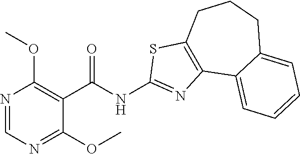 [SMILES: COc1ncnc(OC)c1C(=O)Nc1nc2c(s1)CCCc1ccccc1-2]

To a suspension of 4,6-dimethoxypyrimidine-5-carboxylic acid (26 mg, 0.139 mmol, 1.20 equiv.) in CH₂Cl₂ (0.5 mL) and catalytic amount of DMF, oxalyl chloride (29 mg, 0.231 mmol, 2.0 equiv.) was added dropwise at 0° C., and the mixture was stirred for 3 h at room temperature. Then the reaction mixture was concentrated in vacuo and dissolved in CH₂Cl₂ (0.5 mL), 5,6-dihydro-4H-benzo[6,7] cyclohepta[1,2-d]thiazol-2-amine (25 mg, 0.116 mmol, 1.0 equiv.), TEA (47 mg, 0.463 mmol, 4.0 equiv.) and DMAP (1.4 mg, 0.012 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (9.8 mg, 22%) as a yellow solid. ESI-MS m/z=383.09 [M+H]⁺; Calculated MW: 382.11. ¹H NMR (400 MHz, Chloroform-d) δ 12.45 (s, 1H), 8.14 (s, 1H), 7.77-7.65 (m, 1H), 7.17-7.08 (m, 3H), 3.74 (s, 6H), 2.94 (t, J=7.1 Hz, 2H), 2.82-2.69 (m, 2H), 2.25-2.09 (m, 2H).

### Example 5: N—(8-chloro-4H-thiochromeno[4,3-d]thiazol-2-yl)-4,6-dimethoxypyrimidine-5-carboxamide

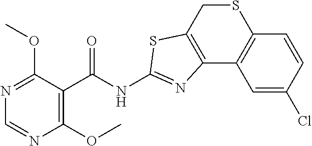 [SMILES: COc1ncnc(OC)c1C(=O)Nc1nc2c(s1)CSc1ccc(Cl)cc1-2]

### Example 5A: 8-chloro-4H-thiochromeno[4,3-d]thiazol-2-amine

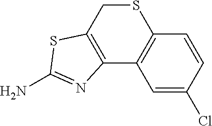 [SMILES: Nc1nc2c(s1)CSc1ccc(Cl)cc1-2]

To a stirred solution of 6-chlorothiochroman-4-one (100 mg, 0.505 mmol, 1.00 equiv.) in EtOH (1 mL) was added thiourea (115 mg, 1.515 mmol, 3.00 equiv.) and iodine (154 mg, 0.606 mmol, 1.20 equiv.) at room temperature. The resulting mixture was stirred for overnight at 100° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (47 mg, 16%) as a yellow solid. ¹H NMR (400 MHz, Chloroform-d) δ 7.78 (d, J=2.3 Hz, 1H), 7.18 (d, J=8.3 Hz, 1H), 7.07 (dd, J=8.3, 2.3 Hz, 1H), 4.92 (s, 2H), 3.99 (s, 2H).

### Example 5B: N—(8-chloro-4H-thiochromeno[4,3-d]thiazol-2-yl)-4,6-dimethoxypyrimidine-5-carboxamide

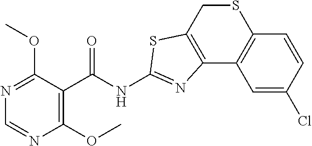 [SMILES: COc1ncnc(OC)c1C(=O)Nc1nc2c(s1)CSc1ccc(Cl)cc1-2]

To a suspension of 4,6-dimethoxypyrimidine-5-carboxylic acid (17 mg, 0.094 mmol, 1.20 equiv.) in CH₂Cl₂ (0.5 mL) and catalytic amount of DMF, oxalyl chloride (20 mg, 0.157 mmol, 2.0 equiv.) was added dropwise at 0° C., and the mixture was stirred for 3 h at room temperature. Then the reaction mixture was concentrated in vacuo and dissolved in CH₂Cl₂ (0.5 mL), 8-chloro-4H-thiochromeno[4,3-d]thiazol-2-amine (20 mg, 0.079 mmol, 1.0 equiv.), TEA (32 mg, 0.315 mmol, 4.0 equiv.) and DMAP (1 mg, 0.0079 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (9.8 mg, 8%) as a yellow solid. ESI-MS m/z=420.97 [M+H]⁺; Calculated MW: 420.01. ¹H NMR (400 MHz, Chloroform-d) δ 8.45 (s, 1H), 7.82 (d, J=2.3 Hz, 1H), 7.12 (dd, J=8.3, 2.3 Hz, 1H), 4.13 (s, 2H), 4.10 (s, 6H).

### Example 6: N—(7-bromo-4H-chromeno[4,3-d]thiazol-2-yl)-4,6-dimethoxypyrimidine-5-carboxamide

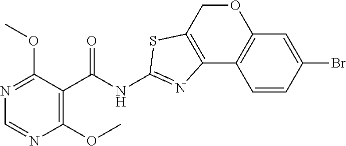 [SMILES: COc1ncnc(OC)c1C(=O)Nc1nc2c(s1)COc1cc(Br)ccc1-2]

### Example 6A: 7-bromo-4H-chromeno[4,3-d]thiazol-2-amine

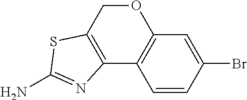 [SMILES: Nc1nc2c(s1)COc1cc(Br)ccc1-2]

To a stirred solution of 6-chlorothiochroman-4-one (100 mg, 0.442 mmol, 1.00 equiv.) in EtOH (1 mL) was added thiourea (101 mg, 1.327 mmol, 3.00 equiv.) and iodine (134 mg, 0.529 mmol, 1.20 equiv.) at room temperature. The resulting mixture was stirred for overnight at 100° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (20 mg, 16%) as a yellow solid. ESI-MS m/z=282.92 [M+H]⁺; Calculated MW: 281.94.

### Example 6B: N—(7-bromo-4H-chromeno[4,3-d]thiazol-2-yl)-4,6-dimethoxypyrimidine-5-carboxamide

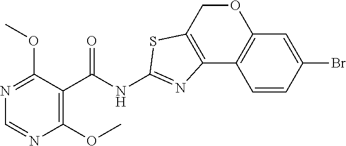 [SMILES: COc1ncnc(OC)c1C(=O)Nc1nc2c(s1)COc1cc(Br)ccc1-2]

To a stirred solution of 7-bromo-4H-chromeno[4,3-d]thiazol-2-amine (5 mg, 0.035 mmol, 1.00 equiv.), 4,6-dimethoxypyrimidine-5-carboxylic acid (16 mg, 0.089 mmol, 2.50 equiv.) and NMI (15 mg, 0.177 mmol, 5.00 equiv.) in ACN (0.2 mL) and DMF (0.2 mL) was added TCFH (25 mg, 0.089 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE:EA=4:1) to afford the title compound (3.3 mg, 21%) as a yellow solid. ESI-MS m/z=448.97 [M+H]⁺; Calculated MW: 447.98.

¹H NMR (400 MHz, Chloroform-d) δ 10.87 (s, 1H), 8.37 (s, 1H), 7.39 (d, J=8.1 Hz, 1H), 7.09-7.03 (m, 2H), 5.44 (s, 2H), 4.02 (s, 6H).

### Example 7: N—(5-chloro-8H-indeno[1,2-d]thiazol-2-yl)-4,6-dimethoxypyrimidine-5-carboxamide

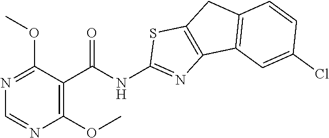 [SMILES: COc1ncnc(OC)c1C(=O)Nc1nc2c(s1)Cc1ccc(Cl)cc1-2]

### Example 7A: 5-chloro-8H-indeno[1,2-d]thiazol-2-amine

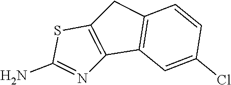 [SMILES: Nc1nc2c(s1)Cc1ccc(Cl)cc1-2]

To a stirred solution of 6-chloro-2,3-dihydro-1H-inden-1-one (100 mg, 0.602 mmol, 1.00 equiv.) in EtOH (1 mL) was added thiourea (137 mg, 1.807 mmol, 3.00 equiv.) and iodine (184 mg, 0.723 mmol, 1.20 equiv.) at room temperature. The resulting mixture was stirred for overnight at 100° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (25 mg, 19%) as a yellow solid. ESI-MS m/z=222.96 [M+H]⁺; Calculated MW: 222.00.

### Example 7B: N—(5-chloro-8H-indeno[1,2-d]thiazol-2-yl)-4,6-dimethoxypyrimidine-5-carboxamide

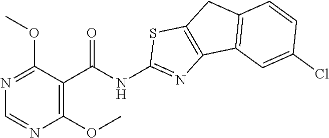 [SMILES: COc1ncnc(OC)c1C(=O)Nc1nc2c(s1)Cc1ccc(Cl)cc1-2]

To a stirred solution of 5-chloro-8H-indeno[1,2-d]thiazol-2-amine (15 mg, 0.068 mmol, 1.00 equiv.), 4,6-dimethoxypyrimidine-5-carboxylic acid (31 mg, 0.169 mmol, 2.50 equiv.) and NMI (28 mg, 0.338 mmol, 5.00 equiv.) in ACN (0.2 mL) and DMF (0.2 mL) was added TCFH (48 mg, 0.169 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE:EA=4:1) to afford the title compound (5.1 mg, 19%) as a yellow solid. ESI-MS m/z=389.00 [M+H]⁺; Calculated MW: 388.04. ¹H NMR (400 MHz, Chloroform-d) δ 10.99 (s, 1H), 8.28 (s, 1H), 7.45-7.36 (m, 2H), 7.19 (dd, J=8.0, 2.0 Hz, 1H), 4.02 (s, 6H), 3.81 (d, J=0.8 Hz, 2H).

### Example 8: N—(4,4-dimethyl-7-(trifluoromethyl)-4H-chromeno[4,3-d]thiazol-2-yl)-4,6-dimethoxypyrimidine-5-carboxamide

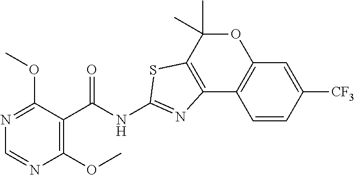 [SMILES: COc1ncnc(OC)c1C(=O)Nc1nc2c(s1)C(C)(C)Oc1cc(C(F)(F)F)ccc1-2]

### Example 8A: 1-(2-hydroxy-4-(trifluoromethyl)phenyl)ethan-1-one

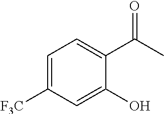

To a stirred solution of 2-hydroxy-4-(trifluoromethyl)benzoic acid (1.00 g, 4.85 mmol, 1.00 equiv.) in THF (10.00 mL) was added MeLi (9.1 mL, 14.56 mmol, 3.00 equiv., 1.6 M in diethoxymethane) in portions at 0° C. The resulting mixture was stirred for 4 h at room temperature under argon atmosphere. The resulting mixture was adjusted pH to 5 with HCl (1M), extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (10:1) to afford the title compound (780 mg, 78% yield) as a colorless liquid. ¹H NMR (400 MHz, Chloroform-d) δ 12.27 (s, 1H), 7.87-7.81 (m, 1H), 7.25-7.23 (m, 1H), 7.13 (ddd, J=8.3, 1.8, 0.7 Hz, 1H), 2.67 (s, 3H).

### Example 8B: 2,2-dimethyl-7-(trifluoromethyl) chroman-4-one

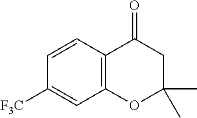 [SMILES: CC1(C)CC(=O)c2ccc(C(F)(F)F)cc2O1]

To a stirred solution of 1-(2-hydroxy-4-(trifluoromethyl)phenyl)ethan-1-one (130.0 mg, 0.637 mmol, 1.00 equiv.) and propan-2-one (37. mg, 0.637 mmol, 1.00 equiv.) in MeOH (2.00 mL) was added pyrrolidine (91.0 mg, 1.27 mmol, 2.00 equiv.) in portions at 0° C. The resulting mixture was stirred for 16 h at room temperature under argon atmosphere. The reaction was diluted with water (30 mL), extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (10:1)) to afford the title compound (115 mg, 74% yield) as a light yellow oil. ¹H NMR (400 MHz, Chloroform-d) δ 7.93 (dp, J=7.9, 1.0 Hz, 1H), 7.21-7.16 (m, 2H), 2.75 (s, 2H), 1.46 (s, 6H).

### Example 8C: 4,4-dimethyl-7-(trifluoromethyl)-4H-chromeno[4,3-d]thiazol-2-amine

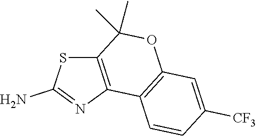 [SMILES: CC1(C)Oc2cc(C(F)(F)F)ccc2-c2nc(N)sc21]

Into a 10 mL round-bottom flask were added 2,2-dimethyl-7-(trifluoromethyl) chroman-4-one (115 mg, 0.471 mmol, 1.00 equiv.), EtOH (2 mL), thiourea (107 mg, 1.41 mmol, 3.00 equiv.), and I₂ (144 mg, 0.565 mmol, 1.20 equiv.) at room temperature. The resulting mixture was stirred for 16 h at 100° C. under argon atmosphere. The resulting mixture was cooled down to room temperature, adjusted pH to 9 with NaOH (1M), extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (15 mg, 10% yield) as a light yellow solid. ESI-MS m/z=301.03 [M+H]⁺; Calculated MW: 300.05

### Example 8D: N—(4,4-dimethyl-7-(trifluoromethyl)-4H-chromeno[4,3-d]thiazol-2-yl)-4,6-dimethoxypyrimidine-5-carboxamide

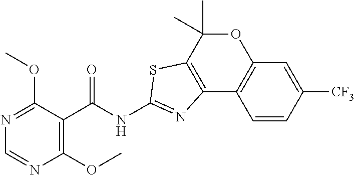 [SMILES: COc1ncnc(OC)c1C(=O)Nc1nc2c(s1)C(C)(C)Oc1cc(C(F)(F)F)ccc1-2]

To a stirred solution of 4,6-dimethoxypyrimidine-5-carboxylic acid (10.0 mg, 0.055 mmol, 1.10 equiv.) in DCM (1.00 mL) was added (COCl)₂ (14.0 mg, 0.011 mmol, 2.20 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL), 4,4-dimethyl-7-(trifluoromethyl)-4H-chromeno[4,3-d]thiazol-2-amine (15.0 mg, 0.050 mmol, 1.00 equiv.), TEA (20.0 mg, 0.200 mmol, 4.00 equiv.) and DMAP (6.0 mg, 0.050 mmol, 1.00 equiv.) in DCM (1.00 mL) was added in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc 3:1) to afford the title compound (5.5 mg, 23% yield) as an off-white solid. ESI-MS m/z=467.09 [M+H]⁺; Calculated MW: 466.09; ¹H NMR (400 MHz, Chloroform-d) δ 11.35 (s, 1H), 8.26 (s, 1H), 7.52 (dd, J=7.9, 1.0 Hz, 1H), 7.16-7.07 (m, 2H), 3.94 (s, 6H), 1.72 (s, 6H).

### Example 9: 4,6-dimethoxy-N—(4-oxo-7-(trifluoromethyl)-4H-chromeno[4,3-d]thiazol-2-yl)pyrimidine-5-carboxamide

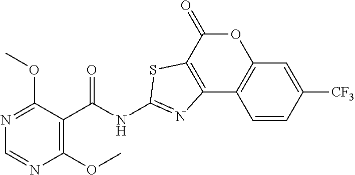 [SMILES: COc1ncnc(OC)c1C(=O)Nc1nc2c(s1)c(=O)oc1cc(C(F)(F)F)ccc12]

### Example 9A: ethyl 3-(2-hydroxy-4-(trifluoromethyl)phenyl)-3-oxopropanoate

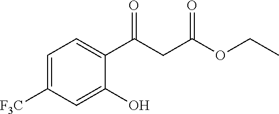 [SMILES: CCOC(=O)CC(=O)c1ccc(C(F)(F)F)cc1O]

To a stirred solution of NaH (330.0 mg, 7.84 mmol, 4.00 equiv.) in diethyl carbonate (1.50 mL) was added 1-(2-hydroxy-4-(trifluoromethyl)phenyl)ethan-1-one (400.0 mg, 1.96 mmol, 1.00 equiv.) in diethyl carbonate (1.50 mL) dropwise at 0° C. The resulting mixture was stirred for 16 h at 80° C. under argon atmosphere. The reaction was diluted with water (50 mL), adjusted pH to 5 with 1 M HCl(aq.), extracted with EtOAc (3×20 mL). The combined organic layers were washed with brine (2×20 mL), dried over anhydrous Na₂SO₄. After filtration, the filtrate was concentrated under reduced pressure. The residue was purified by silica gel chromatography, eluted with PE/EtOAc (10:1) to afford the title compound (370 mg, 68% yield) as a light yellow oil. ¹H NMR (400 MHz, Chloroform-d) δ 11.88 (s, 1H), 7.83-7.76 (m, 1H), 7.27 (dt, J=1.9, 0.6 Hz, 1H), 7.15 (ddd, J=8.4, 1.8, 0.7 Hz, 1H), 4.21 (dq, J=12.3, 7.2 Hz, 2H), 4.02 (s, 2H), 1.27 (td, J=7.2, 1.9 Hz, 3H).

### Example 9B: 7-(trifluoromethyl) chromane-2,4-dione

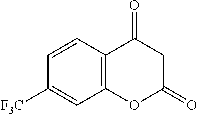 [SMILES: O=C1CC(=O)c2ccc(C(F)(F)F)cc2O1]

To a stirred solution of ethyl 3-(2-hydroxy-4-(trifluoromethyl)phenyl)-3-oxopropanoate (270.0 mg, 0.978 mmol, 1.00 equiv.) in EtOH (5 mL) was added NaOH (78.0 mg, 1.956 mmol, 2.00 equiv.) in portions at 0° C. The resulting mixture was stirred for 3 h at 80° C. under argon atmosphere. The reaction was cooled down to room temperature, diluted with water (40 mL), adjusted pH to 5 with 1 M HCl(aq.), extracted with EtOAc (3×20 mL). The combined organic layers were washed with brine (2×20 mL), dried over anhydrous Na₂SO₄. After filtration, the filtrate was concentrated under reduced pressure. This resulted in the title compound (180 mg, crude) as a light yellow solid. ¹H NMR (400 MHz, DMSO— d₆) δ 7.98 (d, J=8.2 Hz, 1H), 7.76 (d, J=1.7 Hz, 1H), 7.65 (dd, J=8.5, 1.7 Hz, 1H), 5.66 (s, 1H).

### Example 9C: 2-amino-7-(trifluoromethyl)-4H-chromeno[4,3-d]thiazol-4-one

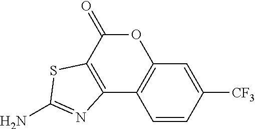 [SMILES: Nc1nc2c(s1)c(=O)oc1cc(C(F)(F)F)ccc12]

Into a 10 mL round-bottom flask were added 7-(trifluoromethyl) chromane-2,4-dione (180 mg, 0.783 mmol, 1.00 equiv.), EtOH (2 mL), thiourea (180 mg, 2.34 mmol, 3.00 equiv.), and I₂ (240 mg, 0.939 mmol, 1.20 equiv.) at room temperature. The resulting mixture was stirred for 16 h at 80° C. under argon atmosphere. The resulting mixture was cooled down to room temperature, adjusted pH to 9 with NaOH (1M), extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (15 mg, 6.6% yield) as a light yellow solid.

### Example 9D: 4,6-dimethoxy-N—(4-oxo-7-(trifluoromethyl)-4H-chromeno[4,3-d]thiazol-2-yl)pyrimidine-5-carboxamide

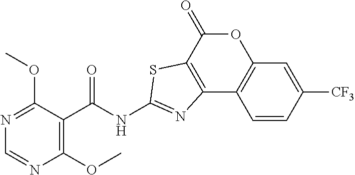 [SMILES: COc1ncnc(OC)c1C(=O)Nc1nc2c(s1)c(=O)oc1cc(C(F)(F)F)ccc12]

To a stirred solution of 4,6-dimethoxypyrimidine-5-carboxylic acid (12.0 mg, 0.063 mmol, 1.20 equiv.) in DCM (1.00 mL) was added (COCl)₂ (16.0 mg, 0.125 mmol, 2.40 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL). 2-amino-7-(trifluoromethyl)-4H-chromeno[4,3-d]thiazol-4-one (15.0 mg, 0.052 mmol, 1.00 equiv.), TEA (22.0 mg, 0.209 mmol, 4.00 equiv.) and DMAP (10.0 mg, 0.024 mmol, 0.50 equiv.) in DCM (1.00 mL) was added in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc=3:1) to afford the title compound (1.8 mg, 7.6% yield) as an off-white solid. ESI-MS m/z=453.06 [M+H]⁺; Calculated MW: 452.04; ¹H NMR (400 MHz, Chloroform-d) δ 10.60 (s, 1H), 8.55 (s, 1H), 8.24 (ddd, J=7.9, 1.2, 0.6 Hz, 1H), 7.70 (dt, J=1.7, 0.6 Hz, 1H), 7.61 (ddt, J=8.2, 1.7, 0.7 Hz, 1H), 4.20 (s, 6H).

### Example 10: N—(7-fluoro-4H-chromeno[4,3-d]thiazol-2-yl)-4,6-dimethoxypyrimidine-5-carboxamide

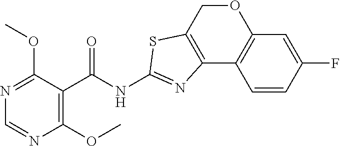 [SMILES: COc1ncnc(OC)c1C(=O)Nc1nc2c(s1)COc1cc(F)ccc1-2]

### Example 10A: 7-fluoro-4H-chromeno[4,3-d]thiazol-2-amine

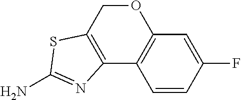 [SMILES: Nc1nc2c(s1)COc1cc(F)ccc1-2]

To a stirred solution of 7-fluorochroman-4-one (100 mg, 0.301 mmol, 1.00 equiv.) in EtOH (1 mL) was added thiourea (46 mg, 0.602 mmol, 2.00 equiv.), TEA (3 mg, 0.030 mmol, 0.10 equiv.) and iodine (92 mg, 0.361 mmol, 1.20 equiv.) at room temperature. The resulting mixture was stirred for overnight at 80° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (15 mg, 22%) as a yellow solid. ¹H NMR (400 MHz, Chloroform-d) δ 7.50 (dd, J=8.5, 6.5 Hz, 1H), 6.73-6.56 (m, 2H), 5.32 (s, 2H), 5.11-4.98 (m, 2H).

### Example 10B: N—(7-fluoro-4H-chromeno[4,3-d]thiazol-2-yl)-4,6-dimethoxypyrimidine-5-carboxamide

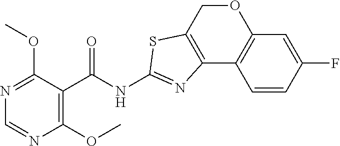 [SMILES: COc1ncnc(OC)c1C(=O)Nc1nc2c(s1)COc1cc(F)ccc1-2]

To a stirred solution of 7-fluoro-4H-chromeno[4,3-d]thiazol-2-amine (15 mg, 0.068 mmol, 1.00 equiv.), 4,6-dimethoxypyrimidine-5-carboxylic acid (31 mg, 0.169 mmol, 2.50 equiv.) and NMI (28 mg, 0.338 mmol, 5.00 equiv.) in ACN (0.2 mL) and DMF (0.2 mL) was added TCFH (48 mg, 0.169 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE:EA=4:1) to afford the title compound (3.9 mg, 15%) as a yellow solid. ESI-MS m/z=389.11 [M+H]⁺; Calculated MW: 388.06. ¹H NMR (400 MHz, Chloroform-d) δ 10.84 (s, 1H), 8.38 (s, 1H), 7.54-7.45 (m, 1H), 6.70-6.60 (m, 2H), 5.45 (s, 2H), 4.03 (d, J=1.2 Hz, 6H).

### Example 11: 4,6-dimethoxy-N—(7-methoxy-4H-chromeno[4,3-d]thiazol-2-yl)pyrimidine-5-carboxamide

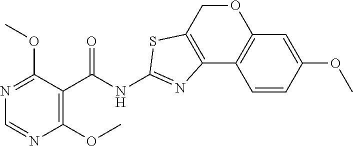

### Example 11A: 7-methoxy-4H-chromeno[4,3-d]thiazol-2-amine

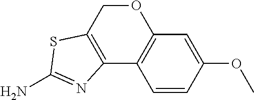 [SMILES: COc1ccc2c(c1)OCc1sc(N)nc1-2]

To a stirred solution of 7-methoxychroman-4-one (25 mg, 0.140 mmol, 1.00 equiv.) in EtOH (1 mL) was added thiourea (21 mg, 0.281 mmol, 2.00 equiv.), TEA (1.5 mg, 0.014 mmol, 0.10 equiv.) and iodine (43 mg, 0.168 mmol, 1.20 equiv.) at room temperature. The resulting mixture was stirred for overnight at 80° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (9 mg, 27%) as a yellow solid. ESI-MS m/z=235.03 [M+H]⁺; Calculated MW: 234.04.

### Example 11B: 4,6-dimethoxy-N—(7-methoxy-4H-chromeno[4,3-d]thiazol-2-yl)pyrimidine-5-carboxamide

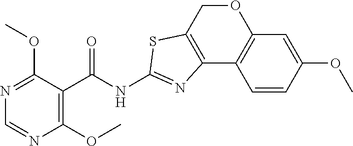

To a stirred solution of 7-methoxy-4H-chromeno[4,3-d]thiazol-2-amine (6 mg, 0.026 mmol, 1.00 equiv.), 4,6-dimethoxypyrimidine-5-carboxylic acid (12 mg, 0.064 mmol, 2.50 equiv.) and NMI (11 mg, 0.128 mmol, 5.00 equiv.) in ACN (0.2 mL) and DMF (0.2 mL) was added TCFH (18 mg, 0.064 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE:EA=4:1) to afford the title compound (6 mg, 59%) as a yellow solid. ESI-MS m/z=401.07 [M+H]⁺; Calculated MW: 400.08. ¹H NMR (400 MHz, Chloroform-d) δ 11.23 (s, 1H), 8.30 (s, 1H), 7.38 (d, J=8.4 Hz, 1H), 6.55-6.40 (m, 2H), 5.40 (s, 2H), 3.96 (s, 6H), 3.80 (s, 3H).

### Example 12: tert-butyl 2-(4,6-dimethoxypyrimidine-5-carboxamido)-7-(trifluoromethyl)spiro[chromeno[4,3-d]thiazole-4,3′-piperidine]-1′-carboxylate

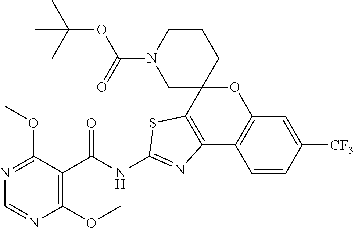 [SMILES: COc1ncnc(OC)c1C(=O)Nc1nc2c(s1)C1(CCCN(C(=O)OC(C)(C)C)C1)Oc1cc(C(F)(F)F)ccc1-2]

### Example 12A: tert-butyl 4-oxo-7-(trifluoromethyl)spiro[chromane-2,3′-piperidine]-1′-carboxylate

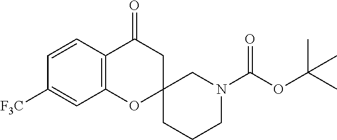 [SMILES: CC(C)(C)OC(=O)N1CCCC2(CC(=O)c3ccc(C(F)(F)F)cc3O2)C1]

To a stirred solution of 1-(2-hydroxy-4-(trifluoromethyl)phenyl)ethan-1-one (150.0 mg, 0.735 mmol, 1.00 equiv.) and tert-butyl 3-oxopiperidine-1-carboxylate (146.0 mg, 0.735 mmol, 1.00 equiv.) in MeOH (2.00 mL) was added pyrrolidine (104.0 mg, 1.47 mmol, 2.00 equiv.) in portions at 0° C. The resulting mixture was stirred for 16 h at room temperature under argon atmosphere. The reaction was diluted with water (30 mL), extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (10:1)) to afford the title compound (220 mg, 78% yield) as a light yellow oil. ¹H NMR (400 MHz, Chloroform-d) δ 7.96 (d, J=8.2 Hz, 1H), 7.25-7.20 (m, 2H), 4.08-3.87 (m, 2H), 3.04 (d, J=13.9 Hz, 2H), 2.85-2.62 (m, 2H), 2.10 (d, J=13.5 Hz, 1H), 1.95 (tt, J=11.7, 6.1 Hz, 2H), 1.70 (s, 1H), 1.31-1.13 (m, 9H).

### Example 12B: tert-butyl 2-amino-7-(trifluoromethyl)spiro[chromeno[4,3-d]thiazole-4,3′-piperidine]-1′-carboxylate

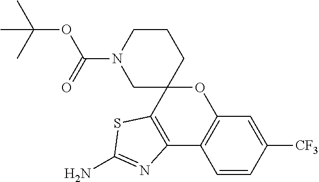 [SMILES: CC(C)(C)OC(=O)N1CCCC2(C1)Oc1cc(C(F)(F)F)ccc1-c1nc(N)sc12]

Into a 10 mL round-bottom flask were added tert-butyl 4-oxo-7-(trifluoromethyl)spiro[chromane-2,3′-piperidine]-1′-carboxylate (140 mg, 0.363 mmol, 1.00 equiv.), hexane (3 mL), molecular sieve, pyrrolidine (78 mg, 1.091 mmol, 3.00 equiv.), and TsOH·H₂O (7 mg, 0.036 mmol, 0.10 equiv.) at room temperature. The resulting mixture was stirred for 3 h at 80° C. under argon atmosphere. The resulting mixture was cooled down to room temperature, then concentrated under reduced pressure. To the above mixture was added MeOH (2 mL), S (29 mg, 0.909 mmol, 2.50 equiv.) and NH₂CN (75 mg, 0.909 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (25 mg, 15.6% yield) as a light yellow oil. ESI-MS m/z=442.17 [M+H]⁺; Calculated MW: 441.13

### Example 12C: tert-butyl 2-(4,6-dimethoxypyrimidine-5-carboxamido)-7-(trifluoromethyl)spiro[chromeno[4,3-d]thiazole-4,3′-piperidine]-1′-carboxylate

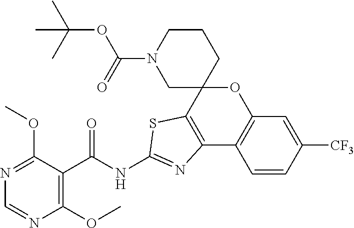 [SMILES: COc1ncnc(OC)c1C(=O)Nc1nc2c(s1)C1(CCCN(C(=O)OC(C)(C)C)C1)Oc1cc(C(F)(F)F)ccc1-2]

To a stirred solution of 4,6-dimethoxypyrimidine-5-carboxylic acid (12.00 mg, 0.062 mmol, 1.10 equiv.) in DCM (1.00 mL) was added (COCl)₂ (16.00 mg, 0.124 mmol, 2.20 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL). Tert-butyl 2-amino-7-(trifluoromethyl)spiro[chromeno[4,3-d]thiazole-4,3′-piperidine]-1′-carboxylate (25. mg, 0.057 mmol, 1.00 equiv.), TEA (23.0 mg, 0.227 mmol, 4.00 equiv.) and DMAP (7.0 mg, 0.056 mmol, 1.00 equiv.) in DCM (1.00 mL) was added in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc=3:1) to afford the title compound (7 mg, 20% yield) as an off-white solid. ESI-MS m/z=608.25 [M+H]⁺; Calculated MW: 607.17; ¹H NMR (400 MHz, Chloroform-d) δ 11.58 (d, J=55.4 Hz, 1H), 8.23 (s, 1H), 7.52 (s, 1H), 7.22-7.01 (m, 2H), 4.16-4.01 (m, 2H), 3.91 (d, J=10.4 Hz, 6H), 3.48-2.90 (m, 2H), 2.46-2.23 (m, 1H), 2.02 (d, J=11.4 Hz, 2H), 1.75-1.65 (m, 1H), 1.37-1.18 (m, 9H).

### Example 13: 4,6-dimethoxy-N—(7-(trifluoromethyl)spiro[chromeno[4,3-d]thiazole-4,1′-cyclohexan]-2-yl)pyrimidine-5-carboxamide

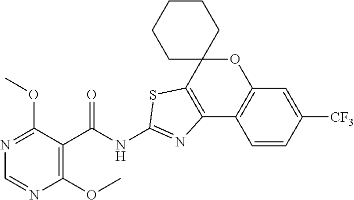 [SMILES: COc1ncnc(OC)c1C(=O)Nc1nc2c(s1)C1(CCCCC1)Oc1cc(C(F)(F)F)ccc1-2]

### Example 13A: 7-(trifluoromethyl)spiro[chromane-2,1′-cyclohexan]-4-one

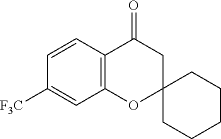 [SMILES: O=C1CC2(CCCCC2)Oc2cc(C(F)(F)F)ccc21]

To a stirred solution of 1-(2-hydroxy-4-(trifluoromethyl)phenyl)ethan-1-one (150.0 mg, 0.735 mmol, 1.00 equiv.) and cyclohexanone (72.0 mg, 0.735 mmol, 1.00 equiv.) in MeOH (2.00 mL) was added pyrrolidine (104.0 mg, 1.47 mmol, 2.00 equiv.) in portions at 0° C. The resulting mixture was stirred for 16 h at room temperature under argon atmosphere. The reaction was diluted with water (30 mL), extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (10:1)) to afford the title compound (170 mg, 81% yield) as a light yellow solid. ¹H NMR (400 MHz, Chloroform-d) δ 7.93 (dp, J=8.2, 0.8 Hz, 1H), 7.24 (d, J=0.7 Hz, 1H), 7.19 (ddt, J=8.1, 1.6, 0.7 Hz, 1H), 2.73 (s, 2H), 2.04-1.93 (m, 2H), 1.77-1.60 (m, 4H), 1.55-1.45 (m, 4H).

### Example 13B: 7-(trifluoromethyl)spiro[chromeno[4,3-d]thiazole-4,1′-cyclohexan]-2-amine

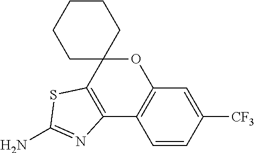 [SMILES: Nc1nc2c(s1)C1(CCCCC1)Oc1cc(C(F)(F)F)ccc1-2]

Into a 10 mL round-bottom flask were added 7-(trifluoromethyl)spiro[chromane-2,1′-cyclohexan]-4-one (170 mg, 0.599 mmol, 1.00 equiv.), EtOH (2 mL), thiourea (136 mg, 1.796 mmol, 3.00 equiv.), and I₂ (304 mg, 1.197 mmol, 2.00 equiv.) at room temperature. The resulting mixture was stirred for 16 h at 80° C. under argon atmosphere. The resulting mixture was cooled down to room temperature, adjusted pH to 9 with NaOH (1M), extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (10 mg, 5% yield) as a light yellow solid. ESI-MS m/z=341.07 [M+H]⁺; Calculated MW: 340.09

### Example 13C: 4,6-dimethoxy-N—(7-(trifluoromethyl)spiro[chromeno[4,3-d]thiazole-4,1′-cyclohexan]-2-yl)pyrimidine-5-carboxamide

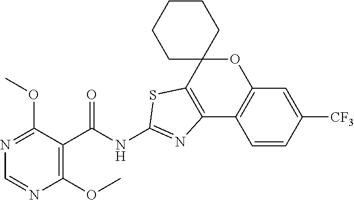 [SMILES: COc1ncnc(OC)c1C(=O)Nc1nc2c(s1)C1(CCCCC1)Oc1cc(C(F)(F)F)ccc1-2]

To a stirred solution of 4,6-dimethoxypyrimidine-5-carboxylic acid (6.0 mg, 0.033 mmol, 1.10 equiv.) in DCM (1.00 mL) was added (COCl)₂ (9.00 mg, 0.072 mmol, 2.20 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL). 7-(trifluoromethyl)spiro[chromeno[4,3-d]thiazole-4,1′-cyclohexan]-2-amine (10.00 mg, 0.030 mmol, 1.00 equiv.), TEA (12.00 mg, 0.118 mmol, 4.00 equiv.) and DMAP (4.00 mg, 0.030 mmol, 1.00 equiv.) in DCM (1.00 mL) was added in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc (4:1)) to afford the title compound (1.5 mg, 10% yield) as an off-white solid. ESI-MS m/z=507.16 [M+H]⁺; Calculated MW: 506.12; ¹H NMR (400 MHz, Chloroform-d) δ 10.75 (s, 1H), 8.38 (s, 1H), 7.62 (d, J=7.8 Hz, 1H), 7.19 (d, J=1.7 Hz, 1H), 7.18-7.14 (m, 1H), 4.03 (s, 6H), 2.28-2.18 (m, 2H), 1.92-1.69 (m, 8H).

### Example 14:2-(4,6-dimethoxypyrimidine-5-carboxamido)-4H-chromeno[4,3-d]thiazol-7-yl pivalate

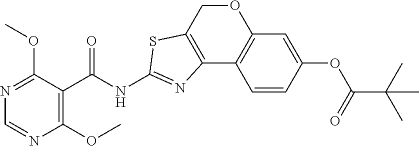 [SMILES: COc1ncnc(OC)c1C(=O)Nc1nc2c(s1)COc1cc(OC(=O)C(C)(C)C)ccc1-2]

### Example 14A: 7-hydroxychroman-4-one

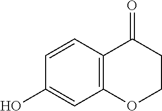 [SMILES: O=C1CCOc2cc(O)ccc21]

To a flask was added resorcinol (1 g, 0.009 mol, 1 equiv.),3-chloropropanoic acid (0.996 g, 0.009 mol, 1.011 equiv.) and trifluoromethane sulfonic acid (4.99 g, 0.033 mol, 3.66 equiv.). After stirring at 80° C. for 1 hour, the mixture was cooled to rt, quenched with water and extracted with DCM. The organic phase was wash with brine, dry with anhydrous Na₂SO₄, and concentrated to obtain a brown solid (1.21 g), which was added to 2M NaOH solution (3.86 g, 0.096 mol, 16 equiv.) at 0° C. After stirring at room temperature for 2 hours, the mixture was cooled to 0° C. and the pH was adjusted to 2 with 2M HCl. The mixture was extracted with EtOAc and the organic phase was wash with brine, dried over anhydrous sodium sulfate, and purified by silica gel column chromatography to afford the title compound (927 mg, 93%). ESI-MS m/z=164.99 [M+H]⁺; Calculated MW: 164.16.

### Example 14B: 4-oxochroman-7-yl pivalate

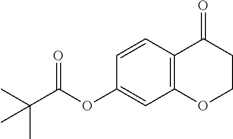 [SMILES: CC(C)(C)C(=O)Oc1ccc2c(c1)OCCC2=O]

To a flask was added 7-hydroxychroman-4-one (90 mg, 0.549 mmol, 1 equiv.), triethylamine (61 mg, 0.604 mmol, 1.1 equiv.), and DCM (1.00 mL). After cooled 0° C., pivaloyl chloride (73 mg, 0.604 mmol, 1.1 equiv.) was added dropwise. After stirring at 0° C. for 1 hour, the reaction was quenched with sat. sodium bicarbonate and extracted with. The organic phase was washed with brine, dried over anhydrous sodium sulfate, concentrated and purified by silica gel column chromatography (PE:EA=3:1) to afford the title compound (82 mg, 99%) as a brown solid. ESI-MS m/z=249.06 [M+H]⁺; Calculated MW: 248.28.

### Example 14C: 2-amino-4H-chromeno[4,3-d]thiazol-7-yl pivalate

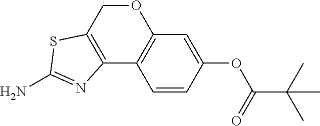 [SMILES: CC(C)(C)C(=O)Oc1ccc2c(c1)OCc1sc(N)nc1-2]

4-oxochroman-7-yl pivalate (40 mg, 0.161 mmol, 2 equiv.) was dissolved in ethanol (3 mL) at room temperature and thiourea (36.7 mg, 0.483 mmol, 3 equiv.), I₂ (90.0 mg, 0.354 mmol, 2.2 equiv.), triethylamine (1.6 mg, 0.016 mmol, 0.1 equiv.) were added. The resulting mixture was stirred overnight at 85° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=4:1) to afford the title compound (13 mg, 85%) as a brown solid. ESI-MS m/z=305.10 [M+H]⁺; Calculated MW: 304.36.

### Example 14D: 2-(4,6-dimethoxypyrimidine-5-carboxamido)-4H-chromeno[4,3-d]thiazol-7-yl pivalate

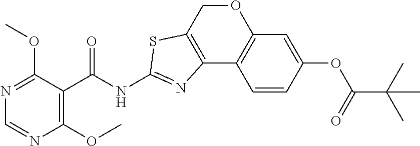 [SMILES: COc1ncnc(OC)c1C(=O)Nc1nc2c(s1)COc1cc(OC(=O)C(C)(C)C)ccc1-2]

To a stirred solution of 2-amino-4H-chromeno[4,3-d]thiazol-7-yl pivalate (13 mg, 0.043 mmol, 1.00 equiv.), 4,6-dimethoxypyrimidine-5-carboxylic acid (19.6 mg, 0.107 mmol, 2.50 equiv.) and NMI (17.5 mg, 0.213 mmol, 5.00 equiv.) in ACN (0.2 mL) and DMF (0.2 mL) was added TCFH (30.1 mg, 0.107 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE:EA=3:1) to afford the title compound (7.5 mg, 97%) as a yellow solid. ESI-MS m/z=471.12 [M+H]⁺; Calculated MW: 470.50. ¹H NMR (400 MHz, Chloroform-d) δ 11.07 (s, 1H), 8.35 (s, 1H), 7.48 (d, J=8.2 Hz, 1H), 6.66 (d, J=2.2 Hz, 1H), 5.44 (s, 2H), 3.99 (s, 6H), 1.34 (s, 8H).

### Example 15: 4,6-dimethoxy-N—(6-(trifluoromethyl)-4H-chromeno[4,3-d]thiazol-2-yl)pyrimidine-5-carboxamide

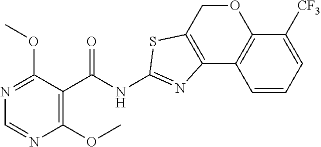 [SMILES: COc1ncnc(OC)c1C(=O)Nc1nc2c(s1)COc1c-2cccc1C(F)(F)F]

### Example 15A: 6-(trifluoromethyl)-4H-chromeno[4,3-d]thiazol-2-amine

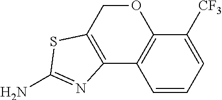 [SMILES: Nc1nc2c(s1)COc1c-2cccc1C(F)(F)F]

To a stirred solution of 8-(trifluoromethyl) chroman-4-one (50 mg, 0.231 mmol, 1.00 equiv.) in EtOH (1 mL) was added thiourea (35 mg, 0.463 mmol, 2.00 equiv.), TEA (2.3 mg, 0.023 mmol, 0.10 equiv.) and iodine (71 mg, 0.278 mmol, 1.20 equiv.) at room temperature. The resulting mixture was stirred for overnight at 80° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (13 mg, 21%) as a yellow solid. ESI-MS m/z=273.00 [M+H]⁺; Calculated MW: 272.02.

### Example 15B: 4,6-dimethoxy-N—(6-(trifluoromethyl)-4H-chromeno[4,3-d]thiazol-2-yl)pyrimidine-5-carboxamide

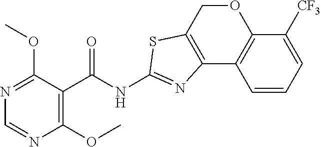 [SMILES: COc1ncnc(OC)c1C(=O)Nc1nc2c(s1)COc1c-2cccc1C(F)(F)F]

A stirred solution 6-(trifluoromethyl)-4H-chromeno[4,3-d]thiazol-2-amine (4 mg, 0.015 mmol, 1.00 equiv.), 4,6-dimethoxypyrimidine-5-carboxylic acid (7 mg, 0.037 mmol, 2.50 equiv.) and NMI (6 mg, 0.074 mmol, 5.00 equiv.) in ACN (0.2 mL) and DMF (0.2 mL) was added TCFH (10 mg, 0.037 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE:EA=4:1) to afford the title compound (3.4 mg, 53%) as a white solid. ESI-MS m/z=439.09 [M+H]⁺; Calculated MW: 438.06. ¹H NMR (400 MHz, Chloroform-d) δ 8.42 (s, 1H), 7.79-7.73 (m, 1H), 7.47-7.41 (m, 1H), 7.02 (td, J=7.8, 0.8 Hz, 1H), 5.55 (s, 2H), 4.07 (s, 6H).

### Example 16: 4,6-dimethoxy-N—(8-methyl-4H-chromeno[4,3-d]thiazol-2-yl)pyrimidine-5-carboxamide

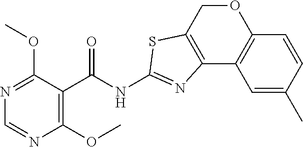

### Example 16A: 8-methyl-4H-chromeno[4,3-d]thiazol-2-amine

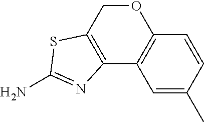

To a stirred solution of 6-methylchroman-4-one (50 mg, 0.309 mmol, 1.00 equiv.) in EtOH (1 mL) was added thiourea (47 mg, 0.617 mmol, 2.00 equiv.), TEA (3 mg, 0.031 mmol, 0.10 equiv.) and iodine (94 mg, 0.370 mmol, 1.20 equiv.) at room temperature. The resulting mixture was stirred for overnight at 80° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=4:1) to afford the title compound (33 mg, 49%) as a yellow solid. ESI-MS m/z=219.01 [M+H]⁺; Calculated MW: 218.05.

### Example 16B: 4,6-dimethoxy-N—(8-methyl-4H-chromeno[4,3-d]thiazol-2-yl)pyrimidine-5-carboxamide

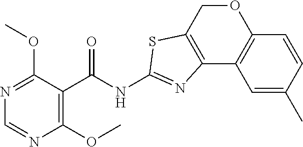

To a suspension of 4,6-dimethoxypyrimidine-5-carboxylic acid (15 mg, 0.083 mmol, 1.20 equiv.) in CH₂Cl₂ (0.5 mL) and catalytic amount of DMF, oxalyl chloride (18 mg, 0.138 mmol, 2.0 equiv.) was added dropwise at 0° C., and the mixture was stirred for 3 h at room temperature. Then the reaction mixture was concentrated in vacuo and dissolved in CH₂Cl₂ (0.5 mL). 8-methyl-4H-chromeno[4,3-d]thiazol-2-amine (15 mg, 0.069 mmol, 1.0 equiv.), TEA (28 mg, 0.275 mmol, 4.0 equiv.) and DMAP (0.84 mg, 0.0049 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=3:1) to afford the title compound (6.7 mg, 25%) as a yellow solid. ESI-MS m/z=385.11 [M+H]⁺; Calculated MW: 384.08. ¹H NMR (400 MHz, Chloroform-d) δ 11.11 (s, 1H), 8.31 (s, 1H), 7.31 (d, J=2.2 Hz, 1H), 6.96 (ddd, J=8.2, 2.2, 0.8 Hz, 1H), 6.82 (d, J=8.2 Hz, 1H), 5.38 (s, 2H), 3.96 (s, 6H), 2.27 (s, 3H).

### Example 17: 4,6-dimethoxy-N—(8-methoxy-4H-chromeno[4,3-d]thiazol-2-yl)pyrimidine-5-carboxamide

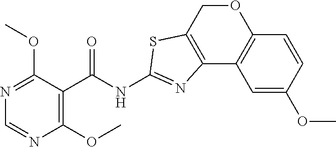

### Example 17A: 8-methoxy-4H-chromeno[4,3-d]thiazol-2-amine

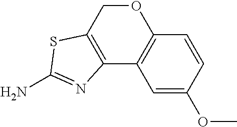 [SMILES: COc1ccc2c(c1)-c1nc(N)sc1CO2]

6-methoxychroman-4-one (50 mg, 0.227 mmol, 2 equiv.) was dissolved in ethanol (3 mL) at room temperature and thiourea (34.5 mg, 0.454 mmol, 2 equiv.), I₂ (69.2 mg, 0.272 mmol, 1.2 equiv.) and triethylamine (2.3 mg, 0.023 mmol, 0.1 equiv.) were added. The resulting mixture was stirred overnight at 85° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=5:1) to afford the title compound (25 mg, 57%) as a brown solid. ESI-MS m/z=234.99 [M+H]⁺; Calculated MW: 234.23.

### Example 17B: 4,6-dimethoxy-N—(8-methoxy-4H-chromeno[4,3-d]thiazol-2-yl)pyrimidine-5-carboxamide

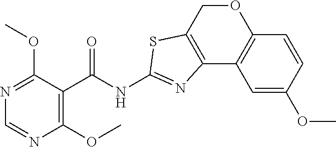

To a stirred solution of 8-methoxy-4H-chromeno[4,3-d]thiazol-2-amine (25 mg, 0.106 mmol, 1.00 equiv.), 4,6-dimethoxypyrimidine-5-carboxylic acid (49.1 mg, 0.267 mmol, 2.50 equiv.) and NMI (43.8 mg, 0.534 mmol, 5.00 equiv.) in ACN (0.2 mL) and DMF (0.2 mL) was added TCFH (74.9 mg, 0.267 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE: CH₂C₁₂: EA=3:5:1) to afford the title compound (36.7 mg, 90.3%) as a yellow solid. ESI-MS m/z=401.10 [M+H]⁺; Calculated MW: 400.40. ¹H NMR (400 MHz, Chloroform-d) δ 8.37 (s, 1H), 7.52 (dd, J=8.5, 0.8 Hz, 2H), 7.34 (t, J=2.2 Hz, 2H), 7.13-7.08 (m, 3H), 6.87 (d, J=8.8 Hz, 1H), 6.73 (dd, J=8.8, 3.0 Hz, 1H), 5.36 (s, 2H), 4.02 (s, 6H), 3.79 (s, 3H).

### Example 18: N—(4′-(tert-butyl)-7-(trifluoromethyl)spiro[chromeno[4,3-d]thiazole-4,1′-cyclohexan]-2-yl)-4,6-dimethoxypyrimidine-5-carboxamide

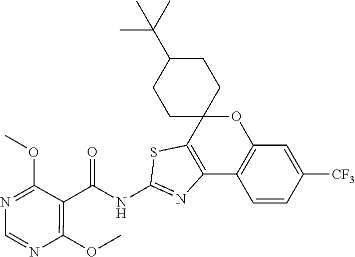 [SMILES: COc1ncnc(OC)c1C(=O)Nc1nc2c(s1)C1(CCC(C(C)(C)C)CC1)Oc1cc(C(F)(F)F)ccc1-2]

### Example 18A: 4′-(tert-butyl)-7-(trifluoromethyl)spiro[chromane-2,1′-cyclohexan]-4-one

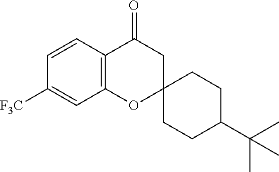 [SMILES: CC(C)(C)C1CCC2(CC1)CC(=O)c1ccc(C(F)(F)F)cc1O2]

To a stirred solution of 1-(2-hydroxy-4-(trifluoromethyl)phenyl)ethan-1-one (300.00 mg, 1.47 mmol, 1.00 equiv.) and 4-(tert-butyl)cyclohexan-1-one (227.0 mg, 1.47 mmol, 1.00 equiv.) in MeOH (3.00 mL) was added pyrrolidine (208.0 mg, 2.94 mmol, 2.00 equiv.) in portions at 0° C. The resulting mixture was stirred for 16 h at room temperature under argon atmosphere. The reaction was diluted with water (30 mL), extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (10:1)) to afford the title compound (270 mg, 54% yield) as a light yellow solid. ¹H NMR (400 MHz, Chloroform-d) δ 7.96-7.91 (m, 1H), 7.24 (dd, J=1.6, 0.8 Hz, 1H), 7.19 (ddd, J=8.2, 1.7, 0.7 Hz, 1H), 2.68 (s, 2H), 2.19-2.11 (m, 2H), 1.62 (p, J=4.2 Hz, 2H), 1.50-1.31 (m, 4H), 1.04 (tt, J=12.0, 3.4 Hz, 1H), 0.88 (s, 9H).

### Example 18B: 4′-(tert-butyl)-7-(trifluoromethyl)spiro[chromeno[4,3-d]thiazole-4,1′-cyclohexan]-2-amine

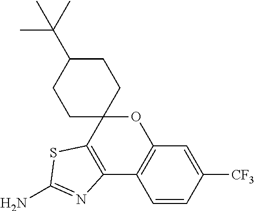 [SMILES: CC(C)(C)C1CCC2(CC1)Oc1cc(C(F)(F)F)ccc1-c1nc(N)sc12]

Into a 10 mL round-bottom flask were added 4′-(tert-butyl)-7-(trifluoromethyl)spiro[chromane-2,1′-cyclohexan]-4-one (270 mg, 0.794 mmol, 1.00 equiv.), EtOH (2 mL), thiourea (181 mg, 2.38 mmol, 3.00 equiv.), and I₂ (403 mg, 1.588 mmol, 2.00 equiv.) at room temperature. The resulting mixture was stirred for 16 h at 80° C. under argon atmosphere. The resulting mixture was cooled down to room temperature, adjusted pH to 9 with NaOH (1M), extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (19 mg, 6% yield) as a light yellow solid. ESI-MS m/z=397.14 [M+H]⁺; Calculated MW: 396.15

### Example 18C: N—(4′-(tert-butyl)-7-(trifluoromethyl)spiro[chromeno[4,3-d]thiazole-4,1′-cyclohexan]-2-yl)-4,6-dimethoxypyrimidine-5-carboxamide

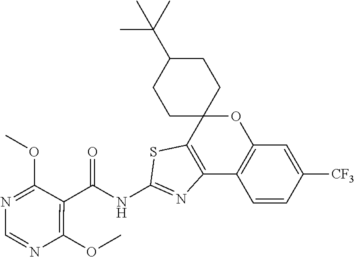 [SMILES: COc1ncnc(OC)c1C(=O)Nc1nc2c(s1)C1(CCC(C(C)(C)C)CC1)Oc1cc(C(F)(F)F)ccc1-2]

To a stirred solution of 4,6-dimethoxypyrimidine-5-carboxylic acid (10.0 mg, 0.053 mmol, 1.10 equiv.) in DCM (1.00 mL) was added (COCl)₂ (31.00 mg, 0.240 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL). 4′-(tert-butyl)-7-(trifluoromethyl)spiro[chromeno[4,3-d]thiazole-4,1′-cyclohexan]-2-amine (19.00 mg, 0.048 mmol, 1.00 equiv.), TEA (49.00 mg, 0.480 mmol, 10.00 equiv.) and DMAP (6.00 mg, 0.048 mmol, 1.00 equiv.) in DCM (1.00 mL) was added in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc/DCM (7:1:4)) to afford the title compound (3.5 mg, 13% yield) as a light yellow solid. ESI-MS m/z=563.23 [M+H]⁺; Calculated MW: 562.19; ¹H NMR (400 MHz, Chloroform-d) δ 11.24 (d, J=19.9 Hz, 1H), 8.31-8.26 (m, 1H), 7.54 (dd, J=7.9, 3.4 Hz, 1H), 7.16 (d, J=1.7 Hz, 1H), 7.11 (dt, J=7.9, 2.1 Hz, 1H), 3.95 (d, J=3.5 Hz, 6H), 2.36 (dd, J=7.7, 5.0 Hz, 2H), 1.72-1.61 (m, 6H), 1.18-1.07 (m, 1H), 0.93 (s, 9H).

### Example 19: 4,6-dimethoxy-N—(7-(trifluoromethyl)-3a,9b-dihydrospiro[chromeno[4,3-d]thiazole-4,1′-cyclobutan]-2-yl)pyrimidine-5-carboxamide

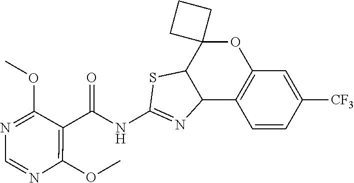 [SMILES: COc1ncnc(OC)c1C(=O)NC1=NC2c3ccc(C(F)(F)F)cc3OC3(CCC3)C2S1]

### Example 19A: 7-(trifluoromethyl)spiro[chromane-2,1′-cyclobutan]-4-one

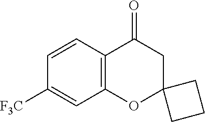 [SMILES: O=C1CC2(CCC2)Oc2cc(C(F)(F)F)ccc21]

To a stirred solution of 1-(2-hydroxy-4-(trifluoromethyl)phenyl)ethan-1-one (150.0 mg, 0.735 mmol, 1.00 equiv.) and cyclobutanone (52.0 mg, 0.735 mmol, 1.00 equiv.) in MeOH (2.00 mL) was added pyrrolidine (104.0 mg, 1.47 mmol, 2.00 equiv.) in portions at 0° C. The resulting mixture was stirred for 16 h at room temperature under argon atmosphere. The reaction was diluted with water (30 mL), extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (10:1)) to afford the title compound (76 mg, 40% yield) as a light yellow solid. ¹H NMR (400 MHz, Chloroform-d) δ 7.94 (dt, J=7.9, 0.9 Hz, 1H), 7.26-7.25 (m, 1H), 7.21 (dd, J=7.9, 1.7 Hz, 1H), 2.93 (s, 2H), 2.34 (qd, J=9.8, 2.7 Hz, 2H), 2.18 (ddtd, J=12.7, 6.2, 3.4, 1.8 Hz, 2H), 2.00-1.89 (m, 1H), 1.79-1.67 (m, 1H).

### Example 19B: 7-(trifluoromethyl)spiro[chromeno[4,3-d]thiazole-4,1′-cyclobutan]-2-amine

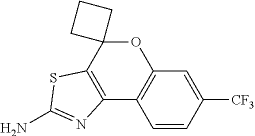 [SMILES: Nc1nc2c(s1)C1(CCC1)Oc1cc(C(F)(F)F)ccc1-2]

Into a 10 mL round-bottom flask were added 7-(trifluoromethyl)spiro[chromane-2,1′-cyclobutan]-4-one (76 mg, 0.297 mmol, 1.00 equiv.), EtOH (2 mL), thiourea (68 mg, 0.891 mmol, 3.00 equiv.), and I₂ (151 mg, 0.594 mmol, 2.00 equiv.) at room temperature. The resulting mixture was stirred for 16 h at 80° C. under argon atmosphere. The resulting mixture was cooled down to room temperature, adjusted PH to 9 with NaOH (1M), extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (5 mg, 5% yield) as a light yellow solid. ESI-MS m/z=313.06 [M+H]⁺; Calculated MW: 312.05

### Example 19C

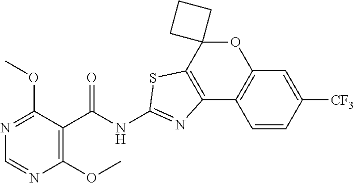 [SMILES: COc1ncnc(OC)c1C(=O)Nc1nc2c(s1)C1(CCC1)Oc1cc(C(F)(F)F)ccc1-2]

To a stirred solution of 4,6-dimethoxypyrimidine-5-carboxylic acid (3.50 mg, 0.019 mmol, 1.20 equiv.) in DCM (1.00 mL) was added (COCl)₂ (10.00 mg, 0.080 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL). 7-(trifluoromethyl)spiro[chromeno[4,3-d]thiazole-4,1′-cyclobutan]-2-amine (5.00 mg, 0.016 mmol, 1.00 equiv.), TEA (16.00 mg, 0.160 mmol, 10.00 equiv.) and DMAP (2.00 mg, 0.016 mmol, 1.00 equiv.) in DCM (1.00 mL) was added in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc/DCM (7:1:4)) to afford the title compound (1.0 mg, 13% yield) as a light yellow solid. ESI-MS m/z=479.13 [M+H]⁺; Calculated MW: 478.09; 1H NMR (400 MHz, Chloroform-d) δ 10.25 (s, 1H), 8.49 (s, 1H), 7.70 (d, J=7.8 Hz, 1H), 7.24-7.16 (m, 2H), 4.13 (d, J=2.5 Hz, 6H), 2.74 (q, J=10.5, 10.1 Hz, 2H), 2.51 (s, 2H), 2.25-2.17 (m, 2H).

### Example 20: 4,6-dimethoxy-N—(7′-(trifluoromethyl)spiro[adamantane-2,4′-chromeno[4,3-d]thiazol]-2′-yl)pyrimidine-5-carboxamide

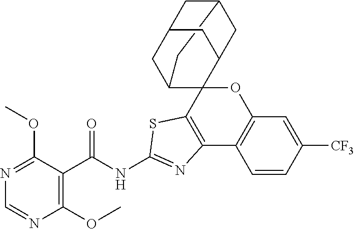 [SMILES: COc1ncnc(OC)c1C(=O)Nc1nc2c(s1)C1(Oc3cc(C(F)(F)F)ccc3-2)C2CC3CC(C2)CC1C3]

### Example 20A: 7′-(trifluoromethyl)spiro[adamantane-2,2′-chroman]-4′-one

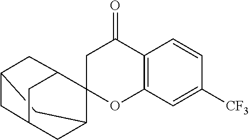 [SMILES: O=C1CC2(Oc3cc(C(F)(F)F)ccc31)C1CC3CC(C1)CC2C3]

At 0° C., 1-(2-hydroxy-4-(trifluoromethyl)phenyl)ethan-1-one (300 mg, 1.471 mmol) was dissolved in methanol (3 mL). Adamantan-2-one (221.0 mg, 1.471 mmol) and tetrahydropyrrole (208.8 mg, 2.941 mmol) were added and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (210 mg, 42.5%) as a yellow solid.

### Example 20B: 7′-(trifluoromethyl)spiro[adamantane-2,4′-chromeno[4,3-d]thiazol]-2′-amine

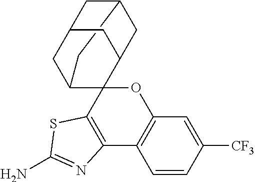 [SMILES: Nc1nc2c(s1)C1(Oc3cc(C(F)(F)F)ccc3-2)C2CC3CC(C2)CC1C3]

Into a 10 mL round-bottom flask were added 7′-(trifluoromethyl)spiro[adamantane-2,2′-chroman]-4′-one (300 mg, 0.893 mmol, 1.00 equiv.), hexane (5 mL), molecular sieve, pyrrolidine (317 mg, 4.46 mmol, 5.00 equiv.), and TsOH·H₂O (17 mg, 0.089 mmol, 0.10 equiv.) at room temperature. The resulting mixture was stirred for 3 h at 80° C. under argon, cooled down to room temperature, then concentrated under reduced pressure. To the above mixture was added MeOH (2 mL), S (72 mg, 2.23 mmol, 2.50 equiv.) and NH₂CN (184 mg, 2.23 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc=3:1) to afford the title compound (10 mg, 2.8% yield) as a light yellow oil. ESI-MS m/z=393.11 [M+H]⁺; Calculated MW: 392.12

### Example 20C: 4,6-dimethoxy-N—(7′-(trifluoromethyl)spiro[adamantane-2,4′-chromeno[4,3-d]thiazol]-2′-yl)pyrimidine-5-carboxamide

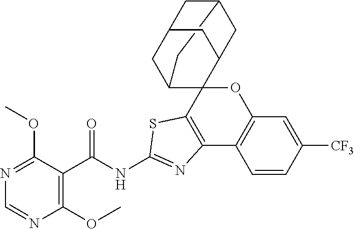 [SMILES: COc1ncnc(OC)c1C(=O)Nc1nc2c(s1)C1(Oc3cc(C(F)(F)F)ccc3-2)C2CC3CC(C2)CC1C3]

To a stirred solution of 4,6-dimethoxypyrimidine-5-carboxylic acid (12.0 mg, 0.062 mmol, 1.10 equiv.) in DCM (1.00 mL) was added (COCl)₂ (37.0 mg, 0.293 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL). 7′-(trifluoromethyl)spiro[adamantane-2,4′-chromeno[4,3-d]thiazol]-2′-amine (22.0 mg, 0.058 mmol, 1.00 equiv.), TEA (59.0 mg, 0.585 mmol, 10.00 equiv.) and DMAP (7.0 mg, 0.058 mmol, 1.00 equiv.) in DCM (1.00 mL) was added in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (DCM/PE/EtOAc (7:4:1)) to afford the title compound (1.9 mg, 13% yield) as an off-white solid. ESI-MS m/z=559.20 [M+H]⁺; Calculated MW: 558.19; 1H NMR (400 MHz, Chloroform-d) δ 10.55 (s, 1H), 8.44 (s, 1H), 7.72 (d, J=7.9 Hz, 1H), 7.56-7.46 (m, 1H), 7.11 (dd, J=8.6, 2.5 Hz, 1H), 4.08 (s, 6H), 2.46 (d, J=12.7 Hz, 2H), 2.33 (t, J=7.5 Hz, 1H), 2.28 (s, 1H), 2.23-2.15 (m, 4H), 1.98 (dd, J=15.2, 8.8 Hz, 3H), 1.86 (d, J=13.8 Hz, 1H), 1.77 (s, 2H).

### Example 21: 4,6-dimethoxy-N—(7-(trifluoromethyl)-4′,5′-dihydro-2′H-spiro[chromeno[4,3-d]thiazole-4,3′-furan]-2-yl)pyrimidine-5-carboxamide

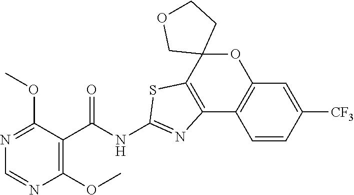 [SMILES: COc1ncnc(OC)c1C(=O)Nc1nc2c(s1)C1(CCOC1)Oc1cc(C(F)(F)F)ccc1-2]

### Example 21A: 7-(trifluoromethyl)-4′,5′-dihydro-2′H-spiro[chromane-2,3′-furan]-4-one

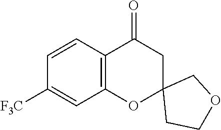 [SMILES: O=C1CC2(CCOC2)Oc2cc(C(F)(F)F)ccc21]

At 0° C., 1-(2-hydroxy-4-(trifluoromethyl)phenyl)ethan-1-one (300 mg, 1.471 mmol) was dissolved in methanol (3 mL), dihydrofuran-3 (2H)-one (190 mg, 2.2 mmol) and tetrahydropyrrole (208.8 mg, 2.941 mmol) were added, the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE:EA=15:1) to afford the title compound (182 mg, 45.5%) as a yellow oil. ¹H NMR (400 MHz, Chloroform-d) δ 7.98 (dd, J=8.2, 1.1 Hz, 1H), 7.25 (d, J=0.7 Hz, 1H), 4.16-4.10 (m, 1H), 4.01 (dd, J=8.6, 3.9 Hz, OH), 3.96-3.86 (m, OH), 3.73 (d, J=10.1 Hz, OH), 2.98 (dd, J=16.5, 0.7 Hz, 1H), 2.69 (t, J=7.8 Hz, OH), 2.01 (d, J=13.4 Hz, OH).

### Example 21B: 7-(trifluoromethyl)-4′,5′-dihydro-2′H-spiro[chromeno[4,3-d]thiazole-4,3′-furan]-2-amine

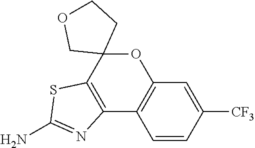 [SMILES: Nc1nc2c(s1)C1(CCOC1)Oc1cc(C(F)(F)F)ccc1-2]

7-(trifluoromethyl)-4′,5′-dihydro-2′H-spiro[chromane-2,3′-furan]-4-one (182 mg, 0.669 mmol) was dissolved in ethanol (3 mL) at room temperature and thiourea (101.6 mg, 1.337 mmol), I₂ (203.8 mg, 0.802 mmol), triethylamine (6.7 mg, 0.067 mmol) were added. The resulting mixture was stirred overnight at 85° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=6:1) to afford the title compound (10 mg, 95%) as a brown solid. ESI-MS m/z=329.10 [M+H]⁺; Calculated MW: 328.31.

### Example 21C: 4,6-dimethoxy-N—(7-(trifluoromethyl)-4′,5′-dihydro-2′H-spiro[chromeno[4,3-d]thiazole-4,3′-furan]-2-yl)pyrimidine-5-carboxamide

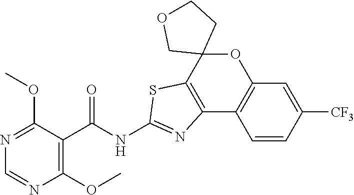 [SMILES: COc1ncnc(OC)c1C(=O)Nc1nc2c(s1)C1(CCOC1)Oc1cc(C(F)(F)F)ccc1-2]

To a stirred solution of 4,6-dimethoxypyrimidine-5-carboxylic acid (13.0 mg, 0.067 mmol, 1.10 equiv.) in DCM (1.00 mL) was added (COCl)₂ (39.00 mg, 0.305 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL), then added to a stirred solution of 7-(trifluoromethyl)-4′,5′-dihydro-2′H-spiro[chromeno[4,3-d]thiazole-4,3′-furan]-2-amine (20.0 mg, 0.061 mmol, 1.00 equiv.), TEA (62.0 mg, 0.610 mmol, 10 equiv.) and DMAP (7.5 mg, 0.061 mmol, 1.00 equiv.) in DCM (1.00 mL) in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc/DCM (7:1:4)) to afford the title compound (5.0 mg, 16% yield) as an off-white solid. ESI-MS m/z=495.15 [M+H]⁺; Calculated MW: 494.09; ¹H NMR (400 MHz, Chloroform-d) δ 11.00 (s, 1H), 8.35 (s, 1H), 7.61 (dt, J=7.8, 0.8 Hz, 1H), 7.21-7.15 (m, 2H), 4.31-4.21 (m, 2H), 4.13 (td, J=8.7, 3.7 Hz, 1H), 4.01 (s, 6H), 3.88 (d, J=10.2 Hz, 1H), 2.68 (dddd, J=13.5, 6.8, 3.7, 1.2 Hz, 1H), 2.36-2.25 (m, 1H).

### Example 22: 4,6-dimethoxy-N—(7-(trifluoromethyl)-4,5-dihydronaphtho[1,2-d]thiazol-2-yl)pyrimidine-5-carboxamide

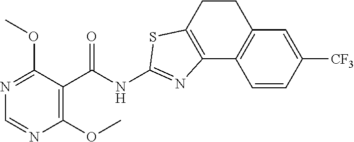 [SMILES: COc1ncnc(OC)c1C(=O)Nc1nc2c(s1)CCc1cc(C(F)(F)F)ccc1-2]

### Example 22A: 7-(trifluoromethyl)-4,5-dihydronaphtho[1,2-d]thiazol-2-amine

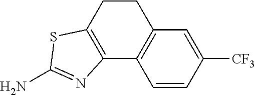 [SMILES: Nc1nc2c(s1)CCc1cc(C(F)(F)F)ccc1-2]

To a stirred solution of 6-(trifluoromethyl)-3,4-dihydronaphthalen-1 (2H)-one (50 mg, 0.234 mmol, 1.00 equiv.) in EtOH (1 mL) was added thiourea (36 mg, 0.467 mmol, 2.00 equiv.), TEA (2 mg, 0.023 mmol, 0.10 equiv.) and iodine (71 mg, 0.280 mmol, 1.20 equiv.) at room temperature. The resulting mixture was stirred for overnight at 80° C. Then it was cooled to room temperature and the resulting mixture was extracted with EA (2×15 mL). The combined organic layers were washed with 1N NaOH (1×5 mL) and brine (1×10 mL), dried over anhydrous Na₂SO₄. After filtration, the filtrate was concentrated under reduced pressure. The crude product was purified by prep-TLC (PE:EA=5:1) to afford the title compound (33 mg, 55%) as a brown solid. ESI-MS m/z=271.01 [M+H]⁺; Calculated MW: 270.04.

### Example 22B: 4,6-dimethoxy-N—(7-(trifluoromethyl)-4,5-dihydronaphtho[1,2-d]thiazol-2-yl)pyrimidine-5-carboxamide

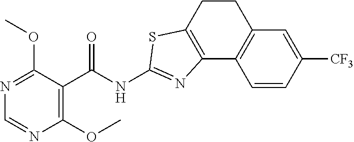 [SMILES: COc1ncnc(OC)c1C(=O)Nc1nc2c(s1)CCc1cc(C(F)(F)F)ccc1-2]

To a suspension of 4,6-dimethoxypyrimidine-5-carboxylic acid (12 mg, 0.067 mmol, 1.20 equiv.) in CH₂Cl₂ (0.5 mL) and catalytic amount of DMF, oxalyl chloride (14 mg, 0.111 mmol, 2.0 equiv.) was added dropwise at 0° C., and the mixture was stirred for 3 h at room temperature. Then the reaction mixture was concentrated in vacuo and dissolved in CH₂Cl₂ (0.5 mL). 7-(trifluoromethyl)-4,5-dihydronaphtho[1,2-d]thiazol-2-amine (15 mg, 0.056 mmol, 1.0 equiv.), TEA (23 mg, 0.222 mmol, 4.0 equiv.) and DMAP (0.68 mg, 0.0056 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (8 mg, 33%) as a white solid. ESI-MS m/z=437.09 [M+H]⁺; Calculated MW: 436.08. ¹H NMR (400 MHz, Chloroform-d) δ 12.23 (s, 1H), 8.04 (d, J=1.0 Hz, 1H), 7.55 (d, J=8.0 Hz, 1H), 7.42 (s, 1H), 7.36 (d, J=8.0 Hz, 1H), 3.79 (d, J=1.1 Hz, 6H), 3.13-2.96 (m, 4H).

### Example 23: 4,6-dimethoxy-N—(7-(trifluoromethyl)spiro[chromeno[4,3-d]thiazole-4,1′-cyclopentan]-2-yl)pyrimidine-5-carboxamide

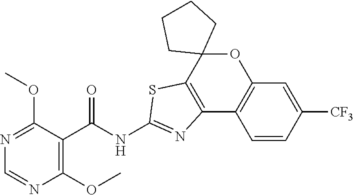 [SMILES: COc1ncnc(OC)c1C(=O)Nc1nc2c(s1)C1(CCCC1)Oc1cc(C(F)(F)F)ccc1-2]

### Example 23A: 7-(trifluoromethyl)spiro[chromane-2,1′-cyclopentan]-4-one

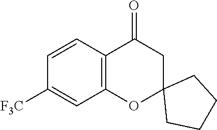 [SMILES: O=C1CC2(CCCC2)Oc2cc(C(F)(F)F)ccc21]

At 0° C., 1-(2-hydroxy-4-(trifluoromethyl)phenyl)ethan-1-one (150 mg, 0.735 mmol) was dissolved in methanol (3 mL), cyclopentanone (61.8 mg, 0.735 mmol) and tetrahydropyrrole (104.4 mg, 1.471 mmol) were added, the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE:EA=15:1) to afford the title compound (120 mg, 60.6%) as a yellow oil. ¹H NMR (400 MHz, Chloroform-d) δ 7.95 (dq, J=7.6, 1.0 Hz, 1H), 7.20 (d, J=1.8 Hz, 2H), 2.86 (s, 2H), 2.15-2.03 (m, 2H), 1.92-1.84 (m, 2H), 1.78-1.65 (m, 2H).

### Example 23B: 7-(trifluoromethyl)spiro[chromeno[4,3-d]thiazole-4,1′-cyclopentan]-2-amine

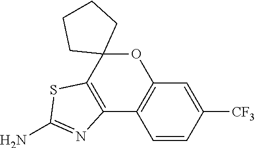 [SMILES: Nc1nc2c(s1)C1(CCCC1)Oc1cc(C(F)(F)F)ccc1-2]

7-(trifluoromethyl)spiro[chromane-2,1′-cyclopentan]-4-one (120 mg, 0.444 mmol) was dissolved in ethanol (3 mL) at room temperature and thiourea (67.5 mg, 0.888 mmol), I₂ (135.4 mg, 0.533 mmol), triethylamine (4.5 mg, 0.044 mmol) were added. The resulting mixture was stirred overnight at 85° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=6:1) to afford the title compound (10 mg, 98%) as a brown solid. ESI-MS m/z=327.07 [M+H]⁺; Calculated MW: 326.34.

### Example 23C: 4,6-dimethoxy-N—(7-(trifluoromethyl)spiro[chromeno[4,3-d]thiazole-4,1′-cyclopentan]-2-yl)pyrimidine-5-carboxamide

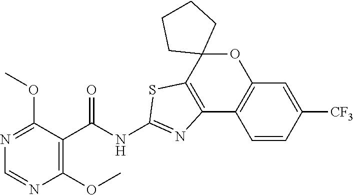 [SMILES: COc1ncnc(OC)c1C(=O)Nc1nc2c(s1)C1(CCCC1)Oc1cc(C(F)(F)F)ccc1-2]

To a stirred solution of 4,6-dimethoxypyrimidine-5-carboxylic acid (9.0 mg, 0.048 mmol, 1.20 equiv.) in DCM (1.00 mL) was added (COCl)₂ (25.0 mg, 0.199 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL), then added to a stirred solution of 7-(trifluoromethyl)spiro[chromeno[4,3-d]thiazole-4,1′-cyclopentan]-2-amine (13.0 mg, 0.040 mmol, 1.00 equiv.), TEA (40.0 mg, 0.400 mmol, 10.00 equiv.) and DMAP (5.0 mg, 0.040 mmol, 1.00 equiv.) in DCM (1.00 mL) in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc/DCM (7:1:4)) to afford the title compound (2.0 mg, 10% yield) as a light yellow solid. ESI-MS m/z=493.14 [M+H]⁺; Calculated MW: 492.11; 1H NMR (400 MHz, Chloroform-d) δ 10.99 (s, 1H), 8.34 (s, 1H), 7.59 (d, J=7.7 Hz, 1H), 7.16-7.11 (m, 2H), 3.99 (s, 6H), 2.44-2.32 (m, 3H), 2.07-1.97 (m, 2H), 1.96-1.80 (m, 3H).

### Example 24: N—(4,4-diethyl-7-(trifluoromethyl)-4H-chromeno[4,3-d]thiazol-2-yl)-4,6-dimethoxypyrimidine-5-carboxamide

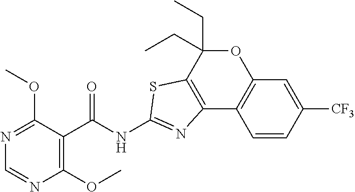 [SMILES: CCC1(CC)Oc2cc(C(F)(F)F)ccc2-c2nc(NC(=O)c3c(OC)ncnc3OC)sc21]

### Example 24A: 2,2-diethyl-7-(trifluoromethyl) chroman-4-one

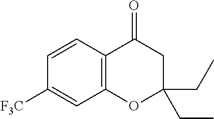 [SMILES: CCC1(CC)CC(=O)c2ccc(C(F)(F)F)cc2O1]

At 0° C., 1-(2-hydroxy-4-(trifluoromethyl)phenyl)ethan-1-one (300 mg, 1.471 mmol) was dissolved in methanol (3 mL), pentan-3-one (126.7 mg, 1.471 mmol) and tetrahydropyrrole (208.8 mg, 2.941 mmol) were added, the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (183 mg, 45.7%) as a yellow oil.

### Example 24B: 4,4-diethyl-7-(trifluoromethyl)-4H-chromeno[4,3-d]thiazol-2-amine

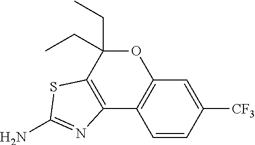 [SMILES: CCC1(CC)Oc2cc(C(F)(F)F)ccc2-c2nc(N)sc21]

Into a 10 mL round-bottom flask were added 2,2-diethyl-7-(trifluoromethyl) chroman-4-one (160 mg, 0.588 mmol, 1.00 equiv.), hexane (5 mL), molecular sieve, pyrrolidine (209 mg, 2.94 mmol, 5.00 equiv.), and TsOH·H₂O (11 mg, 0.059 mmol, 0.10 equiv.) at room temperature. The resulting mixture was stirred for 3 h at 80° C. under argon atmosphere, cooled down to room temperature, then concentrated under reduced pressure. To the above mixture was added MeOH (2 mL), S (47 mg, 1.470 mmol, 2.50 equiv.) and NH₂CN (121 mg, 1.470 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc=3:1) to afford the title compound (20 mg, 10% yield) as a yellow brown oil. ESI-MS m/z=329.08 [M+H]⁺; Calculated MW: 328.09

### Example 24C: N—(4,4-diethyl-7-(trifluoromethyl)-4H-chromeno[4,3-d]thiazol-2-yl)-4,6-dimethoxypyrimidine-5-carboxamide

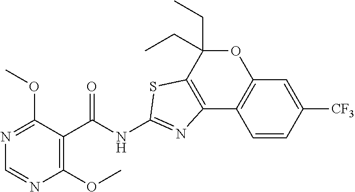 [SMILES: CCC1(CC)Oc2cc(C(F)(F)F)ccc2-c2nc(NC(=O)c3c(OC)ncnc3OC)sc21]

To a stirred solution of 4,6-dimethoxypyrimidine-5-carboxylic acid (11.0 mg, 0.060 mmol, 1.10 equiv.) in DCM (1.0 mL) was added (COCl)₂ (35.0 mg, 0.274 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (18.0 mg, 0.055 mmol, 1.00 equiv.) in THF (1.0 mL) was added NaH (7.0 mg, 0.165 mmol, 3.0 equiv., 60%) in portions at 0° C. The resulting mixture was stirred for 0.5 h at 0° C. under argon atmosphere. To the above mixture was added the acyl chloride/THF mixture dropwise at 0° C. The resulting mixture was stirred for additional 16 h at 0° C. to room temperature, quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (10 mg, 20% yield) as a light yellow solid. ESI-MS m/z=495.15 [M+H]⁺; Calculated MW: 494.12; ¹H NMR (400 MHz, Chloroform-d) δ 11.34 (s, 1H), 8.27 (s, 1H), 7.51 (d, J=7.9 Hz, 1H), 7.10 (d, J=1.7 Hz, 1H), 7.05 (dd, J=7.9, 1.7 Hz, 1H), 3.96 (s, 6H), 2.02 (dq, J=14.8, 7.4 Hz, 2H), 1.90 (dq, J=14.6, 7.4 Hz, 2H), 0.99 (t, J=7.4 Hz, 6H).

### Example 25: 4,6-dimethoxy-N—(4-methyl-4-phenyl-7-(trifluoromethyl)-4H-chromeno[4,3-d]thiazol-2-yl)pyrimidine-5-carboxamide

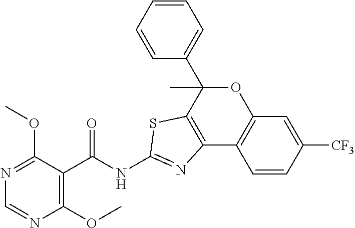 [SMILES: COc1ncnc(OC)c1C(=O)Nc1nc2c(s1)C(C)(c1ccccc1)Oc1cc(C(F)(F)F)ccc1-2]

### Example 25A: 2-methyl-2-phenyl-7-(trifluoromethyl) chroman-4-one

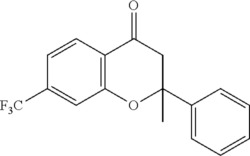 [SMILES: CC1(c2ccccc2)CC(=O)c2ccc(C(F)(F)F)cc2O1]

At 0° C., 1-(2-hydroxy-4-(trifluoromethyl)phenyl)ethan-1-one (300 mg, 1.471 mmol) was dissolved in methanol (3 mL), acetophenone (176.7 mg, 1.471 mmol) and tetrahydropyrrole (208.8 mg, 2.941 mmol) were added, the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (100 mg) as a yellow oil. ¹H NMR (400 MHz, Chloroform-d) δ 7.98-7.93 (m, 1H), 7.38 (d, J=1.0 Hz, 2H), 7.31 (s, 1H), 3.36 (d, J=16.5 Hz, 2H), 1.77 (s, 5H).

### Example 25B: 4-methyl-4-phenyl-7-(trifluoromethyl)-4H-chromeno[4,3-d]thiazol-2-amine

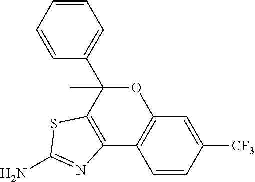 [SMILES: CC1(c2ccccc2)Oc2cc(C(F)(F)F)ccc2-c2nc(N)sc21]

Into a 10 mL round-bottom flask were added 2-methyl-2-phenyl-7-(trifluoromethyl) chroman-4-one (200 mg, 0.653 mmol, 1.00 equiv.), hexane (5 mL), molecular sieve, pyrrolidine (232 mg, 3.268 mmol, 5.00 equiv.), and TsOH·H₂O (12 mg, 0.065 mmol, 0.10 equiv.) at room temperature. The resulting mixture was stirred for 3 h at 80° C. under argon atmosphere, cooled down to room temperature, then concentrated under reduced pressure. To the above mixture was added MeOH (2 mL), S (52 mg, 1.634 mmol, 2.50 equiv.) and NH₂CN (135 mg, 1.634 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc=3:1) to afford the title compound (32 mg, 14% yield) as a yellow brown oil. ESI-MS m/z=363.08 [M+H]⁺; Calculated MW: 362.07

### Example 25C: 4,6-dimethoxy-N—(4-methyl-4-phenyl-7-(trifluoromethyl)-4H-chromeno[4,3-d]thiazol-2-yl)pyrimidine-5-carboxamide

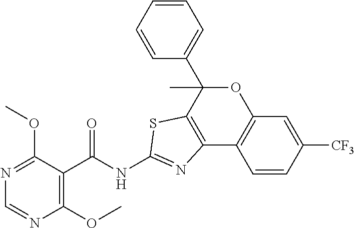 [SMILES: COc1ncnc(OC)c1C(=O)Nc1nc2c(s1)C(C)(c1ccccc1)Oc1cc(C(F)(F)F)ccc1-2]

To a stirred solution of 4,6-dimethoxypyrimidine-5-carboxylic acid (18.0 mg, 0.097 mmol, 1.10 equiv.) in DCM (1.00 mL) was added (COCl)₂ (56.0 mg, 0.442 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4-methyl-4-phenyl-7-(trifluoromethyl)-4H-chromeno[4,3-d]thiazol-2-amine (32.0 mg, 0.088 mmol, 1.00 equiv.) in THF (1.0 mL) was added NaH (11.0 mg, 0.265 mmol, 3.0 equiv., 60%) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (9.3 mg, 20% yield) as an off-white solid. ESI-MS m/z=529.14 [M+H]⁺; Calculated MW: 528.11; ¹H NMR (400 MHz, Chloroform-d) δ 11.79 (s, 1H), 8.20 (d, J=2.4 Hz, 1H), 7.53-7.43 (m, 3H), 7.39-7.25 (m, 3H), 7.22 (d, J=1.7 Hz, 1H), 7.06 (d, J=8.0 Hz, 1H), 3.88 (d, J=2.3 Hz, 6H), 2.10 (d, J=2.3 Hz, 3H).

### Example 26: 4,6-dimethoxy-N—(7-(trifluoromethyl)spiro[chromeno[4,3-d]thiazole-4,1′-cycloheptan]-2-yl)pyrimidine-5-carboxamide

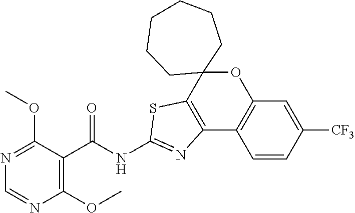 [SMILES: COc1ncnc(OC)c1C(=O)Nc1nc2c(s1)C1(CCCCCC1)Oc1cc(C(F)(F)F)ccc1-2]

### Example 26A: 7-(trifluoromethyl)spiro[chromane-2,1′-cycloheptan]-4-one

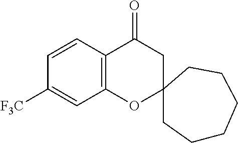 [SMILES: O=C1CC2(CCCCCC2)Oc2cc(C(F)(F)F)ccc21]

At 0° C., 1-(2-hydroxy-4-(trifluoromethyl)phenyl)ethan-1-one (150 mg, 0.735 mmol) was dissolved in methanol (3 mL), cycloheptanone (82.5 mg, 0.735 mmol) and tetrahydropyrrole (104.4 mg, 1.471 mmol) were added, the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE:EA=15:1)) to afford the title compound (137 mg, 62.5%) as a yellow oil. ¹H NMR (400 MHz, Chloroform-d) δ 7.92 (dd, J=8.1, 1.0 Hz, 1H), 7.23-7.15 (m, 1H), 2.76 (s, 1H), 1.75-1.49 (m, 4H).

### Example 26B: 7-(trifluoromethyl)spiro[chromeno[4,3-d]thiazole-4,1′-cycloheptan]-2-amine

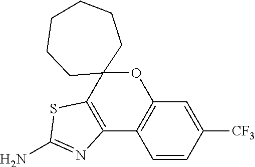 [SMILES: Nc1nc2c(s1)C1(CCCCCC1)Oc1cc(C(F)(F)F)ccc1-2]

7-(trifluoromethyl)spiro[chromane-2,1′-cycloheptan]-4-one (137 mg, 0.459 mmol) was dissolved in ethanol (3 mL) at room temperature and thiourea (69.8 mg, 0.919 mmol), I₂ (140 mg, 0.551 mmol), triethylamine (4.7 mg, 0.046 mmol) were added. The resulting mixture was stirred overnight at 85° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=6:1) to afford the title compound (4 mg, 52%) as a brown solid. ESI-MS m/z=355.12 [M+H]⁺; Calculated MW: 354.39.

### Example 26C: 4,6-dimethoxy-N—(7-(trifluoromethyl)spiro[chromeno[4,3-d]thiazole-4,1′-cycloheptan]-2-yl)pyrimidine-5-carboxamide

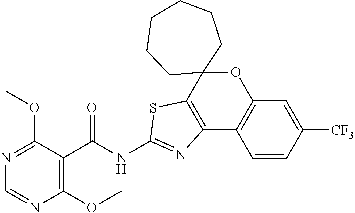 [SMILES: COc1ncnc(OC)c1C(=O)Nc1nc2c(s1)C1(CCCCCC1)Oc1cc(C(F)(F)F)ccc1-2]

To a stirred solution of 4,6-dimethoxypyrimidine-5-carboxylic acid (4.0 mg, 0.022 mmol, 1.10 equiv.) in DCM (1.0 mL) was added (COCl)₂ (14.0 mg, 0.108 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 7-(trifluoromethyl)spiro[chromeno[4,3-d]thiazole-4,1′-cycloheptan]-2-amine (7.0 mg, 0.020 mmol, 1.00 equiv.) in THF (1.00 mL) was added NaH (2.5 mg, 0.059 mmol, 3.00 equiv., 60%) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (3 mg, 29% yield) as an off-white solid. ESI-MS m/z=521.16 [M+H]⁺; Calculated MW: 520.14; ¹H NMR (400 MHz, Chloroform-d) δ 11.17 (s, 1H), 8.30 (s, 1H), 7.57-7.52 (m, 1H), 7.15 (d, J=1.7 Hz, 1H), 7.13-7.09 (m, 1H), 3.96 (s, 6H), 2.38-2.27 (m, 2H), 2.22 (dd, J=15.2, 7.7 Hz, 1H), 2.09-1.96 (m, 3H), 1.90-1.62 (m, 6H).

### Example 27: tert-butyl 2-(4,6-dimethoxypyrimidine-5-carboxamido)-7-(trifluoromethyl)spiro[chromeno[4,3-d]thiazole-4,4′-piperidine]-1′-carboxylate

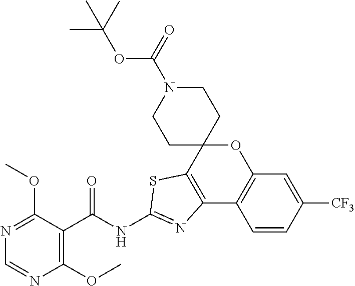 [SMILES: COc1ncnc(OC)c1C(=O)Nc1nc2c(s1)C1(CCN(C(=O)OC(C)(C)C)CC1)Oc1cc(C(F)(F)F)ccc1-2]

### Example 27A: tert-butyl 4-oxo-7-(trifluoromethyl)spiro[chromane-2,4′-piperidine]-1′-carboxylate

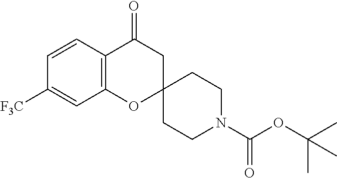 [SMILES: CC(C)(C)OC(=O)N1CCC2(CC1)CC(=O)c1ccc(C(F)(F)F)cc1O2]

At 0° C., 1-(2-hydroxy-4-(trifluoromethyl)phenyl)ethan-1-one (150.0 mg, 0.735 mmol) was dissolved in methanol (3 mL), tert-butyl 4-oxopiperidine-1-carboxylate (146.5 mg, 0.735 mmol) and tetrahydropyrrole (104.4 mg, 1.471 mmol) were added, and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE:EA=15:1)) to afford the title compound (240 mg, 84.8%) as a yellow oil. ¹H NMR (400 MHz, Chloroform-d) δ 7.98-7.94 (m, 1H), 7.27 (d, J=1.6 Hz, 1H), 7.23 (d, J=1.6 Hz, 1H), 3.88 (s, 2H), 3.21 (s, 1H), 2.76 (s, 2H), 2.03 (s, 1H), 1.45 (s, 9H).

### Example 27B: tert-butyl 2-amino-7-(trifluoromethyl)spiro[chromeno[4,3-d]thiazole-4,4′-piperidine]-1′-carboxylate

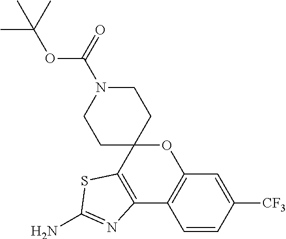 [SMILES: CC(C)(C)OC(=O)N1CCC2(CC1)Oc1cc(C(F)(F)F)ccc1-c1nc(N)sc12]

Into a 10 mL round-bottom flask were added tert-butyl 4-oxo-7-(trifluoromethyl)spiro[chromane-2,4′-piperidine]-1′-carboxylate (280 mg, 0.727 mmol, 1.00 equiv.), hexane (5 mL), molecular sieve, pyrrolidine (155 mg, 2.18 mmol, 3.00 equiv.), and TsOH·H₂O (10 mg, 0.073 mmol, 0.10 equiv.) at room temperature. The resulting mixture was stirred for 3 h at 80° C. under argon atmosphere. The resulting mixture was cooled down to room temperature, then concentrated under reduced pressure. To the above mixture was added MeOH (2 mL), S (58 mg, 1.81 mmol, 2.50 equiv.) and NH₂CN (150 mg, 1.81 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc (4:1)) to afford the title compound (70 mg, 22% yield) as a light yellow solid. ESI-MS m/z=442.15 [M+H]⁺; Calculated MW: 441.13

### Example 27C: tert-butyl 2-(4,6-dimethoxypyrimidine-5-carboxamido)-7-(trifluoromethyl)spiro[chromeno[4,3-d]thiazole-4,4′-piperidine]-1′-carboxylate

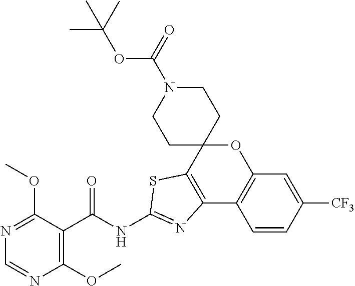 [SMILES: COc1ncnc(OC)c1C(=O)Nc1nc2c(s1)C1(CCN(C(=O)OC(C)(C)C)CC1)Oc1cc(C(F)(F)F)ccc1-2]

To a stirred solution of 4,6-dimethoxypyrimidine-5-carboxylic acid (32.0 mg, 0.175 mmol, 1.10 equiv.) in DCM (1.0 mL) was added (COCl)₂ (101.0 mg, 0.794 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL), then added to a stirred solution of tert-butyl 2-amino-7-(trifluoromethyl)spiro[chromeno[4,3-d]thiazole-4,4′-piperidine]-1′-carboxylate (70.0 mg, 0.159 mmol, 1.00 equiv.), TEA (160.0 mg, 1.587 mmol, 10.00 equiv.) and DMAP (20.0 mg, 0.159 mmol, 1.00 equiv.) in DCM (1.0 mL) in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (DCM/PE/EtOAc (7:4:1)) to afford the title compound (7 mg, 7% yield) as an off-white solid. ESI-MS m/z=608.24 [M+H]⁺; Calculated MW: 607.17; ¹H NMR (400 MHz, Chloroform-d) δ 10.78 (s, 1H), 8.37 (d, J=1.0 Hz, 1H), 7.65 (d, J=7.8 Hz, 1H), 7.19 (d, J=9.0 Hz, 2H), 4.02 (d, J=1.0 Hz, 6H), 3.31 (s, 4H), 2.27-2.16 (m, 2H), 1.87 (td, J=13.1, 4.8 Hz, 2H), 1.49 (d, J=0.9 Hz, 9H).

### Example 28: N—(4-(but-3-en-1-yl)-7-(trifluoromethyl)-4H-chromeno[4,3-d]thiazol-2-yl)-4,6-dimethoxypyrimidine-5-carboxamide

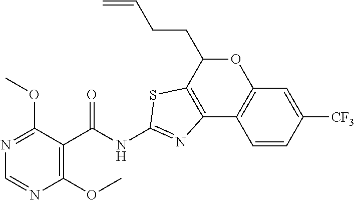 [SMILES: C=CCCC1Oc2cc(C(F)(F)F)ccc2-c2nc(NC(=O)c3c(OC)ncnc3OC)sc21]

### Example 28A: 2-(but-3-en-1-yl)-7-(trifluoromethyl) chroman-4-one

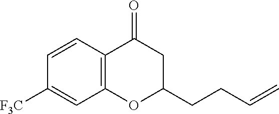 [SMILES: C=CCCC1CC(=O)c2ccc(C(F)(F)F)cc2O1]

At 0° C., 1-(2-hydroxy-4-(trifluoromethyl)phenyl)ethan-1-one (300 mg, 1.471 mmol) was dissolved in methanol (3 mL), added to pent-4-enal (123.7 mg, 2.941 mmol), tetrahydropyrrole (208.8 mg, 2.941 mmol), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (100 mg, 25.1%) as a yellow oil. ¹H NMR (400 MHz, Chloroform-d) δ 7.97 (d, J=8.1 Hz, 1H), 7.26 (s, 1H), 7.22 (s, 0H), 5.83 (ddt, J=17.0, 10.2, 6.7 Hz, 1H), 5.14-4.97 (m, 2H), 4.51 (ddd, J=14.2, 8.5, 6.4 Hz, 1H), 2.76-2.71 (m, 2H), 2.30 (dp, J=21.4, 7.3 Hz, 2H), 2.01 (dtd, J=14.3, 8.1, 5.9 Hz, 1H), 1.87-1.78 (m, 1H).

### Example 28B: 4-(but-3-en-1-yl)-7-(trifluoromethyl)-4H-chromeno[4,3-d]thiazol-2-amine

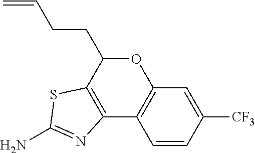 [SMILES: C=CCCC1Oc2cc(C(F)(F)F)ccc2-c2nc(N)sc21]

Into a 10 mL round-bottom flask were added 2-(but-3-en-1-yl)-7-(trifluoromethyl) chroman-4-one (190 mg, 0.704 mmol, 1.00 equiv.), hexane (5 mL), molecular sieve, pyrrolidine (250 mg, 3.52 mmol, 5.00 equiv.), and TsOH·H₂O (14 mg, 0.07 mmol, 0.10 equiv.) at room temperature. The resulting mixture was stirred for 3 h at 80° C. under argon atmosphere. The resulting mixture was cooled down to room temperature, then concentrated under reduced pressure. To the above mixture was added MeOH (2 mL), S (56 mg, 1.76 mmol, 2.50 equiv.) and NH₂CN (145 mg, 1.76 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc=3:1) to afford the title compound (30 mg, 13% yield) as a light yellow oil. ESI-MS m/z=327.08 [M+H]⁺; Calculated MW: 326.07

### Example 58C: N—(4-(but-3-en-1-yl)-7-(trifluoromethyl)-4H-chromeno[4,3-d]thiazol-2-yl)-4,6-dimethoxypyrimidine-5-carboxamide

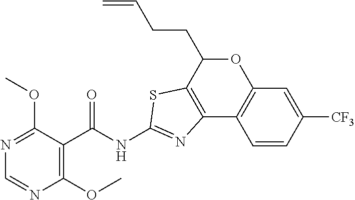 [SMILES: C=CCCC1Oc2cc(C(F)(F)F)ccc2-c2nc(NC(=O)c3c(OC)ncnc3OC)sc21]

To a stirred solution of 4,6-dimethoxypyrimidine-5-carboxylic acid (19.0 mg, 0.101 mmol, 1.10 equiv.) in DCM (1.0 mL) was added (COCl)₂ (60.0 mg, 0.460 mmol, 5.0 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL), then added to a stirred solution of 4-(but-3-en-1-yl)-7-(trifluoromethyl)-4H-chromeno[4,3-d]thiazol-2-amine (30.0 mg, 0.092 mmol, 1.0 equiv.), TEA (93.0 mg, 0.92 mmol, 10.0 equiv.) and DMAP (11.0 mg, 0.009 mmol, 1.0 equiv.) in DCM (1.00 mL) in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (DCM/PE/EtOAc (7:4:1)) to afford the title compound (7.6 mg, 17% yield) as an off-white solid. ESI-MS m/z=493.14 [M+H]⁺; Calculated MW: 492.11; 1H NMR (400 MHz, Chloroform-d) δ 11.47 (s, 1H), 8.24 (s, 1H), 7.53-7.48 (m, 1H), 7.15 (d, J=1.7 Hz, 1H), 7.10 (ddd, J=8.0, 1.8, 0.9 Hz, 1H), 5.86 (ddt, J=16.9, 10.2, 6.6 Hz, 1H), 5.62 (dd, J=7.8, 4.5 Hz, 1H), 5.16-5.02 (m, 2H), 3.93 (s, 6H), 2.36 (tdt, J=8.5, 7.0, 1.9 Hz, 2H), 2.17-2.08 (m, 1H), 2.05-1.94 (m, 1H).

### Example 29: 4,6-dimethoxy-N—(4-phenethyl-7-(trifluoromethyl)-4H-chromeno[4,3-d]thiazol-2-yl)pyrimidine-5-carboxamide

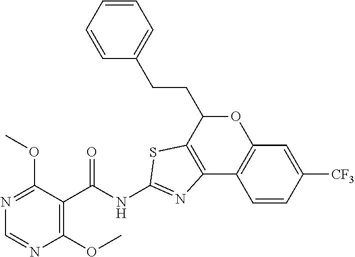 [SMILES: COc1ncnc(OC)c1C(=O)Nc1nc2c(s1)C(CCc1ccccc1)Oc1cc(C(F)(F)F)ccc1-2]

### Example 29A: 2-phenethyl-7-(trifluoromethyl) chroman-4-one

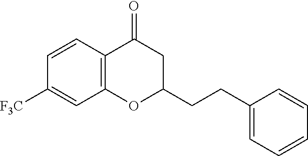 [SMILES: O=C1CC(CCc2ccccc2)Oc2cc(C(F)(F)F)ccc21]

At 0° C., 1-(2-hydroxy-4-(trifluoromethyl)phenyl)ethan-1-one (300 mg, 1.471 mmol) was dissolved in methanol (3 mL), added to 3-phenylpropanal (197.3 mg, 2.941 mmol), tetrahydropyrrole (208.8 mg, 2.941 mmol), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE:EA=30:1) to afford the title compound (110 mg, 23.4%) as a yellow oil. ¹H NMR (400 MHz, Chloroform-d) δ 7.97 (d, J=8.1 Hz, 1H), 7.33-7.27 (m, 3H), 7.24-7.18 (m, 4H), 4.47 (s, 0H), 2.87 (dd, J=25.8, 8.5 Hz, 1H), 2.77-2.72 (m, 2H), 2.24 (d, J=8.5 Hz, OH), 2.03 (s, 1H).

### Example 29B: 4-phenethyl-7-(trifluoromethyl)-4H-chromeno[4,3-d]thiazol-2-amine

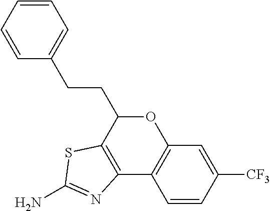 [SMILES: Nc1nc2c(s1)C(CCc1ccccc1)Oc1cc(C(F)(F)F)ccc1-2]

Into a 10 mL round-bottom flask were added 2-phenethyl-7-(trifluoromethyl) chroman-4-one (110 mg, 0.344 mmol, 1.00 equiv.), hexane (5 mL), molecular sieve, pyrrolidine (122 mg, 1.72 mmol, 5.00 equiv.), and TsOH·H₂O (6.5 mg, 0.034 mmol, 0.10 equiv.) at room temperature. The resulting mixture was stirred for 3 h at 80° C. under argon atmosphere, cooled down to room temperature, then concentrated under reduced pressure. To the above mixture was added MeOH (2 mL), S (28 mg, 0.859 mmol, 2.50 equiv.) and NH₂CN (71 mg, 0.859 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc=3:1) to afford the title compound (22 mg, 17% yield) as a light yellow oil. ESI-MS m/z=377.12 [M+H]⁺; Calculated MW: 376.09

### Example 29C: 4,6-dimethoxy-N—(4-phenethyl-7-(trifluoromethyl)-4H-chromeno[4,3-d]thiazol-2-yl)pyrimidine-5-carboxamide

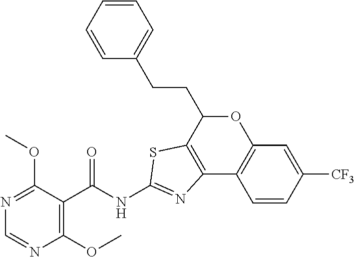 [SMILES: COc1ncnc(OC)c1C(=O)Nc1nc2c(s1)C(CCc1ccccc1)Oc1cc(C(F)(F)F)ccc1-2]

To a stirred solution of 4,6-dimethoxypyrimidine-5-carboxylic acid (12.0 mg, 0.062 mmol, 1.10 equiv.) in DCM (1.00 mL) was added (COCl)₂ (37.0 mg, 0.293 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL), then added to a stirred solution of 4-phenethyl-7-(trifluoromethyl)-4H-chromeno[4,3-d]thiazol-2-amine (22.0 mg, 0.058 mmol, 1.00 equiv.), TEA (59.0 mg, 0.585 mmol, 10.00 equiv.) and DMAP (7.0 mg, 0.058 mmol, 1.00 equiv.) in DCM (1.00 mL) in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (DCM/PE/EtOAc (7:4:1)) to afford the title compound (2.5 mg, 7.8% yield) as an off-white solid. ESI-MS m/z=543.15 [M+H]⁺; Calculated MW: 542.12; 1H NMR (400 MHz, Chloroform-d) δ 8.41 (s, 1H), 7.66 (d, J=7.9 Hz, 1H), 7.33-7.27 (m, 2H), 7.24-7.15 (m, 5H), 5.60 (dd, J=7.9, 4.3 Hz, 1H), 4.06 (s, 6H), 2.96-2.89 (m, 2H), 2.43-2.16 (m, 2H).

### Example 30: N—(4′-(benzyloxy)-7-(trifluoromethyl)spiro[chromeno[4,3-d]thiazole-4,1′-cyclohexan]-2-yl)-4,6-dimethoxypyrimidine-5-carboxamide

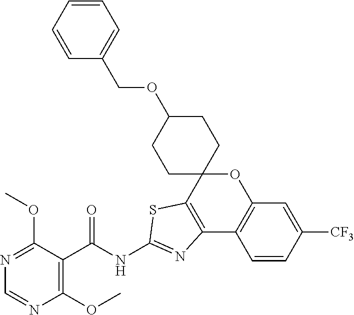 [SMILES: COc1ncnc(OC)c1C(=O)Nc1nc2c(s1)C1(CCC(OCc3ccccc3)CC1)Oc1cc(C(F)(F)F)ccc1-2]

### Example 30A: 4′-(benzyloxy)-7-(trifluoromethyl)spiro[chromane-2,1′-cyclohexan]-4-one

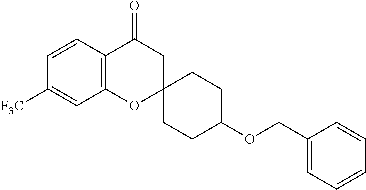 [SMILES: O=C1CC2(CCC(OCc3ccccc3)CC2)Oc2cc(C(F)(F)F)ccc21]

At 0° C., 1-(2-hydroxy-4-(trifluoromethyl)phenyl)ethan-1-one (200 mg, 0.981 mmol) was dissolved in methanol (3 mL), added to 4-(benzyloxy)cyclohexan-1-one (200.26 mg, 0.981 mmol), tetrahydropyrrole (139.2 mg, 1.961 mmol), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (150 mg, 39.2%) as a yellow oil. ¹H NMR (400 MHz, Chloroform-d) δ 7.97-7.93 (m, 1H), 7.33 (d, J=5.8 Hz, 3H), 7.26-7.20 (m, 3H), 4.50 (s, 2H), 3.71 (s, 1H), 2.75 (s, 2H), 1.89-1.79 (m, 8H).

### Example 30B: 4′-(benzyloxy)-7-(trifluoromethyl)spiro[chromeno[4,3-d]thiazole-4,1′-cyclohexan]-2-amine

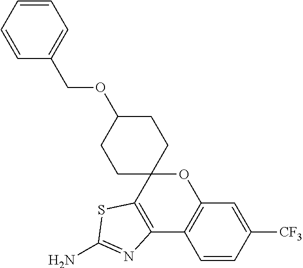 [SMILES: Nc1nc2c(s1)C1(CCC(OCc3ccccc3)CC1)Oc1cc(C(F)(F)F)ccc1-2]

Into a 10 mL round-bottom flask were added 4′-(benzyloxy)-7-(trifluoromethyl)spiro[chromane-2,1′-cyclohexan]-4-one (120 mg, 0.307 mmol, 1.00 equiv.), hexane (5 mL), molecular sieve, pyrrolidine (109 mg, 1.538 mmol, 5.00 equiv.), and TsOH·H₂O (6 mg, 0.030 mmol, 0.10 equiv.) at room temperature. The resulting mixture was stirred for 3 h at 80° C. under argon atmosphere. The resulting mixture was cooled down to room temperature, then concentrated under reduced pressure. To the above mixture was added MeOH (2 mL), S (25 mg, 0.769 mmol, 2.50 equiv.) and NH₂CN (64 mg, 0.769 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×15 mL). The combined organic layers were washed with brine (2×10 mL), dried over anhydrous Na₂SO₄. After filtration, the filtrate was concentrated under reduced pressure. The residue was purified by prep-TLC (PE/EtOAc=3:1) to afford the title compound (18 mg, 13% yield) as a yellow brown oil. ESI-MS m/z=447.15 [M+H]⁺; Calculated MW: 446.13

### Example 30C: N—(4′-(benzyloxy)-7-(trifluoromethyl)spiro[chromeno[4,3-d]thiazole-4,1′-cyclohexan]-2-yl)-4,6-dimethoxypyrimidine-5-carboxamide

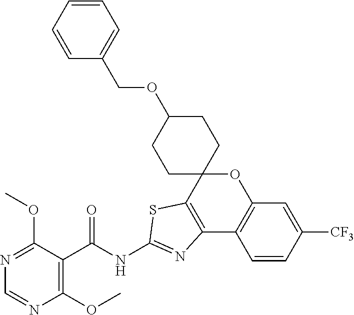 [SMILES: COc1ncnc(OC)c1C(=O)Nc1nc2c(s1)C1(CCC(OCc3ccccc3)CC1)Oc1cc(C(F)(F)F)ccc1-2]

To a stirred solution of 4,6-dimethoxypyrimidine-5-carboxylic acid (4.00 mg, 0.022 mmol, 1.10 equiv.) in DCM (1.00 mL) was added (COCl)₂ (13.00 mg, 0.101 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4′-(benzyloxy)-7-(trifluoromethyl)spiro[chromeno[4,3-d]thiazole-4,1′-cyclohexan]-2-amine (9.00 mg, 0.020 mmol, 1.00 equiv.) in THF (1.00 mL) was added NaH (3.00 mg, 0.06 mmol, 3.00 equiv., 60%) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (3 mg, 24% yield) as an off-white solid. ESI-MS m/z=613.23 [M+H]⁺; Calculated MW: 612.17; ¹H NMR (400 MHz, Chloroform-d) δ 11.20 (s, 1H), 8.30 (s, 1H), 7.64-7.50 (m, 1H), 7.41-7.33 (m, 4H), 7.32-7.26 (m, 1H), 7.18-7.11 (m, 2H), 4.54 (d, J=3.9 Hz, 2H), 3.96 (s, 6H), 3.78 (s, 1H), 2.38-2.18 (m, 1H), 2.16-2.06 (m, 3H), 2.05-1.88 (m, 4H).

### Example 31: N—(4′-ethyl-7-(trifluoromethyl)spiro[chromeno[4,3-d]thiazole-4,1′-cyclohexan]-2-yl)-4,6-dimethoxypyrimidine-5-carboxamide

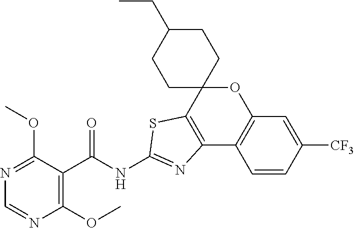 [SMILES: CCC1CCC2(CC1)Oc1cc(C(F)(F)F)ccc1-c1nc(NC(=O)c3c(OC)ncnc3OC)sc12]

### Example 31A: 4′-ethyl-7-(trifluoromethyl)spiro[chromane-2,1′-cyclohexan]-4-one

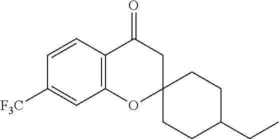 [SMILES: CCC1CCC2(CC1)CC(=O)c1ccc(C(F)(F)F)cc1O2]

At 0° C., 1-(2-hydroxy-4-(trifluoromethyl)phenyl)ethan-1-one (200 mg, 0.981 mmol) was dissolved in methanol (3 mL), added to 4-ethylcyclohexan-1-one (123.7 mg, 0.981 mmol), tetrahydropyrrole (139.2 mg, 1.961 mmol), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (200 mg, 65.5%) as a yellow oil. ¹H NMR (400 MHz, Chloroform-d) δ 7.94 (d, J=8.2 Hz, 1H), 7.25 (s, 1H), 7.19 (dd, J=8.2, 1.6 Hz, 1H), 2.69 (s, 1H), 1.56 (s, 3H), 1.39 (d, J=2.8 Hz, OH), 1.33-1.23 (m, 2H).

### Example 31B: 4′-ethyl-7-(trifluoromethyl)spiro[chromeno[4,3-d]thiazole-4,1′-cyclohexan]-2-amine

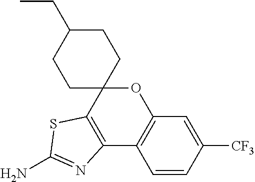 [SMILES: CCC1CCC2(CC1)Oc1cc(C(F)(F)F)ccc1-c1nc(N)sc12]

4′-ethyl-7-(trifluoromethyl)spiro[chromane-2,1′-cyclohexan]-4-one (200 mg, 0.640 mmol) was dissolved in ethanol (3 mL) at room temperature and thiourea (97.3 mg, 1.281 mmol), I₂ (195.2 mg, 0.768 mmol), triethylamine (6.5 mg, 0.064 mmol) were added. The resulting mixture was stirred overnight at 85° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=5:1) to afford the title compound (19 mg, 93%) as a brown solid. ESI-MS m/z=369.12 [M+H]⁺; Calculated MW: 368.42.

### Example 31C: N—(4′-ethyl-7-(trifluoromethyl)spiro[chromeno[4,3-d]thiazole-4,1′-cyclohexan]-2-yl)-4,6-dimethoxypyrimidine-5-carboxamide

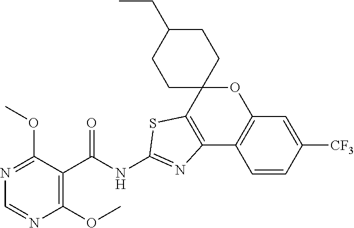 [SMILES: CCC1CCC2(CC1)Oc1cc(C(F)(F)F)ccc1-c1nc(NC(=O)c3c(OC)ncnc3OC)sc12]

To a stirred solution of 4,6-dimethoxypyrimidine-5-carboxylic acid (11.0 mg, 0.057 mmol, 1.10 equiv.) in DCM (1.00 mL) was added (COCl)₂ (38.0 mg, 0.258 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4′-ethyl-7-(trifluoromethyl)spiro[chromeno[4,3-d]thiazole-4,1′-cyclohexan]-2-amine (19.0 mg, 0.052 mmol, 1.00 equiv.) in THF (1.00 mL) was added NaH (6.5 mg, 0.155 mmol, 3.00 equiv., 60%) in portions at 0° C. The resulting mixture was stirred for 0.5 h at 0° C. under argon atmosphere. To the above mixture was added the acyl chloride/THE dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (4.8 mg, 98% yield) as a light yellow solid. ESI-MS m/z=535.18 [M+H]⁺; Calculated MW: 534.15; ¹H NMR (400 MHz, Chloroform-d) δ 11.40 (s, 1H), 8.27 (d, J=4.6 Hz, 1H), 7.54 (t, J=8.2 Hz, 1H), 7.20-7.15 (m, 1H), 7.10 (dd, J=8.4, 3.4 Hz, 1H), 3.94 (dd, J=4.3, 1.3 Hz, 6H), 2.37-2.27 (m, 2H), 2.05-1.96 (m, 1H), 1.82-1.62 (m, 5H), 1.56-1.44 (m, 1H), 1.30-1.26 (m, 2H), 0.94 (t, J=7.4 Hz, 3H).

### Example 32: ethyl 2-(4,6-dimethoxypyrimidine-5-carboxamido)-7-(trifluoromethyl)spiro[chromeno[4,3-d]thiazole-4,1′-cyclohexane]-4′-carboxylate

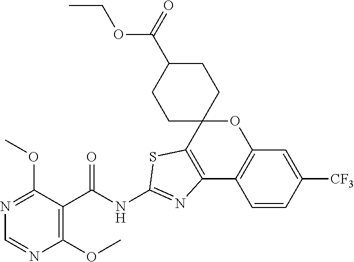 [SMILES: CCOC(=O)C1CCC2(CC1)Oc1cc(C(F)(F)F)ccc1-c1nc(NC(=O)c3c(OC)ncnc3OC)sc12]

### Example 32A: ethyl 4-oxo-7-(trifluoromethyl)spiro[chromane-2,1′-cyclohexane]-4′-carboxylate

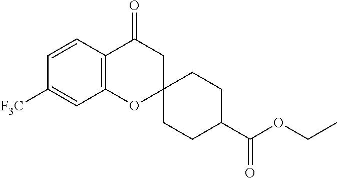 [SMILES: CCOC(=O)C1CCC2(CC1)CC(=O)c1ccc(C(F)(F)F)cc1O2]

To a stirred solution of 1-(2-hydroxy-4-(trifluoromethyl)phenyl)ethan-1-one (200 mg, 0.980 mmol, 1.00 equiv.) and ethyl 4-oxocyclohexanecarboxylate (170 mg, 0.980 mmol, 1.00 equiv.) in MeOH (2 mL) was added pyrrolidine (139 mg, 1.961 mmol, 2.00 equiv.) at 0° C. The resulting mixture was stirred overnight at room temperature, extracted with EA (2×20 mL) and the combined organic layers were washed with brine (1×10 mL), dried over anhydrous Na₂SO₄. After filtration, the filtrate was concentrated under reduced pressure. The crude product was purified by prep-TLC (PE:EA=30:1) to afford the title compound (230 mg, 66%) as yellow oil. ¹H NMR (400 MHz, Chloroform-d) δ 7.94 (dd, J=8.1, 3.8 Hz, 1H), 7.31-7.27 (m, 1H), 7.23-7.19 (m, 1H), 4.22-4.04 (m, 2H), 2.75 (d, J=31.3 Hz, 2H), 2.41-2.13 (m, 3H), 1.94-1.74 (m, 6H), 1.28-1.24 (m, 3H).

### Example 32B: ethyl 2-amino-7-(trifluoromethyl)spiro[chromeno[4,3-d]thiazole-4,1′-cyclohexane]-4′-carboxylate

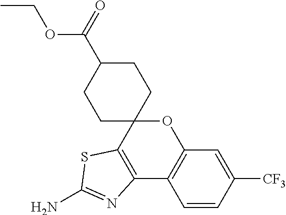 [SMILES: CCOC(=O)C1CCC2(CC1)Oc1cc(C(F)(F)F)ccc1-c1nc(N)sc12]

To a suspension of ethyl 4-oxo-7-(trifluoromethyl)spiro[chromane-2,1′-cyclohexane]-4′-carboxylate (130 mg, 0.365 mmol, 1.00 equiv.) and TsOH-H₂O (7 mg, 0.0365 mmol, 0.10 equiv.) in n-hexane (5 mL), pyrrolidine (130 mg, 1.826 mmol, 5.00 equiv.) was added at room temperature, and the mixture was stirred for 2 h at 80° C. Then the reaction mixture was concentrated in vacuo and dissolved in MeOH (2 mL). S (29 mg, 0.913 mmol, 2.5 equiv.) was added at 0° C. and the mixture was stirred for 5 mins at 0° C. Then 51% w/w % cyanamide (75 mg, 0.913 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (22 mg, 15%) as a yellow solid. ESI-MS m/z=413.13 [M+H]⁺; Calculated MW: 412.10.

### Example 32C: ethyl 2-(4,6-dimethoxypyrimidine-5-carboxamido)-7-(trifluoromethyl)spiro[chromeno[4,3-d]thiazole-4,1′-cyclohexane]-4′-carboxylate

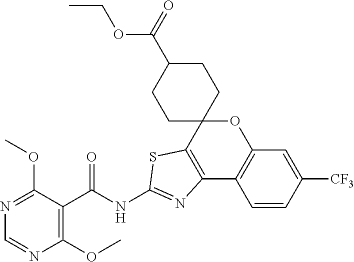 [SMILES: CCOC(=O)C1CCC2(CC1)Oc1cc(C(F)(F)F)ccc1-c1nc(NC(=O)c3c(OC)ncnc3OC)sc12]

To a suspension of 4,6-dimethoxypyrimidine-5-carboxylic acid (12 mg, 0.064 mmol, 1.20 equiv.) in CH₂Cl₂ (0.5 mL) and catalytic amount of DMF, oxalyl chloride (14 mg, 0.107 mmol, 2.0 equiv.) was added dropwise at 0° C., and the mixture was stirred for 3 h at room temperature. Then the reaction mixture was concentrated in vacuo and dissolved in CH₂Cl₂ (0.5 mL), ethyl 2-amino-7-(trifluoromethyl)spiro[chromeno[4,3-d]thiazole-4,1′-cyclohexane]-4′-carboxylate (22 mg, 0.053 mmol, 1.0 equiv.), TEA (22 mg, 0.214 mmol, 4.0 equiv.) and DMAP (0.65 mg, 0.0053 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (7.3 mg, 24%) as a light yellow solid. ESI-MS m/z=579.22 [M+H]⁺; Calculated MW: 578.14. ¹H NMR (400 MHz, Chloroform-d) δ 11.38 (s, 1H), 8.26 (d, J=1.3 Hz, 1H), 7.53 (d, J=7.9 Hz, 1H), 7.22 (d, J=1.8 Hz, 1H), 7.11 (d, J=8.0 Hz, 1H), 4.18 (qd, J=7.1, 1.3 Hz, 2H), 3.93 (d, J=1.2 Hz, 6H), 2.47-2.34 (m, 2H), 2.12-1.92 (m, 5H), 1.71 (td, J=13.7, 4.4 Hz, 2H), 1.29 (td, J=7.2, 1.3 Hz, 3H).

### Example 33: 4,6-dimethoxy-N—(4′-methyl-7-(trifluoromethyl)spiro[chromeno[4,3-d]thiazole-4,1′-cyclohexan]-2-yl)pyrimidine-5-carboxamide

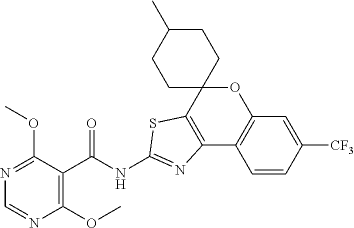 [SMILES: COc1ncnc(OC)c1C(=O)Nc1nc2c(s1)C1(CCC(C)CC1)Oc1cc(C(F)(F)F)ccc1-2]

### Example 33A: 4′-methyl-7-(trifluoromethyl)spiro[chromane-2,1′-cyclohexan]-4-one

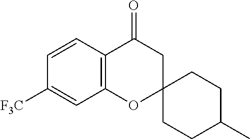 [SMILES: CC1CCC2(CC1)CC(=O)c1ccc(C(F)(F)F)cc1O2]

To a stirred solution of 1-(2-hydroxy-4-(trifluoromethyl)phenyl)ethan-1-one (200 mg, 0.980 mmol, 1.00 equiv.) and 4-methylcyclohexanone (110 mg, 0.980 mmol, 1.00 equiv.) in MeOH (2 mL) was added pyrrolidine (139 mg, 1.961 mmol, 2.00 equiv.) at 0° C. The resulting mixture was stirred overnight at room temperature, extracted with EA (2×20 mL) and the combined organic layers were washed with brine (1×10 mL), dried over anhydrous Na₂SO₄. After filtration, the filtrate was concentrated under reduced pressure. The crude product was purified by prep-TLC (PE) to afford the title compound (175 mg, 59.9%) as a yellow solid. ¹H NMR (400 MHz, Chloroform-d) δ 7.94 (dp, J=8.1, 0.8 Hz, 1H), 7.26 (dt, J=1.5, 0.8 Hz, 1H), 7.19 (ddd, J=8.1, 1.6, 0.7 Hz, 1H), 2.69 (s, 2H), 2.09 (d, J=10.9 Hz, 2H), 1.53 (d, J=8.4 Hz, 2H), 1.44-1.34 (m, 5H), 0.98-0.93 (m, 3H).

### Example 33B: 4′-methyl-7-(trifluoromethyl)spiro[chromeno[4,3-d]thiazole-4,1′-cyclohexan]-2-amine

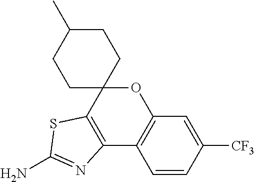 [SMILES: CC1CCC2(CC1)Oc1cc(C(F)(F)F)ccc1-c1nc(N)sc12]

To a suspension of 4′-methyl-7-(trifluoromethyl)spiro[chromane-2,1′-cyclohexan]-4-one (140 mg, 0.470 mmol, 1.00 equiv.) and TsOH-H₂O (9 mg, 0.0470 mmol, 0.10 equiv.) in n-hexane (5 mL), pyrrolidine (167 mg, 2.349 mmol, 5.00 equiv.) was added at room temperature, and the mixture was stirred for 2 h at 80° C. Then the reaction mixture was concentrated in vacuo and dissolved in MeOH (2 mL), S (38 mg, 1.174 mmol, 2.5 equiv.) was added at 0° C. and the mixture was stirred for 5 mins at 0° C. Then 51% w/w % cyanamide (97 mg, 1.174 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (12 mg, 7.2%) as a yellow solid. ESI-MS m/z=355.12 [M+H]⁺; Calculated MW: 354.10

### Example 33C: 4,6-dimethoxy-N—(4′-methyl-7-(trifluoromethyl)spiro[chromeno[4,3-d]thiazole-4,1′-cyclohexan]-2-yl)pyrimidine-5-carboxamide

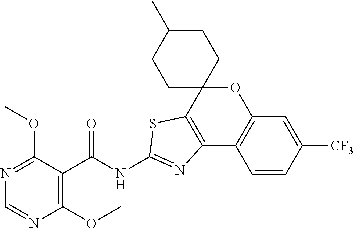 [SMILES: COc1ncnc(OC)c1C(=O)Nc1nc2c(s1)C1(CCC(C)CC1)Oc1cc(C(F)(F)F)ccc1-2]

To a stirred solution of 4′-methyl-7-(trifluoromethyl)spiro[chromeno[4,3-d]thiazole-4,1′-cyclohexan]-2-amine (12 mg, 0.034 mmol, 1.00 equiv.), 4,6-dimethoxypyrimidine-5-carboxylic acid (16 mg, 0.085 mmol, 2.50 equiv.) and NMI (14 mg, 0.169 mmol, 5.00 equiv.) in ACN (0.2 mL) and DMF (0.2 mL) was added TCFH (24 mg, 0.085 mmol, 2.50 equiv.) at 0° C. The resulting mixture was stirred overnight at room temperature. Then the resulting mixture was extracted with EA (2×10 mL). The combined organic layers were washed with sat. NaHCO₃ (1×5 mL) and brine (1×5 mL), dried over anhydrous Na₂SO₄. After filtration, the filtrate was concentrated under reduced pressure. The crude product was purified by prep-TLC (PE:EA=4:1) to afford the title compound (6.3 mg, 36%) as a yellow solid. ESI-MS m/z=521.20 [M+H]⁺; Calculated MW: 520.14. ¹H NMR (400 MHz, Chloroform-d) § 11.21 (s, 1H), 8.29 (s, 1H), 7.55 (dd, J=7.9, 0.9 Hz, 1H), 7.19 (d, J=1.7 Hz, 1H), 7.12 (ddd, J=7.9, 1.7, 0.8 Hz, 1H), 3.95 (s, 6H), 2.37-2.23 (m, 3H), 1.68 (t, J=13.0 Hz, 3H), 1.54-1.46 (m, 3H), 1.01 (d, J=5.5 Hz, 3H).

### Example 34: N—(4′,4′-dimethyl-7-(trifluoromethyl)spiro[chromeno[4,3-d]thiazole-4,1′-cyclohexan]-2-yl)-4,6-dimethoxypyrimidine-5-carboxamide

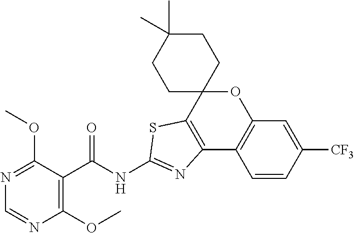 [SMILES: COc1ncnc(OC)c1C(=O)Nc1nc2c(s1)C1(CCC(C)(C)CC1)Oc1cc(C(F)(F)F)ccc1-2]

### Example 34A: 4′,4′-dimethyl-7-(trifluoromethyl)spiro[chromane-2,1′-cyclohexan]-4-one

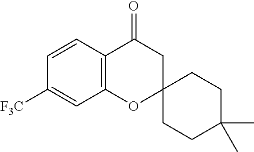 [SMILES: CC1(C)CCC2(CC1)CC(=O)c1ccc(C(F)(F)F)cc1O2]

At 0° C., 1-(2-hydroxy-4-(trifluoromethyl)phenyl)ethan-1-one (200 mg, 0.981 mmol) was dissolved in methanol (3 mL), added to 4,4-dimethylcyclohexan-1-one (123.7 mg, 0.981 mmol), tetrahydropyrrole (139.2 mg, 1.961 mmol), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (256 mg, 83.9%) as a yellow oil. ¹H NMR (400 MHz, Chloroform-d) δ 7.93 (d, J=8.1 Hz, 1H), 7.25 (s, 2H), 7.19 (dd, J=8.2, 1.6 Hz, 1H), 2.73 (s, 2H), 1.90 (d, J=2.8 Hz, 1H), 1.61 (d, J=2.8 Hz, 1H), 1.27-1.22 (m, 3H), 0.98 (s, 3H), 0.92 (s, 3H).

### Example 34B: 4′,4′-dimethyl-7-(trifluoromethyl)spiro[chromeno[4,3-d]thiazole-4,1′-cyclohexan]-2-amine

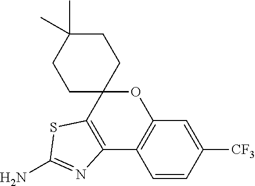 [SMILES: CC1(C)CCC2(CC1)Oc1cc(C(F)(F)F)ccc1-c1nc(N)sc12]

To a flask was added 4′,4′-dimethyl-7-(trifluoromethyl)spiro[chromane-2,1′-cyclohexan]-4-one (256 mg, 0.819 mmol), n-hexane (3 mL), p-toluenesulfonic acid monohydrate (15.6 mg, 0.082 mmol), tetrahydropyrrole (290.9 mg, 4.098 mmol) and 4A molecular sieve. The mixture was stirred under refluxing for 2 hours, cooled to rt and concentrated under reduced pressure. After dissolved in methanol (3 mL), sulfur (65.6 mg, 2.050 mmol) was added at 0° C. and the mixture was stirred for 5 minutes. Subsequently cyanamide (86.1 mg, 2.050 mmol) was added and the reaction was stirred overnight at room temperature and then concentrated. The mixture was diluted with EtOAc and washed with water. The organic phase was washed with brine (1×10 mL), dried over anhydrous Na₂SO₄. After filtration, the filtrate was concentrated under reduced pressure. The crude product was purified by silica gel column chromatography (PE:EA=5:1) to afford the title compound (10 mg, 92%) as a brown solid. ESI-MS m/z=369.12 [M+H]⁺; Calculated MW: 368.42.

### Example 34C: N—(4′,4′-dimethyl-7-(trifluoromethyl)spiro[chromeno[4,3-d]thiazole-4,1′-cyclohexan]-2-yl)-4,6-dimethoxypyrimidine-5-carboxamide

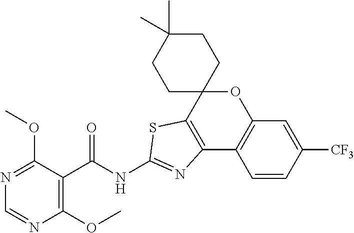 [SMILES: COc1ncnc(OC)c1C(=O)Nc1nc2c(s1)C1(CCC(C)(C)CC1)Oc1cc(C(F)(F)F)ccc1-2]

To a stirred solution of 4,6-dimethoxypyrimidine-5-carboxylic acid (6.0 mg, 0.030 mmol, 1.10 equiv.) in DCM (1.00 mL) was added (COCl)₂ (17.0 mg, 0.136 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4′,4′-dimethyl-7-(trifluoromethyl)spiro[chromeno[4,3-d]thiazole-4,1′-cyclohexan]-2-amine (10.0 mg, 0.027 mmol, 1.00 equiv.) in THF (1.0 mL) was added NaH (4.0 mg, 0.081 mmol, 3.00 equiv., 60%) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (3.7 mg, 25% yield) as an off-white solid. ESI-MS m/z=535.19 [M+H]⁺; Calculated MW: 534.15; ¹H NMR (400 MHz, Chloroform-d) δ 11.15 (s, 1H), 8.30 (s, 1H), 7.56 (d, J=7.9 Hz, 1H), 7.18 (d, J=1.7 Hz, 1H), 7.14-7.09 (m, 1H), 3.96 (s, 6H), 2.19-2.09 (m, 3H), 2.05-1.96 (m, 1H), 1.85 (td, J=13.1, 3.7 Hz, 2H), 1.75 (td, J=12.9, 3.6 Hz, 2H), 1.03 (s, 3H), 0.99 (s, 3H).

### Example 35: N—(3′,3′-dimethyl-7-(trifluoromethyl)spiro[chromeno[4,3-d]thiazole-4,1′-cyclohexan]-2-yl)-4,6-dimethoxypyrimidine-5-carboxamide

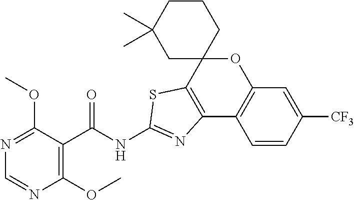 [SMILES: COc1ncnc(OC)c1C(=O)Nc1nc2c(s1)C1(CCCC(C)(C)C1)Oc1cc(C(F)(F)F)ccc1-2]

### Example 35A: 3′,3′-dimethyl-7-(trifluoromethyl)spiro[chromane-2,1′-cyclohexan]-4-one

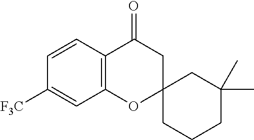 [SMILES: CC1(C)CCCC2(CC(=O)c3ccc(C(F)(F)F)cc3O2)C1]

At 0° C., 1-(2-hydroxy-4-(trifluoromethyl)phenyl)ethan-1-one (200 mg, 0.981 mmol) was dissolved in methanol (3 mL), added to 3,3-dimethylcyclohexan-1-one (123.7 mg, 0.981 mmol), tetrahydropyrrole (139.2 mg, 1.961 mmol), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (256 mg, 83.9%) as a yellow oil. ¹H NMR (400 MHz, Chloroform-d) δ 7.94 (dq, J=7.7, 0.9 Hz, OH), 7.21-7.17 (m, 1H), 2.74-2.59 (m, 1H), 2.13-2.02 (m, 1H), 1.95-1.81 (m, 1H), 1.38-1.31 (m, OH), 1.31-1.13 (m, 2H), 1.04 (s, 1H), 0.88 (s, 1H).

### Example 35B: 3′,3′-dimethyl-7-(trifluoromethyl)spiro[chromeno[4,3-d]thiazole-4,1′-cyclohexan]-2-amine

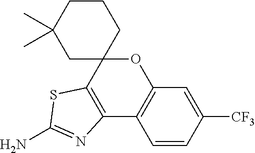 [SMILES: CC1(C)CCCC2(C1)Oc1cc(C(F)(F)F)ccc1-c1nc(N)sc12]

To a flask was added 3′,3′-dimethyl-7-(trifluoromethyl)spiro[chromane-2,1′-cyclohexan]-4-one (216 mg, 0.691 mmol), n-hexane (4 mL) at room temperature, p-toluenesulfonic acid monohydrate (13.2 mg, 0.069 mmol), tetrahydropyrrole (245.5 mg, 3.458 mmol) and 4A molecular sieve. The mixture was stirred under refluxing for 2 hours, cooled to rt and concentrated under reduced pressure. After dissolved in methanol (4 mL), sulfur (55.4 mg, 1.727 mmol) was added at 0° C. and the mixture was stirred for 5 minutes. Subsequently cyanamide (72.7 mg, 1.727 mmol) was added, and the reaction was stirred overnight at room temperature and then concentrated. The mixture was diluted with EtOAc and washed with water. The organic phase was washed with brine (1×10 mL), dried over anhydrous Na₂SO₄. After filtration, the filtrate was concentrated under reduced pressure. The crude product was purified by silica gel column chromatography (PE:EA=5:1) to afford the title compound (10 mg, 95%) as a brown solid. ESI-MS m/z=369.12 [M+H]⁺; Calculated MW: 368.42.

### Example 35C: N—(3′,3′-dimethyl-7-(trifluoromethyl)spiro[chromeno[4,3-d]thiazole-4,1′-cyclohexan]-2-yl)-4,6-dimethoxypyrimidine-5-carboxamide

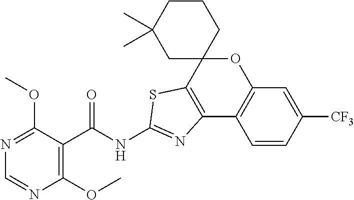 [SMILES: COc1ncnc(OC)c1C(=O)Nc1nc2c(s1)C1(CCCC(C)(C)C1)Oc1cc(C(F)(F)F)ccc1-2]

To a stirred solution of 4,6-dimethoxypyrimidine-5-carboxylic acid (6.0 mg, 0.030 mmol, 1.10 equiv.) in DCM (1.00 mL) was added (COCl)₂ (17.0 mg, 0.136 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 3′,3′-dimethyl-7-(trifluoromethyl)spiro[chromeno[4,3-d]thiazole-4,1′-cyclohexan]-2-amine (10.0 mg, 0.027 mmol, 1.00 equiv.) in THF (1.00 mL) was added NaH (4.0 mg, 0.081 mmol, 3.00 equiv., 60%) in portions at 0° C. The resulting mixture was stirred for 0.5 h at 0° C. under argon atmosphere. To the above mixture was added the acyl chloride/THE dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (6.6 mg, 45% yield) as an off-white solid. ESI-MS m/z=535.19 [M+H]⁺; Calculated MW: 534.15; ¹H NMR (400 MHz, Chloroform-d) δ 11.51 (s, 1H), 8.22 (s, 1H), 7.49 (d, J=7.8 Hz, 1H), 7.14-7.07 (m, 2H), 3.90 (s, 6H), 2.38 (d, J=13.6 Hz, 1H), 2.10-1.96 (m, 2H), 1.62 (d, J=10.6 Hz, 5H), 1.19 (s, 3H), 0.91 (s, 3H).

### Example 36: 4,6-dimethoxy-N—(4′-(p-tolyl)-7-(trifluoromethyl)spiro[chromeno[4,3-d]thiazole-4,1′-cyclohexan]-2-yl)pyrimidine-5-carboxamide

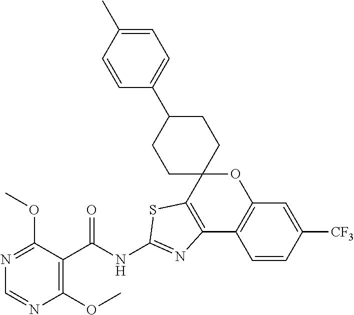 [SMILES: COc1ncnc(OC)c1C(=O)Nc1nc2c(s1)C1(CCC(c3ccc(C)cc3)CC1)Oc1cc(C(F)(F)F)ccc1-2]

### Example 36A: 4′-(p-tolyl)-7-(trifluoromethyl)spiro[chromane-2,1′-cyclohexan]-4-one

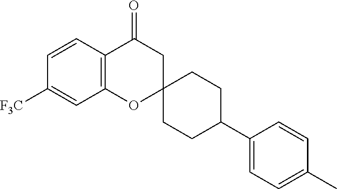 [SMILES: Cc1ccc(C2CCC3(CC2)CC(=O)c2ccc(C(F)(F)F)cc2O3)cc1]

To a stirred solution of 1-(2-hydroxy-4-(trifluoromethyl)phenyl)ethan-1-one (200 mg, 0.980 mmol, 1.00 equiv.) and 4-oxo-1-phenylcyclohexanecarbonitrile (184 mg, 0.980 mmol, 1.00 equiv.) in MeOH (2 mL) was added pyrrolidine (139 mg, 1.961 mmol, 2.00 equiv.) at 0° C. The precipitated product was filtered, washed with MeOH and dried under vacuum to afford the title compound as white solid (230 mg, 62.7%). ¹H NMR (400 MHz, Chloroform-d) δ 7.41 (d, J=7.9 Hz, 1H), 7.19-7.09 (m, 6H), 3.02-2.99 (m, 3H), 2.32 (s, 3H), 2.23-2.10 (m, 2H), 1.91 (dq, J=6.5, 3.4 Hz, 6H).

### Example 36B: 4′-(p-tolyl)-7-(trifluoromethyl)spiro[chromeno[4,3-d]thiazole-4,1′-cyclohexan]-2-amine

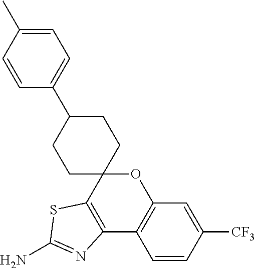 [SMILES: Cc1ccc(C2CCC3(CC2)Oc2cc(C(F)(F)F)ccc2-c2nc(N)sc23)cc1]

To a suspension of 4′-(p-tolyl)-7-(trifluoromethyl)spiro[chromane-2,1′-cyclohexan]-4-one (230 mg, 0.615 mmol, 1.00 equiv.) and TsOH-H₂O (12 mg, 0.0614 mmol, 0.10 equiv.) in n-hexane (5 mL), pyrrolidine (218 mg, 3.075 mmol, 5.00 equiv.) was added at room temperature, and the mixture was stirred for 2 h at 80° C. Then the reaction mixture was concentrated in vacuo and dissolved in MeOH (2 mL), S (49 mg, 1.537 mmol, 2.5 equiv.) was added at 0° C. and the mixture was stirred for 5 mins at 0° C. Then 51% w/w % cyanamide (127 mg, 1.537 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=3:1) to afford the title compound (160 mg, 61%) as a white solid. ESI-MS m/z=430.13 [M+H]⁺; Calculated MW: 430.13

### Example 36C: 4,6-dimethoxy-N—(4′-(p-tolyl)-7-(trifluoromethyl)spiro[chromeno[4,3-d]thiazole-4,1′-cyclohexan]-2-yl)pyrimidine-5-carboxamide

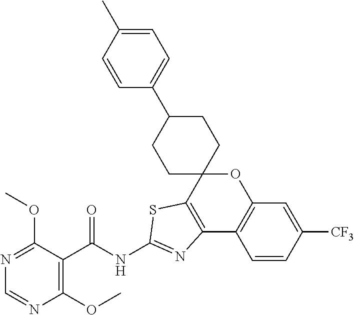 [SMILES: COc1ncnc(OC)c1C(=O)Nc1nc2c(s1)C1(CCC(c3ccc(C)cc3)CC1)Oc1cc(C(F)(F)F)ccc1-2]

To a suspension of 4,6-dimethoxypyrimidine-5-carboxylic acid (8.4 mg, 0.046 mmol, 1.20 equiv.) in CH₂Cl₂ (1 mL) and catalytic amount of DMF, oxalyl chloride (9.6 mg, 0.076 mmol, 2.0 equiv.) was added dropwise at 0° C., and the mixture was stirred for 3 h at room temperature. After concentration, the mixture was dissolved in CH₂Cl₂ (1 mL), 4′-(p-tolyl)-7-(trifluoromethyl)spiro[chromeno[4,3-d]thiazole-4,1′-cyclohexan]-2-amine (16.4 mg, 0.038 mmol, 1.0 equiv.), TEA (15.3 mg, 0.152 mmol, 4.0 equiv.) and DMAP (0.46 mg, 0.0038 mmol, 0.1 equiv.) were added, and the mixture was stirred overnight at room temperature. Then the resulting mixture was extracted with EA (2×5 mL) and washed with sat. H₂O (1×5 mL). The combined organic layers were washed with brine (1×5 mL), dried over anhydrous Na₂SO₄. After filtration, the filtrate was concentrated under reduced pressure. The crude product was purified by prep-TLC (PE:EA=3:1) to afford the title compound (4.1 mg, 96%) as a white solid. ESI-MS m/z=597.27 [M+H]⁺; Calculated MW: 596.63. ¹H NMR (400 MHz, Chloroform-d) δ 8.39 (s, 1H), 7.65 (d, J=7.9 Hz, 1H), 7.23-7.13 (m, 6H), 4.04 (s, 7H), 2.62 (s, 1H), 2.45 (d, J=14.4 Hz, 2H), 2.34 (s, 4H), 2.25-2.17 (m, 2H), 1.67-1.60 (m, 1H).

### Example 37: N—(3′,5′-dimethyl-7-(trifluoromethyl)spiro[chromeno[4,3-d]thiazole-4,1′-cyclohexan]-2-yl)-4,6-dimethoxypyrimidine-5-carboxamide

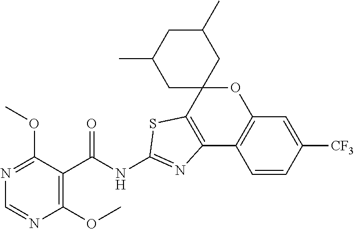 [SMILES: COc1ncnc(OC)c1C(=O)Nc1nc2c(s1)C1(CC(C)CC(C)C1)Oc1cc(C(F)(F)F)ccc1-2]

### Example 37A: 3′,5′-dimethyl-7-(trifluoromethyl)spiro[chromane-2,1′-cyclohexan]-4-one

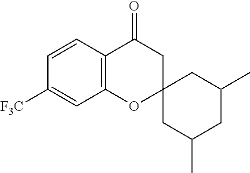 [SMILES: CC1CC(C)CC2(CC(=O)c3ccc(C(F)(F)F)cc3O2)C1]

At 0° C., 1-(2-hydroxy-4-(trifluoromethyl)phenyl)ethan-1-one (200 mg, 0.981 mmol) was dissolved in methanol (3 mL), added to 3,5-dimethylcyclohexan-1-one (123.7 mg, 0.981 mmol), tetrahydropyrrole (139.2 mg, 1.961 mmol), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (253 mg, 82.9%) as a yellow oil. ¹H NMR (400 MHz, Chloroform-d) δ 7.94 (dq, J=8.7, 0.9 Hz, 1H), 7.19 (ddd, J=6.2, 1.7, 0.9 Hz, 2H), 2.69 (s, 2H), 2.07-1.98 (m, 3H), 1.95-1.83 (m, 1H), 1.25-1.23 (m, 1H), 0.87 (d, J=6.6 Hz, 5H).

### Example 37B: 3′,5′-dimethyl-7-(trifluoromethyl)spiro[chromeno[4,3-d]thiazole-4,1′-cyclohexan]-2-amine

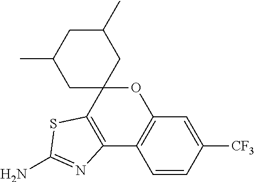 [SMILES: CC1CC(C)CC2(C1)Oc1cc(C(F)(F)F)ccc1-c1nc(N)sc12]

3′,5′-dimethyl-7-(trifluoromethyl)spiro[chromane-2,1′-cyclohexan]-4-one (253 mg, 0.810 mmol) was dissolved in ethanol (3 mL) at room temperature and thiourea (123.2 mg, 1.620 mmol), I₂ (246.9 mg, 0.972 mmol), triethylamine (8.2 mg, 0.081 mmol) were added. The resulting mixture was stirred overnight at 80° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=5:1) to afford the title compound (14 mg, 70%) as a brown solid. ESI-MS m/z=369.12 [M+H]⁺; Calculated MW: 368.42.

### Example 37C: N—(3′,5′-dimethyl-7-(trifluoromethyl)spiro[chromeno[4,3-d]thiazole-4,1′-cyclohexan]-2-yl)-4,6-dimethoxypyrimidine-5-carboxamide

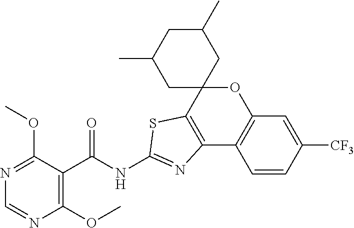 [SMILES: COc1ncnc(OC)c1C(=O)Nc1nc2c(s1)C1(CC(C)CC(C)C1)Oc1cc(C(F)(F)F)ccc1-2]

To a stirred solution of 4,6-dimethoxypyrimidine-5-carboxylic acid (5.0 mg, 0.027 mmol, 1.10 equiv.) in DCM (1.00 mL) was added (COCl)₂ (16.0 mg, 0.122 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 3′,5′-dimethyl-7-(trifluoromethyl)spiro[chromeno[4,3-d]thiazole-4,1′-cyclohexan]-2-amine (9.0 mg, 0.024 mmol, 1.00 equiv.) in THF (1.0 mL) was added NaH (3.0 mg, 0.073 mmol, 3.00 equiv., 60%) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (3.5 mg, 26% yield) as an off-white solid. ESI-MS m/z=535.19 [M+H]⁺; Calculated MW: 534.15; ¹H NMR (400 MHz, Chloroform-d) δ 11.05 (d, J=19.5 Hz, 1H), 8.32 (d, J=4.4 Hz, 1H), 7.67-7.55 (m, 1H), 7.13 (td, J=4.4, 1.7 Hz, 2H), 3.98 (d, J=5.8 Hz, 6H), 2.31-2.17 (m, 3H), 2.03 (s, 2H), 1.93-1.72 (m, 2H), 1.48-1.37 (m, 1H), 0.93 (dd, J=7.8, 6.5 Hz, 6H).

### Example 38: N—(3′-ethyl-7-(trifluoromethyl)spiro[chromeno[4,3-d]thiazole-4,1′-cyclohexan]-2-yl)-4,6-dimethoxypyrimidine-5-carboxamide

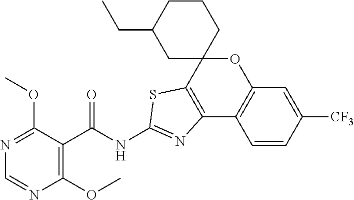 [SMILES: CCC1CCCC2(C1)Oc1cc(C(F)(F)F)ccc1-c1nc(NC(=O)c3c(OC)ncnc3OC)sc12]

### Example 38A: 3′-ethyl-7-(trifluoromethyl)spiro[chromane-2,1′-cyclohexan]-4-one

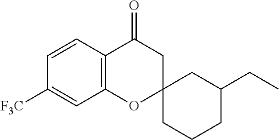 [SMILES: CCC1CCCC2(CC(=O)c3ccc(C(F)(F)F)cc3O2)C1]

At 0° C., 1-(2-hydroxy-4-(trifluoromethyl)phenyl)ethan-1-one (200 mg, 0.981 mmol, 1 equiv.) was dissolved in methanol (3 mL), added to 3-ethylcyclohexan-1-one (123.7 mg, 0.981 mmol, 1 equiv.), tetrahydropyrrole (139.2 mg, 1.961 mmol, 2 equiv.), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (268 mg, 87.8%) as a yellow oil. ¹H NMR (400 MHz, Chloroform-d) δ 7.93 (ddd, J=9.4, 8.3, 0.9 Hz, 2H), 7.24-7.16 (m, 3H), 2.89 (s, 1H), 2.75-2.63 (m, 3H), 2.12-2.02 (m, 5H), 1.88-1.59 (m, 3H), 1.24 (tdd, J=14.4, 8.6, 6.9 Hz, 8H), 0.85 (t, J=7.5 Hz, 7H).

### Example 38B: 3′-ethyl-7-(trifluoromethyl)spiro[chromeno[4,3-d]thiazole-4,1′-cyclohexan]-2-amine

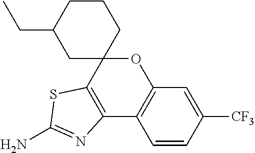 [SMILES: CCC1CCCC2(C1)Oc1cc(C(F)(F)F)ccc1-c1nc(N)sc12]

3′-ethyl-7-(trifluoromethyl)spiro[chromane-2,1′-cyclohexan]-4-one (268 mg, 0.858 mmol, 1 equiv.) was dissolved in ethanol (3 mL) at room temperature and thiourea (130.4 mg, 1.716 mmol, 2 equiv.), I₂ (261.5 mg, 1.030 mmol, 1.2equiv.), triethylamine (8.7 mg, 0.086 mmol, 0.1 equiv.) were added. The resulting mixture was stirred overnight at 80° C. Then it was cooled to room temperature and the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by silica gel column chromatography (PE:EA=5:1) to afford the title compound (16 mg, 71%) as a brown solid. ESI-MS m/z=369.12 [M+H]⁺; Calculated MW: 368.42.

### Example 38C: N—(3′-ethyl-7-(trifluoromethyl)spiro[chromeno[4,3-d]thiazole-4,1′-cyclohexan]-2-yl)-4,6-dimethoxypyrimidine-5-carboxamide

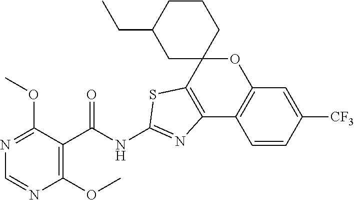 [SMILES: CCC1CCCC2(C1)Oc1cc(C(F)(F)F)ccc1-c1nc(NC(=O)c3c(OC)ncnc3OC)sc12]

To a stirred solution of 4,6-dimethoxypyrimidine-5-carboxylic acid (5.0 mg, 0.027 mmol, 1.10 equiv.) in DCM (1.00 mL) was added (COCl)₂ (16.0 mg, 0.122 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 3′-ethyl-7-(trifluoromethyl)spiro[chromeno[4,3-d]thiazole-4,1′-cyclohexan]-2-amine (9.0 mg, 0.024 mmol, 1.00 equiv.) in THF (1.00 mL) was added NaH (3.0 mg, 0.073 mmol, 3.00 equiv., 60%) in portions at 0° C. The resulting mixture was stirred for 0.5 h at 0° C. under argon atmosphere. To the above mixture was added the acyl chloride/THE dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (3.5 mg, 26% yield) as an off-white solid. ESI-MS m/z=535.21 [M+H]⁺; Calculated MW: 534.15; ¹H NMR (400 MHz, Chloroform-d) δ 11.45 (s, 1H), 8.24 (d, J=1.8 Hz, 1H), 7.52 (dd, J=13.7, 7.9 Hz, 1H), 7.15 (dd, J=10.2, 1.7 Hz, 1H), 7.12-7.06 (m, 1H), 3.92 (d, J=4.2 Hz, 6H), 2.38-2.18 (m, 3H), 2.00 (q, J=6.7, 6.2 Hz, 1H), 1.93-1.76 (m, 1H), 1.73-1.46 (m, 6H), 0.93-0.87 (m, 3H).

### Example 39: benzyl(2-(4,6-dimethoxypyrimidine-5-carboxamido)-7-(trifluoromethyl)spiro[chromeno[4,3-d]thiazole-4,1′-cyclohexan]-3′-yl)carbamate

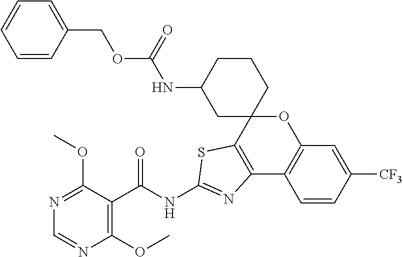 [SMILES: COc1ncnc(OC)c1C(=O)Nc1nc2c(s1)C1(CCCC(NC(=O)OCc3ccccc3)C1)Oc1cc(C(F)(F)F)ccc1-2]

### Example 39A: benzyl (4-oxo-7-(trifluoromethyl)spiro[chromane-2,1′-cyclohexan]-3′-yl)carbamate

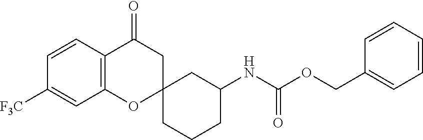 [SMILES: O=C(NC1CCCC2(CC(=O)c3ccc(C(F)(F)F)cc3O2)C1)OCc1ccccc1]

At 0° C., 1-(2-hydroxy-4-(trifluoromethyl)phenyl)ethan-1-one (200 mg, 0.981 mmol, 1 equiv.) was dissolved in methanol (3 mL), added to benzyl (3-oxocyclohexyl)carbamate (242.4 mg, 0.981 mmol, 1 equiv.), tetrahydropyrrole (139.2 mg, 1.961 mmol, 2 equiv.), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE:EA=5:1) to afford the title compound (356 mg, 83.9%) as a yellow oil. ¹H NMR (400 MHz, Chloroform-d) δ 7.93 (d, J=8.3 Hz, 1H), 7.35-7.30 (m, 5H), 7.25 (s, 1H), 7.23-7.20 (m, 1H), 5.06 (s, 2H), 3.97 (s, 1H), 2.81-2.63 (m, 2H), 2.16-2.04 (m, 2H), 1.32-1.20 (m, 5H).

### Example 39B: benzyl(2-amino-7-(trifluoromethyl)spiro[chromeno[4,3-d]thiazole-4,1′-cyclohexan]-3′-yl)carbamate

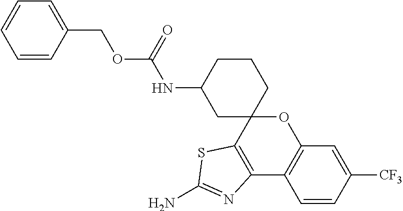 [SMILES: Nc1nc2c(s1)C1(CCCC(NC(=O)OCc3ccccc3)C1)Oc1cc(C(F)(F)F)ccc1-2]

To a flask was added benzyl (4-oxo-7-(trifluoromethyl)spiro[chromane-2,1′-cyclohexan]-3′-yl)carbamate (356 mg, 0.727 mmol, 1 equiv.), n-hexane (4 mL), p-toluenesulfonic acid monohydrate (13.8 mg, 0.073 mmol, 0.1 equiv.), tetrahydropyrrole (258.2 mg, 3.636 mmol, 5 equiv.) and 4A molecular sieve. The mixture was stirred under refluxing for 2 hours, cooled to rt and concentrated under reduced pressure. After dissolved in methanol (4 mL), sulfur (58.2 mg, 1.818 mmol, 2.5 equiv.) was added at 0° C. and the mixture was stirred for 5 minutes. Subsequently cyanamide (76.4 mg, 1.818 mmol, 2.5 equiv.) was added and the reaction was stirred overnight at room temperature and then concentrated. The mixture was diluted with EtOAc and washed with water. The organic phase was washed with brine (1×10 mL), dried over anhydrous Na₂SO₄. After filtration, the filtrate was concentrated under reduced pressure. The crude product was purified by silica gel column chromatography (PE:EA=3:1) to afford the title compound (147 mg, 99%) as a brown solid. ESI-MS m/z=490.15 [M+H]⁺; Calculated MW: 489.51.

### Example 39C: benzyl(2-(4,6-dimethoxypyrimidine-5-carboxamido)-7-(trifluoromethyl)spiro[chromeno[4,3-d]thiazole-4,1′-cyclohexan]-3′-yl)carbamate

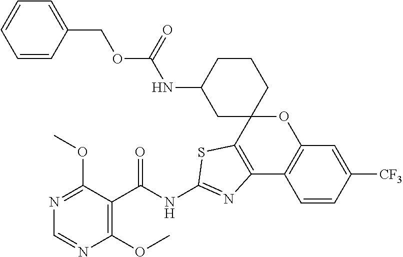 [SMILES: COc1ncnc(OC)c1C(=O)Nc1nc2c(s1)C1(CCCC(NC(=O)OCc3ccccc3)C1)Oc1cc(C(F)(F)F)ccc1-2]

To a suspension of 4,6-dimethoxypyrimidine-5-carboxylic acid (8.4 mg, 0.046 mmol, 1.20 equiv.) in CH₂Cl₂ (1 mL) and catalytic amount of DMF, oxalyl chloride (9.6 mg, 0.076 mmol, 2.0 equiv.) was added dropwise at 0° C., and the mixture was stirred for 3 h at room temperature. After concentration, the mixture was dissolved in THF (1.5 mL) for use. benzyl(2-amino-7-(trifluoromethyl)spiro[chromeno[4,3-d]thiazole-4,1′-cyclohexan]-3′-yl)carbamate (30 mg, 0.061 mmol, 1.00 equiv.) was dissolved in THF and sodium hydride (4.41 mg, 0.184 mmol, 3.00 equiv.) was added at 0° C., and the mixture was stirred overnight at room temperature. Then the resulting mixture was extracted with EA (2×5 mL) and washed with sat. NH₄Cl (1×5 mL). The combined organic layers were washed with brine (1×5 mL), dried over anhydrous Na₂SO₄. After filtration, the filtrate was concentrated under reduced pressure. The crude product was purified by prep-TLC (PE:EA=3:1) to afford the title compound (10 mg, 97%) as a white solid. ESI-MS m/z=656.25 [M+H]⁺; Calculated MW: 655.65. ¹H NMR (400 MHz, Chloroform-d) δ 11.55 (d, J=49.5 Hz, 1H), 8.23 (d, J=10.1 Hz, 1H), 7.51 (dd, J=22.0, 7.9 Hz, 1H), 7.38-7.27 (m, 6H), 7.22-7.05 (m, 2H), 5.10 (d, J=16.3 Hz, 3H), 3.90 (d, J=8.4 Hz, 7H), 2.67 (d, J=13.2 Hz, 1H), 2.37-2.13 (m, 3H), 2.10-1.93 (m, 1H), 1.74 (d, J=16.0 Hz, 1H), 1.56-1.45 (m, 1H).

### Example 40: N—(4′,4′-difluoro-7-(trifluoromethyl)spiro[chromeno[4,3-d]thiazole-4,1′-cyclohexan]-2-yl)-4,6-dimethoxypyrimidine-5-carboxamide

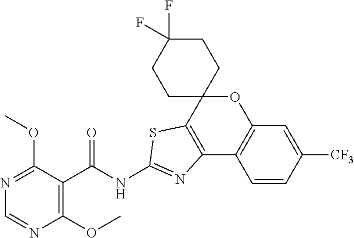 [SMILES: COc1ncnc(OC)c1C(=O)Nc1nc2c(s1)C1(CCC(F)(F)CC1)Oc1cc(C(F)(F)F)ccc1-2]

### Example 40A: 4′,4′-difluoro-7-(trifluoromethyl)spiro[chromane-2,1′-cyclohexan]-4-one

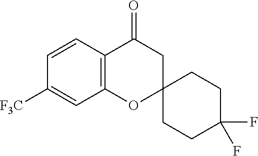 [SMILES: O=C1CC2(CCC(F)(F)CC2)Oc2cc(C(F)(F)F)ccc21]

To a stirred solution of 1-(2-hydroxy-4-(trifluoromethyl)phenyl)ethan-1-one (200 mg, 0.980 mmol, 1.00 equiv.) and 4,4-difluorocyclohexanone (131 mg, 0.980 mmol, 1.00 equiv.) in MeOH (2 mL) was added pyrrolidine (139 mg, 1.961 mmol, 2.00 equiv.) at 0° C. The precipitated product was filtered, washed with MeOH and dried under vacuum to afford the title compound as white solid (150 mg, 48%). ¹H NMR (400 MHz, Chloroform-d) δ 8.01-7.91 (m, 1H), 7.27 (s, 2H), 2.77 (d, J=0.9 Hz, 2H), 2.21-1.97 (m, 6H), 1.83-1.71 (m, 2H).

### Example 40B: 4′,4′-difluoro-7-(trifluoromethyl)spiro[chromeno[4,3-d]thiazole-4,1′-cyclohexan]-2-amine

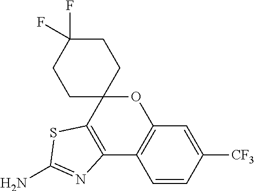 [SMILES: Nc1nc2c(s1)C1(CCC(F)(F)CC1)Oc1cc(C(F)(F)F)ccc1-2]

To a suspension of 4′,4′-difluoro-7-(trifluoromethyl)spiro[chromane-2,1′-cyclohexan]-4-one (150 mg, 0.469 mmol, 1.00 equiv.) and TsOH-H₂O (9 mg, 0.0469 mmol, 0.10 equiv.) in n-hexane (5 mL), pyrrolidine (166 mg, 2.344 mmol, 5.00 equiv.) was added at room temperature, and the mixture was stirred for 2 h at 80° C. Then the reaction mixture was concentrated in vacuo and dissolved in MeOH (2 mL), S (38 mg, 1.172 mmol, 2.5 equiv.) was added at 0° C. and the mixture was stirred for 5 mins at 0° C. Then 51% w/w % cyanamide (96 mg, 1.172 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=3:1) to afford the title compound (36 mg, 20%) as a yellow solid. ESI-MS m/z=377.11 [M+H]⁺; Calculated MW: 376.07

### Example 40C: N—(4′,4′-difluoro-7-(trifluoromethyl)spiro[chromeno[4,3-d]thiazole-4,1′-cyclohexan]-2-yl)-4,6-dimethoxypyrimidine-5-carboxamide

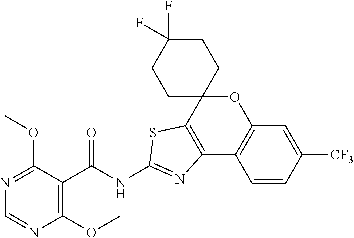 [SMILES: COc1ncnc(OC)c1C(=O)Nc1nc2c(s1)C1(CCC(F)(F)CC1)Oc1cc(C(F)(F)F)ccc1-2]

To a suspension of 4,6-dimethoxypyrimidine-5-carboxylic acid (12 mg, 0.064 mmol, 1.20 equiv.) in CH₂Cl₂ (0.5 mL) and catalytic amount of DMF, oxalyl chloride (14 mg, 0.107 mmol, 2.0 equiv.) was added dropwise at 0° C., and the mixture was stirred for 3 h at room temperature. Then the reaction mixture was concentrated in vacuo and dissolved in CH₂Cl₂ (0.5 mL), 4′,4′-difluoro-7-(trifluoromethyl)spiro[chromeno[4,3-d]thiazole-4,1′-cyclohexan]-2-amine (20 mg, 0.053 mmol, 1.0 equiv.), TEA (22 mg, 0.214 mmol, 4.0 equiv.) and DMAP (0.65 mg, 0.0053 mmol, 0.1 equiv.) were added, and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by prep-TLC (DCM:PE:EA=7:4:1) to afford the title compound (8.2 mg, 28%) as a white solid. ESI-MS m/z=543.17 [M+H]⁺; Calculated MW: 542.10. ¹H NMR (400 MHz, Chloroform-d) δ 11.26 (s, 1H), 8.28 (s, 1H), 7.58 (d, J=7.9 Hz, 1H), 7.22-7.11 (m, 2H), 3.95 (s, 6H), 2.45-2.05 (m, 8H).

### Example 41: N—(4-hexyl-4-methyl-7-(trifluoromethyl)-4H-chromeno[4,3-d]thiazol-2-yl)-4,6-dimethoxypyrimidine-5-carboxamide

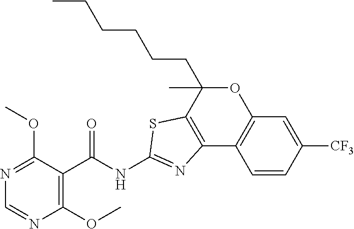 [SMILES: CCCCCCC1(C)Oc2cc(C(F)(F)F)ccc2-c2nc(NC(=O)c3c(OC)ncnc3OC)sc21]

### Example 41A: 2-hexyl-2-methyl-7-(trifluoromethyl) chroman-4-one

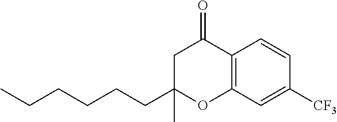 [SMILES: CCCCCCC1(C)CC(=O)c2ccc(C(F)(F)F)cc2O1]

At 0° C., 1-(2-hydroxy-4-(trifluoromethyl)phenyl)ethan-1-one (200 mg, 0.981 mmol, 1 equiv.) was dissolved in methanol (3 mL), added to octan-2-one (125.7 mg, 0.981 mmol, 1 equiv.), tetrahydropyrrole (139.2 mg, 1.961 mmol, 2 equiv.), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (220 mg, 71.6%) as a yellow oil. ¹H NMR (400 MHz, Chloroform-d) δ 7.95 (dq, J=8.5, 0.9 Hz, 1H), 7.30-7.25 (m, 2H), 2.88 (d, J=16.4 Hz, 1H), 2.81-2.71 (m, 3H), 2.13-1.95 (m, 1H), 1.57 (s, 4H), 1.35-1.19 (m, 1H).

### Example 41B: 4-hexyl-4-methyl-7-(trifluoromethyl)-4H-chromeno[4,3-d]thiazol-2-amine

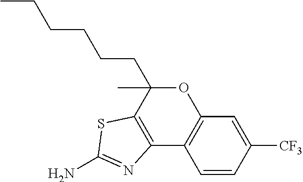 [SMILES: CCCCCCC1(C)Oc2cc(C(F)(F)F)ccc2-c2nc(N)sc21]

To a flask was added 2-hexyl-2-methyl-7-(trifluoromethyl) chroman-4-one (220 mg, 0.700 mmol, 1 equiv.), n-hexane (4 mL), p-toluenesulfonic acid monohydrate (13.3 mg, 0.070 mmol, 0.1 equiv.), tetrahydropyrrole (248.5 mg, 3.500 mmol, 5 equiv.) and 4A molecular sieve. The mixture was stirred under refluxing for 2 hours, cooled to rt and concentrated under reduced pressure. After dissolved in methanol (4 mL), sulfur (56.0 mg, 1.750 mmol, 2.5 equiv.) was added at 0° C. and the mixture was stirred for 5 minutes. Subsequently cyanamide (42.0 mg, 1.750 mmol, 2.5 equiv.) was added and the reaction was stirred overnight at room temperature and then concentrated. The mixture was diluted with EtOAc and washed with water. The organic phase was washed with brine (1×10 mL), dried over anhydrous Na₂SO₄. After filtration, the filtrate was concentrated under reduced pressure. The crude product was purified by silica gel column chromatography (PE:EA=10:1) to afford the title compound (15 mg, 88%) as a brown solid. ESI-MS m/z=371.18 [M+H]⁺; Calculated MW: 370.43.

### Example 41C: N—(4-hexyl-4-methyl-7-(trifluoromethyl)-4H-chromeno[4,3-d]thiazol-2-yl)-4,6-dimethoxypyrimidine-5-carboxamide

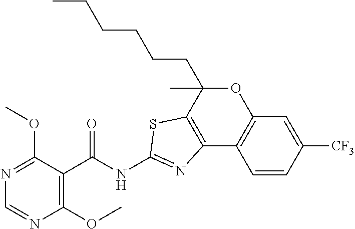 [SMILES: CCCCCCC1(C)Oc2cc(C(F)(F)F)ccc2-c2nc(NC(=O)c3c(OC)ncnc3OC)sc21]

To a suspension of 4,6-dimethoxypyrimidine-5-carboxylic acid (8.9 mg, 0.049 mmol, 1.20 equiv.) in CH₂Cl₂ (1 mL) and catalytic amount of DMF, oxalyl chloride (10.3 mg, 0.081 mmol, 2.0 equiv.) was added dropwise at 0° C., and the mixture was stirred for 3 h at room temperature. After concentration, the mixture was dissolved in THF (1.5 mL) for use. 4-hexyl-4-methyl-7-(trifluoromethyl)-4H-chromeno[4,3-d]thiazol-2-amine (15 mg, 0.041 mmol, 1.00 equiv.) was dissolved in THF and sodium hydride (4.9 mg, 0.121 mmol, 3.00 equiv.) was added at 0° C., and the mixture was stirred overnight at room temperature. Then the resulting mixture was extracted with EA (2×5 mL) and washed with sat. NH₄Cl (1×5 mL). The combined organic layers were washed with brine (1×5 mL), dried over anhydrous Na₂SO₄. After filtration, the filtrate was concentrated under reduced pressure. The crude product was purified by prep-TLC (PE:EA=5:1) to afford the title compound (4.3 mg, 93%) as a white solid. ESI-MS m/z=537.18 [M+H]⁺; Calculated MW: 536.57. ¹H NMR (400 MHz, Chloroform-d) δ 8.40 (s, 1H), 7.63 (d, J=7.9 Hz, 1H), 7.17-7.11 (m, 3H), 4.05 (s, 7H), 2.34 (t, J=7.5 Hz, 1H), 2.25-2.18 (m, 1H), 1.66 (s, 4H), 1.32 (s, 2H), 0.87 (s, 2H).

### Example 42: 4,6-dimethoxy-N—(4-methyl-4-phenethyl-7-(trifluoromethyl)-4H-chromeno[4,3-d]thiazol-2-yl)pyrimidine-5-carboxamide

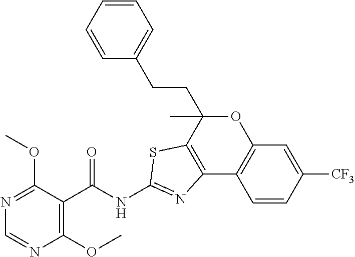 [SMILES: COc1ncnc(OC)c1C(=O)Nc1nc2c(s1)C(C)(CCc1ccccc1)Oc1cc(C(F)(F)F)ccc1-2]

### Example 42A: 2-methyl-2-phenethyl-7-(trifluoromethyl) chroman-4-one

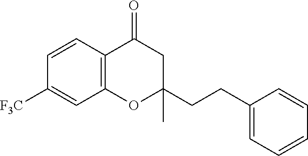 [SMILES: CC1(CCc2ccccc2)CC(=O)c2ccc(C(F)(F)F)cc2O1]

At 0° C., 1-(2-hydroxy-4-(trifluoromethyl)phenyl)ethan-1-one (200 mg, 0.981 mmol, 1 equiv.) was dissolved in methanol (3 mL), added to 4-phenylbutan-2-one (145.3 mg, 0.981 mmol, 1 equiv.), tetrahydropyrrole (139.2 mg, 1.961 mmol, 2 equiv.), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (269 mg, 82.2%) as a yellow oil. ¹H NMR (400 MHz, Chloroform-d) δ 7.93 (dp, J=8.0, 0.9 Hz, 1H), 7.25 (s, 1H), 7.21-7.16 (m, 2H), 2.81 (d, J=16.5 Hz, 1H), 2.67 (d, J=16.5 Hz, 1H), 1.78-1.70 (m, 1H), 1.40 (s, 4H).

### Example 42B: 4-methyl-4-phenethyl-7-(trifluoromethyl)-4H-chromeno[4,3-d]thiazol-2-amine

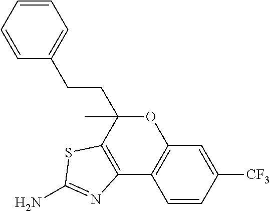 [SMILES: CC1(CCc2ccccc2)Oc2cc(C(F)(F)F)ccc2-c2nc(N)sc21]

To a flask was added 2-methyl-2-phenethyl-7-(trifluoromethyl) chroman-4-one (269 mg, 0.800 mmol, 1 equiv.), n-hexane (4 mL), p-toluenesulfonic acid monohydrate (15.3 mg, 0.080 mmol, 0.1 equiv.), tetrahydropyrrole (248.0 mg, 4.000 mmol, 5 equiv.), and 4A molecular sieve. The mixture was stirred under refluxing for 2 hours, cooled to rt and concentrated under reduced pressure. After dissolved in methanol (4 mL), sulfur (64.0 mg, 2.000 mmol, 2.5 equiv.) was added at 0° C. and the mixture was stirred for 5 minutes. Subsequently cyanamide (84.0 mg, 2.000 mmol, 2.5 equiv.) was added and the reaction was stirred overnight at room temperature and then concentrated. The mixture was diluted with EtOAc and washed with water. The organic phase was washed with brine (1×10 mL), dried over anhydrous Na₂SO₄. After filtration, the filtrate was concentrated under reduced pressure. The crude product was purified by silica gel column chromatography (PE:EA=10:1) to afford the title compound (35 mg, 97.67%) as a brown solid. ESI-MS m/z=391.12 [M+H]⁺; Calculated MW: 390.42.

### Example 42C: 4,6-dimethoxy-N—(4-methyl-4-phenethyl-7-(trifluoromethyl)-4H-chromeno[4,3-d]thiazol-2-yl)pyrimidine-5-carboxamide

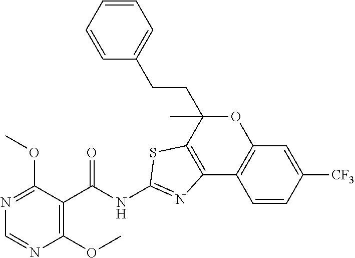 [SMILES: COc1ncnc(OC)c1C(=O)Nc1nc2c(s1)C(C)(CCc1ccccc1)Oc1cc(C(F)(F)F)ccc1-2]

To a suspension of 4,6-dimethoxypyrimidine-5-carboxylic acid (8.9 mg, 0.049 mmol, 1.20 equiv.) in CH₂Cl₂ (1 mL) and catalytic amount of DMF, oxalyl chloride (10.3 mg, 0.081 mmol, 2.0 equiv.) was added dropwise at 0° C., and the mixture was stirred for 3 h at room temperature. After concentration, the mixture was dissolved in THF (1.5 mL) for use. 4-methyl-4-phenethyl-7-(trifluoromethyl)-4H-chromeno[4,3-d]thiazol-2-amine (15 mg, 0.041 mmol, 1.00 equiv.) was dissolved in THF and sodium hydride (4.9 mg, 0.121 mmol, 3.00 equiv.) was added at 0° C., and the mixture was stirred overnight at room temperature. Then the resulting mixture was extracted with EA (2×5 mL) and washed with sat. NH₄Cl (1×5 mL). The combined organic layers were washed with brine (1×5 mL), dried over anhydrous Na₂SO₄. After filtration, the filtrate was concentrated under reduced pressure. The crude product was purified by prep-TLC (PE:EA=5:1) to afford the title compound (4.3 mg, 97%) as a white solid. ESI-MS m/z=557.16 [M+H]⁺; Calculated MW: 556.56. ¹H NMR (400 MHz, Chloroform-d) δ 8.31 (s, 1H), 7.57 (dt, J=8.3, 0.9 Hz, 1H), 7.29-7.25 (m, 2H), 7.20-7.11 (m, 6H), 3.99 (s, 7H), 2.90-2.70 (m, 2H), 2.21 (s, 0H), 1.73 (s, 3H).

### Example 43:4-(but-3-en-1-yloxy)-6-methoxy-N—(7-(trifluoromethyl)spiro[chromeno[4,3-d]thiazole-4, 1′-cyclohexan]-2-yl)pyrimidine-5-carboxamide

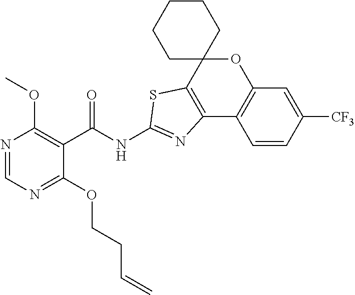 [SMILES: C=CCCOc1ncnc(OC)c1C(=O)Nc1nc2c(s1)C1(CCCCC1)Oc1cc(C(F)(F)F)ccc1-2]

### Example 43A: 4-(but-3-en-1-yloxy)-6-methoxypyrimidine-5-carboxylic acid

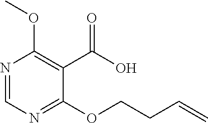 [SMILES: C=CCCOc1ncnc(OC)c1C(=O)O]

To a stirred solution of 3-buten-1-ol (90.0 mg, 1.25 mmol, 1.20 equiv.) in THF (2.00 mL) was added NaH (97.0 mg, 2.29 mmol, 2.20 equiv., 60%) in portions at 0° C. The resulting mixture was stirred for 0.5 h at 0° C. under argon. To the above mixture was added 4,6-dichloropyrimidine-5-carboxylic acid (200 mg, 1.042 mmol, 1.00 equiv.) in THF (1 mL) dropwise at 0° C. The resulting mixture was stirred for additional 16 h at room temperature and monitored by LCMS. Upon the formation of desired intermediate, to the above mixture was added MeONa/MeOH (0.25 mL, 1.25 mmol, 1.20 equiv.) dropwise at 0° C. The resulting mixture was stirred for additional 3 h at room temperature. The reaction was diluted with water, adjusted pH to 5 with HCl (1M), extracted with EtOAc (3×50 mL). The combined organic layers were washed with brine (2×20 mL), dried over anhydrous Na₂SO₄. After filtration, the filtrate was concentrated under reduced pressure. This resulted in the title compound (130 mg, 55% yield) as a light yellow oil. ¹H NMR (400 MHz, Chloroform-d) δ 8.50-8.41 (m, 1H), 5.83 (ddt, J=17.1, 10.2, 6.8 Hz, 1H), 5.21-5.03 (m, 2H), 4.48 (td, J=6.8, 3.7 Hz, 2H), 4.04 (d, J=2.0 Hz, 3H), 2.53 (qt, J=6.8, 1.4 Hz, 2H).

### Example 43B: 4-(but-3-en-1-yloxy)-6-methoxy-N—(7-(trifluoromethyl)spiro[chromeno[4,3-d]thiazole-4,1′-cyclohexan]-2-yl)pyrimidine-5-carboxamide

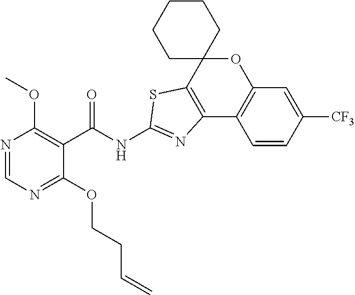 [SMILES: C=CCCOc1ncnc(OC)c1C(=O)Nc1nc2c(s1)C1(CCCCC1)Oc1cc(C(F)(F)F)ccc1-2]

To a stirred solution of 4-(but-3-en-1-yloxy)-6-methoxypyrimidine-5-carboxylic acid (15 mg, 0.067 mmol, 1.00 equiv.) in DCM (1.00 mL) was added (COCl)₂ (42.0 mg, 0.335 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with DCM (1 mL), then added to a stirred solution of 7-(trifluoromethyl)spiro[chromeno[4,3-d]thiazole-4,1′-cyclohexan]-2-amine (15.00 mg, 0.067 mmol, 1.00 equiv.), TEA (68.0 mg, 0.67 mmol, 10.00 equiv.) and DMAP (8.0 mg, 0.067 mmol, 1.00 equiv.) in DCM (1.0 mL) in portions at 0° C. The resulting mixture was stirred for 16 h at room temperature under argon atmosphere. The reaction was diluted with DCM (20 mL), washed with water (2×5 mL), dried over anhydrous Na₂SO₄. After filtration, the filtrate was concentrated under reduced pressure. The residue was purified by prep-TLC (PE/EtOAc/DCM (7:1:4)) to afford the title compound (3.0 mg, 8% yield) as a light yellow solid. ESI-MS m/z=547.25 [M+H]⁺; Calculated MW: 546.15; ¹H NMR (400 MHz, Chloroform-d) δ 10.82 (s, 1H), 8.34 (s, 1H), 7.61 (d, J=7.9 Hz, 1H), 7.19 (d, J=1.7 Hz, 1H), 7.15 (dd, J=7.9, 1.7 Hz, 1H), 5.86 (ddt, J=17.0, 10.2, 6.8 Hz, 1H), 5.27-5.11 (m, 2H), 4.46 (t, J=6.8 Hz, 2H), 4.01 (s, 3H), 2.55 (qt, J=6.8, 1.4 Hz, 2H), 2.37-2.16 (m, 3H), 2.00 (q, J=6.7 Hz, 2H), 1.83 (td, J=11.8, 3.4 Hz, 5H).

### Example 44: N—(4-(but-3-en-1-yl)-4-methyl-7-(trifluoromethyl)-4H-chromeno[4,3-d]thiazol-2-yl)-4,6-dimethoxypyrimidine-5-carboxamide

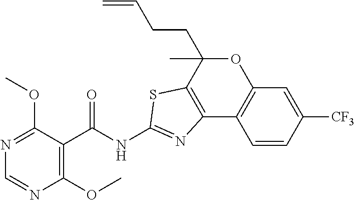 [SMILES: C=CCCC1(C)Oc2cc(C(F)(F)F)ccc2-c2nc(NC(=O)c3c(OC)ncnc3OC)sc21]

### Example 44A: 2-(but-3-en-1-yl)-2-methyl-7-(trifluoromethyl) chroman-4-one

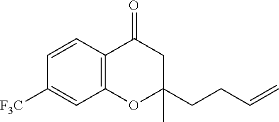 [SMILES: C=CCCC1(C)CC(=O)c2ccc(C(F)(F)F)cc2O1]

At 0° C., 1-(2-hydroxy-4-(trifluoromethyl)phenyl)ethan-1-one (200 mg, 0.981 mmol, 1 equiv.) was dissolved in methanol (3 mL), added to hex-5-en-2-one (96.3 mg, 0.981 mmol, 1 equiv.), tetrahydropyrrole (139.2 mg, 1.961 mmol, 2 equiv.), and the mixture was stirred overnight at room temperature. Then the resulting mixture was extracted with EA (2×10 mL) and washed with H₂O (1×10 mL). The combined organic layers were washed with brine (1×10 mL), dried over anhydrous Na₂SO₄. After filtration, the filtrate was concentrated under reduced pressure. The crude product was purified by prep-TLC (PE=100%) to afford the title compound (160 mg, 57.5%) as a yellow oil. ¹H NMR (400 MHz, Chloroform-d) δ 7.94 (dt, J=7.9, 0.9 Hz, 1H), 7.20 (dt, J=8.7, 0.9 Hz, 2H), 5.79 (ddt, J=16.8, 10.2, 6.5 Hz, 1H), 5.09-4.94 (m, 2H), 2.84 (dd, J=16.5, 0.6 Hz, 1H), 2.69 (d, J=16.5 Hz, 1H), 2.26-2.15 (m, 1H), 1.93-1.74 (m, 1H), 1.42 (s, 3H).

### Example 44B: 4-(but-3-en-1-yl)-4-methyl-7-(trifluoromethyl)-4H-chromeno[4,3-d]thiazol-2-amine

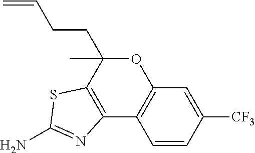 [SMILES: C=CCCC1(C)Oc2cc(C(F)(F)F)ccc2-c2nc(N)sc21]

To a flask was added 2-(but-3-en-1-yl)-2-methyl-7-(trifluoromethyl) chroman-4-one (160 mg, 0.563 mmol, 1 equiv.), n-hexane (4 mL), p-toluenesulfonic acid monohydrate (10.7 mg, 0.056 mmol, 0.1 equiv.), tetrahydropyrrole (199.9 mg, 2.815 mmol, 5 equiv.), and 4A molecular sieve. The mixture was stirred under refluxing for 2 hours, cooled to rt and concentrated under reduced pressure. After dissolved in methanol (4 mL), sulfur (45.1 mg, 1.407 mmol, 2.5 equiv.) was added at 0° C. and the mixture was stirred for 5 minutes. Subsequently cyanamide (59.1 mg, 1.407 mmol, 2.5 equiv.) was added and the reaction was stirred overnight at room temperature and then concentrated. The mixture was diluted with EtOAc and washed with water. The organic phase was washed with brine (1×10 mL), dried over anhydrous Na₂SO₄. After filtration, the filtrate was concentrated under reduced pressure. The crude product was purified by silica gel column chromatography (PE:EA=10:1) to afford the title compound (15 mg, 100%) as a brown solid. ESI-MS m/z=341.07 [M+H]⁺; Calculated MW: 340.36.

### Example 44C: N—(4-(but-3-en-1-yl)-4-methyl-7-(trifluoromethyl)-4H-chromeno[4,3-d]thiazol-2-yl)-4,6-dimethoxypyrimidine-5-carboxamide

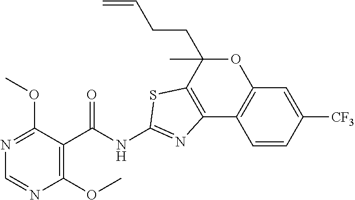 [SMILES: C=CCCC1(C)Oc2cc(C(F)(F)F)ccc2-c2nc(NC(=O)c3c(OC)ncnc3OC)sc21]

To a suspension of 4,6-dimethoxypyrimidine-5-carboxylic acid (9.8 mg, 0.053 mmol, 1.20 equiv.) in CH₂Cl₂ (1 mL) and catalytic amount of DMF, oxalyl chloride (11.2 mg, 0.088 mmol, 2.0 equiv.) was added dropwise at 0° C., and the mixture was stirred for 3 h at room temperature. After concentration, the mixture was dissolved in THF (1.5 mL) for use. 4-(but-3-en-1-yl)-4-methyl-7-(trifluoromethyl)-4H-chromeno[4,3-d]thiazol-2-amine (15 mg, 0.044 mmol, 1.00 equiv.) was dissolved in THF and sodium hydride (3.2 mg, 0.132 mmol, 3.00 equiv.) was added at 0° C., and the mixture was stirred overnight at room temperature. Then the resulting mixture was extracted with EA (2×5 mL) and washed with sat. NH₄Cl (1×5 mL). The combined organic layers were washed with brine (1×5 mL), dried over anhydrous Na₂SO₄. After filtration, the filtrate was concentrated under reduced pressure. The crude product was purified by prep-TLC (PE:EA=5:1) to afford the title compound (10.5 mg, 97%) as a light yellow solid. ESI-MS m/z=507.17 [M+H]⁺; Calculated MW: 506.50. ¹H NMR (400 MHz, Chloroform-d) δ 8.23 (s, 1H), 7.48 (dd, J=7.9, 1.0 Hz, 1H), 7.12 (d, J=1.8 Hz, 1H), 5.86-5.73 (m, 1H), 3.92 (s, 7H), 2.10 (s, 1H), 1.71 (s, 4H).

### Example 45: 4,6-dimethoxy-N—(7-(trifluoromethyl)spiro[chromeno[4,3-d]thiazole-4,4′-piperidin]-2-yl)pyrimidine-5-carboxamide

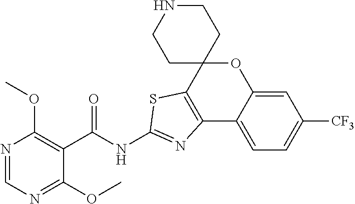 [SMILES: COc1ncnc(OC)c1C(=O)Nc1nc2c(s1)C1(CCNCC1)Oc1cc(C(F)(F)F)ccc1-2]

To a stirred solution of tert-butyl 2-(4,6-dimethoxypyrimidine-5-carboxamido)-7-(trifluoromethyl)spiro[chromeno[4,3-d]thiazole-4,4′-piperidine]-1′-carboxylate (32.00 mg, 0.052 mmol, 1.00 equiv.) in DCM (1.50 mL) was added TFA (0.5 mL) in portions at 0° C. The resulting mixture was stirred for 1 h at room temperature. The reaction was concentrated under reduced pressure. The residue was diluted with water, adjusted pH to 9 with NaHCO₃ (aq.). The resulting mixture was extracted with EtOAc (3×30 mL). The combined organic layers were washed with brine (2×10 mL), dried over anhydrous Na₂SO₄. After filtration, the filtrate was concentrated under reduced pressure. This resulted in the title compound (20 mg, 74% yield) as an off-white solid. ESI-MS m/z=508.20 [M+H]⁺; Calculated MW: 507.12; ¹H NMR (400 MHz, Chloroform-d) δ 10.23 (s, 1H), 8.48 (s, 1H), 7.77 (d, J=7.9 Hz, 1H), 7.32-7.26 (m, 2H), 4.12 (s, 6H), 3.52 (s, 4H), 2.46 (s, 4H).

### Example 46: 4,6-dimethoxy-N—(1′-methyl-7-(trifluoromethyl)spiro[chromeno[4,3-d]thiazole-4,4′-piperidin]-2-yl)pyrimidine-5-carboxamide

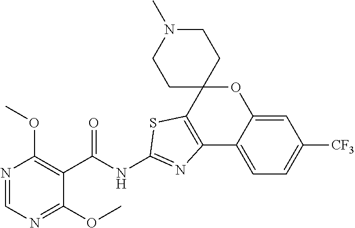 [SMILES: COc1ncnc(OC)c1C(=O)Nc1nc2c(s1)C1(CCN(C)CC1)Oc1cc(C(F)(F)F)ccc1-2]

Into a 10 mL round-bottom flask were added 4,6-dimethoxy-N—(7-(trifluoromethyl)spiro[chromeno[4,3-d]thiazole-4,4′-piperidin]-2-yl)pyrimidine-5-carboxamide (5.0 mg, 0.010 mmol, 1.00 equiv.), (HCHO), (3.0 mg, 0.102 mmol, 10.00 equiv.), DCE (1.00 mL) and AcOH (1 drop) at room temperature. The resulting mixture was stirred for 0.5 h at room temperature. To the above mixture was added NaBH₃(CN) (2 mg, 0.030 mmol, 3.00 equiv.) at room temperature. The resulting mixture was stirred for additional 16 h at room temperature. The reaction was quenched with sat. NH₄Cl(aq.) at 0° C. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (DCM/MeOH (10:1)) to afford the title compound (3.5 mg, 68% yield) as a light yellow solid. ESI-MS m/z=522.21 [M+H]⁺; Calculated MW: 521.13; ¹H NMR (400 MHz, Chloroform-d) δ 10.90 (s, 1H), 8.38 (d, J=12.7 Hz, 1H), 7.66 (dd, J=28.7, 7.9 Hz, 1H), 7.22-7.15 (m, 2H), 4.03 (d, J=10.3 Hz, 6H), 3.63 (dt, J=36.2, 11.5 Hz, 1H), 3.48 (s, 1H), 2.76 (d, J=11.6 Hz, 2H), 2.57 (t, J=11.6 Hz, 1H), 2.39 (s, 2H), 2.34-2.16 (m, 3H), 2.12-1.97 (m, 1H).

### Example 47: N—(4,4-diethyl-7-(trifluoromethyl)-4H-chromeno[4,3-d]thiazol-2-yl)-4,6-diethoxypyrimidine-5-carboxamide

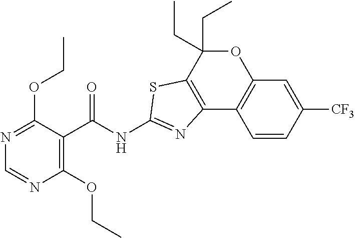 [SMILES: CCOc1ncnc(OCC)c1C(=O)Nc1nc2c(s1)C(CC)(CC)Oc1cc(C(F)(F)F)ccc1-2]

### Example 47A: 4,6-diethoxypyrimidine-5-carboxylic acid

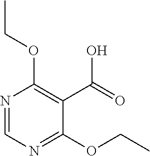

To a stirred solution EtOH (2.00 mL) was added KOH (117.0 mg, 2.08 mmol, 4 equiv.), 4,6-dichloropyrimidine-5-carboxylic acid (100.0 mg, 0.52 mmol, 1 equiv.) in portions at 0° C. The resulting mixture was stirred for 6 h at 80° C. The reaction was adjusted pH to 5 with HCl/dioxane (4M). The resulting mixture was filtered, the filter cake was washed with EtOH (3×20 mL). After filtration, the filtrate was concentrated under reduced pressure. This resulted in the title compound (120 mg, crude) as a light yellow solid. ESI-MS m/z=213.03 [M+H]⁺; Calculated MW: 212.08

### Example 47B: N—(4,4-diethyl-7-(trifluoromethyl)-4H-chromeno[4,3-d]thiazol-2-yl)-4,6-diethoxypyrimidine-5-carboxamide

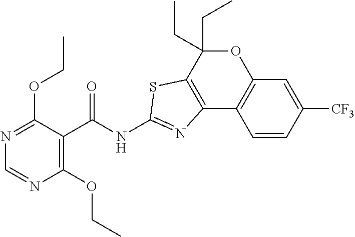 [SMILES: CCOc1ncnc(OCC)c1C(=O)Nc1nc2c(s1)C(CC)(CC)Oc1cc(C(F)(F)F)ccc1-2]

To a stirred solution of 4,6-diethoxypyrimidine-5-carboxylic acid (15 mg, 0.068 mmol, 1.50 equiv.) in DCM (1.0 mL) was added (COCl)₂ (29.0 mg, 0.228 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (15.00 mg, 0.046 mmol, 1.00 equiv.) in THF (1.0 mL) was added NaH (6.0 mg, 0.137 mmol, 3.00 equiv., 60%) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (3.3 mg, 13% yield) as a light yellow solid. ESI-MS m/z=523.12 [M+H]⁺; Calculated MW: 522.15; ¹H NMR (400 MHz, Chloroform-d) δ 10.77 (s, 1H), 8.33 (s, 1H), 7.61 (dt, J=7.5, 0.9 Hz, 1H), 7.13-7.07 (m, 2H), 4.51 (q, J=7.1 Hz, 4H), 2.02 (dq, J=14.7, 7.3 Hz, 2H), 1.89 (dt, J=14.7, 7.4 Hz, 2H), 1.42 (t, J=7.1 Hz, 6H), 0.98 (t, J=7.4 Hz, 6H).

### Example 48: N—(4,4-diethyl-7-(trifluoromethyl)-4H-chromeno[4,3-d]thiazol-2-yl)-4,6-bis(2,2,2-trifluoroethoxy)pyrimidine-5-carboxamide

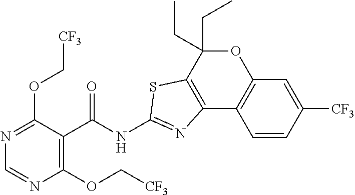 [SMILES: CCC1(CC)Oc2cc(C(F)(F)F)ccc2-c2nc(NC(=O)c3c(OCC(F)(F)F)ncnc3OCC(F)(F)F)sc21]

### Example 48A: 4,6-bis(2,2,2-trifluoroethoxy)pyrimidine-5-carboxylic acid

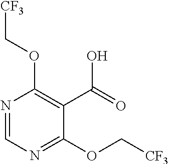 [SMILES: O=C(O)c1c(OCC(F)(F)F)ncnc1OCC(F)(F)F]

To a stirred solution CF 3CH₂OH (2.00 mL) was added KOH (117.0 mg, 2.08 mmol, 4 equiv.), 4,6-dichloropyrimidine-5-carboxylic acid (100.0 mg, 0.52 mmol, 1 equiv.) in portions at 0° C. The resulting mixture was stirred for 16 h at 75° C. The reaction was monitored by LCMS. Desired product could be detected by LCMS. The reaction was adjusted pH to 5 with HCl/dioxane (4M). The resulting mixture was filtered, the filter cake was washed with EtOH (3×20 mL). After filtration, the filtrate was concentrated under reduced pressure. This resulted in the title compound (110 mg, crude) as a light yellow solid. ESI-MS m/z=321.01 [M+H]⁺; Calculated MW: 320.02

### Example 48B: N—(4,4-diethyl-7-(trifluoromethyl)-4H-chromeno[4,3-d]thiazol-2-yl)-4,6-bis(2,2,2-trifluoroethoxy)pyrimidine-5-carboxamide

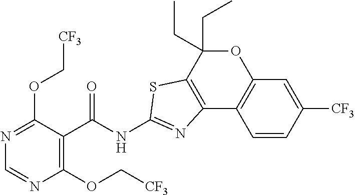 [SMILES: CCC1(CC)Oc2cc(C(F)(F)F)ccc2-c2nc(NC(=O)c3c(OCC(F)(F)F)ncnc3OCC(F)(F)F)sc21]

To a stirred solution of 4,6-bis(2,2,2-trifluoroethoxy)pyrimidine-5-carboxylic acid (15 mg, 0.047 mmol, 1.00 equiv) in DCM (1.00 mL) was added (COCl)₂ (30.00 mg, 0.234 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (15.00 mg, 0.047 mmol, 1.00 equiv.) in THF (1.00 mL) was added NaH (6.00 mg, 0.141 mmol, 3.00 equiv., 60%) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc/DCM (7:1:4)) to afford the title compound (10 mg, 34% yield) as a light yellow solid. ESI-MS m/z=631.00 [M+H]⁺; Calculated MW: 630.10; ¹H NMR (400 MHz, Chloroform-d) δ 10.30 (s, 1H), 8.41 (s, 1H), 7.60 (d, J=7.7 Hz, 1H), 7.12 (d, J=1.6 Hz, 1H), 7.09 (ddd, J=7.8, 1.8, 0.8 Hz, 1H), 4.88 (q, J=8.1 Hz, 4H), 2.08-1.98 (m, 2H), 1.91 (dq, J=14.6, 7.4 Hz, 2H), 1.00 (t, J=7.4 Hz, 6H).

### Example 49: N—(7-bromo-4,4-diethyl-4H-chromeno[4,3-d]thiazol-2-yl)-4,6-dimethoxypyrimidine-5-carboxamide

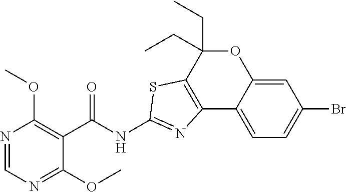 [SMILES: CCC1(CC)Oc2cc(Br)ccc2-c2nc(NC(=O)c3c(OC)ncnc3OC)sc21]

7-bromo-4,4-diethyl-4H-chromeno[4,3-d]thiazol-2-amine (1.0 g, 2.95 mmol, 1 equiv.) and 4,6-dimethoxypyrimidine-5-carboxylic acid (814.2 mg, 4.42 mmol, 1.5 equiv.) was added to a flame-dried three necked flask. The flask was evacuated and back-filled with Ar. ACN (10 mL) was added to the mixture and cooled to 0° C. NMI (847.0 mg, 0.817 mL, 10.32 mmol, 3.5 equiv.) was added to the mixture at 0° C. TCFH (1.24 g, 4.42 mmol, 1.5 equiv.) was dissolved in 10 mL ACN and added to the mixture at 0° C. The mixture was stirred at rt for 3 hours and stirred at 50° C. for 16 hours. The mixture was concentrated under reduce pressure then quenched with ice-water (20 mL). The aqueous was extracted with EA (20 mL×2). The combine organic layers were washed with brine (20 mL×2), dried over Na₂SO₄, filtrated and concentrated under reduce pressure. The crude product was purified by silica gel column chromatography, eluted with PE:EA:DCM=10:1:1 to afford the title compound (1.3 g, 87%) as yellow solid. ¹H NMR (400 MHz, Chloroform-d) δ 11.61 (s, 1H), 8.25 (s, 1H), 7.23 (d, J-8.1 Hz, 1H), 7.03 (d, J=1.8 Hz, 1H), 6.91 (dd, J=8.1, 1.9 Hz, 1H), 3.93 (s, 6H), 2.00-1.82 (m, 4H), 0.98 (t, J=7.4 Hz, 6H).

### Example 50: N—(7-cyano-4,4-diethyl-4H-chromeno[4,3-d]thiazol-2-yl)-4,6-dimethoxypyrimidine-5-carboxamide

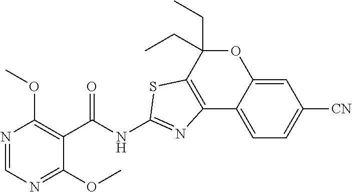 [SMILES: CCC1(CC)Oc2cc(C#N)ccc2-c2nc(NC(=O)c3c(OC)ncnc3OC)sc21]

N—(7-bromo-4,4-diethyl-4H-chromeno[4,3-d]thiazol-2-yl)-4,6-dimethoxypyrimidine-5-carboxamide (20 mg, 39.57 μmol, 1 equiv.), Zn(CN)₂ (13.94 mg, 118.72 μmol, 3 equiv.) and tBuXphos Pd G₃ (18.86 mg, 23.74 μmol, 0.6 equiv.) were added to a flame-dried tube. The tube was evacuated and back-filled with Ar. THF (0.2 mL) and H₂O (0.2 mL) were added to the mixture. The mixture was stirred at 60° C. for 16 hours, concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA=3:1) to afford the title compound (10 mg, 56%) as yellow solid. ESI-MS m/z=452.00 [M+H]⁺; Calculated MW: 451.50. ¹H NMR (400 MHz, Chloroform-d) δ 10.95 (s, 1H), 8.35 (d, J=1.6 Hz, 1H), 7.59-7.47 (m, 1H), 7.12 (dd, J=6.6, 1.7 Hz, 2H), 4.02 (d, J=1.5 Hz, 6H), 2.05-1.84 (m, 4H), 0.97 (td, J=7.4, 1.6 Hz, 6H).

### Example 51: N—(4,4-diethyl-4H-[1,3]dioxolo[4′,5′: 6,7]chromeno[4,3-d]thiazol-2-yl)-4,6-dimethoxypyrimidine-5-carboxamide

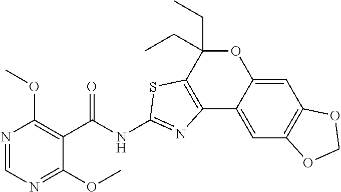 [SMILES: CCC1(CC)Oc2cc3c(cc2-c2nc(NC(=O)c4c(OC)ncnc4OC)sc21)OCO3]

### Example 51A: 1-(6-hydroxybenzo[d][1,3]dioxol-5-yl)ethan-1-one

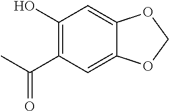

To a stirred solution of benzo[d][1,3]dioxol-5-ol (5.0 g, 36.2 mmol, 1.0 equiv.) and BF₃-Et₂O (mL9 mL, 144.8 mmol, 4.0 equiv.) in Ac₂O at 0° C. under nitrogen atmosphere. The reaction mixture was stirred for 1 hour at 90° C. Then the resulting mixture was added to the saturated potassium acetate solution and the resulting residue was extracted with ethyl acetate (3×20 mL). The combined organic layers were washed with brine, dried over anhydrous Na₂SO₄. The resulting mixture was concentrated under vacuum. The crude product was purified by silica gel column chromatography (PE:EA=4:1) to afford the title compound (4.5 g, 69%) as a yellow solid. ¹H NMR (400 MHz, Methanol-d₄) δ 7.22 (s, 1H), 6.39 (s, 1H), 5.97 (d, J=0.6 Hz, 2H), 2.50 (d, J=0.6 Hz, 3H).

### Example 51B: 6,6-diethyl-6,7-dihydro-8H-[1,3]dioxolo[4,5-g]chromen-8-one

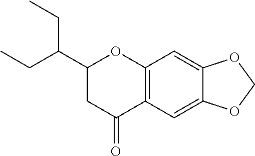 [SMILES: CCC(CC)C1CC(=O)c2cc3c(cc2O1)OCO3]

To a stirred solution of 1-(6-hydroxybenzo[d][1,3]dioxol-5-yl)ethan-1-one (3.5 g, 19.7 mmol, 1.0 equiv.) in EtOH (35 mL) was added pentan-3-one (4.2 g, 49.2 mmol, 2.5 equiv.) and pyrrolidine (3.5 g, 49.2 mmol, 2.5 equiv.) at 0° C. The reaction mixture was stirred overnight at 80° C. Then it was cooled to room temperature and ethanol was removed under vacuum. The resulting residue was extracted with ethyl acetate (3×35 mL). The combined organic layers were washed with brine and 10% NaOH aqueous solution, dried over anhydrous Na₂SO₄. The resulting mixture was concentrated under vacuum. The crude product was purified by silica gel column chromatography (PE:EA=10:1) to afford the title compound (1.1 g, 23%) as a yellow oil. ¹H NMR (400 MHz, Chloroform-d) δ 7.20 (s, 1H), 6.38 (s, 1H), 5.95 (s, 2H), 2.62 (s, 2H), 1.79 (dq, J=15.0, 7.5 Hz, 2H), 1.66 (dq, J=14.7, 7.4 Hz, 2H), 0.89 (t, J=7.5 Hz, 6H).

### Example 51C: 4,4-diethyl-4H-[1,3]dioxolo[4′,5′: 6,7]chromeno[4,3-d]thiazol-2-amine

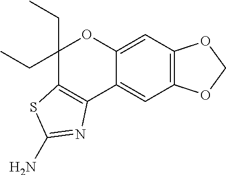 [SMILES: CCC1(CC)Oc2cc3c(cc2-c2nc(N)sc21)OCO3]

To a stirred solution of 6,6-diethyl-6,7-dihydro-8H-[1,3]dioxolo[4,5-g]chromen-8-one (1.1 g, 4.4 mmol, 1.0 equiv.) in toluene (10 mL) was added PTSA (165.3 mg, 0.9 mmol, 0.2 equiv.) and pyrrolidine (1.6 g, 22.2 mmol, 5.0 equiv.) at room temperature. The reaction mixture was stirred for 2 hours at 120° C. Then it was cooled to room temperature and toluene was removed under vacuum. The resulting mixture was dissolved in methanol (10 mL) and sulfur (354.2 mg, 11.07 mmol, 2.5 equiv) was added. The mixture was stirred for 10 minutes at room temperature and cyanamide (911.6 mg, 11.07 mmol, 2.5 equiv.) was added. The mixture was stirred overnight at room temperature. The resulting residue was extracted with ethyl acetate (mL3×10 mL). The combined organic layers were washed with brine, dried over anhydrous Na₂SO₄. The resulting mixture was concentrated under vacuum. The crude product was purified by silica gel column chromatography (PE:EA=4:1) to afford the title compound (10.0 mg, 1.0%) as a yellow oil. ¹H NMR (400 MHz, Chloroform-d) δ 7.03 (d, J=0.9 Hz, 1H), 6.43 (d, J=0.9 Hz, 1H), 5.88 (d, J=1.0 Hz, 2H), 5.29 (d, J=1.0 Hz, 2H), 1.88 (dq, J=14.7, 7.3 Hz, 2H), 1.74 (dq, J=14.6, 7.5 Hz, 2H), 0.94 (t, J=7.4 Hz, 6H).

### Example 51D: N—(4,4-diethyl-4H-[1,3]dioxolo[4′,5′: 6,7]chromeno[4,3-d]thiazol-2-yl)-4,6-dimethoxypyrimidine-5-carboxamide

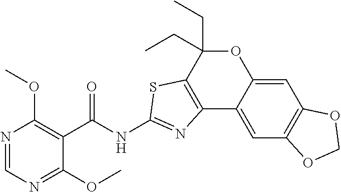 [SMILES: CCC1(CC)Oc2cc3c(cc2-c2nc(NC(=O)c4c(OC)ncnc4OC)sc21)OCO3]

To a suspension of 4,6-dimethoxypyrimidine-5-carboxylic acid (8.1 mg, 0.045 mmol, 1.5 equiv.) in dichloromethane (0.2 mL) and catalytic amount of DMF, was added oxalyl chloride (19.1 mg, 0.150 mmol, 5.0 equiv.) dropwise at 0° C., and the mixture was stirred for 2 hours at room temperature. After concentration, the mixture was dissolved in tetrahydrofuran (0.2 mL). 4,4-diethyl-4H-[1,3]dioxolo[4′,5′: 6,7]chromeno[4,3-d]thiazol-2-amine (19.1 mg, 0.150 mmol, 1.0 equiv.), KOH (5.0 mg, 0.090 mmol, 3.0 equiv.) were added at 0° C., and the mixture was stirred for 2 days at room temperature. After concentration, the resulting residue was extracted with ethyl acetate (3×5 mL). The combined organic layers were washed with brine, dried over anhydrous Na₂SO₄ and concentrated. The crude product was purified by Prep-TLC (PE:EA=1:1) to afford the title compound (9.4 mg, 2.0%) as a yellow solid. ESI-MS m/z=471.00 [M+H]⁺; Calculated MW: 470.13. ¹H NMR (400 MHz, Chloroform-d) & 11.62 (s, 1H), 8.24 (s, 1H), 6.84 (s, 1H), 6.45 (s, 1H), 5.90 (s, 2H), 3.90 (s, 6H), 1.96 (dt, J=14.4, 7.5 Hz, 2H), 1.86 (m, J=14.6, 7.4 Hz, 2H), 0.99 (t, J=7.4 Hz, 6H).

### Example 52: N—(4,4-diethyl-7-(trifluoromethyl)-4H-chromeno[4,3-d]thiazol-2-yl)-4,6-dimethylpyrimidine-5-carboxamide

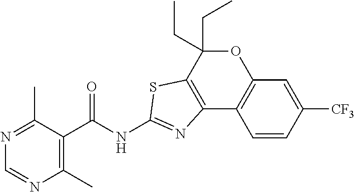 [SMILES: CCC1(CC)Oc2cc(C(F)(F)F)ccc2-c2nc(NC(=O)c3c(C)ncnc3C)sc21]

To a stirred solution of 4,6-dimethylpyrimidine-5-carboxylic acid (10.0 mg, 0.061 mmol, 1.00 equiv.), 4,4-diethyl-7-(trifluoromethyl)-4H-chromeno[4,3-d]thiazol-2-amine (20.0 mg, 0.061 mmol, 1.00 equiv.) in ACN (1.00 mL) was added NMI (7 mg, 0.213 mmol, 3.50 equiv.), TCFH (26 mg, 0.92 mmol, 1.50 equiv.) in portions at 0° C. The resulting mixture was stirred for 16 h at 60° C. The reaction was diluted with water, extracted with EtOAc (3×20 mL). The combined organic layers were washed with brine (2×20 mL), dried over anhydrous Na₂SO₄. After filtration, the filtrate was concentrated under reduced pressure. The residue was purified by prep-TLC (EA/PE=(1:1)) to afford the title compound (10 mg, 35% yield) as a light yellow solid. ESI-MS m/z=463.05 [M+H]⁺; Calculated MW: 462.13; 1H NMR (400 MHz, Chloroform-d) δ 10.42 (s, 1H), 8.87 (s, 1H), 7.48 (d, J=7.8 Hz, 1H), 7.15-7.07 (m, 2H), 2.47 (s, 6H), 2.05 (dq, J=14.8, 7.4 Hz, 2H), 1.93 (dq, J=14.6, 7.3 Hz, 2H), 1.02 (t, J=7.3 Hz, 6H).

### Example 53: N—(4,4-diethyl-7-(trifluoromethyl)-4H-chromeno[4,3-d]thiazol-2-yl)-4,6-dimethoxy-N-methylpyrimidine-5-carboxamide

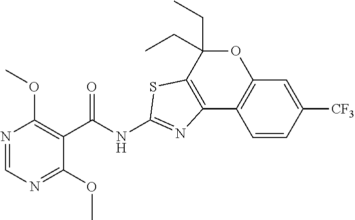 [SMILES: CCC1(CC)Oc2cc(C(F)(F)F)ccc2-c2nc(NC(=O)c3c(OC)ncnc3OC)sc21]

To a stirred solution of N—(4,4-diethyl-7-(trifluoromethyl)-4H-chromeno[4,3-d]thiazol-2-yl)-4,6-dimethoxy-N-methylpyrimidine-5-carboxamide (5.0 mg, 0.01 mmol, 1.00 equiv.) in DMF (0.50 mL) was added K₂CO₃ (3 mg, 0.02 mmol, 2.00 equiv.), Mel (14 mg, 0.10 mmol, 10.00 equiv.) in portions at 0° C. The resulting mixture was stirred for 16 h at room temperature. The reaction was diluted with water, then extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (EA/PE=(1:4)) to afford the title compound (3.5 mg, 68% yield) as an off-white solid. ESI-MS m/z=509.11 [M+H]⁺; Calculated MW: 508.14; ¹H NMR (400 MHz, Chloroform-d) δ 8.53 (d, J=2.7 Hz, 1H), 7.80 (d, J=7.9 Hz, 1H), 7.19-7.14 (m, 1H), 7.12 (s, 1H), 4.05-3.99 (m, 6H), 3.62 (d, J=2.7 Hz, 3H), 2.09-1.97 (m, 2H), 1.91 (ddd, J=14.4, 7.3, 2.5 Hz, 2H), 0.98 (td, J=7.4, 2.6 Hz, 6H).

### Example 54: N—(7-cyclopropyl-4,4-diethyl-4H-chromeno[4,3-d]thiazol-2-yl)-4,6-dimethoxypyrimidine-5-carboxamide

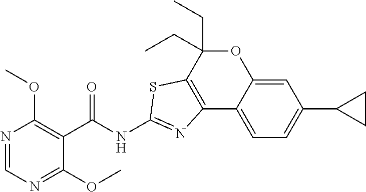 [SMILES: CCC1(CC)Oc2cc(C3CC3)ccc2-c2nc(NC(=O)c3c(OC)ncnc3OC)sc21]

N—(7-bromo-4,4-diethyl-4H-chromeno[4,3-d]thiazol-2-yl)-4,6-dimethoxypyrimidine-5-carboxamide (20 mg, 39.57 μmol, 1 equiv.), cyclopropyl boronic acid (10.2 mg, 118.72 μmol, 3 equiv.), K₃PO₄ (29.40 mg, 138.51 μmol, 3.5 equiv.), PCy₃ (3.33 mg, 11.87 μmol, 0.3 equiv.) and Pd(OAc)₂ (1.33 mg, 5.94 μmol, 0.15 equiv.) were added to a flame-dried tube. The tube was evacuated and back-filled with Ar. Toluene (0.2 mL) and H₂O (0.2 mL) were added to the mixture. The mixture was stirred at 100° C. for 16 hours. The mixture was concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA=3:1) to afford the title compound (10 mg, 54%) as yellow solid. ESI-MS m/z=467.08 [M+H]⁺; Calculated MW: 466.55. ¹H NMR (400 MHz, Chloroform-d) δ 11.75 (s, 1H), 8.19 (s, 1H), 7.22 (d, J=7.7 Hz, 1H), 6.55-6.47 (m, 2H), 3.90 (s, 6H), 1.91-1.80 (m, 4H), 0.99 (t, J=7.3 Hz, 6H).

### Example 55: N—(4,4-diethyl-7-(prop-1-en-2-yl)-4H-chromeno[4,3-d]thiazol-2-yl)-4,6-dimethoxypyrimidine-5-carboxamide

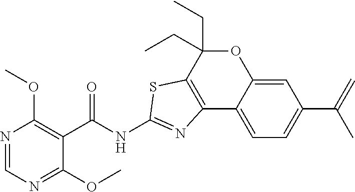 [SMILES: C=C(C)c1ccc2c(c1)OC(CC)(CC)c1sc(NC(=O)c3c(OC)ncnc3OC)nc1-2]

N—(7-bromo-4,4-diethyl-4H-chromeno[4,3-d]thiazol-2-yl)-4,6-dimethoxypyrimidine-5-carboxamide (100 mg, 197.81 μmol, 1 equiv.), 4,4,5,5-tetramethyl-2-(prop-1-en-2-yl)-1,3,2-dioxaborolane (66.50 mg, 395.74 μmol, 2 equiv.), CS₂CO₃ (193.41 mg, 593.60 μmol, 3.0 equiv.) and Pd(PPh₃)₄ (45.73 mg, 39.57 μmol, 0.2 equiv.) were added to a flame-dried tube. The tube was evacuated and back-filled with Ar. THF (2 mL) was added to the mixture. The mixture was stirred at 60° C. for 16 hours. The mixture was concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA=3:1) to afford the title compound (80 mg, 86.7%) as yellow solid. ESI-MS m/z=467.07 [M+H]⁺; Calculated MW: 466.55. ¹H NMR (400 MHz, Chloroform-d) δ 8.21 (s, 1H), 7.31 (d, J=8.0 Hz, 1H), 6.98 (s, 1H), 6.92 (d, J=7.9, 1H), 5.40 (s, 1H), 5.07 (s, 1H), 3.90 (s, 6H), 1.94 (ddt, J=35.6, 14.3, 7.2 Hz, 4H), 1.00 (t, J=7.4 Hz, 6H).

### Example 56: N—(4,4-diethyl-7-(trifluoromethyl)-4H-chromeno[4,3-d]thiazol-2-yl)-4,6-dimethoxypyrimidine-5-sulfonamide

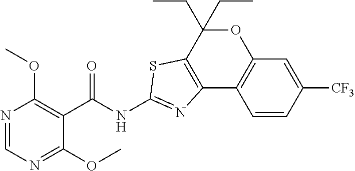 [SMILES: CCC1(CC)Oc2cc(C(F)(F)F)ccc2-c2nc(NC(=O)c3c(OC)ncnc3OC)sc21]

### Example 56A: 5-(benzylthio)-4,6-dimethoxypyrimidine

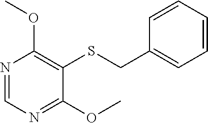 [SMILES: COc1ncnc(OC)c1SCc1ccccc1]

To a stirred solution of 5-bromo-4,6-dimethoxypyrimidine (200 mg, 0.913 mmol, 1.00 equiv.) in dioxane (2 mL) was added phenylmethanethiol (147 mg, 1.187 mmol, 1.3equiv.), DIEA (353 mg, 2.740 mmol, 3.0 equiv.), Xantphos (53 mg, 0.091 mmol, 0.1 equiv.) and Pd₂(dba)₃ (42 mg, 0.047 mmol, 0.05 equiv.) at room temperature under argon atmosphere. The resulting mixture was stirred for overnight at 110° C. Then the resulting mixture was cooled to room temperature and it was extracted with EA (2×15 mL). The combined organic layers were washed with brine (1×15 mL), dried over anhydrous Na₂SO₄. After filtration, the filtrate was concentrated under reduced pressure. The crude product was purified by prep-TLC (PE:EA=8:1) to afford the title compound (140 mg, 59%) as a yellow solid. ESI-MS m/z=262.92 [M+H]⁺; Calculated MW: 262.08.

### Example 56B: 4,6-dimethoxypyrimidine-5-sulfonyl chloride

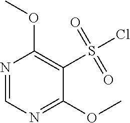

To a stirred solution of 5-(benzylthio)-4,6-dimethoxypyrimidine (140 mg, 0.534 mmol, 1.00 equiv.) in AcOH (1.5 mL) and H₂O (0.5 mL) was added NCS (286 mg, 2.137 mmol, 4.0 equiv.) at 0° C. Then the resulting mixture was stirred for overnight at room temperature under argon atmosphere. Then the resulting mixture was cooled to room temperature and it was extracted with EA (2×15 mL). The combined organic layers were washed with brine (1×15 mL), dried over anhydrous Na₂SO₄. After filtration, the filtrate was concentrated under reduced pressure. The crude product was purified by prep-TLC (PE:EA=7:1) to afford the title compound (50 mg, 39%) as a yellow solid. ESI-MS m/z=238.83 [M+H]⁺; Calculated MW: 237.98.

### Example 56C: N—(4,4-diethyl-7-(trifluoromethyl)-4H-chromeno[4,3-d]thiazol-2-yl)-4,6-dimethoxypyrimidine-5-sulfonamide

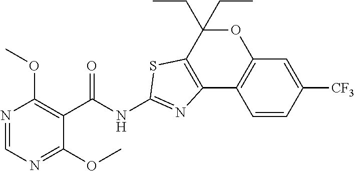 [SMILES: CCC1(CC)Oc2cc(C(F)(F)F)ccc2-c2nc(NC(=O)c3c(OC)ncnc3OC)sc21]

To a suspension of 4,6-dimethoxypyrimidine-5-sulfonyl chloride (20 mg, 0.084 mmol, 1.20 equiv.) in pyridine (0.4 mL) was added 4,4-diethyl-7-(trifluoromethyl)-4H-chromeno[4,3-d]thiazol-2-amine (23 mg, 0.070 mmol, 1.0 equiv.) at 0° C. Then the mixture was stirred overnight at room temperature. Then the resulting mixture was extracted with EA (2×10 mL) and the combined organic layers were washed with brine (1×10 mL), dried over anhydrous Na₂SO₄. After filtration, the filtrate was concentrated under reduced pressure. The crude product was purified by prep-TLC (PE:EA=2:1) to afford the title compound (2.8 mg, 7.6%) as a white solid. ESI-MS m/z=530.95 [M+H]⁺; Calculated MW: 530.09. ¹H NMR (400 MHz, Chloroform-d) δ 8.40 (s, 1H), 7.38 (d, J=8.0 Hz, 1H), 7.20-7.16 (m, 1H), 7.15-7.11 (m, 1H), 4.00 (s, 6H), 1.98 (dq, J=14.7, 7.3 Hz, 2H), 1.82 (dq, J=14.7, 7.4 Hz, 2H), 0.97 (t, J=7.3 Hz, 6H).

### Example 57: N—(4,4-diethyl-7-(trifluoromethyl)-4H-chromeno[4,3-d]thiazol-2-yl)-4-methoxy-6-methylpyrimidine-5-carboxamide

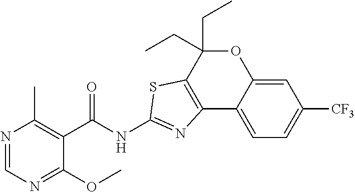 [SMILES: CCC1(CC)Oc2cc(C(F)(F)F)ccc2-c2nc(NC(=O)c3c(C)ncnc3OC)sc21]

### Example 57A: 4-methoxy-6-methylpyrimidine-5-carboxylic acid

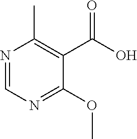

To a stirred solution of ethyl 4-chloro-6-methylpyrimidine-5-carboxylate (50 mg, 0.249 mmol, 1.00 equiv.) in MeOH (1 mL) was added KOH (21 mg, 0.373 mmol, 1.50 equiv.) at 0° C. The mixture was stirred overnight at 70° C. Then it was cooled to 0° C. and adjusted to pH to 5-6 with 4 N HCl/dioxane. The mixture was filtered through a Celite pad and washed with MeOH (2×5 mL). The filtrate was concentrated under vacuum to afford the title compound as yellow oil (50 mg, crude). ESI-MS m/z=168.92 [M+H]⁺; Calculated MW: 168.05.

### Example 57B: N—(4,4-diethyl-7-(trifluoromethyl)-4H-chromeno[4,3-d]thiazol-2-yl)-4-methoxy-6-methylpyrimidine-5-carboxamide

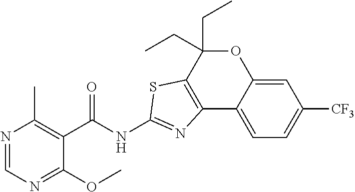 [SMILES: CCC1(CC)Oc2cc(C(F)(F)F)ccc2-c2nc(NC(=O)c3c(C)ncnc3OC)sc21]

To a suspension of 4-methoxy-6-methylpyrimidine-5-carboxylic acid (30 mg, 0.178 mmol, 1.20 equiv.) in CH₂Cl₂ (0.5 mL) and catalytic amount of DMF, oxalyl chloride (47 mg, 0.372 mmol, 2.5 equiv.) was added dropwise at 0° C., and the mixture was stirred for 3 h at room temperature. Then the reaction mixture was concentrated in vacuo and dissolved in THF (0.5 mL). 4,4-diethyl-7-(trifluoromethyl)-4H-chromeno[4,3-d]thiazol-2-amine (49 mg, 0.149 mmol, 1.0 equiv.) and KOH (0.65 mg, 0.446 mmol, 3.0 equiv.) were added, and the mixture was stirred overnight at room temperature. Then the resulting mixture was extracted with EA (2×10 mL) and the combined organic layers were washed with brine (1×10 mL), dried over anhydrous Na₂SO₄. After filtration, the filtrate was concentrated under reduced pressure. The crude product was purified by prep-TLC (DCM:PE:EA=7:4:1) to afford the title compound (3.5 mg, 4.9%) as yellow oil. ESI-MS m/z=479.06 [M+H]⁺; Calculated MW: 478.13. ¹H NMR (400 MHz, Chloroform-d) δ 10.83 (s, 1H), 8.63 (s, 1H), 7.62-7.55 (m, 1H), 7.16-7.08 (m, 2H), 4.05 (s, 3H), 2.70 (s, 3H), 2.03 (dq, J=14.7, 7.4 Hz, 2H), 1.91 (dq, J=14.7, 7.4 Hz, 2H), 1.00 (t, J=7.4 Hz, 6H).

### Example 58: N—(4,4-diethyl-7-morpholino-4H-chromeno[4,3-d]thiazol-2-yl)-4,6-dimethoxypyrimidine-5-carboxamide

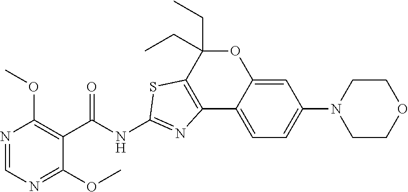 [SMILES: CCC1(CC)Oc2cc(N3CCOCC3)ccc2-c2nc(NC(=O)c3c(OC)ncnc3OC)sc21]

N—(7-bromo-4,4-diethyl-4H-chromeno[4,3-d]thiazol-2-yl)-4,6-dimethoxypyrimidine-5-carboxamide (20 mg, 39.57 μmol, 1 equiv.), morpholine (10.34 mg, 118.72 μmol, 3 equiv.), CS₂CO₃ (38.68 mg, 118.72 μmol, 3.0 equiv.) and Ruphos Pd G₂ (6.5 mg, 7.91 μmol, 0.2 equiv.) were added to a flame-dried tube. The tube was evacuated and back-filled with Ar. 1,4-dioxane (0.2 mL) was added to the mixture. The mixture was stirred at 100° C. for 16 hours. The mixture was concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA=3:1) to afford the title compound (10 mg, 49%) as yellow solid. ESI-MS m/z=512.13 [M+H]⁺; Calculated MW: 511.59. ¹H NMR (400 MHz, Chloroform-d) δ 11.36 (s, 1H), 8.28 (s, 1H), 7.30 (d, J=8.5 Hz, 1H), 6.47-6.31 (m, 2H), 3.95 (s, 6H), 3.90-3.80 (m, 4H), 3.17-3.07 (m, 4H), 1.93 (dtt, J=34.4, 14.3, 6.6 Hz, 4H), 0.98 (t, J=7.4 Hz, 6H).

### Example 59: 4,6-dimethoxy-N—(7-(trifluoromethyl)-2′,3′,5′,6′-tetrahydrospiro[chromeno[4,3-d]thiazole-4,4′-pyran]-2-yl)pyrimidine-5-carboxamide

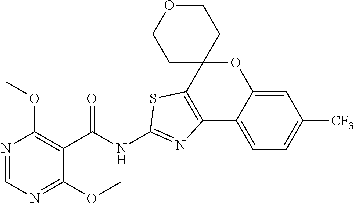 [SMILES: COc1ncnc(OC)c1C(=O)Nc1nc2c(s1)C1(CCOCC1)Oc1cc(C(F)(F)F)ccc1-2]

### Example 59A: 7-(trifluoromethyl)-2′,3′,5′,6′-tetrahydrospiro[chromane-2,4′-pyran]-4-one

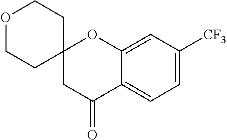 [SMILES: O=C1CC2(CCOCC2)Oc2cc(C(F)(F)F)ccc21]

To a stirred solution of 1-(2-hydroxy-4-(trifluoromethyl)phenyl)ethan-1-one (100 mg, 0.489 mmol, 1.00 equiv.) and tetrahydro-4H-pyran-4-one (49 mg, 0.489 mmol, 1.00 equiv.) in MeOH (1 mL) was added pyrrolidine (70 mg, 0.979 mmol, 2.00 equiv.) at 0° C. The resulting mixture was stirred for 5 h at room temperature. The mixture was concentrated under reduced pressure. The crude product was purified by silica gel column chromatography (PE:EA=4:1) to afford the title compound (71 mg, 51%) as a yellow solid. ¹H NMR (400 MHz, Chloroform-d) δ 7.95 (dt, J=8.2, 0.8 Hz, 1H), 7.29 (d, J=1.6 Hz, 1H), 7.23 (ddd, J=8.2, 1.6, 0.7 Hz, 1H), 3.88-3.73 (m, 4H), 2.78 (s, 2H), 1.97 (dq, J=14.4, 2.7 Hz, 2H), 1.79 (ddd, J=13.9, 10.9, 5.2 Hz, 2H).

### Example 59B: 7-(trifluoromethyl)-2′,3′,5′,6′-tetrahydrospiro[chromeno[4,3-d]thiazole-4,4′-pyran]-2-amine

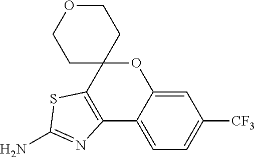 [SMILES: Nc1nc2c(s1)C1(CCOCC1)Oc1cc(C(F)(F)F)ccc1-2]

To a suspension of 7-(trifluoromethyl)-2′,3′,5′,6′-tetrahydrospiro[chromane-2,4′-pyran]-4-one (100 mg, 0.349 mmol, 1.00 equiv.) and TsOH-H₂O (6.6 mg, 0.0349 mmol, 0.10 equiv.) in n-hexane (2.5 mL), pyrrolidine (124.2 mg, 1.75 mmol, 5.00 equiv.) was added at room temperature, and the mixture was stirred for 2 h at 80° C. Then the reaction mixture was concentrated in vacuo and dissolved in MeOH (1 mL). S (28 mg, 0.873 mmol, 2.5 equiv.) was added at 0° C. and the mixture was stirred for 5 mins at 0° C. Then 51% w/w % cyanamide (72.00 mg, 0.873 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=3:1) to afford the title compound (13 mg, 11%) as a yellow solid. ¹H NMR (400 MHz, Chloroform-d) δ 7.66 (dd, J=7.9, 1.0 Hz, 1H), 7.24-7.17 (m, 2H), 5.01 (s, 2H), 3.94 (td, J=11.7, 2.2 Hz, 2H), 3.88-3.78 (m, 2H), 2.17-2.09 (m, 2H), 1.93 (ddd, J=13.8, 11.8, 5.2 Hz, 2H).

### Example 59C: 4,6-dimethoxy-N—(7-(trifluoromethyl)-2′,3′,5′,6′-tetrahydrospiro[chromeno[4,3-d]thiazole-4,4′-pyran]-2-yl)pyrimidine-5-carboxamide

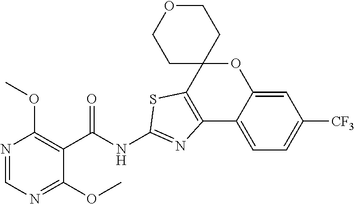 [SMILES: COc1ncnc(OC)c1C(=O)Nc1nc2c(s1)C1(CCOCC1)Oc1cc(C(F)(F)F)ccc1-2]

To a suspension of 7-(trifluoromethyl)-2′,3′,5′,6′-tetrahydrospiro[chromeno[4,3-d]thiazole-4,4′-pyran]-2-amine (13 mg, 0.038 mmol, 1.00 equiv.) and 4,6-dimethoxypyrimidine-5-carboxylic acid (10.5 mg, 0.057 mmol, 1.50 equiv.) in ACN (0.5 mL). NMI (10.91 mg, 0.133 mmol, 3.50 equiv.) and TCFH (16 mg, 0.057 mmol, 1.50 equiv.) were added dropwise at 0° C., and the mixture was stirred for 16 h at 50° C. Then the reaction mixture was concentrated in vacuo. The resulting mixture was extracted with EA (2×10 mL) and washed with H₂O (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=2:1) to afford the title compound (3.2 mg, 17%) as a white solid. ESI-MS m/z=508.99 [M+H]⁺; Calculated MW: 508.47. ¹H NMR (400 MHz, Chloroform-d) δ 10.52 (s, 1H), 8.43 (s, 1H), 7.69 (d, J=7.8 Hz, 1H), 7.22 (dd, J=9.7, 1.4 Hz, 2H), 4.08 (s, 6H), 4.02-3.85 (m, 4H), 2.27-2.10 (m, 2H), 2.10-1.97 (m, 2H).

### Example 60: N—(4,4-diethyl-7-(trifluoromethyl)-4H-thiazolo[4′,5′: 4,5]pyrano[2,3-b]pyridin-2-yl)-4,6-dimethoxypyrimidine-5-carboxamide

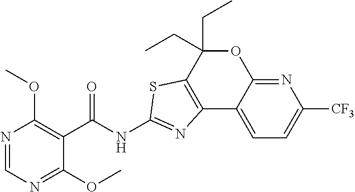 [SMILES: CCC1(CC)Oc2nc(C(F)(F)F)ccc2-c2nc(NC(=O)c3c(OC)ncnc3OC)sc21]

### Example 60A: 3-acetyl-6-(trifluoromethyl)pyridin-2 (1H)-one

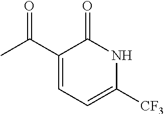

To a stirred solution of (E)-4-ethoxy-1,1,1-trifluorobut-3-en-2-one (2 g, 11.9 mmol, 1.00 equiv.) and 3-oxobutanamide (1.2 g, 11.9 mmol, 1.00 equiv.) in EtOH (20 mL) was added sodium ethoxide (4.4 mL, 11.9 mmol, 1.00 equiv.) at 0° C., and the mixture was stirred for 12 h at 75° C. The mixture was quenched with 1M hydrochloric acid, then the resulting mixture was extracted with EA (2×10 mL) and washed with sat. aq. NaHCO₃The combined organic layers were washed with brine (1×10 mL), dried over anhydrous Na₂SO₄. After filtration, the filtrate was concentrated under reduced pressure. The crude product was purified by silica gel chromatography (PE:EA=3:1) to afford the title compound (1.73 g, 71%) as a yellow solid. ¹H NMR (400 MHz, Chloroform-d) δ 8.30 (d, J=7.9 Hz, 1H), 7.34 (d, J=7.9 Hz, 1H), 2.72 (s, 3H).

### Example 60B: 2,2-diethyl-7-(trifluoromethyl)-2,3-dihydro-4H-pyrano[2,3-b]pyridin-4-one

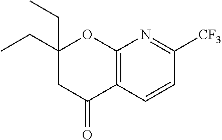 [SMILES: CCC1(CC)CC(=O)c2ccc(C(F)(F)F)nc2O1]

To a suspension of 3-acetyl-6-(trifluoromethyl)pyridin-2 (1H)-one (1 g, 4.87 mmol, 1.00 equiv.) in 3-Pentanone (10 mL) were added 3A Molecular Sieves (0.625 g) and pyrrolidine (346.1 mg, 4.87 mmol, 1.00 equiv.) at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=5:1) to afford the title compound (400 mg, 30%) as a yellow solid. ¹H NMR (400 MHz, DMSO-d₆) δ 8.43-8.22 (m, 1H), 7.56 (dd, J=7.7, 1.1 Hz, 1H), 2.92 (s, 2H), 1.77-1.64 (m, J=7.4 Hz, 4H), 0.85 (t, J=7.4 Hz, 6H).

### Example 60C: 4,4-diethyl-7-(trifluoromethyl)-4H-thiazolo[4′,5′: 4,5]pyrano[2,3-b]pyridin-2-amine

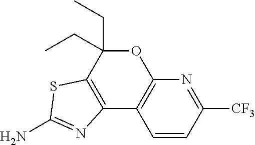 [SMILES: CCC1(CC)Oc2nc(C(F)(F)F)ccc2-c2nc(N)sc21]

To a suspension of 2,2-diethyl-7-(trifluoromethyl)-2,3-dihydro-4H-pyrano[2,3-b]pyridin-4-one (100 mg, 0.360 mmol, 1.00 equiv.) and TsOH-H₂O (6.8 mg, 0.036 mmol, 0.10 equiv.) in Toluene (4 mL), pyrrolidine (128 mg, 1.8 mmol, 5.00 equiv.) was added at room temperature, and the mixture was stirred for 2 h at 120° C. Then the reaction mixture was concentrated in vacuo and dissolved in MeOH (2 mL). S (28.8 mg, 0.9 mmol, 2.5 equiv.) was added at 0° C. and the mixture was stirred for 5 min at 0° C. Then 51% w/w % cyanamide (73.4 mg, 0.9 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=10:1) to afford the title compound (15 mg, 13%) as a yellow solid. ESI-MS m/z=329.92 [M+H]⁺; Calculated MW: 329.08.

### Example 60D: N—(4,4-diethyl-7-(trifluoromethyl)-4H-thiazolo[4′,5′: 4,5]pyrano[2,3-b]pyridin-2-yl)-4,6-dimethoxypyrimidine-5-carboxamide

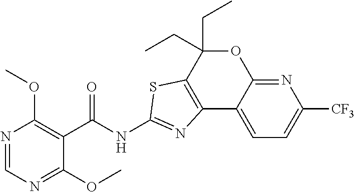 [SMILES: CCC1(CC)Oc2nc(C(F)(F)F)ccc2-c2nc(NC(=O)c3c(OC)ncnc3OC)sc21]

To a suspension of 4,6-dimethoxypyrimidine-5-carboxylic acid (11.4 mg, 0.073 mmol, 1.20 equiv.) in CH₂Cl₂ (0.5 mL) and catalytic amount of DMF, oxalyl chloride (19.3 mg, 0.152 mmol, 2.50 equiv.) was added dropwise at 0° C., and the mixture was stirred for 2 h at room temperature. Then the reaction mixture was concentrated in vacuo and dissolved in THF (0.5 mL). 4,4-diethyl-7-(trifluoromethyl)-4H-thiazolo[4′,5′: 4,5]pyrano[2,3-b]pyridin-2-amine (20 mg, 0.0608 mmol, 1.0 equiv.), KOH (10.2 mg, 0.1824 mmol, 3.0 equiv.) and THF (0.5 mL) were added and the mixture was stirred overnight at room temperature. Then the resulting mixture was extracted with EA (2×10 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by TLC (PE:EA=1:1) to afford the title compound (1.9 mg, 6.3%) as a white solid. ESI-MS m/z=496.01 [M+H]⁺; Calculated MW: 495.12. ¹H NMR (400 MHz, Chloroform-d) δ 10.34 (s, 1H), 8.48 (s, 1H), 7.96 (dd, J=7.5, 0.8 Hz, 1H), 4.12 (s, 6H), 2.16 (dp, J=14.6, 7.6 Hz, 2H), 1.93 (dq, J=14.7, 7.4 Hz, 2H), 0.97 (t, J=7.4 Hz, 6H).

### Example 61: N—(4,4-diethyl-7-(2-hydroxypropan-2-yl)-4H-chromeno[4,3-d]thiazol-2-yl)-4,6-dimethoxypyrimidine-5-carboxamide

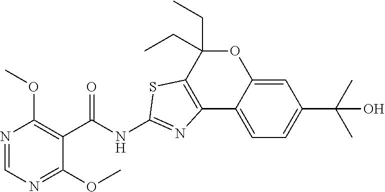 [SMILES: CCC1(CC)Oc2cc(C(C)(C)O)ccc2-c2nc(NC(=O)c3c(OC)ncnc3OC)sc21]

To a flame-dried Schlenk tube equipped with a magnetic stir bar was charged with N—(4,4-diethyl-7-(prop-1-en-2-yl)-4H-chromeno[4,3-d]thiazol-2-yl)-4,6-dimethoxypyrimidine-5-carboxamide (20 mg, 42.87 μmol, 1.0 equiv.), methyl 4-nitrobenzenesulfonate (12.1 mg, 55.73 μmol, 1.30 equiv.), NaHCO₃ (7.2 mg, 85.73 μmol, 2.0 equiv.) and Fe(acac)₃ (1.15 mg, 4.29 μmol, 0.1 equiv.). The reaction vessel was evacuated and backfilled with argon and then dry MeOH (0.4 mL) was added under argon atmosphere. The resultant mixture was cooled to 0° C. followed by addition of PhSiH₃ (13.9 mg, 128.60 umol, 3.0 equiv.). The mixture was stirred at rt for 16 hours. The mixture was concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA=1:1) to afford the title compound (5 mg, 24%) as yellow solid. ESI-MS m/z=485.05 [M+H]⁺; Calculated MW: 484.57. ¹H NMR (400 MHz, Chloroform-d) δ 11.68 (s, 1H), 8.18 (s, 1H), 7.31 (d, J=7.9 Hz, 1H), 7.04-6.87 (m, 2H), 3.92 (s, 6H), 1.94 (ddt, J=35.6, 14.3, 7.2 Hz, 4H), 1.00 (t, J=7.3 Hz, 6H).

### Example 62: ethyl 2-(4,6-dimethoxypyrimidine-5-carboxamido)-4,4-diethyl-4H-chromeno[4,3-d]thiazole-7-carboxylate

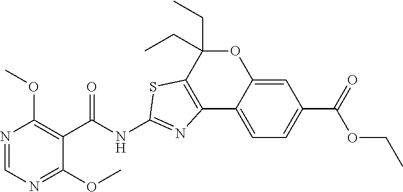 [SMILES: CCOC(=O)c1ccc2c(c1)OC(CC)(CC)c1sc(NC(=O)c3c(OC)ncnc3OC)nc1-2]

### Example 62A

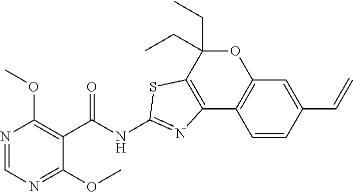 [SMILES: C=Cc1ccc2c(c1)OC(CC)(CC)c1sc(NC(=O)c3c(OC)ncnc3OC)nc1-2]

N—(7-bromo-4,4-diethyl-4H-chromeno[4,3-d]thiazol-2-yl)-4,6-dimethoxypyrimidine-5-carboxamide (20 mg, 39.57 μmol, 1 equiv.), 4,4,5,5-tetramethyl-2-vinyl-1,3,2-dioxaborolane (12.2 mg, 79.15 μmol, 2 equiv.), K₃PO₄ (25.2 mg, 118.72 μmol, 3.0 equiv.), Sphos (4.9 mg, 11.87 μmol, 0.3 equiv.) and Pd(OAc)₂ (1.3 mg, 5.94 μmol, 0.15 equiv.) were added to a flame-dried tube. The tube was evacuated and back-filled with Ar. 1,4-dioxane (0.3 mL) and H₂O (20 μl) were added to the mixture. The mixture was stirred at 100° C. for 16 hours. The mixture was concentrated under reduce pressure then quenched with ice-water (5 mL). The aqueous was extracted with EA (10 mL×2). The combine organic layers were washed with brine (20 mL×2), dried over Na₂SO₄, filtrated, and concentrated under reduce pressure. The crude product was purified by prep-TLC (PE:EA=3:1) to afford the title compound (10 mg, 56%) as yellow solid. ESI-MS m/z=453.05 [M+H]⁺; Calculated MW: 452.52. ¹H NMR (400 MHz, Chloroform-d) δ 12.47-12.19 (m, 1H), 8.10 (d, J=3.0 Hz, 1H), 7.22 (dd, J=7.8, 2.4 Hz, 1H), 6.91 (d, J=1.6 Hz, 1H), 6.77 (dd, J=7.8, 1.7 Hz, 1H), 6.62 (dd, J=17.5, 10.9 Hz, 1H), 5.72 (d, J=17.5 Hz, 1H), 5.23 (d, J=10.9 Hz, 1H), 3.81 (s, 6H), 1.94 (dp, J=34.9, 7.3 Hz, 4H), 1.01 (t, J=7.4 Hz, 6H).

### Example 62B: N—(4,4-diethyl-7-formyl-4H-chromeno[4,3-d]thiazol-2-yl)-4,6-dimethoxypyrimidine-5-carboxamide

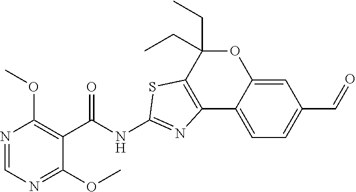 [SMILES: CCC1(CC)Oc2cc(C=O)ccc2-c2nc(NC(=O)c3c(OC)ncnc3OC)sc21]

To a flame-dried Schlenk tube equipped with a magnetic stir bar was charged with N—(4,4-diethyl-7-vinyl-4H-chromeno[4,3-d]thiazol-2-yl)-4,6-dimethoxypyrimidine-5-carboxamide (127 mg, 280.642 μmol, 1.0 equiv.), ACN (1 mL), Acetone (1 mL), H₂O (1 mL) and DCM (1 mL). The resultant mixture was cooled to 0° C. followed by addition of KIO₄ (258.2 mg, 1.12 mmol, 4 equiv.) and K₂OsO₄ (9.3 mg, 28.06 μmol, 0.1 equiv.). The mixture was stirred at rt for 3 hours. The mixture was filtrated and concentrated under reduce pressure then quenched with ice-water (5 mL). The aqueous was extracted with EA (10 mL×2). The combine organic layers were washed with brine (20 mL×2), dried over Na₂SO₄, filtered and concentrated under reduce pressure. The crude product was purified by prep-TLC (PE:EA=3:1) to afford the title compound (20 mg, 16%) as yellow solid. ¹H NMR (400 MHz, Chloroform-d) δ 11.30 (s, 1H), 9.89 (s, 1H), 8.27 (s, 1H), 7.58 (d, J=8.1 Hz, 1H), 7.41-7.32 (m, 2H), 3.97 (s, 6H), 2.03-1.96 (m, 2H), 1.92 (dt, J=14.3, 7.4 Hz, 2H), 1.00 (t, J=7.4 Hz, 6H).

### Example 62C: 2-(4,6-dimethoxypyrimidine-5-carboxamido)-4,4-diethyl-4H-chromeno[4,3-d]thiazole-7-carboxylic acid

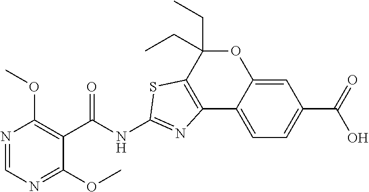 [SMILES: CCC1(CC)Oc2cc(C(=O)O)ccc2-c2nc(NC(=O)c3c(OC)ncnc3OC)sc21]

To a flame-dried Schlenk tube equipped with a magnetic stir bar was charged with N—(4,4-diethyl-7-formyl-4H-chromeno[4,3-d]thiazol-2-yl)-4,6-dimethoxypyrimidine-5-carboxamide (20 mg, 44.0 μmol, 1.0 equiv.), THF (0.2 mL) and H₂O (0.2 mL). The resultant mixture was cooled to 0° C. followed by addition of KMnO₄ (10.4 mg, 66.01 μmol, 1.5 equiv.). The mixture was stirred at rt for 1 hours. The mixture was filtrated and concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA=3:1) to afford the title compound (10 mg, 48%) as yellow solid. ESI-MS m/z=471.97 [M+H]⁺; Calculated MW: 470.50.

### Example 62D: ethyl 2-(4,6-dimethoxypyrimidine-5-carboxamido)-4,4-diethyl-4H-chromeno[4,3-d]thiazole-7-carboxylate

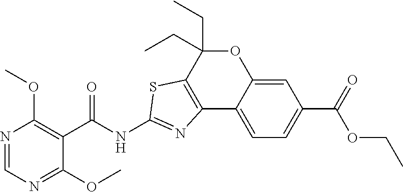 [SMILES: CCOC(=O)c1ccc2c(c1)OC(CC)(CC)c1sc(NC(=O)c3c(OC)ncnc3OC)nc1-2]

To a flame-dried Schlenk tube equipped with a magnetic stir bar was charged with 2-(4,6-dimethoxypyrimidine-5-carboxamido)-4,4-diethyl-4H-chromeno[4,3-d]thiazole-7-carboxylic acid (10 mg, 21.52 μmol, 1.0 equiv.), H₂SO₄ (20 μl) and EtOH (0.2 mL). The mixture was stirred at 68° C. for 16 hours. The mixture was quenched with ice-water (5 mL). The aqueous was basified to PH=8 with NaHCO₃ aqueous then extracted with EA (10 mL×2). The combine organic layers were washed with brine (20 mL×2), dried over Na₂SO₄, filtrated and concentrated under reduce pressure. The crude product was purified by prep-TLC (PE:EA=3:1) to afford the title compound (2 mg, 19%) as yellow solid. ESI-MS m/z=499.02 [M+H]⁺; Calculated MW: 498.55. ¹H NMR (400 MHz, Chloroform-d) δ 10.45 (s, 1H), 8.43 (d, J=4.6 Hz, 1H), 7.68-7.50 (m, 3H), 4.34 (q, J=6.5 Hz, 2H), 4.09 (d, J=4.6 Hz, 6H), 2.00 (d, J=7.9 Hz, 2H), 1.93-1.86 (m, 2H), 1.40-1.36 (m, 3H), 0.97 (q, J=6.7 Hz, 6H).

### Example 63: tert-butyl 2′-(4,6-dimethoxypyrimidine-5-carboxamido)-7′-(trifluoromethyl)spiro[azetidine-3,4′-chromeno[4,3-d]thiazole]-1-carboxylate

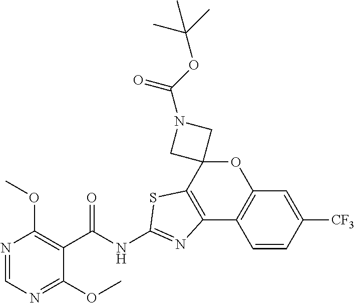 [SMILES: COc1ncnc(OC)c1C(=O)Nc1nc2c(s1)C1(CN(C(=O)OC(C)(C)C)C1)Oc1cc(C(F)(F)F)ccc1-2]

### Example 63A: tert-butyl 4′-oxo-7′-(trifluoromethyl)spiro[azetidine-3,2′-chromane]-1-carboxylate

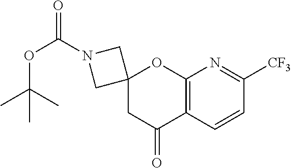 [SMILES: CC(C)(C)OC(=O)N1CC2(CC(=O)c3ccc(C(F)(F)F)nc3O2)C1]

To a stirred solution of 1-(2-hydroxy-4-(trifluoromethyl)phenyl)ethan-1-one (500.0 mg, 2.45 mmol, 1.00 equiv.) and tert-butyl 3-oxoazetidine-1-carboxylate (544.0 mg, 3.18 mmol, 1.30 equiv.) in MeOH (5.0 mL) was added pyrrolidine (349.0 mg, 4.90 mmol, 2.00 equiv.) in portions at 0° C. The resulting mixture was stirred for 16 h at 60° C. under argon atmosphere. The reaction was diluted with water (100 mL), extracted with EtOAc (3×50 mL). The combined organic layers were washed with brine (2×30 mL), dried over anhydrous Na₂SO₄. After filtration, the filtrate was concentrated under reduced pressure. The residue was purified by prep-TLC (PE/EtOAc (10:1)) to afford the title compound (550 mg, 62% yield) as a light yellow solid. ¹H NMR (400 MHz, Chloroform-d) δ 7.97 (d, J=8.2 Hz, 1H), 7.34 (d, J=1.6 Hz, 1H), 7.32-7.27 (m, 1H), 4.12-4.05 (m, 2H), 3.97 (dd, J=9.6, 1.1 Hz, 2H), 3.08 (s, 2H), 1.43 (d, J=1.2 Hz, 9H).

### Example 63B: tert-butyl 2′-amino-7′-(trifluoromethyl)spiro[azetidine-3,4′-chromeno[4,3-d]thiazole]-1-carboxylate

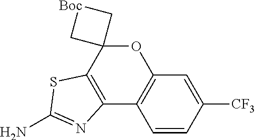

Into a 50 mL round-bottom flask were added tert-butyl 4′-oxo-7′-(trifluoromethyl)spiro[azetidine-3,2′-chromane]-1-carboxylate (550 mg, 1.540 mmol, 1.00 equiv.), hexane (15 mL), molecular sieve, pyrrolidine (547 mg, 7.70 mmol, 5.00 equiv.), and TsOH·H₂O (1.5 mg, 0.015 mmol, 0.10 equiv.) at room temperature. The resulting mixture was stirred for 3 h at 80° C. under argon atmosphere. The resulting mixture was cooled down to room temperature, then concentrated under reduced pressure. To the above mixture was added MeOH (6 mL), S (123 mg, 3.85 mmol, 2.50 equiv.) and NH₂CN (317 mg, 3.85 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×50 mL). The combined organic layers were washed with brine (2×20 mL), dried over anhydrous Na₂SO₄. After filtration, the filtrate was concentrated under reduced pressure. The residue was purified by prep-TLC (PE/EtOAc (2:1)) to afford the title compound (100 mg, 16% yield) as a light yellow oil. ESI-MS m/z=414.06 [M+H]⁺; Calculated MW: 413.10

### Example 63C: tert-butyl 2′-(4,6-dimethoxypyrimidine-5-carboxamido)-7′-(trifluoromethyl)spiro[azetidine-3,4′-chromeno[4,3-d]thiazole]-1-carboxylate

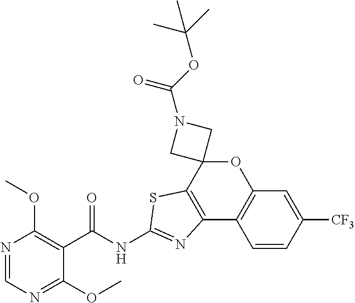 [SMILES: COc1ncnc(OC)c1C(=O)Nc1nc2c(s1)C1(CN(C(=O)OC(C)(C)C)C1)Oc1cc(C(F)(F)F)ccc1-2]

To a stirred solution of 4,6-dimethoxypyrimidine-5-carboxylic acid (30.0 mg, 0.073 mmol, 1.50 equiv.) in DCM (1.00 mL) was added (COCl)₂ (50.0 mg, 0.363 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of tert-butyl 2′-amino-7′-(trifluoromethyl)spiro[azetidine-3,4′-chromeno[4,3-d]thiazole]-1-carboxylate (20.0 mg, 0.073 mmol, 1.00 equiv.) in THF (1.00 mL) was added KOH (13.0 mg, 0.218 mmol, 3.00 equiv.) in portions at 0° C. The resulting mixture was stirred for 0.5 h at 0° C. under argon atmosphere. To the above mixture was added the acyl chloride/THE dropwise at 0° C. The resulting mixture was stirred for additional 16 h at 0° C. to room temperature. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (20 mg, 47% yield) as a light yellow solid. ESI-MS m/z=579.99 [M+H]⁺; Calculated MW: 579.14; 1H NMR (400 MHz, Chloroform-d) δ 11.32 (d, J=25.6 Hz, 1H), 8.34-8.19 (m, 1H), 7.55 (dd, J=8.0, 4.5 Hz, 1H), 7.22 (d, J=1.6 Hz, 1H), 7.18-7.13 (m, 1H), 4.44 (dd, J=9.8, 1.0 Hz, 2H), 4.29-4.22 (m, 2H), 4.01-3.90 (m, 6H), 1.48 (s, 9H).

### Example 64: N—(1′-acetyl-7-(trifluoromethyl)spiro[chromeno[4,3-d]thiazole-4,4′-piperidin]-2-yl)-4,6-dimethoxypyrimidine-5-carboxamide

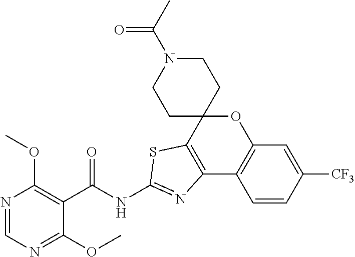 [SMILES: COc1ncnc(OC)c1C(=O)Nc1nc2c(s1)C1(CCN(C(C)=O)CC1)Oc1cc(C(F)(F)F)ccc1-2]

To a stirred solution of 4,6-dimethoxy-N—(7-(trifluoromethyl)spiro[chromeno[4,3-d]thiazole-4,4′-piperidin]-2-yl)pyrimidine-5-carboxamide (5.0 mg, 0.010 mmol, 1.00 equiv.) in DCM (1.00 mL) was added TEA (10 mg, 0.100 mmol, 10.00 equiv.), acetyl chloride (0.8 mg, 0.010 mmol, 1.00 equiv.) in DCM (0.2 mL) in portions at 0° C. The resulting mixture was stirred for 0.5 h at 0° C. The reaction was diluted with DCM at 0° C., then washed with water (2×10 mL), dried over anhydrous Na₂SO₄. After filtration, the filtrate was concentrated under reduced pressure. The residue was purified by prep-TLC (EA/PE=(2:1)) to afford the title compound (2 mg, 36% yield) as a white solid. ESI-MS m/z=550.03 [M+H]⁺; Calculated MW: 549.13; ¹H NMR (400 MHz, Chloroform-d) δ 10.64 (s, 1H), 8.40 (s, 1H), 7.72-7.65 (m, 1H), 7.22 (d, J=7.1 Hz, 2H), 4.56 (d, J=13.6 Hz, 1H), 4.05 (s, 6H), 3.80-3.59 (m, 2H), 3.15 (t, J=12.3 Hz, 1H), 2.35-2.23 (m, 2H), 2.16 (s, 2H), 2.03 (s, 1H), 1.88 (qd, J=13.7, 4.9 Hz, 2H).

### Example 65: 4,6-dimethoxy-N—(7-(trifluoromethyl)spiro[chromeno[4,3-d]thiazole-4,3′-oxetan]-2-yl)pyrimidine-5-carboxamide

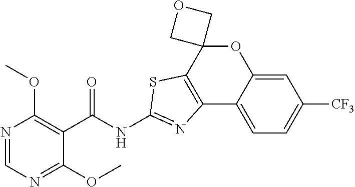 [SMILES: COc1ncnc(OC)c1C(=O)Nc1nc2c(s1)C1(COC1)Oc1cc(C(F)(F)F)ccc1-2]

### Example 65A: 7-(trifluoromethyl)spiro[chromane-2,3′-oxetan]-4-one

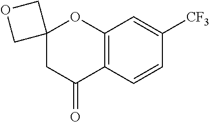 [SMILES: O=C1CC2(COC2)Oc2cc(C(F)(F)F)ccc21]

To a stirred solution of 1-(2-hydroxy-4-(trifluoromethyl)phenyl)ethan-1-one (300 mg, 1.47 mmol, 1.0equiv.) in methanol (3 mL) was added oxetan-3-one (0.17 mL, 2.94 mmol, 2.0equiv.) and pyrrolidine (0.25 mL, 2.94 mmol, 2.0equiv.) at 0° C. The reaction mixture was stirred overnight at room temperature and concentrated. The resulting residue was extracted with ethyl acetate (3×5 mL). The combined organic layers were washed with brine, dried over anhydrous Na₂SO₄. The resulting mixture was concentrated under vacuum. The crude product was purified by silica gel column chromatography (PE:EA=4:1) to afford the title compound (350 mg, 93%) as a yellow solid. ¹H NMR (400 MHz, Chloroform-d) δ 7.96 (d, J=8.2 Hz, 1H), 7.37 (d, J=1.6 Hz, 1H), 7.28 (dd, J=8.2, 1.6 Hz, 1H), 4.82 (d, J=7.4 Hz, 2H), 4.66-4.55 (m, 2H), 3.20 (s, 2H).

### Example 65B: 7-(trifluoromethyl)spiro[chromeno[4,3-d]thiazole-4,3′-oxetan]-2-amine

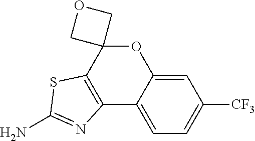 [SMILES: Nc1nc2c(s1)C1(COC1)Oc1cc(C(F)(F)F)ccc1-2]

To a stirred solution of 7-(trifluoromethyl)spiro[chromane-2,3′-oxetan]-4-one (400 mg, 1.55 mmol, 1.0equiv.) in toluene (4 mL) was added PTSA (158.9 mg, 0.31 mmol, 0.2equiv.) and pyrrolidine (550.3 mg, 7.75 mmol, 5.0equiv.) at room temperature. The reaction mixture was stirred for 2 hours at 120° C. Then it was cooled to room temperature and concentrated. Then the resulting mixture was dissolved in methanol (10 mL) and sulfur (124.2 mg, 3.88 mmol, 2.5equiv.) was added. The mixture was stirred for 10 minutes at room temperature. Cyanamide was added (162.9 mg, 3.88 mmol, 2.5equiv.) and the mixture was stirred overnight at room temperature. After concentration the resulting residue was extracted with ethyl acetate (3×4 mL). The combined organic layers were washed with brine, dried over anhydrous Na₂SO₄. The resulting mixture was concentrated under vacuum. The crude product was purified by silica gel column chromatography (PE:EA=1:1) to afford the title compound (330 mg, 68%) as a yellow solid. ¹H NMR (400 MHz, DMSO-d₆) δ 7.57 (s, 1H), 7.32-7.26 (m, 2H), 4.89 (d, J=8.0 Hz, 2H), 4.78-4.73 (m, 2H).

### Example 65C: 4,6-dimethoxy-N—(7-(trifluoromethyl)spiro[chromeno[4,3-d]thiazole-4,3′-oxetan]-2-yl)pyrimidine-5-carboxamide

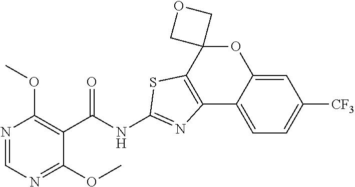 [SMILES: COc1ncnc(OC)c1C(=O)Nc1nc2c(s1)C1(COC1)Oc1cc(C(F)(F)F)ccc1-2]

To a stirred solution of 7-(trifluoromethyl)spiro[chromeno[4,3-d]thiazole-4,3′-oxetan]-2-amine (150 mg, 0.48 mmol, 1.0 equiv.) in acetonitrile (1.5 mL) was added 4,6-dimethoxypyrimidine-5-carboxylic acid (86.4 mg, 0.48 mmol, 1.0equiv.), TCFH (201.6 mg, 0.72 mmol, 1.5equiv.) and NMI (129.6 mg, 1.58 mmol, 3.5equiv.) at room temperature. The reaction mixture was stirred overnight at 50° C. After concentration, the resulting residue was extracted with ethyl acetate (3×5 mL). The combined organic layers were washed with brine, dried over anhydrous Na₂SO₄. The resulting mixture was concentrated under vacuum. The crude product was purified by Prep-TLC (PE:EA=4:1) to afford the title compound (4.3 mg, 6.2%) as a yellow solid. ESI-MS m/z=480.96 [M+H]⁺; Calculated MW: 480.07. ¹H NMR (400 MHz, Chloroform-d) δ 10.51 (s, 1H), 8.45 (s, 1H), 7.70 (d, J=7.9 Hz, 1H), 7.27 (d, J=1.6 Hz, 1H), 7.22 (d, J=1.1 Hz, 1H), 5.21-5.05 (m, 2H), 4.99-4.85 (m, 2H), 4.10 (s, 6H).

### Example 66: N—(4,4-bis(methoxymethyl)-7-(trifluoromethyl)-4H-chromeno[4,3-d]thiazol-2-yl)-4,6-dimethoxypyrimidine-5-carboxamide

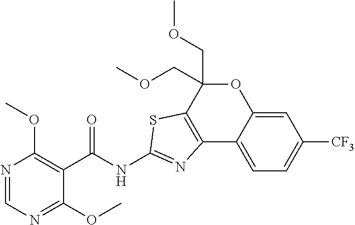 [SMILES: COCC1(COC)Oc2cc(C(F)(F)F)ccc2-c2nc(NC(=O)c3c(OC)ncnc3OC)sc21]

### Example 66A: 2,2-bis(methoxymethyl)-7-(trifluoromethyl) chroman-4-one

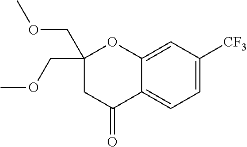 [SMILES: COCC1(COC)CC(=O)c2ccc(C(F)(F)F)cc2O1]

To a stirred solution of 1-(2-hydroxy-4-(trifluoromethyl)phenyl)ethan-1-one (100 mg, 0.489 mmol, 1.00 equiv.) and 1,3-dimethoxypropan-2-one (57.8 mg, 0.489 mmol, 1.00 equiv.) in MeOH (1 mL) was added pyrrolidine (87.1 mg, 1.22 mmol, 2.50 equiv.) at 0° C. The resulting mixture was stirred for 5 h at room temperature. The mixture was concentrated under reduced pressure. The crude product was purified by silica gel column chromatography (PE:EA=3.5:1) to afford the title compound (96 mg, 64%) as a yellow solid. ¹H NMR (400 MHz, Chloroform-d) δ 7.92 (d, J=8.1 Hz, 1H), 7.29-7.24 (m, 1H), 7.20 (ddd, J=8.1, 1.6, 0.7 Hz, 1H), 3.61-3.50 (m, 4H), 3.35 (s, 6H), 2.94 (s, 2H).

### Example 66B: 4,4-bis(methoxymethyl)-7-(trifluoromethyl)-4H-chromeno[4,3-d]thiazol-2-amine

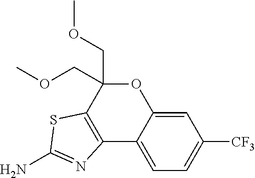 [SMILES: COCC1(COC)Oc2cc(C(F)(F)F)ccc2-c2nc(N)sc21]

To a suspension of 2,2-bis(methoxymethyl)-7-(trifluoromethyl) chroman-4-one (96 mg, 0.315 mmol, 1.00 equiv.) and TsOH-H₂O (6.0 mg, 0.0315 mmol, 0.10 equiv.) in n-hexane (2.5 mL) was added pyrrolidine (112.2 mg, 1.58 mmol, 5.00 equiv.) at room temperature, and the mixture was stirred for 2 h at 80° C. Then the reaction mixture was concentrated in vacuo and dissolved in MeOH (1 mL), S (25.3 mg, 0.788 mmol, 2.5 equiv.) was added at 0° C. and the mixture was stirred for 5 mins at 0° C. Then 51% w/w % cyanamide (65.00 mg, 0.788 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=1:1) to afford the title compound (18.7 mg, 16%) as a yellow solid. ¹H NMR (400 MHz, Chloroform-d) δ 7.66 (d, J=7.9 Hz, 1H), 7.21 (d, J=7.9 Hz, 1H), 7.16 (d, J=1.9 Hz, 1H), 5.13 (s, 2H), 3.76 (d, J=9.9 Hz, 2H), 3.60 (d, J=10.0 Hz, 2H), 3.40 (s, 6H).

### Example 66C: N—(4,4-bis(methoxymethyl)-7-(trifluoromethyl)-4H-chromeno[4,3-d]thiazol-2-yl)-4,6-dimethoxypyrimidine-5-carboxamide

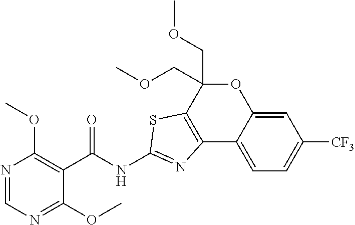 [SMILES: COCC1(COC)Oc2cc(C(F)(F)F)ccc2-c2nc(NC(=O)c3c(OC)ncnc3OC)sc21]

To a suspension of 4,4-bis(methoxymethyl)-7-(trifluoromethyl)-4H-chromeno[4,3-d]thiazol-2-amine (18.7 mg, 0.052 mmol, 1.00 equiv.) and 4,6-dimethoxypyrimidine-5-carboxylic acid (14.3 mg, 0.0778 mmol, 1.50 equiv.) in ACN (0.5 mL).NMI (14.9 mg, 0.181 mmol, 3.50 equiv.) and TCFH (21.8 mg, 0.078 mmol, 1.50 equiv.) were added dropwise at 0° C. The mixture was stirred for 16 h at 50° C. Then the reaction mixture was concentrated in vacuo. The resulting mixture was extracted with EA (2×10 mL) and washed with H₂O (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=2:1) to afford the title compound (5.2 mg, 19%) as a white solid. ESI-MS m/z=527.07 [M+H]⁺; Calculated MW: 526.49. ¹H NMR (400 MHz, Chloroform-d) δ 10.80 (s, 1H), 8.37 (s, 1H), 7.64 (d, J=7.8 Hz, 1H), 7.21-7.13 (m, 2H), 4.03 (s, 6H), 3.81 (d, J=9.9 Hz, 2H), 3.68 (d, J=9.9 Hz, 2H), 3.43 (s, 6H).

### Example 67: 4,6-dimethoxy-N—(7′-(trifluoromethyl)spiro[azetidine-3,4′-chromeno[4,3-d]thiazol]-2′-yl)pyrimidine-5-carboxamide

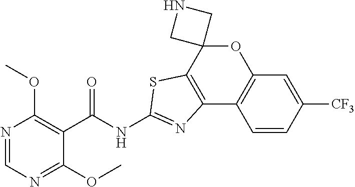 [SMILES: COc1ncnc(OC)c1C(=O)Nc1nc2c(s1)C1(CNC1)Oc1cc(C(F)(F)F)ccc1-2]

To a stirred solution of tert-butyl 2′-(4,6-dimethoxypyrimidine-5-carboxamido)-7′-(trifluoromethyl)spiro[azetidine-3,4′-chromeno[4,3-d]thiazole]-1-carboxylate (18.0 mg, 0.031 mmol, 1.00 equiv.) in DCM (1.5 mL) was added TFA (0.5 mL) in portions at 0° C. The resulting mixture was stirred for 1 h at room temperature. The reaction was concentrated under reduced pressure. The residue was diluted with water, adjusted pH to 9 with NaHCO₃ (aq.). The resulting mixture was extracted with EtOAc (3×30 mL). The combined organic layers were washed with brine (2×10 mL), dried over anhydrous Na₂SO₄. After filtration, the filtrate was concentrated under reduced pressure. This resulted in the title compound (10.0 mg, 68% yield) as an off-white solid. ESI-MS m/z=479.95 [M+H]⁺; Calculated MW: 479.09; ¹H NMR (400 MHz, Chloroform-d) δ 11.50 (s, 1H), 8.23 (s, 1H), 7.50 (d, J=7.9 Hz, 1H), 7.19 (d, J=1.7 Hz, 1H), 7.10 (dd, J=8.1, 1.7 Hz, 1H), 4.22-4.15 (m, 2H), 3.94-3.88 (m, 8H).

### Example 68: N—(1-acetyl-7′-(trifluoromethyl)spiro[azetidine-3,4′-chromeno[4,3-d]thiazol]-2′-yl)-4,6-dimethoxypyrimidine-5-carboxamide

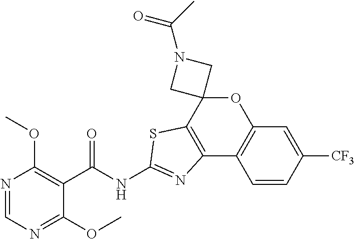 [SMILES: COc1ncnc(OC)c1C(=O)Nc1nc2c(s1)C1(CN(C(C)=O)C1)Oc1cc(C(F)(F)F)ccc1-2]

To a stirred solution of 4,6-dimethoxy-N—(7′-(trifluoromethyl)spiro[azetidine-3,4′-chromeno[4,3-d]thiazol]-2′-yl)pyrimidine-5-carboxamide (10.0 mg, 0.021 mmol, 1.00 equiv.) in DCM (1.00 mL) was added TEA (21 mg, 0.210 mmol, 10.00 equiv.), acetyl chloride (1.6 mg, 0.021 mmol, 1.00 equiv.) in DCM (0.2 mL) in portions at 0° C. The resulting mixture was stirred for 0.5 h at 0° C. The reaction was diluted with DCM at 0° C., then washed with water (2×10 mL), dried over anhydrous Na₂SO₄. After filtration, the filtrate was concentrated under reduced pressure. The residue was purified by prep-TLC (EA/PE=(2:1)) to afford the title compound (5 mg, 45% yield) as a white solid. ESI-MS m/z=522.01 [M+H]⁺; Calculated MW: 521.10; ¹H NMR (400 MHz, Chloroform-d) δ 10.68 (s, 1H), 8.43 (s, 1H), 7.70 (dt, J=7.8, 0.8 Hz, 1H), 7.26 (dd, J=1.8, 0.8 Hz, 1H), 7.24 (td, J=1.7, 0.8 Hz, 2H), 4.63 (dd, J=9.5, 1.3 Hz, 1H), 4.51 (d, J=11.0 Hz, 1H), 4.44 (dd, J=9.5, 1.3 Hz, 1H), 4.38-4.32 (m, 1H), 4.08 (s, 6H), 1.97 (s, 3H).

### Example 69: N—(1′-(cyclopropanecarbonyl)-7-(trifluoromethyl)spiro[chromeno[4,3-d]thiazole-4,4′-piperidin]-2-yl)-4,6-dimethoxypyrimidine-5-carboxamide

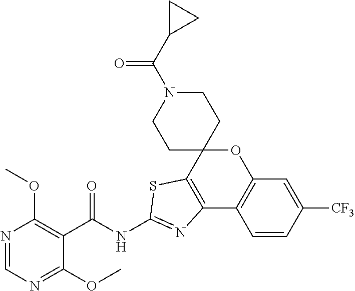 [SMILES: COc1ncnc(OC)c1C(=O)Nc1nc2c(s1)C1(CCN(C(=O)C3CC3)CC1)Oc1cc(C(F)(F)F)ccc1-2]

To a stirred solution of 4,6-dimethoxy-N—(7-(trifluoromethyl)spiro[chromeno[4,3-d]thiazole-4,4′-piperidin]-2-yl)pyrimidine-5-carboxamide (4.0 mg, 0.008 mmol, 1.00 equiv.) in DCM (1.00 mL) was added TEA (8 mg, 0.079 mmol, 10.00 equiv.) and cyclopropanecarbonyl chloride (0.8 mg, 0.008 mmol, 1.00 equiv.) in DCM (0.2 mL) in portions at 0° C. The resulting mixture was stirred for 1 h at 0° C. The reaction was diluted with DCM at 0° C., then washed with water (2×10 mL), dried over anhydrous Na₂SO₄. After filtration, the filtrate was concentrated under reduced pressure. The residue was purified by prep-TLC (EA/PE=(2:1)) to afford the title compound (2 mg, 44% yield) as a white solid. ESI-MS m/z=576.03 [M+H]⁺; Calculated MW: 575.15; ¹H NMR (400 MHz, Chloroform-d) δ 10.89 (s, 1H), 8.36 (s, 1H), 7.64 (d, J=7.6 Hz, 1H), 7.23-7.17 (m, 2H), 4.55 (d, J=13.4 Hz, 1H), 4.21-4.12 (m, 1H), 4.01 (s, 6H), 3.71 (t, J=13.0 Hz, 1H), 3.19 (t, J=12.9 Hz, 1H), 2.40-2.24 (m, 2H), 2.23-2.18 (m, 1H), 2.03-1.85 (m, 1H), 1.79 (tt, J=8.0, 4.7 Hz, 1H), 1.02 (dt, J=6.0, 3.0 Hz, 2H), 0.90-0.84 (m, 2H).

### Example 70: N—(4,4-diethyl-7-(trifluoromethyl)-4H-chromeno[4,3-d]thiazol-2-yl)-4-methoxy-6-(2-methoxyethoxy)pyrimidine-5-carboxamide

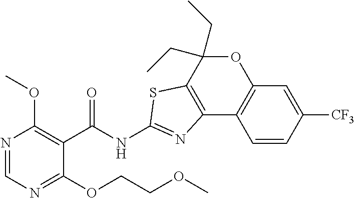 [SMILES: CCC1(CC)Oc2cc(C(F)(F)F)ccc2-c2nc(NC(=O)c3c(OC)ncnc3OCCOC)sc21]

### Example 70A: 4-methoxy-6-(2-methoxyethoxy)pyrimidine-5-carboxylic acid

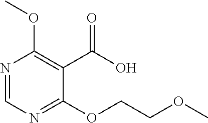 [SMILES: COCCOc1ncnc(OC)c1C(=O)O]

To a stirred solution of 2-methoxyethan-1-ol (79.0 mg, 1.042 mmol, 1.00 equiv.) in THF (2.00 mL) was added NaH (96.0 mg, 2.29 mmol, 2.20 equiv., 60%) in portions at 0° C. The resulting mixture was stirred for 0.5 h at 0° C. under argon. To the above mixture was added 4,6-dichloropyrimidine-5-carboxylic acid (200 mg, 1.042 mmol, 1.00 equiv.) in THF (1 mL) dropwise at 0° C. The resulting mixture was stirred for additional 16 h at 0° C. to room temperature. Upon the formation of the desired intermediate, to the above mixture was added MeONa/MeOH (0.25 mL, 1.25 mmol, 1.20 equiv.) dropwise at 0° C. The resulting mixture was stirred for additional 3 h at 0° C. to room temperature. The reaction was diluted with water, adjusted pH to 5 with HCl (1M), extracted with EtOAc (3×50 mL). The combined organic layers were washed with brine (2×20 mL), dried over anhydrous Na₂SO₄. After filtration, the filtrate was concentrated under reduced pressure. This resulted in the title compound (230 mg, crude) as a light yellow solid. ESI-MS m/z=228.91 [M+H]⁺; Calculated MW: 228.07.

### Example 70B: N—(4,4-diethyl-7-(trifluoromethyl)-4H-chromeno[4,3-d]thiazol-2-yl)-4-methoxy-6-(2-methoxyethoxy)pyrimidine-5-carboxamide

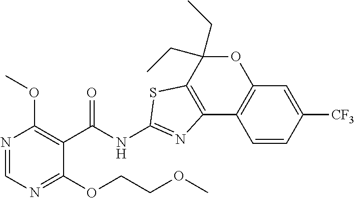 [SMILES: CCC1(CC)Oc2cc(C(F)(F)F)ccc2-c2nc(NC(=O)c3c(OC)ncnc3OCCOC)sc21]

To a stirred solution of 4-methoxy-6-(2-methoxyethoxy)pyrimidine-5-carboxylic acid (20 mg, 0.228 mmol, 1.50 equiv.) in DCM (1.00 mL) was added (COCl)₂ (97.0 mg, 0.762 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (15.0 mg, 0.047 mmol, 1.00 equiv.) in THF (1.00 mL) was added KOH (26.0 mg, 0.457 mmol, 3.00 equiv.) in portions at 0° C. The resulting mixture was stirred for 0.5 h at 0° C. under argon atmosphere. To the above mixture was added the acyl chloride/THE dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (15.0 mg, 12.2% yield) as a light yellow solid. ESI-MS m/z=539.04 [M+H]⁺; Calculated MW: 538.15; ¹H NMR (400 MHz, Chloroform-d) δ 10.95 (s, 1H), 8.39 (s, 1H), 7.60 (dd, J=8.2, 1.1 Hz, 1H), 7.14-7.07 (m, 2H), 4.67-4.62 (m, 2H), 4.05 (s, 3H), 3.80-3.75 (m, 2H), 3.54 (s, 3H), 2.00 (dt, J=14.7, 7.3 Hz, 2H), 1.89 (dq, J=14.6, 7.4 Hz, 2H), 0.97 (t, J=7.4 Hz, 6H).

### Example 71: N—(7-cyanonaphtho[1,2-d]thiazol-2-yl)-4,6-dimethoxypyrimidine-5-carboxamide

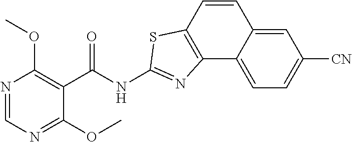 [SMILES: COc1ncnc(OC)c1C(=O)Nc1nc2c(ccc3cc(C#N)ccc32)s1]

### Example 71A: 2-aminonaphtho[1,2-d]thiazole-7-carbonitrile

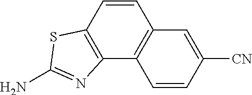 [SMILES: N#Cc1ccc2c(ccc3sc(N)nc32)c1]

To a stirred solution of 5-oxo-5,6,7,8-tetrahydronaphthalene-2-carbonitrile (100 mg, 0.59 mmol, 1.0equiv.) in DMSO (2 mL) was added PTSA (560.5 mg, 2.95 mmol, 5.0 equiv.) and iodine (45.7 mg, 0.18 mmol, 0.3 equiv.) at room temperature under oxygen atmosphere. The reaction mixture was stirred overnight at room temperature. The resulting residue was extracted with ethyl acetate (3×4 mL). The combined organic layers were washed with brine, dried over anhydrous Na₂SO₄. The resulting mixture was concentrated under vacuum. The crude product was purified by Prep-TLC (PE:EA=1:1) to afford the title compound (20 mg, 15%) as a yellow solid. ESI-MS m/z=225.88 [M+H]⁺; Calculated MW: 225.04

### Example 71B: N—(7-cyanonaphtho[1,2-d]thiazol-2-yl)-4,6-dimethoxypyrimidine-5-carboxamide

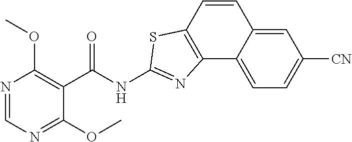 [SMILES: COc1ncnc(OC)c1C(=O)Nc1nc2c(ccc3cc(C#N)ccc32)s1]

To a suspension of 4,6-dimethoxypyrimidine-5-carboxylic acid (24.1 mg, 0.134 mmol, 1.5equiv.) in dichloromethane (0.2 mL) and catalytic amount of DMF, was added oxalyl chloride (56.5 mg, 0.445 mmol, 5.0equiv.) dropwise at 0° C., and the mixture was stirred for 2 hours at room temperature. After concentration, the mixture was dissolved in tetrahydrofuran (0.5 mL), 2-aminonaphtho[1,2-d]thiazole-7-carbonitrile (20.0 mg, 0.089 mmol, 1.0equiv.), KOH (14.9 mg, 0.267 mmol, 3.0 equiv.) were added at 0° C., and the mixture was stirred for 2 days at room temperature. After concentration, the resulting residue was extracted with ethyl acetate (3×5 mL). The combined organic layers were washed with brine, dried over anhydrous Na₂SO₄. The resulting mixture was concentrated under vacuum. The crude product was purified by Prep-TLC (DCM:EA=10:1) to afford the title compound (1.0 mg, 2.0%) as a white solid. ESI-MS m/z=391.95 [M+H]⁺; Calculated MW: 391.07. ¹H NMR (400 MHz, Chloroform-d) δ 10.42 (s, 1H), 8.71 (d, J=8.5 Hz, 1H), 8.51 (s, 1H), 8.32 (d, J=1.6 Hz, 1H), 8.00 (d, J=8.7 Hz, 1H), 7.82-7.74 (m, 2H), 4.17 (s, 6H).

### Example 72: 4,6-dimethoxy-N—(7-(trifluoromethyl) thiazolo[5,4-c]quinolin-2-yl)pyrimidine-5-carboxamide

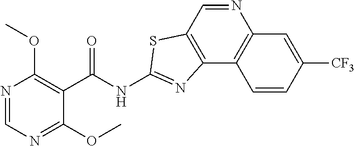 [SMILES: COc1ncnc(OC)c1C(=O)Nc1nc2c(cnc3cc(C(F)(F)F)ccc32)s1]

### Example 72A: 3-((3-(trifluoromethyl)phenyl)amino)propanoic acid

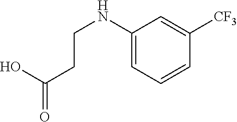 [SMILES: O=C(O)CCNc1cccc(C(F)(F)F)c1]

To a stirred solution of 3-(trifluoromethyl) aniline hydrochloride (1.00 g, 5.00 mmol, 1.00 equiv.) in H₂O (5.0 mL) was added sodium acrylate (716.0 mg, 7.5 mmol, 1.5 equiv.) in portions at 0° C. The resulting mixture was stirred for 3 h at 100° C. under argon. The reaction was cooled down to room temperature, adjusted pH to 5 with HCl (1M), extracted with EtOAc (3×50 mL). The combined organic layers were washed with brine (2×20 mL), dried over anhydrous Na₂SO₄. After filtration, the filtrate was concentrated under reduced pressure. This resulted in the title compound (980 mg, crude) as a light yellow oil. ESI-MS m/z=233.88 [M+H]⁺; Calculated MW: 233.07

### Example 72B: 7-(trifluoromethyl)-2,3-dihydroquinolin-4 (1H)-one

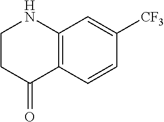 [SMILES: O=C1CCNc2cc(C(F)(F)F)ccc21]

To a stirred solution of polyphosphoric acid (5 mL) was added 3-((3-(trifluoromethyl)phenyl)amino)propanoic acid (980 mg, 4.21 mmol, 1.00 equiv.) in portions at room temperature. The resulting mixture was stirred for 1 h at 120° C. under argon atmosphere. The reaction was cooled down to room temperature, then poured it into ice water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EtOAc (4:1) to afford the title compound (95 mg, 11% yield) as a yellow solid. ESI-MS m/z=215.90 [M+H]⁺; Calculated MW: 215.06

### Example 72C: 7-(trifluoromethyl) thiazolo[5,4-c]quinolin-2-amine

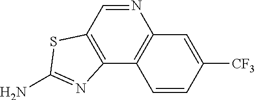 [SMILES: Nc1nc2c(cnc3cc(C(F)(F)F)ccc32)s1]

To a stirred solution of 7-(trifluoromethyl)-2,3-dihydroquinolin-4 (1H)-one (70.0 mg, 0.325 mmol, 1.00 equiv.) in DMSO (2.0 mL) was added thiourea (25 mg, 0.325 mmol, 1.00 equiv.), I₂ (29 mg, 0.114 mmol, 0.35 equiv.), TsOH (160 mg, 1.625 mmol, 5.00 equiv.) at room temperature. The resulting mixture was stirred for 2 h at 75° C. under air atmosphere. The reaction was cooled down to room temperature, then poured it into ice water. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (13 mg, 15% yield) as a yellow solid. ESI-MS m/z=269.86 [M+H]⁺; Calculated MW: 269.02

### Example 72D: 4,6-dimethoxy-N—(7-(trifluoromethyl) thiazolo[5,4-c]quinolin-2-yl)pyrimidine-5-carboxamide

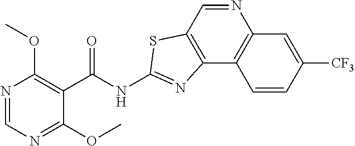 [SMILES: COc1ncnc(OC)c1C(=O)Nc1nc2c(cnc3cc(C(F)(F)F)ccc32)s1]

To a stirred solution of 4,6-dimethoxypyrimidine-5-carboxylic acid (12 mg, 0.067 mmol, 1.50 equiv.) in DCM (1.0 mL) was added (COCl)₂ (28.0 mg, 0.223 mmol, 5.00 equiv.) and DMF (1 mg) in DCM (0.1 mL) in portions at 0° C. The resulting mixture was stirred for 3 h at room temperature under argon atmosphere, concentrated under reduced pressure and dissolved with THF (1 mL). To a stirred solution of 7-(trifluoromethyl) thiazolo[5,4-c]quinolin-2-amine (12.0 mg, 0.045 mmol, 1.00 equiv.) in THF (1.0 mL) was added KOH (13.0 mg, 0.223 mmol, 5.00 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (5.0 mg, 25% yield) as a white solid. ESI-MS m/z=435.98 [M+H]⁺; Calculated MW: 435.06; ¹H NMR (400 MHz, Chloroform-d) δ 10.76 (s, 1H), 9.40 (s, 1H), 8.66 (d, J=8.6 Hz, 1H), 8.51 (d, J=1.7 Hz, 1H), 8.49 (s, 1H), 7.83 (dd, J=8.6, 1.8 Hz, 1H), 4.17 (s, 6H).

### Example 73: 4,6-dimethoxy-N—(1′-(methylsulfonyl)-7-(trifluoromethyl)spiro[chromeno[4,3-d]thiazole-4,4′-piperidin]-2-yl)pyrimidine-5-carboxamide

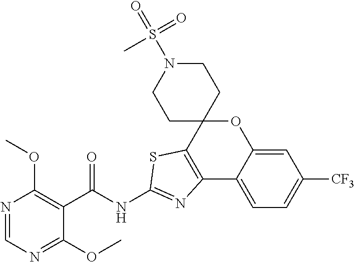 [SMILES: COc1ncnc(OC)c1C(=O)Nc1nc2c(s1)C1(CCN(S(C)(=O)=O)CC1)Oc1cc(C(F)(F)F)ccc1-2]

To a solution of methanesulfonic acid (11.5 mg, 0.12 mmol, 1.5 equiv.) in dichloromethane (0.5 mL) and catalytic amount of DMF, was added oxalyl chloride (50.8 mg, 0.40 mmol, 5.0 equiv.) dropwise at 0° C., and the mixture was stirred for 2 hours at room temperature. After concentration, the mixture was dissolved in tetrahydrofuran (0.5 mL). To a suspension of 4,6-dimethoxy-N—(7-(trifluoromethyl)spiro[chromeno[4,3-d]thiazole-4,4′-piperidin]-2-yl)pyrimidine-5-carboxamide (50 mg, 0.08 mmol, 1.0 equiv.) in THF (1.0 mL) were added KOH (13.4 mg, 0.24 mmol, 3.0 equiv.) and the acyl chloride/THF at 0° C. under argon atmosphere, then the mixture was stirred overnight at room temperature. Then the mixture was removed tetrahydrofuran under vacuum. The resulting residue was extracted with ethyl acetate (3×5 mL). The combined organic layers were washed with brine, dried over anhydrous Na₂SO₄. The resulting mixture was concentrated under vacuum. The crude product was purified by Prep-TLC (PE:EA=1:1) to afford the title compound (11.2 mg, 16% yield) as a white solid. ESI-MS m/z=585.93 [M+H]⁺; Calculated MW: 585.09. ¹H NMR (400 MHz, Chloroform-d) δ 10.88 (s, 1H), 8.35 (s, 1H), 7.65 (dd, J=8.3, 1.0 Hz, 1H), 7.21 (h, J=1.7 Hz, 2H), 4.01 (s, 6H), 3.83-3.65 (m, 2H), 3.25 (dd, J=12.2, 2.5 Hz, 2H), 2.89 (s, 3H), 2.45-2.33 (m, 2H), 2.08 (td, J=13.2, 4.8 Hz, 2H)

### Example 74: N—(1-(2,2-dimethylbutanoyl)-7′-(trifluoromethyl)spiro[azetidine-3,4′-chromeno[4,3-d]thiazol]-2′-yl)-4,6-dimethoxypyrimidine-5-carboxamide

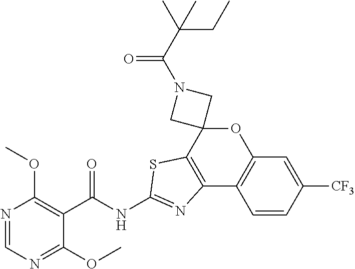 [SMILES: CCC(C)(C)C(=O)N1CC2(C1)Oc1cc(C(F)(F)F)ccc1-c1nc(NC(=O)c3c(OC)ncnc3OC)sc12]

To a stirred solution of 4,6-dimethoxy-N—(7′-(trifluoromethyl)spiro[azetidine-3,4′-chromeno[4,3-d]thiazol]-2′-yl)pyrimidine-5-carboxamide (8.0 mg, 0.017 mmol, 1.00 equiv.) in DCM (1.00 mL) was added TEA (17 mg, 0.167 mmol, 10.00 equiv.), 2,2-dimethylbutanoyl chloride (2.2 mg, 0.017 mmol, 1.00 equiv.) in DCM (0.2 mL) in portions at 0° C. The resulting mixture was stirred for 1 h at 0° C. The reaction was diluted with DCM at 0° C., then washed with water (2×10 mL), dried over anhydrous Na₂SO₄. After filtration, the filtrate was concentrated under reduced pressure. The residue was purified by prep-TLC (EA/PE=(2:1)) to afford the title compound (5 mg, 52% yield) as a white solid. ESI-MS m/z=577.98 [M+H]⁺; Calculated MW: 577.16; ¹H NMR (400 MHz, Chloroform-d) δ 10.74 (s, 1H), 8.41 (s, 1H), 7.68 (d, J=7.7 Hz, 1H), 7.23 (d, J=8.6 Hz, 2H), 4.57 (d, J=63.9 Hz, 4H), 4.07 (s, 6H), 1.56 (q, J=7.9, 7.4 Hz, 2H), 1.19 (s, 6H), 0.93 (t, J=7.4 Hz, 3H).

### Example 75: N—(1-benzoyl-7′-(trifluoromethyl)spiro[azetidine-3,4′-chromeno[4,3-d]thiazol]-2′-yl)-4,6-dimethoxypyrimidine-5-carboxamide

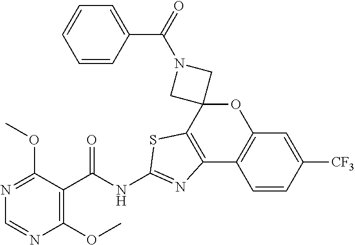 [SMILES: COc1ncnc(OC)c1C(=O)Nc1nc2c(s1)C1(CN(C(=O)c3ccccc3)C1)Oc1cc(C(F)(F)F)ccc1-2]

To a suspension of 4,6-dimethoxy-N—(7′-(trifluoromethyl)spiro[azetidine-3,4′-chromeno[4,3-d]thiazol]-2′-yl)pyrimidine-5-carboxamide (15 mg, 0.031 mmol, 1.0equiv.) in dichloromethane (0.2 mL) was added triethylamine (9.4 mg, 0.093 mmol, 3.0equiv.) and benzoyl chloride (6.6 mg, 0.047 mmol, 1.5equiv.) dropwise at 0° C., and the mixture was stirred for 2 hours at room temperature. The resulting residue was extracted with dichloromethane (3×2 mL). The combined organic layers were washed with brine, dried over anhydrous Na₂SO₄. The resulting mixture was concentrated under vacuum. The crude product was purified by Prep-TLC (PE:EA=1:1) to afford the title compound (8.8 mg, 48%) as a white solid. ESI-MS m/z=583.91 [M+H]⁺; Calculated MW: 583.11. ¹H NMR (400 MHz, Chloroform-d) δ 10.72 (s, 1H), 8.41 (s, 1H), 7.74-7.65 (m, 3H), 7.52-7.41 (m, 3H), 7.22 (t, J=1.3 Hz, 2H), 4.74 (d, J=10.7 Hz, 2H), 4.66-4.53 (m, 2H), 4.07 (s, 6H).

### Example 76: N—(1-(furan-2-carbonyl)-7′-(trifluoromethyl)spiro[azetidine-3,4′-chromeno[4,3-d]thiazol]-2′-yl)-4,6-dimethoxypyrimidine-5-carboxamide

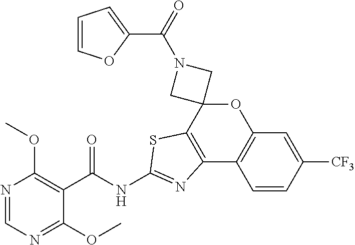 [SMILES: COc1ncnc(OC)c1C(=O)Nc1nc2c(s1)C1(CN(C(=O)c3ccco3)C1)Oc1cc(C(F)(F)F)ccc1-2]

To a suspension of furan-2-carboxylic acid (5.3 mg, 0.047 mmol, 1.5 equiv.) in dichloromethane (0.2 mL) and catalytic amount of DMF, was added oxalyl chloride (19.7 mg, 0.155 mmol, 5.0 equiv.) dropwise at 0° C., and the mixture was stirred for 2 hours at room temperature. After concentration, the mixture was dissolved in dichloromethane (0.2 mL). 4,6-dimethoxy-N—(7′-(trifluoromethyl)spiro[azetidine-3,4′-chromeno[4,3-d]thiazol]-2′-yl)pyrimidine-5-carboxamide (15.0 mg, 0.031 mmol, 1.0 equiv.), Et₃N (9.4 mg, 0.093 mmol, 3.0 equiv.) were added at 0° C., and the mixture was stirred overnight at room temperature. The resulting residue was extracted with dichloromethane (3×2 mL). The combined organic layers were washed with brine, dried over anhydrous Na₂SO₄. The resulting mixture was concentrated under vacuum. The crude product was purified by Prep-TLC (PE:EA=1:1) to afford the title compound (9.7 mg, 54%) as a white solid. ESI-MS m/z=573.93 [M+H]⁺; Calculated MW: 573.09. ¹H NMR (400 MHz, Chloroform-d) δ 10.84 (s, 1H), 8.38 (s, 1H), 7.67 (dt, J=7.6, 1.0 Hz, 1H), 7.50 (dd, J=1.8, 0.8 Hz, 1H), 7.22 (t, J=1.2 Hz, 2H), 7.16 (dd, J=3.6, 0.8 Hz, 1H), 6.52 (dd, J=3.5, 1.7 Hz, 1H), 4.77 (dd, J=135.4, 60.6 Hz, 4H), 4.04 (s, 6H).

### Example 77: butyl 2′-(4,6-dimethoxypyrimidine-5-carboxamido)-7′-(trifluoromethyl)spiro[azetidine-3,4′-chromeno[4,3-d]thiazole]-1-carboxylate

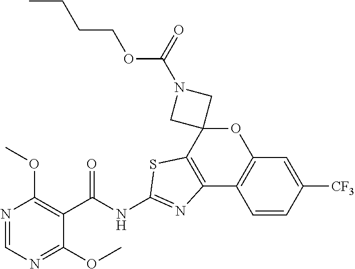 [SMILES: CCCCOC(=O)N1CC2(C1)Oc1cc(C(F)(F)F)ccc1-c1nc(NC(=O)c3c(OC)ncnc3OC)sc12]

### Example 77A: butyl pyridin-2-yl carbonate

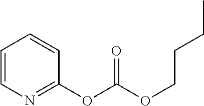

To a stirred solution of butan-1-ol (38.00 mg, 0.509 mmol, 1.10 equiv.) in THF (2.00 mL) was added NaH (29.00 mg, 0.694 mmol, 1.50 equiv., 60%) in portions at 0° C. The resulting mixture was stirred for 0.5 h at 0° C. under argon. To the above mixture was added di(pyridin-2-yl) carbonate (100 mg, 0.463 mmol, 1.00 equiv.) in THF (1 mL) dropwise at 0° C. The resulting mixture was stirred for additional 0.5 h at 0° C. The reaction was quenched with NH₄Cl(aq.), extracted with EtOAc (3×20 mL). The combined organic layers were washed with brine (2×20 mL), dried over anhydrous Na₂SO₄. After filtration, the filtrate was concentrated under reduced pressure. The residue was purified by prep-TLC (PE/EtOAc (2:1)) to afford the title compound (30 mg, 33% yield) as a yellow solid. ESI-MS m/z=195.93 [M+H]⁺; Calculated MW: 195.09

### Example 77B: butyl 2′-(4,6-dimethoxypyrimidine-5-carboxamido)-7′-(trifluoromethyl)spiro[azetidine-3,4′-chromeno[4,3-d]thiazole]-1-carboxylate

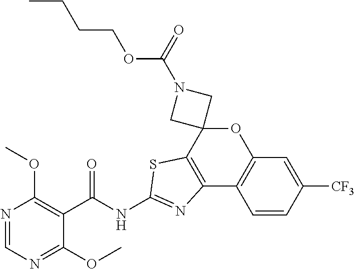 [SMILES: CCCCOC(=O)N1CC2(C1)Oc1cc(C(F)(F)F)ccc1-c1nc(NC(=O)c3c(OC)ncnc3OC)sc12]

To a stirred solution of 4,6-dimethoxy-N—(7′-(trifluoromethyl)spiro[azetidine-3,4′-chromeno[4,3-d]thiazol]-2′-yl)pyrimidine-5-carboxamide (10 mg, 0.021 mmol, 1.00 equiv.) in DCM (1.00 mL) was added DIEA (27.0 mg, 0.208 mmol, 10.00 equiv.) and butyl pyridin-2-yl carbonate (6 mg, 0.031 mmol, 1.50 equiv.) in DCM (0.2 mL) in portions at 0° C. The resulting mixture was stirred for 16 h at room temperature under argon atmosphere. The reaction was diluted with DCM, washed with water (3×10 mL), brine (2×10 mL), dried over anhydrous Na₂SO₄. After filtration, the filtrate was concentrated under reduced pressure. The residue was purified by prep-TLC (PE/EtOAc (2:1)) to afford the title compound (6 mg, 50% yield) as an white solid. ESI-MS m/z=579.99 [M+H]⁺; Calculated MW: 579.14; 1H NMR (400 MHz, Chloroform-d) δ 10.66 (s, 1H), 8.42 (s, 1H), 7.70-7.65 (m, 1H), 7.24-7.21 (m, 2H), 4.53-4.45 (m, 2H), 4.34-4.29 (m, 2H), 4.11 (t, J=6.7 Hz, 2H), 4.07 (s, 6H), 1.68-1.61 (m, 2H), 1.45-1.34 (m, 2H), 0.95 (t, J=7.4 Hz, 3H).

### Example 78: ethyl 2′-(4,6-dimethoxypyrimidine-5-carboxamido)-7′-(trifluoromethyl)spiro[azetidine-3,4′-chromeno[4,3-d]thiazole]-1-carboxylate

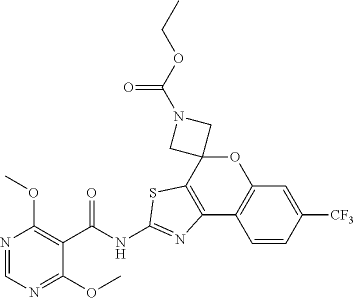 [SMILES: CCOC(=O)N1CC2(C1)Oc1cc(C(F)(F)F)ccc1-c1nc(NC(=O)c3c(OC)ncnc3OC)sc12]

### Example 78A: ethyl pyridin-2-yl carbonate

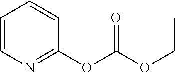

To a stirred solution of ethanol (18.00 mg, 0.388 mmol, 1.20 equiv.) in DCM (1.00 mL) was added DIEA (418.00 mg, 3.24 mmol, 10.00 equiv.) and di(pyridin-2-yl) carbonate (70 mg, 0.324 mmol, 1.00 equiv.) in DCM (1 mL) in portions at 0° C. The resulting mixture was stirred for 16 h at room temperature The reaction was quenched with NH₄Cl(aq.), extracted with EtOAc (3×20 mL). The combined organic layers were washed with brine (2×20 mL), dried over anhydrous Na₂SO₄. After filtration, the filtrate was concentrated under reduced pressure. The residue was purified by prep-TLC (PE/EtOAc (2:1)) to afford the title compound (30 mg, 55% yield) as a yellow solid. ESI-MS m/z=167.89 [M+H]⁺; Calculated MW: 167.06

### Example 78B: ethyl 2′-(4,6-dimethoxypyrimidine-5-carboxamido)-7′-(trifluoromethyl)spiro[azetidine-3,4′-chromeno[4,3-d]thiazole]-1-carboxylate

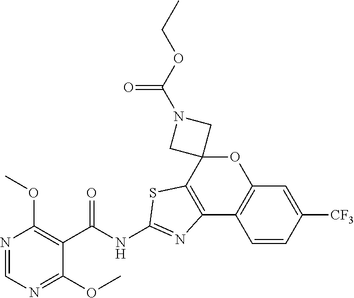 [SMILES: CCOC(=O)N1CC2(C1)Oc1cc(C(F)(F)F)ccc1-c1nc(NC(=O)c3c(OC)ncnc3OC)sc12]

To a stirred solution of 4,6-dimethoxy-N—(7′-(trifluoromethyl)spiro[azetidine-3,4′-chromeno[4,3-d]thiazol]-2′-yl)pyrimidine-5-carboxamide (10 mg, 0.021 mmol, 1.00 equiv.) in DCM (1.0 mL) was added DIEA (27.0 mg, 0.208 mmol, 10.00 equiv.) and ethyl pyridin-2-yl carbonate (6 mg, 0.031 mmol, 1.50 equiv.) in DCM (0.2 mL) in portions at 0° C. The resulting mixture was stirred for 16 h at room temperature under argon atmosphere. The reaction was diluted with DCM, washed with water (3×10 mL), brine (2×10 mL), dried over anhydrous Na₂SO₄. After filtration, the filtrate was concentrated under reduced pressure. The residue was purified by prep-TLC (PE/EtOAc (2:1)) to afford the title compound (10 mg, 86% yield) as a white solid. ESI-MS m/z=551.95 [M+H]⁺; Calculated MW: 551.11; ¹H NMR (400 MHz, Chloroform-d) δ 11.10 (s, 1H), 8.32 (s, 1H), 7.63-7.57 (m, 1H), 7.22 (d, J=1.6 Hz, 1H), 7.19 (ddd, J=8.0, 1.7, 0.8 Hz, 1H), 4.50 (dd, J=9.9, 1.0 Hz, 2H), 4.31 (dd, J=9.8, 1.1 Hz, 2H), 4.17 (q, J=7.1 Hz, 2H), 4.00 (s, 6H), 1.28 (t, J=7.1 Hz, 3H).

### Example 79: N—(4,4-diethyl-7-(trifluoromethyl)-4H-chromeno[4,3-d]thiazol-2-yl)-2-hydroxy-4,6-dimethoxypyrimidine-5-carboxamide

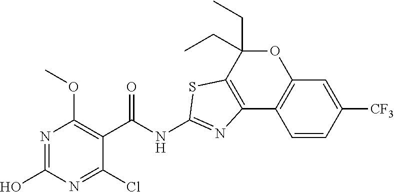 [SMILES: CCC1(CC)Oc2cc(C(F)(F)F)ccc2-c2nc(NC(=O)c3c(Cl)nc(O)nc3OC)sc21]

### Example 79A: 4,6-dimethoxy-2-(methylthio)pyrimidine-5-carboxylic acid

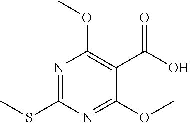

To a stirred solution of 4,6-dichloro-2-(methylthio)pyrimidine-5-carboxylic acid (100.0 mg, 0.420 mmol, 1.00 equiv.) in MeOH (2.0 mL) was added KOH (94.0 mg, 1.680 mmol, 4.00 equiv.) in portions at 0° C. The resulting mixture was stirred for additional 16 h at 70° C. The reaction was diluted with water (20 mL), adjusted pH to 5 with 2 M HCl in dioxane. The resulting mixture was filtered, the filter cake was washed with EA (3×20 mL), dried in the oven. This resulted in the title compound (125 mg, crude) as an off-white solid. ESI-MS m/z=230.85 [M+H]⁺; Calculated MW: 230.04.

### Example 79B: N—(4,4-diethyl-7-(trifluoromethyl)-4H-chromeno[4,3-d]thiazol-2-yl)-4,6-dimethoxy-2-(methylthio)pyrimidine-5-carboxamide

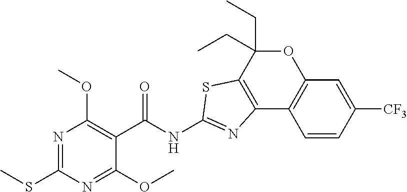 [SMILES: CCC1(CC)Oc2cc(C(F)(F)F)ccc2-c2nc(NC(=O)c3c(OC)nc(SC)nc3OC)sc21]

To a stirred solution of 4,6-dimethoxy-2-(methylthio)pyrimidine-5-carboxylic acid (42.0 mg, 0.183 mmol, 1.20 equiv.) in DCM (1.0 mL) was added (COCl)₂ (97.0 mg, 0.762 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (50.00 mg, 0.152 mmol, 1.00 equiv.) in THF (1.00 mL) was added KOH (26.00 mg, 0.457 mmol, 3.00 equiv.) in portions at 0° C. The resulting mixture was stirred for 0.5 h at 0° C. under argon atmosphere. To the above mixture was added the acyl chloride/THE dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (40 mg, 48% yield) as a light yellow solid. ESI-MS m/z=540.92 [M+H]⁺; Calculated MW: 540.11.

### Example 79C: N—(4,4-diethyl-7-(trifluoromethyl)-4H-chromeno[4,3-d]thiazol-2-yl)-4,6-dimethoxy-2-(methylsulfonyl)pyrimidine-5-carboxamide

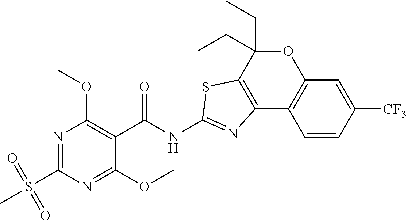 [SMILES: CCC1(CC)Oc2cc(C(F)(F)F)ccc2-c2nc(NC(=O)c3c(OC)nc(S(C)(=O)=O)nc3OC)sc21]

To a stirred solution of N—(4,4-diethyl-7-(trifluoromethyl)-4H-chromeno[4,3-d]thiazol-2-yl)-4,6-dimethoxy-2-(methylthio)pyrimidine-5-carboxamide (20.0 mg, 0.037 mmol, 1.00 equiv.) in DCM (0.5 mL) was added m-CPBA (26.0 mg, 0.148 mmol, 4.00 equiv.) in DCM (0.5 mL) in portions at 0° C. The resulting mixture was stirred for 16 h at room temperature under argon atmosphere. The reaction was diluted with DCM, washed with NaHCO₃ (aq.) (2×10 mL), brine (1×10 mL), dried over anhydrous Na₂SO₄. After filtration, the filtrate was concentrated under reduced pressure. The residue was purified by prep-TLC (PE/EtOAc (2:1)) to afford the title compound (14 mg, 66% yield) as a yellow solid. ESI-MS m/z=572.93 [M+H]⁺; Calculated MW: 572.10.

### Example 79D: N—(4,4-diethyl-7-(trifluoromethyl)-4H-chromeno[4,3-d]thiazol-2-yl)-2-hydroxy-4,6-dimethoxypyrimidine-5-carboxamide

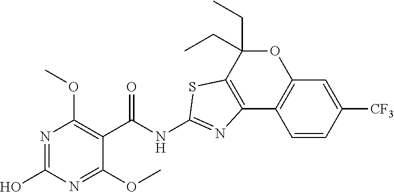 [SMILES: CCC1(CC)Oc2cc(C(F)(F)F)ccc2-c2nc(NC(=O)c3c(OC)nc(O)nc3OC)sc21]

To a stirred solution of N—(4,4-diethyl-7-(trifluoromethyl)-4H-chromeno[4,3-d]thiazol-2-yl)-4,6-dimethoxy-2-(methylsulfonyl)pyrimidine-5-carboxamide (7.0 mg, 0.012 mmol, 1.00 equiv.) in THF (0.5 mL) was added KOH (2.00 mg, 0.037 mmol, 3.00 equiv.) in H₂O (0.5 mL) in portions at 0° C. The resulting mixture was stirred for 16 h at room temperature under argon atmosphere. The reaction was adjusted pH to 5 with HCl (1 M), extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (DCM/MeOH (10:1)) to afford the title compound (4 mg, 65% yield) as a white solid. ESI-MS m/z=510.95 [M+H]⁺; Calculated MW: 510.12; 1H NMR (400 MHz, Chloroform-d) δ 11.04 (s, 1H), 7.68 (d, J=7.9 Hz, 1H), 7.13 (d, J=13.0 Hz, 2H), 3.97 (s, 6H), 2.10-1.64 (m, 4H), 0.96 (t, J=7.3 Hz, 6H).

### Example 80:2-amino-N—(4,4-diethyl-7-(trifluoromethyl)-4H-chromeno[4,3-d]thiazol-2-yl)-4,6-dimethoxypyrimidine-5-carboxamide

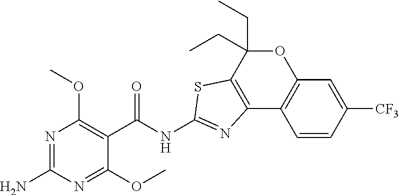 [SMILES: CCC1(CC)Oc2cc(C(F)(F)F)ccc2-c2nc(NC(=O)c3c(OC)nc(N)nc3OC)sc21]

To a stirred solution of N—(4,4-diethyl-7-(trifluoromethyl)-4H-chromeno[4,3-d]thiazol-2-yl)-4,6-dimethoxy-2-(methylsulfonyl)pyrimidine-5-carboxamide (7.0 mg, 0.012 mmol, 1.00 equiv.) in dioxane (1 mL) was added NH₃H₂O (1 mL) at room temperature. The resulting mixture was stirred for 2 h at 70° C. under argon atmosphere. The reaction was cooled down to room temperature, diluted with water, extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (1:1)) to afford the title compound (4 mg, 65% yield) as a white solid. ESI-MS m/z=509.96 [M+H]⁺; Calculated MW: 509.13; ¹H NMR (400 MHz, Chloroform-d) δ 10.64 (s, 1H), 7.67 (d, J=7.9 Hz, 1H), 7.15-7.07 (m, 2H), 5.16 (s, 2H), 4.01 (s, 6H), 1.98 (dt, J=14.7, 7.4 Hz, 2H), 1.87 (dq, J=14.6, 7.4 Hz, 2H), 0.95 (t, J=7.4 Hz, 6H).

### Example 81: N—(4,4-diethyl-7-ethynyl-4H-chromeno[4,3-d]thiazol-2-yl)-4,6-dimethoxypyrimidine-5-carboxamide

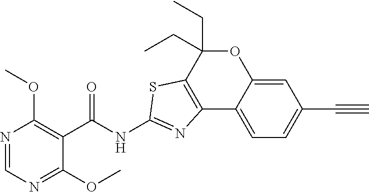 [SMILES: C#Cc1ccc2c(c1)OC(CC)(CC)c1sc(NC(=O)c3c(OC)ncnc3OC)nc1-2]

### Example 81A: N—(4,4-diethyl-7-((triisopropylsilyl) ethynyl)-4H-chromeno[4,3-d]thiazol-2-yl)-4,6-dimethoxypyrimidine-5-carboxamide

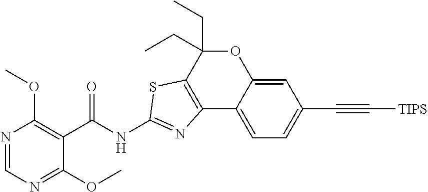

N—(7-bromo-4,4-diethyl-4H-chromeno[4,3-d]thiazol-2-yl)-4,6-dimethoxypyrimidine-5-carboxamide (20 mg, 39.57 μmol, 1 equiv.), ethynyltriisopropylsilane (18.04 mg, 99.93 μmol, 98.93 μl, 2.5 equiv.), TEA (40.05 mg, 395.74 μmol, 55.24 μl, 10equiv.) and CuI(1.51 mg, 7.91 μmol, 0.2 equiv.) were added to a flame-dried tube. Toluene (0.4 mL) and Pd(PPh₃)₄ (9.15 mg, 7.91 μmol, 0.2 equiv.) were added to the mixture. The tube was evacuated and back-filled with Ar. The mixture was stirred at 80° C. for 3 hours. The mixture was concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA=3:1) to afford the title compound (10 mg, 41) as yellow solid. ¹H NMR (400 MHz, Chloroform-d) δ 12.09 (s, 1H), 8.18 (s, 1H), 7.24 (d, J=7.9 Hz, 1H), 6.96 (d, J=1.5 Hz, 1H), 6.86 (dd, J=7.8, 1.5 Hz, 1H), 3.86 (s, 6H), 1.92 (ddt, J=36.3, 14.3, 7.3 Hz, 4H), 1.11 (s, 18H), 0.99 (t, J=7.4 Hz, 6H).

### Example 81B: N—(4,4-diethyl-7-ethynyl-4H-chromeno[4,3-d]thiazol-2-yl)-4,6-dimethoxypyrimidine-5-carboxamide

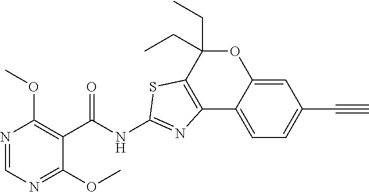 [SMILES: C#Cc1ccc2c(c1)OC(CC)(CC)c1sc(NC(=O)c3c(OC)ncnc3OC)nc1-2]

N—(4,4-diethyl-7-((triisopropylsilyl) ethynyl)-4H-chromeno[4,3-d]thiazol-2-yl)-4,6-dimethoxypyrimidine-5-carboxamide (10 mg, 22.25 μmol, 1 equiv.) was added to a flame-dried tube. THF (0.2 mL) was added to the mixture and cooled the mixture to 0° C. TBAF (26.7 μl, 26.70 μmol, 1.2 equiv.) was added to the mixture. The mixture was stirred at 0 to rt for 1 hour. The mixture was concentrated under reduce pressure then quenched with ice-water (5 mL). The aqueous was extracted with EA (10 mL×2). The combine organic layers were washed with brine (20 mL×2), dried over Na₂SO₄, filtrated and concentrated under reduce pressure. The crude product was purified by prep-TLC (PE:EA-3:1) to afford the title compound (5 mg, 50%) as white-off solid. ESI-MS m/z=450.93 [M+H]⁺; Calculated MW: 450.51 ¹H NMR (400 MHz, Chloroform-d) δ 11.80 (s, 1H), 8.21 (s, 1H), 7.29 (d, J=7.8 Hz, 1H), 6.98 (d, J=1.5 Hz, 1H), 6.90 (dd, J=7.8, 1.5 Hz, 1H), 3.90 (s, 6H), 3.08 (s, 1H), 1.93 (ddq, J=45.8, 14.6, 7.4 Hz, 4H), 0.99 (t, J=7.4 Hz, 6H).

### Example 82: 4,6-dimethoxy-N—(8-(trifluoromethyl)-4,5-dihydrobenzo[2,3]oxepino[4,5-d]thiazol-2-yl)pyrimidine-5-carboxamide

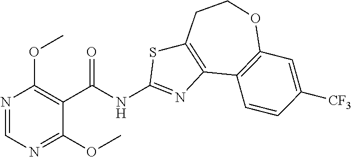 [SMILES: COc1ncnc(OC)c1C(=O)Nc1nc2c(s1)CCOc1cc(C(F)(F)F)ccc1-2]

### Example 82A: methyl 2-(4-methoxy-4-oxobutoxy)-4-(trifluoromethyl)benzoate

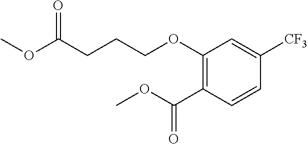 [SMILES: COC(=O)CCCOc1cc(C(F)(F)F)ccc1C(=O)OC]

To a stirred solution of methyl 4-bromobutanoate (296 mg, 1.6352 mmol, 1.2 equiv.), K₂CO₃ (263.7 mg, 1.9078 mmol, 1.4 equiv.) and KI (22.6 mg, 0.1363 mmol, 0.1 equiv.) in acetone (10 mL) was added methyl 2-hydroxy-4-(trifluoromethyl)benzoate (300 mg, 1.3627 mmol, 1.00 equiv.) at 0° C., and the mixture was stirred overnight at 60° C. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel chromatography (PE:EA=20:1) to afford the title compound (321 mg, 74%) as a colorless oil. ¹H NMR (400 MHz, Chloroform-d) δ 7.85 (d, J=8.1 Hz, 1H), 7.22 (d, J=8.1 Hz, 1H), 7.15 (s, 1H), 4.12 (td, J=6.0, 1.6 Hz, 2H), 3.90 (d, J=1.6 Hz, 3H), 3.68 (d, J=1.8 Hz, 3H), 2.60 (td, J=7.2, 1.6 Hz, 2H), 2.17 (td, J=7.4, 5.8 Hz, 2H)

### Example 82B: methyl 5-oxo-8-(trifluoromethyl)-2,3,4,5-tetrahydrobenzo[b]oxepine-4-carboxylate

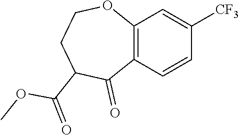 [SMILES: COC(=O)C1CCOc2cc(C(F)(F)F)ccc2C1=O]

To a suspension of NaH (187 mg, 4.6836 mmol, 3 equiv.) in DMF (5 mL) was added methyl 2-(4-methoxy-4-oxobutoxy)-4-(trifluoromethyl)benzoate (500 mg, 1.5612 mmol, 1equiv.) in DMF (5 mL) at 0° C. and the mixture was stirred for 3 h at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=15:1) to afford the title compound (130 mg, 29%) as a colorless oil. ¹H NMR (400 MHz, Chloroform-d) δ 7.34 (dd, J=8.3, 1.9 Hz, 2H), 7.27 (d, J=1.9 Hz, 1H), 4.41 (dd, J=5.7, 5.0 Hz, 2H), 3.85 (s, 3H), 3.74 (s, 1H), 2.75-2.67 (m, 2H).

### Example 82C: 8-(trifluoromethyl)-3,4-dihydrobenzo[b]oxepin-5 (2H)-one

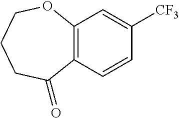 [SMILES: O=C1CCCOc2cc(C(F)(F)F)ccc21]

A suspension of methyl 5-oxo-8-(trifluoromethyl)-2,3,4,5-tetrahydrobenzo[b]oxepine-4-carboxylate (130 mg, 0.450 mmol, 1.00 equiv.) in H₂O (1 mL) and DMF (3 mL) was stirred overnight at 153° C. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=50:1) to afford the title compound (63.5 mg, 61%) as a yellow solid. ¹H NMR (400 MHz, Chloroform-d) δ 7.84 (d, J=8.3 Hz, 1H), 7.33 (d, J=6.9 Hz, 2H), 4.28 (t, J=6.6 Hz, 2H), 2.92 (t, J=7.0 Hz, 2H), 2.26 (p, J=6.8 Hz, 2H).

### Example 82D: 8-(trifluoromethyl)-4,5-dihydrobenzo[2,3]oxepino[4,5-d]thiazol-2-amine

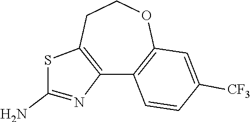 [SMILES: Nc1nc2c(s1)CCOc1cc(C(F)(F)F)ccc1-2]

To a suspension of 8-(trifluoromethyl)-3,4-dihydrobenzo[b]oxepin-5 (2H)-one (60 mg, 0.261 mmol, 1.00 equiv.) and TsOH-H₂O (5 mg, 0.0261 mmol, 0.10 equiv.) in toluene (2 mL), pyrrolidine (92.8 mg, 1.305 mmol, 5.00 equiv.) was added at 0° C., and the mixture was stirred for 2 h at 120° C. Then the reaction mixture was concentrated in vacuo and dissolved in MeOH (2 mL). S (21 mg, 0.6525 mmol, 2.5 equiv.) was added at 0° C. and the mixture was stirred for 5 mins at 0° C. Then 51% w/w % cyanamide (53.8 mg, 0.6525 mmol, 2.5 equiv.) was added at 0° C. and the mixture was stirred overnight at room temperature. The resulting mixture was concentrated under vacuum and was extracted with EA (2×5 mL). The combined organic layers were washed with brine (1×10 mL), dried over anhydrous Na₂SO₄. After filtration, the filtrate was concentrated under reduced pressure. The crude product was purified by silica gel column chromatography (PE:EA=10:1) to afford the title compound (18.3 mg, 25%) as a yellow solid. ¹H NMR (400 MHz, Chloroform-d) δ 8.34 (d, J=8.3 Hz, 1H), 7.33-7.28 (m, 1H), 7.24 (s, 1H), 4.80 (s, 2H), 4.34 (t, J=5.1 Hz, 2H), 3.18 (t, J=5.1 Hz, 2H).

### Example 82E: 4,6-dimethoxy-N—(8-(trifluoromethyl)-4,5-dihydrobenzo[2,3]oxepino[4,5-d]thiazol-2-yl)pyrimidine-5-carboxamide

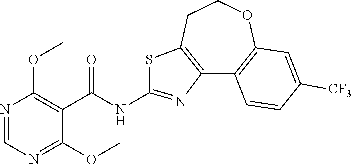 [SMILES: COc1ncnc(OC)c1C(=O)Nc1nc2c(s1)CCOc1cc(C(F)(F)F)ccc1-2]

To a suspension of 4,6-dimethoxypyrimidine-5-carboxylic acid (13.1 mg, 0.0839 mmol, 1.20 equiv.) in CH₂Cl₂ (0.5 mL) and catalytic amount of DMF, oxalyl chloride (22.2 mg, 0.1748 mmol, 2.50 equiv.) was added dropwise at 0° C., and the mixture was stirred for 2 h at room temperature. Then the reaction mixture was concentrated in vacuo and dissolved in THF (0.5 mL). 8-(trifluoromethyl)-4,5-dihydrobenzo[2,3]oxepino[4,5-d]thiazol-2-amine (18.3 mg, 0.0699 mmol, 1.0 equiv.), KOH (11.8 mg, 0.210 mmol, 3.0 equiv.) and THF (0.5 mL) were added and stirred for 10 min at 0° C., then the mixture was stirred overnight at room temperature. Then the resulting mixture was extracted with EA (2×5 mL) and washed with sat. NaHCO₃ (1×5 mL). The combined organic layers were washed with brine (1×10 mL), dried over anhydrous Na₂SO₄. After filtration, the filtrate was concentrated under reduced pressure. The crude product was purified by TLC (PE:EA=3:1) to afford the title compound (3 mg, 9.5%) as a white solid. ESI-MS m/z=452.88 [M+H]⁺; Calculated MW: 452.08. ¹H NMR (400 MHz, Chloroform-d) δ 10.40 (s, 1H), 8.43 (s, 1H), 8.34 (d, J=8.3 Hz, 1H), 7.34-7.26 (m, 2H), 4.38 (t, J=5.0 Hz, 2H), 4.04 (s, 6H), 3.33 (t, J=5.0 Hz, 2H).

### Example 83: N—(4,4-diethyl-8-(trifluoromethyl)-4H-chromeno[4,3-d]thiazol-2-yl)-4,6-dimethoxypyrimidine-5-carboxamide

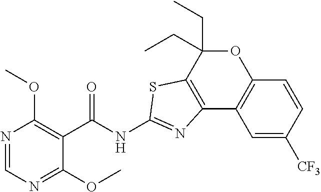 [SMILES: CCC1(CC)Oc2ccc(C(F)(F)F)cc2-c2nc(NC(=O)c3c(OC)ncnc3OC)sc21]

### Example 83A: 1-(2-hydroxy-5-(trifluoromethyl)phenyl)ethan-1-one

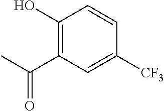

2-bromo-4-(trifluoromethyl) phenol (1 g, 4.15 mmol, 1 equiv.), Bu₃Sn (1-ethoxyvinyl) (1.8 g, 4.98 mmol, 1.2equiv.) and Pd(PPh₃)₂Cl₂ (291.24 mg, 414.93 μmol, 0.1 equiv.) were added to a flame-dried tube. Dioxane (10 mL) was added to the mixture. The tube was evacuated and back-filled with Ar. for 3 times. The mixture was stirred at 95° C. for 16 hours. HCl (4.15 mL, 4.15 mmol, 1 eq, 1 mmol/mL) was added to the mixture and the reaction was stirred at 50° C. for 1 hour. After cooled to rt, to the mixture was added sat KF aqueous solution, stirred 0.5 hour and filtrated. The aqueous layer was extracted with DCM (10 mL×2). The combine organic layers were washed with brine (20 mL×2), dried over Na₂SO₄, filtrated and concentrated under reduce pressure. The crude product was purified by silica gel column (PE:EA=100:1) to afford the title compound (333 mg, 35%) as yellow oil. ¹H NMR (400 MHz, Chloroform-d) δ 12.55 (s, 1H), 7.98 (dd, J=2.3, 1.0 Hz, 1H), 7.69 (dd, J=8.8, 2.3 Hz, 1H), 7.07 (d, J=8.8 Hz, 1H), 2.68 (s, 3H).

### Example 83B: 2,2-diethyl-6-(trifluoromethyl) chroman-4-one

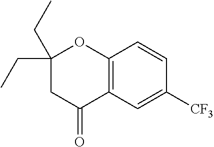 [SMILES: CCC1(CC)CC(=O)c2cc(C(F)(F)F)ccc2O1]

To a stirred solution of 1-(2-hydroxy-5-(trifluoromethyl)phenyl)ethan-1-one (330 mg, 1.62 mmol, 1.00 equiv.) and pentan-3-one (208.9 mg, 2.42 mmol, 1.5 equiv.) in MeOH (6 mL) was added pyrrolidine (229.9 mg, 3.23 mmol, 2.00 equiv.) at 0° C. The mixture was stirred at 50° C. for 16 hours. The mixture was quenched with ice-water and extracted with EA (20 mL×2). The combined organic layers were washed twice by brine (20 mL×2), dried over Na₂SO₄, filtrated, and concentrated under reduce pressure. The residue was purified by prep-TLC (PE:EA=3:1) to get the title compound (225 mg, 51%) as yellow oil. ¹H NMR (400 MHz, Chloroform-d) δ 8.20-8.03 (m, 1H), 7.65 (ddd, J=8.8, 2.5, 0.7 Hz, 1H), 7.16-6.90 (m, 1H), 2.74 (s, 2H), 1.88-1.65 (m, 4H), 0.91 (t, J=7.5 Hz, 6H).

### Example 83C: 4,4-diethyl-8-(trifluoromethyl)-4H-chromeno[4,3-d]thiazol-2-amine

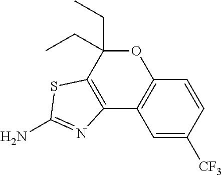 [SMILES: CCC1(CC)Oc2ccc(C(F)(F)F)cc2-c2nc(N)sc21]

To a suspension of 2,2-diethyl-6-(trifluoromethyl) chroman-4-one (1.6 g, 5.88 mmol, 1.00 equiv.) and TsOH-H₂O (111.8 mg, 587.66 μmol, 0.10 equiv.) in toluene (20 mL), pyrrolidine (2.09 g, 29.38 mmol, 5.00 equiv.) was added at room temperature, and the mixture was stirred for 2 h at 110° C. Then the reaction mixture was concentrated in vacuo and dissolved in MeOH (20 mL). S (471.01 mg, 14.69 mmol, 2.5 equiv.) was added at 0° C. and the mixture was stirred for 5 mins at 0° C. Then 51% w/w % cyanamide (1.21 g, 14.69 mmol, 2.5 equiv.) was added at 0° C. and the mixture was stirred overnight at room temperature. The resulting mixture was concentrated under vacuum and was extracted with EA (2×30 mL). The combined organic layers were washed with brine (1×30 mL), dried over anhydrous Na₂SO₄. After filtration, the filtrate was concentrated under reduced pressure. The crude product was purified by silica gel column chromatography (PE:EA=3:1) to afford the title compound (210 mg, 11%) as a yellow solid. ¹H NMR (400 MHz, Chloroform-d) δ 7.79 (d, J=2.3 Hz, 1H), 7.33 (ddq, J=8.4, 2.3, 0.7 Hz, 1H), 6.87 (dd, J=8.4, 0.9 Hz, 1H), 5.10 (s, 2H), 1.95 (dq, J=14.7, 7.3 Hz, 2H), 1.77 (dq, J=14.7, 7.4 Hz, 2H), 0.94 (t, J=7.4 Hz, 6H).

### Example 83D: N—(4,4-diethyl-8-(trifluoromethyl)-4H-chromeno[4,3-d]thiazol-2-yl)-4,6-dimethoxypyrimidine-5-carboxamide

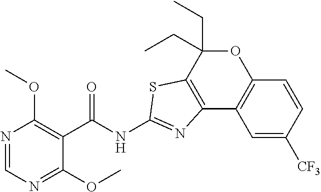 [SMILES: CCC1(CC)Oc2ccc(C(F)(F)F)cc2-c2nc(NC(=O)c3c(OC)ncnc3OC)sc21]

4,4-diethyl-8-(trifluoromethyl)-4H-chromeno[4,3-d]thiazol-2-amine (50 mg, 152.28 μmol, 1 equiv.) and 4,6-dimethoxypyrimidine-5-carboxylic acid (42.06 mg, 228.41 mmol, 1.5 equiv.) were added to a flame-dried three necked flask. The flask was evacuated and back-filled with Ar. ACN (1 mL) was added to the mixture and cool to 0° C. NMI (43.76 mg, 532.96 μmol, 3.5 equiv.) was added to the mixture at 0° C. TCFH (64.1 mg, 228.41 μmol, 1.5 equiv.) was dissolved in 0.5 mL ACN and added to the mixture at 0° C. The mixture was stirred at rt for 3 hours and stirred at 50° C. for 16 hours. The mixture was concentrated under reduce pressure then quenched with ice-water (10 mL). The aqueous layer was extracted with EA (10 mL×2). The combine organic layers were washed with brine (20 mL×2), dried over Na₂SO₄, filtrated and concentrated under reduce pressure. The crude product was purified by prep-TLC eluted with PE:EA=3:1 to afford the title compound (28 mg, 37%) as white solid. ESI-MS m/z=494.99 [M+H]⁺; Calculated MW: 494.48. ¹H NMR (400 MHz, Chloroform-d) δ 10.78 (s, 1H), 8.37 (s, 1H), 7.78 (d, J=2.3 Hz, 1H), 7.41-7.33 (m, 1H), 6.97-6.82 (m, 1H), 4.01 (s, 6H), 2.09-1.82 (m, 4H), 0.98 (t, J=7.4 Hz, 6H).

### Example 84: 4,6-dimethoxy-N—(6-(trifluoromethyl)-8H-indeno[1,2-d]thiazol-2-yl)pyrimidine-5-carboxamide

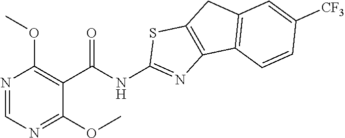 [SMILES: COc1ncnc(OC)c1C(=O)Nc1nc2c(s1)Cc1cc(C(F)(F)F)ccc1-2]

### Example 84A: 6-(trifluoromethyl)-8H-indeno[1,2-d]thiazol-2-amine

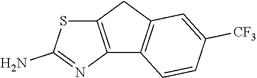 [SMILES: Nc1nc2c(s1)Cc1cc(C(F)(F)F)ccc1-2]

To a suspension of 5-(trifluoromethyl)-2,3-dihydro-1H-inden-1-one (20 mg, 0.0999 mmol, 1.00 equiv.) and TsOH-H₂O (1.9 mg, 0.0099 mmol, 0.10 equiv.) in n-hexane (2.5 mL), pyrrolidine (35.5 mg, 0.4996 mmol, 5.00 equiv.) was added at room temperature, and the mixture was stirred for 2 h at 80° C. Then the reaction mixture was concentrated in vacuo and dissolved in MeOH (1 mL), S (8.0 mg, 0.2498 mmol, 2.5 equiv.) was added at 0° C. and the mixture was stirred for 5 mins at 0° C. Then 51% w/w % cyanamide (6.3 mg, 0.0999 mmol, 2.5 equiv.) was added at 0° C. and the mixture was stirred overnight at room temperature. The resulting mixture was concentrated under vacuum and was extracted with EA (2×8 mL). The combined organic layers were washed with brine (1×5 mL), dried over anhydrous Na₂SO₄. After filtration, the filtrate was concentrated under reduced pressure. The crude product was purified by silica gel column chromatography (PE:EA=2:1) to afford the title compound (17 mg, 66%) as a yellow solid. ¹H NMR (400 MHz, Chloroform-d) δ 7.66 (s, 1H), 7.60 (s, 2H), 5.28 (s, 2H), 3.75 (s, 2H).

### Example 84B: 4,6-dimethoxy-N—(6-(trifluoromethyl)-8H-indeno[1,2-d]thiazol-2-yl)pyrimidine-5-carboxamide

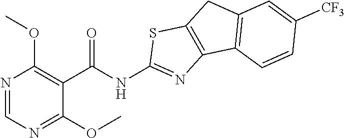 [SMILES: COc1ncnc(OC)c1C(=O)Nc1nc2c(s1)Cc1cc(C(F)(F)F)ccc1-2]

To a suspension of 6-(trifluoromethyl)-8H-indeno[1,2-d]thiazol-2-amine (17 mg, 0.066 mmol, 1.00 equiv.) and 4,6-dimethoxypyrimidine-5-carboxylic acid (18.3 mg, 0.099 mmol, 1.50 equiv.) in ACN (0.5 mL) and NMI (19.06 mg, 0.232 mmol, 3.50 equiv.), TCFH (27.9 mg, 0.099 mmol, 1.50 equiv.) was added dropwise at 0° C., and the mixture was stirred for 16 h at 50° C. The reaction mixture was concentrated in vacuo and the resulting mixture was extracted with EA (2×10 mL) and washed with H₂O (1×5 mL). The combined organic layers were washed with brine (1×10 mL), dried over anhydrous Na₂SO₄. After filtration, the filtrate was concentrated under reduced pressure. The crude product was purified by prep-TLC (PE:EA=1:5) to afford the title compound (3.2 mg, 11%) as a white solid. ESI-MS m/z=422.87 [M+H]⁺; Calculated MW: 422.06. ¹H NMR (400 MHz, Chloroform-d) δ 10.32 (s, 1H), 8.47 (s, 1H), 7.74 (d, J=1.3 Hz, 1H), 7.70-7.61 (m, 2H), 4.12 (s, 6H), 3.91 (s, 2H).

### Example 85: N—(4,4-dimethyl-7-(trifluoromethyl)-4,5-dihydrothiazolo[5,4-c]quinolin-2-yl)-4,6-dimethoxypyrimidine-5-carboxamide

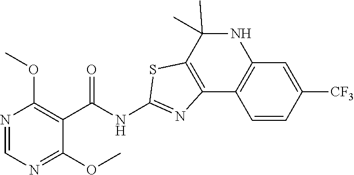 [SMILES: COc1ncnc(OC)c1C(=O)Nc1nc2c(s1)C(C)(C)Nc1cc(C(F)(F)F)ccc1-2]

### Example 85A: 4-(2-amino-4-(trifluoromethyl)phenyl)-2-methylbut-3-yn-2-ol

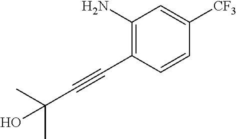 [SMILES: CC(C)(O)C#Cc1ccc(C(F)(F)F)cc1N]

Into a 50 mL round-bottom flask were added 2-bromo-5-(trifluoromethyl) aniline (1.00 g, 4.18 mmol, 1.00 equiv.), DMF (10.0 mL), 2-methylbut-3-yn-2-ol (422.0 mg, 5.02 mmol, 1.20 equiv.), CuI(40.0 mg, 0.209 mmol, 0.05 equiv.), Pd(PPh₃)₂Cl₂ (147.0 mg, 0.209 mmol, 0.05 equiv.) and TEA (1.27 g, 12.55 mmol, 3.00 equiv.) at room temperature. The resulting mixture was stirred for 5 h at 70° C. under argon atmosphere. The reaction was cooled down to room temperature and diluted with water. The resulting mixture was extracted with EtOAc (3×100 mL). The combined organic layers were washed with brine (2×80 mL), dried over anhydrous Na₂SO₄. After filtration, the filtrate was concentrated under reduced pressure. The residue was purified by silica gel column chromatography, eluted with PE/EtOAc (4:1) to afford the title compound (1.0 g, 98% yield) as a yellow solid. ESI-MS m/z=243.88 [M+H]⁺; Calculated MW: 243.09

### Example 85B: 2,2-dimethyl-7-(trifluoromethyl)-2,3-dihydroquinolin-4 (1H)-one

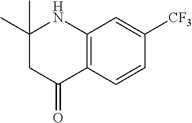 [SMILES: CC1(C)CC(=O)c2ccc(C(F)(F)F)cc2N1]

To a stirred solution of 4-(2-amino-4-(trifluoromethyl)phenyl)-2-methylbut-3-yn-2-ol (500.0 mg, 2.06 mmol, 1.00 equiv.) in H₂O (3.00 mL) was added HCl (con.) (3.00 mL) in portions at room temperature. The resulting mixture was stirred for 1.5 h at 120° C. under argon atmosphere. The reaction was cooled down to room temperature. The reaction was adjusted pH to 9 with NaHCO₃ (aq.). The resulting mixture was extracted with EtOAc (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EtOAc (4:1) to afford the title compound (300 mg, 60% yield) as a yellow oil. ESI-MS m/z=243.88 [M+H]⁺; Calculated MW: 243.09

### Example 85C: 4,4-dimethyl-7-(trifluoromethyl)-4,5-dihydrothiazolo[5,4-c]quinolin-2-amine

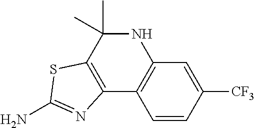 [SMILES: CC1(C)Nc2cc(C(F)(F)F)ccc2-c2nc(N)sc21]

To a stirred solution of 2,2-dimethyl-7-(trifluoromethyl)-2,3-dihydroquinolin-4 (1H)-one (50.0 mg, 0.206 mmol, 1.00 equiv.) in DMSO (2.0 mL) was added thiourea (16 mg, 0.206 mmol, 1.00 equiv.), I₂ (18 mg, 0.072 mmol, 0.35 equiv.), TsOH (78 mg, 0.412 mmol, 2.00 equiv.) in portions at room temperature. The resulting mixture was stirred for 16 h at 75° C. under air atmosphere. The reaction was cooled down to room temperature, then poured it into ice water. The resulting mixture was extracted with EtOAc (3×30 mL). The combined organic layers were washed with brine (2×20 mL), dried over anhydrous Na₂SO₄. After filtration, the filtrate was concentrated under reduced pressure. The residue was purified by prep-TLC (PE/EtOAc (2:1)) to afford the title compound (7 mg, 11% yield) as a yellow solid. ESI-MS m/z=299.88 [M+H]⁺; Calculated MW: 299.07.

### Example 85D: N—(4,4-dimethyl-7-(trifluoromethyl)-4,5-dihydrothiazolo[5,4-c]quinolin-2-yl)-4,6-dimethoxypyrimidine-5-carboxamide

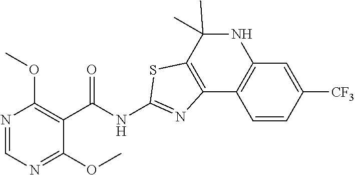 [SMILES: COc1ncnc(OC)c1C(=O)Nc1nc2c(s1)C(C)(C)Nc1cc(C(F)(F)F)ccc1-2]

To a stirred solution of 4,6-dimethoxypyrimidine-5-carboxylic acid (7 mg, 0.035 mmol, 1.50 equiv.) in DCM (1.00 mL) was added (COCl)₂ (15.0 mg, 0.117 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-dimethyl-7-(trifluoromethyl)-4,5-dihydrothiazolo[5,4-c]quinolin-2-amine (7.00 mg, 0.023 mmol, 1.00 equiv.) in THF (1.00 mL) was added KOH (7.00 mg, 0.117 mmol, 5.00 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (1.8 mg, 17% yield) as a light yellow solid. ESI-MS m/z=465.94 [M+H]⁺; Calculated MW: 465.11; ¹H NMR (400 MHz, Chloroform-d) δ 11.59 (s, 1H), 8.23 (s, 1H), 7.43 (d, J=7.9 Hz, 1H), 6.81 (d, J=7.9 Hz, 1H), 6.75 (s, 1H), 3.94 (s, 1H), 3.89 (s, 6H), 1.64 (s, 6H).

### Example 86: N—(8-chloro-4H-chromeno[4,3-d]thiazol-2-yl)-4,6-dimethoxypyrimidine-5-carboxamide

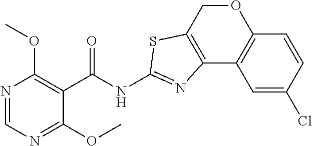 [SMILES: COc1ncnc(OC)c1C(=O)Nc1nc2c(s1)COc1ccc(Cl)cc1-2]

### Example 86A: 8-chloro-4H-chromeno[4,3-d]thiazol-2-amine

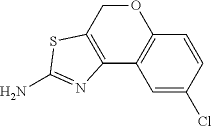 [SMILES: Nc1nc2c(s1)COc1ccc(Cl)cc1-2]

Into a 25 mL round-bottom flask were added 6-chlorochroman-4-one (500.0 mg, 2.747 mmol, 1.00 equiv.), hexane (10 mL), molecular sieve, pyrrolidine (975.00 mg, 13.73 mmol, 5.00 equiv.), and TsOH·H₂O (5 mg, 0.027 mmol, 0.01 equiv.) at room temperature. The resulting mixture was stirred for 3 h at 80° C. under argon atmosphere. The resulting mixture was cooled down to room temperature, then concentrated under reduced pressure. To the above mixture was added MeOH (5 mL), S (220 mg, 6.87 mmol, 2.50 equiv.) and NH₂CN (566 mg, 6.87 mmol, 2.50 equiv., 51% w.t. in H₂O) dropwise at 0° C. The resulting mixture was stirred for additional 16 h at room temperature. The reaction was concentrated under reduced pressure. The residue was diluted with water, extracted with EtOAc (3×50 mL). The combined organic layers were washed with brine (2×20 mL), dried over anhydrous Na₂SO₄. After filtration, the filtrate was concentrated under reduced pressure. The residue was purified by prep-TLC (PE/EtOAc (3:1)) to afford the title compound (120 mg, 18% yield) as a light yellow solid. ESI-MS m/z=238.77 [M+H]⁺; Calculated MW: 238.00.

### Example 86B: N—(8-chloro-4H-chromeno[4,3-d]thiazol-2-yl)-4,6-dimethoxypyrimidine-5-carboxamide

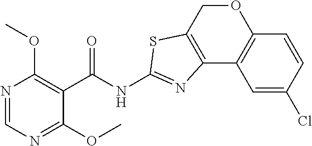 [SMILES: COc1ncnc(OC)c1C(=O)Nc1nc2c(s1)COc1ccc(Cl)cc1-2]

To a stirred solution of 4,6-dimethoxypyrimidine-5-carboxylic acid (46.0 mg, 0.252 mmol, 1.20 equiv.) in DCM (1.00 mL) was added (COCl)₂ (134.0 mg, 1.050 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 8-chloro-4H-chromeno[4,3-d]thiazol-2-amine (50.0 mg, 0.21 mmol, 1.00 equiv.) in THF (1.00 mL) was added KOH (35.0 mg, 0.63 mmol, 3.00 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (3.2 mg, 3.7% yield) as a light yellow solid. ESI-MS m/z=404.82 [M+H]⁺; Calculated MW: 404.03; ¹H NMR (400 MHz, Chloroform-d) δ 10.84 (s, 1H), 8.36 (s, 1H), 7.50 (d, J=2.6 Hz, 1H), 7.10 (dd, J=8.6, 2.6 Hz, 1H), 6.85 (d, J=8.6 Hz, 1H), 5.43 (s, 2H), 4.03 (s, 6H).

### Example 87: 4,6-dimethoxy-N—(4,4,5-trimethyl-7-(trifluoromethyl)-4,5-dihydrothiazolo[5,4-c]quinolin-2-yl)pyrimidine-5-carboxamide

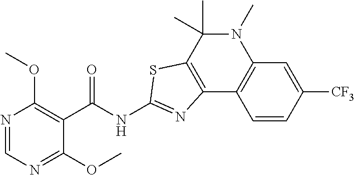 [SMILES: COc1ncnc(OC)c1C(=O)Nc1nc2c(s1)C(C)(C)N(C)c1cc(C(F)(F)F)ccc1-2]

### Example 87A: 1,2,2-trimethyl-7-(trifluoromethyl)-2,3-dihydroquinolin-4 (1H)-one

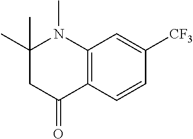 [SMILES: CN1c2cc(C(F)(F)F)ccc2C(=O)CC1(C)C]

To a stirred solution of 2,2-dimethyl-7-(trifluoromethyl)-2,3-dihydroquinolin-4 (1H)-one (250.0 mg, 1.03 mmol, 1.00 equiv.) in DMSO (4.0 mL) was added Cs₂CO₃ (1.012 g, 3.086 mmol, 3.00 equiv.) and Mel (1.46 g, 10.29 mmol, 10.00 equiv.) in portions at room temperature. The resulting mixture was stirred for 24 h at 100° C. The reaction was diluted with water, then extracted with EtOAc (3×80 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (4:1) to afford the title compound (200 mg, crude) as a yellow oil. ESI-MS m/z=257.90 [M+H]⁺; Calculated MW: 257.10

### Example 87B: 4,4,5-trimethyl-7-(trifluoromethyl)-4,5-dihydrothiazolo[5,4-c]quinolin-2-amine

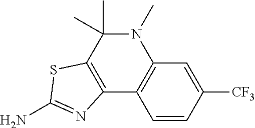 [SMILES: CN1c2cc(C(F)(F)F)ccc2-c2nc(N)sc2C1(C)C]

Into a 25 mL round-bottom flask were added 1,2,2-trimethyl-7-(trifluoromethyl)-2,3-dihydroquinolin-4 (1H)-one (200.0 mg, 0.778 mmol, 1.00 equiv.), hexane (5 mL), molecular sieve, pyrrolidine (276.0 mg, 3.88 mmol, 5.00 equiv.), and TsOH·H₂O (2 mg, 0.008 mmol, 0.01 equiv.) at room temperature. The resulting mixture was stirred for 3 h at 80° C. under argon atmosphere. The resulting mixture was cooled down to room temperature, then concentrated under reduced pressure. To the above mixture was added MeOH (2 mL), S (62 mg, 1.94 mmol, 2.50 equiv.) and NH₂CN (160 mg, 1.94 mmol, 2.50 equiv., 51% w.t. in H₂O) dropwise at 0° C. The resulting mixture was stirred for additional 16 h at 0° C. to room temperature. The reaction was concentrated under reduced pressure. The residue was diluted with water, extracted with EtOAc (3×50 mL). The combined organic layers were washed with brine (2×20 mL), dried over anhydrous Na₂SO₄. After filtration, the filtrate was concentrated under reduced pressure. The residue was purified by prep-TLC (PE/EtOAc (3:1)) to afford the title compound (20 mg, 8% yield) as a light yellow solid. ESI-MS m/z=313.91 [M+H]⁺; Calculated MW: 313.09.

### Example 87C: 4,6-dimethoxy-N—(4,4,5-trimethyl-7-(trifluoromethyl)-4,5-dihydrothiazolo[5,4-c]quinolin-2-yl)pyrimidine-5-carboxamide

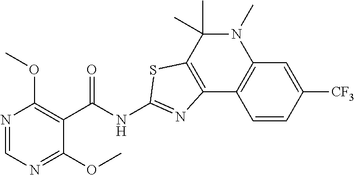 [SMILES: COc1ncnc(OC)c1C(=O)Nc1nc2c(s1)C(C)(C)N(C)c1cc(C(F)(F)F)ccc1-2]

To a stirred solution of 4,6-dimethoxypyrimidine-5-carboxylic acid (18.0 mg, 0.096 mmol, 1.50 equiv.) in DCM (1.00 mL) was added (COCl)₂ (41.0 mg, 0.320 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4,5-trimethyl-7-(trifluoromethyl)-4,5-dihydrothiazolo[5,4-c]quinolin-2-amine (20.0 mg, 0.064 mmol, 1.00 equiv.) in THF (1.00 mL) was added KOH (11.0 mg, 0.192 mmol, 3.00 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (11 mg, 36% yield) as an off-white solid. ESI-MS m/z=479.97 [M+H]⁺; Calculated MW: 479.12; ¹H NMR (400 MHz, Chloroform-d) δ 12.13 (s, 1H), 8.07 (s, 1H), 7.38 (d, J=7.7 Hz, 1H), 6.80 (dd, J=7.8, 1.5 Hz, 1H), 6.76 (d, J=1.6 Hz, 1H), 3.80 (s, 6H), 2.92 (s, 3H), 1.65 (s, 6H).

### Example 88: N—(5-allyl-4,4-dimethyl-7-(trifluoromethyl)-4,5-dihydrothiazolo[5,4-c]quinolin-2-yl)-4,6-dimethoxypyrimidine-5-carboxamide

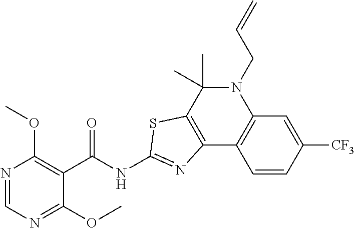 [SMILES: C=CCN1c2cc(C(F)(F)F)ccc2-c2nc(NC(=O)c3c(OC)ncnc3OC)sc2C1(C)C]

### Example 88A: 1-allyl-2,2-dimethyl-7-(trifluoromethyl)-2,3-dihydroquinolin-4 (1H)-one

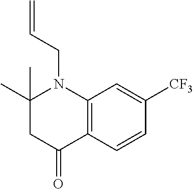 [SMILES: C=CCN1c2cc(C(F)(F)F)ccc2C(=O)CC1(C)C]

To a stirred solution of 2,2-dimethyl-7-(trifluoromethyl)-2,3-dihydroquinolin-4 (1H)-one (200.0 mg, 0.823 mmol, 1.00 equiv.) in DMSO (4.0 mL) was added Cs₂CO₃ (810.0 mg, 2.47 mmol, 3.00 equiv.), 3-bromoprop-1-ene (299.0 mg, 2.47 mmol, 3.00 equiv.) and in portions at room temperature. The resulting mixture was stirred for 24 h at 100° C. The reaction was diluted with water, then extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (4:1) to afford the title compound (165 mg, crude) as a yellow oil. ESI-MS m/z=283.93 [M+H]⁺; Calculated MW: 283.12.

### Example 88B: 5-allyl-4,4-dimethyl-7-(trifluoromethyl)-4,5-dihydrothiazolo[5,4-c]quinolin-2-amine

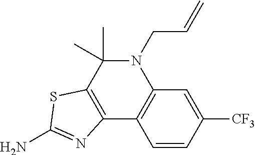 [SMILES: C=CCN1c2cc(C(F)(F)F)ccc2-c2nc(N)sc2C1(C)C]

Into a 25 mL round-bottom flask were added 1-allyl-2,2-dimethyl-7-(trifluoromethyl)-2,3-dihydroquinolin-4 (1H)-one (165.0 mg, 0.583 mmol, 1.00 equiv.), hexane (5 mL), molecular sieve, pyrrolidine (207.0 mg, 2.92 mmol, 5.00 equiv.), and TsOH·H₂O (2 mg, 0.006 mmol, 0.01 equiv.) at room temperature. The resulting mixture was stirred for 3 h at 80° C. under argon atmosphere. The resulting mixture was cooled down to room temperature, then concentrated under reduced pressure. To the above mixture was added MeOH (2 mL), S (47 mg, 1.46 mmol, 2.50 equiv.) and NH₂CN (120 mg, 1.46 mmol, 2.50 equiv., 51% w.t. in H₂O) dropwise at 0° C. The resulting mixture was stirred for additional 16 h at room temperature. The reaction was concentrated under reduced pressure. The residue was diluted with water, extracted with EtOAc (3×50 mL). The combined organic layers were washed with brine (2×20 mL), dried over anhydrous Na₂SO₄. After filtration, the filtrate was concentrated under reduced pressure. The residue was purified by prep-TLC (PE/EtOAc (3:1)) to afford the title compound (10 mg, 5% yield) as a light yellow solid. ESI-MS m/z=339.91 [M+H]⁺; Calculated MW: 339.10.

### Example 88C: N—(5-allyl-4,4-dimethyl-7-(trifluoromethyl)-4,5-dihydrothiazolo[5,4-c]quinolin-2-yl)-4,6-dimethoxypyrimidine-5-carboxamide

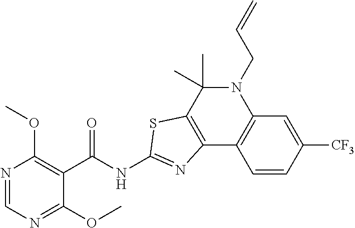 [SMILES: C=CCN1c2cc(C(F)(F)F)ccc2-c2nc(NC(=O)c3c(OC)ncnc3OC)sc2C1(C)C]

To a stirred solution of 4,6-dimethoxypyrimidine-5-carboxylic acid (8.0 mg, 0.044 mmol, 1.50 equiv.) in DCM (1.00 mL) was added (COCl)₂ (19.0 mg, 0.147 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 5-allyl-4,4-dimethyl-7-(trifluoromethyl)-4,5-dihydrothiazolo[5,4-c]quinolin-2-amine (10.00 mg, 0.029 mmol, 1.00 equiv.) in THF (1.00 mL) was added KOH (8.00 mg, 0.147 mmol, 5.00 equiv.) in portions at 0° C. The resulting mixture was stirred for 0.5 h at 0° C. under argon atmosphere. To the above mixture was added the acyl chloride/THE dropwise at 0° C. The resulting mixture was stirred for additional 16 h at 0° C. to room temperature. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (DCM/PE/EtOAc (7:4:1)) to afford the title compound (10 mg, 67% yield) as an off-white solid. ESI-MS m/z=505.95 [M+H]⁺; Calculated MW: 505.14; ¹H NMR (400 MHz, Chloroform-d) δ 12.28 (s, 1H), 8.04 (s, 1H), 7.36 (d, J=7.7 Hz, 1H), 6.72 (d, J=7.8 Hz, 1H), 6.67 (s, 1H), 5.93 (ddt, J=17.1, 9.3, 4.3 Hz, 1H), 5.40 (dd, J=17.1, 1.8 Hz, 1H), 5.27 (dd, J=10.6, 1.7 Hz, 1H), 4.03-3.97 (m, 2H), 3.80 (s, 6H), 1.68 (s, 6H).

### Example 89:3-chloro-N—(4,4-diethyl-7-(trifluoromethyl)-4H-chromeno[4,3-d]thiazol-2-yl)-6-methoxypyridazine-4-carboxamide

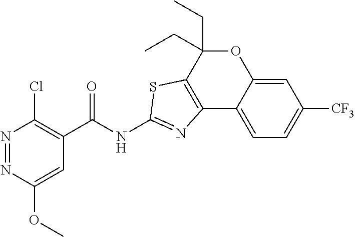 [SMILES: CCC1(CC)Oc2cc(C(F)(F)F)ccc2-c2nc(NC(=O)c3cc(OC)nnc3Cl)sc21]

### Example 89A: 3-chloro-6-methoxypyridazine-4-carboxylic acid

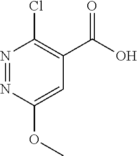

To a stirred solution of 3,6-dichloropyridazine-4-carboxylic acid (100 mg, 0.52 mmol, 1.0equiv.) in MeOH (2 mL) was added KOH (58.2 mg, 1.04 mmol, 2.0 equiv.) at 0° C. The reaction mixture was stirred for 2 hours at 70° C. Then the pH of the solution was adjusted to 4-5 by 4M HCl. Then the mixture was filtered, the filtrate was concentrated under vacuum to afford the title compound (40 mg, crude) as a white solid. ESI-MS m/z=188.78 [M+H]⁺; Calculated MW: 188.00

### Example 89B: 3-chloro-N—(4,4-diethyl-7-(trifluoromethyl)-4H-chromeno[4,3-d]thiazol-2-yl)-6-methoxypyridazine-4-carboxamide

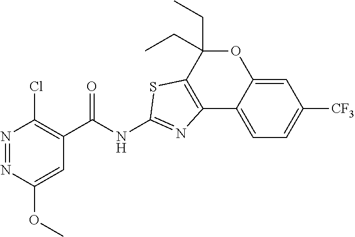 [SMILES: CCC1(CC)Oc2cc(C(F)(F)F)ccc2-c2nc(NC(=O)c3cc(OC)nnc3Cl)sc21]

To a stirred solution of 3-chloro-6-methoxypyridazine-4-carboxylic acid (26.3 mg, 0.14 mmol, 1.5 equiv.) in MeCN (0.5 mL) was added 4,4-diethyl-7-(trifluoromethyl)-4H-chromeno[4,3-d]thiazol-2-amine (30 mg, 0.09 mmol, 1.0 equiv.), TCFH (39.2 mg, 0.14 mmol, 1.5 equiv.) and NMI (26.2 mg, 0.32 mmol, 3.5 equiv.) at room temperature. The reaction mixture was stirred overnight at room temperature and concentrated and concentrated. The resulting residue was extracted with ethyl acetate (3×10 mL). The combined organic layers were washed with brine, dried over anhydrous Na₂SO₄. The resulting mixture was concentrated under vacuum. The crude product was purified by Prep-TLC (PE:EA=2:1) to afford the title compound (5.7 mg, 63.3%) as a yellow solid. ESI-MS m/z=498.90 [M+H]⁺; Calculated MW: 498.07; ¹H NMR (400 MHz, Chloroform-d) δ 11.48 (s, 1H), 7.44 (d, J=7.9 Hz, 1H), 7.23 (s, 1H), 7.11 (d, J=1.7 Hz, 1H), 7.07 (dd, J=7.9, 1.7 Hz, 1H), 4.05 (s, 3H), 2.05 (dq, J=14.7, 7.3 Hz, 2H), 1.92 (dq, J=14.6, 7.3 Hz, 2H), 1.01 (t, J=7.4 Hz, 6H).

### Example 90:6-chloro-N—(4,4-diethyl-7-(trifluoromethyl)-4H-chromeno[4,3-d]thiazol-2-yl)-3-methoxypyridazine-4-carboxamide

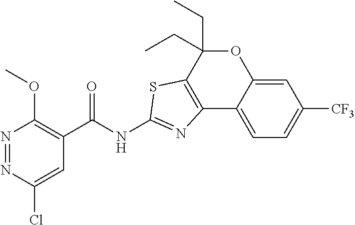 [SMILES: CCC1(CC)Oc2cc(C(F)(F)F)ccc2-c2nc(NC(=O)c3cc(Cl)nnc3OC)sc21]

### Example 90A: 6-chloro-3-methoxypyridazine-4-carboxylic acid

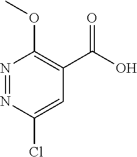

To a stirred solution of 3,6-dichloropyridazine-4-carboxylic acid (100 mg, 0.52 mmol, 1.0equiv.) in MeOH (2 mL) was added KOH (58.2 mg, 1.04 mmol, 2.0 equiv.) at 0° C. The reaction mixture was stirred for 2 hours at 70° C. Then the pH of the solution was adjusted to 4-5 by 4M HCl. Then the mixture was filtered, the filtrate was concentrated under vacuum to afford the title compound (40 mg, crude) as a white solid. ESI-MS m/z=188.78 [M+H]⁺; Calculated MW: 188.00

### Example 90B: 6-chloro-N—(4,4-diethyl-7-(trifluoromethyl)-4H-chromeno[4,3-d]thiazol-2-yl)-3-methoxypyridazine-4-carboxamide

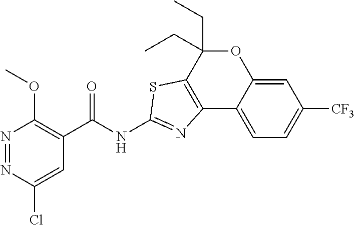 [SMILES: CCC1(CC)Oc2cc(C(F)(F)F)ccc2-c2nc(NC(=O)c3cc(Cl)nnc3OC)sc21]

To a stirred solution of 6-chloro-3-methoxypyridazine-4-carboxylic acid (26.3 mg, 0.14 mmol, 1.5 equiv.) in MeCN (0.5 mL) was added 4,4-diethyl-7-(trifluoromethyl)-4H-chromeno[4,3-d]thiazol-2-amine (30 mg, 0.09 mmol, 1.0 equiv.), TCFH (39.2 mg, 0.14 mmol, 1.5 equiv.) and NMI (26.2 mg, 0.32 mmol, 3.5 equiv.) at room temperature. The reaction mixture was stirred overnight at room temperature and concentrated. The resulting residue was extracted with ethyl acetate (3×10 mL). The combined organic layers were washed with brine, dried over anhydrous Na₂SO₄. The resulting mixture was concentrated under vacuum. The crude product was purified by Prep-TLC (PE:EA=2:1) to afford the title compound (3.0 mg, 6.7%) as a yellow solid. ESI-MS m/z=498.90 [M+H]⁺; Calculated MW: 498.07; H NMR (400 MHz, Chloroform-d) δ 10.88 (s, 1H), 8.25 (d, J=0.9 Hz, 1H), 7.77 (d, J=7.9 Hz, 1H), 7.20 (d, J=8.6 Hz, 1H), 7.14 (d, J=1.7 Hz, 1H), 4.50 (s, 3H), 2.09-1.98 (m, 2H), 1.91 (dq, J=14.6, 7.4 Hz, 2H), 0.98 (td, J=7.3, 4.5 Hz, 6H).

### Example 91: N—(4,4-diethyl-7-(trifluoromethyl)-4H-chromeno[4,3-d]thiazol-2-yl)-3,6-dimethoxypyridazine-4-carboxamide

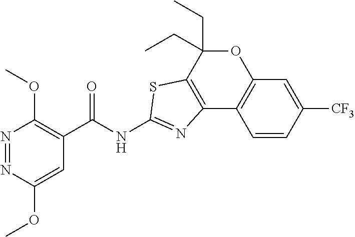 [SMILES: CCC1(CC)Oc2cc(C(F)(F)F)ccc2-c2nc(NC(=O)c3cc(OC)nnc3OC)sc21]

### Example 91A: 3,6-dimethoxypyridazine-4-carboxylic acid

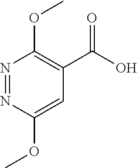

To MeOH (2.0 mL) was added KOH (233 mg, 4.16 mmol, 4 equiv.), 3,6-dichloropyridazine-4-carboxylic acid (200.0 mg, 1.04 mmol, 1 equiv.) in portions at 0° C. The resulting mixture was stirred for 16 h at 75° C. The reaction was adjusted pH to 5 with HCl/dioxane (4M). The resulting mixture was filtered, the filter cake was washed with EtOH (3×20 mL). After filtration, the filtrate was concentrated under reduced pressure. This resulted in the title compound (300 mg, crude) as a white solid. ESI-MS m/z=184.87 [M+H]⁺; Calculated MW: 184.05.

### Example 91B: N—(4,4-diethyl-7-(trifluoromethyl)-4H-chromeno[4,3-d]thiazol-2-yl)-3,6-dimethoxypyridazine-4-carboxamide

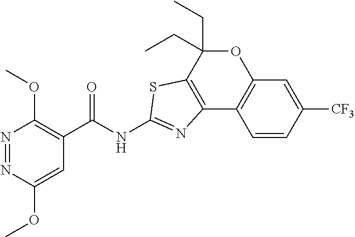 [SMILES: CCC1(CC)Oc2cc(C(F)(F)F)ccc2-c2nc(NC(=O)c3cc(OC)nnc3OC)sc21]

To a stirred solution of 3,6-dimethoxypyridazine-4-carboxylic acid (25.0 mg, 0.137 mmol, 1.50 equiv.) in DCM (1.00 mL) was added (COCl)₂ (58.0 mg, 0.457 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (30.0 mg, 0.091 mmol, 1.00 equiv.) in THF (1.00 mL) was added KOH (26.0 mg, 0.457 mmol, 5.00 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (DCM/PE/EtOAc (7:4:1)) to afford the title compound (20 mg, 44% yield) as a light yellow solid. ESI-MS m/z=494.95 [M+H]⁺; Calculated MW: 494.12; ¹H NMR (400 MHz, Chloroform-d) δ 11.02 (s, 1H), 7.82 (d, J=1.2 Hz, 1H), 7.78 (d, J=7.9 Hz, 1H), 7.22-7.17 (m, 1H), 7.13 (d, J=1.7 Hz, 1H), 4.40 (d, J=1.1 Hz, 3H), 4.13 (d, J=1.2 Hz, 3H), 2.09-1.97 (m, 2H), 1.96-1.84 (m, 2H), 0.96 (td, J=7.5, 1.3 Hz, 6H).

### Example 92: methyl 2′-(4,6-dimethoxypyrimidine-5-carboxamido)-7′-(trifluoromethyl)spiro[azetidine-3,4′-chromeno[4,3-d]thiazole]-1-carboxylate

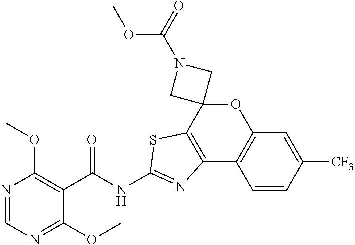 [SMILES: COC(=O)N1CC2(C1)Oc1cc(C(F)(F)F)ccc1-c1nc(NC(=O)c3c(OC)ncnc3OC)sc12]

### Example 92A: methyl pyridin-2-yl carbonate

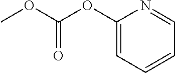

To a stirred solution of di(pyridin-2-yl) carbonate (100 mg, 0.46 mmol, 1.0equiv.) in THF (1 mL) was added MeONa (24.8 mg, 0.46 mmol, 1.0equiv.) at 0° C. The reaction mixture was stirred for 2 hours at room temperature. The resulting residue was extracted with ethyl acetate (3×5 mL). The combined organic layers were washed with saturated NH₄Cl solution, dried over anhydrous Na₂SO₄. Then combined organic layers were concentrated under vacuum to afford the title compound (20 mg, crude) as a white oil. ¹H NMR (400 MHz, Chloroform-d) δ 8.40 (ddt, J=4.2, 2.1, 1.0 Hz, 1H), 7.80 (ddt, J=7.9, 7.1, 1.9 Hz, 1H), 7.28-7.21 (m, 1H), 7.11 (dp, J=8.0, 0.8 Hz, 1H), 3.95-3.87 (m, 3H).

### Example 92B: methyl 2′-(4,6-dimethoxypyrimidine-5-carboxamido)-7′-(trifluoromethyl)spiro[azetidine-3,4′-chromeno[4,3-d]thiazole]-1-carboxylate

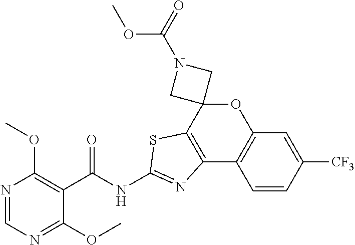 [SMILES: COC(=O)N1CC2(C1)Oc1cc(C(F)(F)F)ccc1-c1nc(NC(=O)c3c(OC)ncnc3OC)sc12]

To a stirred solution of 4,6-dimethoxy-N—(7′-(trifluoromethyl)spiro[azetidine-3,4′-chromeno[4,3-d]thiazol]-2′-yl)pyrimidine-5-carboxamide (20.0 mg, 0.041 mmol, 1.0 equiv.) in DCM (0.5 mL) was added methyl pyridin-2-yl carbonate (10.0 mg, 0.065 mmol, 1.5 equiv.), DIEA (8.1 mg, 0.065 mmol, 1.5 equiv.) at room temperature. The reaction mixture was stirred overnight at room temperature, diluted with DCM, washed with water, dried over anhydrous Na₂SO₄, and concentrated under vacuum. The crude product was purified by Prep-TLC (PE:EA=1:1) to afford the title compound (3.0 mg, 8.6%) as a white solid. ESI-MS m/z=537.88 [M+H]⁺; Calculated MW: 537.09. ¹H NMR (400 MHz, Chloroform-d) δ 11.05 (s, 1H), 8.33 (d, J=1.3 Hz, 1H), 7.61 (d, J=7.9 Hz, 1H), 7.22 (s, 1H), 7.19 (d, J=8.1 Hz, 1H), 4.50 (d, J=9.8 Hz, 2H), 4.36-4.29 (m, 2H), 4.01 (d, J=1.3 Hz, 6H), 3.74 (d, J=1.3 Hz, 3H).

### Example 93: N—(4,4-diethyl-7-(trifluoromethyl)-4H-chromeno[4,3-d]thiazol-2-yl)-3,5-dimethyl-1H-pyrazole-4-carboxamide

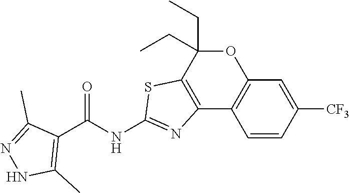 [SMILES: CCC1(CC)Oc2cc(C(F)(F)F)ccc2-c2nc(NC(=O)c3c(C)n[nH]c3C)sc21]

### Example 93A: ethyl 3,5-dimethyl-1-((2-(trimethylsilyl)ethoxy)methyl)-1H-pyrazole-4-carboxylate

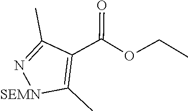 [SMILES: CCOC(=O)c1c(C)nn(COCC[Si](C)(C)C)c1C]

To a stirred solution of ethyl 3,5-dimethyl-1H-pyrazole-4-carboxylate (100.0 mg, 0.595 mmol, 1.00 equiv.) in THF (2.00 mL) was added NaH (39.0 mg, 0.893 mmol, 1.50 equiv.) in portions at 0° C. The resulting mixture was stirred for 0.5 h at 0° C. under argon. To the above mixture was added SEMCl (118.0 mg, 0.655 mmol, 1.10 equiv.) at 0° C. The resulting mixture was stirred for additional 2 h at room temperature. The reaction was quenched with sat. NH₄Cl(aq.) at 0° C. The resulting mixture was extracted with EtOAc (3×20 mL). The combined organic layers were washed with brine (2×10 mL) and dried over anhydrous Na₂SO₄. After filtration, the filtrate was concentrated under reduced pressure. The residue was purified by prep-TLC (PE/EtOAc (4:1)) to afford ethyl 3,5-dimethyl-1-((2-(trimethylsilyl)ethoxy)methyl)-1H-pyrazole-4-carboxylate (145.0 mg, 81% yield) as a colorless oil. ESI-MS m/z=299.07 [M+H]⁺; Calculated MW: 298.17.

### Example 93B: 3,5-dimethyl-1-((2-(trimethylsilyl)ethoxy)methyl)-1H-pyrazole-4-carboxylic acid

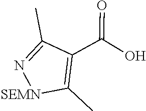 [SMILES: Cc1nn(COCC[Si](C)(C)C)c(C)c1C(=O)O]

To a stirred solution of ethyl 3,5-dimethyl-1-((2-(trimethylsilyl)ethoxy)methyl)-1H-pyrazole-4-carboxylate (50.0 mg, 0.167 mmol, 1.00 equiv.) in MeOH (1.00 mL) was added KOH (28.0 mg, 0.503 mmol, 3.00 equiv.) in H₂O (0.5 mL) in portions at 0° C. The resulting mixture was stirred for 6 h at 80° C. under argon. The reaction was adjusted pH to 5 with HCl (1 M). The resulting mixture was extracted with DCM (3×20 mL). The combined organic layers were washed with brine (2×10 mL), dried over anhydrous Na₂SO₄. After filtration, the filtrate was concentrated under reduced pressure. This resulted in 3,5-dimethyl-1-((2-(trimethylsilyl)ethoxy)methyl)-1H-pyrazole-4-carboxylic acid (35.0 mg, crude) as a white solid. ESI-MS m/z=271.05 [M+H]⁺; Calculated MW: 270.14

### Example 93C: N—(4,4-diethyl-7-(trifluoromethyl)-4H-chromeno[4,3-d]thiazol-2-yl)-3,5-dimethyl-1-((2-(trimethylsilyl)ethoxy)methyl)-1H-pyrazole-4-carboxamide

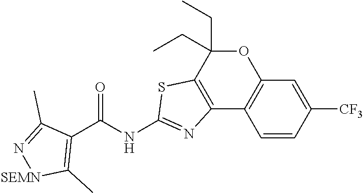 [SMILES: CCC1(CC)Oc2cc(C(F)(F)F)ccc2-c2nc(NC(=O)c3c(C)nn(COCC[Si](C)(C)C)c3C)sc21]

To a stirred solution of 4,4-diethyl-7-(trifluoromethyl)-4H-chromeno[4,3-d]thiazol-2-amine (42.0 mg, 0.129 mmol, 1.00 equiv.) and 3,5-dimethyl-1-((2-(trimethylsilyl)ethoxy)methyl)-1H-pyrazole-4-carboxylic acid (35.0 mg, 0.129 mmol, 1.00 equiv.) in ACN (2.00 mL) was added NMI (37.0 mg, 0.451 mmol, 3.50 equiv.) and TCFH (54.0 mg, 0.193 mmol, 1.50 equiv.) in portions at 0° C. The resulting mixture was stirred for 16 h at 60° C. under argon atmosphere. The reaction was diluted with water, extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford N—(4,4-diethyl-7-(trifluoromethyl)-4H-chromeno[4,3-d]thiazol-2-yl)-3,5-dimethyl-1-((2-(trimethylsilyl)ethoxy)methyl)-1H-pyrazole-4-carboxamide (23.0 mg, 30% yield) as a light yellow oil. ESI-MS m/z=581.21 [M+H]⁺; Calculated MW: 580.22

### Example 93D: N—(4,4-diethyl-7-(trifluoromethyl)-4H-chromeno[4,3-d]thiazol-2-yl)-3,5-dimethyl-1H-pyrazole-4-carboxamide

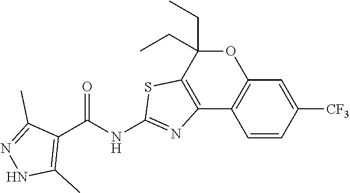 [SMILES: CCC1(CC)Oc2cc(C(F)(F)F)ccc2-c2nc(NC(=O)c3c(C)n[nH]c3C)sc21]

To a stirred solution of N—(4,4-diethyl-7-(trifluoromethyl)-4H-chromeno[4,3-d]thiazol-2-yl)-3,5-dimethyl-1-((2-(trimethylsilyl)ethoxy)methyl)-1H-pyrazole-4-carboxamide (23.0 mg, 0.039 mmol, 1.00 equiv.) in DCM (1.00 mL) was added TFA (0.25 mL) dropwise at 0° C. The resulting mixture was stirred for 16 h at room temperature under argon atmosphere. The resulting mixture was concentrated under reduced pressure. The residue was adjusted pH to 9 with NaHCO₃ (aq.), extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (1:1)) to afford N—(4,4-diethyl-7-(trifluoromethyl)-4H-chromeno[4,3-d]thiazol-2-yl)-3,5-dimethyl-1H-pyrazole-4-carboxamide (10.0 mg, 56% yield) as a white solid. ESI-MS m/z=451.11 [M+H]⁺; Calculated MW: 450.13; ¹H NMR (400 MHz, Chloroform-d) δ 7.71 (d, J=7.9 Hz, 1H), 7.17 (d, J=8.0 Hz, 1H), 7.12 (s, 1H), 2.63 (s, 6H), 2.02 (dq, J=14.7, 7.4 Hz, 2H), 1.89 (dq, J=14.6, 7.3 Hz, 2H), 0.97 (t, J=7.4 Hz, 6H).

### Example 94: N—(4,4-diethyl-7-(trifluoromethyl)-4H-chromeno[4,3-d]thiazol-2-yl)-3-methoxy-1H-pyrazole-4-carboxamide

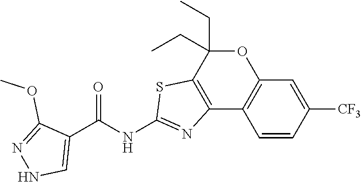 [SMILES: CCC1(CC)Oc2cc(C(F)(F)F)ccc2-c2nc(NC(=O)c3c[nH]nc3OC)sc21]

### Example 94A: methyl 3-methoxy-1H-pyrazole-4-carboxylate

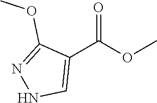

To a stirred solution of dimethyl 2-(methoxymethylene) malonate (1.00 g, 5.74 mmol, 1.00 equiv.) in MeOH (20.00 mL) was added NH₂NH₂·2HCl (1.2 g, 11.49 mmol, 2.00 equiv.) in portions at room temperature. The resulting mixture was stirred for 16 h at 70° C. under argon atmosphere. The resulting mixture was concentrated under reduced pressure, adjusted pH to 9 with NaHCO₃ (aq.) and extracted with EtOAc (3×80 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (1:1) to afford methyl 3-methoxy-1H-pyrazole-4-carboxylate (210.0 mg, 23% yield) as a yellow oil. ¹H NMR (400 MHz, Chloroform-d) δ 7.89 (s, 1H), 4.00 (s, 3H), 3.82 (s, 3H).

### Example 94B: methyl 3-methoxy-1-(4-methoxybenzyl)-1H-pyrazole-4-carboxylate

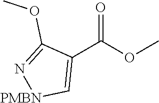

To a stirred solution of methyl 3-methoxy-1H-pyrazole-4-carboxylate (100.0 mg, 0.641 mmol, 1.00 equiv.) and K₂CO₃ (133.0 mg, 0.961 mmol, 1.50 equiv.) in ACN (3.00 mL) was added PMBCl (110.0 mg, 0.705 mmol, 1.10 equiv.) in portions at room temperature. The resulting mixture was stirred for 16 h at 80° C. under argon atmosphere. The reaction was diluted with water, extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (10:1)) to afford methyl 3-methoxy-1-(4-methoxybenzyl)-1H-pyrazole-4-carboxylate (175.0 mg, 98% yield) as a colorless oil. ESI-MS m/z=277.03 [M+H]⁺; Calculated MW: 276.11.

### Example 94C: 3-methoxy-1-(4-methoxybenzyl)-1H-pyrazole-4-carboxylic acid

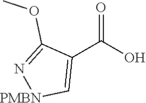

To a stirred solution of methyl 3-methoxy-1-(4-methoxybenzyl)-1H-pyrazole-4-carboxylate (50.0 mg, 0.181 mmol, 1.00 equiv.) in MeOH (1.0 mL) was added KOH (50.0 mg, 0.905 mmol, 5.00 equiv.) in H₂O (0.5 mL) in portions at 0° C. The resulting mixture was stirred for 3 h at 70° C. under argon atmosphere. The reaction was adjusted pH to 5 with HCl (1 M). The resulting mixture was extracted with DCM (3×20 mL). The combined organic layers were washed with brine (2×10 mL), dried over anhydrous Na₂SO₄. After filtration, the filtrate was concentrated under reduced pressure. This resulted in 3-methoxy-1-(4-methoxybenzyl)-1H-pyrazole-4-carboxylic acid (34.0 mg, crude) as a white solid. ESI-MS m/z=263.00 [M+H]⁺; Calculated MW: 262.10.

### Example 94D: N—(4,4-diethyl-7-(trifluoromethyl)-4H-chromeno[4,3-d]thiazol-2-yl)-3-methoxy-1-(4-methoxybenzyl)-1H-pyrazole-4-carboxamide

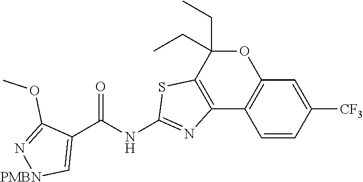

To a stirred solution of 3-methoxy-1-(4-methoxybenzyl)-1H-pyrazole-4-carboxylic acid (34.0 mg, 0.13 mmol, 1.00 equiv.) and 4,4-diethyl-7-(trifluoromethyl)-4H-chromeno[4,3-d]thiazol-2-amine (43.0 mg, 0.13 mmol, 1.00 equiv.) in ACN (5.00 mL) was added TCFH (54.0 mg, 0.195 mmol, 1.50 equiv.) and NMI (37.0 mg, 0.455 mmol, 3.50 equiv.) at 0° C. The resulting mixture was stirred for 16 h at 70° C. under argon atmosphere. The resulting mixture was filtered, the filter cake was washed with ACN (3×3 mL). The filtrate was concentrated under reduced pressure. This resulted in N—(4,4-diethyl-7-(trifluoromethyl)-4H-chromeno[4,3-d]thiazol-2-yl)-3-methoxy-1-(4-methoxybenzyl)-1H-pyrazole-4-carboxamide (35.0 mg, crude) as a white solid. ESI-MS m/z=573.17 [M+H]⁺; Calculated MW: 572.17.

### Example 94E: N—(4,4-diethyl-7-(trifluoromethyl)-4H-chromeno[4,3-d]thiazol-2-yl)-3-methoxy-1H-pyrazole-4-carboxamide

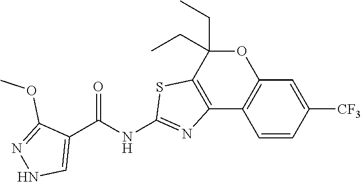 [SMILES: CCC1(CC)Oc2cc(C(F)(F)F)ccc2-c2nc(NC(=O)c3c[nH]nc3OC)sc21]

A mixture of N—(4,4-diethyl-7-(trifluoromethyl)-4H-chromeno[4,3-d]thiazol-2-yl)-3-methoxy-1-(4-methoxybenzyl)-1H-pyrazole-4-carboxamide (25 mg, 0.43 mmol, 1.00 equiv.) in TFA (5.00 mL) was stirred for 6 h at 70° C. under argon atmosphere. After the reaction was completed, the reaction was cooled to room temperature and TFA was removed by concentration under reduced pressure. The residue was adjusted pH to 8 with sat. NaHCO₃ (aq.) The resulting mixture was extracted with EtOAc (3×10 mL). The combined organic layers were dried over anhydrous Na₂SO₄. After filtration, the filtrate was concentrated under reduced pressure. The residue was purified by prep-TLC (PE/EtOAc (1:1)) to afford N—(4,4-diethyl-7-(trifluoromethyl)-4H-chromeno[4,3-d]thiazol-2-yl)-3-methoxy-1H-pyrazole-4-carboxamide (11.0 mg, 56% yield) as a white solid. ESI-MS m/z=453.09 [M+H]⁺; Calculated MW: 452.11; ¹H NMR (400 MHz, Chloroform-d) δ 9.93 (s, 1H), 8.09 (s, 1H), 7.76 (d, J=7.9 Hz, 1H), 7.18 (d, J=7.9 Hz, 1H), 7.11 (d, J=1.7 Hz, 1H), 4.18 (s, 3H), 2.01 (dq, J=14.7, 7.4 Hz, 2H), 1.89 (dq, J=14.7, 7.4 Hz, 2H), 0.96 (t, J=7.4 Hz, 6H).

### Example 95: 4,6-dimethoxy-N-(1′-oxido-7-(trifluoromethyl)-2′,3′,5′,6′-tetrahydrospiro[chromeno[3,4-d]thiazole-4,4′-thiopyran]-2-yl)pyrimidine-5-carboxamide

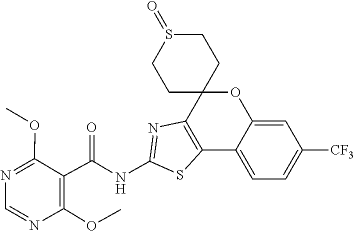 [SMILES: COc1ncnc(OC)c1C(=O)Nc1nc2c(s1)-c1ccc(C(F)(F)F)cc1OC21CCS(=O)CC1]

### Example 95A: 7-(trifluoromethyl)-2′,3′,5′,6′-tetrahydrospiro[chromane-2,4′-thiopyran]-4-one

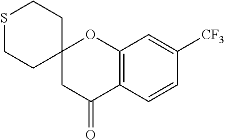 [SMILES: O=C1CC2(CCSCC2)Oc2cc(C(F)(F)F)ccc21]

To a stirred solution of 2-ethyl-5-(trifluoromethyl) phenol (500 mg, 2.45 mmol, 1.00 equiv.) in MeOH (5.00 mL) was added tetrahydro-4H-thiopyran-4-one (341 mg, 2.94 mmol, 1.20 equiv.) and pyrrolidine (348.0 mg, 4.90 mmol, 2.00 equiv.). The resulting mixture was stirred for 16 h at room temperature under argon atmosphere. After the reaction was completed, MeOH was removed by concentration under reduced pressure, the residue was diluted with H₂O, extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EtOAc (6:1) to afford 7-(trifluoromethyl)-2′,3′,5′,6′-tetrahydrospiro[chromane-2,4′-thiopyran]-4-one (576 mg, 78% yield) as a white solid. ¹H NMR (400 MHz, Chloroform-d) δ 7.95 (d, J=8.1 Hz, 1H), 7.29 (s, 1H), 7.23 (d, J=7.9 Hz, 1H), 3.05 (ddd, J=14.3, 11.9, 2.5 Hz, 2H), 2.73 (s, 2H), 2.50-2.28 (m, 4H), 1.83 (ddd, J=14.8, 12.1, 3.6 Hz, 2H).

### Example 95B: 7-(trifluoromethyl)-2′,3′,5′,6′-tetrahydrospiro[chromeno[4,3-d]thiazole-4,4′-thiopyran]-2-amine

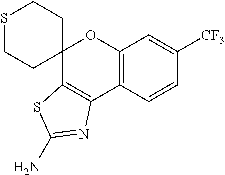 [SMILES: Nc1nc2c(s1)C1(CCSCC1)Oc1cc(C(F)(F)F)ccc1-2]

To a stirred solution of 7-(trifluoromethyl)-2′,3′,5′,6′-tetrahydrospiro[chromane-2,4′-thiopyran]-4-one (528.0 mg, 1.85 mmol, 1.00 equiv.) in Toluene (10.00 mL) was added p-TsOH (18 mg, 0.093 mmol, 0.05 equiv.) and pyrrolidine (655.0 mg, 9.23 mmol, 5.00 equiv.). The resulting mixture was stirred for 4 h at 130° C. under argon atmosphere. The reaction was cooled down to room temperature and toluene was removed by concentration under reduced pressure, the residue was dispersed in MeOH. To the mixture was added sulfur (148.00 mg, 4.62 mmol, 2.50 equiv.) in portions at 0° C. The resulting mixture was stirred for 1 h at 0° C. Then to the above mixture was added NH₂—CN (194 mg, 4.62 mmol, 2.50 equiv., 51% w.t.% in water). The resulting mixture was stirred for 14 h at room temperature. After the reaction was completed, MeOH was removed by concentration under reduced pressure, the residue was diluted with water, extracted with EtOAc (3×50 mL). The combined organic layers were dried over anhydrous Na₂SO₄. After filtration, the filtrate was concentrated under reduced pressure. The residue was purified by silica gel column chromatography, eluted with PE/EtOAc (4:1) to afford 7-(trifluoromethyl)-2′,3′,5′,6′-tetrahydrospiro[chromeno[4,3-d]thiazole-4,4′thiopyran]-2-amine (166.0 mg, 25% yield) as a yellow solid. ¹H NMR (400 MHz, Chloroform-d) δ 7.68-7.62 (m, 1H), 7.22 (ddd, J=7.8, 1.7, 0.8 Hz, 1H), 7.19 (d, J=1.8 Hz, 1H), 5.06 (s, 2H), 3.26-3.15 (m, 2H), 2.48 (t, J=17.5 Hz, 4H), 1.94 (td, J=13.1, 12.6, 3.3 Hz, 2H).

### Example 95C: 4,6-dimethoxy-N—(7-(trifluoromethyl)-2′,3′,5′,6′-tetrahydrospiro[chromeno[4,3-d]thiazole-4,4′-thiopyran]-2-yl)pyrimidine-5-carboxamide

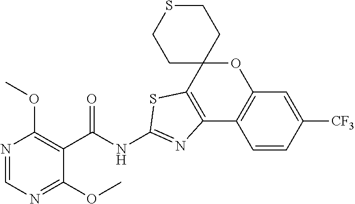 [SMILES: COc1ncnc(OC)c1C(=O)Nc1nc2c(s1)C1(CCSCC1)Oc1cc(C(F)(F)F)ccc1-2]

To a stirred solution of 4,6-dimethoxypyrimidine-5-carboxylic acid (67.0 mg, 0.36 mmol, 1.30 equiv.) in DCM (5.00 mL) was added (COCl)₂ (177.0 mg, 1.40 mmol, 5.00 equiv.) and DMF (1.00 mg). The resulting mixture was stirred for 2 h at room temperature under argon atmosphere. After the reaction was completed, DCM was removed by concentration under reduced pressure, the residue was dispersed in THF (2.00 mL). To a stirred solution of 7-(trifluoromethyl)-2′,3′,5′,6′-tetrahydrospiro[chromeno[4,3-d]thiazole-4,4′thiopyran]-2-amine (100.0 mg, 0.28 mmol, 1.00 equiv.) in THF (5.00 mL) was added KOH (28.00 mg, 1.40 mmol, 5.00 equiv.) and at 0° C. The resulting mixture was stirred for 0.5 h at room temperature under argon atmosphere. Then the acyl chloride solution was added. The resulting mixture was stirred for 16 h at room temperature. After the reaction was completed, the reaction was quenched with sat. NH₄Cl(aq.), extracted with EtOAc (3×20 mL). The combined organic layers were dried over anhydrous Na₂SO₄. After filtration, the filtrate was concentrated under reduced pressure. The residue was purified by prep-TLC (PE/EtOAc/DCM (4:1:1)) to afford (50.0 mg, 34% yield) as a white solid. ¹H NMR (400 MHz, Chloroform-d) δ 11.00 (s, 1H), 8.33 (s, 1H), 7.60 (d, J=7.9 Hz, 1H), 7.24-7.21 (m, 1H), 7.16 (d, J=8.1 Hz, 1H), 3.99 (s, 6H), 3.32-3.16 (m, 2H), 2.61-2.45 (m, 4H), 2.12-2.02 (m, 2H).

### Example 95D: 4,6-dimethoxy-N-(l′-oxido-7-(trifluoromethyl)-2′,3′,5′,6′-tetrahydrospiro[chromeno[3,4-d]thiazole-4,4′-thiopyran]-2-yl)pyrimidine-5-carboxamide

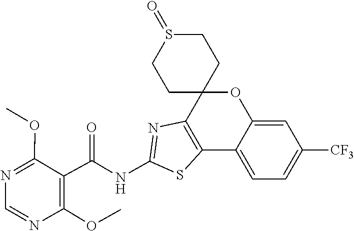 [SMILES: COc1ncnc(OC)c1C(=O)Nc1nc2c(s1)-c1ccc(C(F)(F)F)cc1OC21CCS(=O)CC1]

To a stirred solution of 4,6-dimethoxy-N—(7-(trifluoromethyl)-2′,3′,5′,6′-tetrahydrospiro[chromeno[4,3-d]thiazole-4,4′-thiopyran]-2-yl)pyrimidine-5-carboxamide (25.0 mg, 0.048 mmol, 1.00 equiv.) in DCM (1.0 mL) was added m-CPBA (7.4 mg, 0.043 mmol, 0.90 equiv.) in portions at 0° C. The resulting mixture was stirred for 16 h at room temperature under argon atmosphere. The reaction was diluted with DCM (10.0 mL) washed with NaHCO₃ (2×10 mL), brine (2×10 mL), dried over anhydrous Na₂SO₄. After filtration, the filtrate was concentrated under reduced pressure. The residue was purified by prep-TLC (PE/EtOAc (1:4)) to afford 4,6-dimethoxy-N—(1′-oxido-7-(trifluoromethyl)-2′,3′,5′,6′-tetrahydrospiro[chromeno[4,3-d]thiazole-4,4′-thiopyran]-2-yl)pyrimidine-5-carboxamide (9.0 mg, 34% yield) as a white solid. ESI-MS m/z=541.07 [M+H]⁺; Calculated MW: 540.07; ¹H NMR (400 MHz, Chloroform-d) δ 10.89 (s, 1H), 8.36 (s, 1H), 7.67 (d, J=8.2 Hz, 1H), 7.30-7.26 (m, 1H), 7.18 (d, J=1.7 Hz, 1H), 4.01 (s, 6H), 3.37-3.19 (m, 2H), 3.10-2.93 (m, 2H), 2.86-2.72 (m, 1H), 2.65 (s, 1H), 2.31 (d, J=14.2 Hz, 2H).

### Example 96: N—(4,4-diethyl-7-(trifluoromethyl)-4H-chromeno[4,3-d]thiazol-2-yl)-3-methoxypyrazine-2-carboxamide

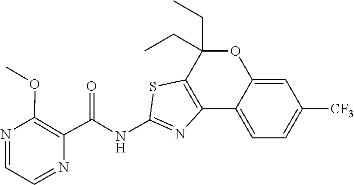 [SMILES: CCC1(CC)Oc2cc(C(F)(F)F)ccc2-c2nc(NC(=O)c3nccnc3OC)sc21]

To a stirred solution of 3-methoxypyrazine-2-carboxylic acid (14 mg, 0.09 mmol, 1.0equiv.) in MeCN (0.5 mL) was added 4,4-diethyl-7-(trifluoromethyl)-4H-chromeno[4,3-d]thiazol-2-amine (30 mg, 0.09 mmol, 1.0equiv.), TCFH (39.2 mg, 0.14 mmol, 1.5equiv.) and NMI (26.2 mg, 0.32 mmol, 3.5equiv.) at room temperature. The reaction mixture was stirred overnight at room temperature and concentrated. The resulting residue was extracted with ethyl acetate (3×10 mL). The combined organic layers were washed with brine, dried over anhydrous Na₂SO₄. After concentration, the crude product was purified by Prep-TLC (PE:EA=2:1) to afford the title compound (6.9 mg, 14%) as a yellow solid. ESI-MS m/z=464.94 [M+H]⁺; Calculated MW: 464.11. ¹H NMR (400 MHz, Chloroform-d) δ 11.00 (s, 1H), 8.43 (d, J=2.0 Hz, 1H), 8.26 (t, J=2.1 Hz, 1H), 7.73 (d, J=7.8 Hz, 1H), 7.18 (d, J=7.9 Hz, 1H), 7.12 (s, 1H), 4.18 (d, J=1.6 Hz, 3H), 2.02 (ddd, J=13.5, 8.4, 6.8 Hz, 2H), 1.96-1.85 (m, 2H), 0.97 (td, J=7.4, 1.7 Hz, 6H).

### Example 97: N—(4,4-diethyl-7-(trifluoromethyl)-4H-chromeno[4,3-d]thiazol-2-yl)-4,6-bis(methoxy-d3)pyrimidine-5-carboxamide

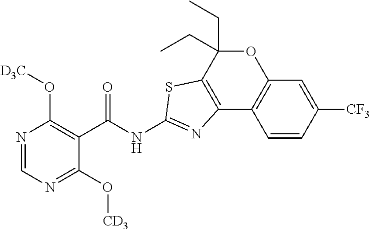 [SMILES: [2H]C([2H])([2H])Oc1ncnc(OC([2H])([2H])[2H])c1C(=O)Nc1nc2c(s1)C(CC)(CC)Oc1cc(C(F)(F)F)ccc1-2]

### Example 97A: 4,6-bis(methoxy-d3)pyrimidine-5-carboxylic acid

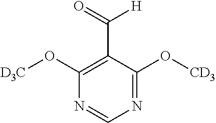 [SMILES: [2H]C([2H])([2H])Oc1ncnc(OC([2H])([2H])[2H])c1C=O]

To a stirred solution of CD₃OD (2.00 mL) was added NaH (84.0 mg, 2.08 mmol, 4.00 equiv.) in portions at 0° C. The resulting mixture was stirred for 0.5 h at 0° C. under argon atmosphere. To the above mixture was added 4,6-dichloropyrimidine-5-carboxylic acid (100.0 mg, 0.521 mmol, 1.00 equiv.) in CD₃OD (0.5 mL) dropwise at 0° C. The resulting mixture was stirred for additional 16 h at 60° C. The reaction was diluted with water (20 mL), adjusted pH to 5 with 1 M HCl(aq.). The resulting mixture was filtered, the filter cake was washed with EA (3×20 mL), dried in the oven. This resulted in the title compound (72 mg, crude) as an off-white solid. ESI-MS m/z=191.03 [M+H]⁺; Calculated MW: 190.09.

### Example 97B: N—(4,4-diethyl-7-(trifluoromethyl)-4H-chromeno[4,3-d]thiazol-2-yl)-4,6-bis(methoxy-d3)pyrimidine-5-carboxamide

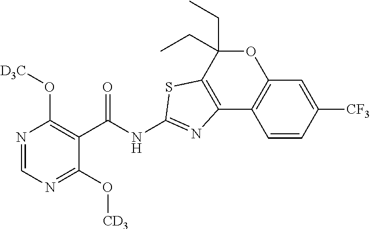 [SMILES: [2H]C([2H])([2H])Oc1ncnc(OC([2H])([2H])[2H])c1C(=O)Nc1nc2c(s1)C(CC)(CC)Oc1cc(C(F)(F)F)ccc1-2]

To a stirred solution of 4,6-bis(methoxy-d3)pyrimidine-5-carboxylic acid (26.0 mg, 0.137 mmol, 1.50 equiv.) in DCM (1.00 mL) was added (COCl)₂ (58.0 mg, 0.457 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (30.0 mg, 0.091 mmol, 1.00 equiv.) in THF (1.00 mL) was added KOH (26.0 mg, 0.192 mmol, 5.00 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (3:1)) to afford the title compound (35 mg, 76% yield) as an off-white solid. ESI-MS m/z=500.99 [M+H]⁺; Calculated MW: 500.16; ¹H NMR (400 MHz, Chloroform-d) δ 11.75 (s, 1H), 8.19 (t, J=1.2 Hz, 1H), 7.43 (d, J=7.9 Hz, 1H), 7.09 (d, J=2.2 Hz, 1H), 7.01 (d, J=7.9 Hz, 1H), 2.02 (ddd, J=14.5, 7.4, 1.7 Hz, 2H), 1.90 (ddd, J=14.5, 7.3, 1.7 Hz, 2H), 1.00 (td, J=7.4, 1.7 Hz, 6H).

### Example 98: N—(4,4-diethyl-7-(trifluoromethyl)-4H-chromeno[4,3-d]thiazol-2-yl)-2,4-dimethoxynicotinamide

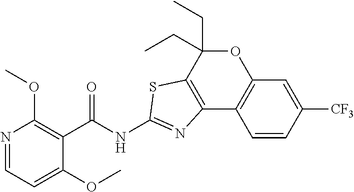 [SMILES: CCC1(CC)Oc2cc(C(F)(F)F)ccc2-c2nc(NC(=O)c3c(OC)ccnc3OC)sc21]

To a stirred solution of 2,4-dimethoxynicotinic acid (25.0 mg, 0.137 mmol, 1.50 equiv.) in DCM (1.00 mL) was added (COCl)₂ (58.0 mg, 0.457 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of 4,4-diethyl-7-(trifluoromethyl)-4H-chromeno[4,3-d]thiazol-2-amine (30.0 mg, 0.091 mmol, 1.00 equiv.) in THF (1.00 mL) was added KOH (26.00 mg, 0.192 mmol, 5.00 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (DCM/PE/EtOAc (7:4:1)) to afford the title compound (13 mg, 28% yield) as a light yellow solid. ESI-MS m/z=494.00 [M+H]⁺; Calculated MW: 493.13; ¹H NMR (400 MHz, Chloroform-d) δ 11.77 (s, 1H), 7.83 (dd, J=6.3, 2.5 Hz, 1H), 7.44-7.35 (m, 1H), 7.07 (d, J=2.2 Hz, 1H), 6.98 (d, J=7.8 Hz, 1H), 6.21 (dd, J=6.3, 2.5 Hz, 1H), 3.77 (dd, J=8.3, 2.5 Hz, 6H), 1.95 (dddd, J=43.9, 14.4, 7.2, 2.5 Hz, 4H), 1.01 (td, J=7.5, 2.5 Hz, 6H).

### Example 99: 4,6-dimethoxy-N—(9-(trifluoromethyl)-4H-chromeno[4,3-d]thiazol-2-yl)pyrimidine-5-carboxamide

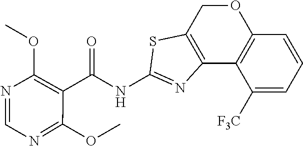 [SMILES: COc1ncnc(OC)c1C(=O)Nc1nc2c(s1)COc1cccc(C(F)(F)F)c1-2]

### Example 99A: 3-(3-(trifluoromethyl)phenoxy)propanoic acid

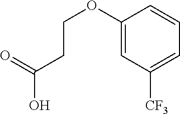 [SMILES: O=C(O)CCOc1cccc(C(F)(F)F)c1]

3-(trifluoromethyl) phenol (1 g, 6.17 mmol, 1 equiv.) and 3-chloropropanoic acid (1.34 g, 12.34 mmol, 2 equiv.) were added to a flame-dried three necked flask. NaOH solution (2 mmol/mL, 8 mL, 16.0 mmol, 2.6 equiv.) was added to the mixture. The mixture was stirred at reflux for 3 hours. The mixture was cooled to room temperature and acidified with 6N HCl to pH=5~6. The aqueous was extracted with EA (20 mL×2). The combine organic layers were washed with brine (20 mL×2), dried over Na₂SO₄, filtrated, and concentrated under reduce pressure. The crude product was purified by silica gel column chromatography, eluted with PE:EA=20:1 to afford the title compound (200 mg, 14%) as yellow solid. ¹H NMR (400 MHz, Chloroform-d) δ 7.38 (t, J=8.0 Hz, 1H), 7.23-7.18 (m, 1H), 7.13 (t, J=2.2 Hz, 1H), 7.06 (dd, J=8.3, 2.6 Hz, 1H), 4.27 (t, J=6.2 Hz, 2H), 2.87 (t, J=6.2 Hz, 2H).

### Example 99B: 5-(trifluoromethyl) chroman-4-one

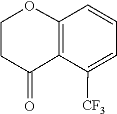 [SMILES: O=C1CCOc2cccc(C(F)(F)F)c21]

3-(3-(trifluoromethyl)phenoxy)propanoic acid (100 mg, 427.03 μmol, 1 equiv.) and polyphosphoric acid (2 mL) were added to a flame-dried tube. The mixture was stirred at 100° C. for 30 minutes. The mixture was cooled to room temperature and quenched with ice-water (5 mL). The aqueous was extracted with EA (10 mL×2). The combine organic layers were washed with brine (20 mL*3), dried over Na₂SO₄, filtrated and concentrated under reduce pressure. The crude product was purified by prep-TLC (PE:EA=20:1) to afford the title compound (10 mg, 11%) as yellow solid. ¹H NMR (400 MHz, Chloroform-d) δ 7.51 (ddq, J=8.5, 7.7, 0.8 Hz, 1H), 7.41 (ddt, J=7.6, 1.4, 0.7 Hz, 1H), 7.20 (ddd, J=8.4, 1.3, 0.6 Hz, 1H), 4.57-4.52 (m, 2H), 2.91-2.83 (m, 2H).

### Example 99C: 9-(trifluoromethyl)-4H-chromeno[4,3-d]thiazol-2-amine

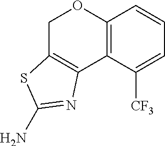 [SMILES: Nc1nc2c(s1)COc1cccc(C(F)(F)F)c1-2]

5-(trifluoromethyl) chroman-4-one (300 mg, 1.39 mmol, 1 equiv.) and hydroxy (tosyloxy) iodobenzene (653.20 mg, 1.67 mmol, 1.2 equiv.) were added to a flame-dried three necked flask. ACN (3 mL) was added to the mixture. The mixture was stirred at reflux for 45 minutes then cooled the mixture to room temperature. thiourea (126.8 mg, 1.67 mmol, 1.2 equiv.) was added to the mixture and the mixture was stirred at reflux for 4 hours. The mixture was cooled to room temperature and quenched with ice-water (20 mL). The aqueous phase was extracted with EA (20 mL×2). The combine organic layers were washed with brine (20 mL×2), dried over Na₂SO₄, filtrated, and concentrated under reduce pressure. The crude product was purified by prep-TLC (PE:EA=2:1) to afford the title compound (30 mg, 7.9%) as yellow solid. ¹H NMR (400 MHz, Chloroform-d) δ 7.39-7.34 (m, 1H), 7.21 (dt, J=7.8, 0.9 Hz, 1H), 7.13 (dd, J=8.1, 1.3 Hz, 1H), 5.19 (s, 2H), 4.91 (s, 2H).

### Example 99D: 4,6-dimethoxy-N—(9-(trifluoromethyl)-4H-chromeno[4,3-d]thiazol-2-yl)pyrimidine-5-carboxamide

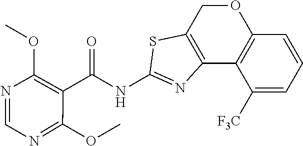 [SMILES: COc1ncnc(OC)c1C(=O)Nc1nc2c(s1)COc1cccc(C(F)(F)F)c1-2]

4,6-dimethoxypyrimidine-5-carboxylic acid (20 mg, 108.61 μmol, 1.0 equiv.) was added to a flame-dried three necked flask. DCM (0.3 mL) was added to the mixture and the reaction was cooled to 0° C. (COCl)₂ (41.3 mg, 27.57 μl, 325.82 μmol, 3.0 equiv.) was diluted with DCM (0.1 mL) and added to the mixture at 0° C. DMF (0.79 mg, 10.86 μmol, 0.1 equiv.) was diluted with DCM (0.1 mL) and added to the mixture at 0° C. The mixture was stirred at 0° C. for 2 hours. The mixture was concentrated under reduced pressure to obtain the crude product, which was used directly for the next step. 9-(trifluoromethyl)-4H-chromeno[4,3-d]thiazol-2-amine (20.1 mg, 73.84 μmol, 0.68 equiv.) was added to the residue and purged with Ar for 3 times. THF (0.4 mL) was added to the mixture and mixture was cooled to 0° C. KOH (18.3 mg, 325.77 μmol, 3equiv.) was added to the mixture at 0° C. The mixture was stirred at rt for 16 hours and quenched with ice-water (10 mL). The aqueous layer was extracted with EA (20 mL×2). The combine organic layers were washed with brine (20 mL×2), dried over Na₂SO₄, filtrated, and concentrated under reduce pressure to get the crude product. The crude product was purified by prep-TLC (PE:EA=3:1) to get the desire product (0.6 mg, 1.9%) as white solid. ESI-MS m/z=439.01 [M+H]⁺; Calculated MW: 438.06. ¹H NMR (400 MHz, Chloroform-d) δ 10.08 (s, 1H), 8.48 (s, 1H), 7.40 (dd, J=8.0, 1.3 Hz, 1H), 7.28 (d, J=6.5 Hz, 1H), 7.20-7.15 (m, 1H), 5.34 (s, 2H), 4.10 (s, 6H).

### Example 100: N—(4,4-bis(fluoromethyl)-7-(trifluoromethyl)-4H-chromeno[4,3-d]thiazol-2-yl)-4,6-dimethoxypyrimidine-5-carboxamide

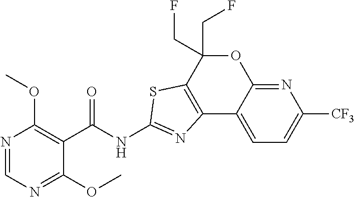 [SMILES: COc1ncnc(OC)c1C(=O)Nc1nc2c(s1)C(CF)(CF)Oc1nc(C(F)(F)F)ccc1-2]

### Example 100A: 2,2-bis(fluoromethyl)-7-(trifluoromethyl) chroman-4-one

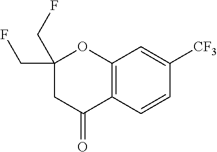 [SMILES: O=C1CC(CF)(CF)Oc2cc(C(F)(F)F)ccc21]

To a stirred solution of 1-(2-hydroxy-4-(trifluoromethyl)phenyl)ethan-1-one (200 mg, 0.978 mmol, 1.00 equiv.) and 1,3-difluoropropan-2-one (92.1 mg, 0.978 mmol, 1.00 equiv.) in MeOH (2 mL) was added pyrrolidine (139.4 mg, 1.956 mmol, 2.00 equiv.) at 0° C. The resulting mixture was stirred for 5 h at room temperature. The mixture was concentrated under reduced pressure. The crude product was purified by silica gel column chromatography (PE:EA=4:1) to afford the title compound (105 mg, 38%) as a yellow solid. ¹H NMR (400 MHz, Chloroform-d) δ 7.97 (dq, J=8.1, 0.8 Hz, 1H), 7.32-7.27 (m, 2H), 4.67-4.63 (m, 2H), 4.55-4.52 (m, 2H), 2.98 (t, J=1.3 Hz, 2H).

### Example 100B: 4,4-bis(fluoromethyl)-7-(trifluoromethyl)-4H-chromeno[4,3-d]thiazol-2-amine

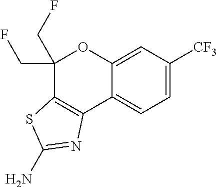 [SMILES: Nc1nc2c(s1)C(CF)(CF)Oc1cc(C(F)(F)F)ccc1-2]

To a suspension of 2,2-bis(fluoromethyl)-7-(trifluoromethyl) chroman-4-one (100 mg, 0.357 mmol, 1.00 equiv.) and TsOH-H₂O (6.8 mg, 0.0357 mmol, 0.10 equiv.) in n-hexane (2.5 mL), pyrrolidine (127 mg, 1.785 mmol, 5.00 equiv.) was added at room temperature, and the mixture was stirred for 2 h at 80° C. Then the reaction mixture was concentrated in vacuo and dissolved in MeOH (1 mL). S (28.6 mg, 0.892 mmol, 2.5 equiv.) was added at 0° C. and the mixture was stirred for 5 mins at 0° C. Then 51% w/w % cyanamide (75.0 mg, 0.892 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was purified by silica gel column chromatography (PE:EA=3:1) to afford the title compound (20 mg, 17%) as a yellow solid. ¹H NMR (400 MHz, Chloroform-d) δ 7.69 (dd, J=7.9, 1.1 Hz, 1H), 7.29-7.26 (m, 1H), 7.20-7.17 (m, 1H), 4.79-4.74 (m, 1H), 4.67-4.62 (m, 2H), 4.54 (dd, J=9.8, 2.6 Hz, 1H).

### Example 100C: N—(4,4-bis(fluoromethyl)-7-(trifluoromethyl)-4H-chromeno[4,3-d]thiazol-2-yl)-4,6-dimethoxypyrimidine-5-carboxamide

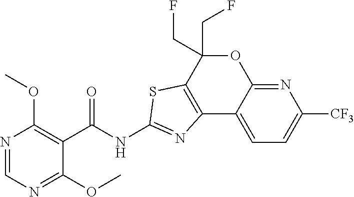 [SMILES: COc1ncnc(OC)c1C(=O)Nc1nc2c(s1)C(CF)(CF)Oc1nc(C(F)(F)F)ccc1-2]

To a suspension of 4,4-bis(fluoromethyl)-7-(trifluoromethyl)-4H-chromeno[4,3-d]thiazol-2-amine (20 mg, 0.059 mmol, 1.00 equiv.) and 4,6-dimethoxypyrimidine-5-carboxylic acid (17 mg, 0.088 mmol, 1.50 equiv.) in ACN (0.5 mL) and NMI (17 mg, 0.206 mmol, 3.50 equiv.), TCFH (25 mg, 0.088 mmol, 1.50 equiv.) was added dropwise at 0° C., and the mixture was stirred for 16 h at 50° C. Then the reaction mixture was concentrated in vacuo, the resulting mixture was extracted with EA (2×10 mL) and washed with H₂O (1×5 mL). The combined organic layers were washed with brine (1×10 mL) and dried over anhydrous Na₂SO₄. After filtration, the filtrate was concentrated under reduced pressure. The crude product was purified by prep-TLC (PE:EA=2:1) to afford the title compound (2.3 mg, 7.7%) as a white solid. ESI-MS m/z=502.95 [M+H]⁺; Calculated MW: 502.41. ¹H NMR (400 MHz, DMSO-d₆) δ 13.05 (s, 1H), 8.59 (s, 1H), 7.75 (d, J=7.9 Hz, 1H), 7.45-7.27 (m, 2H), 4.96-4.86 (m, 2H), 4.85-4.73 (m, 2H), 3.92 (s, 6H).

### Example 101:2-(4,6-dimethoxypyrimidine-5-carboxamido)-7-(trifluoromethyl)spiro[chromeno[4,3-d]thiazole-4,1′-cyclohexane]-4′-carboxylic acid

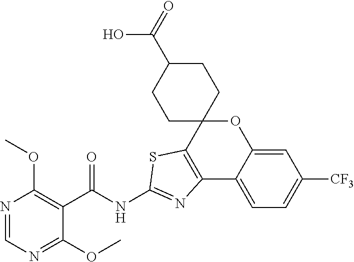 [SMILES: COc1ncnc(OC)c1C(=O)Nc1nc2c(s1)C1(CCC(C(=O)O)CC1)Oc1cc(C(F)(F)F)ccc1-2]

Ethyl 2-(4,6-dimethoxypyrimidine-5-carboxamido)-7-(trifluoromethyl)spiro[chromeno[4,3-d]thiazole-4,1′-cyclohexane]-4′-carboxylate (145 mg, 0.25 mmol, 1 equiv.) was added to a flame-dried three necked flask. THF (1.45 mL) was added to the mixture and cooled to 0° C. LiOH (17.98 mg, 0.751 mmol, 3.0 equiv.) was dissolved in H₂O (1.45 mL) and added to the mixture at 0° C. The mixture was stirred at rt for 16 hours. The mixture was quenched with ice-water (20 mL) then acidified to pH=5~6 with 1 N HCl (~0.751 mL). The aqueous was extracted with EA (20 mL×2). The combine organic layers were washed with brine (20 mL×2), dried over Na₂SO₄, filtrated, and concentrated under reduce pressure to get the desire product (130 mg, 94%) as white-off solid. ESI-MS m/z=550.89 [M+H]⁺; Calculated MW: 550.11. ¹H NMR (400 MHz, DMSO-d₆) δ 12.81 (s, 1H), 12.11 (s, 1H), 8.58 (s, 1H), 7.70 (d, J=8.0 Hz, 1H), 7.41-7.32 (m, 1H), 7.29 (d, J=1.8 Hz, 1H), 3.92 (s, 6H), 2.50-2.27 (m, 1H), 2.25-2.19 (m, 2H), 1.82-1.75 (m, 6H).

### Example 102:2-(4,6-dimethoxypyrimidine-5-carboxamido)-7-(trifluoromethyl)spiro[chromeno[4,3-d]thiazole-4,1′-cyclohexane]-4′-carboxamide

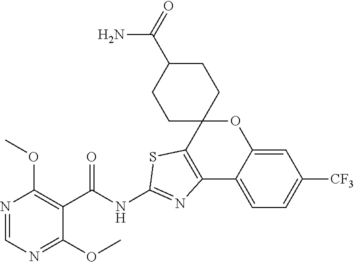 [SMILES: COc1ncnc(OC)c1C(=O)Nc1nc2c(s1)C1(CCC(C(N)=O)CC1)Oc1cc(C(F)(F)F)ccc1-2]

2-(4,6-dimethoxypyrimidine-5-carboxamido)-7-(trifluoromethyl)spiro[chromeno[4,3-d]thiazole-4,1′-cyclohexane]-4′-carboxylic acid (30 mg, 54.48 μmol, 1 equiv.) was added to a flame-dried three necked flask. DCM (0.3 mL) was added and the reaction was cooled to 0° C. (COCl)₂ (20.8 mg, 13.86 μl, 163.54 μmol, 3.0 equiv.) was diluted with DCM (0.1 mL) and added to the mixture at 0° C. DMF (0.40 mg, 5.448 μmol, 0.1 equiv.) was diluted with DCM (0.1 mL) and added to the mixture at 0° C. The mixture was stirred at 0° C. for 30 minutes followed by the addition of NH₃ (7 mol/L in MeOH, 3 mL) dropwise at 0° C. The mixture was stirred at 0° C. for 10 minutes and concentrated under reduce pressure. The residue was quenched with ice-water (10 mL). The aqueous was extracted with EA (20 mL×2). The combine organic layers were washed with brine (20 mL×2), dried over Na₂SO₄, filtered, and concentrated under reduce pressure to get the crude product. The crude product was purified by prep-TLC (EA) then purified by prep-HPLC (0.1% FA water/ACN, 30-100%, 25 minutes) to obtain the desire product (7 mg, 23%) as white solid. ESI-MS m/z=550.05 [M+H]⁺; Calculated MW: 549.12. ¹H NMR (400 MHz, Chloroform-d) δ 10.87 (s, 1H), 8.36 (s, 1H), 7.61 (d, J=7.9 Hz, 1H), 7.26 (d, J=1.6 Hz, 1H), 7.20-7.14 (m, 1H), 5.48 (d, J=35.5 Hz, 2H), 4.02 (s, 6H), 2.42 (d, J=13.8 Hz, 2H), 2.36-2.22 (m, 1H), 2.10 (qd, J=13.0, 3.4 Hz, 2H), 1.90 (d, J=13.2 Hz, 2H), 1.72 (td, J=13.7, 4.1 Hz, 2H).

### Example 103:2-(4,6-dimethoxypyrimidine-5-carboxamido)-N-methyl-7-(trifluoromethyl)spiro[chromeno[4,3-d]thiazole-4,1′-cyclohexane]-4′-carboxamide

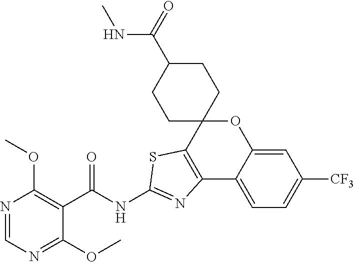 [SMILES: CNC(=O)C1CCC2(CC1)Oc1cc(C(F)(F)F)ccc1-c1nc(NC(=O)c3c(OC)ncnc3OC)sc12]

2-(4,6-dimethoxypyrimidine-5-carboxamido)-7-(trifluoromethyl)spiro[chromeno[4,3-d]thiazole-4,1′-cyclohexane]-4′-carboxylic acid (30 mg, 54.48 μmol, 1 equiv.) was added to a flame-dried three necked flask. DCM (0.3 mL) was added to the mixture and cooled the reaction to 0° C. (COCl)₂ (20.8 mg, 13.86 μl, 163.54 μmol, 3.0 equiv.) was diluted with DCM (0.1 mL) and added to the mixture at 0° C. DMF (0.40 mg, 5.448 μmol, 0.1 equiv.) was diluted with DCM (0.1 mL) and added to the mixture at 0° C. The mixture was stirred at 0° C. for 30 minutes, then the reaction mixture was added to the solution of methylamine (20% in H₂O, 3 mL) dropwise at 0° C. The mixture was stirred at 0° C. for 10 minutes and quenched with ice-water (10 mL). The aqueous phase was extracted with EA (20 mL×2) for twice. The combine organic layers were washed with brine (20 mL×2), dried over Na₂SO₄, filtrated, and concentrated under reduce pressure to get the crude product. The crude product was purified by prep-TLC (EA) then purified by prep-HPLC (0.1% FA water/ACN, 30-100%, 25 minutes) to obtain the desire product (9 mg, 29%) as white solid. ESI-MS m/z=564.06 [M+H]⁺; Calculated MW: 563.14. ¹H NMR (400 MHz, Chloroform-d) δ 10.96 (s, 1H), 8.33 (d, J=2.1 Hz, 1H), 7.59 (d, J=7.9 Hz, 1H), 7.26 (s, 1H), 7.15 (d, J=8.0 Hz, 1H), 5.53 (d, J=5.5 Hz, 1H), 3.99 (d, J=2.1 Hz, 6H), 2.86 (dd, J=5.0, 2.1 Hz, 3H), 2.41 (d, J=13.8 Hz, 2H), 2.21 (t, J=12.2 Hz, 1H), 2.09 (q, J=12.7 Hz, 2H), 1.85 (d, J=13.0 Hz, 2H), 1.77-1.66 (m, 2H).

### Example 104: ethyl 4-(2-(4,6-dimethoxypyrimidine-5-carboxamido)-4-methyl-7-(trifluoromethyl)-4H-chromeno[4,3-d]thiazol-4-yl) butanoate

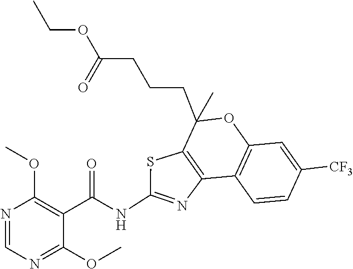 [SMILES: CCOC(=O)CCCC1(C)Oc2cc(C(F)(F)F)ccc2-c2nc(NC(=O)c3c(OC)ncnc3OC)sc21]

### Example 104A: ethyl 4-(2-methyl-4-oxo-7-(trifluoromethyl) chroman-2-yl) butanoate

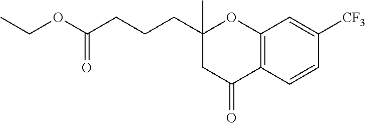 [SMILES: CCOC(=O)CCCC1(C)CC(=O)c2ccc(C(F)(F)F)cc2O1]

To a stirred solution of 1-(2-hydroxy-4-(trifluoromethyl)phenyl)ethan-1-one (1.0 g, 4.9 mmol, 1.0 equiv.) in MeOH (10.0 mL) was added ethyl 4-acetylbutyrate (1.5 g, 9.8 mmol, 2.0 equiv.) and pyrrolidine (0.7 g, 9.8 mmol, 2.0 equiv.) at 0° C. The reaction mixture was stirred overnight at 60° C. and concentrated. The resulting residue was extracted with ethyl acetate (3×100 mL). The combined organic layers were washed with brine, dried over anhydrous Na₂SO₄. The resulting mixture was concentrated under vacuum. The crude product was purified by silica gel column chromatography (PE:EA=10:1) to afford the title compound (1.1 g, 65% yield) as a yellow oil. ESI-MS m/z=345.12 [M+H]⁺; Calculated MW: 344.12;

### Example 104B: ethyl 4-(2-amino-4-methyl-7-(trifluoromethyl)-4H-chromeno[4,3-d]thiazol-4-yl) butanoate

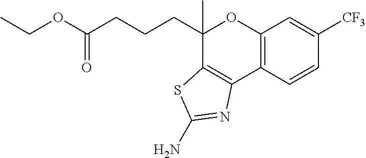 [SMILES: CCOC(=O)CCCC1(C)Oc2cc(C(F)(F)F)ccc2-c2nc(N)sc21]

To a stirred solution of ethyl 4-(2-methyl-4-oxo-7-(trifluoromethyl) chroman-2-yl) butanoate (1 g, 2.9 mmol, 1.0 equiv.) in PhMe (10 mL) was added p-TsOH (57.0 mg, 0.3 mmol, 0.1 equiv.) and pyrrolidine (1.0 g, 14.5 mmol, 5.0 equiv.) at room temperature. The reaction mixture was stirred for 2 hours at 120° C. Then it was cooled to room temperature and concentrated. Then the resulting mixture was dissolved in methanol (10.0 mL) and sulfur (233.6 mg, 7.3 mmol, 2.5 equiv.) was added. The mixture was stirred for 10 minutes at room temperature. Then cyanamide was added (601.2 mg, 7.3 mmol, 2.5 equiv.) and the mixture was stirred overnight at room temperature. After concentration, he resulting residue was extracted with ethyl acetate (3×100 mL). The combined organic layers were washed with brine, dried over anhydrous Na₂SO₄. The resulting mixture was concentrated under vacuum. The crude product was purified by prep-TLC (DCM/MeOH=30:1) to afford the title compound (230 mg, 38% yield) as a yellow oil. ESI-MS m/z=401.12 [M+H]⁺; Calculated MW: 400.11.

### Example 104C: ethyl 4-(2-(4,6-dimethoxypyrimidine-5-carboxamido)-4-methyl-7-(trifluoromethyl)-4H-chromeno[4,3-d]thiazol-4-yl) butanoate

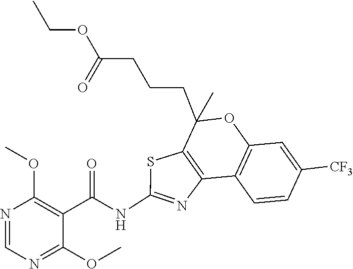 [SMILES: CCOC(=O)CCCC1(C)Oc2cc(C(F)(F)F)ccc2-c2nc(NC(=O)c3c(OC)ncnc3OC)sc21]

To a stirred solution of ethyl 4-(2-amino-4-methyl-7-(trifluoromethyl)-4H-chromeno[4,3-d]thiazol-4-yl) butanoate (15 mg, 0.027 mmol, 1.00 equiv.) in EtOH (1.00 mL) was added EtONa/EtOH (0.1 mL, 7M solution) dropwise at 0° C. The resulting mixture was stirred for 1 h at 0° C. under argon atmosphere. The reaction was quenched with sat. NH₄Cl(aq.) at 0° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (11.0 mg, 73% yield) as a light yellow solid. ESI-MS m/z=567.02 [M+H]⁺; Calculated MW: 566.14; ¹H NMR (400 MHz, Chloroform-d) δ 10.92 (s, 1H), 8.34 (s, 1H), 7.58 (dd, J=8.4, 0.9 Hz, 1H), 7.12 (q, J=2.8, 1.6 Hz, 2H), 4.10 (q, J=7.1 Hz, 2H), 4.00 (s, 6H), 2.32 (t, J=7.3 Hz, 2H), 2.05-1.72 (m, 4H), 1.68 (s, 3H), 1.22 (t, J=7.1 Hz, 3H).

### Example 105: N—(7-(difluoromethyl)-4,4-diethyl-4H-chromeno[4,3-d]thiazol-2-yl)-4,6-dimethoxypyrimidine-5-carboxamide

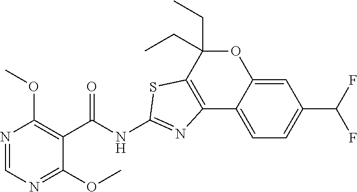 [SMILES: CCC1(CC)Oc2cc(C(F)F)ccc2-c2nc(NC(=O)c3c(OC)ncnc3OC)sc21]

N—(4,4-diethyl-7-formyl-4H-chromeno[4,3-d]thiazol-2-yl)-4,6-dimethoxypyrimidine-5-carboxamide (30 mg, 66.01 μmol, 1 equiv.) was added to a flame-dried three necked flask. The mixture was evacuated and back-filled with Ar for 3 times. DCM (1 mL) was added and the mixture was cooled to 0° C. DAST (266 mg, 0.218 mL, 1.65 mmol, 25 equiv.) was diluted with DCM (1 mL) and added to the mixture dropwise at 0° C. The mixture was stirred at 0° C. to rt for 16 hours. The mixture was quenched with sat. NaHCO₃ aqueous solution (10 mL). The aqueous phase was extracted with EA (20 mL×2). The combine organic layers were washed with brine (20 mL×2), dried over Na₂SO₄, filtrated, and concentrated under reduce pressure to get the crude product. The crude product was purified by prep-TLC (PE:EA=2:1) to afford the desire product (10 mg, 32%) as yellow solid. ESI-MS m/z=477.01. [M+H]⁺; Calculated MW: 476.13. ¹H NMR (400 MHz, Chloroform-d) δ 11.20 (s, 1H), 8.30 (s, 1H), 7.52 (d, J=7.8 Hz, 1H), 7.00 (s, 1H), 6.97 (d, J=7.8 Hz, 1H), 6.56 (t, J=56.5 Hz, 1H), 3.99 (s, 6H), 1.95 (ddq, J=47.3, 14.6, 7.4 Hz, 4H), 0.99 (t, J=7.4 Hz, 6H).

### Example 106: tert-butyl 2-(4,6-dimethoxypyrimidine-5-carboxamido)-7-(trifluoromethyl)spiro[chromeno[4,3-d]thiazole-4,3′-pyrrolidine]-1′-carboxylate

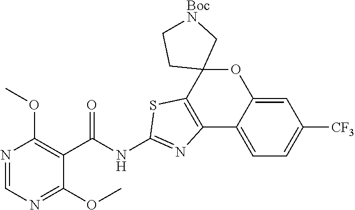 [SMILES: COc1ncnc(OC)c1C(=O)Nc1nc2c(s1)C1(CCN(C(=O)OC(C)(C)C)C1)Oc1cc(C(F)(F)F)ccc1-2]

### Example 106A: tert-butyl 4-oxo-7-(trifluoromethyl)spiro[chromane-2,3′-pyrrolidine]-1′-carboxylate

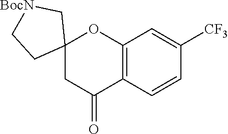 [SMILES: CC(C)(C)OC(=O)N1CCC2(CC(=O)c3ccc(C(F)(F)F)cc3O2)C1]

To a stirred solution of 1-(2-hydroxy-4-(trifluoromethyl)phenyl)ethan-1-one (1 g, 4.90 mmol, 1.00 equiv.) and tert-butyl 3-oxopyrrolidine-1-carboxylate (1.81 g, 9.80 mmol, 2.0 equiv.) in MeOH (10 mL) was added pyrrolidine (696.78 mg, 0.817 mL, 9.80 mmol, 2.00 equiv.) at 0° C. The mixture was stirred at rt for 16 hours. The mixture was quenched with ice-water and extracted with EA (20 mL×2). The combined organic layers were washed by brine (20 mL×2), dried over Na₂SO₄, filtrated, and concentrated under reduce pressure. The residue was purified by prep-TLC (PE:EA=3:1) to produce the title compound (1.56 g, 86%) as yellow oil. ¹H NMR (400 MHz, Chloroform-d) δ 7.97 (dd, J=8.6, 3.5 Hz, 1H), 7.26-7.20 (m, 2H), 3.71-3.31 (m, 4H), 3.01-2.84 (m, 2H), 2.29 (ddt, J=13.4, 6.5, 2.1 Hz, 1H), 1.93 (tt, J=13.0, 9.6 Hz, 1H), 1.43 (s, 9H).

### Example 106B: tert-butyl 2-amino-7-(trifluoromethyl)spiro[chromeno[4,3-d]thiazole-4,3′-pyrrolidine]-1′-carboxylate

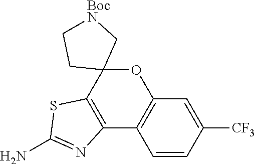 [SMILES: CC(C)(C)OC(=O)N1CCC2(C1)Oc1cc(C(F)(F)F)ccc1-c1nc(N)sc12]

To a suspension of tert-butyl 4-oxo-7-(trifluoromethyl)spiro[chromane-2,3′-pyrrolidine]-1′-carboxylate (1 g, 2.69 mmol, 1.00 equiv.) and TsOH-H₂O (102.4 mg, 538.57 μmol, 0.2 equiv.) in toluene (20 mL), pyrrolidine (957.6 mg, 1.12 mL, 13.46 mmol, 5.00 equiv.) was added at room temperature, and the mixture was stirred at reflux for 2 hours. Then the reaction mixture was cooled to rt and concentrated in vacuo and dissolved in MeOH (20 mL). S (215.83 mg, 6.73 mmol, 2.5 equiv.) was added at 0° C. and the mixture was stirred for 15 mins at 0° C. Then 51% w/w % cyanamide (566.1 mg, 0.442 mL, 6.73 mmol, 2.5 equiv.) was added at 0° C. and the mixture was stirred overnight at room temperature. The resulting mixture was concentrated under vacuum and was extracted with EA (2×30 mL). The combined organic layers were washed with brine (1×30 mL), dried over anhydrous Na₂SO₄. After filtration, the filtrate was concentrated under reduced pressure. The crude product was purified by silica gel column chromatography (PE:EA=3:1) to afford the title compound (303 mg, 26%) as a yellow solid. ¹H NMR (400 MHz, Chloroform-d) δ 7.65 (d, J=7.9 Hz, 1H), 7.23 (d, J=7.9 Hz, 1H), 7.14 (s, 1H), 5.20 (d, J=7.8 Hz, 2H), 3.67-3.39 (m, 4H), 2.55-2.48 (m, 1H), 2.13-2.05 (m, 1H), 1.46 (s, 9H).

### Example 106C: tert-butyl 2-(4,6-dimethoxypyrimidine-5-carboxamido)-7-(trifluoromethyl)spiro[chromeno[4,3-d]thiazole-4,3′-pyrrolidine]-1′-carboxylate

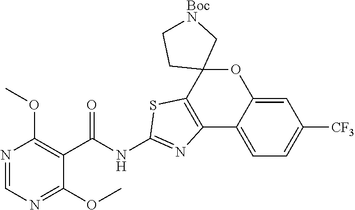 [SMILES: COc1ncnc(OC)c1C(=O)Nc1nc2c(s1)C1(CCN(C(=O)OC(C)(C)C)C1)Oc1cc(C(F)(F)F)ccc1-2]

Tert-butyl 2-amino-7-(trifluoromethyl)spiro[chromeno[4,3-d]thiazole-4,3′-pyrrolidine]-1′-carboxylate (50 mg, 116.97 μmol, 1 equiv.) and 4,6-dimethoxypyrimidine-5-carboxylic acid (32.31 mg, 175.46 μmol, 1.5 equiv.) were added to a flame-dried three necked flask. The flask was evacuated and back-filled with Ar for 3 times. ACN (1 mL) was added and the mixture was cooled to 0° C. NMI (33.62 mg, 32.32 ul, 409.41 μmol, 3.5 equiv.) was added to the mixture at 0° C. TCFH (49.2 mg, 175.46 μmol, 1.5 equiv.) was dissolved in 0.5 mL ACN and added to the mixture at 0° C. The mixture was stirred at rt for 3 hours then stirred at 50° C. for 16 hours. The mixture was concentrated under reduce pressure then quenched with ice-water (10 mL). The aqueous phase was extracted with EA (10 mL×2). The combine organic layers were washed with brine (20 mL×2), dried over Na₂SO₄, filtrated, and concentrated under reduce pressure. The crude product was purified by prep-TLC eluted with PE:EA=3:1 to afford the title compound (17 mg, 24%) as white solid. ESI-MS m/z=594.03 [M+H]⁺; Calculated MW: 593.15. ¹H NMR (400 MHz, Chloroform-d) δ 10.44 (d, J=22.0 Hz, 1H), 8.45 (d, J=5.5 Hz, 1H), 7.72 (t, J=7.5 Hz, 1H), 7.19 (s, 1H), 4.09 (d, J=4.4 Hz, 6H), 4.05-3.90 (m, 1H), 3.78-3.65 (m, 2H), 3.56 (d, J=13.0 Hz, 1H), 2.57 (d, J=13.7 Hz, 1H), 2.19 (td, J=22.6, 10.1 Hz, 1H), 1.48 (d, J=17.1 Hz, 9H).

### Example 107: N—(4,4-diethyl-4H-thiazolo[5′,4′: 4,5]pyrano[2,3-c]pyridin-2-yl)-4,6-dimethoxypyrimidine-5-carboxamide

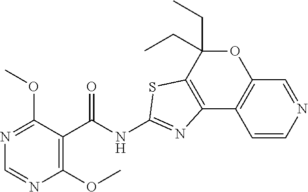 [SMILES: CCC1(CC)Oc2cnccc2-c2nc(NC(=O)c3c(OC)ncnc3OC)sc21]

### Example 107A: 2,2-diethyl-2,3-dihydro-4H-pyrano[2,3-c]pyridin-4-one

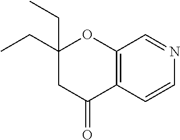

To a suspension of 1-(3-hydroxypyridin-4-yl)ethan-1-one (70 mg, 0.5104 mmol, 1.00 equiv.) in MeOH (2 mL), 3-Pentanone (440 mg, 5.1043 mmol, 10.00 equiv.), 4A molecular sieves (0.625 g) and pyrrolidine (72.5 mg, 1.0208 mmol, 2.00 equiv.) was added at 0° C. and the mixture was stirred overnight at room temperature. The resulting mixture was concentrated under vacuum and was extracted with EA (2×5 mL). The combined organic layers were washed with brine (2×5 mL), dried over anhydrous Na₂SO₄. After filtration, the filtrate was concentrated under reduced pressure. The crude product was purified by silica gel column chromatography (PE:EA=4:1) to afford the title compound (35 mg, 33%) as a yellow oil. ¹H NMR (400 MHz, Chloroform-d) δ 8.47 (d, J=0.7 Hz, 1H), 8.26 (d, J=5.0 Hz, 1H), 7.57 (dd, J=5.0, 0.7 Hz, 1H), 2.77 (s, 2H), 1.86-1.66 (m, 4H), 0.93 (t, J=7.5 Hz, 6H).

### Example 107B: 4,4-diethyl-4H-thiazolo[4′,5′: 4,5]pyrano[2,3-c]pyridin-2-amine

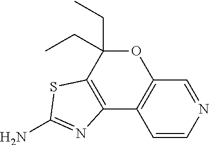 [SMILES: CCC1(CC)Oc2cnccc2-c2nc(N)sc21]

To a suspension of 2,2-diethyl-2,3-dihydro-4H-pyrano[2,3-c]pyridin-4-one (35 mg, 0.1705 mmol, 1.00 equiv.) and TsOH-H₂O (3.2 mg, 0.0171 mmol, 0.10 equiv.) in n-hexane (2 mL), pyrrolidine (60.5 mg, 0.8526 mmol, 5.00 equiv.) was added at 0° C., and the mixture was stirred for 2 h at 80° C. Then the reaction mixture was concentrated in vacuo and dissolved in MeOH (2 mL). S (13.7 mg, 0.4263 mmol, 2.5 equiv.) was added at 0° C. and the mixture was stirred for 5 mins at 0° C. Then 51% w/w % cyanamide (35.1 mg, 0.4263 mmol, 2.5 equiv.) was added at 0° C. and the mixture was stirred overnight at room temperature. The resulting mixture was concentrated under vacuum and was extracted with EA (2×5 mL). The combined organic layers were washed with brine (2×5 mL), dried over anhydrous Na₂SO₄. After filtration, the filtrate was concentrated under reduced pressure. The crude product was purified by silica gel column chromatography (PE:EA=10:1) to afford the title compound (6 mg, 14%) as a yellow solid. ESI-MS m/z=261.93 [M+H]⁺; Calculated MW: 261.34.

### Example 107C: N—(4,4-diethyl-4H-thiazolo[5′,4′: 4,5]pyrano[2,3-c]pyridin-2-yl)-4,6-dimethoxypyrimidine-5-carboxamide

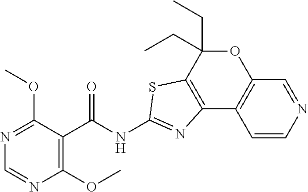 [SMILES: CCC1(CC)Oc2cnccc2-c2nc(NC(=O)c3c(OC)ncnc3OC)sc21]

To a stirred solution of 4,4-diethyl-4H-thiazolo[4′,5′: 4,5]pyrano[2,3-c]pyridin-2-amine (7.8 mg, 0.0299 mmol, 1.00 equiv.), 4,6-dimethoxypyrimidine-5-carboxylic acid (8.3 mg, 0.0449 mmol, 1.50 equiv.) and NMI (8.6 mg, 0.1047 mmol, 3.50 equiv.) in ACN (1 mL) was added TCFH (16.8 mg, 0.0598 mmol, 2.00 equiv.) in ACN (1 mL) at 0° C., and the mixture was stirred for 2 days at room temperature. The mixture was quenched with NaHCO₃(aq), extracted with EA (2×5 mL) and washed with brine. The combined organic layers were washed with brine (3×5 mL), dried over anhydrous Na₂SO₄. After filtration, the filtrate was concentrated under reduced pressure. The crude product was purified by silica gel chromatography (DCM:PE:EA=1:1:1) to afford the title compound (1.3 mg, 10%) as a white solid. ESI-MS m/z=428.06 [M+H]⁺; Calculated MW: 427.48. ¹H NMR (400 MHz, Chloroform-d) δ 10.27 (s, 1H), 8.50 (s, 1H), 8.21 (d, J=19.9 Hz, 2H), 7.53 (s, 1H), 4.13 (s, 6H), 2.10-1.96 (m, 4H), 0.97 (t, J=7.4 Hz, 6H).

### Example 108: N—(2′,2′-dimethyl-7-(trifluoromethyl)spiro[chromeno[4,3-d]thiazole-4,5′-[1,3]dioxan]-2-yl)-4,6-dimethoxypyrimidine-5-carboxamide

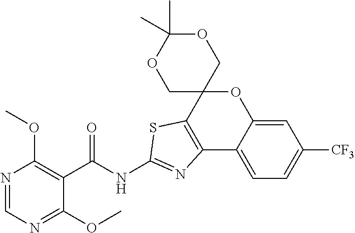 [SMILES: COc1ncnc(OC)c1C(=O)Nc1nc2c(s1)C1(COC(C)(C)OC1)Oc1cc(C(F)(F)F)ccc1-2]

### Example 108A: 1-(2′,2′-dimethyl-7-(trifluoromethyl)spiro[chromene-2,5′-[1,3]dioxan]-4-yl)pyrrolidine

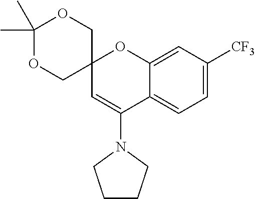 [SMILES: CC1(C)OCC2(C=C(N3CCCC3)c3ccc(C(F)(F)F)cc3O2)CO1]

To a stirred solution of 1-(2-hydroxy-4-(trifluoromethyl)phenyl)ethan-1-one (720 mg, 3.529 mmol, 1.00 equiv.) in MeOH (5 mL) was added pyrrolidine (139 mg, 7.059 mmol, 2.00 equiv.) at 0° C., and the mixture was stirred for 2 h at room temperature. Then 2,2-dimethyl-1,3-dioxan-5-one (505 mg, 3.882 mmol, 1.1 equiv.) was added at 0° C., and the mixture was stirred overnight at room temperature. The precipitated product was filtered, washed with PE and dried under vacuum to afford the title compound as a yellow solid (500 mg, 38%). ¹H NMR (400 MHz, Chloroform-d) δ 7.42 (d, J=8.1 Hz, 1H), 7.27 (d, J=1.9 Hz, 1H), 7.19-7.12 (m, 1H), 4.57 (s, 1H), 3.94-3.86 (m, 2H), 3.82-3.74 (m, 2H), 3.09-3.01 (m, 4H), 1.96-1.85 (m, 4H), 1.47 (d, J=13.6 Hz, 6H).

### Example 108B: 2′,2′-dimethyl-7-(trifluoromethyl)spiro[chromeno[4,3-d]thiazole-4,5′-[1,3]dioxan]-2-amine

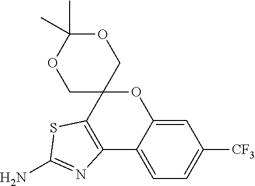 [SMILES: CC1(C)OCC2(CO1)Oc1cc(C(F)(F)F)ccc1-c1nc(N)sc12]

To a suspension of 1-(2′,2′-dimethyl-7-(trifluoromethyl)spiro[chromene-2,5′-[1,3]dioxan]-4-yl)pyrrolidine (250 mg, 0.678 mmol, 1.00 equiv.) in MeOH (3 mL) and THF (2 mL), S (54 mg, 1.694 mmol, 2.5 equiv.) was added at 0° C. and the mixture was stirred for 1 h at room temperature. Then 51% w/w % cyanamide (139 mg, 1.694 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the filtrate was concentrated under reduced pressure. The crude product was re-crystallized from DCM/EtOAc/PE in the ratio of 1:1:6 to afford the title compound (250 mg, 99%) as a yellow solid. ¹H NMR (400 MHz, Chloroform-d) δ 7.67 (dd, J=8.0, 1.0 Hz, 1H), 7.22 (ddd, J=7.9, 1.8, 0.8 Hz, 1H), 7.14 (d, J=1.7 Hz, 1H), 5.04 (s, 2H), 4.12-4.01 (m, 2H), 3.99-3.88 (m, 2H), 1.56 (d, J=11.9 Hz, 6H).

### Example 108C: N—(2′,2′-dimethyl-7-(trifluoromethyl)spiro[chromeno[4,3-d]thiazole-4,5′-[1,3]dioxan]-2-yl)-4,6-dimethoxypyrimidine-5-carboxamide

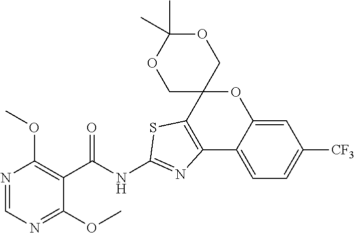 [SMILES: COc1ncnc(OC)c1C(=O)Nc1nc2c(s1)C1(COC(C)(C)OC1)Oc1cc(C(F)(F)F)ccc1-2]

To a suspension of 4,6-dimethoxypyrimidine-5-carboxylic acid (8 mg, 0.0403 mmol, 1.50 equiv.) in CH₂Cl₂ (0.3 mL) and catalytic amount of DMF, oxalyl chloride (10 mg, 0.0806 mmol, 2.0 equiv.) was added dropwise at 0° C., and the mixture was stirred for 3 h at room temperature. Then the reaction mixture was concentrated in vacuo and dissolved in THF (0.5 mL). 2′,2′-dimethyl-7-(trifluoromethyl)spiro[chromeno[4,3-d]thiazole-4,5′-[1,3]dioxan]-2-amine (10 mg, 0.0269 mmol, 1.0 equiv.), KOH (5 mg, 0.0806 mmol, 4.0 equiv.) were added, and the mixture was stirred overnight at room temperature. Then the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by prep-TLC (DCM:PE:EA=1:3:1) to afford the title compound (6 mg, 41%) as a white solid. ESI-MS m/z=538.97 [M+H]⁺; Calculated MW: 538.11. ¹H NMR (400 MHz, Chloroform-d) δ 11.08 (s, 1H), 8.31 (s, 1H), 7.61 (d, J=7.9 Hz, 1H), 7.22-7.12 (m, 2H), 4.13 (d, J=11.5 Hz, 2H), 4.06-4.01 (m, 2H), 3.98 (s, 6H), 1.58 (d, J=1.2 Hz, 6H).

### Example 109: tert-butyl(2-(4,6-dimethoxypyrimidine-5-carboxamido)-7-(trifluoromethyl)spiro[chromeno[3,4-d]thiazole-4,1′-cyclohexan]-4′-yl)carbamate

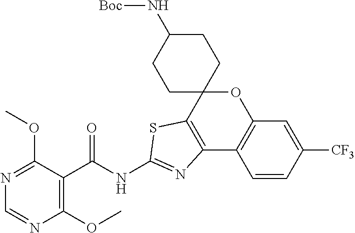 [SMILES: COc1ncnc(OC)c1C(=O)Nc1nc2c(s1)C1(CCC(NC(=O)OC(C)(C)C)CC1)Oc1cc(C(F)(F)F)ccc1-2]

### Example 109A: tert-butyl (4-oxo-7-(trifluoromethyl)spiro[chromane-2,1′-cyclohexan]-4′-yl)carbamate

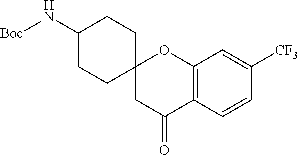 [SMILES: CC(C)(C)OC(=O)NC1CCC2(CC1)CC(=O)c1ccc(C(F)(F)F)cc1O2]

To a suspension of 1-(2-hydroxy-4-(trifluoromethyl)phenyl)ethan-1-one (200 mg, 0.9804 mmol, 1.00 equiv.) in MeOH (2 mL), tert-butyl (4-oxocyclohexyl)carbamate (418 mg, 1.9608 mmol, 2.00 equiv.) and pyrrolidine (139.2 mg, 1.9608 mmol, 2.00 equiv.) was added at 0° C. and the mixture was stirred overnight at room temperature. The resulting mixture was concentrated under vacuum and was extracted with EA (2×3 mL). The combined organic layers were washed with brine (3×5 mL), dried over anhydrous Na₂SO₄. After filtration, the filtrate was concentrated under reduced pressure and passed through silica gel pad (PE:EA=4:1) to afford the title compound (423 mg, crude) as a white solid. ¹H NMR (400 MHz, Chloroform-d) δ 7.98-7.90 (m, 1H), 7.23 (d, J=2.4 Hz, 1H), 7.22-7.18 (m, 1H), 3.60 (d, J=86.8 Hz, 1H), 2.76 (d, J=41.5 Hz, 2H), 2.13 (d, J=12.1 Hz, 1H), 2.03-1.76 (m, 4H), 1.56-1.45 (m, 3H), 1.43 (d, J=2.3 Hz, 9H).

### Example 109B: tert-butyl(2-amino-7-(trifluoromethyl)spiro[chromeno[4,3-d]thiazole-4,1′-cyclohexan]-4′-yl)carbamate

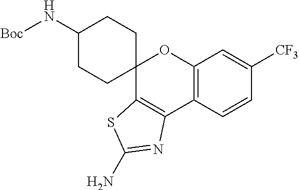 [SMILES: CC(C)(C)OC(=O)NC1CCC2(CC1)Oc1cc(C(F)(F)F)ccc1-c1nc(N)sc12]

To a suspension of tert-butyl (4-oxo-7-(trifluoromethyl)spiro[chromane-2,1′-cyclohexan]-4′-yl) carbamat (159 mg, 0.3981 mmol, 1.00 equiv.) and TsOH-H₂O (0.076 mg, 0.0398 mmol, 0.10 equiv.) in n-hexane (6 mL), pyrrolidine (141.3 mg, 1.9905 mmol, 5.00 equiv.) was added at room temperature, and the mixture was stirred for 2 h at 80° C. Then the reaction mixture was concentrated in vacuo and dissolved in MeOH (5 mL). S (32 mg, 0.9953 mmol, 2.5 equiv.) was added at 0° C. and the mixture was stirred for 5 min at 0° C. Then 51% w/w % cyanamide (82 mg, 0.9953 mmol, 2.5 equiv.) was added at 0° C. and the mixture was stirred overnight at room temperature. The resulting mixture was concentrated under vacuum and was extracted with EA (2×15 mL). The combined organic layers were washed with brine (1×10 mL), dried over anhydrous Na₂SO₄. After filtration, the resulting mixture was concentrated under vacuum. The crude product was purified by silica gel column chromatography (PE:EA=5:1) to afford the title compound (30 mg, 17%) as a yellow solid. ¹H NMR (400 MHz, Chloroform-d) δ 7.66 (d, J=8.0 Hz, 1H), 7.21 (d, J=2.3 Hz, 1H), 7.15 (d, J=7.6 Hz, 1H), 5.51 (s, 2H), 4.51 (s, 1H), 3.52 (s, 1H), 2.31 (d, J=11.5 Hz, 1H), 2.16-2.04 (m, 2H), 1.88 (d, J=7.5 Hz, 2H), 1.68 (s, 3H), 1.45 (d, J=6.6 Hz, 9H).

### Example 109C: tert-butyl(2-(4,6-dimethoxypyrimidine-5-carboxamido)-7-(trifluoromethyl)spiro[chromeno[3,4-d]thiazole-4,1′-cyclohexan]-4′-yl)carbamate

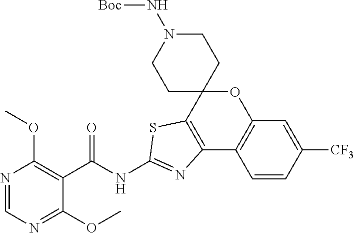 [SMILES: COc1ncnc(OC)c1C(=O)Nc1nc2c(s1)C1(CCN(NC(=O)OC(C)(C)C)CC1)Oc1cc(C(F)(F)F)ccc1-2]

To a stirred solution of tert-butyl(2-amino-7-(trifluoromethyl)spiro[chromeno[4,3-d]thiazole-4,1′-cyclohexan]-4′-yl)carbamate (20 mg, 0.0440 mmol, 1.00 equiv.), 4,6-dimethoxypyrimidine-5-carboxylic acid (12 mg, 0.061 mmol, 1.50 equiv.) and NMI (12.6 mg, 0.1540 mmol, 3.50 equiv.) in ACN (1 mL) was added TCFH (24.7 mg, 0.0880 mmol, 2.00 equiv.) in ACN (1 mL) at 0° C., and the mixture was stirred for overnight at 50° C. The mixture was quenched with NaHCO₃(aq), then the resulting mixture was extracted with EA (2×5 mL). The combined organic layers were washed with brine (3×5 mL), dried over anhydrous Na₂SO₄. After filtration, the filtrate was concentrated under reduced pressure. The crude product was purified by silica gel chromatography (DCM:PE:EA=1:2:1) to afford the title compound (5 mg, 18%) as a white solid. ESI-MS m/z=622.05 [M+H]⁺; Calculated MW: 621.19. ¹H NMR (400 MHz, Chloroform-d) δ 11.02 (s, 1H), 8.32 (dd, J=4.1, 1.5 Hz, 1H), 7.59 (dd, J=7.7, 5.0 Hz, 1H), 7.16 (t, J=8.6 Hz, 2H), 4.57 (d, J=34.5 Hz, 1H), 4.14-3.90 (m, 6H), 3.87-3.45 (m, 1H), 2.39-2.17 (m, 2H), 2.17-1.88 (m, 4H), 1.86-1.65 (m, 4H), 1.46 (d, J=7.0 Hz, 9H).

### Example 110: N—((4,6-dimethoxypyrimidin-5-yl)methyl)-4,4-diethyl-7-(trifluoromethyl)-4H-chromeno[4,3-d]thiazol-2-amine

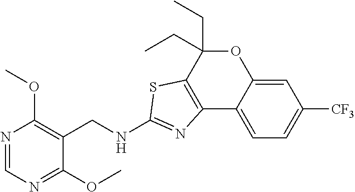 [SMILES: CCC1(CC)Oc2cc(C(F)(F)F)ccc2-c2nc(NCc3c(OC)ncnc3OC)sc21]

To a stirred solution of N—(4,4-diethyl-7-(trifluoromethyl)-4H-chromeno[4,3-d]thiazol-2-yl)-4,6-dimethoxypyrimidine-5-carboxamide (20 mg, 0.040 mmol, 1.00 equiv.) in THF (1.00 mL) was added LiAlH₄ (0.162 mL, 0.162 mmol, 4.00 equiv., 1 M in THF) at 0° C. The resulting mixture was stirred for 5 min at 0° C. under argon atmosphere. The reaction was quenched with EA at 0° C., diluted with water, extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (7 mg, 36.4% yield) as a white solid. ESI-MS m/z=497.03 [M+H]⁺; Calculated MW: 480.14; ¹H NMR (400 MHz, Chloroform-d) δ 9.82 (s, 1H), 7.68 (d, J=7.9 Hz, 1H), 7.20-7.14 (m, 1H), 7.12 (d, J=1.7 Hz, 1H), 5.30 (d, J=4.6 Hz, 2H), 3.76 (s, 6H), 2.00 (dq, J=14.7, 7.4 Hz, 2H), 1.85 (dq, J=14.7, 7.4 Hz, 2H), 0.94 (t, J=7.4 Hz, 6H).

### Example 111: 4,6-dichloro-N—(4,4-diethyl-7-(trifluoromethyl)-4H-chromeno[4,3-d]thiazol-2-yl)pyrimidine-5-carboxamide

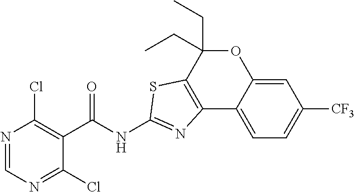 [SMILES: CCC1(CC)Oc2cc(C(F)(F)F)ccc2-c2nc(NC(=O)c3c(Cl)ncnc3Cl)sc21]

To a stirred solution of 4,6-dichloropyrimidine-5-carboxylic acid (70 mg, 0.366 mmol, 1.20 equiv.) and 4,4-diethyl-7-(trifluoromethyl)-4H-chromeno[4,3-d]thiazol-2-amine (100.0 mg, 0.305 mmol, 1.00 equiv.) in ACN (2.00 mL) was added NMI (88.0 mg, 1.07 mmol, 3.50 equiv.) and TCFH (128.0 mg, 0.457 mmol, 1.50 equiv.) in portions at 0° C. The resulting mixture was stirred for 16 h at room temperature under argon. The reaction was diluted with water, extracted with EtOAc (3×40 mL). The combined organic layers were washed with brine (2×20 mL), dried over anhydrous Na₂SO₄. After filtration, the filtrate was concentrated under reduced pressure. The residue was purified by prep-TLC (PE/EtOAc (2:1)) to afford the title compound (30 mg, 19% yield) as a light yellow solid. ESI-MS m/z=502.85 [M+H]⁺; Calculated MW: 502.02; ¹H NMR (400 MHz, Chloroform-d) δ 12.29 (s, 1H), 8.38 (s, 1H), 7.35-7.30 (m, 1H), 7.16-7.10 (m, 2H), 2.10-1.88 (m, 4H), 1.06 (t, J=7.4 Hz, 6H).

### Example 112:4-chloro-N—(4,4-diethyl-7-(trifluoromethyl)-4H-chromeno[4,3-d]thiazol-2-yl)-6-methoxypyrimidine-5-carboxamide

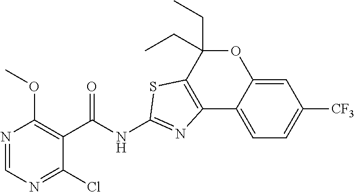 [SMILES: CCC1(CC)Oc2cc(C(F)(F)F)ccc2-c2nc(NC(=O)c3c(Cl)ncnc3OC)sc21]

To a stirred solution of 4,6-dichloro-N—(4,4-diethyl-7-(trifluoromethyl)-4H-chromeno[4,3-d]thiazol-2-yl)pyrimidine-5-carboxamide (70 mg, 0.139 mmol, 1.00 equiv.) in THF (1.00 mL) was added MeONa/MeOH (0.028 mL, 0.139 mmol, 1.00 equiv., 30% w.t.) dropwise at −10° C. The resulting mixture was stirred for 1 h at −10° C. under argon atmosphere. The reaction was quenched with sat. NH₄Cl(aq.) at −10° C., extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (60 mg, 87% yield) as a white solid. ESI-MS m/z=498.88 [M+H]⁺; Calculated MW: 498.07; ¹H NMR (400 MHz, Chloroform-d) δ 11.60 (s, 1H), 8.32 (s, 1H), 7.42 (d, J=7.8 Hz, 1H), 7.12 (d, J=1.7 Hz, 1H), 7.09-7.04 (m, 1H), 3.93 (s, 3H), 2.04 (dq, J=14.7, 7.4 Hz, 2H), 1.92 (dq, J=14.7, 7.4 Hz, 2H), 1.03 (t, J=7.4 Hz, 6H).

### Example 113: N—(4,4-bis(hydroxymethyl)-7-(trifluoromethyl)-4H-chromeno[4,3-d]thiazol-2-yl)-4,6-dimethoxypyrimidine-5-carboxamide

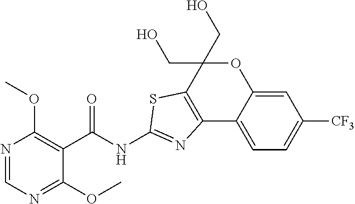 [SMILES: COc1ncnc(OC)c1C(=O)Nc1nc2c(s1)C(CO)(CO)Oc1cc(C(F)(F)F)ccc1-2]

To a stirred solution of N—(2′,2′-dimethyl-7-(trifluoromethyl)spiro[chromeno[4,3-d]thiazole-4,5′-[1,3]dioxan]-2-yl)-4,6-dimethoxypyrimidine-5-carboxamide (20 mg, 0.0372 mmol, 1.00 equiv.) in MeOH (0.2 mL) and THF (0.2 mL) was added 3N HCl (25 μL, 0.0743 mmol, 2.00 equiv.) at 0° C., and the mixture was stirred overnight at room temperature. The resulting mixture was concentrated under vacuum. The crude product was re-crystallized from DCM/EtOAc in the ratio of 1:1 to afford the title compound (8 mg, 43%) as a white solid. ESI-MS m/z=498.97 [M+H]⁺; Calculated MW: 498.08. ¹H NMR (400 MHz, DMSO-d₆) δ 12.77 (s, 1H), 8.58 (s, 1H), 7.66 (d, J=7.8 Hz, 1H), 7.30-7.23 (m, 1H), 7.14 (d, J=1.7 Hz, 1H), 3.92 (s, 6H), 3.76 (d, J=11.3 Hz, 2H), 3.62 (d, J=11.3 Hz, 2H).

### Example 114:4-(2-(4,6-dimethoxypyrimidine-5-carboxamido)-4-methyl-7-(trifluoromethyl)-4H-chromeno[4,3-d]thiazol-4-yl) butanoic acid

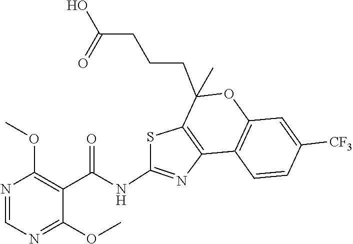 [SMILES: COc1ncnc(OC)c1C(=O)Nc1nc2c(s1)C(C)(CCCC(=O)O)Oc1cc(C(F)(F)F)ccc1-2]

To a stirred solution of ethyl 4-(2-(4,6-dimethoxypyrimidine-5-carboxamido)-4-methyl-7-(trifluoromethyl)-4H-chromeno[4,3-d]thiazol-4-yl) butanoate (20 mg, 0.035 mmol, 1.00 equiv.) in MeOH (1.00 mL) was added LiOH (15.0 mg, 0.353 mmol, 10.00 equiv.) in H₂O (0.5 mL) dropwise at 0° C. The resulting mixture was stirred for 3 h at room temperature under argon. The reaction was quenched with 2M HCl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (1:10)) to afford the title compound (11 mg, 58% yield) as a white solid. ESI-MS m/z=538.99 [M+H]⁺; Calculated MW: 538.11; ¹H NMR (400 MHz, Chloroform-d) δ 11.39 (s, 1H), 8.36 (s, 1H), 7.55 (d, J=8.1 Hz, 1H), 7.13 (d, J=7.1 Hz, 2H), 3.98 (s, 6H), 2.37 (t, J=7.3 Hz, 2H), 2.09-1.72 (m, 4H), 1.67 (s, 3H).

### Example 115: isopropyl 2′-(4,6-dimethoxypyrimidine-5-carboxamido)-7′-(trifluoromethyl)spiro[azetidine-3,4′-chromeno[4,3-d]thiazole]-1-carboxylate

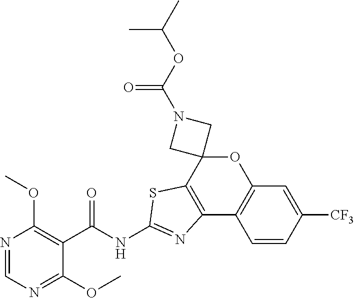 [SMILES: COc1ncnc(OC)c1C(=O)Nc1nc2c(s1)C1(CN(C(=O)OC(C)C)C1)Oc1cc(C(F)(F)F)ccc1-2]

To a stirred solution of 4,6-dimethoxy-N—(7′-(trifluoromethyl)spiro[azetidine-3,4′-chromeno[4,3-d]thiazol]-2′-yl)pyrimidine-5-carboxamide (15 mg, 0.030 mmol, 1.00 equiv.) in DCM (1.00 mL) was added TEA (33.00 mg, 0.300 mmol, 10.00 equiv.) and isopropyl carbonochloridate (6.00 mg, 0.046 mmol, 1.50 equiv.) in DCM (0.5 mL) dropwise at 0° C. The resulting mixture was stirred for 1 h at 0° C. under argon. The reaction was diluted with DCM at 0° C., washed with water (2×5 mL), dried over anhydrous Na₂SO₄. After filtration, the filtrate was concentrated under reduced pressure. The residue was purified by prep-TLC (PE/EtOAc (2:1)) to afford the title compound (9 mg, 53% yield) as a white solid. ESI-MS m/z=566.01 [M+H]⁺; Calculated MW: 565.12; ¹H NMR (400 MHz, Chloroform-d) δ 10.77 (s, 1H), 8.38 (s, 1H), 7.65 (d, J=7.8 Hz, 1H), 7.22 (d, J=8.4 Hz, 2H), 4.94 (p, J=6.2 Hz, 1H), 4.48 (dd, J=9.8, 1.1 Hz, 2H), 4.29 (dd, J=9.7, 1.0 Hz, 2H), 4.05 (s, 6H), 1.27 (d, J=6.2 Hz, 6H).

### Example 116:2-(4,6-dimethoxypyrimidine-5-carboxamido)-N,N-dimethyl-7-(trifluoromethyl)spiro[chromeno[4,3-d]thiazole-4,1′-cyclohexane]-4′-carboxamide

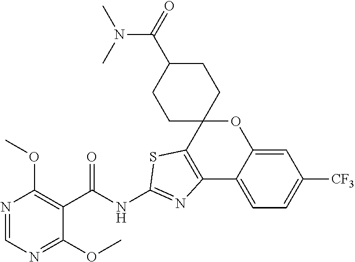 [SMILES: COc1ncnc(OC)c1C(=O)Nc1nc2c(s1)C1(CCC(C(=O)N(C)C)CC1)Oc1cc(C(F)(F)F)ccc1-2]

2-(4,6-dimethoxypyrimidine-5-carboxamido)-7-(trifluoromethyl)spiro[chromeno[4,3-d]thiazole-4,1′-cyclohexane]-4′-carboxylic acid (30 mg, 54.48 μmol, 1 equiv.) and dimethylamine hydrochloride (5.33 mg, 65.39 μmol, 1.2 equiv.) were added to a flame-dried three necked flask. DMF (0.3 mL) was added to the mixture and the reaction was cooled to 0° C. DIEA (24.65 mg, 33.22 μl, 190.73 μmol, 3.5 equiv.) was diluted with DMF (0.1 mL) and added to the mixture at 0° C. HATU (31.1 mg, 81.74 μmol, 1.5 equiv.) was dissolved in DMF (0.1 mL) and added to the mixture at 0° C. The mixture was stirred at 0° C. to rt for 60 minutes. The mixture was quenched with ice-water (10 mL). The aqueous was extracted with EA (20 mL×2). The combine organic layers were washed with brine (20 mL×2), dried over Na₂SO₄, filtrated, and concentrated under reduce pressure to obtain the crude product. The crude product was purified by prep-TLC (EA) then purified by prep-HPLC (0.1% FA water/ACN, 30-100%, 25 minutes) to produce the desire product (8.5 mg, 27%) as white solid. ESI-MS m/z=578.01 [M+H]⁺; Calculated MW: 577.16. ¹H NMR (400 MHz, Chloroform-d) δ 10.78 (s, 1H), 8.37 (s, 1H), 7.62 (d, J=7.9 Hz, 1H), 7.28 (d, J=2.2 Hz, 1H), 7.16 (d, J=7.9 Hz, 1H), 4.03 (s, 6H), 3.09 (s, 3H), 2.98 (s, 3H), 2.63 (t, J=12.1 Hz, 1H), 2.42 (d, J=13.2 Hz, 2H), 2.29-2.13 (m, 2H), 1.77-1.68 (m, 4H).

### Example 117: N—(7-acetyl-4,4-diethyl-4H-chromeno[4,3-d]thiazol-2-yl)-4,6-dimethoxypyrimidine-5-carboxamide

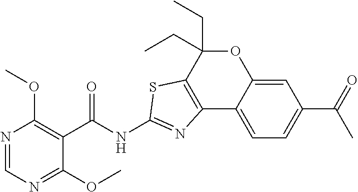 [SMILES: CCC1(CC)Oc2cc(C(C)=O)ccc2-c2nc(NC(=O)c3c(OC)ncnc3OC)sc21]

N—(4,4-diethyl-7-(prop-1-en-2-yl)-4H-chromeno[4,3-d]thiazol-2-yl)-4,6-dimethoxypyrimidine-5-carboxamide (30 mg, 64.30 μmol, 1 equiv.) was added to a flame-dried three necked flask. Lutidine (13.8 mg, 14.87 μl, 128.60 μmol, 2 equiv.) was dissolved in 1,4-dioxane (1.2 mL) and added to the mixture. H₂O (0.6 mL) was added to and the reaction was to 0° C. K₂OsO₄·2H₂O (2.0 mg, 6.43 μmol, 0.1 equiv.) was added to the mixture at 0° C. and the mixture was stirred at 0° C. for 15 minutes. NaIO₄ (55.01 mg, 257.20 μmol, 4 equiv.) was added to the mixture and the mixture was stirred at 0° C. to rt for 3 hours. The mixture was quenched with Na₂S₂O₃ sat. aqueous solution (10 mL). The aqueous phase was extracted with EA (20 mL×2). The combine organic layers were washed with brine (20 mL×2), dried over Na₂SO₄, filtrated, and concentrated under reduce pressure to get the crude product. The crude product was purified by prep-TLC (PE:EA=2:1) to get the desire product (23 mg, 76%) as yellow solid. ESI-MS m/z=468.97. [M+H]⁺; Calculated MW: 468.14. ¹H NMR (400 MHz, Chloroform-d) δ 10.94 (s, 1H), 8.33 (s, 1H), 7.56 (d, J=7.8 Hz, 1H), 7.49-7.41 (m, 2H), 4.02 (s, 6H), 2.57 (s, 3H), 2.11-1.81 (m, 4H), 0.99 (t, J=7.4 Hz, 6H).

### Example 118: N—(4,4-bis((methoxymethoxy)methyl)-7-(trifluoromethyl)-4H-chromeno[4,3-d]thiazol-2-yl)-4,6-dimethoxypyrimidine-5-carboxamide

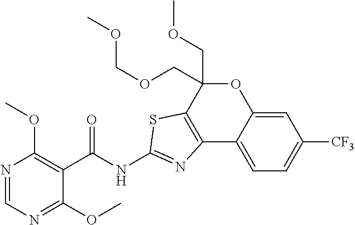 [SMILES: COCOCC1(COC)Oc2cc(C(F)(F)F)ccc2-c2nc(NC(=O)c3c(OC)ncnc3OC)sc21]

To a stirred solution of N—(4,4-bis(hydroxymethyl)-7-(trifluoromethyl)-4H-chromeno[4,3-d]thiazol-2-yl)-4,6-dimethoxypyrimidine-5-carboxamide (4 mg, 0.008 mmol, 1.00 equiv.) in DMSO (0.1 mL) and toluene (0.1 mL) was added dimethoxymethane (0.2 mL) and TsOH·H₂O (0.15 mg, 0.0008 mmol, 1.00 equiv.) at 0° C., and the mixture was stirred overnight at 80° C. The resulting mixture was concentrated under vacuum. The crude product was purified by prep-TLC (PE:EtOAc=1:1) to afford the title compound (1.6 mg, 34%) as a white solid. ESI-MS m/z=586.96 [M+H]⁺; Calculated MW: 586.13. ¹H NMR (400 MHz, Chloroform-d) δ 10.63 (s, 1H), 8.41 (s, 1H), 7.68 (d, J=7.7 Hz, 1H), 7.19 (d, J=8.4 Hz, 2H), 4.67 (s, 4H), 4.06 (s, 6H), 3.96 (d, J=10.3 Hz, 2H), 3.85 (d, J=10.3 Hz, 2H), 3.35 (s, 6H).

### Example 119:4-allyl-N—(4,4-diethyl-7-(trifluoromethyl)-4H-chromeno[4,3-d]thiazol-2-yl)-6-methoxypyrimidine-5-carboxamide

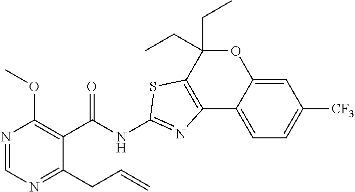 [SMILES: C=CCc1ncnc(OC)c1C(=O)Nc1nc2c(s1)C(CC)(CC)Oc1cc(C(F)(F)F)ccc1-2]

Into a 10 mL round-bottom flask were added 4-chloro-N—(4,4-diethyl-7-(trifluoromethyl)-4H-chromeno[4,3-d]thiazol-2-yl)-6-methoxypyrimidine-5-carboxamide (100.00 mg, 0.20 mmol, 1.00 equiv.), DMF (2.00 mL), H₂O (0.20 mL), potassium allyltrifluoroborate (60.00 mg, 0.40 mmol, 2.00 equiv.), Cs₂CO₃ (196.00 mg, 0.60 mmol, 3.00 equiv.) and Pd(dppf)Cl₂ (16.00 mg, 0.02 mmol, 0.10 equiv.) at room temperature. The resulting mixture was stirred for 16 h at 100° C. under argon. Up completion, the reaction was cooled down to room temperature and diluted with water. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (20.0 mg, 20% yield) as a yellow solid. ESI-MS m/z=505.05 [M+H]⁺; Calculated MW: 504.14; ¹H NMR (400 MHz, Chloroform-d) δ 10.41 (s, 1H), 8.71 (d, J=1.7 Hz, 1H), 7.60 (d, J=8.1 Hz, 1H), 7.17-7.07 (m, 2H), 6.06 (dddd, J=15.3, 10.3, 7.4, 5.6 Hz, 1H), 5.23-5.12 (m, 2H), 4.06 (d, J=1.7 Hz, 3H), 3.79 (dq, J=6.6, 1.7 Hz, 2H), 2.03 (ddd, J=14.4, 7.3, 1.7 Hz, 2H), 1.97-1.85 (m, 2H), 0.99 (td, J=7.4, 1.7 Hz, 6H).

### Example 120: N—(4,4-diethyl-7-(trifluoromethyl)-4H-chromeno[4,3-d]thiazol-2-yl)-4-methoxy-6-(methylamino)pyrimidine-5-carboxamide

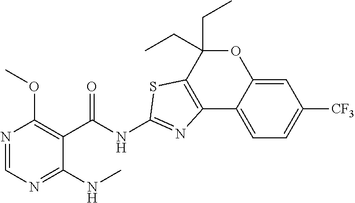 [SMILES: CCC1(CC)Oc2cc(C(F)(F)F)ccc2-c2nc(NC(=O)c3c(NC)ncnc3OC)sc21]

To a stirred solution of 4-chloro-N—(4,4-diethyl-7-(trifluoromethyl)-4H-chromeno[4,3-d]thiazol-2-yl)-6-methoxypyrimidine-5-carboxamide (10.0 mg, 0.020 mmol, 1.00 equiv.) in DMF (1.00 mL) was added TEA (20.0 mg, 0.200 mmol, 10.00 equiv.) and NH₂CH₃·HCl (4.00 mg, 0.060 mmol, 3.00 equiv.) at room temperature. The resulting mixture was stirred for 16 h at 40° C. under argon. The reaction was diluted with water, extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (8.0 mg, 81% yield) as a white solid. ESI-MS m/z=494.02 [M+H]⁺; Calculated MW: 493.14; ¹H NMR (400 MHz, Chloroform-d) δ 11.06 (s, 1H), 9.43 (d, J=5.3 Hz, 1H), 8.34 (s, 1H), 7.77 (d, J=7.9 Hz, 1H), 7.20-7.16 (m, 1H), 7.12 (d, J=1.6 Hz, 1H), 4.23 (s, 3H), 3.10 (d, J=4.9 Hz, 3H), 2.08-1.96 (m, 2H), 1.89 (dq, J=14.5, 7.3 Hz, 2H), 0.97 (t, J=7.4 Hz, 6H).

### Example 121: N—(4,4-diethyl-7-(trifluoromethyl)-4H-chromeno[4,3-d]thiazol-2-yl)-4-hydroxy-6-methoxypyrimidine-5-carboxamide

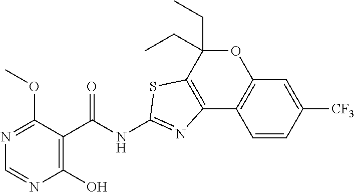 [SMILES: CCC1(CC)Oc2cc(C(F)(F)F)ccc2-c2nc(NC(=O)c3c(O)ncnc3OC)sc21]

To a stirred solution of 4-chloro-N—(4,4-diethyl-7-(trifluoromethyl)-4H-chromeno[4,3-d]thiazol-2-yl)-6-methoxypyrimidine-5-carboxamide (10.0 mg, 0.020 mmol, 1.00 equiv.) in THF (1.00 mL) was added NaOH (4.0 mg, 0.100 mmol, 5.00 equiv.) in H₂O (0.5 mL) at room temperature. The resulting mixture was stirred for 16 h at 60° C. under argon atmosphere. The reaction was diluted with water, extracted with EtOAc (3×15 mL). The combined organic layers were washed with brine (2×10 mL) and dried over anhydrous Na₂SO₄. After filtration, the filtrate was concentrated under reduced pressure. The residue was purified by prep-TLC (DCM/MeOH (10:1)) to afford the title compound (2.0 mg, 20% yield) as a white solid. ESI-MS m/z=481.02 [M+H]⁺; Calculated MW: 480.11; ¹H NMR (400 MHz, Chloroform-d) δ 12.39 (d, J=110.1 Hz, 2H), 8.36 (s, 1H), 7.76 (d, J=7.8 Hz, 1H), 7.19-7.10 (m, 2H), 4.24 (s, 3H), 2.00 (dt, J=14.7, 7.3 Hz, 2H), 1.89 (dq, J=14.7, 7.4 Hz, 2H), 0.96 (t, J=7.3 Hz, 6H).

### Example 122: N—(4,4-diethyl-7-(trifluoromethyl)-4H-chromeno[4,3-d]thiazol-2-yl)-4-methoxy-6-(methoxymethoxy)pyrimidine-5-carboxamide

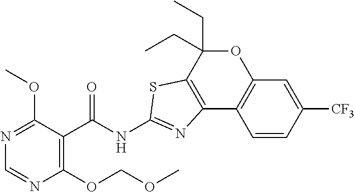 [SMILES: CCC1(CC)Oc2cc(C(F)(F)F)ccc2-c2nc(NC(=O)c3c(OC)ncnc3OCOC)sc21]

To a stirred solution of N—(4,4-diethyl-7-(trifluoromethyl)-4H-chromeno[4,3-d]thiazol-2-yl)-4-hydroxy-6-methoxypyrimidine-5-carboxamide (40.0 mg, 0.083 mmol, 1.00 equiv.) in toluene (2.00 mL) and DMSO (0.50 mL) was added TsOH (14.0 mg, 0.083 mmol, 1.00 equiv.) and dimethoxymethane (633.00 mg, 8.300 mmol, 100.00 equiv.) at room temperature. The resulting mixture was stirred for 16 h at 100° C. under argon. The reaction was diluted with water, extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (DCM/MeOH (10:1)) to afford the title compound (1.4 mg, 3.3% yield) as a white solid. ESI-MS m/z=525.00 [M+H]⁺; Calculated MW: 524.13; ¹H NMR (400 MHz, Chloroform-d) δ 12.76 (s, 1H), 8.26 (s, 1H), 7.76 (d, J=7.9 Hz, 1H), 7.19-7.14 (m, 1H), 7.09 (d, J=1.7 Hz, 1H), 5.42 (s, 2H), 4.19 (s, 3H), 3.51 (s, 3H), 1.99 (td, J=14.7, 14.1, 6.8 Hz, 2H), 1.87 (dq, J=14.6, 7.4 Hz, 2H), 0.95 (t, J=7.4 Hz, 6H).

### Example 123: isopropyl 2-(4,6-dimethoxypyrimidine-5-carboxamido)-7-(trifluoromethyl)spiro[chromeno[4,3-d]thiazole-4,1′-cyclohexane]-4′-carboxylate

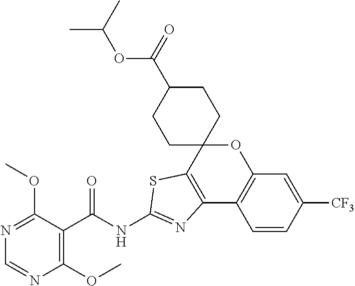 [SMILES: COc1ncnc(OC)c1C(=O)Nc1nc2c(s1)C1(CCC(C(=O)OC(C)C)CC1)Oc1cc(C(F)(F)F)ccc1-2]

2-(4,6-dimethoxypyrimidine-5-carboxamido)-7-(trifluoromethyl)spiro[chromeno[4,3-d]thiazole-4,1′-cyclohexane]-4′-carboxylic acid (20 mg, 36.33 μmol, 1 equiv.) was added to a flame-dried three necked flask. DCM (0.3 mL) was added to the mixture and the reaction was cooled to 0° C. (COCl)₂ (13.8 mg, 9.22 μl, 108.99 μmol, 3.0 equiv.) was diluted with DCM (0.1 mL) and added to the mixture at 0° C. DMF (0.27 mg, 3.63 μmol, 0.1 equiv.) was diluted with DCM (0.1 mL) and added to the mixture at 0° C. The mixture was stirred at 0° C. for 30 minutes, and was added to the solution of propan-2-ol (2 mL) dropwise at 0° C. The mixture was stirred at 0° C. for 10 minutes and concentrated under reduce pressure. The residue was quenched with ice-water (10 mL). The aqueous phase was extracted with EA (20 mL×2). The combine organic layers were washed with brine (20 mL×2), dried over Na₂SO₄, filtrated, and concentrated under reduce pressure to get the crude product. The crude product was purified by prep-TLC (PE:EA=1:1) to get the desire product (13.8 mg, 64%) as white solid. ESI-MS m/z=593.06 [M+H]⁺; Calculated MW: 592.16. ¹H NMR (400 MHz, Chloroform-d) δ 11.08 (s, 1H), 8.30 (s, 1H), 7.56 (d, J=7.9 Hz, 1H), 7.24-7.19 (m, 1H), 7.16-7.09 (m, 1H), 5.05 (hept, J=6.2 Hz, 1H), 3.97 (s, 6H), 2.43-2.32 (m, 3H), 2.10-1.88 (m, 4H), 1.70 (td, J=13.6, 4.3 Hz, 2H), 1.26 (d, J=6.3 Hz, 6H).

### Example 124: N—(4,4-diethyl-7-methyl-4H-chromeno[4,3-d]thiazol-2-yl)-4,6-dimethoxypyrimidine-5-carboxamide

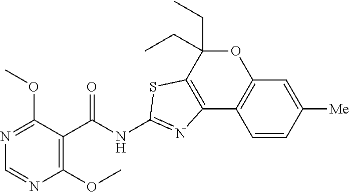 [SMILES: CCC1(CC)Oc2cc(C)ccc2-c2nc(NC(=O)c3c(OC)ncnc3OC)sc21]

Into a 10 mL round-bottom flask were added N—(7-bromo-4,4-diethyl-4H-chromeno[4,3-d]thiazol-2-yl)-4,6-dimethoxypyrimidine-5-carboxamide (50.0 mg, 0.099 mmol, 1.00 equiv.), DMF (1.00 mL), H₂O (0.20 mL), methylboronic acid (30.0 mg, 0.50 mmol, 5.00 equiv.), K₂CO₃ (34.0 mg, 0.25 mmol, 2.5 equiv.) and Pd(PPh₃)₄ (11.0 mg, 0.009 mmol, 0.10 equiv.) at room temperature. The resulting mixture was stirred for 4 h at 100° C. under argon atmosphere. The reaction was cooled down to room temperature and diluted with water. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (4.0 mg, 9% yield) as a yellow solid. ESI-MS m/z=441.06 [M+H]⁺; Calculated MW: 440.15; ¹H NMR (400 MHz, Chloroform-d) δ 11.78 (s, 1H), 8.19 (d, J=1.3 Hz, 1H), 7.23 (dd, J=7.7, 1.2 Hz, 1H), 6.68 (s, 1H), 6.60-6.55 (m, 1H), 3.89 (d, J=1.2 Hz, 6H), 2.29 (d, J=3.2 Hz, 3H), 2.04-1.93 (m, 2H), 1.87 (dtd, J=15.6, 8.2, 7.8, 6.5 Hz, 2H), 0.99 (td, J=7.5, 1.3 Hz, 6H).

### Example 125:2-(4,6-dimethoxypyrimidine-5-carboxamido)-N-isopropyl-7-(trifluoromethyl)spiro[chromeno[4,3-d]thiazole-4,1′-cyclohexane]-4′-carboxamide

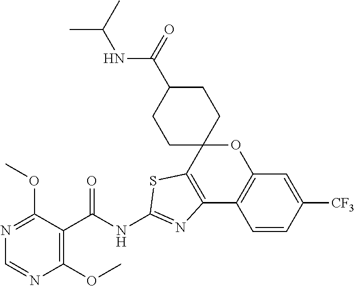 [SMILES: COc1ncnc(OC)c1C(=O)Nc1nc2c(s1)C1(CCC(C(=O)NC(C)C)CC1)Oc1cc(C(F)(F)F)ccc1-2]

2-(4,6-dimethoxypyrimidine-5-carboxamido)-7-(trifluoromethyl)spiro[chromeno[4,3-d]thiazole-4,1′-cyclohexane]-4′-carboxylic acid (10 mg, 18.17 μmol, 1 equiv.) and propan-2-amine (1.29 mg, 1.87 μl, 21.80 μmol, 1.2 equiv.) were added to a flame-dried three necked flask. DMF (0.1 mL) was added to the mixture and the reaction was cooled to 0° C. DIEA (5.9 mg, 7.91 μl, 45.41 μmol, 2.5 equiv.) was diluted with DMF (0.1 mL) and added to the mixture at 0° C. HATU (10.4 mg, 27.25 μmol, 1.5 equiv.) was dissolved in DMF (0.1 mL) and added to the mixture at 0° C. The mixture was stirred at 0° C. to rt for 60 minutes and quenched with ice-water (10 mL). The aqueous was extracted with EA (20 mL×2). The combine organic layers were washed with brine (20 mL×2), dried over Na₂SO₄, filtrated, and concentrated under reduce pressure to produce the crude product. The crude product was purified by prep-TLC (PE:EA=1:2) to generate the desire product (1.2 mg, 11%) as white solid. ESI-MS m/z=592.16 [M+H]⁺; Calculated MW: 591.17. ¹H NMR (400 MHz, Chloroform-d) δ 10.89 (s, 1H), 8.34 (s, 1H), 7.60 (d, J=7.9 Hz, 1H), 7.30-7.26 (m, 1H), 7.15 (dt, J=7.9, 1.1 Hz, 1H), 5.30 (d, J=8.0 Hz, 1H), 4.20-4.05 (m, 1H), 4.00 (s, 6H), 2.41 (d, J=13.9 Hz, 2H), 2.18-2.00 (m, 3H), 1.83 (d, J=11.9 Hz, 2H), 1.69 (t, J=13.3 Hz, 2H), 1.17 (d, J=6.5 Hz, 6H).

### Example 126: N-benzyl-2-(4,6-dimethoxypyrimidine-5-carboxamido)-N-methyl-7-(trifluoromethyl)spiro[chromeno[4,3-d]thiazole-4,1′-cyclohexane]-4′-carboxamide

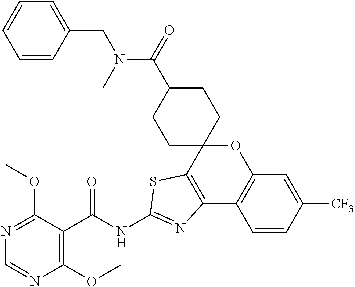 [SMILES: COc1ncnc(OC)c1C(=O)Nc1nc2c(s1)C1(CCC(C(=O)N(C)Cc3ccccc3)CC1)Oc1cc(C(F)(F)F)ccc1-2]

2-(4,6-dimethoxypyrimidine-5-carboxamido)-7-(trifluoromethyl)spiro[chromeno[4,3-d]thiazole-4,1′-cyclohexane]-4′-carboxylic acid (20 mg, 36.33 μmol, 1 equiv.) and N-methyl-1-phenylmethanamine (5.28 mg, 1.87 μl, 43.60 μmol, 1.2 equiv.) were added to a flame-dried three necked flask. DMF (0.2 mL) was added to the mixture and the reaction was cooled to 0° C. DIEA (11.7 mg, 15.82 μl, 90.83 μmol, 2.5 equiv.) was diluted with DMF (0.1 mL) and added to the mixture at 0° C. HATU (20.7 mg, 54.50 μmol, 1.5 equiv.) was dissolved in DMF (0.1 mL) and added to the mixture at 0° C. The mixture was stirred at rt for 60 minutes. The mixture was quenched with ice-water (10 mL). The aqueous phase was extracted with EA (20 mL×2) for twice. The combine organic layers were washed with brine (20 mL×2), dried over Na₂SO₄, filtrated, and concentrated under reduce pressure to get the crude product. The crude product was purified by prep-TLC (PE:EA=1:2) to get the desire product (4r,4′r)-N-benzyl-2-(4,6-dimethoxypyrimidine-5-carboxamido)-N-methyl-7-(trifluoromethyl)spiro[chromeno[4,3-d]thiazole-4,1′-cyclohexane]-4′-carboxamide (10 mg, 42%) as white solid. ESI-MS m/z-654.16 [M+H]⁺; Calculated MW: 653.19. ¹H NMR (400 MHz, Chloroform-d) § 11.01 (s, 1H), 8.31 (dd, J=5.4, 1.4 Hz, 1H), 7.57 (t, J=8.4 Hz, 1H), 7.44-7.26 (m, 4H), 7.25-7.02 (m, 3H), 4.62 (d, J=6.0 Hz, 2H), 3.97 (dd, J=6.5, 1.4 Hz, 6H), 2.99 (dd, J=6.0, 1.4 Hz, 3H), 2.73-2.59 (m, 1H), 2.34 (dq, J=50.9, 12.7, 11.8 Hz, 4H), 1.83-1.62 (m, 4H).

### Example 127:2-(4,6-dimethoxypyrimidine-5-carboxamido)-N-isopropyl-N-methyl-7-(trifluoromethyl)spiro[chromeno[4,3-d]thiazole-4,1′-cyclohexane]-4′-carboxamide

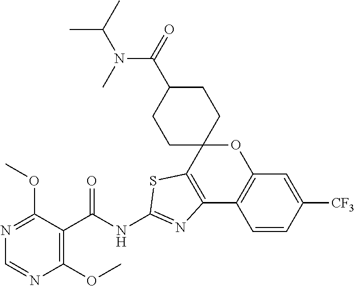 [SMILES: COc1ncnc(OC)c1C(=O)Nc1nc2c(s1)C1(CCC(C(=O)N(C)C(C)C)CC1)Oc1cc(C(F)(F)F)ccc1-2]

2-(4,6-dimethoxypyrimidine-5-carboxamido)-7-(trifluoromethyl)spiro[chromeno[4,3-d]thiazole-4,1′-cyclohexane]-4′-carboxylic acid (10 mg, 18.17 μmol, 1 equiv.) and N-methylpropan-2-amine (1.59 mg, 21.80 μl, 1.2 equiv.) were added to a flame-dried three necked flask. DMF (0.1 mL) was added to the mixture and the reaction was cooled to 0° C. DIEA (5.9 mg, 8.11 μl, 45.41 μmol, 2.5 equiv.) was diluted with DMF (0.1 mL) and added to the mixture at 0° C. HATU (10.4 mg, 27.25 μmol, 1.5 equiv.) was dissolved in DMF (0.1 mL) and added to the mixture at 0° C. The mixture was stirred at rt for 60 minutes. The mixture was quenched with ice-water (10 mL). The aqueous was extracted with EA (20 mL×2). The combine organic layers were washed with brine (20 mL×2), dried over Na₂SO₄, filtrated, and concentrated under reduce pressure to get the crude product. The crude product was purified by prep-TLC (PE:EA=1:2) to get the desire product (4 mg, 36%) as white solid. ESI-MS m/z=606.13 [M+H]⁺; Calculated MW: 605.19. ¹H NMR (400 MHz, Chloroform-d) δ 11.08 (s, 1H), 8.31 (s, 1H), 7.56 (d, J=7.9 Hz, 1H), 7.28 (d, J=2.1 Hz, 1H), 7.13 (dd, J=7.9, 1.7 Hz, 1H), 4.96-4.89 (m, 0.5H), 4.27-4.09 (m, 0.5H), 3.97 (s, 6H), 2.89 (s, 1.5H), 2.80 (s, 1.5H), 2.60 (dt, J=24.5, 12.0 Hz, 1H), 2.41 (d, J=13.3 Hz, 2H), 2.34-2.14 (m, 2H), 1.73 (dd, J=19.2, 8.5 Hz, 4H), 1.23 (d, J=6.5 Hz, 3H), 1.10 (d, J=6.9 Hz, 3H).

### Example 128: ethyl (4s,4's)-7-bromo-2-(4,6-dimethoxypyrimidine-5-carboxamido)spiro[chromeno[4,3-d]thiazole-4,1′-cyclohexane]-4′-carboxylate

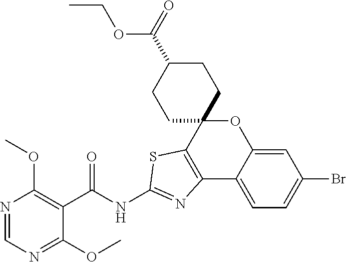

### Example 128A: ethyl(2s,4's)-7-bromo-4-oxospiro[chromane-2,1′-cyclohexane]-4′-carboxylate

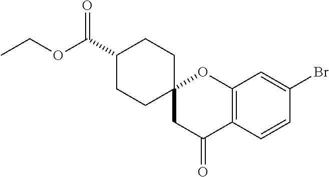

To a stirred solution of 1-(4-bromo-2-hydroxyphenyl)ethan-1-one (1.0 g, 4.65 mmol, 1.0 equiv.) in ACN (10 mL) was added ethyl 4-oxocyclohexane-1-carboxylate (949.8 mg, 0.889 mL, 5.58 mmol, 1.2 equiv.) and pyrrolidine (661.5 mg, 0.776 mL, 9.30 mmol, 2.0 equiv.) at 0° C. The mixture was stirred at 40° C. for 16 hours. Then the mixture was concentrated under vacuum. The resulting residue was extracted with ethyl acetate (2×20 mL). The combined organic layers were washed with brine (2×30 mL), dried over anhydrous Na₂SO₄. The resulting mixture was concentrated under reduce pressure. The crude product was purified by silica gel column chromatography (PE:EA=60:1 to 15:1) to afford the title compound (660 mg, 39%) as yellow solid, R_{f}=0.6 on the TLC (PE:EA=5:1). ¹H NMR (400 MHz, Chloroform-d) δ 7.68 (dd, J=8.4, 0.8 Hz, 1H), 7.16 (t, J=1.3 Hz, 1H), 7.13-7.05 (m, 1H), 4.22-4.04 (m, 2H), 2.74 (s, 2H), 2.60-2.49 (m, 1H), 2.08-1.91 (m, 2H), 1.86-1.72 (m, 6H), 1.25 (td, J=7.1, 0.8 Hz, 3H).

### Example 128B: ethyl (4s,4's)-2-amino-7-bromospiro[chromeno[4,3-d]thiazole-4,1′-cyclohexane]-4′-carboxylate

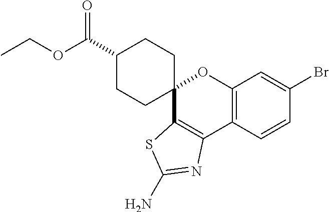

To a stirred solution of ethyl(2s,4's)-7-bromo-4-oxospiro[chromane-2,1′-cyclohexane]-4′-carboxylate (660 mg, 1.8 mmol, 1.0equiv.) in toluene (10 mL) was added TsOH·H₂O (68.4 mg, 0.359 mmol, 0.2 equiv.) and pyrrolidine (639.1 mg, 0.750 mL, 8.99 mmol, 5.0 equiv.) at room temperature. The reaction mixture was stirred at 120° C. for 2 hours. Then it was cooled to room temperature and concentrated. The resulting mixture was dissolved in methanol (7 mL) and sulfur (114.05 mg, 4.49 mmol, 2.5 equiv.) was added. The mixture was stirred for 10 minutes at room temperature then cyanamide (377.8 mg, 4.49 mmol, 0.295 mL, 2.5equiv.) was added. The mixture was stirred overnight at room temperature. After concentration, the resulting residue was extracted with ethyl acetate (2×20 mL). The combined organic layers were washed with brine (2×30 mL), dried over anhydrous Na₂SO₄. The resulting mixture was concentrated under vacuum. The crude product was purified by silica gel column chromatography (PE:EA=5:3) to afford the title compound (23 mg, 3%) as a yellow solid. ¹H NMR (400 MHz, Chloroform-d) δ 7.47-7.40 (m, 1H), 7.12-7.04 (m, 2H), 5.06 (s, 2H), 4.16 (q, J=7.1 Hz, 2H), 2.63 (q, J=4.8 Hz, 1H), 2.12-1.99 (m, 4H), 1.97-1.84 (m, 4H), 1.27 (t, J=7.1 Hz, 3H).

### Example 128C: ethyl (4s,4's)-7-bromo-2-(4,6-dimethoxypyrimidine-5-carboxamido)spiro[chromeno[4,3-d]thiazole-4,1′-cyclohexane]-4′-carboxylate

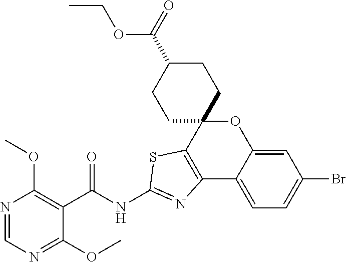

Ethyl (4s,4's)-2-amino-7-bromospiro[chromeno[4,3-d]thiazole-4,1′-cyclohexane]-4′-carboxylate (10 mg, 23.62 μmol, 1 equiv.) and 4,6-dimethoxypyrimidine-5-carboxylic acid (6.53 mg, 35.43 μmol, 1.5 equiv.) were added to a flame-dried three necked flask. ACN (0.2 mL) was added to the mixture and the reaction was cooled to 0° C. NMI (6.8 mg, 6.53 μl, 82.68 μmol, 3.5 equiv.) was added to the mixture at 0° C. TCFH (9.9 mg, 35.43 μmol, 1.5 equiv.) was dissolved in 0.1 mL ACN and added to the mixture at 0° C. The mixture was stirred at rt for 3 hours then stirred at 60° C. for 16 hours. The mixture was concentrated under reduce pressure then quenched with ice-water (20 mL). The aqueous phase was extracted with EA (20 mL×2). The combine organic layers were washed with brine (20 mL×2), dried over Na₂SO₄, filtrated and concentrated under reduce pressure. The crude product was purified by silica gel column chromatography, eluted with PE:EA:DCM=1:1:1 to afford the title compound (3 mg, 22%) as yellow solid. ESI-MS m/z=588.96 [M+H]⁺; Calculated MW: 588.06 ¹H NMR (400 MHz, Chloroform-d) δ 10.98 (s, 1H), 8.33 (d, J=1.2 Hz, 1H), 7.36 (dd, J=8.1, 1.2 Hz, 1H), 7.11 (d, J=1.8 Hz, 1H), 7.02 (dt, J=8.2, 1.6 Hz, 1H), 4.25-4.13 (m, 2H), 3.98 (d, J=1.3 Hz, 6H), 2.66 (d, J=5.1 Hz, 1H), 2.14-2.04 (m, 4H), 2.04-1.90 (m, 4H), 1.29 (td, J=7.1, 1.2 Hz, 3H).

### Example 129: ethyl (4r,4′r)-7-bromo-2-(4,6-dimethoxypyrimidine-5-carboxamido)spiro[chromeno[4,3-d]thiazole-4,1′-cyclohexane]-4′-carboxylate

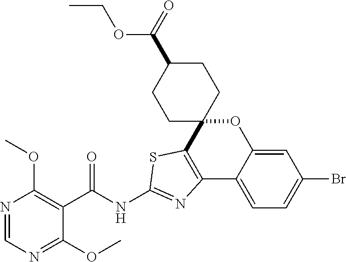

### Example 129A: ethyl(2r,4′r)-7-bromo-4-oxospiro[chromane-2,1′-cyclohexane]-4′-carboxylate

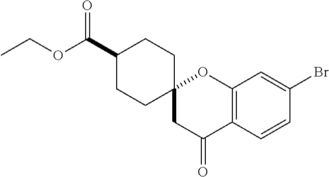

To a stirred solution of 1-(4-bromo-2-hydroxyphenyl)ethan-1-one (1.0 g, 4.65 mmol, 1.0 equiv.) in ACN (10 mL) was added ethyl 4-oxocyclohexane-1-carboxylate (949.8 mg, 0.889 mL, 5.58 mmol, 1.2equiv.) and pyrrolidine (661.5 mg, 0.776 mL, 9.30 mmol, 2.0 equiv.) at 0° C. The mixture was stirred at 40° C. for 16 hours. Then the mixture was concentrated under vacuum. The resulting residue was extracted with ethyl acetate (2×20 mL). The combined organic layers were washed with brine (2×30 mL), dried over anhydrous Na₂SO₄. The resulting mixture was concentrated under vacuum. The crude product was purified by silica gel column chromatography (PE:EA=60:1 to 15:1) to afford the title compound (1.15 g, 67%) as yellow solid, R_{f}=0.55 on the TLC (PE:EA=5:1). ¹H NMR (400 MHz, Chloroform-d) δ 7.69 (dd, J=8.5, 1.0 Hz, 1H), 7.22 (d, J=1.7 Hz, 1H), 7.11 (dt, J=8.5, 1.6 Hz, 1H), 4.14 (qd, J=7.2, 1.1 Hz, 2H), 2.66 (d, J=1.1 Hz, 2H), 2.30 (tt, J=10.4, 5.5 Hz, 1H), 2.21-2.11 (m, 2H), 1.91-1.80 (m, 4H), 1.51-1.35 (m, 2H), 1.25 (td, J=7.1, 1.1 Hz, 3H).

### Example 129B: ethyl (4r,4′r)-2-amino-7-bromospiro[chromeno[4,3-d]thiazole-4,1′-cyclohexane]-4′-carboxylate

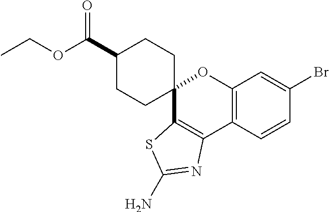

To a stirred solution of ethyl(2r,4′r)-7-bromo-4-oxospiro[chromane-2,1′-cyclohexane]-4′-carboxylate (1.95 g, 5.31 mmol, 1.0 equiv.) in toluene (20 mL) was added TsOH·H₂O (202 mg, 1.06 mmol, 0.2 equiv.) and pyrrolidine (1.89 g, 2.22 mL, 26.55 mmol, 5.0 equiv.) at room temperature. The reaction mixture was stirred at 120° C. for 2 hours. Then it was cooled to room temperature and concentrated. The resulting mixture was dissolved in methanol (20 mL) and sulfur (425.59 mg, 13.27 mmol, 2.5 equiv.) was added. The mixture was stirred for 10 minutes at room temperature. Then cyanamide (1.12 g, 0.872 mL, 13.27 mmol, 2.5equiv.) was added. The mixture was stirred overnight at room temperature. After concentration, the resulting residue was extracted with ethyl acetate (2×20 mL). The combined organic layers were washed with brine (2×30 mL), dried over anhydrous Na₂SO₄. The resulting mixture was concentrated under vacuum. The crude product was purified by silica gel column chromatography (PE:EA=5:3) to afford the title compound (218 mg, 9.7%) as yellow solid. ¹H NMR (400 MHz, Chloroform-d) δ 7.41 (dd, J=8.2, 1.8 Hz, 1H), 7.13 (d, J=2.0 Hz, 1H), 7.09 (dt, J=8.1, 2.0 Hz, 1H), 5.43 (s, 2H), 4.15 (qd, J=7.1, 1.8 Hz, 2H), 2.32 (d, J=13.6 Hz, 3H), 2.06-1.93 (m, 2H), 1.87 (d, J=13.4 Hz, 2H), 1.60-1.49 (m, 2H), 1.27 (td, J=7.1, 1.9 Hz, 3H).

### Example 129C: ethyl (4r,4′r)-7-bromo-2-(4,6-dimethoxypyrimidine-5-carboxamido)spiro[chromeno[4,3-d]thiazole-4,1′-cyclohexane]-4′-carboxylate

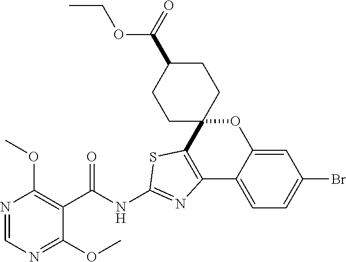

Ethyl (4r,4′r)-2-amino-7-bromospiro[chromeno[4,3-d]thiazole-4,1′-cyclohexane]-4′-carboxylate (247 mg, 583.48 μmol, 1 equiv.) and 4,6-dimethoxypyrimidine-5-carboxylic acid (161.17 mg, 875.21 μmol, 1.5 equiv.) were added to a flame-dried three necked flask. ACN (3 mL) was added and the mixture was cooled to 0° C. NMI (167.67 mg, 161 μl, 2040 μmol, 3.5 equiv.) was added to the mixture at 0° C. TCFH (245.57 mg, 875.21 μmol, 1.5 equiv.) was dissolved in 2 mL ACN and added to the mixture at 0° C. The mixture was stirred at rt for 3 hours then stirred at 60° C. for 16 hours. The mixture was concentrated under reduce pressure then quenched with ice-water (20 mL). The aqueous phase was extracted with EA (20 mL×2). The combine organic layers were washed with brine (20 mL×2), dried over Na₂SO₄, filtered, and concentrated under reduce pressure. The crude product was purified by silica gel column chromatography, eluted with PE:EA:DCM=1:1:1 to afford the title compound (170 mg, 35%) as yellow solid. ¹H NMR (400 MHz, Chloroform-d) δ 8.34 (d, J=4.0 Hz, 1H), 7.36 (d, J=8.1 Hz, 1H), 7.16 (d, J=1.8 Hz, 1H), 7.03 (dd, J=8.2, 1.9 Hz, 1H), 4.18 (p, J=7.0 Hz, 2H), 3.99 (d, J=3.0 Hz, 6H), 2.36 (d, J=12.4 Hz, 3H), 2.08-1.90 (m, 4H), 1.72-1.63 (m, 2H), 1.33-1.27 (m, 3H).

### Example 130: ethyl (4r,4′r)-2-(4,6-dimethoxypyrimidine-5-carboxamido)-7-isopropylspiro[chromeno[4,3-d]thiazole-4,1′-cyclohexane]-4′-carboxylate

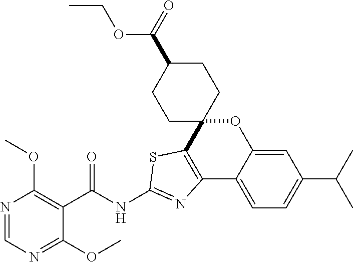

### Example 130A: ethyl (4r,4′r)-2-(4,6-dimethoxypyrimidine-5-carboxamido)-7-(prop-1-en-2-yl)spiro[chromeno[4,3-d]thiazole-4,1′-cyclohexane]-4′-carboxylate

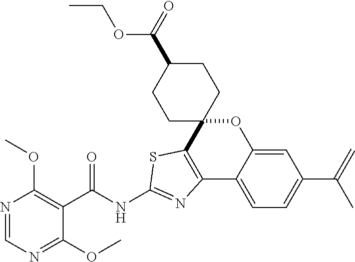

Ethyl (4r,4′r)-7-bromo-2-(4,6-dimethoxypyrimidine-5-carboxamido)spiro[chromeno[4,3-d]thiazole-4,1′-cyclohexane]-4′-carboxylate (20 mg, 33.93 μmol, 1 equiv.), 4,4,5,5-tetramethyl-2-(prop-1-en-2-yl)-1,3,2-dioxaborolane (28.5 mg, 169.65 μmol, 5 equiv.), K₂CO₃ (14.1 mg, 101.79 μmol, 3.0 equiv.) and Pd(dppf)Cl₂ (2.8 mg, 3.39 μmol, 0.1 equiv.) were added to a flame-dried tube. The tube was evacuated and back-filled with Ar. Dioxane (0.4 mL) and H₂O (0.1 mL) were added to the mixture. The mixture was stirred at 80° C. for 16 hours. The mixture was concentrated under reduce pressure then quenched with ice-water (5 mL). The aqueous was extracted with EA (10 mL×2). The combined organic layers were washed with brine (20 mL×2), dried over Na₂SO₄, filtrated and concentrated under reduce pressure. The crude product was purified by prep-TLC (PE:EA=2:1) to afford the title compound (18 mg, 96%) as yellow solid. ¹H NMR (400 MHz, Chloroform-d) δ 10.83 (s, 1H), 8.35 (d, J=1.0 Hz, 1H), 7.46 (d, J=7.8 Hz, 1H), 7.09 (t, J=1.3 Hz, 1H), 7.05 (dd, J=8.0, 1.6 Hz, 1H), 5.42 (s, 1H), 5.09 (d, J=1.5 Hz, 1H), 4.17 (q, J=7.2 Hz, 2H), 4.00 (d, J=1.0 Hz, 6H), 2.39 (d, J=13.3 Hz, 3H), 2.15 (s, 3H), 2.09-1.96 (m, 2H), 1.96-1.92 (m, 3H), 1.75-1.63 (m, 1H), 1.32-1.26 (m, 3H).

### Example 130B: ethyl (4r,4′r)-2-(4,6-dimethoxypyrimidine-5-carboxamido)-7-isopropylspiro[chromeno[4,3-d]thiazole-4,1′-cyclohexane]-4′-carboxylate

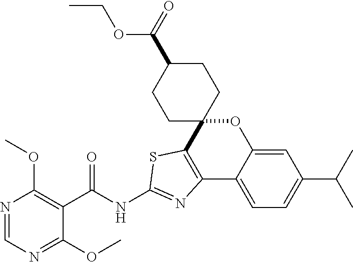

Ethyl (4r,4′r)-2-(4,6-dimethoxypyrimidine-5-carboxamido)-7-(prop-1-en-2-yl)spiro[chromeno[4,3-d]thiazole-4,1′-cyclohexane]-4′-carboxylate (18 mg, 32.69 μmol, 1 equiv.) was added to a flame-dried three necked flask. MeOH (0.4 mL) and Pd/C (10%, 20 mg) were added to the mixture. The mixture was evacuated and back-filled with H₂ for 3 times then stirred at rt for 16 hours. The mixture was filtered and concentrated under reduce pressure. The crude product was purified by prep-TLC (PE:EA=2:1) then purified by prep-HPLC (0.1% FA, 20% to 90% CH₃CN/H₂O, 30 minutes) to produce the desire product ethyl (4r,4′r)-2-(4,6-dimethoxypyrimidine-5-carboxamido)-7-isopropylspiro[chromeno[4,3-d]thiazole-4,1′-cyclohexane]-4′-carboxylate (3 mg, 17%) as white solid. ESI-MS m/z=553.16 [M+H]⁺; Calculated MW: 552.20. ¹H NMR (400 MHz, Chloroform-d) δ 11.42 (s, 1H), 8.21 (s, 1H), 7.31 (d, J=7.8 Hz, 1H), 6.84 (d, J=1.6 Hz, 1H), 6.70 (dd, J=7.9, 1.7 Hz, 1H), 4.17 (q, J=7.1 Hz, 2H), 3.91 (s, 6H), 2.84 (p, J=6.9 Hz, 1H), 2.40-2.33 (m, 3H), 2.13-2.03 (m, 2H), 1.95-1.85 (m, 2H), 1.73-1.63 (m, 2H), 1.29 (t, J=7.1 Hz, 3H), 1.25 (d, J=6.9 Hz, 6H).

### Example 131: tert-butyl(2-(4,6-dimethoxypyrimidine-5-carboxamido)-7-(trifluoromethyl)spiro[chromeno[4,3-d]thiazole-4,1′-cyclobutan]-3′-yl)carbamate

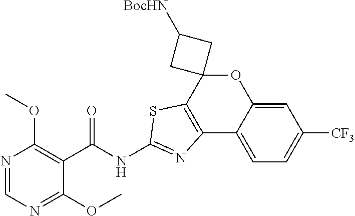 [SMILES: COc1ncnc(OC)c1C(=O)Nc1nc2c(s1)C1(CC(NC(=O)OC(C)(C)C)C1)Oc1cc(C(F)(F)F)ccc1-2]

### Example 131A: tert-butyl (4-oxo-7-(trifluoromethyl)spiro[chromane-2,1′-cyclobutan]-3′-yl)carbamate

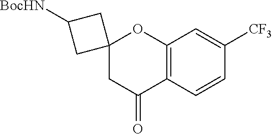 [SMILES: CC(C)(C)OC(=O)NC1CC2(CC(=O)c3ccc(C(F)(F)F)cc3O2)C1]

To a stirred solution of 1-(2-hydroxy-4-(trifluoromethyl)phenyl)ethan-1-one (300 mg, 1.47 mmol, 1.0 equiv.) in MeOH (3 mL) was added tert-butyl (3-oxocyclobutyl)carbamate (407 mg, 2.20 mmol, 2.0equiv.) and pyrrolidine (708 mg, 2.94 mmol, 2.0 equiv.) at 0° C. The reaction mixture was stirred overnight at room temperature. After concentration, the resulting residue was diluted with water, extracted with ethyl acetate (3×80 mL). The combined organic layers were washed with brine, dried over anhydrous Na₂SO₄. The resulting mixture was concentrated under vacuum. The crude product was purified by silica gel column chromatography (PE:EA=10:1) to afford the title compound (330 mg, 60%) as a yellow solid. ¹H NMR (400 MHz, Chloroform-d) δ 7.94 (dt, J=8.5, 1.6 Hz, 1H), 7.27 (d, J=8.5 Hz, 1H), 7.23 (d, J=1.6 Hz, 1H), 4.72 (s, 1H), 3.94 (s, 1H), 2.91 (d, J=13.5 Hz, 2H), 2.75-2.64 (m, 2H), 2.27-2.17 (m, 1H), 2.14 (d, J=11.4 Hz, 1H), 1.42 (s, 9H).

### Example 131B: tert-butyl(2-amino-7-(trifluoromethyl)spiro[chromeno[4,3-d]thiazole-4,1′-cyclobutan]-3′-yl)carbamate

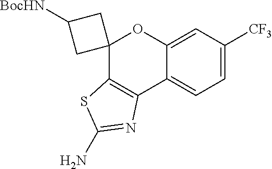 [SMILES: CC(C)(C)OC(=O)NC1CC2(C1)Oc1cc(C(F)(F)F)ccc1-c1nc(N)sc12]

To a stirred solution of tert-butyl (4-oxo-7-(trifluoromethyl)spiro[chromane-2,1′-cyclobutan]-3′-yl)carbamate (400 mg, 1.08 mmol, 1.0 equiv.) in n-hexane (8 mL) was added p-TsOH (41.8 mg, 0.22 mmol, 0.1 equiv.) and pyrrolidine (383.4 mg, 5.40 mmol, 5.0 equiv.) and 4A molecular sieve at room temperature. The reaction mixture was stirred for 2 hours at 80° C. Then it was cooled to room temperature and concentrated. Into the resulting mixture was added methanol (4 mL) and sulfur (86.4 mg, 2.70 mmol, 2.5 equiv.) at 0° C. After the mixture was stirred for 10 minutes at room temperature, cyanamide (222.4 mg, 2.70 mmol, 2.5 equiv.) was added. Then the mixture was stirred overnight at room temperature. The mixture concentrated, diluted with water, and extracted with ethyl acetate (3×80 mL). The combined organic layers were washed with brine, dried over anhydrous Na₂SO₄. The resulting mixture was concentrated under vacuum. The crude product was purified by silica gel column chromatography (PE/EA=4:1) to afford the title compound (40 mg, 11%) as a yellow solid. ¹H NMR (400 MHz, Chloroform-d) δ 7.62 (d, J=7.9 Hz, 1H), 7.20 (d, J=8.1 Hz, 1H), 7.11 (s, 1H), 5.08 (s, 2H), 4.82 (d, J=8.4 Hz, 1H), 4.19-3.96 (m, 1H), 2.91 (d, J=10.5 Hz, 2H), 2.64-2.49 (m, 2H), 1.45 (s, 9H).

### Example 131C: tert-butyl(2-(4,6-dimethoxypyrimidine-5-carboxamido)-7-(trifluoromethyl)spiro[chromeno[4,3-d]thiazole-4,1′-cyclobutan]-3′-yl)carbamate

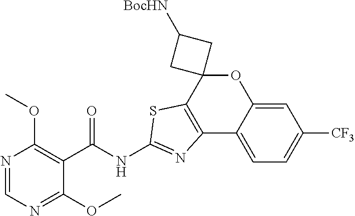 [SMILES: COc1ncnc(OC)c1C(=O)Nc1nc2c(s1)C1(CC(NC(=O)OC(C)(C)C)C1)Oc1cc(C(F)(F)F)ccc1-2]

To a stirred solution of tert-butyl(2-amino-7-(trifluoromethyl)spiro[chromeno[4,3-d]thiazole-4,1′-cyclobutan]-3′-yl)carbamate (20 mg, 0.046 mmol, 1.0 equiv.) in MeCN (1 mL) was added 4,6-dimethoxypyrimidine-5-carboxylic acid (12.4 mg, 0.069 mmol, 1.5 equiv.), TCFH (19.3 mg, 0.069 mmol, 1.5 equiv.) and NMI (13.2 mg, 0.161 mmol, 3.5 equiv.) at room temperature. The reaction mixture was stirred overnight at 50° C. After concentration, resulting residue was diluted with water, extracted with ethyl acetate (3×10 mL). The combined organic layers were washed with brine, dried over anhydrous Na₂SO₄. The resulting mixture was concentrated under vacuum. The crude product was purified by Prep-TLC (DCM/MeOH=50:1) to afford the title compound (2.2 mg, 14%) as a white solid. ESI-MS m/z=594.07 [M+H]⁺; Calculated MW: 593.15. ¹H NMR (400 MHz, Chloroform-d) δ 10.55 (s, 1H), 8.40 (s, 1H), 7.64 (d, J=7.8 Hz, 1H), 7.20 (d, J=1.6 Hz, 1H), 7.19-7.16 (m, 1H), 4.84 (s, 1H), 4.23 (d, J=14.2 Hz, 1H), 4.06 (s, 6H), 2.99 (s, 2H), 2.78-2.58 (m, 2H), 1.46 (s, 9H).

### Example 132: tert-butyl 2′-(4,6-dimethoxypyrimidine-5-carboxamido)-7′-isopropylspiro[azetidine-3,4′-chromeno[4,3-d]thiazole]-1-carboxylate

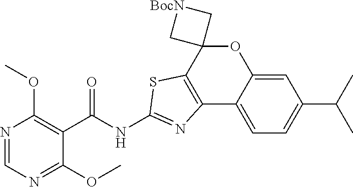 [SMILES: COc1ncnc(OC)c1C(=O)Nc1nc2c(s1)C1(CN(C(=O)OC(C)(C)C)C1)Oc1cc(C(C)C)ccc1-2]

### Example 132A: tert-butyl 7′-bromo-4′-oxospiro[azetidine-3,2′-chromane]-1-carboxylate

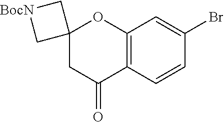 [SMILES: CC(C)(C)OC(=O)N1CC2(CC(=O)c3ccc(Br)cc3O2)C1]

To a stirred solution of 1-(4-bromo-2-hydroxyphenyl)ethan-1-one (1 g, 4.65 mmol, 1.0 equiv.) in MeOH (10 mL) was added tert-butyl 3-oxoazetidine-1-carboxylate (1.6 g, 9.30 mol, 2.0 equiv.) and pyrrolidine (660 mg, 9.30 mmol, 2.0 equiv.) at 0° C. The reaction mixture was stirred overnight at 60° C. After concentration, the resulting residue was diluted with water, extracted with ethyl acetate (3×80 mL). The combined organic layers were washed with brine, dried over anhydrous Na₂SO₄. The resulting mixture was concentrated under vacuum. The crude product was purified by silica gel column chromatography (PE:EA=10:1) to afford the title compound (950 mg, 55.7%) as a pink solid. ¹H NMR (400 MHz, Chloroform-d) δ 7.71 (d, J=8.4 Hz, 1H), 7.26 (d, J=1.9 Hz, 1H), 7.20 (dd, J=8.4, 1.8 Hz, 1H), 4.06 (d, J=9.5 Hz, 2H), 3.94 (dd, J=9.5, 1.0 Hz, 2H), 3.01 (s, 2H), 1.43 (s, 9H).

### Example 132B: tert-butyl 2′-amino-7′-bromospiro[azetidine-3,4′-chromeno[4,3-d]thiazole]-1-carboxylate

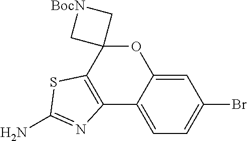 [SMILES: CC(C)(C)OC(=O)N1CC2(C1)Oc1cc(Br)ccc1-c1nc(N)sc12]

To a stirred solution of tert-butyl 7′-bromo-4′-oxospiro[azetidine-3,2′-chromane]-1-carboxylate (900 mg, 2.45 mmol, 1.0 equiv.) in PhMe (10 mL) was added p-TsOH (93.1 mg, 0.49 mmol, 0.2 equiv.) and pyrrolidine (869.7 mg, 12.25 mmol, 5.0 equiv.) at room temperature. The reaction mixture was stirred for 2 hours at 120° C. Then it was cooled to room temperature and the mixture was concentrated. Then the resulting mixture was dissolved in methanol (10 mL) and sulfur (196.2 mg, 6.13 mmol, 2.5 equiv.) was added. The mixture was stirred for 10 minutes at room temperature. Then cyanamide was added (504.8 mg, 6.13 mmol, 2.5 equiv.) and the mixture was stirred overnight at room temperature. After concentration, the resulting residue was extracted with ethyl acetate (3×80 mL). The combined organic layers were washed with brine, dried over anhydrous Na₂SO₄. The resulting mixture was concentrated under vacuum. The crude product was purified by silica gel column chromatography (PE/EA=4:1) to afford the title compound (304 mg, 45.7%) as a yellow solid. ¹H NMR (400 MHz, Chloroform-d) δ 7.45-7.38 (m, 1H), 7.12 (dq, J=3.9, 1.9 Hz, 2H), 5.14 (s, 2H), 4.35 (dd, J=9.8, 1.1 Hz, 2H), 4.20-4.02 (m, 2H), 1.46 (s, 9H).

### Example 132C: tert-butyl 7′-bromo-2′-(4,6-dimethoxypyrimidine-5-carboxamido)spiro[azetidine-3,4′-chromeno[4,3-d]thiazole]-1-carboxylate

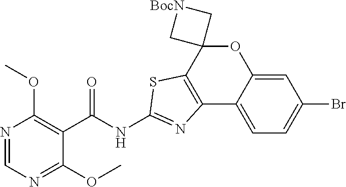 [SMILES: COc1ncnc(OC)c1C(=O)Nc1nc2c(s1)C1(CN(C(=O)OC(C)(C)C)C1)Oc1cc(Br)ccc1-2]

To a suspension of 4,6-dimethoxypyrimidine-5-carboxylic acid (192.6 mg, 1.07 mmol, 1.5 equiv.) in DCM (5 mL) and catalytic amount of DMF, was added oxalyl chloride (450.8 mg, 3.55 mmol, 5.0 equiv.) dropwise at 0° C., and the mixture was stirred for 2 hours at room temperature. The reaction mixture was concentrated in vacuo and dissolved in THF. To a stirred solution of tert-butyl 2′-amino-7′-bromospiro[azetidine-3,4′-chromeno[4,3-d]thiazole]-1-carboxylate (300 mg, 0.71 mmol, 1.0equiv.) in THF (5.0 mL) was added KOH (119.3 mg, 2.13 mmol, 3.0 equiv.) and acyl chloride/THF at 0° C., then the mixture was stirred overnight at room temperature. The resulting residue was diluted with water, extracted with ethyl acetate (3×50 mL). The combined organic layers were washed with brine, dried over anhydrous Na₂SO₄. The resulting mixture was concentrated under vacuum. The crude product was purified by Prep-TLC (DCM:EA=10:1) to afford the title compound (120 mg, 28% yield) as a white solid. ¹H NMR (400 MHz, Chloroform-d) δ 10.90 (s, 1H), 8.36 (s, 1H), 7.38 (d, J=8.2 Hz, 1H), 7.15 (d, J=1.8 Hz, 1H), 7.07 (dd, J=8.1, 1.9 Hz, 1H), 4.41 (dd, J=9.7, 1.0 Hz, 2H), 4.26-4.15 (m, 2H), 4.02 (s, 6H), 1.48 (s, 9H).

### Example 132D: tert-butyl 2′-(4,6-dimethoxypyrimidine-5-carboxamido)-7′-(prop-1-en-2-yl)spiro[azetidine-3,4′-chromeno[4,3-d]thiazole]-1-carboxylate

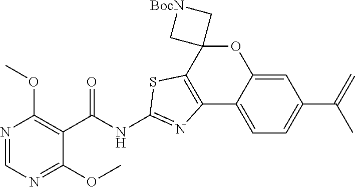 [SMILES: C=C(C)c1ccc2c(c1)OC1(CN(C(=O)OC(C)(C)C)C1)c1sc(NC(=O)c3c(OC)ncnc3OC)nc1-2]

To a stirred solution of tert-butyl 7′-bromo-2′-(4,6-dimethoxypyrimidine-5-carboxamido)spiro[azetidine-3,4′-chromeno[4,3-d]thiazole]-1-carboxylate (25 mg, 0.042 mmol, 1.0equiv.) in dioxane/H₂O (1 mL/0.2 mL) was added 4,4,5,5-tetramethyl-2-(prop-1-en-2-yl)-1,3,2-dioxaborolane (35.3 mg, 0.210 mmol, 5.0equiv.), Pd(dppf)Cl₂ (8.8 mg, 0.012 mmol, 0.3equiv.) and K₂CO₃ (11.6 mg, 0.084 mmol, 2.0equiv.) at room temperature under argon atmosphere. The reaction mixture was stirred overnight at 80° C. Then the mixture was cooled to room temperature and extracted with ethyl acetate (3×20 mL). The combined organic layers were washed with brine, dried over anhydrous Na₂SO₄. The resulting mixture was concentrated under vacuum. The crude product was purified by Prep-TLC (PE:EA=2:1) to afford the title compound (21.6 mg, 93%) as a white solid. ¹H NMR (400 MHz, Chloroform-d) δ 11.58 (s, 1H), 8.20 (s, 1H), 7.35 (d, J=7.9 Hz, 1H), 7.08 (d, J=1.7 Hz, 1H), 7.01 (dd, J=8.0, 1.7 Hz, 1H), 5.41 (s, 1H), 5.11 (t, J=1.5 Hz, 1H), 4.43 (d, J=9.7 Hz, 2H), 4.23 (d, J=9.8 Hz, 2H), 4.10 (q, J=7.0 Hz, 1H), 3.90 (s, 6H), 2.13 (d, J=1.2 Hz, 3H), 1.48 (s, 9H).

### Example 132E: tert-butyl 2′-(4,6-dimethoxypyrimidine-5-carboxamido)-7′-isopropylspiro[azetidine-3,4′-chromeno[4,3-d]thiazole]-1-carboxylate

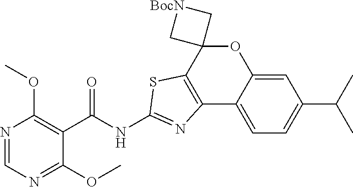 [SMILES: COc1ncnc(OC)c1C(=O)Nc1nc2c(s1)C1(CN(C(=O)OC(C)(C)C)C1)Oc1cc(C(C)C)ccc1-2]

To a stirred solution of tert-butyl 2′-(4,6-dimethoxypyrimidine-5-carboxamido)-7′-(prop-1-en-2-yl)spiro[azetidine-3,4′-chromeno[4,3-d]thiazole]-1-carboxylate (22 mg, 0.04 mmol, 1.0 equiv.) in MeOH was added Pd/C (10%, 22 mg) at room temperature under argon atmosphere. The reaction mixture was stirred overnight at room temperature. The resulting mixture was filtered and the filtrate was concentrated under reduced pressure. The crude product was purified by Prep-TLC (PE:EA=2:1) to afford the title compound (11.3 mg, 50%) as a white solid. ESI-MS m/z=554.12 [M+H]⁺; Calculated MW: 553.20. ¹H NMR (400 MHz, Chloroform-d) δ 10.90 (s, 1H), 8.33 (s, 1H), 7.42 (d, J=7.7 Hz, 1H), 6.86 (d, J=1.6 Hz, 1H), 6.81 (dd, J=7.9, 1.7 Hz, 1H), 4.45-4.38 (m, 2H), 4.22 (d, J=9.7 Hz, 2H), 4.01 (s, 6H), 3.01-2.80 (m, 1H), 1.48 (s, 9H), 1.25 (d, J=6.9 Hz, 6H).

### Example 133: ethyl 2-(4,6-dimethoxypyrimidine-5-carboxamido)-7-(trifluoromethyl)spiro[chromeno[4,3-d]thiazole-4,1′-cyclobutane]-3′-carboxylate

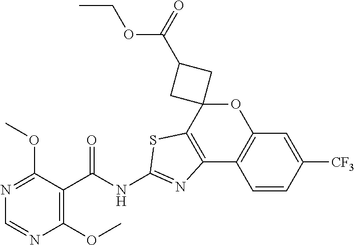 [SMILES: CCOC(=O)C1CC2(C1)Oc1cc(C(F)(F)F)ccc1-c1nc(NC(=O)c3c(OC)ncnc3OC)sc12]

### Example 133A: ethyl 4-oxo-7-(trifluoromethyl)spiro[chromane-2,1′-cyclobutane]-3′-carboxylate

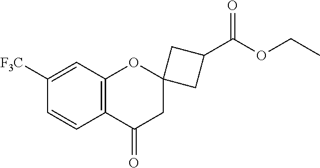 [SMILES: CCOC(=O)C1CC2(CC(=O)c3ccc(C(F)(F)F)cc3O2)C1]

To a stirred solution of 1-(2-hydroxy-4-(trifluoromethyl)phenyl)ethan-1-one (1.00 g, 4.90 mmol, 1.00 equiv.) and ethyl 3-oxocyclobutane-1-carboxylate (1.04 g, 7.35 mmol, 1.50 equiv.) in EtOH (10.00 mL) was added pyrrolidine (696.0 mg, 9.80 mmol, 2.00 equiv.) in portions at 0° C. The resulting mixture was stirred for 16 h at 60° C. under argon atmosphere. The reaction was diluted with water (300 mL), extracted with EtOAc (3×80 mL). The combined organic layers were washed with brine (2×50 mL), dried over anhydrous Na₂SO₄. After filtration, the filtrate was concentrated under reduced pressure. The residue was purified by prep-TLC (PE/EtOAc (10:1)) to afford the title compound (170 mg, 10.6% yield) as a light yellow oil. ¹H NMR (400 MHz, Chloroform-d) δ 7.94 (d, J=8.1 Hz, 1H), 7.28 (d, J=1.7 Hz, 1H), 7.23 (dd, J=8.1, 1.7 Hz, 1H), 4.15 (qd, J=7.1, 0.9 Hz, 2H), 3.35-3.24 (m, 1H), 2.98 (d, J=0.9 Hz, 2H), 2.64-2.55 (m, 2H), 2.54-2.44 (m, 2H), 1.25 (td, J=7.1, 0.9 Hz, 3H).

### Example 133B: ethyl 2-amino-7-(trifluoromethyl)spiro[chromeno[4,3-d]thiazole-4,1′-cyclobutane]-3′-carboxylate

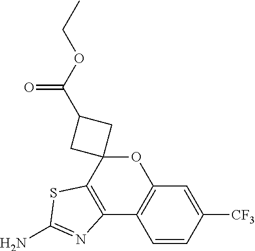 [SMILES: CCOC(=O)C1CC2(C1)Oc1cc(C(F)(F)F)ccc1-c1nc(N)sc12]

Into a 50 mL round-bottom flask were added ethyl 4-oxo-7-(trifluoromethyl)spiro[chromane-2,1′-cyclobutane]-3′-carboxylate (170 mg, 0.518 mmol, 1.00 equiv.), hexane (4.00 mL), 4A molecular sieve, pyrrolidine (184.0 mg, 2.59 mmol, 5.00 equiv.), and TsOH·H₂O (10.0 mg, 0.051 mmol, 0.10 equiv.) at room temperature. The resulting mixture was stirred for 3 h at 80° C. under argon atmosphere, cooled down to room temperature, then concentrated under reduced pressure. To this mixture was added EtOH (2.00 mL), S (42.0 mg, 1.295 mmol, 2.50 equiv.). The resulting mixture was stirred for 0.5 h at 0° C. under argon atmosphere and NH₂CN (107.00 mg, 1.295 mmol, 2.50 equiv., 51% w.t. in H₂O) was added dropwise at 0° C. The resulting mixture was stirred for additional 16 h at 0° C. to room temperature. The reaction was concentrated under reduced pressure. The residue was diluted with water, extracted with EtOAc (3×50 mL). The combined organic layers were washed with brine (2×20 mL), dried over anhydrous Na₂SO₄. After filtration, the filtrate was concentrated under reduced pressure. The residue was purified by prep-TLC (PE/EtOAc (2:1)) to afford the title compound (40.0 mg, 20% yield) as a light yellow solid. ESI-MS m/z=385.07 [M+H]⁺; Calculated MW: 384.08.

### Example 133C: ethyl 2-(4,6-dimethoxypyrimidine-5-carboxamido)-7-(trifluoromethyl)spiro[chromeno[4,3-d]thiazole-4,1′-cyclobutane]-3′-carboxylate

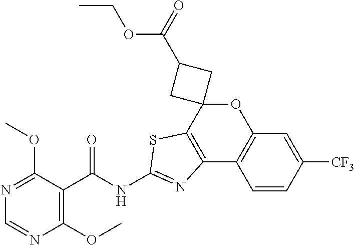 [SMILES: CCOC(=O)C1CC2(C1)Oc1cc(C(F)(F)F)ccc1-c1nc(NC(=O)c3c(OC)ncnc3OC)sc12]

To a stirred solution of 4,6-dimethoxypyrimidine-5-carboxylic acid (29.0 mg, 0.156 mmol, 1.50 equiv.) in DCM (2.0 mL) was added (COCl)₂ (66.0 mg, 0.520 mmol, 5.00 equiv.) and DMF (0.5 mg) in DCM (0.1 mL) in portions at 0° C. The resulting mixture was stirred for 3 h at room temperature under argon atmosphere, concentrated under reduced pressure and dissolved with THF (1 mL). To a stirred solution of ethyl 2-amino-7-(trifluoromethyl)spiro[chromeno[4,3-d]thiazole-4,1′-cyclobutane]-3′-carboxylate (40.0 mg, 0.104 mmol, 1.00 equiv.) in THF (1.0 mL) was added TEA (53.0 mg, 0.520 mmol, 5.00 equiv.), DMAP (13.0 mg, 0.104 mmol, 1.00 equiv.), and the acyl chloride/THF in portions at 0° C. The resulting mixture was stirred for 16 h at room temperature under argon atmosphere. The reaction was quenched with sat. NH₄Cl(aq.) at 0° C., extracted with EtOAc (3×20 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (8.0 mg, 14% yield) as a light yellow solid. ESI-MS m/z=551.12 [M+H]⁺; Calculated MW: 550.11; ¹H NMR (400 MHz, Chloroform-d) δ 11.12 (s, 1H), 8.32-8.18 (m, 1H), 7.56 (dd, J=9.2, 7.9 Hz, 1H), 7.20 (dd, J=10.0, 1.7 Hz, 1H), 7.17-7.12 (m, 1H), 4.20 (qd, J=7.1, 2.2 Hz, 2H), 3.98 (d, J=9.6 Hz, 6H), 3.45 (p, J=8.6 Hz, 1H), 3.12-2.93 (m, 2H), 2.78 (ddd, J=12.5, 8.1, 2.3 Hz, 2H), 1.30 (td, J=7.1, 3.2 Hz, 3H).

### Example 134: N—(7-bromospiro[chromeno[4,3-d]thiazole-4,3′-oxetan]-2-yl)-4,6-dimethoxypyrimidine-5-carboxamide

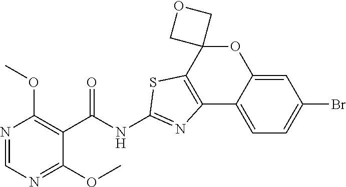 [SMILES: COc1ncnc(OC)c1C(=O)Nc1nc2c(s1)C1(COC1)Oc1cc(Br)ccc1-2]

### Example 134A: 7-bromospiro[chromane-2,3′-oxetan]-4-one

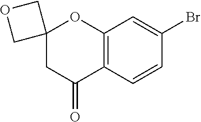 [SMILES: O=C1CC2(COC2)Oc2cc(Br)ccc21]

To a stirred solution of 1-(4-bromo-2-hydroxyphenyl)ethan-1-one (3 g, 13.9 mmol, 1.0equiv.) in MeCN (30 mL) was added oxetan-3-one (2 g, 27.8 mol, 2.0 equiv.) and pyrrolidine (2 g, 27.8 mmol, 2.0 equiv.) at 0° C. The reaction mixture was stirred overnight at 40° C. and concentrated. The resulting residue was diluted with water, extracted with ethyl acetate (3×80 mL). The combined organic layers were washed with brine, dried over anhydrous Na₂SO₄. The resulting mixture was concentrated under vacuum. The crude product was purified by silica gel column chromatography (PE:EA=10:1) to afford the title compound (1.4 g, 37.6% yield) as a yellow solid. ¹H NMR (400 MHz, Chloroform-d) δ 7.70 (d, J=8.4 Hz, 1H), 7.29 (d, J=1.7 Hz, 1H), 7.19 (dd, J=8.4, 1.7 Hz, 1H), 4.79 (d, J=7.3 Hz, 2H), 4.61-4.56 (m, 2H), 3.13 (s, 2H).

### Example 134B: 7-bromospiro[chromeno[4,3-d]thiazole-4,3′-oxetan]-2-amine

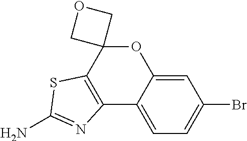 [SMILES: Nc1nc2c(s1)C1(COC1)Oc1cc(Br)ccc1-2]

To a stirred solution of 7-bromospiro[chromane-2,3′-oxetan]-4-one (1.6 g, 5.97 mmol, 1.0equiv.) in PhMe (16 mL) was added p-TsOH (114 mg, 0.60 mmol, 0.1equiv.) and pyrrolidine (2.1 g, 29.85 mmol, 5.0equiv.) at room temperature. The reaction mixture was stirred for 2 hours at 130° C. Then it was cooled to room temperature and concentrated. The resulting mixture was dissolved in MeOH (16 mL), and sulfur (477.8 mg, 14.93 mmol, 2.5 equiv.) was added. After stirring for 10 minutes at room temperature, cyanamide (1.2 g, 14.93 mmol, 2.5 equiv.) was added to the above mixture. Then the mixture was stirred overnight at room temperature. After concentration, the resulting residue was extracted with ethyl acetate (3×80 mL). The combined organic layers were washed with brine, dried over anhydrous Na₂SO₄. The resulting mixture was concentrated under vacuum. The crude product was purified by silica gel column chromatography (PE/EA=4:1) to afford the title compound (220 mg, 16.4% yield) as a yellow solid. ¹H NMR (400 MHz, DMSO-d₆) δ 7.48 (s, 2H), 7.31 (d, J=8.1 Hz, 1H), 7.18 (d, J=1.9 Hz, 1H), 7.14 (dd, J=8.1, 1.9 Hz, 1H), 4.85 (d, J=7.7 Hz, 2H), 4.76-4.68 (m, 2H).

### Example 134C: N—(7-bromospiro[chromeno[4,3-d]thiazole-4,3′-oxetan]-2-yl)-4,6-dimethoxypyrimidine-5-carboxamide

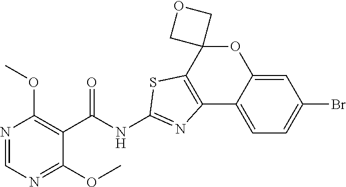 [SMILES: COc1ncnc(OC)c1C(=O)Nc1nc2c(s1)C1(COC1)Oc1cc(Br)ccc1-2]

To a suspension of 4,6-dimethoxypyrimidine-5-carboxylic acid (183.6 mg, 1.02 mmol, 1.5 equiv.) in DCM (3 mL) and catalytic amount of DMF was added oxalyl chloride (431.8 mg, 3.40 mmol, 5.0 equiv.) dropwise at 0° C. The mixture was stirred for 2 hours at room temperature. The reaction mixture was concentrated in vacuo and dissolved in THF (1 mL). To a stirred solution of 7-bromospiro[chromeno[4,3-d]thiazole-4,3′-oxetan]-2-amine (220 mg, 0.68 mmol, 1.0 equiv.) in THF (1.00 mL) was added KOH (114.2 mg, 2.04 mmol, 3.0 equiv.) in portions at 0° C. The resulting mixture was stirred for 0.5 h at 0° C. under argon atmosphere. To the above mixture was added the acyl chloride/THF dropwise at 0° C. The resulting mixture was stirred for additional 16 h at 0° C. to room temperature. The reaction was quenched with sat. NH₄Cl(aq.) at 0° C., extracted with EtOAc (3×30 mL). The combined organic layers were washed with brine, dried over anhydrous Na₂SO₄ and concentrated. The crude product was purified by Prep-TLC (PE:EA:DCM=2:1:1) to afford the title compound (70 mg, 16.3% yield) as a yellow solid. ¹H NMR (400 MHz, Chloroform-d) δ 10.67 (s, 1H), 8.44 (s, 1H), 7.47 (d, J=8.1 Hz, 1H), 7.20 (d, J=1.9 Hz, 1H), 7.12-7.10 (dd, J=8.1, 1.8 Hz, 1H), 5.10-5.07 (m, 2H), 4.91-4.87 (m, 2H), 4.10 (s, 6H).

### Example 135: N—(7-isopropylspiro[chromeno[4,3-d]thiazole-4,3′-oxetan]-2-yl)-4,6-dimethoxypyrimidine-5-carboxamide

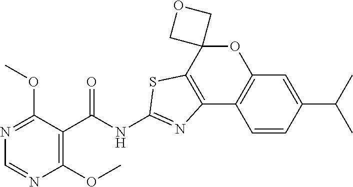 [SMILES: COc1ncnc(OC)c1C(=O)Nc1nc2c(s1)C1(COC1)Oc1cc(C(C)C)ccc1-2]

### Example 135A: 4,6-dimethoxy-N—(7-(prop-1-en-2-yl)spiro[chromeno[4,3-d]thiazole-4,3′-oxetan]-2-yl)pyrimidine-5-carboxamide

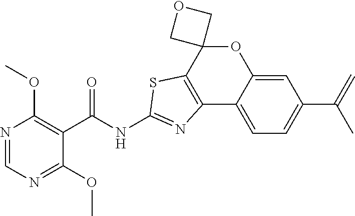 [SMILES: C=C(C)c1ccc2c(c1)OC1(COC1)c1sc(NC(=O)c3c(OC)ncnc3OC)nc1-2]

To a stirred solution of N—(7-bromospiro[chromeno[4,3-d]thiazole-4,3′-oxetan]-2-yl)-4,6-dimethoxypyrimidine-5-carboxamide (70 mg, 0.14 mmol, 1.0 equiv.) in dioxane/H₂O (2 mL/0.4 mL) was added 4,4,5,5-tetramethyl-2-(prop-1-en-2-yl)-1,3,2-dioxaborolane (117.6 mg, 0.70 mmol, 5.0equiv.), Pd(dppf)Cl₂ (29.2 mg, 0.04 mmol, 0.3equiv.) and K₂CO₃ (38.6 mg, 0.028 mmol, 2.0equiv.) at room temperature under argon atmosphere. The reaction mixture was stirred overnight at 80° C. Then the mixture was cooled down to room temperature, diluted with water, and extracted with ethyl acetate (3×10 mL). The combined organic layers were washed with brine, dried over anhydrous Na₂SO₄. The resulting mixture was concentrated under vacuum. The crude product was purified by Prep TLC (PE:EA:DCM=2:1:1) to afford the title compound (23 mg, 40% yield) as a yellow solid. ESI-MS m/z=453.05 [M+H]⁺; Calculated MW: 452.12.

### Example 135B: N—(7-isopropylspiro[chromeno[4,3-d]thiazole-4,3′-oxetan]-2-yl)-4,6-dimethoxypyrimidine-5-carboxamide

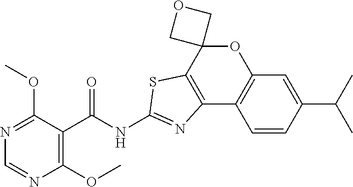 [SMILES: COc1ncnc(OC)c1C(=O)Nc1nc2c(s1)C1(COC1)Oc1cc(C(C)C)ccc1-2]

To a stirred solution of 4,6-dimethoxy-N—(7-(prop-1-en-2-yl)spiro[chromeno[4,3-d]thiazole-4,3′-oxetan]-2-yl)pyrimidine-5-carboxamide (23 mg, 0.05 mmol, 1.0equiv.) in MeOH (1 mL) was added Pd/C (10%, 46 mg) at room temperature under H₂ atmosphere. The reaction mixture was stirred overnight at room temperature. The resulting mixture was filtered, the filter cake was washed with MeOH (3×10 mL). The filtrate was concentrated under reduced pressure. The crude product was purified by Prep-HPLC to afford the title compound (6.3 mg, 31% yield) as a white solid. ESI-MS m/z=455.09 [M+H]⁺; Calculated MW: 454.13. ¹H NMR (400 MHz, Chloroform-d) δ 10.82 (s, 1H), 8.38 (s, 1H), 7.47 (d, J=7.8 Hz, 1H), 6.91 (d, J=1.6 Hz, 1H), 6.83 (dd, J=7.9, 1.7 Hz, 1H), 5.13-5.08 (m, 2H), 4.91-4.87 (m, 2H), 4.05 (s, 6H), 2.87 (p, J=6.9 Hz, 1H), 1.25 (d, J=6.9 Hz, 6H).

### Example 136: N—(1-isopropyl-7′-(trifluoromethyl)spiro[azetidine-3,4′-chromeno[4,3-d]thiazol]-2′-yl)-4,6-dimethoxypyrimidine-5-carboxamide

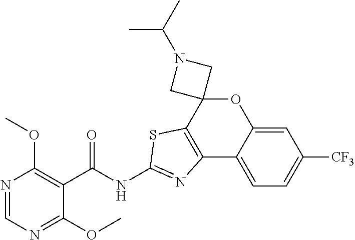 [SMILES: COc1ncnc(OC)c1C(=O)Nc1nc2c(s1)C1(CN(C(C)C)C1)Oc1cc(C(F)(F)F)ccc1-2]

To a stirred solution of 4,6-dimethoxy-N—(7′-(trifluoromethyl)spiro[azetidine-3,4′-chromeno[4,3-d]thiazol]-2′-yl)pyrimidine-5-carboxamide (20 mg, 0.04 mmol, 1.0equiv.) in DCE (1 mL) was added acetone (11.6 mg, 0.20 mol, 5.0equiv.) and AcOH (0.6 mg, 0.01 mmol, 0.1equiv.) at room temperature under argon atmosphere. The reaction mixture was stirred for 15 min at room temperature. Then NaBH(OAc)₃ (25.4 mg, 0.12 mmol, 3.0equiv.) was added at 0° C. The reaction mixture was stirred for 2 hours at room temperature. The mixture was quenched with water and extracted with DCM (3×5 mL). The combined organic layers were washed with brine, dried over anhydrous Na₂SO₄. The resulting mixture was concentrated under vacuum. The crude product was purified by prep-TLC (PE:EA=2:1) to afford the title compound (15.0 mg, 72%) as a white solid. ESI-MS m/z=522.16 [M+H]⁺; Calculated MW: 521.13. ¹H NMR (400 MHz, DMSO-d₆) δ 12.89 (s, 1H), 8.59 (s, 1H), 7.69 (d, J=7.9 Hz, 1H), 7.34 (dd, J=8.0, 1.7 Hz, 1H), 7.25 (d, J=1.7 Hz, 1H), 3.93 (s, 6H), 3.62 (d, J=8.0 Hz, 2H), 3.39-3.34 (m, 2H), 2.39 (p, J=6.1 Hz, 1H), 0.91 (d, J=6.1 Hz, 6H).

### Example 137: N—(7-cyanospiro[chromeno[4,3-d]thiazole-4,3′-oxetan]-2-yl)-4,6-dimethoxypyrimidine-5-carboxamide

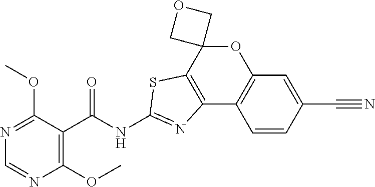 [SMILES: COc1ncnc(OC)c1C(=O)Nc1nc2c(s1)C1(COC1)Oc1cc(C#N)ccc1-2]

To a stirred solution of N—(7-bromospiro[chromeno[4,3-d]thiazole-4,3′-oxetan]-2-yl)-4,6-dimethoxypyrimidine-5-carboxamide (13 mg, 0.0268 mmol, 1.00 equiv.), Zn(CN)₂ (6.2 mg, 0.0530 mmol, 2.00 equiv.) and t-Buxphos Pd G3 (4.2 mg, 0.0053 mmol, 0.20 equiv.) in THF (0.5 mL) and H₂O (0.5 mL) at 0° C., and the mixture was stirred for overnight at 50° C. The resulting mixture was extracted with EA (2×5 mL). The combined organic layers were washed with brine (3×5 mL), dried over anhydrous Na₂SO₄. After filtration, the filtrate was concentrated under reduced pressure. The crude product was purified by silica gel chromatography (DCM:PE:EA=1:2:1) to afford the title compound (7.5 mg, 64%) as a white solid. ESI-MS m/z=438.05 [M+H]⁺; Calculated MW: 437.08. ¹H NMR (400 MHz, Chloroform-d) δ 10.24 (s, 1H), 8.51 (s, 1H), 7.73 (d, J=7.8 Hz, 1H), 7.32-7.26 (m, 2H), 5.12-5.08 (m, 2H), 4.93-4.88 (m, 2H), 4.16 (s, 6H).

### Example 138: N—(7-cyclopropylspiro[chromeno[4,3-d]thiazole-4,3′-oxetan]-2-yl)-4,6-dimethoxypyrimidine-5-carboxamide

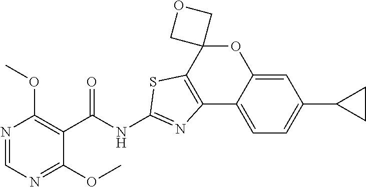 [SMILES: COc1ncnc(OC)c1C(=O)Nc1nc2c(s1)C1(COC1)Oc1cc(C3CC3)ccc1-2]

### Example 138A: 7-cyclopropylspiro[chromane-2,3′-oxetan]-4-one

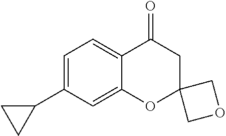 [SMILES: O=C1CC2(COC2)Oc2cc(C3CC3)ccc21]

To a stirred solution of 7-bromospiro[chromane-2,3′-oxetan]-4-one (200 mg, 0.746 mmol, 1.00 equiv.) in toluene (2 mL) and H₂O (2 mL) was added cyclopropylboronic acid (128 mg, 1.493 mmol, 2.0 equiv.), K₃PO₄ (554 mg, 2.612 mmol, 3.5 equiv.), PCy₃ (63 mg, 0.224 mmol, 0.3 equiv.) and Pd(OAc)₂ (25 mg, 0.112 mmol, 0.15 equiv.) at room temperature under argon atmosphere. The resulting mixture was stirred overnight at 100° C. The resulting mixture was extracted with EA (2×20 mL). The combined organic layers were washed with brine (1×10 mL), dried over anhydrous Na₂SO₄. After filtration, the filtrate was concentrated under reduced pressure. The crude product was purified by silica gel column chromatography (PE:EA=10:1) to afford the title compound (160 mg, 94%) as yellow oil. ¹H NMR (400 MHz, Chloroform-d) δ 7.78-7.65 (m, 1H), 6.81-6.63 (m, 2H), 4.78 (dd, J=7.3, 3.8 Hz, 2H), 4.58 (dd, J=7.3, 3.7 Hz, 2H), 3.09 (d, J=3.9 Hz, 2H), 1.97-1.78 (m, 1H), 1.13-0.99 (m, 2H), 0.79 (dq, J=7.1, 4.6 Hz, 2H).

### Example 138B: 7-cyclopropylspiro[chromeno[4,3-d]thiazole-4,3′-oxetan]-2-amine

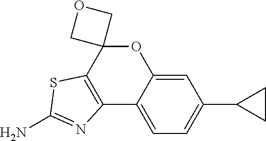 [SMILES: Nc1nc2c(s1)C1(COC1)Oc1cc(C3CC3)ccc1-2]

To a suspension of 7-cyclopropylspiro[chromane-2,3′-oxetan]-4-one (200 mg, 0.870 mmol, 1.00 equiv.) and TsOH-H₂O (17 mg, 0.087 mmol, 0.10 equiv.) in n-hexane (5 mL), pyrrolidine (309 mg, 4.348 mmol, 5.00 equiv.) was added at 0° C., and the mixture was stirred for 2 h at 70° C. Then the reaction mixture was concentrated in vacuo and dissolved in MeOH (3 mL). S (70 mg, 2.174 mmol, 2.5 equiv.) was added at 0° C. and the mixture was stirred for 10 mins at 0° C. Then 51% w/w % cyanamide (179 mg, 2.174 mmol, 2.5 equiv.) was added at 0° C. and the mixture was stirred overnight at room temperature. The resulting mixture was concentrated under vacuum and was extracted with EA (2×20 mL). The combined organic layers were washed with brine (2×10 mL), dried over anhydrous Na₂SO₄. After filtration, the filtrate was concentrated under reduced pressure. The crude product was purified by silica gel column chromatography (PE:EA=3:1) to afford the title compound (60 mg, 24%) as a yellow solid. ESI-MS m/z=287.02 [M+H]⁺; Calculated MW: 286.08.

### Example 138C: N—(7-cyclopropylspiro[chromeno[4,3-d]thiazole-4,3′-oxetan]-2-yl)-4,6-dimethoxypyrimidine-5-carboxamide

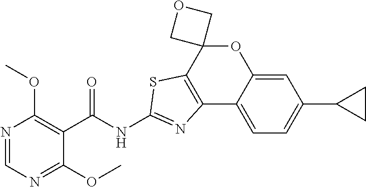 [SMILES: COc1ncnc(OC)c1C(=O)Nc1nc2c(s1)C1(COC1)Oc1cc(C3CC3)ccc1-2]

To a stirred solution of 7-cyclopropylspiro[chromeno[4,3-d]thiazole-4,3′-oxetan]-2-amine (40 mg, 0.140 mmol, 1.00 equiv.), 4,6-dimethoxypyrimidine-5-carboxylic acid (38 mg, 0.210 mmol, 1.50 equiv.) and NMI (40 mg, 0.489 mmol, 3.50 equiv.) in ACN (1 mL) was added TCFH (79 mg, 0.280 mmol, 2.00 equiv.) in ACN (0.5 mL) at 0° C., and the mixture was stirred overnight at 60° C. The mixture was quenched with NaHCO₃(aq), then the resulting mixture was extracted with EA (2×10 mL) and washed with brine. The combined organic layers were washed with brine (1×10 mL), dried over anhydrous Na₂SO₄. After filtration, the filtrate was concentrated under reduced pressure. The crude product was purified by HPLC to afford the title compound (1.6 mg, 2.5%) as a white solid. ESI-MS m/z=453.11 [M+H]⁺; Calculated MW: 452.12. ¹H NMR (400 MHz, Chloroform-d) δ 10.30 (s, 1H), 8.53-8.41 (m, 1H), 7.49 (dt, J=7.9, 1.6 Hz, 1H), 6.73 (dp, J=9.5, 1.7 Hz, 2H), 5.07 (d, J=7.7 Hz, 2H), 4.97-4.83 (m, 2H), 4.11 (q, J=3.4, 2.4 Hz, 6H), 1.86 (d, J=7.7 Hz, 1H), 0.98 (dq, J=6.6, 1.8 Hz, 2H), 0.72 (dt, J=6.8, 1.8 Hz, 2H).

### Example 139: N—(7-(2-hydroxypropan-2-yl)spiro[chromeno[4,3-d]thiazole-4,3′-oxetan]-2-yl)-4,6-dimethoxypyrimidine-5-carboxamide

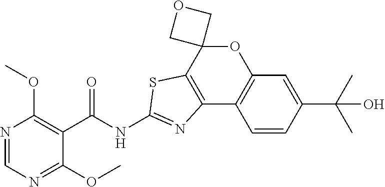 [SMILES: COc1ncnc(OC)c1C(=O)Nc1nc2c(s1)C1(COC1)Oc1cc(C(C)(C)O)ccc1-2]

To a flame-dried Schlenk tube equipped with a magnetic stir bar was charged with 4,6-dimethoxy-N—(7-(prop-1-en-2-yl)spiro[chromeno[4,3-d]thiazole-4,3′-oxetan]-2-yl)pyrimidine-5-carboxamide (200 mg, 442.0 μmol, 1.0 equiv.), methyl 4-nitrobenzenesulfonate (124.8 mg, 574.60 μmol, 1.30 equiv.), NaHCO₃ (74.3 mg, 884.01 μmol, 2.0 equiv.) and Fe(acac)₃ (15.6 mg, 44.20 μmol, 0.1 equiv.). The reaction vessel was evacuated and backfilled with argon and then dry MeOH (0.4 mL) was added under argon atmosphere. The resultant mixture was cooled to 0° C. followed by addition of PhSiH₃ (143.5 mg, 1.33 mmol, 3.0 equiv.). The mixture was stirred at rt for 16 hours. The mixture was concentrated under reduce pressure then quenched with ice-water (5 mL). The aqueous was extracted with EA (10 mL×2). The combine organic layers were washed with brine (20 mL×2), dried over Na₂SO₄, filtrated, and concentrated under reduce pressure. The crude product was purified by prep-TLC (PE:EA=1:1) to afford the title compound (90 mg, 43%) as yellow solid. ESI-MS m/z=471.17 [M+H]⁺; Calculated MW: 470.50. ¹H NMR (400 MHz, Chloroform-d) δ 11.68 (s, 1H), 8.18 (s, 1H), 7.31 (d, J=7.9 Hz, 1H), 7.04-6.87 (m, 2H), 3.92 (s, 6H), 1.94 (ddt, J=35.6, 14.3, 7.2 Hz, 4H), 1.00 (t, J=7.3 Hz, 6H).

### Example 140: N—(7-(2-cyanopropan-2-yl)spiro[chromeno[4,3-d]thiazole-4,3′-oxetan]-2-yl)-4,6-dimethoxypyrimidine-5-carboxamide

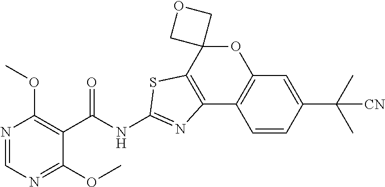 [SMILES: COc1ncnc(OC)c1C(=O)Nc1nc2c(s1)C1(COC1)Oc1cc(C(C)(C)C#N)ccc1-2]

To a 10-mL Schlenk tube were added InBr3 (2.3 mg, 63.76 μmol, 0.1 equiv.), CH₂Cl₂ (0.5 mL) and TMSCN (12.7 mg, 15.95 μL, 127.52 μmol, 2equiv.) under argon atmosphere. N—(7-(2-hydroxypropan-2-yl)spiro[chromeno[4,3-d]thiazole-4,3′-oxetan]-2-yl)-4,6-dimethoxypyrimidine-5-carboxamide (30 mg, 63.76 μmol, 1 equiv.) in CH₂Cl₂ (0.5 mL) was then introduced to the reaction system dropwise with a syringe at 0° C. The mixture was stirred at room temperature for 2 hours. The resulting yellow solution was evaporated under vacuum and the residue was purified by prep-TLC (PE:EA=1:1) to afford the title compound (3 mg, 9.8%) as white-off solid. ESI-MS m/z=480.15 [M+H]⁺; Calculated MW: 479.51. ¹H NMR (400 MHz, Chloroform-d) δ 10.63 (s, 1H), 8.40 (s, 1H), 7.77-7.51 (m, 1H), 7.21-7.07 (m, 2H), 5.10 (d, J=7.6 Hz, 2H), 4.99-4.82 (m, 2H), 4.08 (s, 6H), 1.73 (s, 6H).

### Example 141: N—(1′,1′-dioxido-7-(trifluoromethyl)-2′,3′,5′,6′-tetrahydrospiro[chromeno[3,4-d]thiazole-4,4′-thiopyran]-2-yl)-4,6-dimethoxypyrimidine-5-carboxamide

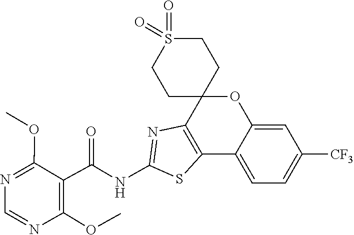 [SMILES: COc1ncnc(OC)c1C(=O)Nc1nc2c(s1)-c1ccc(C(F)(F)F)cc1OC21CCS(=O)(=O)CC1]

To a stirred solution of 4,6-dimethoxy-N—(7-(trifluoromethyl)-2′,3′,5′,6′-tetrahydrospiro[chromeno[4,3-d]thiazole-4,4′-thiopyran]-2-yl)pyrimidine-5-carboxamide (15.0 mg, 0.029 mmol, 1.00 equiv.) in DCM (1.00 mL) was added m-CPBA (10.0 mg, 0.057 mmol, 2.00 equiv.) in portions at 0° C. The resulting mixture was stirred for 16 h at room temperature under argon atmosphere. The reaction was diluted with DCM (10.00 mL), washed with NaHCO₃ (2×10 mL), brine (2×10 mL), and dried over anhydrous Na₂SO₄. After filtration, the filtrate was concentrated under reduced pressure. The residue was purified by prep-TLC (PE/EtOAc (1:1)) to afford N—(1′,1′-dioxido-7-(trifluoromethyl)-2′,3′,5′,6′-tetrahydrospiro[chromeno[4,3-d]thiazole-4,4′-thiopyran]-2-yl)-4,6-dimethoxypyrimidine-5-carboxamide (2.0 mg, 12% yield) as a white solid. ESI-MS m/z=557.07 [M+H]⁺; Calculated MW: 556.07; ¹H NMR (400 MHz, Chloroform-d) δ 10.29 (s, 1H), 8.49 (s, 1H), 7.79 (d, J=7.9 Hz, 1H), 7.35-7.29 (m, 1H), 7.26 (d, J=1.7 Hz, 1H), 4.13 (s, 6H), 3.59 (td, J=13.6, 4.2 Hz, 2H), 3.02 (d, J=13.9 Hz, 2H), 2.63 (d, J=15.8 Hz, 4H).

### Example 142: N—(4,4-diethyl-7-(trifluoromethyl)-4H-chromeno[4,3-d]thiazol-2-yl)-2,6-dimethoxybenzamide

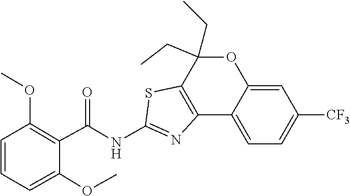 [SMILES: CCC1(CC)Oc2cc(C(F)(F)F)ccc2-c2nc(NC(=O)c3c(OC)cccc3OC)sc21]

To a stirred solution of 4,4-diethyl-7-(trifluoromethyl)-4H-chromeno[4,3-d]thiazol-2-amine (60 mg, 0.183 mmol, 1.00 equiv.), 2,6-dimethoxybenzoic acid (50 mg, 0.274 mmol, 1.50 equiv.) and NMI (53 mg, 0.640 mmol, 3.50 equiv.) in ACN (1 mL) was added TCFH (103 mg, 0.366 mmol, 2.00 equiv.) in ACN (0.5 mL) at 0° C., and the mixture was stirred overnight at 60° C. The mixture was quenched with NaHCO₃(aq), then the resulting mixture was extracted with EA (2×15 mL). The combined organic layers were washed with brine (1×10 mL), dried over anhydrous Na₂SO₄. After filtration, the filtrate was concentrated under reduced pressure. The crude product was purified by prep-TLC (PE:EA:DCM=5:1:1) to afford the title compound (16 mg, 18%) as a yellow solid. ESI-MS m/z=493.19 [M+H]⁺; Calculated MW: 492.13. ¹H NMR (400 MHz, Chloroform-d) δ 11.46 (s, 1H), 7.40 (d, J=7.8 Hz, 1H), 7.09-6.95 (m, 3H), 6.24 (d, J=8.5 Hz, 2H), 3.68 (s, 6H), 1.95 (ddq, J=44.2, 14.7, 7.4 Hz, 4H), 1.01 (t, J=7.4 Hz, 6H).

### Example 143:3-(4,4-diethyl-7-(trifluoromethyl)-4H-chromeno[4,3-d]thiazol-2-yl)-5-methoxy-1-methyl-2,3-dihydropyrimido[4,5-d]pyrimidin-4 (1H)-one

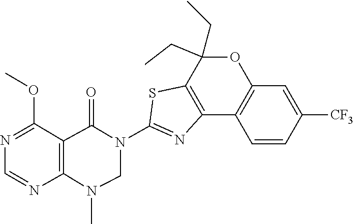 [SMILES: CCC1(CC)Oc2cc(C(F)(F)F)ccc2-c2nc(N3CN(C)c4ncnc(OC)c4C3=O)sc21]

To a stirred solution of N—(4,4-diethyl-7-(trifluoromethyl)-4H-chromeno[4,3-d]thiazol-2-yl)-4-methoxy-6-(methylamino)pyrimidine-5-carboxamide (20.0 mg, 0.041 mmol, 1.00 equiv.) and Cs₂CO₃ (53.00 mg, 0.162 mmol, 4.00 equiv.) in ACN (1.00 mL) was added CH₂₁₂ (33.00 mg, 0.122 mmol, 3.00 equiv.) in portions at room temperature. The resulting mixture was stirred for 2 h at 90° C. under argon atmosphere. The reaction was diluted with water (15 mL), extracted with EtOAc (3×5 mL). The combined organic layers were washed with brine (2×5 mL) and dried over anhydrous Na₂SO₄. After filtration, the filtrate was concentrated under reduced pressure. The residue was purified by prep-TLC (PE/EtOAc (4:1)) to afford the title compound (2.0 mg, 9.7% yield) as a white solid. ESI-MS m/z=506.20 [M+H]⁺; Calculated MW: 505.14; ¹H NMR (400 MHz, Chloroform-d) δ 8.60 (s, 1H), 7.66 (dd, J=8.0, 1.1 Hz, 1H), 7.14 (ddd, J=8.0, 1.8, 0.8 Hz, 1H), 7.04 (d, J=1.8 Hz, 1H), 6.44 (d, J=13.5 Hz, 1H), 4.16 (s, 3H), 3.89 (d, J=14.1 Hz, 1H), 3.84 (s, 3H), 2.05 (m, 2H), 1.79 (d, J=30.5 Hz, 2H), 0.94 (t, J=7.4 Hz, 6H).

### Example 144:4-hydroxy-N—(7-isopropylspiro[chromeno[4,3-d]thiazole-4,3′-oxetan]-2-yl)-6-methoxypyrimidine-5-carboxamide

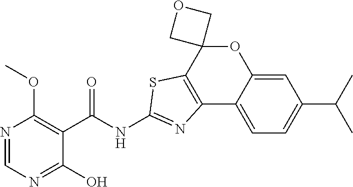 [SMILES: COc1ncnc(O)c1C(=O)Nc1nc2c(s1)C1(COC1)Oc1cc(C(C)C)ccc1-2]

To a stirred solution of 7-isopropylspiro[chromeno[4,3-d]thiazole-4,3′-oxetan]-2-amine (18 mg, 0.063 mmol, 1.00 equiv.), 4-hydroxy-6-methoxypyrimidine-5-carboxylic acid (16 mg, 0.094 mmol, 1.50 equiv.) and NMI (18 mg, 0.219 mmol, 3.50 equiv.) in ACN (1 mL) was added TCFH (35.1 mg, 0.125 mmol, 2.00 equiv.) in ACN (1 mL) at 0° C., and the mixture was stirred overnight at 60° C. The mixture was quenched with NaHCO₃(aq), then the resulting mixture was extracted with EA (2×20 mL). The combined organic layers were washed with brine (1×10 mL), dried over anhydrous Na₂SO₄. After filtration, the filtrate was concentrated under reduced pressure. The crude product was purified by prep-TLC (PE:EA:DCM=4:1:1) to afford the title compound (7 mg, 23%) as a yellow solid. ESI-MS m/z=441.12 [M+H]⁺; Calculated MW: 440.12. ¹H NMR (400 MHz, DMSO-d₆) δ 13.33 (s, 1H), 12.97 (s, 1H), 8.40 (s, 1H), 7.49 (d, J=7.6 Hz, 1H), 6.94-6.86 (m, 2H), 4.92 (d, J=7.5 Hz, 2H), 4.80 (d, J=7.5 Hz, 2H), 3.96 (s, 3H), 2.83 (p, J=6.8 Hz, 1H), 1.16 (d, J=6.8 Hz, 6H).

### Example 145: N—(1,7′-diisopropylspiro[azetidine-3,4′-chromeno[4,3-d]thiazol]-2′-yl)-4,6-dimethoxypyrimidine-5-carboxamide

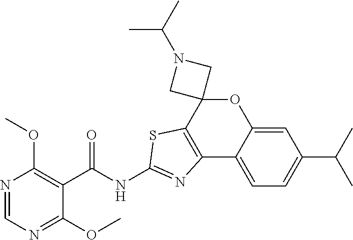 [SMILES: COc1ncnc(OC)c1C(=O)Nc1nc2c(s1)C1(CN(C(C)C)C1)Oc1cc(C(C)C)ccc1-2]

### Example 145A: tert-butyl 7′-isopropyl-4′-oxospiro[azetidine-3,2′-chromane]-1-carboxylate)

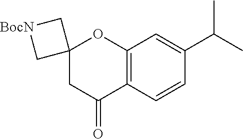 [SMILES: CC(C)c1ccc2c(c1)OC1(CC2=O)CN(C(=O)OC(C)(C)C)C1]

To a stirred solution of 1-(2-hydroxy-4-isopropylphenyl)ethan-1-one (1 g, 5.62 mmol, 1.0 equiv.) in MeOH (10 mL) was added tert-butyl 3-oxoazetidine-1-carboxylate (1.4 g, 8.43 mmol, 1.5 equiv.) and pyrrolidine (0.8 g, 11.24 mmol, 2.0 equiv.) at 0° C. The reaction mixture was stirred overnight at 60° C. under argon atmosphere and concentrated. The resulting residue was diluted with water, extracted with ethyl acetate (3×100 mL). The combined organic layers were washed with brine, dried over anhydrous Na₂SO₄. The resulting mixture was concentrated under vacuum. The crude product was purified by silica gel column chromatography (PE:EA=20:1) to afford the title compound (540 mg, 29.0%) as a brown oil. ¹H NMR (400 MHz, Chloroform-d) δ 7.77 (dd, J=8.1, 1.0 Hz, 1H), 6.92 (dt, J=8.1, 1.3 Hz, 1H), 6.88 (d, J=1.5 Hz, 1H), 4.06 (d, J=9.5 Hz, 2H), 3.97-3.90 (m, 2H), 2.98 (s, 2H), 2.88 (tt, J=11.1, 6.9 Hz, 1H), 1.43 (d, J=1.0 Hz, 9H), 1.25-1.23 (m, 6H).

### Example 145B: tert-butyl 2′-amino-7′-isopropylspiro[azetidine-3,4′-chromeno[4,3-d]thiazole]-1-carboxylate

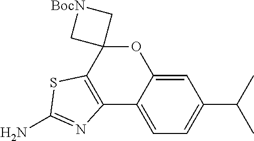 [SMILES: CC(C)c1ccc2c(c1)OC1(CN(C(=O)OC(C)(C)C)C1)c1sc(N)nc1-2]

To a stirred solution of tert-butyl 7′-isopropyl-4′-oxospiro[azetidine-3,2′-chromane]-1-carboxylate (500 mg, 1.51 mmol, 1.0 equiv.) in PhMe (10 mL) was added p-TsOH (28.5 mg, 0.15 mmol, 0.1 equiv.) and pyrrolidine (536.1 mg, 7.55 mmol, 5.0 equiv.) at room temperature. The reaction mixture was stirred for 2 hours at 130° C. Then it was cooled to room temperature, concentrated, and dissolved in MeOH (5 mL). After the addition of sulfur (120.9 mg, 3.78 mmol, 2.5 equiv.), the mixture was stirred for 30 minutes at room temperature. Then cyanamide (311.0 mg, 3.78 mmol, 2.5 equiv.) was added. Then the mixture was stirred overnight at room temperature. After concentration, the resulting residue was diluted with water, extracted with ethyl acetate (3×80 mL). The combined organic layers were washed with brine, dried over anhydrous Na₂SO₄. The resulting mixture was concentrated under vacuum. The crude product was purified by silica gel column chromatography (PE/EA=4:1) to afford the title compound (80 mg, 13.7%) as a white solid. ¹H NMR (400 MHz, Chloroform-d) δ 7.47 (d, J=7.8 Hz, 1H), 6.94-6.81 (m, 2H), 5.56 (s, 2H), 4.36 (dd, J=9.7, 1.1 Hz, 2H), 4.13 (dd, J=9.7, 1.2 Hz, 2H), 2.85 (hept, J=7.3, 6.8 Hz, 1H), 1.46 (s, 9H), 1.22 (d, J=6.9 Hz, 6H).

### Example 145C: tert-butyl 2′-(4,6-dimethoxypyrimidine-5-carboxamido)-7′-isopropylspiro[azetidine-3,4′-chromeno[4,3-d]thiazole]-1-carboxylate

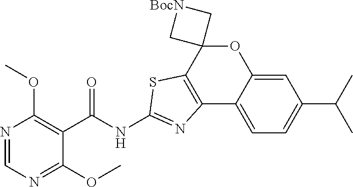 [SMILES: COc1ncnc(OC)c1C(=O)Nc1nc2c(s1)C1(CN(C(=O)OC(C)(C)C)C1)Oc1cc(C(C)C)ccc1-2]

To a stirred solution of tert-butyl 2′-amino-7′-isopropylspiro[azetidine-3,4′-chromeno[4,3-d]thiazole]-1-carboxylate (40 mg, 0.12 mmol, 1.0 equiv.) in MeCN (1 mL) was added 4,6-dimethoxypyrimidine-5-carboxylic acid (32.4 mg, 0.18 mmol, 1.5 equiv.), TCFH (50.4 mg, 0.18 mmol, 1.5equiv.) and NMI (34.4 mg, 0.42 mmol, 3.5 equiv.) at room temperature. The reaction mixture was stirred overnight at 50° C. After concentration, the resulting residue was diluted with water and extracted with ethyl acetate (3×10 mL). The combined organic layers were washed with brine, dried over anhydrous Na₂SO₄. The resulting mixture was concentrated under vacuum. The crude product was purified by Prep-TLC (PE/EA=2:1) to afford the title compound (33 mg, 49.7%) as a white solid. ¹H NMR (400 MHz, Chloroform-d) δ 10.70 (s, 1H), 8.42 (s, 1H), 7.56-7.49 (m, 1H), 6.87 (d, J=1.3 Hz, 2H), 4.41 (dd, J=9.7, 1.0 Hz, 2H), 4.25-4.18 (m, 2H), 4.09 (s, 6H), 2.86 (h, J=6.8 Hz, 1H), 1.47 (s, 9H), 1.24 (d, J=6.9 Hz, 6H).

### Example 145D: N—(7′-isopropylspiro[azetidine-3,4′-chromeno[4,3-d]thiazol]-2′-yl)-4,6-dimethoxypyrimidine-5-carboxamide

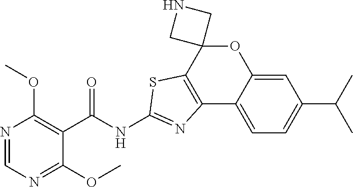 [SMILES: COc1ncnc(OC)c1C(=O)Nc1nc2c(s1)C1(CNC1)Oc1cc(C(C)C)ccc1-2]

To a stirred solution of tert-butyl 2′-(4,6-dimethoxypyrimidine-5-carboxamido)-7′-isopropylspiro[azetidine-3,4′-chromeno[4,3-d]thiazole]-1-carboxylate (30 mg, 0.14 mmol, 1.0equiv.) in DCM (1 mL) was added TFA (0.5 mL) dropwise at 0° C. under argon atmosphere. The reaction mixture was stirred for 2 hours at room temperature and concentrated. The pH of the mixture was adjusted to 9 by saturated NaHCO₃ solution at 0° C. The resulting solution was extracted with ethyl acetate (3×10 mL). The combined organic layers were washed with brine, dried over anhydrous Na₂SO₄. The resulting mixture was concentrated under vacuum to afford the title compound (25 mg, crude) as a white solid. ESI-MS m/z=454.18 [M+H]⁺; Calculated MW: 453.15.

### Example 145E: N—(1,7′-diisopropylspiro[azetidine-3,4′-chromeno[4,3-d]thiazol]-2′-yl)-4,6-dimethoxypyrimidine-5-carboxamide

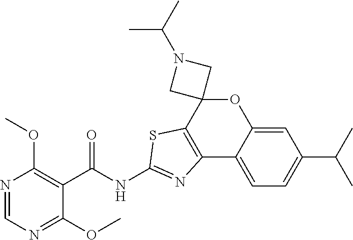 [SMILES: COc1ncnc(OC)c1C(=O)Nc1nc2c(s1)C1(CN(C(C)C)C1)Oc1cc(C(C)C)ccc1-2]

To a stirred solution of N—(7′-isopropylspiro[azetidine-3,4′-chromeno[4,3-d]thiazol]-2′-yl)-4,6-dimethoxypyrimidine-5-carboxamide (24 mg, 0.05 mmol, 1.0equiv.) in DCE (1 mL) was added acetone (14.5 mg, 0.25 mol, 5.0equiv.) and AcOH (0.6 mg, 0.01 mmol, 0.1equiv.) at room temperature under argon atmosphere. The reaction mixture was stirred for 15 min at room temperature. To the above solution was added NaBH(OAc)₃ (31.8 mg, 0.15 mmol, 3.0equiv.) at 0° C. The reaction mixture was stirred for 2 hours at room temperature. The mixture was diluted with water, extracted with DCM (3×5 mL). The combined organic layers were washed with brine, dried over anhydrous Na₂SO₄. The resulting mixture was concentrated under vacuum. The crude product was purified by prep-TLC (DCM:EA=5:1) to afford the title compound (11.0 mg, 44%) as a white solid. ESI-MS m/z=496.30 [M+H]⁺; Calculated MW: 495.19. ¹H NMR (400 MHz, DMSO-d₆) δ 12.79 (s, 1H), 8.58 (s, 1H), 7.43 (d, J=7.8 Hz, 1H), 6.91-6.84 (m, 1H), 6.82 (d, J=1.6 Hz, 1H), 3.92 (s, 6H), 3.55 (d, J=7.1 Hz, 2H), 3.32 (s, 2H), 2.80 (p, J=6.8 Hz, 1H), 2.37 (t, J=6.1 Hz, 1H), 1.15 (d, J=6.9 Hz, 6H), 0.90 (d, J=6.1 Hz, 6H).

### Example 146: N—(7′-bromo-1-isopropylspiro[azetidine-3,4′-chromeno[4,3-d]thiazol]-2′-yl)-4,6-dimethoxypyrimidine-5-carboxamide

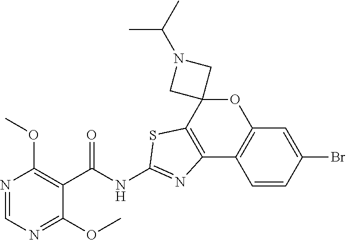 [SMILES: COc1ncnc(OC)c1C(=O)Nc1nc2c(s1)C1(CN(C(C)C)C1)Oc1cc(Br)ccc1-2]

### Example 146A: N—(7′-bromospiro[azetidine-3,4′-chromeno[4,3-d]thiazol]-2′-yl)-4,6-dimethoxypyrimidine-5-carboxamide

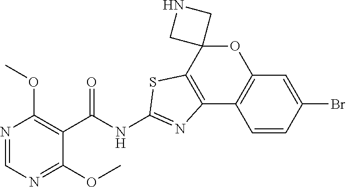 [SMILES: COc1ncnc(OC)c1C(=O)Nc1nc2c(s1)C1(CNC1)Oc1cc(Br)ccc1-2]

To a stirred solution of tert-butyl 2′-(4,6-dimethoxypyrimidine-5-carboxamido)-7′-isopropylspiro[azetidine-3,4′-chromeno[4,3-d]thiazole]-1-carboxylate (85. mg, 0.144 mmol, 1.00 equiv.) in DCM (1.50 mL) was added TFA (0.5 mL) in portions at 0° C. The resulting mixture was stirred for 2 h at room temperature. The reaction was diluted with toluene (2.0 mL), then concentrated under reduced pressure to provide the title compound (96.0 mg, TFA salt, crude) as a yellow solid. ¹H NMR (400 MHz, Chloroform-d) δ 8.21 (s, 1H), 7.22 (d, J=6.8 Hz, 1H), 7.11 (dd, J=4.1, 1.9 Hz, 1H), 6.96 (dd, J=8.1, 1.9 Hz, 1H), 4.19-4.07 (m, 2H), 3.90 (s, 6H), 3.87 (d, J=2.6 Hz, 2H).

### Example 146B: N—(7′-bromo-1-isopropylspiro[azetidine-3,4′-chromeno[4,3-d]thiazol]-2′-yl)-4,6-dimethoxypyrimidine-5-carboxamide

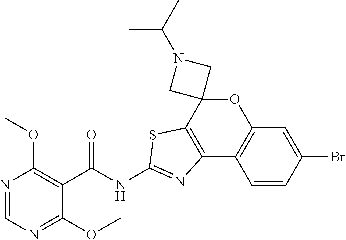 [SMILES: COc1ncnc(OC)c1C(=O)Nc1nc2c(s1)C1(CN(C(C)C)C1)Oc1cc(Br)ccc1-2]

To a stirred solution of N—(7′-bromospiro[azetidine-3,4′-chromeno[4,3-d]thiazol]-2′-yl)-4,6-dimethoxypyrimidine-5-carboxamide (70 mg, 0.143 mmol, 1.0equiv.) in DCE (2.5 mL) was added acetone (166.0 mg, 2.86 mmol, 20.0 equiv.) and AcOH (14.0 mg, 0.143 mmol, 1equiv.) at room temperature under argon atmosphere. The reaction mixture was stirred for 1 h at 50° C. To the above solution was added NaBH(OAc)₃ (157.0 mg, 0.72 mmol, 5.0 equiv.) at room temperature. The reaction mixture was stirred for 16 hours at room temperature. The reaction mixture was concentrated under vacuum, then diluted with ACN (1.0 mL). The crude product was purified by reversed-phase chromatography (Column: 40 g RP18 Column, Mobile Phase A: Water (0.1% FA), Mobile Phase B: ACN; Flow rate: 30 mL/min; Gradient: 0% B to 45% B in 25 min; RT: 18 min) to afford the title compound (48.0 mg, 63% yield) as a yellow solid. ESI-MS m/z=532.11 [M+H]⁺; Calculated MW: 531.06; ¹H NMR (400 MHz, Chloroform-d) δ 11.12 (s, 1H), 8.31 (s, 1H), 7.31 (d, J=8.1 Hz, 1H), 7.12 (d, J=1.8 Hz, 1H), 7.00 (dd, J=8.1, 1.9 Hz, 1H), 3.98 (s, 6H), 3.57 (s, 4H), 2.44 (s, 1H), 0.99 (d, J=6.2 Hz, 6H).

### Example 147:4-(2-(dimethylamino)ethoxy)-N—(7-isopropylspiro[chromeno[4,3-d]thiazole-4,3′-oxetan]-2-yl)-6-methoxypyrimidine-5-carboxamide

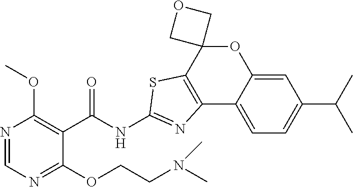 [SMILES: COc1ncnc(OCCN(C)C)c1C(=O)Nc1nc2c(s1)C1(COC1)Oc1cc(C(C)C)ccc1-2]

### Example 147A: 4-chloro-N—(7-isopropylspiro[chromeno[4,3-d]thiazole-4,3′-oxetan]-2-yl)-6-methoxypyrimidine-5-carboxamide

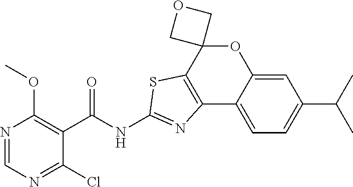 [SMILES: COc1ncnc(Cl)c1C(=O)Nc1nc2c(s1)C1(COC1)Oc1cc(C(C)C)ccc1-2]

To a stirred solution of 4-chloro-6-methoxypyrimidine-5-carboxylic acid (49 mg, 0.260 mmol, 1.50 equiv.) and NMI (50 mg, 0.608 mmol, 3.50 equiv.) in ACN (1 mL) was added TCFH (98 mg, 0.347 mmol, 2.00 equiv.) in ACN (1 mL) at 0° C. and it was stirred for 30 min. Then 7-isopropylspiro[chromeno[4,3-d]thiazole-4,3′-oxetan]-2-amine (50 mg, 0.174 mmol, 1.00 equiv.) was added at 0° C. and the mixture was stirred overnight at 60° C. The mixture was quenched with NaHCO₃(aq), then the resulting mixture was extracted with EA (2×10 mL). The combined organic layers were washed with brine (1×10 mL), dried over anhydrous Na₂SO₄. After filtration, the filtrate was concentrated under reduced pressure. The crude product was purified by prep-TLC (PE:EA=3:1) to afford the title compound (25 mg, 31%) as a yellow solid. ESI-MS m/z=459.11 [M+H]⁺; Calculated MW: 458.08.

### Example 147B: 4-(2-(dimethylamino)ethoxy)-N—(7-isopropylspiro[chromeno[4,3-d]thiazole-4,3′-oxetan]-2-yl)-6-methoxypyrimidine-5-carboxamide

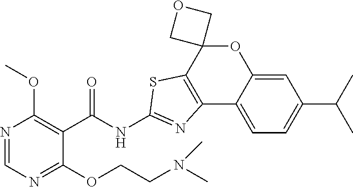 [SMILES: COc1ncnc(OCCN(C)C)c1C(=O)Nc1nc2c(s1)C1(COC1)Oc1cc(C(C)C)ccc1-2]

To the mixture of 2-(dimethylamino)ethan-1-ol (6 mg, 0.066 mmol, 1.20 equiv.) in THF (0.5 mL) was added 60% NaH (7 mg, 0.164 mmol, 3.00 equiv.) at 0° C., and the mixture was stirred for 30 min at room temperature. 4-chloro-N—(7-isopropylspiro[chromeno[4,3-d]thiazole-4,3′-oxetan]-2-yl)-6-methoxypyrimidine-5-carboxamide (25 mg, 0.0546 mmol, 1.00 equiv.) was added at 0° C. and the reaction was stirred overnight at room temperature. The mixture was quenched with ice water and the resulting mixture was extracted with EA (2×15 mL). The combined organic layers were washed with brine (1×10 mL), dried over anhydrous Na₂SO₄. After filtration, the filtrate was concentrated under reduced pressure. The crude product was purified by prep-TLC (DCM:MeOH=10:1) to afford the title compound (2.8 mg, 10%) as a white solid. ESI-MS m/z=512.30 [M+H]⁺; Calculated MW: 511.19. ¹H NMR (400 MHz, Chloroform-d) δ 8.49 (s, 1H), 7.55 (d, J=7.8 Hz, 1H), 6.95-6.81 (m, 2H), 5.08 (dd, J=7.1, 0.9 Hz, 2H), 4.91 (dd, J=7.1, 1.0 Hz, 2H), 4.78 (t, J=5.2 Hz, 2H), 4.09 (s, 3H), 2.87 (hept, J=6.8 Hz, 3H), 1.29-1.18 (m, 12H).

### Example 148: N—(7′-cyano-1-isopropylspiro[azetidine-3,4′-chromeno[4,3-d]thiazol]-2′-yl)-4,6-dimethoxypyrimidine-5-carboxamide

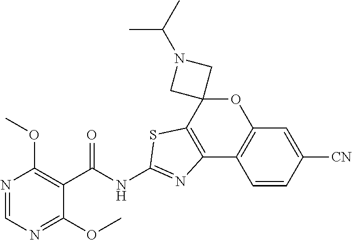 [SMILES: COc1ncnc(OC)c1C(=O)Nc1nc2c(s1)C1(CN(C(C)C)C1)Oc1cc(C#N)ccc1-2]

### Example 148A: tert-butyl 7′-cyano-2′-(4,6-dimethoxypyrimidine-5-carboxamido)spiro[azetidine-3,4′-chromeno[4,3-d]thiazole]-1-carboxylate

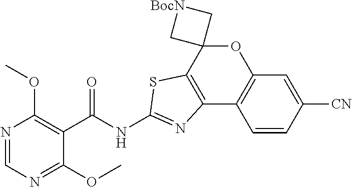 [SMILES: COc1ncnc(OC)c1C(=O)Nc1nc2c(s1)C1(CN(C(=O)OC(C)(C)C)C1)Oc1cc(C#N)ccc1-2]

Into a 10 mL round-bottom flask were added tert-butyl 7′-bromo-2′-(4,6-dimethoxypyrimidine-5-carboxamido)spiro[azetidine-3,4′-chromeno[4,3-d]thiazole]-1-carboxylate (60.0 mg, 0.10 mmol, 1.00 equiv.), THF (1.00 mL), H₂O (1.00 mL), Zn(CN)₂ (36.0 mg, 0.30 mmol, 3.00 equiv.) and t-BuXPhos Pd G3 (24.0 mg, 0.031 mmol, 0.30 equiv.) at room temperature. The resulting mixture was stirred for 3 h at 60° C. under argon atmosphere, cooled down to room temperature and diluted with water. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by prep-TLC (PE/EtOAc (2:1)) to afford the title compound (45.0 mg, 82.4% yield) as a yellow solid. ESI-MS m/z=537.20 [M+H]⁺; Calculated MW: 536.15.

### Example 148B: N—(7′-cyanospiro[azetidine-3,4′-chromeno[4,3-d]thiazol]-2′-yl)-4,6-dimethoxypyrimidine-5-carboxamide

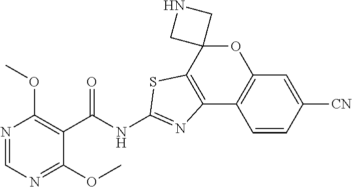 [SMILES: COc1ncnc(OC)c1C(=O)Nc1nc2c(s1)C1(CNC1)Oc1cc(C#N)ccc1-2]

To a stirred solution of tert-butyl 7′-cyano-2′-(4,6-dimethoxypyrimidine-5-carboxamido)spiro[azetidine-3,4′-chromeno[4,3-d]thiazole]-1-carboxylate (35.0 mg, 0.065 mmol, 1.00 equiv.) in DCM (1.50 mL) was added TFA (0.5 mL) in portions at 0° C. The resulting mixture was stirred for 2 h at room temperature. The reaction was diluted with toluene (2.0 mL), then concentrated under reduced pressure. This resulted in the title compound (45.0 mg, TFA salt, crude) as a yellow solid. ESI-MS m/z=437.10 [M+H]⁺; Calculated MW: 436.10.

### Example 148C: N—(7′-cyano-1-isopropylspiro[azetidine-3,4′-chromeno[4,3-d]thiazol]-2′-yl)-4,6-dimethoxypyrimidine-5-carboxamide

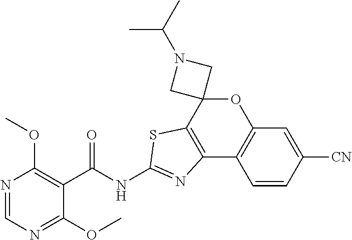 [SMILES: COc1ncnc(OC)c1C(=O)Nc1nc2c(s1)C1(CN(C(C)C)C1)Oc1cc(C#N)ccc1-2]

To a stirred solution of N—(7′-cyanospiro[azetidine-3,4′-chromeno[4,3-d]thiazol]-2′-yl)-4,6-dimethoxypyrimidine-5-carboxamide (30.0 mg, 0.069 mmol, 1.0equiv.) in DCE (1.5 mL) was added acetone (80.0 mg, 1.37 mmol, 20.0 equiv.) and AcOH (7.0 mg, 0.069 mmol, 1equiv.) at room temperature under argon atmosphere. The reaction mixture was stirred for 1 h at 50° C. To the above solution was added NaBH(OAc)₃ (157.0 mg, 0.72 mmol, 5.0 equiv.) at room temperature. The reaction mixture was stirred for 16 hours at room temperature. The reaction mixture was concentrated under vacuum, then diluted with ACN (1.0 mL). The crude product was purified by reversed-phase chromatography (Column: 40 g RP18 Column, Mobile Phase A: Water (0.1% FA), Mobile Phase B: ACN; Flow rate: 30 mL/min; Gradient: 0% B to 45% B in 25 min; RT: 15 min) to afford the title compound (15.0 mg, 45% yield) as a yellow solid. ESI-MS m/z=479.20 [M+H]⁺; Calculated MW: 478.14; ¹H NMR (400 MHz, Chloroform-d) δ 10.67 (s, 1H), 8.42 (s, 1H), 7.61 (d, J=7.7 Hz, 1H), 7.23-7.17 (m, 2H), 4.07 (s, 6H), 3.66-3.53 (m, 4H), 2.46 (p, J=6.2 Hz, 1H), 0.99 (d, J=6.2 Hz, 6H).

### Example 149: N—(1-cyclopropyl-7′-(trifluoromethyl)spiro[azetidine-3,4′-chromeno[4,3-d]thiazol]-2′-yl)-4,6-dimethoxypyrimidine-5-carboxamide

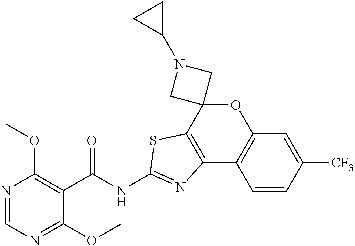 [SMILES: COc1ncnc(OC)c1C(=O)Nc1nc2c(s1)C1(CN(C3CC3)C1)Oc1cc(C(F)(F)F)ccc1-2]

To a mixture of 4,6-dimethoxy-N—(7′-(trifluoromethyl)spiro[azetidine-3,4′-chromeno[4,3-d]thiazol]-2′-yl)pyrimidine-5-carboxamide (50 mg, 104.29 μmol, 1.0 equiv.) and cyclopropyl boronic acid (9.9 mg, 114.72 μmol, 1.1 equiv.) in 1,2-dichloroethane (1.0 mL) was added Na₂CO₃ (22.11 mg, 208.6 umol, 2.0 equiv.), Cu(OAc)₂ (18.9 mg, 104.29 μmol, 1.0 equiv.) and 2,2′-bipyridine (16.3 mg, 104.29 mmol, 1.0 equiv.) at 25° C. The mixture was stirred at 70° C. under O₂ (15 psi) for 2 hours. The mixture was cooled to room temperature and was filtered and washed with ethyl acetate (50 mL). After concentration, the residue was quenched with ice water (10 mL) and sat. NH₄Cl (10 mL). The aqueous phase was extracted with EA (10 mL×2). The combine organic layers were washed with brine (20 mL×2), dried over Na₂SO₄, filtered, and concentrated under reduce pressure. The crude product was purified by prep-TLC (PE:EA=2:1) to afford the title compound (12 mg, 22%) as white solid. ESI-MS m/z=520.16 [M+H]⁺; Calculated MW: 519.49. ¹H NMR (400 MHz, DMSO-d₆) δ 12.90 (s, 1H), 8.59 (s, 1H), 7.68 (d, J=7.8 Hz, 1H), 7.38-7.32 (m, 1H), 7.26 (d, J=1.7 Hz, 1H), 3.92 (s, 6H), 3.68-3.56 (m, 4H), 2.04-1.96 (m, 1H), 0.45-0.26 (m, 4H).

### Example 150: N—(1-isobutyl-7′-(trifluoromethyl)spiro[azetidine-3,4′-chromeno[4,3-d]thiazol]-2′-yl)-4,6-dimethoxypyrimidine-5-carboxamide

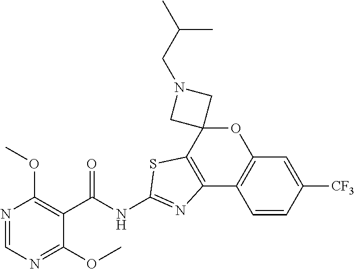 [SMILES: COc1ncnc(OC)c1C(=O)Nc1nc2c(s1)C1(CN(CC(C)C)C1)Oc1cc(C(F)(F)F)ccc1-2]

4,6-dimethoxy-N—(7′-(trifluoromethyl)spiro[azetidine-3,4′-chromeno[4,3-d]thiazol]-2′-yl)pyrimidine-5-carboxamide (50 mg, 104.29 μmol, 1 equiv.) and DEC (1 mL) were added to a flame-dried flask. Isobutyraldehyde (47.6 μl, 37.6 mg, 521.45 μmol, 5equiv.) was diluted with DCE (0.2 mL) and then added to the mixture at rt. AcOH (11.9 μl, 12.5 mg, 208.58 μmol, 2 equiv.) was diluted with DCE (0.2 mL) and then added to the mixture at rt. The reaction mixture was stirred at rt for 15 minutes. NaBH(OAc)₃ (66.3 mg, 312.87 μmol, 3 equiv.) was added to the mixture at 0° C. The reaction mixture was stirred at rt for 1 hour. The mixture was quenched with ice-water (5 mL) and NH₄Cl sat aqueous (5 mL). The aqueous layer was extracted with EA (20 mL×2). The combined organic layers were washed with brine (20 mL×2), dried over Na₂SO₄, filtrated, and concentrated under reduced pressure. The residue was purified by prep-TLC (PE:EA=2:1) to obtain the desired product N—(1-isobutyl-7′-(trifluoromethyl)spiro[azetidine-3,4′-chromeno[4,3-d]thiazol]-2′-yl)-4,6-dimethoxypyrimidine-5-carboxamide (33 mg, 59%) as white solid. ESI-MS m/z=536.19 [M+H]⁺; Calculated MW: 535.54. ¹H NMR (400 MHz, DMSO-d₆) δ 12.89 (s, 1H), 8.59 (s, 1H), 7.68 (d, J=7.9 Hz, 1H), 7.33 (dd, J=8.0, 1.8 Hz, 1H), 7.24 (d, J=1.7 Hz, 1H), 3.93 (s, 6H), 3.67-3.60 (m, 2H), 3.39-3.32 (m, 2H), 2.30 (d, J=7.1 Hz, 2H), 1.55 (dq, J=13.4, 6.7 Hz, 1H), 0.87 (d, J=6.6 Hz, 6H).

### Example 151:4-(2-(dimethylamino)ethoxy)-6-methoxy-N—(7-(trifluoromethyl)spiro[chromeno[4,3-d]thiazole-4,3′-oxetan]-2-yl)pyrimidine-5-carboxamide

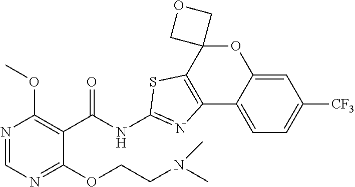 [SMILES: COc1ncnc(OCCN(C)C)c1C(=O)Nc1nc2c(s1)C1(COC1)Oc1cc(C(F)(F)F)ccc1-2]

### Example 151A: 4-chloro-6-methoxy-N—(7-(trifluoromethyl)spiro[chromeno[4,3-d]thiazole-4,3′-oxetan]-2-yl)pyrimidine-5-carboxamide

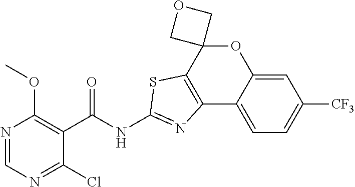 [SMILES: COc1ncnc(Cl)c1C(=O)Nc1nc2c(s1)C1(COC1)Oc1cc(C(F)(F)F)ccc1-2]

To a stirred solution of 4-chloro-6-methoxypyrimidine-5-carboxylic acid (54 mg, 0.287 mmol, 1.50 equiv.) and NMI (55 mg, 0.669 mmol, 3.50 equiv.) in ACN (1 mL) was added TCFH (107 mg, 0.382 mmol, 2.00 equiv.) at 0° C. and the reaction was stirred for 30 min. Then 7-(trifluoromethyl)spiro[chromeno[4,3-d]thiazole-4,3′-oxetan]-2-amine (60 mg, 0.191 mmol, 1.00 equiv.) in ACN (0.5 mL) was added at 0° C. and the mixture was stirred overnight at 60° C. The mixture was quenched with NaHCO₃(aq) and extracted with EA (2×10 mL). The combined organic layers were washed with brine (1×10 mL), dried over anhydrous Na₂SO₄. After filtration, the filtrate was concentrated under reduced pressure. The crude product was purified by prep-TLC (PE:EA=4:1) to afford the title compound (40 mg, 43%) as a white solid. ESI-MS m/z=485.03 [M+H]⁺; Calculated MW: 484.02.

### Example 151B: 4-(2-(dimethylamino)ethoxy)-6-methoxy-N—(7-(trifluoromethyl)spiro[chromeno[4,3-d]thiazole-4,3′-oxetan]-2-yl)pyrimidine-5-carboxamide

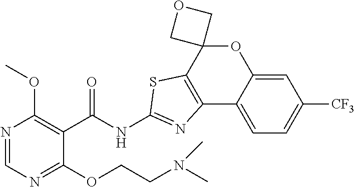 [SMILES: COc1ncnc(OCCN(C)C)c1C(=O)Nc1nc2c(s1)C1(COC1)Oc1cc(C(F)(F)F)ccc1-2]

To the mixture of 2-(dimethylamino)ethan-1-ol (9 mg, 0.099 mmol, 1.20 equiv.) in THF (1 mL) was added 60% NaH (10 mg, 0.248 mmol, 3.00 equiv.) at 0° C., and the mixture was stirred for 30 min at room temperature. 4-chloro-6-methoxy-N—(7-(trifluoromethyl)spiro[chromeno[4,3-d]thiazole-4,3′-oxetan]-2-yl)pyrimidine-5-carboxamide (40 mg, 0.0826 mmol, 1.00 equiv.) was added at 0° C. and it was stirred overnight at room temperature. The mixture was quenched with ice water, then the resulting mixture was extracted with EA (2×15 mL). The combined organic layers were washed with brine (1×10 mL), dried over anhydrous Na₂SO₄. After filtration, the filtrate was concentrated under reduced pressure. The crude product was purified by prep-TLC (DCM:MeOH=15:1) to afford the title compound (20 mg, 45%) as a white solid. ESI-MS m/z=538.25 [M+H]⁺; Calculated MW: 537.13. ¹H NMR (400 MHz, Chloroform-d) δ 8.51 (s, 1H), 7.69 (d, J=7.8 Hz, 1H), 7.26 (s, 2H), 5.10 (dd, J=7.2, 1.0 Hz, 2H), 4.97-4.89 (m, 2H), 4.80-4.73 (m, 2H), 4.10 (s, 3H), 4.04 (s, 0H), 3.51 (s, 0H), 2.69 (s, 2H), 2.50 (s, 6H).

### Example 152: N—(1′-isopropyl-7-(trifluoromethyl)spiro[chromeno[4,3-d]thiazole-4,4′-piperidin]-2-yl)-4,6-dimethoxypyrimidine-5-carboxamide

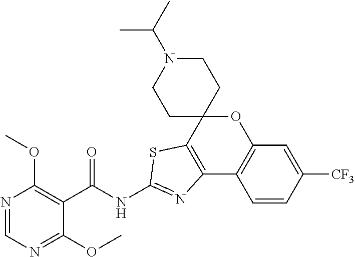 [SMILES: COc1ncnc(OC)c1C(=O)Nc1nc2c(s1)C1(CCN(C(C)C)CC1)Oc1cc(C(F)(F)F)ccc1-2]

4,6-dimethoxy-N—(7-(trifluoromethyl)spiro[chromeno[4,3-d]thiazole-4,4′-piperidin]-2-yl)pyrimidine-5-carboxamide (50 mg, 98.52 μmol, 1 equiv.) and DEC (1 mL) were added to a flame-dried flask. Acetone (36.3 μl, 28.61 mg, 492.62 μmol, 5equiv.) was diluted with DCE (0.2 mL) and then added to the mixture at rt. AcOH (11.27 μl, 11.83 mg, 197.05 μmol, 2 equiv.) was diluted with DCE (0.1 mL) and then added to the mixture at rt. The reaction mixture was stirred at rt for 15 minutes. NaBH(OAc)₃ (62.6 mg, 295.57 μmol, 3 equiv.) was added to the mixture at 0° C. The reaction mixture was stirred at rt for 16 hours. After concentration, the residue was purified by prep-TLC (DCM:MeOH=10:1) to get the desire product N—(1′-isopropyl-7-(trifluoromethyl)spiro[chromeno[4,3-d]thiazole-4,4′-piperidin]-2-yl)-4,6-dimethoxypyrimidine-5-carboxamide (10 mg, 18%) as white solid. ESI-MS m/z=550.24 [M+H]⁺; Calculated MW: 549.56. ¹H NMR (400 MHz, DMSO-d₆) δ 12.95 (s, 1H), 8.63 (s, 1H), 7.75 (d, J=8.0 Hz, 1H), 7.40 (d, J=7.6 Hz, 2H), 3.96 (s, 6H), 3.25-2.30 (m, 5H), 2.29-1.95 (m, 4H), 1.11 (s, 6H).

### Example 153: 4,6-dimethoxy-N—(1-(1-methoxycyclopropyl)-7′-(trifluoromethyl)spiro[azetidine-3,4′-chromeno[4,3-d]thiazol]-2′-yl)pyrimidine-5-carboxamide

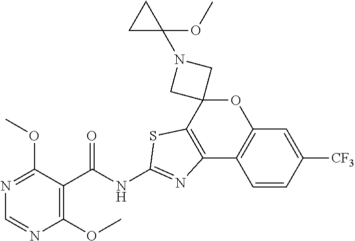 [SMILES: COc1ncnc(OC)c1C(=O)Nc1nc2c(s1)C1(CN(C3(OC)CC3)C1)Oc1cc(C(F)(F)F)ccc1-2]

A solution of 4,6-dimethoxy-N—(7′-(trifluoromethyl)spiro[azetidine-3,4′-chromeno[4,3-d]thiazol]-2′-yl)pyrimidine-5-carboxamide (30 mg, 62.57 μmol, 1equiv.) and (1-ethoxycyclopropoxy) trimethylsilane (21.8 mg, 25.16 μl, 125.15 μmol, 2.0 equiv.) in MeOH (0.3 mL)/THF (0.3 mL) was treated with NaBH₃CN (5.9 mg, 93.86 μmol, 1.5 equiv.) and acetic acid (19.0 mg, 17.89 μl, 321.87 μmol, 5 equiv.) at 60° C. for 16 h. The mixture was quenched with ice-water (5 mL). The aqueous was extracted with EA (10 mL×2). The combine organic layers were washed with brine (20 mL×2), dried over Na₂SO₄, filtered, and concentrated under reduce pressure. The crude product was purified by prep-TLC (PE:EA=1:1) to afford the title compound (10 mg, 29%) as white solid. ESI-MS m/z=550.19 [M+H]⁺; Calculated MW: 549.50. ¹H NMR (400 MHz, Chloroform-d) δ 10.72 (s, 1H), 8.39 (s, 1H), 7.63 (dd, J=8.2, 1.3 Hz, 1H), 7.18-7.12 (m, 2H), 4.05 (s, 6H), 3.77-3.64 (m, 4H), 3.46 (s, 3H), 0.92-0.75 (m, 4H).

### Example 154: N-(l′-cyclopropyl-7-(trifluoromethyl)spiro[chromeno[4,3-d]thiazole-4,4′-piperidin]-2-yl)-4,6-dimethoxypyrimidine-5-carboxamide

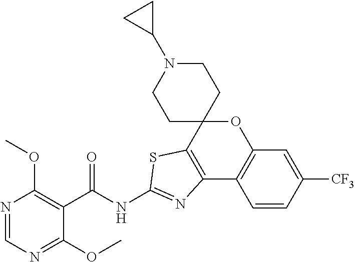 [SMILES: COc1ncnc(OC)c1C(=O)Nc1nc2c(s1)C1(CCN(C3CC3)CC1)Oc1cc(C(F)(F)F)ccc1-2]

To a mixture of 4,6-dimethoxy-N—(7-(trifluoromethyl)spiro[chromeno[4,3-d]thiazole-4,4′-piperidin]-2-yl)pyrimidine-5-carboxamide (50 mg, 98.52 μmol, 1.0 equiv.) and cyclopropyl-boronic acid (9.3 mg, 108.38 μmol, 1.1 equiv.) in 1,2-dichloroethane (1.0 mL) was added Na₂CO₃ (20.9 mg, 197.05 umol, 2.0 equiv.), Cu(OAc)₂ (17.9 mg, 98.52 μmol, 1.0 equiv.) and 2,2′-bipyridine (15.4 mg, 98.52 mmol, 1.0 equiv.) at 25° C. The mixture was stirred at 70° C. under O₂ (15 psi) for 2 hours. The mixture was cooled to room temperature and was filtered and washed with ethyl acetate (50 mL). The mixture was concentrated under reduce pressure. The residue was quenched with ice water (10 mL) and sat. NH₄Cl (10 mL). The aqueous phase was extracted with EA (10 mL×2). The combine organic layers were washed with brine (20 mL×2), dried over Na₂SO₄, filtrated, and concentrated under reduce pressure. The crude product was purified by prep-TLC (DCM:MeOH=20:1) to afford the title compound (10 mg, 19%) as white solid. ESI-MS m/z=548.21 [M+H]⁺; Calculated MW: 547.55. ¹H NMR (400 MHz, DMSO-d₆) δ 12.89 (s, 1H), 8.58 (s, 1H), 7.71 (d, J=7.9 Hz, 1H), 7.38-7.30 (m, 2H), 3.92 (d, J=1.2 Hz, 6H), 2.83-2.63 (m, 4H), 2.10 (d, J=13.1 Hz, 2H), 1.95-1.80 (m, 2H), 1.73 (td, J=6.5, 3.3 Hz, 1H), 0.42 (dd, J=6.4, 2.1 Hz, 2H), 0.30 (p, J=3.9 Hz, 2H).

### Example 155: N—(1′-isobutyl-7-(trifluoromethyl)spiro[chromeno[4,3-d]thiazole-4,4′-piperidin]-2-yl)-4,6-dimethoxypyrimidine-5-carboxamide

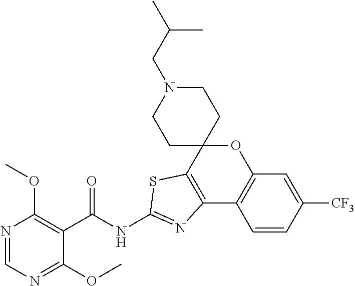 [SMILES: COc1ncnc(OC)c1C(=O)Nc1nc2c(s1)C1(CCN(CC(C)C)CC1)Oc1cc(C(F)(F)F)ccc1-2]

4,6-dimethoxy-N—(7-(trifluoromethyl)spiro[chromeno[4,3-d]thiazole-4,4′-piperidin]-2-yl)pyrimidine-5-carboxamide (30 mg, 59.11 μmol, 1 equiv.) and DEC (0.6 mL) were added to a flame-dried flask. Isobutyraldehyde (27.0 μl, 21.31 mg, 295.57 μmol, 5equiv.) was diluted with DCE (0.1 mL) and then added to the mixture at rt. AcOH (6.8 μl, 7.1 mg, 118.23 μmol, 2 equiv.) was diluted with DCE (0.1 mL) and then added to the mixture at rt. The reaction mixture was stirred at rt for 15 minutes. NaBH(OAc)₃ (37.6 mg, 117.34 μmol, 3 equiv.) was added to the mixture at 0° C. The reaction mixture was stirred at rt for 1 hour and quenched with ice-water (5 mL) and sat. NH₄Cl (5 mL). The aqueous layer was extracted with EA (20 mL×2). The combined organic layers were washed with brine (20 mL×2), dried over Na₂SO₄, filtrated, and concentrated under reduced pressure. The residue was purified by prep-TLC (PE:EA=2:1) to produce the desire product N—(1′-isobutyl-7-(trifluoromethyl)spiro[chromeno[4,3-d]thiazole-4,4′-piperidin]-2-yl)-4,6-dimethoxypyrimidine-5-carboxamide (5 mg, 15%) as white solid. ESI-MS m/z=564.25 [M+H]⁺; Calculated MW: 563.59. ¹H NMR (400 MHz, DMSO-d₆) δ 12.90 (s, 1H), 8.58 (s, 1H), 7.71 (d, J=7.9 Hz, 1H), 7.35 (d, J=8.0 Hz, 1H), 7.28 (d, J=1.7 Hz, 1H), 3.92 (s, 6H), 2.64 (d, J=12.1 Hz, 2H), 2.40 (d, J=10.3 Hz, 2H), 2.11 (d, J=8.0 Hz, 4H), 2.00-1.85 (m, 2H), 1.76 (dt, J=13.4, 6.7 Hz, 1H), 0.85 (d, J=6.5 Hz, 6H).

### Example 156: tert-butyl 2′-(4,6-dimethoxypyrimidine-5-carboxamido)spiro[azetidine-3,4′-thiazolo[4′,5′: 4,5]pyrano[2,3-c]pyridine]-1-carboxylate

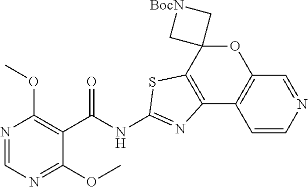 [SMILES: COc1ncnc(OC)c1C(=O)Nc1nc2c(s1)C1(CN(C(=O)OC(C)(C)C)C1)Oc1cnccc1-2]

### Example 156A: tert-butyl 4′-oxo-3′,4′-dihydrospiro[azetidine-3,2′-pyrano[2,3-c]pyridine]-1-carboxylate

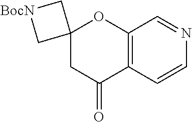 [SMILES: CC(C)(C)OC(=O)N1CC2(CC(=O)c3ccncc3O2)C1]

To a stirred solution of 1-(3-hydroxypyridin-4-yl)ethan-1-one (70 mg, 510.43 μmol, 1.0 equiv.) in ACN (1.4 mL) was added tert-butyl 3-oxoazetidine-1-carboxylate (131.1 mg, 765.65 μmol, 1.5 equiv.), pyrrolidine (108.9 mg, 0.127 mL, 1.53 mmol, 3.0 equiv.) and AcOH (92.0 mg, 87.66 μl, 1.53 mol, 3 equiv.) at rt. The mixture was stirred at 65° C. for 4 hours. Then the mixture was concentrated under vacuum. The crude product was purified by prep-TLC (PE:EA=1:1) to afford the title compound (43 mg, 29%) as yellow solid. ¹H NMR (400 MHz, Chloroform-d) δ 8.59 (s, 1H), 8.38 (d, J=5.0 Hz, 1H), 7.61 (dd, J=5.0, 0.8 Hz, 1H), 4.15-4.09 (m, 2H), 3.97 (dd, J=9.6, 1.1 Hz, 2H), 3.10 (s, 2H), 1.43 (s, 9H).

### Example 156B: tert-butyl 2′-aminospiro[azetidine-3,4′-thiazolo[4′,5′: 4,5]pyrano[2,3-c]pyridine]-1-carboxylate

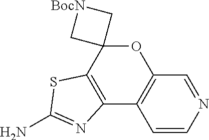 [SMILES: CC(C)(C)OC(=O)N1CC2(C1)Oc1cnccc1-c1nc(N)sc12]

To a stirred solution of ethyl tert-butyl 4′-oxo-3′,4′-dihydrospiro[azetidine-3,2′-pyrano[2,3-c]pyridine]-1-carboxylate tert-butyl 4′-oxo-3′,4′-dihydrospiro[azetidine-3,2′-pyrano[2,3-c]pyridine]-1-carboxylate (51 mg, 175.67 μmol, 1.0 equiv.) in toluene (3 mL) was added TsOH·H₂O (6.7 mg, 35.13 umol, 0.2 equiv.) and pyrrolidine (62.5 mg, 73.32 μl, 878.34 μmol, 5.0 equiv.) at room temperature. The reaction mixture was stirred at 120° C. for 2 hours and concentrated. The resulting mixture was dissolved in methanol (2 mL) and sulfur (14.1 mg, 439.17 μmol, 2.5 equiv.) was added. The mixture was stirred for 10 minutes at room temperature then cyanamide (36.9 mg, 439.17 μmol, 2.5 equiv.) was added. The mixture was stirred overnight at room temperature and concentrated. The resulting residue was extracted with ethyl acetate (2×20 mL). The combined organic layers were washed with brine (2*30 mL), dried over anhydrous Na₂SO₄, filtrated, and concentrated under vacuum. The crude product was purified by prep-TLC (DCM:MeOH=15:1) to afford the title compound (13 mg, 21%) as yellow solid. ¹H NMR (400 MHz, Chloroform-d) δ 8.45-8.14 (m, 2H), 7.41 (d, J=4.6 Hz, 1H), 5.29 (s, 2H), 4.41 (dd, J=9.8, 3.5 Hz, 2H), 4.18 (dd, J=9.9, 3.5 Hz, 2H), 1.47 (d, J=3.6 Hz, 9H).

### Example 156C: tert-butyl 2′-(4,6-dimethoxypyrimidine-5-carboxamido)spiro[azetidine-3,4′-thiazolo[4′,5′: 4,5]pyrano[2,3-c]pyridine]-1-carboxylate

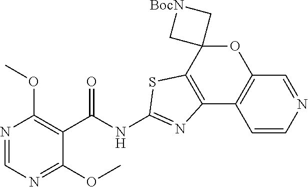 [SMILES: COc1ncnc(OC)c1C(=O)Nc1nc2c(s1)C1(CN(C(=O)OC(C)(C)C)C1)Oc1cnccc1-2]

Tert-butyl 2′-aminospiro[azetidine-3,4′-thiazolo[4′,5′: 4,5]pyrano[2,3-c]pyridine]-1-carboxylate (13 mg, 37.53 μmol, 1 equiv.) and 4,6-dimethoxypyrimidine-5-carboxylic acid (10.37 mg, 56.29 μmol, 1.5 equiv.) were added to a flame-dried three necked flask. The flask was evacuated and back-filled with Ar. ACN (0.2 mL) was added to the mixture and cool to 0° C. NMI (10.8 mg, 10.37 μl, 131.35 μmol, 3.5 equiv.) was added to the mixture at 0° C. TCFH (15.8 mg, 56.29 μmol, 1.5 equiv.) was dissolved in 0.1 mL ACN and added to the mixture at 0° C. The mixture was stirred at rt for 3 hours and at 60° C. for 16 hours. The mixture was concentrated under reduce pressure then quenched with ice-water (20 mL). The aqueous was extracted with EA (20 mL×2). The combine organic layers were washed with brine (20 mL×2), dried over Na₂SO₄, filtered, and concentrated under reduce pressure. The crude product was purified by prep-TLC (DCM:MeOH=10:1) to afford the title compound (2 mg, 10%) as yellow solid. ESI-MS m/z=513.21 [M+H]⁺; Calculated MW: 512.54. ¹H NMR (400 MHz, Chloroform-d) & 10.36 (s, 1H), 8.51 (s, 1H), 8.36-8.26 (m, 2H), 7.62 (d, J=4.8 Hz, 1H), 4.47 (d, J=9.8 Hz, 2H), 4.27 (d, J=9.8 Hz, 2H), 4.15 (s, 6H), 1.44 (s, 9H).

### Example 157: 4,6-dimethoxy-N—(1-(2-methoxyethyl)-7′-(trifluoromethyl)spiro[azetidine-3,4′-chromeno[4,3-d]thiazol]-2′-yl)pyrimidine-5-carboxamide

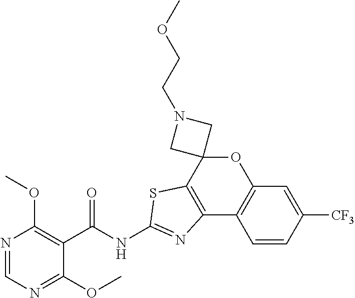 [SMILES: COCCN1CC2(C1)Oc1cc(C(F)(F)F)ccc1-c1nc(NC(=O)c3c(OC)ncnc3OC)sc12]

4,6-dimethoxy-N—(7′-(trifluoromethyl)spiro[azetidine-3,4′-chromeno[4,3-d]thiazol]-2′-yl)pyrimidine-5-carboxamide (50 mg, 104.29 μmol, 1 equiv.) was added to a solution of 2-methoxyacetaldehyde in DCE (2 mL). AcOH (11.9 μl, 12.5 mg, 208.58 μmol, 2 equiv.) was diluted with DCE (0.2 mL) and added to the mixture at rt. The reaction mixture was stirred at rt for 15 minutes. NaBH(OAc)₃ (66.3 mg, 312.87 μmol, 3 equiv.) was added to the mixture at 0° C. The reaction mixture was stirred at rt for 1 hour and quenched with ice-water (5 mL) and sat. NH₄Cl (5 mL). The aqueous layer was extracted with EA (20 mL×2). The combined organic layers were washed with brine (20 mL×2), dried over Na₂SO₄, filtered, and concentrated under reduce pressure. The residue was purified by prep-TLC (DCM:MeOH=50:1) to provide 4,6-dimethoxy-N—(1-(2-methoxyethyl)-7′-(trifluoromethyl)spiro[azetidine-3,4′-chromeno[4,3-d]thiazol]-2′-yl)pyrimidine-5-carboxamide (5 mg, 8.9%) as white solid. ESI-MS m/z=538.20 [M+H]⁺; Calculated MW: 537.51. ¹H NMR (400 MHz, DMSO-d₆) δ 12.93 (br, s, 1H), 8.59 (d, J=2.5 Hz, 1H), 7.70 (dd, J=15.9, 7.9 Hz, 1H), 7.37-7.31 (m, 1H), 7.25 (d, J=1.7 Hz, 1H), 3.93 (s, 6H), 3.70-3.63 (m, 2H), 3.48-3.43 (m, 2H), 3.36 (d, J=5.6 Hz, 2H), 3.22 (s, 3H), 2.67 (t, J=5.6 Hz, 2H).

### Example 158: N—(4,4-diethyl-7-(trifluoromethyl)-4H-chromeno[4,3-d]thiazol-2-yl)-2,4,6-trimethoxypyrimidine-5-carboxamide

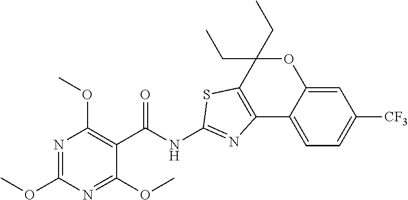 [SMILES: CCC1(CC)Oc2cc(C(F)(F)F)ccc2-c2nc(NC(=O)c3c(OC)nc(OC)nc3OC)sc21]

To a stirred solution of 2,4,6-trimethoxypyrimidine-5-carboxylic acid (21.0 mg, 0.100 mmol, 1.10 equiv.) and 4,4-diethyl-7-(trifluoromethyl)-4H-chromeno[4,3-d]thiazol-2-amine (30.0 mg, 0.091 mmol, 1.00 equiv.) in ACN (1.00 mL) was added NMI (26.00 mg, 0.320 mmol, 3.50 equiv.) and TCFH (39.00 mg, 0.139 mmol, 1.50 equiv.) in portions at 0° C. The resulting mixture was stirred for 16 h at 60° C. under argon atmosphere. The reaction was diluted with water (20 mL), extracted with EA (3×10 mL), washed with NaCl(aq.) and dried over anhydrous Na₂SO₄. After filtration, the filtrate was concentrated under reduced pressure. The residue was purified by prep-TLC (DCM/PE/EtOAc (7:4:1)) to afford the title compound (27.0 mg, 56.4% yield) as an off-white solid. ESI-MS m/z=525.19 [M+H]⁺; Calculated MW: 524.13; ¹H NMR (400 MHz, Chloroform-d) δ 10.96 (s, 1H), 7.63-7.58 (m, 1H), 7.11-7.06 (m, 2H), 4.03 (s, 6H), 3.97 (s, 3H), 2.01 (dq, J=14.7, 7.4 Hz, 2H), 1.89 (dq, J=14.6, 7.4 Hz, 2H), 0.98 (t, J=7.4 Hz, 6H).

### Example 159: N—(1-cyclobutyl-7′-(trifluoromethyl)spiro[azetidine-3,4′-chromeno[4,3-d]thiazol]-2′-yl)-4,6-dimethoxypyrimidine-5-carboxamide

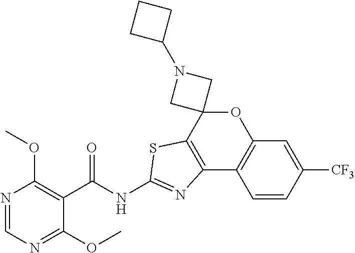 [SMILES: COc1ncnc(OC)c1C(=O)Nc1nc2c(s1)C1(CN(C3CCC3)C1)Oc1cc(C(F)(F)F)ccc1-2]

4,6-dimethoxy-N—(7′-(trifluoromethyl)spiro[azetidine-3,4′-chromeno[4,3-d]thiazol]-2′-yl)pyrimidine-5-carboxamide (50 mg, 104.29 μmol, 1 equiv.) and DEC (1 mL) were added to a flame-dried flask. Cyclobutanone (39.0 μl, 36.55 mg, 521.45 μmol, 5 equiv.) was diluted with DCE (0.2 mL) and added to the mixture at rt. AcOH (12.5 mg, 208.58 μmol, 2 equiv.) was diluted with DCE (0.2 mL) and added to the mixture at rt. The reaction mixture was stirred at rt for 15 minutes. NaBH(OAc)₃ (66.3 mg, 312.87 μmol, 3 equiv.) was added to the mixture at 0° C. The reaction mixture was stirred at rt for 1 hour. The mixture was quenched with ice-water (5 mL) and sat. NH₄Cl (5 mL). The aqueous layer was extracted with EA (20 mL×2). The combined organic layers were washed with brine (20 mL×2), dried over Na₂SO₄, filtrated, and concentrated under reduce pressure. The residue was purified by prep-TLC (PE:EA=2:1) to provide N—(1-cyclobutyl-7′-(trifluoromethyl)spiro[azetidine-3,4′-chromeno[4,3-d]thiazol]-2′-yl)-4,6-dimethoxypyrimidine-5-carboxamide (20 mg, 35.9%) as white solid. ESI-MS m/z=534.17 [M+H]⁺; Calculated MW: 533.52. ¹H NMR (400 MHz, DMSO-d₆) δ 12.91 (s, 1H), 8.59 (s, 1H), 7.73-7.65 (m, 1H), 7.37-7.31 (m, 1H), 7.26 (d, J=1.7 Hz, 1H), 3.93 (s, 6H), 3.60-3.50 (m, 2H), 3.48-3.39 (m, 2H), 3.26-3.18 (m, 1H), 1.91 (q, J=7.5, 5.5 Hz, 2H), 1.84-1.73 (m, 2H), 1.72-1.51 (m, 2H).

### Example 160: 4,6-dimethoxy-N—(1-(oxetan-3-yl)-7′-(trifluoromethyl)spiro[azetidine-3,4′-chromeno[4,3-d]thiazol]-2′-yl)pyrimidine-5-carboxamide

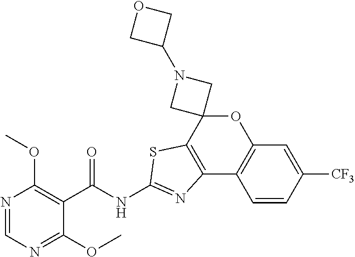 [SMILES: COc1ncnc(OC)c1C(=O)Nc1nc2c(s1)C1(CN(C3COC3)C1)Oc1cc(C(F)(F)F)ccc1-2]

4,6-dimethoxy-N—(7′-(trifluoromethyl)spiro[azetidine-3,4′-chromeno[4,3-d]thiazol]-2′-yl)pyrimidine-5-carboxamide (50 mg, 104.29 μmol, 1 equiv.) and DCE (1 mL) were added to a flame-dried flask. Oxetan-3-one (30.5 μl, 37.6 mg, 521.45 μmol, 5 equiv.) was diluted with DCE (0.2 mL) and added to the mixture at rt. AcOH (11.9 μl, 12.5 mg, 208.58 μmol, 2 equiv.) was diluted with DCE (0.2 mL) and added to the mixture at rt. The reaction mixture was stirred at rt for 15 minutes. NaBH(OAc)₃ (66.3 mg, 312.87 μmol, 3 equiv.) was added to the mixture at 0° C. The reaction mixture was stirred at rt for 1 hour. The mixture was quenched with ice-water (5 mL) and sat. NH₄Cl (5 mL). The aqueous layer was extracted with EA (20 mL×2). The combined organic layers were washed with brine (20 mL×2), dried over Na₂SO₄, filtered, and concentrated under reduce pressure. The residue was purified by prep-TLC (PE:EA:DCM=1:1:1) to produce 4,6-dimethoxy-N—(1-(oxetan-3-yl)-7′-(trifluoromethyl)spiro[azetidine-3,4′-chromeno[4,3-d]thiazol]-2′-yl)pyrimidine-5-carboxamide (20 mg, 35.8%) as white solid. ESI-MS m/z=536.17 [M+H]⁺; Calculated MW: 535.49. ¹H NMR (400 MHz, DMSO-d₆) δ 12.92 (s, 1H), 8.60 (s, 1H), 7.70 (d, J=7.8 Hz, 1H), 7.38-7.31 (m, 1H), 7.27 (d, J=1.7 Hz, 1H), 4.59 (t, J=6.7 Hz, 2H), 4.39 (dd, J=6.7, 5.2 Hz, 2H), 3.93 (s, 6H), 3.86 (ddd, J=11.8, 6.6, 5.1 Hz, 1H), 3.72-3.66 (m, 2H), 3.61-3.52 (m, 2H).

### Example 161:5-bromo-N—(4,4-diethyl-7-(trifluoromethyl)-4H-chromeno[4,3-d]thiazol-2-yl)-3-methoxy-1-methyl-1H-pyrazole-4-carboxamide

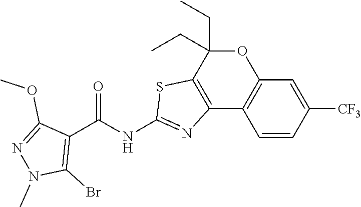 [SMILES: CCC1(CC)Oc2cc(C(F)(F)F)ccc2-c2nc(NC(=O)c3c(OC)nn(C)c3Br)sc21]

### Example 161A: ethyl 3,5-dibromo-1H-pyrazole-4-carboxylate

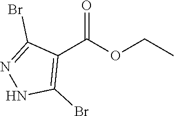

To a stirred solution of ethyl 1H-pyrazole-4-carboxylate (1.0 g, 7.14 mmol, 1.00 equiv.) and NaOAc (3.89 g, 28.6 mmol, 4.00 equiv.) in EtOH (6.00 mL)/H₂O (9.00 mL) was added Br₂ (2.86 g, 17.8 mmol, 2.50 equiv.) dropwise at 0° C. The resulting mixture was stirred for 16 h at room temperature under argon atmosphere and Na₂S₂O₃ (2.26 g, 14.3 mmol, 2.00 equiv.) was added in portions at 0° C. The resulting mixture was stirred for additional 0.5 h at room temperature. The reaction mixture was concentrated under reduced pressure. The residue was diluted with water, extracted with EtOAc (3×60 mL). The combined organic layers were washed with brine (2×50 mL), dried over anhydrous Na₂SO₄. After filtration, the filtrate was concentrated under reduced pressure. This resulted in the title compound (2.0 g, 95.2% yield) as an off-white solid. ESI-MS m/z=296.83 [M+H]⁺; Calculated MW: 295.88.

### Example 161B: ethyl 3,5-dibromo-1-methyl-1H-pyrazole-4-carboxylate

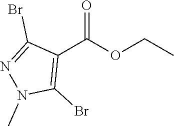

To a stirred solution of ethyl 3,5-dibromo-1H-pyrazole-4-carboxylate (500.0 mg, 1.69 mmol, 1.00 equiv.) and K₂CO₃ (467.0 mg, 3.38 mmol, 2.00 equiv.) in MeCN (5.00 mL) was added Mel (480.0 mg, 3.38 mmol, 2.00 equiv.) dropwise at room temperature. The resulting mixture was stirred for 16 h at 80° C. under argon atmosphere. The residue was diluted with water, extracted with EtOAc (3×40 mL). The combined organic layers were washed with brine (2×30 mL), dried over anhydrous Na₂SO₄. After filtration, the filtrate was concentrated under reduced pressure to produce the title compound (350.0 mg, crude) as a yellow solid. ESI-MS m/z=310.88 [M+H]⁺; Calculated MW: 309.90.

### Example 161C: 3,5-dibromo-1-methyl-1H-pyrazole-4-carboxylic acid

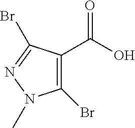

To a stirred solution of ethyl 3,5-dibromo-1-methyl-1H-pyrazole-4-carboxylate (100.0 mg, 0.32 mmol, 1.00 equiv.) in THF (1.00 mL) was added LiOH (23.0 mg, 0.96 mmol, 3.00 equiv.) in H₂O (1.00 mL) at 0° C. The resulting mixture was stirred for 16 h at room temperature. The reaction was quenched with HCl (2M) at 0° C., diluted with water (10 mL), extracted with EA (3×5 mL), washed with NaCl(aq.) and dried over anhydrous Na₂SO₄. After filtration, the filtrate was concentrated under reduced pressure to produce the title compound (90.0 mg, crude) as a yellow solid.

### Example 161D: 3,5-dibromo-N—(4,4-diethyl-7-(trifluoromethyl)-4H-chromeno[4,3-d]thiazol-2-yl)-1-methyl-1H-pyrazole-4-carboxamide

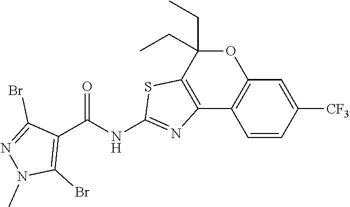 [SMILES: CCC1(CC)Oc2cc(C(F)(F)F)ccc2-c2nc(NC(=O)c3c(Br)nn(C)c3Br)sc21]

To a stirred solution of 3,5-dibromo-1-methyl-1H-pyrazole-4-carboxylic acid (28.0 mg, 0.100 mmol, 1.10 equiv.) and 4,4-diethyl-7-(trifluoromethyl)-4H-chromeno[4,3-d]thiazol-2-amine (30.0 mg, 0.091 mmol, 1.00 equiv.) in ACN (1.00 mL) was added NMI (26.0 mg, 0.320 mmol, 3.50 equiv.) and TCFH (39.0 mg, 0.139 mmol, 1.50 equiv.) in portions at 0° C. The resulting mixture was stirred for 16 h at 60° C. under argon atmosphere. The reaction was diluted with water (20 mL), extracted with EA (3×10 mL), washed with NaCl(aq.) and dried over anhydrous Na₂SO₄. After filtration, the filtrate was concentrated under reduced pressure. The residue was purified by prep-TLC (DCM/PE/EtOAc (7:4:1)) to afford the title compound (28.0 mg, 51.9% yield) as a yellow solid. ESI-MS m/z=592.99 [M+H]⁺; Calculated MW: 591.94.

### Example 161E: 5-bromo-N—(4,4-diethyl-7-(trifluoromethyl)-4H-chromeno[4,3-d]thiazol-2-yl)-3-methoxy-1-methyl-1H-pyrazole-4-carboxamide

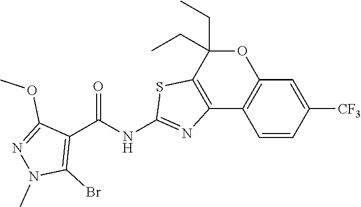 [SMILES: CCC1(CC)Oc2cc(C(F)(F)F)ccc2-c2nc(NC(=O)c3c(OC)nn(C)c3Br)sc21]

To a stirred solution of 3,5-dibromo-N—(4,4-diethyl-7-(trifluoromethyl)-4H-chromeno[4,3-d]thiazol-2-yl)-1-methyl-1H-pyrazole-4-carboxamide (12.0 mg, 0.020 mmol, 1.00 equiv.) in THF (2.00 mL) was added MeONa/MeOH (0.016 mL, 0.080 mmol, 4.00 equiv.) in portions at 0° C. The resulting mixture was stirred for 1 h at 60° C. under argon atmosphere. The reaction was quenched with HCl (2M) at 0° C., diluted with water (10 mL), extracted with EA (3×5 mL), washed with NaCl(aq.) and dried over anhydrous Na₂SO₄. After filtration, the filtrate was concentrated under reduced pressure. The residue was purified by prep-TLC (PE/EtOAc (2:1)) to afford the title compound (5.0 mg, 46% yield) as a yellow solid. ESI-MS m/z=545.08 [M+H]⁺; Calculated MW: 544.04; ¹H NMR (400 MHz, Chloroform-d) δ 9.76 (s, 1H), 7.74 (d, J=7.9 Hz, 1H), 7.17 (d, J=7.9 Hz, 1H), 7.11 (d, J=1.7 Hz, 1H), 4.19 (s, 3H), 3.74 (s, 3H), 2.08-1.96 (m, 2H), 1.88 (dq, J=14.6, 7.4 Hz, 2H), 0.96 (t, J=7.4 Hz, 6H).

### Example 162: N—(4,4-diethyl-7-(trifluoromethyl)-4H-chromeno[4,3-d]thiazol-2-yl)-4,6-dimethoxy-2-methylpyrimidine-5-carboxamide

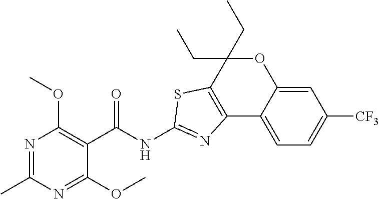 [SMILES: CCC1(CC)Oc2cc(C(F)(F)F)ccc2-c2nc(NC(=O)c3c(OC)nc(C)nc3OC)sc21]

### Example 162A: 4,6-dimethoxy-2-methylpyrimidine-5-carboxylic acid

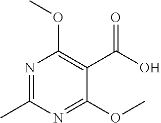

To a stirred solution of 4,6-dichloro-2-methylpyrimidine-5-carboxylic acid (100 mg, 0.48 mmol, 1.0 equiv.) in MeOH (3 mL) was added a solution of KOH (80.6 mg, 1.44 mmol, 3.0 equiv.) in MeOH (1 mL) dropwise at 0° C. After being stirred overnight at 70° C., the pH of the mixture was adjusted to 4-5 by 4M HCl. Then the mixture was filtered and wash by EtOH (5 mL×3). The organic phase was concentrated under vacuum to afford the title compound (105 mg, crude) as a yellow solid. ¹H NMR (400 MHz, DMSO-d₆) δ 3.92 (s, 3H), 3.87 (s, 3H), 2.49 (s, 3H).

### Example 162B: N—(4,4-diethyl-7-(trifluoromethyl)-4H-chromeno[4,3-d]thiazol-2-yl)-4,6-dimethoxy-2-methylpyrimidine-5-carboxamide

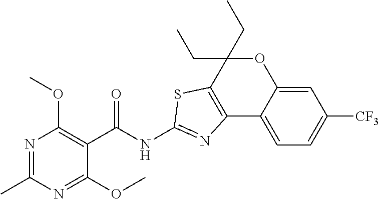 [SMILES: CCC1(CC)Oc2cc(C(F)(F)F)ccc2-c2nc(NC(=O)c3c(OC)nc(C)nc3OC)sc21]

To a stirred solution of 4,4-diethyl-7-(trifluoromethyl)-4H-chromeno[4,3-d]thiazol-2-amine (30 mg, 0.091 mmol, 1.0 equiv.) in MeCN (1 mL) was added 4,6-dimethoxy-2-methylpyrimidine-5-carboxylic acid (26.9 mg, 0.137 mmol, 1.5 equiv.), TCFH (38.4 mg, 0.137 mmol, 1.5 equiv.) and NMI (22.4 mg, 0.273 mmol, 3.5 equiv.) at 0° C. The reaction mixture was stirred for 15 min at room temperature, then overnight at 50° C. After concentration, the resulting residue was diluted with water, extracted with ethyl acetate (3×10 mL). The combined organic layers were washed with brine, dried over anhydrous Na₂SO₄. The resulting mixture was concentrated under vacuum. The crude product was purified by Prep TLC (DCM) to afford the title compound (2.2 mg, 14%) as a white solid. ESI-MS m/z=509.17 [M+H]⁺; Calculated MW: 508.14; ¹H NMR (400 MHz, Chloroform-d) δ 11.40 (s, 1H), 7.71 (d, J=7.8 Hz, 1H), 7.13 (d, J=10.4 Hz, 2H), 4.06 (s, 6H), 2.48 (s, 3H), 2.02 (dq, J=14.7, 7.4 Hz, 2H), 1.89 (dq, J=14.7, 7.3 Hz, 2H), 0.98 (t, J=7.4 Hz, 6H).

### Example 163: methyl 2-(2′-(4,6-dimethoxypyrimidine-5-carboxamido)-7′-(trifluoromethyl)spiro[azetidine-3,4′-chromeno[4,3-d]thiazol]-1-yl) acetate

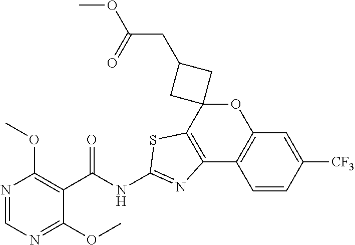 [SMILES: COC(=O)CC1CC2(C1)Oc1cc(C(F)(F)F)ccc1-c1nc(NC(=O)c3c(OC)ncnc3OC)sc12]

To a mixture of 4,6-dimethoxy-N—(7′-(trifluoromethyl)spiro[azetidine-3,4′-chromeno[4,3-d]thiazol]-2′-yl)pyrimidine-5-carboxamide (50 mg, 104.29 μmol, 1 equiv.) and K₂CO₃ (21.6 mg, 156.43 umol, 1.5 equiv.) in CH₃CN (1 mL) at 0° C. was added a solution of methyl 2-bromoacetate (16.0 mg, 104.29 μmol, 1 equiv.) in CH₃CN (1 mL) at the same temperature. The reaction mixture was stirred at 0 to rt for 1 hour. The mixture was quenched with ice-water (5 mL). The aqueous layer was extracted with EA (20 mL×2) for twice. The combined organic layers were washed with brine (20 mL×2), dried over Na₂SO₄, filtrated, and concentrated under reduce pressure. The residue was purified by prep-TLC (DCM:MeOH=20:1) to produce methyl 2-(2′-(4,6-dimethoxypyrimidine-5-carboxamido)-7′-(trifluoromethyl)spiro[azetidine-3,4′-chromeno[4,3-d]thiazol]-1-yl)acetate (10 mg, 17%) as white solid. ESI-MS m/z=552.17 [M+H]⁺; Calculated MW: 551.49. ¹H NMR (400 MHz, DMSO-d₆) δ 12.92 (s, 1H), 8.60 (s, 1H), 7.69 (d, J=7.9 Hz, 1H), 7.35 (dd, J=8.1, 1.7 Hz, 1H), 7.25 (d, J=1.7 Hz, 1H), 3.93 (s, 6H), 3.77 (d, J=7.5 Hz, 2H), 3.70-3.65 (m, 5H), 3.45 (s, 2H).

### Example 164: N—(1-(2-(dimethylamino)ethyl)-7′-(trifluoromethyl)spiro[azetidine-3,4′-chromeno[4,3-d]thiazol]-2′-yl)-4,6-dimethoxypyrimidine-5-carboxamide

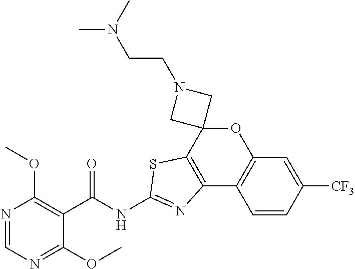 [SMILES: COc1ncnc(OC)c1C(=O)Nc1nc2c(s1)C1(CN(CCN(C)C)C1)Oc1cc(C(F)(F)F)ccc1-2]

### Example 164A: tert-butyl(2-(2′-(4,6-dimethoxypyrimidine-5-carboxamido)-7′-(trifluoromethyl)spiro[azetidine-3,4′-chromeno[4,3-d]thiazol]-1-yl)ethyl)(methyl)carbamate

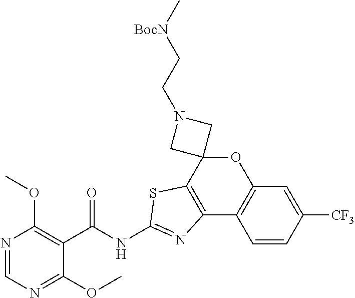 [SMILES: COc1ncnc(OC)c1C(=O)Nc1nc2c(s1)C1(CN(CCN(C)C(=O)OC(C)(C)C)C1)Oc1cc(C(F)(F)F)ccc1-2]

4,6-dimethoxy-N—(7′-(trifluoromethyl)spiro[azetidine-3,4′-chromeno[4,3-d]thiazol]-2′-yl)pyrimidine-5-carboxamide (260 mg, 542.31 μmol, 1 equiv.) was added to a solution of tert-butyl methyl(2-oxoethyl)carbamate in DCE (5.2 mL). AcOH (62.0 μl, 65.1 mg, 1.08 mmol, 2 equiv.) was diluted with DCE (0.5 mL) and added to the mixture at rt. The reaction mixture was stirred at rt for 15 minutes. NaBH(OAc)₃ (344.8 mg, 1.63 mmol, 3 equiv.) was added to the mixture at 0° C. The reaction mixture was stirred at rt for 1 hour and quenched with ice-water (5 mL) and sat. NH₄Cl (5 mL). The aqueous layer was extracted with EA (20 mL×2). The combined organic layers were washed with brine (20 mL×2), dried over Na₂SO₄, filtrated, and concentrated under reduce pressure. The residue was purified by prep-TLC (DCM:MeOH=50:1) to produce tert-butyl(2-(2′-(4,6-dimethoxypyrimidine-5-carboxamido)-7′-(trifluoromethyl)spiro[azetidine-3,4′-chromeno[4,3-d]thiazol]-1-yl)ethyl)(methyl)carbamate (158 mg, 45.8%) as yellow solid. ¹H NMR (400 MHz, DMSO-d₆) δ 12.88 (s, 1H), 8.59 (s, 1H), 7.69 (d, J=7.9 Hz, 1H), 7.39-7.32 (m, 1H), 7.24 (s, 1H), 3.93 (s, 6H), 3.73-3.65 (m, 2H), 3.42 (s, 2H), 3.16 (t, J=6.4 Hz, 2H), 2.81 (d, J=10.4 Hz, 3H), 2.62 (s, 2H), 1.36 (s, 9H).

### Example 164B: 4,6-dimethoxy-N—(1-(2-(methylamino)ethyl)-7′-(trifluoromethyl)spiro[azetidine-3,4′-chromeno[4,3-d]thiazol]-2′-yl)pyrimidine-5-carboxamide

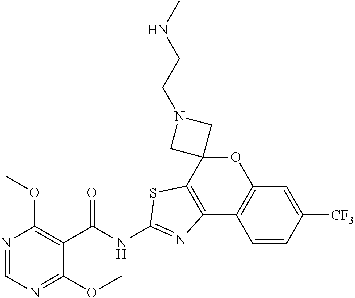 [SMILES: CNCCN1CC2(C1)Oc1cc(C(F)(F)F)ccc1-c1nc(NC(=O)c3c(OC)ncnc3OC)sc12]

Tert-butyl(2-(2′-(4,6-dimethoxypyrimidine-5-carboxamido)-7′-(trifluoromethyl)spiro[azetidine-3,4′-chromeno[4,3-d]thiazol]-1-yl)ethyl)(methyl)carbamate (158 mg, 248.18 μmol, 1 equiv.) was added to a flame-dried flask. DCM (3 mL) was added and the mixture was cooled to 0° C. TFA (1.5 mL) was added to the mixture at 0° C. The reaction mixture was stirred at rt for 1 hour. The mixture was concentrated under reduce pressure and quenched with sat. NaHCO₃. The aqueous layer was extracted with EA (20 mL×2) for twice. The combined organic layers were washed with brine (20 mL×2), dried over Na₂SO₄, filtrated, and concentrated under reduce pressure to produce 4,6-dimethoxy-N—(1-(2-(methylamino)ethyl)-7′-(trifluoromethyl)spiro[azetidine-3,4′-chromeno[4,3-d]thiazol]-2′-yl)pyrimidine-5-carboxamide (100 mg, 75.1%) as yellow solid. ¹H NMR (400 MHz, DMSO-d₆) δ 8.59 (s, 1H), 7.70 (d, J=7.9 Hz, 1H), 7.35 (dd, J=9.4, 7.4 Hz, 1H), 7.24 (d, J=5.7 Hz, 1H), 3.92 (s, 6H), 3.74 (s, 4H), 2.77 (s, 4H), 2.50 (s, 3H).

### Example 164C: N—(1-(2-(dimethylamino)ethyl)-7′-(trifluoromethyl)spiro[azetidine-3,4′-chromeno[4,3-d]thiazol]-2′-yl)-4,6-dimethoxypyrimidine-5-carboxamide

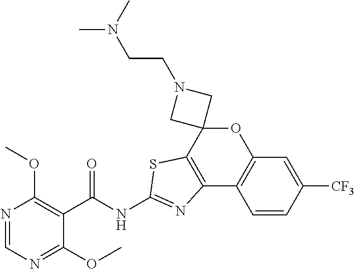 [SMILES: COc1ncnc(OC)c1C(=O)Nc1nc2c(s1)C1(CN(CCN(C)C)C1)Oc1cc(C(F)(F)F)ccc1-2]

4,6-dimethoxy-N—(1-(2-(methylamino)ethyl)-7′-(trifluoromethyl)spiro[azetidine-3,4′-chromeno[4,3-d]thiazol]-2′-yl)pyrimidine-5-carboxamide (50 mg, 93.19 μmol, 1 equiv.) and DCE (1 mL) were added to a flame-dried flask. 36% aqueous CH₂O (36.0 μl, 38.9 mg, 465.96 μmol, 5 equiv.) was added to the mixture at rt. NaBH(OAc)₃ (29.6 mg, 139.79 μmol, 1.5 equiv.) was added to the mixture at 0° C. The reaction mixture was stirred at rt for 16 hours. The mixture was quenched with ice-water (5 mL) and sat. NH₄Cl (5 mL). The aqueous layer was extracted with EA (20 mL×2). The combined organic layers were washed with brine (20 mL×2), dried over Na₂SO₄, filtrated, and concentrated under reduce pressure. The residue was purified by prep-TLC (PE:EA=2:1) to produce N—(1-(2-(dimethylamino)ethyl)-7′-(trifluoromethyl)spiro[azetidine-3,4′-chromeno[4,3-d]thiazol]-2′-yl)-4,6-dimethoxypyrimidine-5-carboxamide (15 mg, 29%) as white solid. ESI-MS m/z=551.20 [M+H]⁺; Calculated MW: 550.55. ¹H NMR (400 MHz, DMSO-d₆) δ 8.59 (s, 1H), 7.68 (d, J=7.9 Hz, 1H), 7.34 (dd, J=8.2, 1.7 Hz, 1H), 7.25 (d, J=1.7 Hz, 1H), 3.92 (s, 6H), 3.71-3.64 (m, 2H), 3.43 (d, J=7.5 Hz, 2H), 2.64 (t, J=6.8 Hz, 2H), 2.36 (s, 2H), 2.22 (s, 6H).

### Example 165:2-(2′-(4,6-dimethoxypyrimidine-5-carboxamido)-7′-(trifluoromethyl)spiro[azetidine-3,4′-chromeno[4,3-d]thiazol]-1-yl) acetic acid

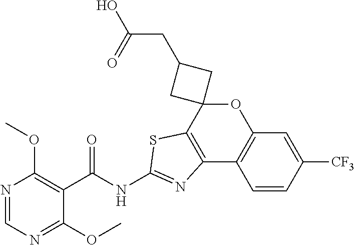 [SMILES: COc1ncnc(OC)c1C(=O)Nc1nc2c(s1)C1(CC(CC(=O)O)C1)Oc1cc(C(F)(F)F)ccc1-2]

To a solution of the methyl 2-(2′-(4,6-dimethoxypyrimidine-5-carboxamido)-7′-(trifluoromethyl)spiro[azetidine-3,4′-chromeno[4,3-d]thiazol]-1-yl)acetate (50 mg, 90.66 μmol, 1 equiv.) in THF (1 mL) was added LiOH (6.5 mg, 271.99 μmol, 3 equiv.) in H₂O (0.5 mL) at 0° C. The mixture was stirred at room temperature for 16 h. The reaction was neutralized with aq. HCl (1M) to pH 7, and extracted with EA (3×20 mL). The combined organic layers were washed with brine (20 mL×2), dried over Na₂SO₄, filtered, and concentrated under reduce pressure. The crude product was purified by pulping from DCM (5 mL) to afford 2-(2′-(4,6-dimethoxypyrimidine-5-carboxamido)-7′-(trifluoromethyl)spiro[azetidine-3,4′-chromeno[4,3-d]thiazol]-1-yl) acetic acid (17.9 mg, 36.7% yield) as white solid. ESI-MS m/z=538.17 [M+H]⁺; Calculated MW: 537.47. ¹H NMR (400 MHz, DMSO-d₆) δ 12.91 (s, 1H), 8.59 (s, 1H), 7.69 (d, J=7.9 Hz, 1H), 7.35 (dd, J=8.2, 1.8 Hz, 1H), 7.25 (d, J=1.8 Hz, 1H), 3.93 (s, 6H), 3.78 (d, J=7.4 Hz, 2H), 3.62 (d, J=7.6 Hz, 2H), 3.36 (s, 2H).

### Example 166: N—(1-(2-hydroxyethyl)-7′-(trifluoromethyl)spiro[azetidine-3,4′-chromeno[4,3-d]thiazol]-2′-yl)-4,6-dimethoxypyrimidine-5-carboxamide

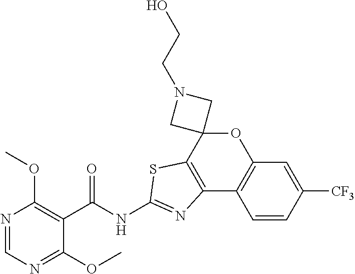 [SMILES: COc1ncnc(OC)c1C(=O)Nc1nc2c(s1)C1(CN(CCO)C1)Oc1cc(C(F)(F)F)ccc1-2]

### Example 166A: N—(1-(2-((tert-butyldimethylsilyl)oxy) ethyl)-7′-(trifluoromethyl)spiro[azetidine-3,4′-chromeno[4,3-d]thiazol]-2′-yl)-4,6-dimethoxypyrimidine-5-carboxamide

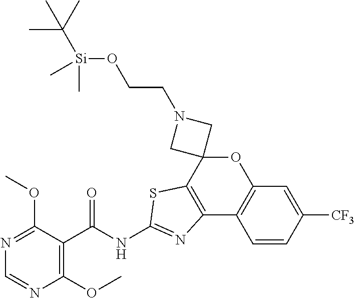 [SMILES: COc1ncnc(OC)c1C(=O)Nc1nc2c(s1)C1(CN(CCO[Si](C)(C)C(C)(C)C)C1)Oc1cc(C(F)(F)F)ccc1-2]

4,6-dimethoxy-N—(7′-(trifluoromethyl)spiro[azetidine-3,4′-chromeno[4,3-d]thiazol]-2′-yl)pyrimidine-5-carboxamide (100 mg, 208.58 μmol, 1 equiv.) and DCE (2 mL) were added to a flame-dried over flask. 2-((tert-butyldimethylsilyl)oxy) acetaldehyde (0.199 mL, 1.04 mmol, 5 equiv.) was diluted with DCE (0.4 mL) and added to the mixture at rt. AcOH (23.9 μL, 417.16 μmol, 2 equiv.) was diluted with DCE (0.4 mL) and added to the mixture at rt. The reaction mixture was stirred at rt for 15 minutes. NaBH(OAc)₃ (132.6 mg, 625.74 μmol, 3 equiv.) was added to the mixture at 0° C. The reaction mixture was stirred at rt for 1 hour and quenched with ice-water (10 mL) and sat. NH₄Cl (10 mL). The aqueous layer was extracted with EA (20×3 mL). The combined organic layers were washed with brine (20 mL×2), dried over Na₂SO₄, filtered, and concentrated under reduce pressure. The residue was purified by prep-TLC (PE:EA=2:1) to produce N—(1-(2-((tert-butyldimethylsilyl)oxy) ethyl)-7′-(trifluoromethyl)spiro[azetidine-3,4′-chromeno[4,3-d]thiazol]-2′-yl)-4,6-dimethoxypyrimidine-5-carboxamide (107 mg, 80.5%) as white solid. ESI-MS m/z=638.42 [M+H]⁺; Calculated MW: 637.75. ¹H NMR (400 MHz, DMSO-d₆) δ 12.90 (s, 1H), 8.59 (s, 1H), 7.68 (d, J=7.9 Hz, 1H), 7.37-7.30 (m, 1H), 7.24 (d, J=1.7 Hz, 1H), 3.93 (s, 6H), 3.74-3.66 (m, 2H), 3.62 (t, J=5.5 Hz, 2H), 3.50-3.44 (m, 2H), 2.62 (t, J=5.4 Hz, 2H), 0.83 (d, J=5.2 Hz, 9H), 0.04-0.02 (m, 6H).

### Example 166B: N—(1-(2-hydroxyethyl)-7′-(trifluoromethyl)spiro[azetidine-3,4′-chromeno[4,3-d]thiazol]-2′-yl)-4,6-dimethoxypyrimidine-5-carboxamide

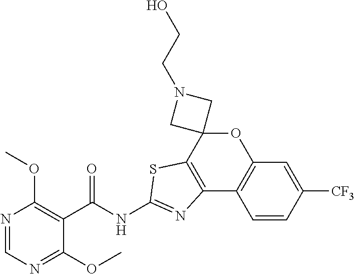 [SMILES: COc1ncnc(OC)c1C(=O)Nc1nc2c(s1)C1(CN(CCO)C1)Oc1cc(C(F)(F)F)ccc1-2]

To a solution of the N—(1-(2-((tert-butyldimethylsilyl)oxy) ethyl)-7′-(trifluoromethyl)spiro[azetidine-3,4′-chromeno[4,3-d]thiazol]-2′-yl)-4,6-dimethoxypyrimidine-5-carboxamide (50 mg, 70.48 μmol, 1 equiv.) in THF (1 mL) was added tetrabutylammonium fluoride (0.16 mL, 156.8 μmol, 2 equiv., 1M) at 0° C. The mixture was stirred at rt for 3h. Water (20 mL) was added to the reaction mixture and the aqueous phase was extracted with EA (3× 20 mL). The combined organic layers were washed with brine (2×20 mL), dried over Na₂SO₄, filtered, and concentrated under reduce pressure. The residue was purified by prep-TLC (PE:EA=1:2) to produce N—(1-(2-hydroxyethyl)-7′-(trifluoromethyl)spiro[azetidine-3,4′-chromeno[4,3-d]thiazol]-2′-yl)-4,6-dimethoxypyrimidine-5-carboxamide (16.4 mg, 40%) as white solid. ESI-MS m/z=524.23 [M+H]⁺; Calculated MW: 523.49. ¹H NMR (400 MHz, DMSO-d₆) δ 12.90 (s, 1H), 8.59 (s, 1H), 7.68 (d, J=7.9 Hz, 1H), 7.35-7.31 (m, 1H), 7.25 (d, J=1.7 Hz, 1H), 4.50 (t, J=5.3 Hz, 1H), 3.93 (s, 6H), 3.72-3.64 (m, 2H), 3.46 (d, J=8.1 Hz, 2H), 3.41 (t, J=5.7 Hz, 2H), 2.58 (t, J=5.9 Hz, 2H).

### Example 167: 4,6-dimethoxy-N—(1-(2-methyl-4-oxopentan-2-yl)-7′-(trifluoromethyl)spiro[azetidine-3,4′-chromeno[4,3-d]thiazol]-2′-yl)pyrimidine-5-carboxamide

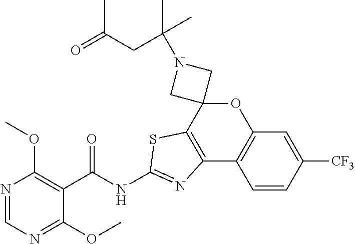 [SMILES: COc1ncnc(OC)c1C(=O)Nc1nc2c(s1)C1(CN(C(C)(C)CC(C)=O)C1)Oc1cc(C(F)(F)F)ccc1-2]

4,6-dimethoxy-N—(7′-(trifluoromethyl)spiro[azetidine-3,4′-chromeno[4,3-d]thiazol]-2′-yl)pyrimidine-5-carboxamide (712 mg, 1.49 mmol, 1 equiv.) and DCE (14 mL) were added to a flame-dried flask Acetone (0.547 mL, 431.3 mg, 7.43 mmol, 5 equiv.) and AcOH (0.170 mL, 178.36 mg, 2.97 mmol, 2 equiv.) were added to the mixture at rt. The reaction mixture was stirred at rt for 15 minutes. NaBH(OAc)₃ (944.2 mg, 4.64 mmol, 3 equiv.) was added to the mixture at 0° C. The reaction mixture was stirred at rt for 1 hour and quenched with ice-water (20 mL) and sat. NH₄Cl (20 mL). The aqueous layer was extracted with EA (100 mL×2). The combined organic layers were washed with brine (200 mL×2), dried over Na₂SO₄, filtered, and concentrated under reduce pressure. The residue was purified by silica gel chromatography column (PE:EA=10:1 to 3:1) to produce 4,6-dimethoxy-N—(1-(2-methyl-4-oxopentan-2-yl)-7′-(trifluoromethyl)spiro[azetidine-3,4′-chromeno[4,3-d]thiazol]-2′-yl)pyrimidine-5-carboxamide (197 mg, 23%) as white solid. ESI-MS m/z=578.35 [M+H]⁺; Calculated MW: 577.57.

¹H NMR (400 MHz, DMSO-d₆) δ 12.89 (s, 1H), 8.60 (s, 1H), 7.69 (d, J=7.9 Hz, 1H), 7.34 (d, J=8.1 Hz, 1H), 7.27 (d, J=1.7 Hz, 1H), 3.93 (s, 6H), 3.60 (d, J=7.3 Hz, 2H), 3.52-3.44 (m, 2H), 2.41 (s, 2H), 2.16 (s, 3H), 1.02 (s, 6H).

### Example 168: tert-butyl 2′-(4,6-dimethoxypyrimidine-5-carboxamido)-7′-(trifluoromethyl)spiro[azetidine-3,4′-chromeno[4,3-d][1,3]selenazole]-1-carboxylate

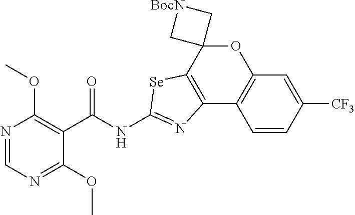 [SMILES: COc1ncnc(OC)c1C(=O)Nc1nc2c([se]1)C1(CN(C(=O)OC(C)(C)C)C1)Oc1cc(C(F)(F)F)ccc1-2]

### Example 168A: tert-butyl 2′-amino-7′-(trifluoromethyl)spiro[azetidine-3,4′-chromeno[4,3-d][1,3]selenazole]-1-carboxylate

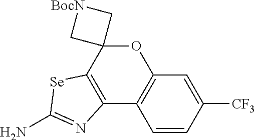 [SMILES: CC(C)(C)OC(=O)N1CC2(C1)Oc1cc(C(F)(F)F)ccc1-c1nc(N)[se]c12]

Into a flask was added 3′-chloro-4′-oxo-7′-(trifluoromethyl)spiro[azetidine-3,2′-chromane]-1-carboxylate (500 mg, 1.28 mmol, 1 equiv.), selenourea (486.8 mg, 3.96 mmol, 3.1 equiv.) and MeOH (10 mL). After stirring at 100° C. for 16 hours the mixture was concentrated under reduce pressure and diluted with EA (30 mL). The organic layer was washed with brine (30 mL×2), dried over Na₂SO₄, filtrated, and concentrated under reduce pressure. The residue was purified by prep-TLC (PE:EA=2:1) to obtain tert-butyl 2′-amino-7′-(trifluoromethyl)spiro[azetidine-3,4′-chromeno[4,3-d][1,3]selenazole]-1-carboxylate (260 mg, 44.3%) as a yellow solid. ¹H NMR (400 MHz, Chloroform-d) δ 7.67 (dd, J=8.0, 1.0 Hz, 1H), 7.22 (ddd, J=7.9, 1.8, 0.8 Hz, 1H), 7.16 (d, J=1.7 Hz, 1H), 5.44 (br, 2H), 4.40 (dd, J=9.7, 1.1 Hz, 2H), 4.20-4.12 (m, 2H), 1.46 (s, 9H).

### Example 168B: tert-butyl 2′-(4,6-dimethoxypyrimidine-5-carboxamido)-7′-(trifluoromethyl)spiro[azetidine-3,4′-chromeno[4,3-d][1,3]selenazole]-1-carboxylate

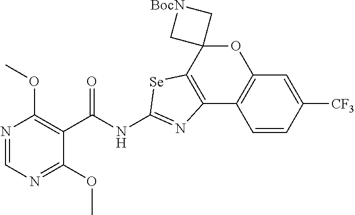 [SMILES: COc1ncnc(OC)c1C(=O)Nc1nc2c([se]1)C1(CN(C(=O)OC(C)(C)C)C1)Oc1cc(C(F)(F)F)ccc1-2]

Into a flask was added tert-butyl 2′-amino-7′-(trifluoromethyl)spiro[azetidine-3,4′-chromeno[4,3-d][1,3]selenazole]-1-carboxylate (47 mg, 0.102 mmol, 1 equiv.), 4,6-dimethoxypyrimidine-5-carboxylic acid (28.2 mg, 0.153 mmol, 1.5 equiv.) and ACN (1 mL). After cooled to 0° C., NMI (29.3 mg, 27.94 mml, 0.357 mmol, 3.5 equiv.) and TCFH (43 mg in 0.5 mL ACN, 0.153 mmol, 1.5 equiv.) were added. The reaction was stirred at rt for 3 hours and then stirred at 60° C. for 16 hours. After cooled to rt, the mixture was concentrated under reduce pressure then quenched with ice-water (10 mL). The aqueous was extracted with EtOAc (10 mL×3) and the combined organic layers were washed with brine (20 mL×2), dried over Na₂SO₄, filtrated, and concentrated under reduce pressure. The crude product was purified by prep-TLC (PE:EA=1:1) to afford tert-butyl 2′-(4,6-dimethoxypyrimidine-5-carboxamido)-7′-(trifluoromethyl)spiro[azetidine-3,4′-chromeno[4,3-d][1,3]selenazole]-1-carboxylate (40 mg, 62.5%) as a white solid. ESI-MS m/z=628.09 [M+H]⁺; Calculated MW: 626.46. ¹H NMR (400 MHz, Chloroform-d) δ 11.07 (s, 1H), 8.45 (s, 1H), 7.80 (d, J=7.9 Hz, 1H), 7.22 (t, J=1.8 Hz, 2H), 4.46 (dd, J=9.5, 1.0 Hz, 2H), 4.24-4.16 (m, 2H), 4.11 (s, 6H), 1.47 (s, 9H).

### Example 169: Provided technologies can inhibit TRPV3 activation

Various technologies can be utilized to assess TRPV3 activation in accordance with the present disclosure. For example, in some embodiments, compounds are assessed in in vitro patch clamp experiments. Certain useful procedures are described herein as examples.

In some embodiments, whole-cell patch-clamp recordings were used to detect the current through hTRPV3 channels in the HEK293T cell line transfected with hTRPV3 cDNA and evaluate the exact blocking effect of compounds on hTRPV3. In some embodiments, a perfusion system was adopted to control the extracellular solution, including the addition of the agonist and different antagonists. The current of hTRPV3 is usually induced by addition of 300 μM 2-APB.

HEK293T cells were cultured in a Dulbecco's modified Eagle's medium containing 10% fetal bovine serum (Thermo Scientific) and 1% penicillin/streptomycin (Thermo Scientific) at 37° C. with 5% CO₂. The commercially available plasmid with the full-length cDNA of hTRPV3 (791 aa) (NM_001258205.2) was purchased from OriGene Technologies. The transfection of hTRPV3 plasmids was conducted using Lipofectamine™ 3000 following the manufacturer's instructions (Invitrogen).

Whole-cell patch-clamp experiments were performed 18 h post-transfection, using a MultiClamp 700B amplifier driven by pClamp 11 software (Molecular Devices). In order to achieve good single-cell physical separation, HEK293T cells with hTRPV3 expression were digested and then re-plated at low density on glass coverslips. Electrodes were pulled from borosilicate glass with a P-97 micropipette puller (Sutter Instruments) and those with resistances of 3-5 MΩ are used for experiments. In some embodiments, an Ag—AgCl wire was used as a reference electrode and signals are filtered using a 2.9 kHz low-pass Bessel filter. Membrane potential was held at 0 mV. For recording of agonist-induced activation and inhibition by different test compounds, currents were elicited by a protocol consisting of a 400-ms step to +80 mV, followed by a 400-ms step to −80 mV at 2-s intervals. both pipette and bath solutions contained 130 mM NaCl, 0.3 mM EDTA, and 3 mM HEPES (buffered to pH 7.4 with NaOH). All recordings were made at room temperature.

All patch-clamp recordings were analyzed using the Clampfit software and GraphPad Prism 6.0 software. Specifically, data are analyzed by calculating the degree of current amplitude after the test compound addition compared with the unblocked agonist-induced current amplitude. The resulting blocking percentage at each concentration was used to make a concentration-response analysis and fitted to the Hill equation: blocking percentage=minimum blocking percentage+ (maximum blocking percentage-minimum blocking percentage)/(1+10\hat{ }(Hill slope* (logIC₅₀))). The minimum blocking percentage is 0% and maximum blocking percentage is 100%, while the Hill slope and the IC₅₀ were determined as the curve fitting routine. Compounds of interest were tested against hTRPV3 at concentrations up to 10 μM, and certain data was used to estimate IC₅₀ as described herein.

Table 1 provides data obtained in this assay for certain provided compounds. A: IC₅₀ less than 0.1 μM; B: IC₅₀ between 0.1 μM (inclusive) and 1.0 μM; C″ IC₅₀ between 1.0 μM (inclusive) and 10.0 μM; and D: IC₅₀>=10 μM. ND: data not determined.

TABLE 1
Certain results for certain compounds.

| Example # | IC₅₀ (μM) |
|---|---|
| 1 | B |
| 2 | D |
| 3 | C |
| 4 | D |
| 5 | D |
| 6 | D |
| 7 | C |
| 8 | B |
| 9 | D |
| 10 | D |
| 11 | D |
| 12 | B |
| 13 | A |
| 14 | C |
| 15 | D |
| 16 | D |
| 17 | D |
| 18 | C |
| 19 | B |
| 20 | B |
| 21 | B |
| 22 | D |
| 23 | B |
| 24 | A |
| 25 | A |
| 26 | B |
| 27 | A |
| 28 | B |
| 29 | B |
| 30 | B |
| 31 | B |
| 32 | A |
| 33 | B |
| 34 | B |
| 35 | B |
| 36 | B |
| 37 | B |
| 38 | B |
| 39 | B |
| 40 | B |
| 41 | B |
| 42 | B |
| 43 | B |
| 44 | A |
| 45 | D |
| 46 | C |
| 47 | B |
| 48 | D |
| 49 | C |
| 50 | B |
| 51 | D |
| 52 | C |
| 53 | D |
| 54 | B |
| 55 | B |
| 56 | D |
| 57 | B |
| 58 | C |
| 59 | B |
| 60 | B |
| 61 | C |
| 62 | C |
| 63 | A |
| 64 | B |
| 65 | B |
| 66 | B |
| 67 | D |
| 68 | C |
| 69 | B |
| 70 | B |
| 71 | D |
| 72 | C |
| 73 | C |
| 74 | B |
| 75 | B |
| 76 | C |
| 77 | B |
| 78 | B |
| 79 | D |
| 80 | D |
| 81 | B |
| 82 | B |
| 83 | D |
| 84 | C |
| 85 | C |
| 86 | C |
| 87 | B |
| 88 | B |
| 89 | D |
| 90 | C |
| 91 | D |
| 92 | B |
| 93 | C |
| 94 | D |
| 95 | C |
| 96 | C |
| 97 | B |
| 98 | B |
| 99 | D |
| 100 | A |
| 101 | D |
| 102 | D |
| 103 | C |
| 104 | B |
| 105 | B |
| 106 | B |
| 107 | C |
| 108 | B |
| 109 | B |
| 110 | C |
| 111 | C |
| 112 | B |
| 113 | C |
| 114 | C |
| 115 | B |
| 116 | C |
| 117 | C |
| 118 | B |
| 119 | C |
| 120 | C |
| 121 | D |
| 122 | D |
| 123 | A |
| 124 | B |
| 125 | C |
| 126 | B |
| 127 | B |
| 128 | B |
| 129 | B |
| 130 | C |
| 131 | B |
| 132 | B |
| 133 | A |
| 134 | B |
| 135 | A |
| 136 | A |
| 137 | D |
| 138 | B |
| 139 | D |
| 140 | C |
| 141 | C |
| 142 | D |
| 143 | D |
| 144 | D |
| 145 | B |
| 146 | A |
| 147 | C |
| 148 | B |
| 149 | A |
| 150 | A |
| 151 | C |
| 152 | B |
| 153 | A |
| 154 | B |
| 155 | A |
| 156 | C |
| 157 | A |
| 158 | D |
| 159 | A |
| 160 | B |
| 161 | D |
| 162 | C |
| 163 | A |
| 164 | C |
| 165 | D |
| 166 | B |
| 167 | B |
| 168 | B |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. A compound, wherein the compound has the structure of formula I: 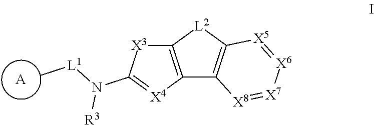
or a salt thereof, wherein:
Ring A is an optionally substituted 5-6 membered aromatic ring having 0-4 heteroatoms, or is 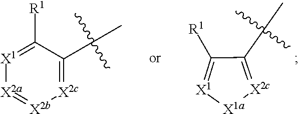
each of X¹, X^{2a}, X^{2b}, and X^{2c} is independently —N═, —C(R²)═ or optionally substituted —CH═;
X^{1a} is —O—, —S—, —N(R′)—, —C(R′)₂—, or optionally substituted —CH₂— or —NH—;
X³ is —O—, —S—, —Se—, —N(R′)— or optionally substituted —NH—;
each of X⁴, X⁵, X⁶, X⁷ and X⁸ is independently —N═, —C(R⁶)═ or optionally substituted —CH═;
each of R¹, R², R^{s} and R⁶ is independently halogen, —CN, —NO₂, -L-R′, —OR′, —N(R′)₂, —N(R′)C(O)OR′, —C(O)R′, —C(O)OR′, —C(O)N(R′)₂, or —OC(O)N(R′)₂;
L¹ is a covalent bond, —C(O)—, —C(S)—, —S(O)₂—, —N(R′)C(O)—, —N(R′)S(O)₂—, —N(R′)C(S)— or 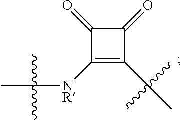
each of R³, R⁴ and R⁵ is independently R′;
each of L and L² is independently optionally substituted C₁₋₃ alkylene, wherein one or more methylene units of the alkylene are optionally and independently replaced with —O—, —S—, —C(R⁴)(R⁵)—, —N(R⁴)—, optionally substituted —CH═N—, optionally substituted —CH═CH— or -Cy-; 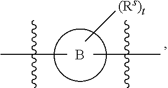
each -Cy- is independently wherein Ring B is an optionally substituted saturated or partially saturated 3-10 membered ring having 0-4 heteroatoms;
t is 0, 1, 2, 3 or 4;
each R′ is independently R, —OR, —C(O)R, —C(O)OR, or —S(O)₂R;
each R is independently hydrogen or an optionally substituted group selected from C₁-C₁₀ aliphatic, C₁-C₁₀ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₁₀ aliphatic, and 5-10 membered heteroaryl having 1-6 heteroatoms-C₁-C₁₀ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

2. The compound of claim 1, wherein Ring A is optionally substituted 5-6 membered heteroaryl ring having 1, 2, 3, or 4 heteroatoms.

3. The compound of claim 1 or 2, wherein Ring A is optionally substituted

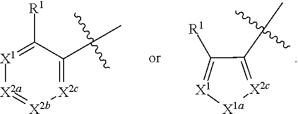

4. The compound of any one of claims 1-3, wherein X¹ is —N═ or —C(R²)═.

5. The compound of any one of claims 1-4, wherein X^{2a} is —N═ or —C(R²)—.

6. The compound of any one of claims 1-5, wherein X^{2b} is —N═ or —C(R²)═.

7. The compound of any one of claims 1-6, wherein X^{2c} is —N═ or —C(R²)═.

8. The compound of any one of claims 1-7, wherein X^{1a} is —N(R′)—, —O—, —S—, or —C(R′)₂—.

9. The compound of any one of claims 1-8, wherein Ring A is

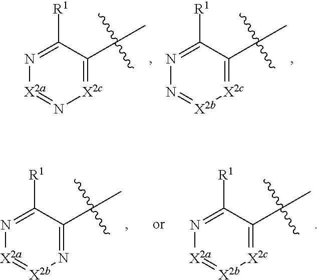

10. The compound of any one of claims 1-9, wherein Ring A is

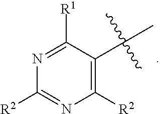

11. The compound of any one of claims 1-8, wherein Ring A is

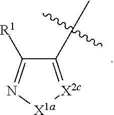

12. The compound of claim 11, wherein Ring A is

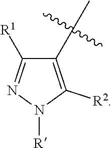

13. The compound of any one of claims 1-12, wherein X³ is —S—, —Se—, —O—, or —N(R′)—.

14. The compound of any one of claims 1-13, wherein X⁴ is —N═ or —C(R⁶)═.

15. The compound of any one of claims 1-14, wherein X⁵ is —N═ or —C(R⁶)—.

16. The compound of any one of claims 1-15, wherein X⁶ is —N═ or —C(R⁶)═.

17. The compound of any one of claims 1-16, wherein X⁶ is —C(OR′)═ wherein R′ is taken together with another R′ group on another atom and their intervening atoms to form an optionally substituted 3-10 membered ring having, in addition to the intervening atoms, 0-4 heteroatoms.

18. The compound of any one of claims 1-17, wherein X⁷ is —N═ or —C(R⁶)═.

19. The compound of any one of claims 1-18, wherein X⁷ is —C(OR′)═ wherein R′ is taken together with another R′ group on another atom and their intervening atoms to form an optionally substituted 3-10 membered ring having, in addition to the intervening atoms, 0-4 heteroatoms.

20. The compound of any one of claims 1-19, wherein X⁸ is —N═, or —C(R⁶)═.

21. The compound of any one of claims 1-20, wherein X⁸ is —C(OR′)═ wherein R′ is taken together with another R′ group on another atom and their intervening atoms to form an optionally substituted 3-10 membered ring having, in addition to the intervening atoms, 0-4 heteroatoms.

22. The compound of any one of claims 1-21, wherein R¹ is —OR′, optionally substituted C₁-C₁₀ aliphatic, or halogen.

23. The compound of any one of claims 1-22, wherein L¹ is covalent bond, —C(O)—, —C(S)—, —S(O)₂—, optionally substituted —CH₂—, —N(R′)C(O)—, —N(R′)S(O)₂—, —N(R′)C(S)—, or

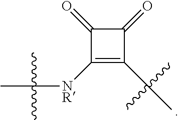

24. The compound of any one of claims 1-23, wherein R³ is H or optionally substituted C₁-C₆ aliphatic.

25. The compound of any one of claims 1-24, wherein L² is optionally substituted C₁₋₃ alkylene.

26. The compound of any one of claims 1-24, wherein L² is optionally substituted C₁₋₃ alkylene, wherein one or more methylene units of the alkylene are independently replaced with —O—, —N(R⁴)—, —S—, —CH═N—, or —CH═CH—.

27. The compound of any one of claims 1-24, wherein L² is —C(R⁴)(R⁵)—N(R⁴)— or —C(R⁴)(R⁵)—O—.

28. The compound of claim 27, wherein R⁴ and R⁵ are taken together with the atom to which they are attached to form an optionally substituted 3-10 membered ring having 0-4 heteroatoms.

29. The compound of claim 27, wherein R⁵ is R.

30. The compound of any one of claims 1-29, wherein L² is optionally substituted C₁₋₃ alkylene, wherein one or more methylene units of the alkylene are independently replaced with optionally substituted -Cy-.

31. The compound of any one of claims 1-30, wherein L² is -Cy-O—.

32. The compound of any one of claims 1-31, wherein the compound have the structure of 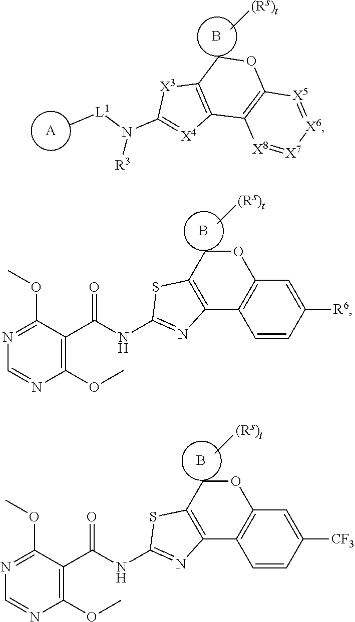
or a salt thereof.

33. The compound of any one claim 32, wherein Ring B in the -Cy- is an optionally substituted saturated 3-10 membered ring having 0-4 heteroatoms.

34. The compound of claim 1, wherein the compound has the structure of 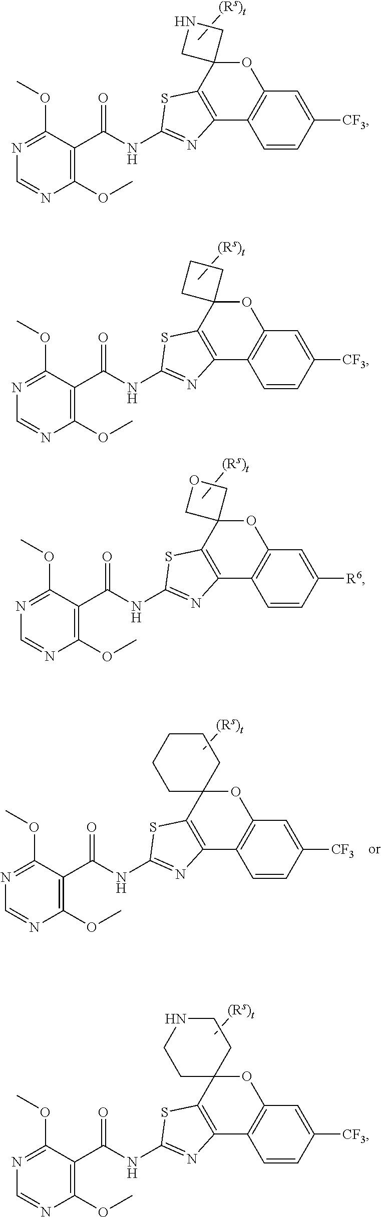
or a salt thereof.

35. A compound, wherein the compound is selected from 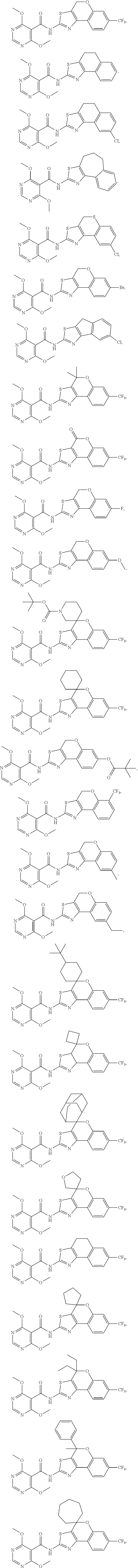 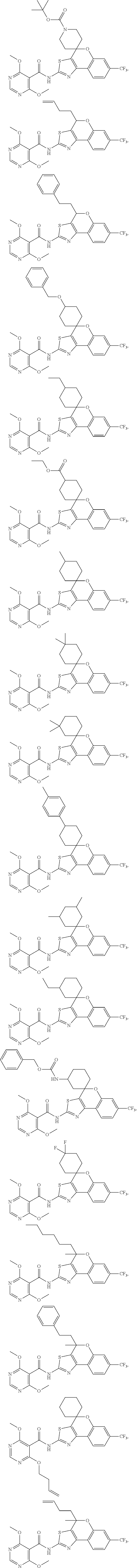 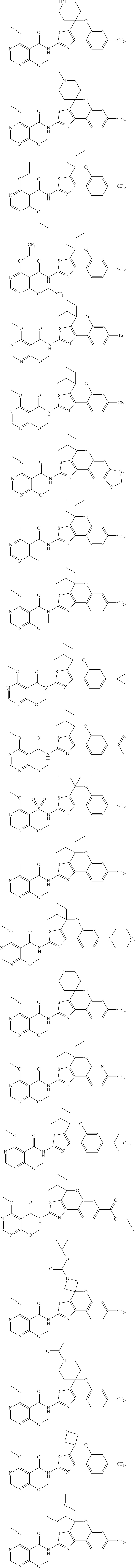 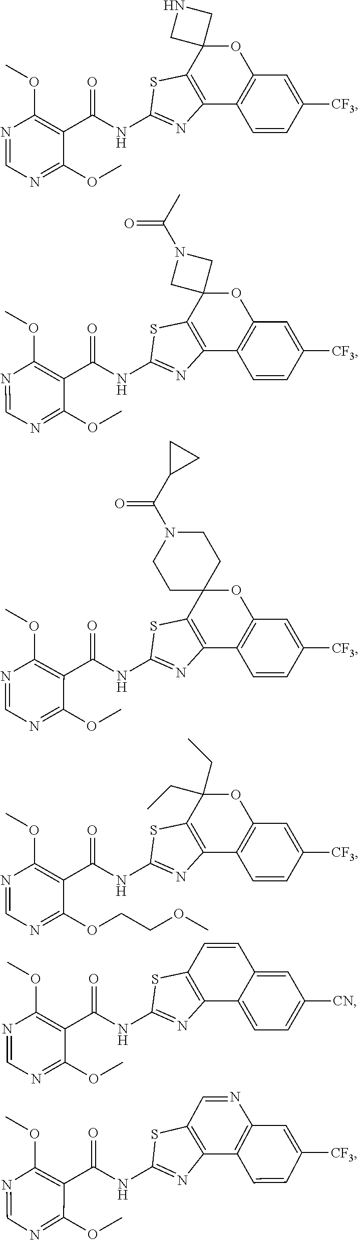 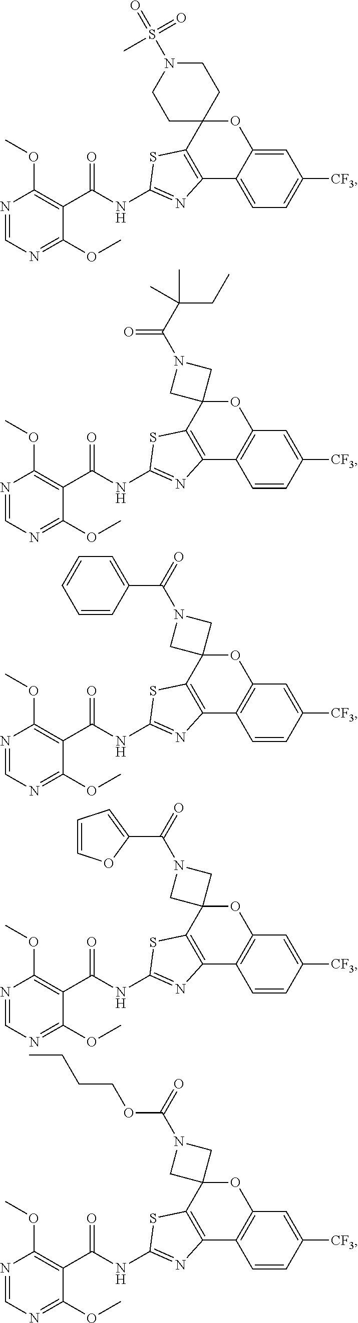 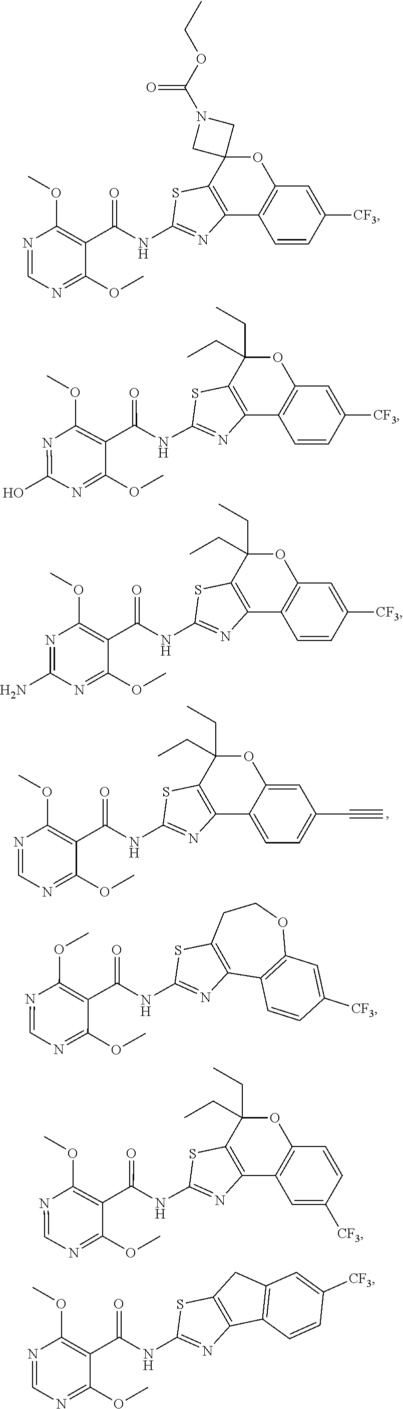 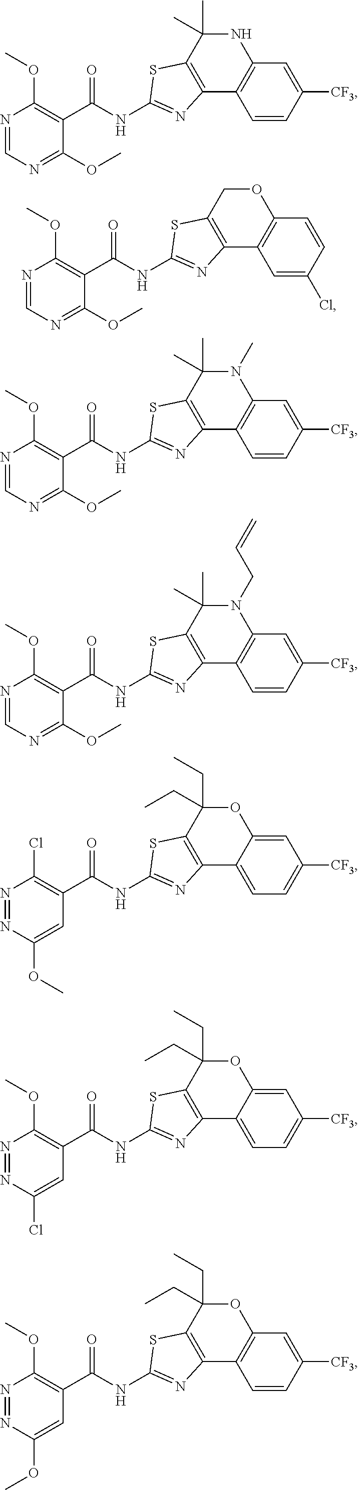 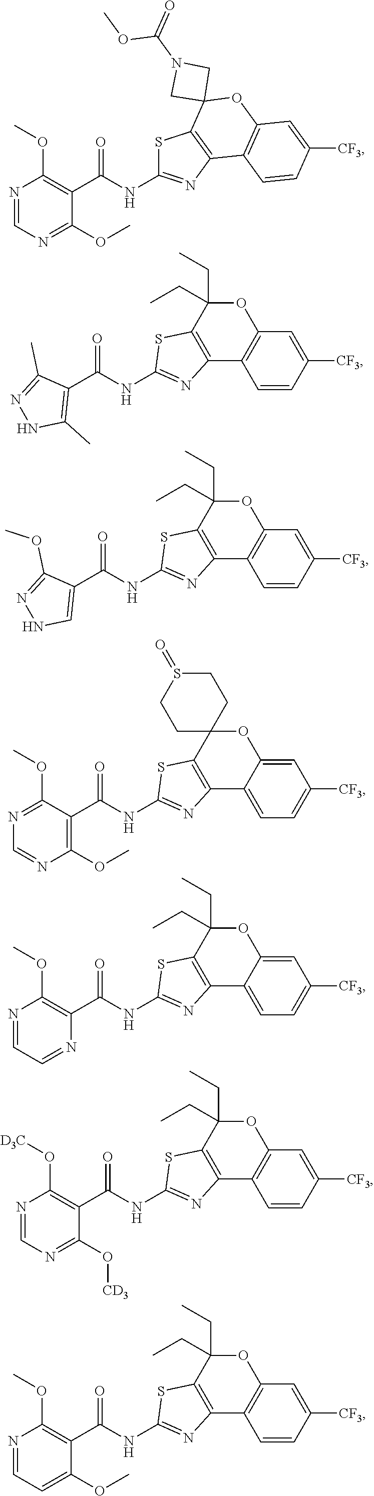 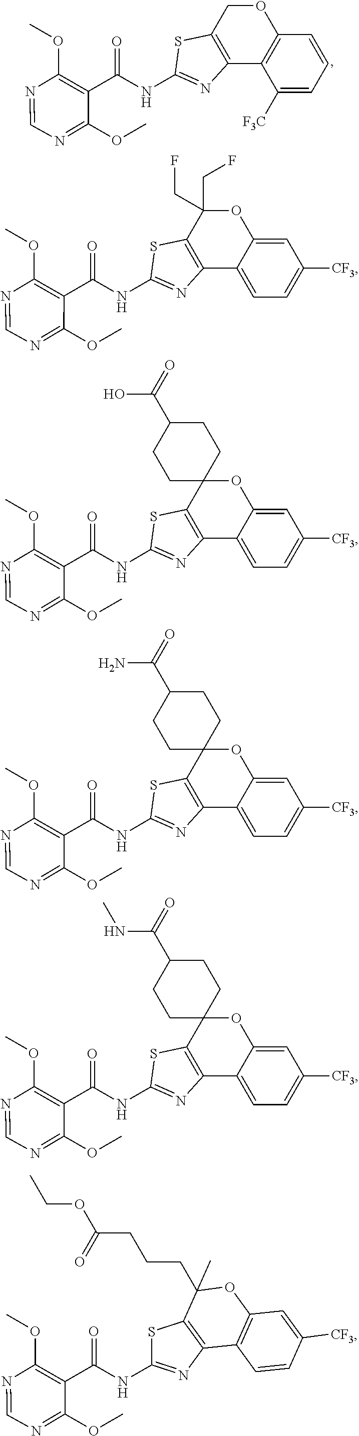 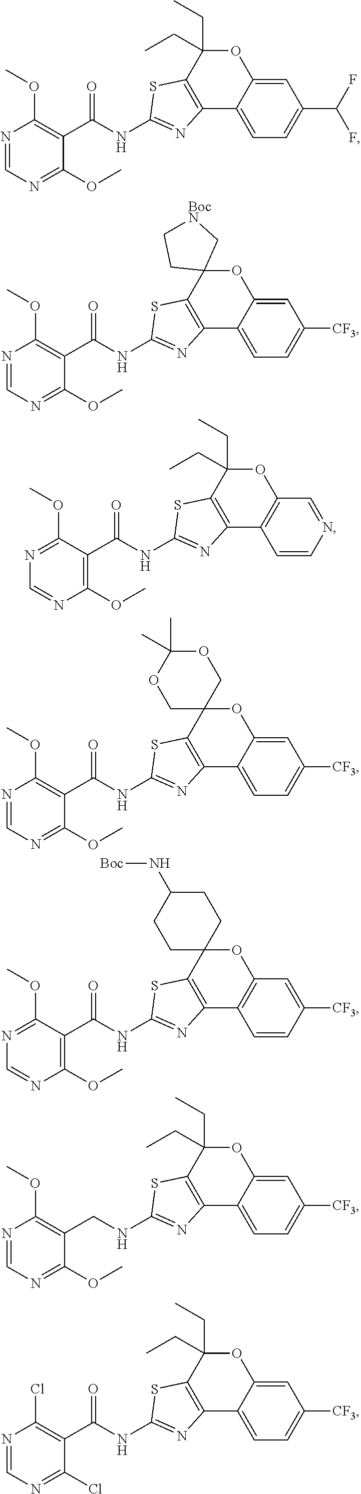 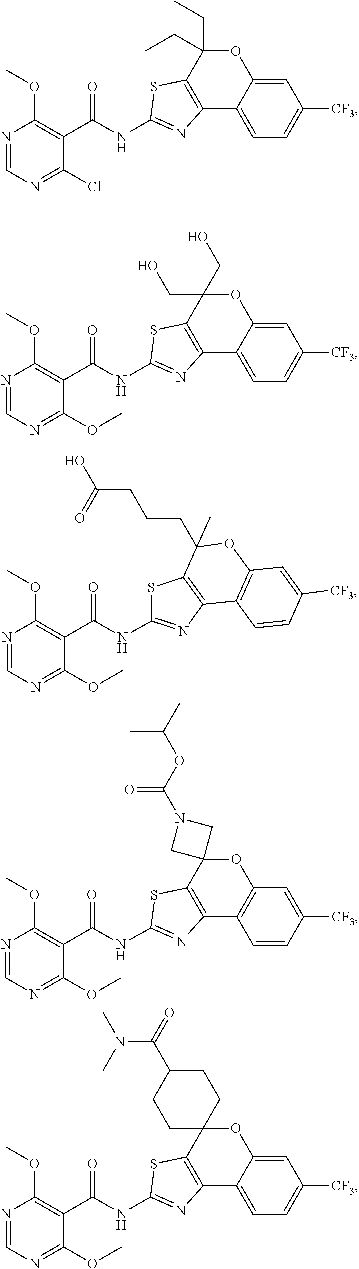 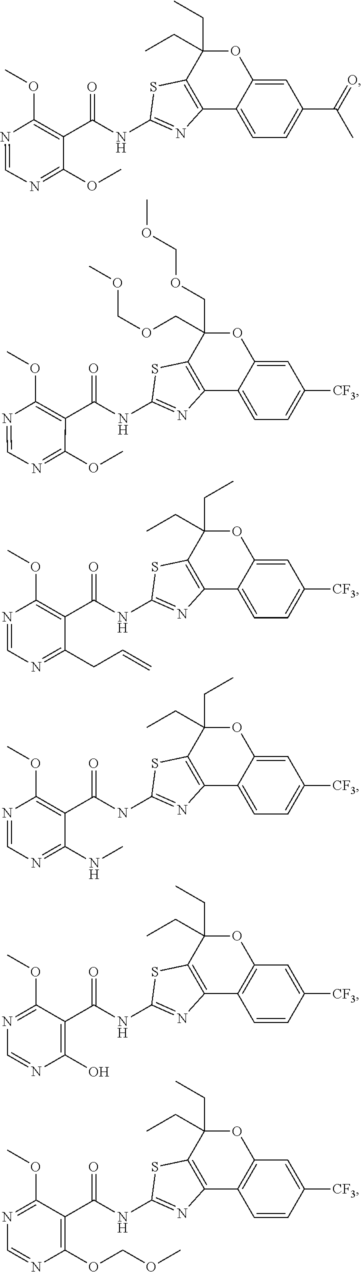 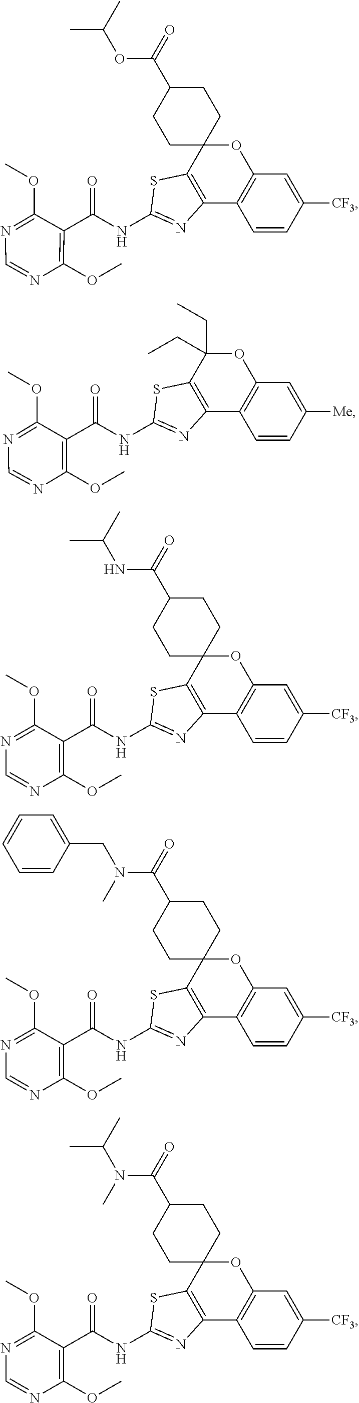 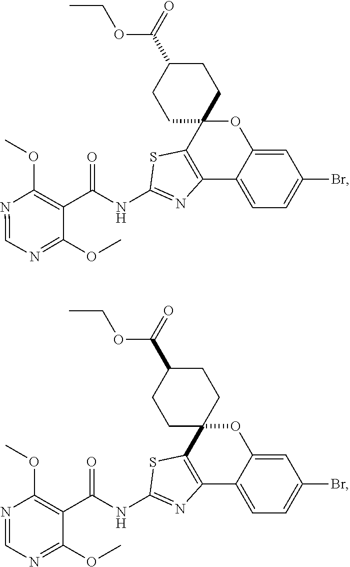 [SMILES: CCOC(=O)[C@H]1CC[C@]2(CC1)Oc1cc(Br)ccc1-c1nc(NC(=O)c3c(OC)ncnc3OC)sc12 ; CCOC(=O)[C@H]1CC[C@@]2(CC1)Oc1cc(Br)ccc1-c1nc(NC(=O)c3c(OC)ncnc3OC)sc12] 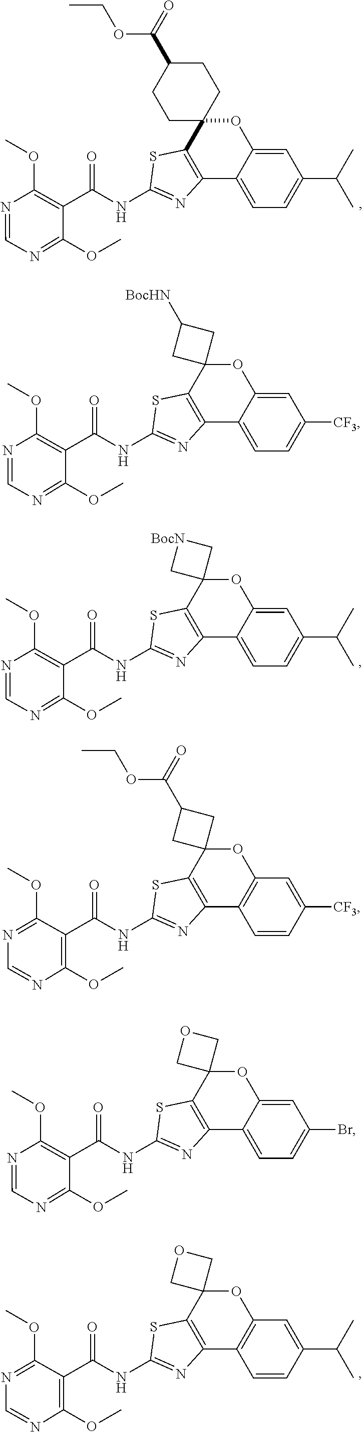 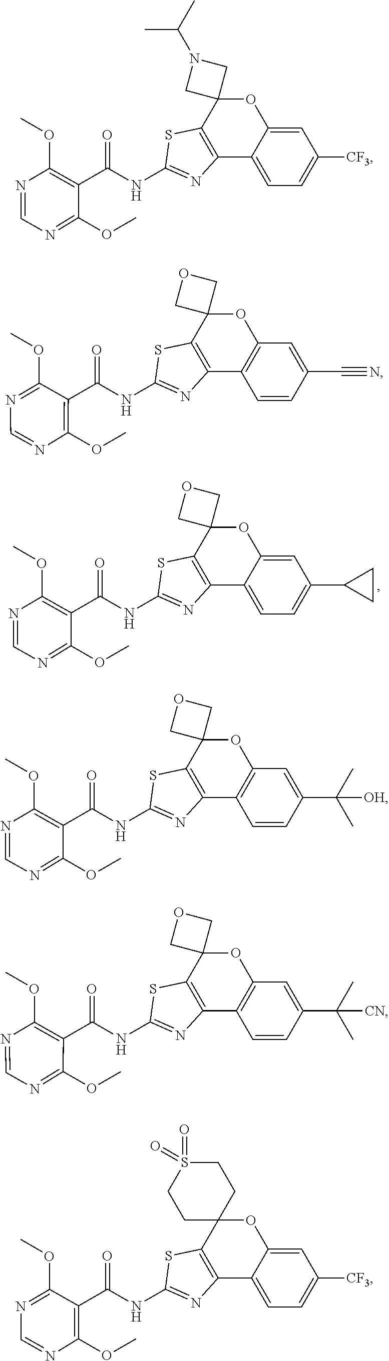 [SMILES: COc1ncnc(OC)c1C(=O)Nc1nc2c(s1)C1(CN(C(C)C)C1)Oc1cc(C(F)(F)F)ccc1-2 ; COc1ncnc(OC)c1C(=O)Nc1nc2c(s1)C1(COC1)Oc1cc(C#N)ccc1-2 ; COc1ncnc(OC)c1C(=O)Nc1nc2c(s1)C1(COC1)Oc1cc(C3CC3)ccc1-2 ; COc1ncnc(OC)c1C(=O)Nc1nc2c(s1)C1(COC1)Oc1cc(C(C)(C)O)ccc1-2 ; COc1ncnc(OC)c1C(=O)Nc1nc2c(s1)C1(COC1)Oc1cc(C(C)(C)C#N)ccc1-2 ; COc1ncnc(OC)c1C(=O)Nc1nc2c(s1)C1(CCS(=O)(=O)CC1)Oc1cc(C(F)(F)F)ccc1-2] 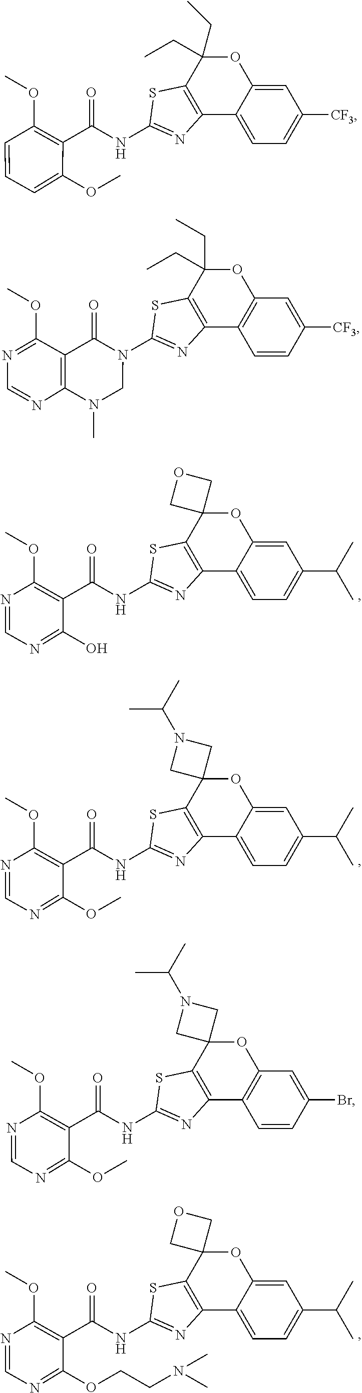 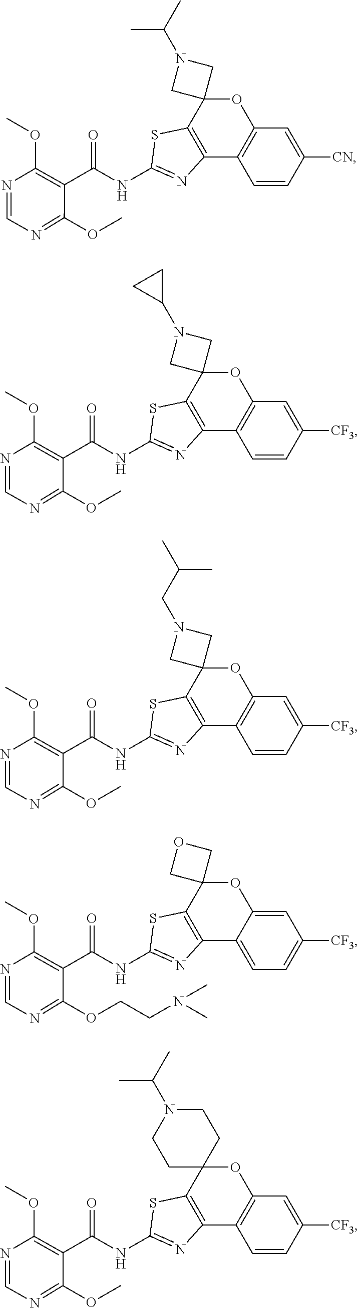 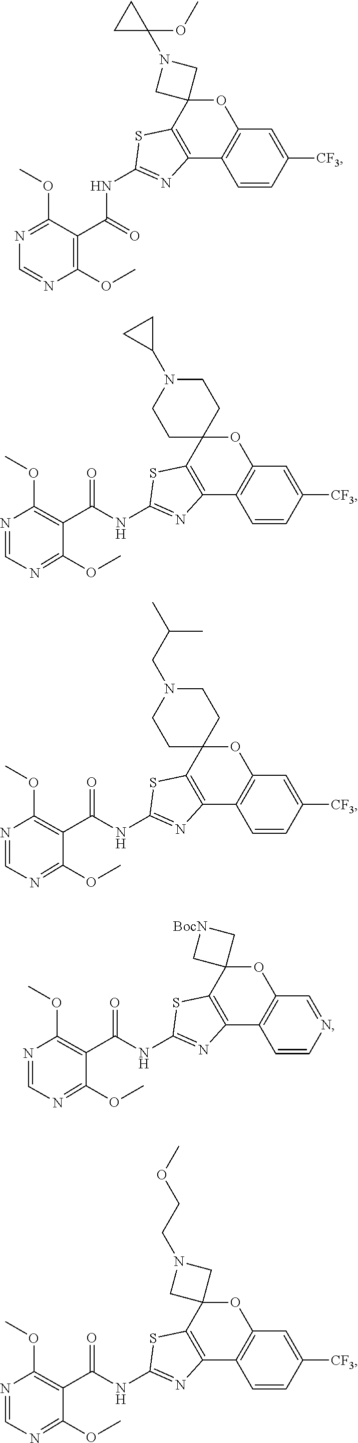 [SMILES: COc1ncnc(OC)c1C(=O)Nc1nc2c(s1)C1(CN(C3(OC)CC3)C1)Oc1cc(C(F)(F)F)ccc1-2 ; COc1ncnc(OC)c1C(=O)Nc1nc2c(s1)C1(CCN(C3CC3)CC1)Oc1cc(C(F)(F)F)ccc1-2 ; COc1ncnc(OC)c1C(=O)Nc1nc2c(s1)C1(CCN(CC(C)C)CC1)Oc1cc(C(F)(F)F)ccc1-2 ; COc1ncnc(OC)c1C(=O)Nc1nc2c(s1)C1(CN(C(=O)OC(C)(C)C)C1)Oc1cnccc1-2 ; COCCN1CC2(C1)Oc1cc(C(F)(F)F)ccc1-c1nc(NC(=O)c3c(OC)ncnc3OC)sc12] 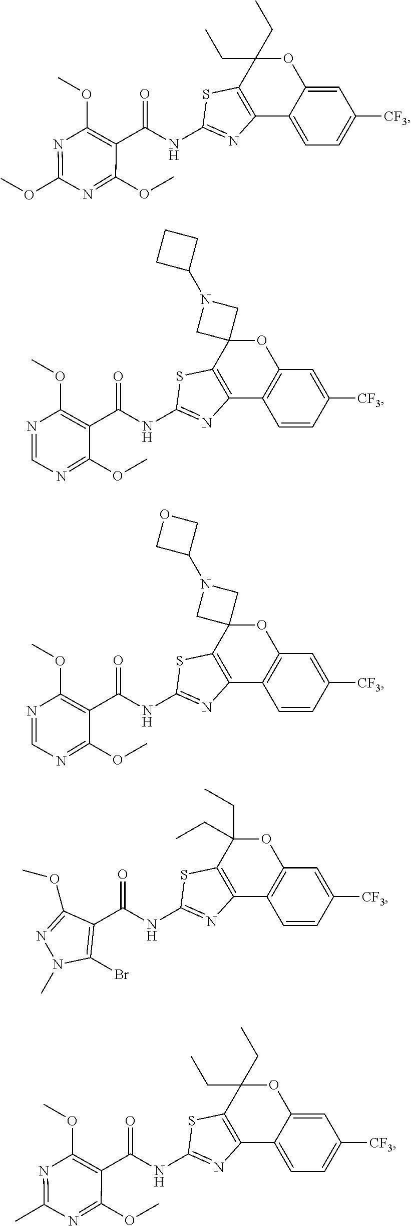 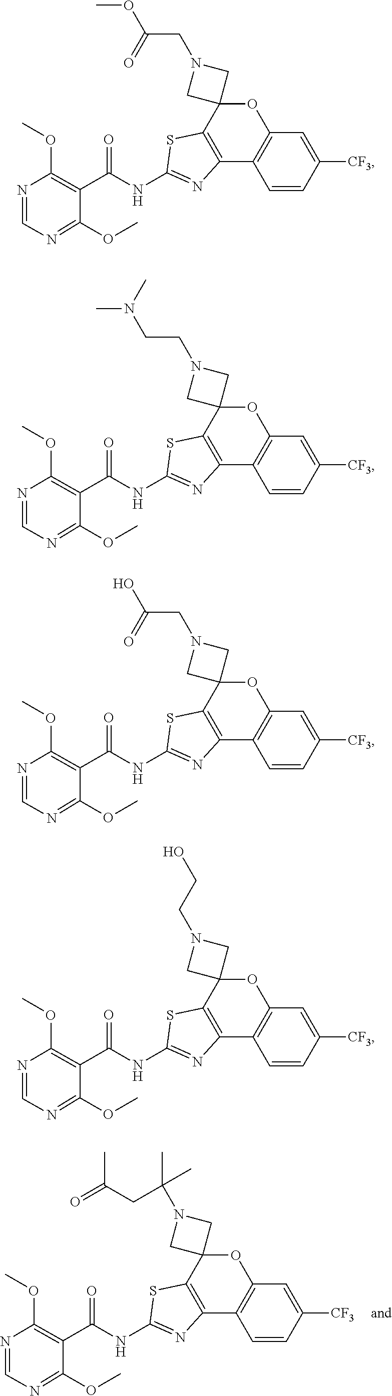 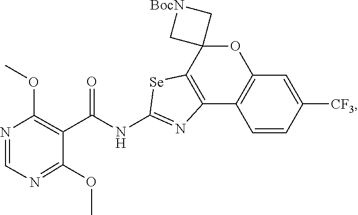 [SMILES: COc1ncnc(OC)c1C(=O)Nc1nc2c([se]1)C1(CN(C(=O)OC(C)(C)C)C1)Oc1cc(C(F)(F)F)ccc1-2]
or a salt thereof.

36. A pharmaceutical composition comprising a compound of any one of the preceding claims and a pharmaceutically acceptable carrier.

37. A method for treating a condition, disorder or disease, comprising administering to a subject suffering therefrom an effective amount of the compound or pharmaceutical composition of any one of claims 1-36.

38. The method of claim 37, wherein the condition, disorder or disease is or comprises migraine, arthralgia, cardiac pain arising from an ischemic myocardium, acute pain, chronic pain, nociceptive pain, neuropathic pain, post-operative pain, pain due to neuralgia (e.g., post-herpetic neuralgia, traumatic neuralgia, fibromyalgia, trigeminal neuralgia), pain due to diabetic neuropathy, dental pain, cancer pain, or inflammatory pain conditions (e.g. arthritis and osteoarthritis).

39. The method of claim 37, wherein the condition, disorder or disease is or comprises neuropathic pain, nociceptive pain, dental pain, HIV pain, cardiac pain arising from an ischemic myocardium, pain due to migraine, arthralgia, neuropathies, neurodegeneration, retinopathy, neurotic skin disorder, stroke, urinary bladder hypersensitiveness, urinary incontinence, vulvodynia, gastrointestinal disorders such as irritable bowel syndrome, gastro-esophageal reflux disease, enteritis, ileitis, stomach-duodenal ulcer, inflammatory bowel disease, Crohn's disease, celiac disease, an inflammatory disease such as pancreatitis, a respiratory disorder such as allergic and non-allergic rhinitis, asthma or chronic obstructive pulmonary disease, irritation of skin, eye or mucous membrane, atopic dermatitis, eczema itch, fervescence, muscle spasms, emesis, dyskinesias, depression, Huntington's disease, memory deficits, restricted brain function, amyotrophic lateral sclerosis (ALS), dementia, arthritis, osteoarthritis, diabetes, obesity, urticaria, actinic keratosis, keratocanthoma, alopecia, Meniere's disease, tinnitus, hyperacusis, anxiety disorders, or benign prostate hyperplasia.

40. The method of claim 37, wherein the condition, disorder or disease is or comprises atopic dermatitis, eczema, sebhorreic eczema, itch, skin inflammation, or psoriasis.

41. The method of claim 37, wherein the condition, disorder or disease is acne.

42. The method of claim 37, wherein the condition, disorder or disease is rosacea.

43. The method of any one of claims 37-42, wherein a compound or composition is administered or delivered orally.

44. The method of any one of claims 37-42, wherein a compound or composition is administered or delivered topically.
